(19) United States
(12) Patent Application Publication
(10) Pub. No.: US 2015/0301058 A1
(43) Pub. Date: Oct. 22, 2015

(54) BIOMARKER COMPOSITIONS AND METHODS

(71) Applicant: CARIS SCIENCE, INC., Irving, TX (US)

(72) Inventors: Jorge Schettini, Cambridge, MA (US); Tassilo Hornung, Tempe, AZ (US); Daniel Holterman, Phoenix, AZ (US); David Spetzler, Paradise Valley, AZ (US)

(21) Appl. No.: 14/646,708

(22) PCT Filed: Nov. 26, 2013
(86) PCT No.: PCT/US2013/072019

§ 371 Date: May 21, 2015

Related U.S. Application Data
(60) Provisional application No. 61/729,960, filed on Nov. 26, 2012.
(60) Provisional application No. 61/729,986, filed on Nov. 26, 2012.
(60) Provisional application No. 61/731,419, filed on Nov. 29, 2012.
(60) Provisional application No. 61/735,915, filed on Dec. 11, 2012.
(60) Provisional application No. 61/748,437, filed on Jan. 2, 2013.
(60) Provisional application No. 61/749,773, filed on Jan. 7, 2013.
(60) Provisional application No. 61/750,331, filed on Jan. 8, 2013.
(60) Provisional application No. 61/753,841, filed on Jan. 17, 2013.
(60) Provisional application No. 61/754,471, filed on Jan. 18, 2013.
(60) Provisional application No. 61/762,490, filed on Feb. 8, 2013.
(60) Provisional application No. 61/767,131, filed on Feb. 20, 2013.
(60) Provisional application No. 61/769,064, filed on Feb. 25, 2013.
(60) Provisional application No. 61/785,387, filed on Mar. 14, 2013.
(60) Provisional application No. 61/785,468, filed on Mar. 14, 2013.
(60) Provisional application No. 61/805,365, filed on Mar. 26, 2013.
(60) Provisional application No. 61/808,144, filed on Apr. 3, 2013.
(60) Provisional application No. 61/820,419, filed on May 7, 2013.
(60) Provisional application No. 61/826,957, filed on May 23, 2013.
(60) Provisional application No. 61/838,762, filed on Jun. 24, 2013.
(60) Provisional application No. 61/843,256, filed on Jul. 5, 2013.
(60) Provisional application No. 61/862,809, filed on Aug. 6, 2013.
(60) Provisional application No. 61/863,828, filed on Aug. 8, 2013.
(60) Provisional application No. 61/866,014, filed on Aug. 14, 2013.
(60) Provisional application No. 61/867,978, filed on Aug. 20, 2013.
(60) Provisional application No. 61/871,107, filed on Aug. 28, 2013.
(60) Provisional application No. 61/874,621, filed on Sep. 6, 2013.

(51) Int. Cl. G01N 33/574; A61K 39/00; C12Q 1/68; C12N 15/115

(52) U.S. Cl. CPC G01N 33/57488; C12N 15/115; A61K 39/0011; C12Q 1/6886; C12Q 2600/178; C12N 2310/16; C12N 2310/3513; A61K 2039/6025; C12Q 2600/158

(57) ABSTRACT

Biomarkers can be assessed for diagnostic, therapy-related or prognostic methods to identify phenotypes, such as a condition or disease, or the stage or progression of a disease, select candidate treatment regimens for diseases, conditions, disease stages, and stages of a condition, and to determine treatment efficacy. Circulating biomarkers from a bodily fluid can be used in profiling of physiological states or determining phenotypes. These include nucleic acids, protein, and circulating structures such as vesicles, and nucleic acid-protein complexes. The invention provides methods of assessing microvesicles in a biological sample. The invention also provides an aptamer to a microvesicle surface antigen. The aptamer may be used for therapeutic purposes.

### CROSS REFERENCE

This application claims the benefit of U.S. Provisional Patent Application Nos. 61/729,960, filed Nov. 26, 2012; 61/729,986, filed Nov. 26, 2012; 61/731,419, filed Nov. 29, 2012; 61/735,915, filed Dec. 11, 2012; 61/748,437, filed Jan. 2, 2013; 61/749,773, filed Jan. 7, 2013; 61/750,331, filed Jan. 8, 2013; 61/753,841, filed Jan. 17, 2013; 61/754,471, filed Jan. 18, 2013; 61/762,490, filed Feb. 8, 2013; 61/767,131, filed Feb. 20, 2013; 61/769,064, filed Feb. 25, 2013; 61/785,387, filed Mar. 14, 2013; 61/785,468, filed Mar. 14, 2013; 61/805,365, filed Mar. 26, 2013; 61/808,144, filed Apr. 3, 2013; 61/820,419, filed May 7, 2013; 61/826,957, filed May 23, 2013; 61/838,762, filed Jun. 24, 2013; 61/843,256, filed Jul. 5, 2013; 61/862,809, filed Aug. 6, 2013; 61/863,828, filed Aug. 8, 2013; 61/866,014, filed Aug. 14, 2013; 61/867,978, filed Aug. 20, 2013; 61/871,107, filed Aug. 28, 2013; and 61/874,621, filed Sep. 6, 2013; all of which applications are incorporated herein by reference in their entirety.

### SEQUENCE LISTING SUBMITTED VIA EFS-WEB

The entire content of the following electronic submission of the sequence listing via the USPTO EFS-WEB server, as authorized and set forth in MPEP §1730 II.B.2(a), is incorporated herein by reference in its entirety for all purposes. The sequence listing is within the electronically filed text file that is identified as follows:

File Name: 814601SequenceListing.txt

Date of Creation: Nov. 26, 2013

Size (bytes): 9,780 bytes

### BACKGROUND

Biomarkers for conditions and diseases such as cancer include biological molecules such as proteins, peptides, lipids, RNAs, DNA and variations and modifications thereof.

The identification of specific biomarkers, such as DNA, RNA and proteins, can provide biosignatures that are used for the diagnosis, prognosis, or theranosis of conditions or diseases. Biomarkers can be detected in bodily fluids, including circulating DNA, RNA, proteins, and vesicles. Circulating biomarkers include proteins such as PSA and CA125, and nucleic acids such as SEPT9 DNA and PCA3 messenger RNA (mRNA). Circulating biomarkers can be associated with circulating vesicles. Vesicles are membrane encapsulated structures that are shed from cells and have been found in a number of bodily fluids, including blood, plasma, serum, breast milk, ascites, bronchoalveolar lavage fluid and urine. Vesicles can take part in the communication between cells as transport vehicles for proteins, RNAs, DNAs, viruses, and prions. MicroRNAs are short RNAs that regulate the transcription and degradation of messenger RNAs. MicroRNAs have been found in bodily fluids and have been observed as a component within vesicles shed from tumor cells. The analysis of circulating biomarkers associated with diseases, including vesicles and/or microRNA, can aid in detection of disease or severity thereof, determining predisposition to a disease, as well as making treatment decisions.

Vesicles present in a biological sample provide a source of biomarkers, e.g., the markers are present within a vesicle (vesicle payload), or are present on the surface of a vesicle. Characteristics of vesicles (e.g., size, surface antigens, determination of cell-of-origin, payload) can also provide a diagnostic, prognostic or theranostic readout. There remains a need to identify biomarkers that can be used to detect and treat disease. microRNA, proteins and other biomarkers associated with vesicles as well as the characteristics of a vesicle can provide a diagnosis, prognosis, or theranosis.

The present invention provides methods and systems for characterizing a phenotype by detecting biomarkers that are indicative of disease or disease progress. The biomarkers can be circulating biomarkers including without limitation vesicle markers, protein, nucleic acids, mRNA, or and microRNA. The biomarkers can be nucleic acid-protein complexes. The methods of the invention comprise methods of detecting microvesicles in a sample. The invention also provides an aptamer capable of inhibiting microvesicle mediated immune suppression.

### SUMMARY

Disclosed herein are methods and compositions for characterizing a phenotype by analyzing circulating biomarkers, such as a vesicle, microRNA or protein present in a biological sample. Characterizing a phenotype for a subject or individual may include, but is not limited to, the diagnosis of a disease or condition, the prognosis of a disease or condition, the determination of a disease stage or a condition stage, a drug efficacy, a physiological condition, organ distress or organ rejection, disease or condition progression, therapy-related association to a disease or condition, or a specific physiological or biological state.

In an aspect, the invention provides a method for detecting a microvesicle population from a biological sample comprising: a) concentrating the biological sample using a selection membrane having a pore size of from 0.01 μm to about 10 μm, or a molecular weight cut off (MWCO) from about 1 kDa to 10000 kDa; b) diluting a retentate from the concentration step into one or more aliquots; and c) contacting each of the one or more aliquots of retentate with one or more binding agent specific to a molecule of at least one microvesicle in the microvesicle population. In a related aspect, the invention provides a method for detecting a microvesicle population from a biological sample comprising: a) concentrating the biological sample using a selection membrane having a pore size of from 0.01 μm to about 10 μm, or a molecular weight cut off (MWCO) from about 1 kDa to 10000 kDa; and b) contacting one or more aliquots of the retentate from the concentrating step with one or more binding agent specific to a molecule of at least one microvesicle in the microvesicle population. The selection membrane can be chosen to retain microvesicles while allowing smaller biological entities to pass into the filtrate. For example, the selection membrane can have a pore size of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, 3.0, 4.0, 5.0, 6.0, 7.0, 8.0, 9.0 or 10.0 μm. Alternately, the selection membrane can have a MWCO of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 2000, 3000, 4000, 5000, 6000, 7000, 8000, 9000 or 10000 kDa.

The retentate can be diluted into any number of desired aliquots. In various embodiments of the method, the retentate is diluted into at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350 or 400 aliquots. The retentate can also be diluted into various aliquots at one or more desired dilution factor. For example, the retentate can be diluted into one or more aliquots at a dilution factor of about 0, 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000 and/or 100000. In one embodiment, the retentate is diluted into one or more aliquots at a dilution factor of about 500.

The retentate can be diluted into one or more aliquots at various dilution factors, e.g., in order to determine a concentration curve. In a non-limiting example, the retenate is diluted into aliquots having a dilution factor of about 100, 250, 500, 1000, 10000 and 100000. The method can comprise detecting an amount of microvesicles in each aliquot of retentate that formed a complex with the one or more binding agent. A linear range of the amount of microvesicles in each aliquot can be determined by comparing the detected amount of vesicles against each dilution factor. Accordingly, a concentration of the microvesicles in the biological sample can be determined by extrapolating the amount of microvesicles determined in one or more aliquot within the linear range.

In another aspect, the invention provides a method of detecting a presence or level of one or more microvesicle in a biological sample, comprising: a) contacting a biological sample with a lipid staining dye, wherein the biological sample comprises or is suspected to comprise the one or more microvesicle; and b) detecting the lipid staining dye in contact with the one or more microvesicle, thereby detecting the presence or level of the one or more microvesicle.

The lipid staining dye may comprise a long-chain dialkylcarbocyanine, an indocarbocyanine (DiI), an oxacarbocyanine (DiO), FM 1-43, FM 1-43FX, FM 4-64, FM 5-95, a dialkyl aminostyryl dye, DiA, a long-wavelength light-excitable carbocyanines (DiD), an infrared light-excitable carbocyanine (DiR), carboxyfluorescein succinimidyl ester (CFDA), carboxyfluorescein succinimidyl ester (CFSE), 4-(4-(Dihexadecylamino)styryl)-N-Methylpyridinium Iodide (DiA; 4-Di-16-ASP), 4-(4-(Didecylamino)styryl)-N-Methylpyridinium Iodide (4-Di-10-ASP), 1,1′-Dioctadecyl-3,3,3′,3′-Tetramethylindodicarbocyanine Perchlorate (‘DiD’ oil; DiIC18(5) oil), 1,1′-Dioctadecyl-3,3,3′,3′-Tetramethylindodicarbocyanine, 4-Chlorobenzenesulfonate Salt (‘DiD’ solid; DiIC18(5) solid), 1,1′-Dioleyl-3,3,3′,3′-Tetramethylindocarbocyanine methanesulfonate (49-DiI), Dil Stain (1,1′-Dioctadecyl-3,3,3′,3′-Tetramethylindocarbocyanine Perchlorate (‘DiI’; DiIC18(3))), Dil Stain (1,1′-Dioctadecyl-3,3,3′,3′-Tetramethylindocarbocyanine Perchlorate (‘DiI’; DiIC18(3))), 1,1′-Didodecyl-3,3,3′,3′-Tetramethylindocarbocyanine Perchlorate (DiIC12(3)), 1,1′-Dihexadecyl-3,3,3′,3′-Tetramethylindocarbocyanine Perchlorate (DiIC16(3)), 1,1′-Dioctadecyl-3,3,3′,3′-Tetramethylindocarbocyanine-5,5′-Disulfonic Acid (DiIC18(3)-DS), 1,1′-Dioctadecyl-3,3,3′,3′-Tetramethylindodicarbocyanine-5,5′-Disulfonic Acid (DiIC18(5)-DS), 4-(4-(Dilinoleylamino)styryl)-N-Methylpyridinium 4-Chlorobenzenesulfonate (FAST DiA™ solid; DiΔ9,12-C18ASP, CBS), 3,3′-Dilinoleyloxacarbocyanine Perchlorate (FAST DiO™ Solid; DiOΔ9,12-C18(3), ClO4), 1,1′-Dilinoleyl-3,3,3′,3′-Tetramethylindocarbocyanine, 4-Chlorobenzenesulfonate (FAST DiI™ solid; DiIΔ9,12-C18(3), CBS), 1,1′-Dilinoleyl-3,3,3′,3′-Tetramethylindocarbocyanine Perchlorate (FAST DiI™ oil; DiIΔ9,12-C18(3), ClO4), 3,3′-Dioctadecyloxacarbocyanine Perchlorate (‘DiO’; DiOC18(3)), 3,3′-Dihexadecyloxacarbocyanine Perchlorate (DiOC16(3)), 3,3′-Dioctadecyl-5,5′-Di(4-Sulfophenyl)Oxacarbocyanine, Sodium Salt (SP-DiOC18(3)), 1,1′-Dioctadecyl-6,6′-Di(4-Sulfophenyl)-3,3,3′,3′-Tetramethylindocarbocyanine (SP-DiIC18(3)), 1,1′-Dioctadecyl-3,3,3′,3′-Tetramethylindotricarbocyanine Iodide (DiR; DiIC18(7)), 3,3′-Diethylthiacarbocyanine iodide, 3,3′-Diheptylthiacarbocyanine iodide, 3,3′-Dioctylthiacarbocyanine iodide, 3,3′-Dipropylthiadicarbocyanine iodide, 7-(Diethylamino)coumarin-3-carboxylic acid, 7-(Diethylamino)coumarin-3-carboxylic acid N-succinimidyl ester, an analog or variant of any thereof, and a combination of any thereof.

The lipid staining dye can be labeled. In some embodiments, the lipid staining dye is converted from a non-labeled form to a labeled form upon contact with the microvesicle. For example, the lipid staining dye can be an esterase-activated lipophilic dye, including without limitation the non-fluorescent carboxyfluorescein succinimidyl ester (CFDA). The CFDA can be converted into fluorescent carboxyfluorescein succinimidyl ester (CFSE) by vesicle esterases.

Steps (a)-(b) can be repeated to detect a level of one or more microvesicle in a series of biological samples having known microvesicle concentrations. A standard curve can be constructed from the detected levels. Steps (a)-(b) can then be performed to detect a level of one or more microvesicle in a test sample. The level in the test sample can be interpolated to the standard curve, thereby determining the microvesicle concentration in the test sample.

In yet another aspect, the invention provides a method of detecting a presence or level of one or more microvesicle in a biological sample, comprising: a) providing a biological sample comprising or suspected to comprise the one or more microvesicle; b) selectively depleting one or more abundant protein from the biological sample provided in step (a); and c) performing affinity selection of the one or more microvesicle from the sample depleted in step (b), thereby detecting the presence or level of one or more microvesicle. Selective depletion of abundant proteins can be performed in conjunction with other aspects of the invention, e.g., when filtering and/or diluting a sample, and/or in conjuction with a lipid staining dye.

In any of the various aspects of the invention, the biological sample may comprise a bodily fluid. The bodily fluid can include without limitation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, or a derivative of any thereof. For example, the biological sample may comprise peripheral blood, serum or plasma.

Abundant protein can be removed at various steps. For example, in some embodiments, the methods of the invention comprise selectively depleting one or more abundant protein from the biological sample prior to step (a). In other embodiments, the methods of the invention further comprise selectively depleting one or more abundant protein from the biological sample prior to step (b). Removal techniques can be performed at more than one step.

As noted, the biological sample may comprise peripheral blood, serum or plasma. The one or more abundant protein in blood can comprise one or more of albumin, IgG, transferrin, fibrinogen, fibrin, IgA, α2-Macroglobulin, IgM, α1-Antitrypsin, complement C3, haptoglobulin, apolipoprotein A1, A3 and B; α1-Acid Glycoprotein, ceruloplasmin, complement C4, C1q, IgD, prealbumin (transthyretin), plasminogen, a derivative of any thereof, and a combination thereof. The one or more abundant protein in blood can also be selected from the group consisting of Albumin, Immunoglobulins, Fibrinogen, Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin, Mannose-binding lectin (MBL), a derivative of any thereof, and a combination thereof.

Various techniques can be used to selectively deplete the one or more abundant protein. For example, selectively depleting the one or more abundant protein may comprise contacting the biological sample with thromboplastin to precipitate fibrinogen. In another example, the one or more abundant protein is depleted by immunoaffinity, precipitation, or a combination thereof.

Selectively depleting the one or more abundant protein from the biological sample may comprise partial or complete removal. For example, the methods of the invention may comprise depleting at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the one or more abundant protein.

In any of the various aspects of the invention, the biological sample may comprise a cell culture sample. Alternately, the biological sample may comprise a tissue sample. In some embodiments, the tissue sample comprises tumor cells.

In any of the various aspects of the invention, the method comprises detecting one or more microvesicle antigen associated with the one or more microvesicle. The microvesicle antigen can be selected to identify microvesicles shed from cells of various origins, e.g., from a diseased cell such as a cancer cell, or from a cell of a particular anatomical origin, e.g., from a particular organ of interest. In embodiments of the invention, the one or more microvesicle-associated antigen is selected from Table 3, Table 4, and/or Table 5. The one or more microvesicle-associated antigen can include a protein selected from the group consisting of ADAM 34, ADAM 9, AGR2, ALDOA, ANXA1, ANXA 11, ANXA4, ANXA 7, ANXA2, ARF6, ATP1A1, ATP1A2, ATP1A3, BCHE, BCL2L14 (Bcl G), BDKRB2, CA215, CAV1-Caveolin1, CCR2 (CC chemokine receptor 2, CD192), CCR5 (CC chemokine receptor 5), CCT2 (TCP1-beta), CD166/ALCAM, CD49b (Integrin alpha 2, ITGA4), CD90/THY1, CDH1, CDH2, CDKN1A cyclin-dependent kinase inhibitor (p21), CGA gene (coding for the alpha subunit of glycoprotein hormones), CHMP4B, CLDN3-Claudin3, CLSTN1 (Calsyntenin-1), COX2 (PTGS2), CSE1L (Cellular Apoptosis Susceptibility), Cytokeratin 18, Eag1 (KCNH1) (plasma membrane-K+-voltage gated channel), EDIL3 (del-1), EDNRB—Endothelial Receptor Type B, Endoglin/CD105, ENOX2-Ecto-NOX disulphide Thiol exchanger 2, EPCA-2 Early prostate cancer antigen2, EpoR, EZH2 (enhancer of Zeste Homolog2), EZR, FABP5, Farnesyltransferase/geranylgeranyl diphosphate synthase 1 (GGPS1), Fatty acid synthase (FASN, plasma membrane protein), FTL (light and heavy), GDF15-Growth Differentiation Factor 15, GloI, GSTP1, H3F3A, HGF (hepatocyte growth factor), hK2 (KLK2), HSP90AA1, HSPA1A/HSP70-1, IGFBP-2, IGFBP-3, IL1alpha, IL-6, IQGAP1, ITGAL (Integrin alpha L chain), Ki67, KLK1, KLK10, KLK11, KLK12, KLK13, KLK14, KLK15, KLK4, KLK5, KLK6, KLK7, KLK8, KLK9, Lamp-2, LDH-A, LGALS3BP, LGALS8, MFAP5, MMP 1, MMP 2, MMP 24, MMP 25, MMP 3, MMP10, MMP-14/MT1-MMP, MTA1, nAnS, Nav1.7, NCAM2—Neural cell Adhesion molecule 2, NGEP/D-TMPP/IPCA-5/ANO7, NKX3-1, Notch1, NRP1/CD304, PGP, PAP (ACPP), PCA3-Prostate cancer antigen 3, Pdia3/ERp57, PhIP, phosphatidylethanolamine (PE), PIP3, PKP1 (plakophilin1), PKP3 (plakophilin3), Plasma chromogranin-A (CgA), PRDX2, Prostate secretory protein (PSP94)/β-Microseminoprotein (MSP)/IGBF, PSAP, PSMA1, PTEN, PTGFRN, PTPN13/PTPL1, PKM2, RPL19, SCA-1/ATXN1, SERINC5/TPO1, SET, SLC3A2/CD98, STEAP1, STEAP-3, SRVN, Syndecan/CD138, TGFB, Tissue Polypeptide Specific antigen TPS, TLR4 (CD284), TLR9 (CD289), TMPRSS1/hepsin, TMPRSS2, TNFR1, TNFα, CD283/TLR3, Transferrin receptor/CD71/TRFR, uPA (urokinase plasminoge activator), uPAR (uPA receptor)/CD87, VEGFR1, VEGFR2, and a combination thereof. The one or more microvesicle-associated antigen can also include a protein selected from the group consisting of ADAM 9, ADAM10, AGR2, ALDOA, ALIX, ANXA1, ANXA2, ANXA4, ARF6, ATP1A3, B7H3, BCHE, BCL2L14 (Bcl G), BCNP1, BDKRB2, BDNFCAV1-Caveolinl, CCR2 (CC chemokine receptor 2, CD192), CCR5 (CC chemokine receptor 5), CCT2 (TCP1-beta), CD10, CD151, CD166/ALCAM, CD24, CD283/TLR3, CD41, CD46, CD49d (Integrin alpha 4, ITGA4), CD63, CD81, CD9, CD90/THY1, CDH1, CDH2, CDKN1A cyclin-dependent kinase inhibitor (p21), CGA gene (coding for the alpha subunit of glycoprotein hormones), CLDN3-Claudin3, COX2 (PTGS2), CSE1L (Cellular Apoptosis Susceptibility), CXCR3, Cytokeratin 18, Eag1 (KCNH1), EDIL3 (del-1), EDNRB-Endothelial Receptor Type B, EGFR, EpoR, EZH2 (enhancer of Zeste Homolog2), EZR, FABP5, Farnesyltransferase/geranylgeranyl diphosphate synthase 1 (GGPS1), Fatty acid synthase (FASN), FTL (light and heavy), GAL3, GDF15-Growth Differentiation Factor 15, GloI, GM-CSF, GSTP1, H3F3A, HGF (hepatocyte growth factor), hK2/Kif2a, HSP90AA1, HSPA1A/HSP70-1, HSPB1, IGFBP-2, IGFBP-3, IL1alpha, IL-6, IQGAP1, ITGAL (Integrin alpha L chain), Ki67, KLK1, KLK10, KLK11, KLK12, KLK13, KLK14, KLK15, KLK4, KLK5, KLK6, KLK7, KLK8, KLK9, Lamp-2, LDH-A, LGALS3BP, LGALS8, MMP 1, MMP 2, MMP 25, MMP 3, MMP10, MMP-14/MT1-MMP, MMP7, MTA1nAnS, Nav1.7, NKX3-1, Notch1, NRP1/CD304, PAP (ACPP), PGP, PhIP, PIP3/BPNT1, PKM2, PKP1 (plakophilin1), PKP3 (plakophilin3), Plasma chromogranin-A (CgA), PRDX2, Prostate secretory protein (PSP94)/β-Microseminoprotein (MSP)/IGBF, PSAP, PSMA, PSMA1, PTENPTPN13/PTPL1, RPL19, seprase/FAPSET, SLC3A2/CD98, SRVN, STEAP1, Syndecan/CD138, TGFB, TGM2, TIMP-1TLR4 (CD284), TLR9 (CD289), TMPRSS1/hepsin, TMPRSS2, TNFR1, TNFα, Transferrin receptor/CD71/TRFR, Trop2 (TACSTD2), TWEAK uPA (urokinase plasminoge activator) degrades extracellular matrix, uPAR (uPA receptor)/CD87, VEGFR1, VEGFR2, and a combination thereof. In some embodiments, the one or more microvesicle-associated antigen comprises a protein selected from the group consisting of A33, ABL2, ADAM10, AFP, ALA, ALIX, ALPL, ApoJ/CLU, ASCA, ASPH(A-10), ASPH(D01P), AURKB, B7H3, B7H3, B7H4, BCNP, BDNF, CA125(MUC16), CA-19-9, C-Bir, CD10, CD151, CD24, CD41, CD44, CD46, CD59(MEM-43), CD63, CD63, CD66eCEA, CD81, CD81, CD9, CD9, CDA, CDADC1, CRMP-2, CRP, CXCL12, CXCR3, CYFRA21-1, DDX-1, DLL4, DLL4, EGFR, Epcam, EphA2, ErbB2, ERG, EZH2, FASL, FLNA, FRT, GAL3, GATA2, GM-CSF, Gro-alpha, HAP, HER3(ErbB3), HSP70, HSPB1, hVEGFR2, iC3b, IL-1B, IL6R, IL6Unc, IL7Ralpha/CD127, IL8, INSIG-2, Integrin, KLK2, LAMN, Mammoglobin, M-CSF, MFG-E8, MIF, MISRII, MMP7, MMP9, MUC1, Muc1, MUC17, MUC2, Ncam, NDUFB7, NGAL, NK-2R(C-21), NT5E (CD73), p53, PBP, PCSA, PCSA, PDGFRB, PIM1, PRL, PSA, PSA, PSMA, PSMA, RAGE, RANK, RegIV, RUNX2, S100-A4, seprase/FAP, SERPINB3, SIM2(C-15), SPARC, SPC, SPDEF, SPP1, STEAP, STEAP4, TFF3, TGM2, TIMP-1, TMEM211, Trail-R2, Trail-R4, TrKB(poly), Trop2, Tsg101, TWEAK, UNC93A, VEGFA, wnt-5a(C-16), and a combination thereof.

The microvesicles can be detected using a combination of binding agent against various antigens. For example, the one or more microvesicle-associated antigen can comprise one or more of the biomarkers listed above and further comprise a protein selected from the group consisting of CD9, CD63, CD81, PCSA, MUC2, MFG-E8, and a combination thereof.

In still other embodiments, the one or more biomarker comprises a protein selected from the group consisting of A33, ADAM10, AMACR, ASPH (A-10), AURKB, B7H3, CA125, CA-19-9, C-Bir, CD24, CD3, CD41, CD63, CD66e CEA, CD81, CD9, CDADC1, CSA, CXCL12, DCRN, EGFR, EphA2, ERG, FLNA, FRT, GAL3, GM-CSF, Gro-alpha, HER 3 (ErbB3), hVEGFR2, IL6 Unc, Integrin, Mammaglobin, MFG-E8, MMP9, MUC1, MUC17, MUC2, NGAL, NK-2R(C-21), NY-ESO-1, PBP, PCSA, PIM1, PRL, PSA, PSIP1/LEDGF, PSMA, RANK, S100-A4, seprase/FAP, SIM2 (C-15), SPDEF, SSX2, STEAP, TGM2, TIMP-1, Trail-R4, Tsg 101, TWEAK, UNC93A, VCAN, XAGE-1, and a combination thereof. The one or more biomarker may further comprise a protein selected from the group consisting of EpCAM, CD81, PCSA, MUC2, MFG-E8, and a combination thereof. In some embodiments, the biosignature is used to characterize a prostate cancer.

In still other embodiments, the one or more biomarker comprises a protein selected from the group consisting of the one or more biomarker comprises a protein selected from the group consisting of A33, ADAM10, ALIX, AMACR, ASCA, ASPH (A-10), AURKB, B7H3, BCNP, CA125, CA-19-9, C-Bir (Flagellin), CD24, CD3, CD41, CD63, CD66e CEA, CD81, CD9, CDADC1, CRP, CSA, CXCL12, CYFRA21-1, DCRN, EGFR, EpCAM, EphA2, ERG, FLNA, GAL3, GATA2, GM-CSF, Gro alpha, HER3 (ErbB3), HSP70, hVEGFR2, iC3b, IL-1B, IL6 Unc, IL8, Integrin, KLK2, Mammaglobin, MFG-E8, MMP7, MMP9, MS4A1, MUC1, MUC17, MUC2, NGAL, NK-2R(C-21), NY-ESO-1, p53, PBP, PCSA, PIM1, PRL, PSA, PSMA, RANK, RUNX2, S100-A4, seprase/FAP, SERPINB3, SIM2 (C-15), SPC, SPDEF, SSX2, SSX4, STEAP, TGM2, TIMP-1, TRAIL R2, Trail-R4, Tsg 101, TWEAK, VCAN, VEGF A, XAGE, and a combination thereof. The one or more biomarker may further comprise a protein selected from the group consisting of EpCAM, CD81, PCSA, MUC2, MFG-E8, and a combination thereof. In some embodiments, the biosignature is used to characterize a cancer, e.g., a prostate cancer.

In an embodiment, the one or more biomarker comprises one or more protein selected from the group consisting of CD9, CD63, CD81, MMP7, EpCAM, and a combination thereof. The one or more biomarker can be a protein selected from the group consisting of STAT3, EZH2, p53, MACC1, SPDEF, RUNX2, YB-1, AURKA, AURKB, and a combination thereof. The one or more biomarker can be a protein selected from the group consisting of PCSA, Muc2, Adam10, and a combination thereof. The one or more biomarker can include MMP7. The biosignature can be used to detect a cancer, e.g., a breast or prostate cancer.

In another embodiment, the one or more biomarker comprises a protein selected from the group consisting of Alkaline Phosphatase (AP), CD63, MyoD1, Neuron Specific Enolase, MAP1B, CNPase, Prohibitin, CD45RO, Heat Shock Protein 27, Collagen II, Laminin B1/b1, Gail, CDw75, bcl-XL, Laminin-s, Ferritin, CD21, ADP-ribosylation Factor (ARF-6), and a combination thereof. The one or more biomarker may comprise a protein selected from the group consisting of CD56/NCAM-1, Heat Shock Protein 27/hsp27, CD45RO, MAP1B, MyoD1, CD45/T200/LCA, CD3zeta, Laminin-s, bcl-XL, Rad18, Gail, Thymidylate Synthase, Alkaline Phosphatase (AP), CD63, MMP-16/MT3-MMP, Cyclin C, Neuron Specific Enolase, SIRP a1, Laminin B1/b1, Amyloid Beta (APP), SODD (Silencer of Death Domain), CDC37, Gab-1, E2F-2, CD6, Mast Cell Chymase, Gamma Glutamylcysteine Synthetase (GCS), and a combination thereof. For example, the one or more biomarker may comprise a protein selected from the group consisting of Alkaline Phosphatase (AP), CD56 (NCAM), CD-3 zeta, Map1b, 14.3.3 pan, filamin, thrombospondin, and a combination thereof. The biosignature can be used to characterize a cancer. For example, the biosignature may be used to distinguish between a prostate cancer and other prostate disorders. The biosignature may also be used to distinguish between a prostate cancer and other cancers, e.g., lung, colorectal, breast and brain cancer.

In another embodiment, the one or more biomarker comprises a protein selected from the group consisting of ADAM-10, BCNP, CD9, EGFR, EpCam, IL1B, KLK2, MMP7, p53, PBP, PCSA, SERPINB3, SPDEF, SSX2, SSX4, and a combination thereof. For example, the one or more biomarker may comprise a protein selected from the group consisting of EGFR, EpCAM, KLK2, PBP, SPDEF, SSX2, SSX4, and a combination thereof. The one or more biomarker may also comprise a protein selected from the group consisting of EpCAM, KLK2, PBP, SPDEF, SSX2, SSX4, and a combination thereof.

The one or more microvesicle-associated antigen may comprise a pair of proteins selected from the pairs in any of Tables 28-40 and 44-46. For example, one microvesicle-associated antigen may be used for capture while another is used for detection. The one or more microvesicle-associated antigen can comprise a pair of proteins selected from any pairs of Mammaglobin-MFG-E8, SIM2-MFG-E8 and NK-2R-MFG-E8. The one or more microvesicle-associated antigen can comprise a pair of proteins selected from any pairs of Integrin-MFG-E8, NK-2R-MFG-E8 and Gal3-MFG-E8. The one or more microvesicle-associated antigen can comprise a pair of proteins selected from any pairs of one of AURKB, A33, CD63, Gro-alpha, and Integrin; and one of MUC2, PCSA, and CD81. The one or more microvesicle-associated antigen can comprise a pair of proteins selected from any pairs of one of AURKB, CD63, FLNA, A33, Gro-alpha, Integrin, CD24, SSX2, and SIM2; and one of MUC2, PCSA, CD81, MFG-E8, and EpCam. The one or more microvesicle-associated antigen can comprise a pair of proteins selected from any pairs of EpCam-MMP7, PCSA-MMP7, and EpCam-BCNP. The one or more microvesicle-associated antigen can comprise a pair of proteins selected from any pairs of EpCam-MMP7, PCSA-MMP7, EpCam-BCNP, PCSA-ADAM10, and PCSA-KLK2. The one or more microvesicle-associated antigen can comprise a pair of proteins selected from any pairs of EpCam-MMP7, PCSA-MMP7, EpCam-BCNP, PCSA-ADAM10, PCSA-KLK2, PCSA-SPDEF, CD81-MMP7, PCSA-EpCam, MFGE8-MMP7 and PCSA-IL-8. The one or more microvesicle-associated antigen can comprise a pair of proteins selected from any pairs of EpCam-MMP7, PCSA-MMP7, EpCam-BCNP, PCSA-ADAM10, and CD81-MMP7. The one or more microvesicle-associated antigen can comprise a pair of proteins selected from any pairs of one of ADAM-10, BCNP, CD9, EGFR, EpCam, IL1B, KLK2, MMP7, p53, PBP, PCSA, SERPINB3, SPDEF, SSX2, and SSX4; and one of EpCam or PCSA. The one or more microvesicle-associated antigen can comprise a pair of proteins selected from any pairs of EpCAM-EpCAM, EpCAM-KLK2, EpCAM-PBP, EpCAM-SPDEF, EpCAM-SSX2, EpCAM-SSX4, EpCAM-ADAM-10, EpCAM-SERPINB3, EpCAM-PCSA, EpCAM-p53, EpCAM-MMP7, EpCAM-IL1B, EpCAM-EGFR, EpCAM-CD9, EpCAM-BCNP, KLK2-EpCAM, KLK2-KLK2, KLK2-PBP, KLK2-SPDEF, KLK2-SSX2, KLK2-SSX4, KLK2-ADAM-10, KLK2-SERPINB3, KLK2-PCSA, KLK2-p53, KLK2-MMP7, KLK2-IL1B, KLK2-EGFR, KLK2-CD9, KLK2-BCNP, PBP-EpCAM, PBP-KLK2, PBP-PBP, PBP-SPDEF, PBP-SSX2, PBP-SSX4, PBP-ADAM-10, PBP-SERPINB3, PBP-PCSA, PBP-p53, PBP-MMP7, PBP-IL1B, PBP-EGFR, PBP-CD9, PBP-BCNP, SPDEF-EpCAM, SPDEF-KLK2, SPDEF-PBP, SPDEF-SPDEF, SPDEF-SSX2, SPDEF-SSX4, SPDEF-ADAM-10, SPDEF-SERPINB3, SPDEF-PCSA, SPDEF-p53, SPDEF-MMP7, SPDEF-IL1B, SPDEF-EGFR, SPDEF-CD9, SPDEF-BCNP, SSX2-EpCAM, SSX2-KLK2, SSX2-PBP, SSX2-SPDEF, SSX2-SSX2, SSX2-SSX4, SSX2-ADAM-10, SSX2-SERPINB3, SSX2-PCSA, SSX2-p53, SSX2-MMP7, SSX2-IL1B, SSX2-EGFR, SSX2-CD9, SSX2-BCNP, SSX4-EpCAM, SSX4-KLK2, SSX4-PBP, SSX4-SPDEF, SSX4-SSX2, SSX4-SSX4, SSX4-ADAM-10, SSX4-SERPINB3, SSX4-PCSA, SSX4-p53, SSX4-MMP7, SSX4-IL1B, SSX4-EGFR, SSX4-CD9, SSX4-BCNP, ADAM-10-EpCAM, ADAM-10-KLK2, ADAM-10-PBP, ADAM-10-SPDEF, ADAM-10-SSX2, ADAM-10-SSX4, ADAM-10-ADAM-10, ADAM-10-SERPINB3, ADAM-10-PCSA, ADAM-10-p53, ADAM-10-MMP7, ADAM-10-IL1B, ADAM-10-EGFR, ADAM-10-CD9, ADAM-10-BCNP, SERPINB3-EpCAM, SERPINB3-KLK2, SERPINB3-PBP, SERPINB3-SPDEF, SERPINB3-SSX2, SERPINB3-SSX4, SERPINB3-ADAM-10, SERPINB3-SERPINB3, SERPINB3-PCSA, SERPINB3-p53, SERPINB3-MMP7, SERPINB3-IL1B, SERPINB3-EGFR, SERPINB3-CD9, SERPINB3-BCNP, PCSA-EpCAM, PCSA-KLK2, PCSA-PBP, PCSA-SPDEF, PCSA-SSX2, PCSA-SSX4, PCSA-ADAM-10, PCSA-SERPINB3, PCSA-PCSA, PCSA-p53, PCSA-MMP7, PCSA-IL1B, PCSA-EGFR, PCSA-CD9, PCSA-BCNP, p53-EpCAM, p53-KLK2, p53-PBP, p53-SPDEF, p53-SSX2, p53-SSX4, p53-ADAM-10, p53-SERPINB3, p53-PCSA, p53-p53, p53-MMP7, p53-IL1B, p53-EGFR, p53-CD9, p53-BCNP, MMP7-EpCAM, MMP7-KLK2, MMP7-PBP, MMP7-SPDEF, MMP7-SSX2, MMP7-SSX4, MMP7-ADAM-10, MMP7-SERPINB3, MMP7-PCSA, MMP7-p53, MMP7-MMP7, MMP7-IL1B, MMP7-EGFR, MMP7-CD9, MMP7-BCNP, IL1B-EpCAM, IL1B-KLK2, IL1B-PBP, IL1B-SPDEF, IL1B-SSX2, IL1B-SSX4, IL1B-ADAM-10, IL1B-SERPINB3, IL1B-PCSA, IL1B-p53, IL1B-MMP7, IL1B-IL1B, IL1B-EGFR, IL1B-CD9, IL1B-BCNP, EGFR-EpCAM, EGFR-KLK2, EGFR-PBP, EGFR-SPDEF, EGFR-SSX2, EGFR-SSX4, EGFR-ADAM-10, EGFR-SERPINB3, EGFR-PCSA, EGFR-p53, EGFR-MMP7, EGFR-IL1B, EGFR-EGFR, EGFR-CD9, EGFR-BCNP, CD9-EpCAM, CD9-KLK2, CD9-PBP, CD9-SPDEF, CD9-SSX2, CD9-SSX4, CD9-ADAM-10, CD9-SERPINB3, CD9-PCSA, CD9-p53, CD9-MMP7, CD9-IL1B, CD9-EGFR, CD9-CD9, CD9-BCNP, BCNP-EpCAM, BCNP-KLK2, BCNP-PBP, BCNP-SPDEF, BCNP-SSX2, BCNP-SSX4, BCNP-ADAM-10, BCNP-SERPINB3, BCNP-PCSA, BCNP-p53, BCNP-MMP7, BCNP-IL1B, BCNP-EGFR, BCNP-CD9, BCNP-BCNP, and a combination thereof.

The one or more microvesicle-associated antigen can comprise a pair of proteins selected from any pairs of EpCAM and one of EpCAM, KLK2, PBP, SPDEF, SSX2, SSX4, and EGFR.

The one or more microvesicle-associated antigen can comprise a pair of proteins selected from any pairs of SSX4 and EpCAM; SSX4 and KLK2; SSX4 and PBP; SSX4 and SPDEF; SSX4 and SSX2; SSX4 and EGFR; SSX4 and MMP7; SSX4 and BCNP1; SSX4 and SERPINB3; KLK2 and EpCAM; KLK2 and PBP; KLK2 and SPDEF; KLK2 and SSX2; KLK2 and EGFR; KLK2 and MMP7; KLK2 and BCNP1; KLK2 and SERPINB3; PBP and EGFR; PBP and EpCAM; PBP and SPDEF; PBP and SSX2; PBP and SERPINB3; PBP and MMP7; PBP and BCNP1; EpCAM and SPDEF; EpCAM and SSX2; EpCAM and SERPINB3; EpCAM and EGFR; EpCAM and MMP7; EpCAM and BCNP1; SPDEF and SSX2; SPDEF and SERPINB3; SPDEF and EGFR; SPDEF and MMP7; SPDEF and BCNP1; SSX2 and EGFR; SSX2 and MMP7; SSX2 and BCNP1; SSX2 and SERPINB3; SERPINB3 and EGFR; SERPINB3 and MMP7; SERPINB3 and BCNP1; EGFR and MMP7; EGFR and BCNP1; MMP7 and BCNP1; and a combination thereof. The one or more microvesicle-associated antigen can comprise a pair of proteins selected from any pairs of EpCam-EpCam, EpCam-KLK2, EpCam-PBP, EpCam-SPDEF, EpCam-SSX2, EpCam-SSX4, EpCam-EGFR, and a combination thereof.

In some embodiments, the one or more microvesicle-associated antigen comprises a protein selected from the group consisting of EGFR, EpCAM, CD9, CD63, CD81, and a combination thereof. The one or more microvesicle-associated antigen can comprise MMP7.

Any of the microvesicle-associated antigen and pairs thereof may be used to detect microvesicles indicative of a cancer, including without limitation a prostate cancer.

In other embodiments of the methods herein, the one or more microvesicle-associated antigen comprises 5HT2B, 5T4 (trophoblast), ACO2, ACSL3, ACTN4, ADAM10, AGR2, AGR3, ALCAM, ALDH6A1, ANGPTL4, ANO9, AP1G1, APC, APEX1, APLP2, APP (Amyloid precursor protein), ARCN1, ARHGAP35, ARL3, ASAH1, ASPH (A-10), ATP1B1, ATP1B3, ATP5I, ATP5O, ATXN1, B7H3, BACE1, BAI3, BAIAP2, BCA-200, BDNF, BigH3, BIRC2, BLVRB, BRCA, BST2, C1GALT1, C1GALT1C1, C20orf3, CA125, CACYBP, Calmodulin, CAPN1, CAPNS1, CCDC64B, CCL2 (MCP-1), CCT3, CD10(BD), CD127 (IL7R), CD174, CD24, CD44, CD80, CD86, CDH1, CDH5, CEA, CFL2, CHCHD3, CHMP3, CHRDL2, CIB1, CKAP4, COPA, COX5B, CRABP2, CRIP1, CRISPLD1, CRMP-2, CRTAP, CTLA4, CUL3, CXCR3, CXCR4, CXCR6, CYB5B, CYB5R1, CYCS, CYFRA 21, DBI, DDX23, DDX39B, derlin 1, DHCR7, DHX9, DLD, DLL4, DNAJBL DPP6, DSTN, eCadherin, EEF1D, EEF2, EFTUD2, EIF4A2, EIF4A3, EpCaM, EphA2, ER(1) (ESR1), ER(2) (ESR2), Erb B4, Erb2, erb3 (Erb-B3), ERLIN2, ESD, FARSA, FASN, FEN1, FKBP5, FLNB, FOXP3, FUS, Gal3, GCDPF-15, GCNT2, GNAl2, GNG5, GNPTG, GPC6, GPD2, GPER (GPR30), GSPT1, H3F3B, H3F3C, HADH, HAP1, HER3, HIST1H1C, HIST1H2AB, HIST1H3A, HIST1H3C, HIST1H3D, HIST1H3E, HIST1H3F, HIST1H3G, HIST1H3H, HIST1H3I, HIST1H3J, HIST2H2BF, HIST2H3A, HIST2H3C, HIST2H3D, HIST3H3, HMGB1, HNRNPA2B1, HNRNPAB, HNRNPC, HNRNPD, HNRNPH2, HNRNPK, HNRNPL, HNRNPM, HNRNPU, HPS3, HSP-27, HSP70, HSP90B1, HSPA1A, HSPA2, HSPA9, HSPE1, IC3b, IDE, IDH3B, IDO1, IF130, IL1RL2, IL7, IL8, ILF2, ILF3, IQCG, ISOC2, IST1, ITGA7, ITGB7, junction plakoglobin, Keratin 15, KRAS, KRT19, KRT2, KRT7, KRT8, KRT9, KTN1, LAMP1, LMNA, LMNB1, LNPEP, LRPPRC, LRRC57, Mammaglobin, MAN1A1, MAN1A2, MART1, MATR3, MBD5, MCT2, MDH2, MFGE8, MFGE8, MGP, MMP9, MRP8, MUC1, MUC17, MUC2, MYO5B, MYOF, NAPA, NCAM, NCL, NG2 (CSPG4), Ngal, NHE-3, NME2, NONO, NPM1, NQO1, NT5E (CD73), ODC1, OPG, OPN (SC), OS9, p53, PACSIN3, PAICS, PARK7, PARVA, PC, PCNA, PCSA, PD-1, PD-L1, PD-L2, PGP9.5, PHB, PHB2, PIK3C2B, PKP3, PPL, PR(B), PRDX2, PRKCB, PRKCD, PRKDC, PSA, PSAP, PSMA, PSMB7, PSMD2, PSME3, PYCARD, RAB1A, RAB3D, RAB7A, RAGE, RBL2, RNPEP, RPL14, RPL27, RPL36, RPS25, RPS4X, RPS4Y1, RPS4Y2, RUVBL2, SET, SHMT2, SLAIN1, SLC39A14, SLC9A3R2, SMARCA4, SNRPD2, SNRPD3, SNX33, SNX9, SPEN, SPR, SQSTM1, SSBP1, ST3GAL1, STXBP4, SUB1, SUCLG2, Survivin, SYT9, TFF3 (secreted), TGOLN2, THBS1, TIMP1, TIMP2, TMED10, TMED4, TMED9, TMEM211, TOM1, TRAF4 (scaffolding), TRAIL-R2, TRAP1, TrkB, Tsg 101, TXNDC16, U2AF2, UEVLD, UFC1, UNC93a, USP14, VASP, VCP, VDAC1, VEGFA, VEGFR1, VEGFR2, VPS37C, WIZ, XRCC5, XRCC6, YB-1, YWHAZ, or any combination thereof. Vesicles carrying these markers may be used to detect microvesicles indicative of a cancer, including without limitation a breast cancer.

The one or more binding agent may comprise a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof. For example, the binding agent can be an antibody or an aptamer. The one or more binding agent can be used to capture and/or detect the one or more microvesicle. In an embodiment, the one or more binding agent binds to one or more surface antigen on the one or more microvesicle. The one or more surface antigen can comprise one or more protein.

In some embodiments, at least one of the one or more binding agent is tethered to a substrate. At at least one of the one or more binding agent can be labeled.

The one or more microvesicle may have a diameter between 10 nm and 2000 nm, e.g., between 20 nm and 200 nm.

Various techniques can be used to isolate the one or more microvesicle in whole or in part. For example, the one or more microvesicle can be subjected to size exclusion chromatography, density gradient centrifugation, differential centrifugation, nanomembrane ultrafiltration, immunoabsorbent capture, affinity purification, affinity capture, immunoassay, microfluidic separation, flow cytometry or combinations thereof.

The methods of the invention may further comprise detecting one or more payload biomarker within the one or more microvesicle. Microvesicle payload comprises one or more nucleic acid, peptide, protein, lipid, antigen, carbohydrate, and/or proteoglycan. The nucleic acid may comprise one or more DNA, mRNA, microRNA, snoRNA, snRNA, rRNA, tRNA, siRNA, hnRNA, or shRNA. In an embodiment, the one or more biomarker comprises payload within the one or more captured microvesicle. For example, the one or more biomarker can include mRNA payload. The one or more biomarker can also include microRNA payload. The one or more biomarker can also include protein payload, e.g., inner membrane protein or soluble protein.

In any of the various aspects of the invention, the detected presence or level the one or more microvesicle can be used to characterize a cancer. The concentration of the detected microvesicles can be compared to a reference in order to characterize the cancer. Any relevant phenotype of the cancer can be determined using the subject methods. For example, characterizing may comprise providing a prognostic, diagnostic or theranostic determination for the cancer, identifying the presence or risk of the cancer, or identifying the cancer as metastatic or aggressive.

Any appropriate cancer can be assessed using the subject methods. For example, the cancer may comprise an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. In some embodiments, the cancer comprises prostate cancer. In some embodiments, the cancer comprises breast cancer.

The methods of the invention can be performed in vitro, e.g., using an in vitro biological sample or a cell culture sample.

The invention provides use of one or more reagent to carry out the methods herein. Similarly, the invention contemplates use of a reagent for the manufacture of a kit or reagent for carrying out the methods herein. The invention also provides a kit comprising one or more reagent to carry out the methods herein. For any of the uses or kits of the invention, the one or more reagent and be selected from the group consisting of one or more reagent capable of binding to a microvesicle surface antigen, one or more lipophilic dye or precursor thereof, an affinity column to remove one or more abundant protein, a reagent to precipitate one or more abundant protein, a dilution buffer, one or more population of microvesicles, and a combination thereof.

In an aspect, the invention provides an aptamer that comprises a first binding region to a first target, a second binding region to a second target, and a linker region between the first binding region and the second binding region.

The first target may comprise a cancer or cell-of-origin specific protein marker. The first target can include a microvesicle surface antigen. In some embodiments, the first target is selected from any of Table 3, Table 4 or Table 5 herein. For example, the first target can be selected from the group consisting of 5T4, A33, ACTG1, ADAM10, ADAM15, AFP, ALA, ALDOA, ALIX, ALP, ALX4, ANCA, Annexin V, ANXA2, ANXA6, APC, APOA1, ASCA, ASPH, ATP1A1, AURKA, AURKB, B7H3, B7H4, BANK1, BASP1, BCA-225, BCNP1, BDNF, BRCA, C1orf58, C20orf114, C8B, CA125 (MUC16), CA-19-9, CAPZA1, CAV1, C-Bir, CCSA-2, CCSA-3&4, CD1.1, CD10, CD151, CD174 (Lewis y), CD24, CD2AP, CD37, CD44, CD46, CD53, CD59, CD63, CD66 CEA, CD73, CD81, CD82, CD9, CDA, CDAC1 1a2, CEA, C-Erbb2, CFL1, CFP, CHMP4B, CLTC, COTL1, CRMP-2, CRP, CRTN, CTNND1, CTSB, CTSZ, CXCL12, CYCS, CYFRA21-1, DcR3, DLL4, DPP4, DR3, EEF1A1, EGFR, EHD1, ENO1, EpCAM, EphA2, ER, ErbB4, EZH2, F11R, F2, F5, FAM125A, FASL, Ferritin, FNBP1L, FOLH1, FRT, GAL3, GAPDH, GDF15, GLB1, GPCR (GPR110), GPR30, GPX3, GRO-1, Gro-alpha, HAP, HBD 1, HBD2, HER 3 (ErbB3), HIST1H1C, HIST1H2AB, HNP1-3, HSP, HSP70, HSP90AB1, HSPA1B, HSPA8, hVEGFR2, iC3b, ICAM, IGSF8, IL6, IL-1B, IL6R, IL8, IMP3, INSIG 2, ITGB1, ITIH3, JUP, KLK2, L1CAM, LAMN, LDH, LDHA, LDHB, LUM, LYZ, MACC-1, MAPK4, MART-1, MCP-1, M-CSF, MFGE8, MGAM, MGC20553, MIC1, MIF, MIS RII, MMG, MMP26, MMP7, MMP9, MS4A1, MUC1, MUC17, MUC2, MYH2, MYL6B, Ncam, NGAL, NME1, NME2, NNMT, NPGP/NPFF2, OPG, OPG-13, OPN, p53, PA2G4, PABPC1, PABPC4, PACSIN2, PBP, PCBP2, PCSA, PDCD6IP, PDGFRB, PGP9.5, PIM1, PR (B), PRDX2, PRL, PSA, PSCA, PSMA, PSMA1, PSMA2, PSMA4, PSMA6, PSMA7, PSMB1, PSMB2, PSMB3, PSMB4, PSMB5, PSMB6, PSMB8, PSME3, PTEN, PTGFRN, Rab-5b, Reg IV, RPS27A, RUNX2, SCRN1, SDCBP, seprase, Sept-9, SERINC5, SERPINB3, SERPINB3, SH3GL1, SLC3A2, SMPDL3B, SNX9, SPARC, SPB, SPDEF, SPON2, SPR, SRVN, SSX2, SSX4, STAT 3, STEAP, STEAP1, TACSTD1, TCN2, tetraspanin, TF (FL-295), TFF3, TGM2, THBS1, TIMP, TIMP1, TIMP2, TMEM211, TMPRSS2, TNF-alpha, TPA, TPI1, TPS, Trail-R2, Trail-R4, TrKB, TROP2, TROP2, Tsg 101, TUBB, TWEAK, UNC93A, VDAC2, VEGF A, VPS37B, YPSMA-1, YWHAG, YWHAQ, and YWHAZ. The first target can include a protein selected from the group consisting of 5HT2B, 5T4 (trophoblast), ACO2, ACSL3, ACTN4, ADAM10, AGR2, AGR3, ALCAM, ALDH6A1, ANGPTL4, ANO9, AP1G1, APC, APEX1, APLP2, APP (Amyloid precursor protein), ARCN1, ARHGAP35, ARL3, ASAH1, ASPH (A-10), ATP1B1, ATP1B3, ATP5I, ATP5O, ATXN1, B7H3, BACE1, BAI3, BAIAP2, BCA-200, BDNF, BigH3, BIRC2, BLVRB, BRCA, BST2, C1GALT1, C1GALT1C1, C20orf3, CA125, CACYBP, Calmodulin, CAPN1, CAPNS1, CCDC64B, CCL2 (MCP-1), CCT3, CD10(BD), CD127 (IL7R), CD174, CD24, CD44, CD80, CD86, CDH1, CDH5, CEA, CFL2, CHCHD3, CHMP3, CHRDL2, CIB1, CKAP4, COPA, COX5B, CRABP2, CRIP1, CRISPLD1, CRMP-2, CRTAP, CTLA4, CUL3, CXCR3, CXCR4, CXCR6, CYB5B, CYB5R1, CYCS, CYFRA 21, DBI, DDX23, DDX39B, derlin 1, DHCR7, DHX9, DLD, DLL4, DNAJB1, DPP6, DSTN, eCadherin, EEF1D, EEF2, EFTUD2, EIF4A2, EIF4A3, EpCaM, EphA2, ER(1) (ESR1), ER(2) (ESR2), Erb B4, Erb2, erb3 (Erb-B3?), ERLIN2, ESD, FARSA, FASN, FEN1, FKBP5, FLNB, FOXP3, FUS, Gal3, GCDPF-15, GCNT2, GNAl2, GNG5, GNPTG, GPC6, GPD2, GPER (GPR30), GSPT1, H3F3B, H3F3C, HADH, HAP1, HER3, HIST1H1C, HIST1H2AB, HIST1H3A, HIST1H3C, HIST1H3D, HIST1H3E, HIST1H3F, HIST1H3G, HIST1H3H, HIST1H3I, HIST1H3J, HIST2H2BF, HIST2H3A, HIST2H3C, HIST2H3D, HIST3H3, HMGB1, HNRNPA2B1, HNRNPAB, HNRNPC, HNRNPD, HNRNPH2, HNRNPK, HNRNPL, HNRNPM, HNRNPU, HPS3, HSP-27, HSP70, HSP90B1, HSPA1A, HSPA2, HSPA9, HSPE1, IC3b, IDE, IDH3B, IDO1, IFI30, IL1RL2, IL7, IL8, ILF2, ILF3, IQCG, ISOC2, IST1, ITGA7, ITGB7, junction plakoglobin, Keratin 15, KRAS, KRT19, KRT2, KRT7, KRT8, KRT9, KTN1, LAMP1, LMNA, LMNB1, LNPEP, LRPPRC, LRRC57, Mammaglobin, MAN1A1, MAN1A2, MART1, MATR3, MBD5, MCT2, MDH2, MFGE8, MFGE8, MGP, MMP9, MRP8, MUC1, MUC17, MUC2, MYO5B, MYOF, NAPA, NCAM, NCL, NG2 (CSPG4), Ngal, NHE-3, NME2, NONO, NPM1, NQO1, NT5E (CD73), ODC1, OPG, OPN (SC), OS9, p53, PACSIN3, PAICS, PARK7, PARVA, PC, PCNA, PCSA, PD-1, PD-L1, PD-L2, PGP9.5, PHB, PHB2, PIK3C2B, PKP3, PPL, PR(B), PRDX2, PRKCB, PRKCD, PRKDC, PSA, PSAP, PSMA, PSMB7, PSMD2, PSME3, PYCARD, RAB1A, RAB3D, RAB7A, RAGE, RBL2, RNPEP, RPL14, RPL27, RPL36, RPS25, RPS4X, RPS4Y1, RPS4Y2, RUVBL2, SET, SHMT2, SLAIN1, SLC39A14, SLC9A3R2, SMARCA4, SNRPD2, SNRPD3, SNX33, SNX9, SPEN, SPR, SQSTM1, SSBP1, ST3GAL1, STXBP4, SUB1, SUCLG2, Survivin, SYT9, TFF3 (secreted), TGOLN2, THBS1, TIMP1, TIMP2, TMED10, TMED4, TMED9, TMEM211, TOM1, TRAF4 (scaffolding), TRAIL-R2, TRAP1, TrkB, Tsg 101, TXNDC16, U2AF2, UEVLD, UFC1, UNC93a, USP14, VASP, VCP, VDAC1, VEGFA, VEGFR1, VEGFR2, VPS37C, WIZ, XRCC5, XRCC6, YB-1, YWHAZ, or any combination thereof. In some embodiments, the first target is a cancer biomarker selected from the group consisting of p53, p63, p73, mdm-2, procathepsin-D, B23, C23, PLAP, CA125, MUC-1, HER2, NY-ESO-1, SCP1, SSX-1, SSX-2, SSX-4, HSP27, HSP60, HSP90, GRP78, TAG72, HoxA7, HoxB7, EpCAM, B7H3, ras, mesothelin, survivin, EGFK, MUC-1, or c-myc.

In some embodiments, the second target of the subject aptamer comprises an immunosuppressive protein. For example, the second target can be selected from the group consisting of TGF-β, CD39, CD73, IL10, FasL or TRAIL. The second target can also be selected from the group consisting of FasL, programmed cell death 1 (PD-1), programmed death ligand-1 (PD-L1; B7-H1), programmed death ligand-2 (PD-L2; B7-DC), B7-H3, and B7-H4.

The linker region of the subject aptamer may comprise an immune-modulatory oligonucleotide sequence. In some embodiments, the linker region comprises an immunostimulatory sequence. For example, the linker region may comprise one or more CpG motif. The CpG region can be at least 50, 55, 60, 65, 70, 75, 80, 85, 90, 95, 96, 97, 98, 99 or 100 percent homologous to one or more of SEQ ID NOs. 2-4, or a functional fragment thereof.

The linker region of the subject aptamer may comprise an anti-proliferative or pro-apoptotic sequence. For example, the linker region may comprise a polyG sequence. The polyG region may be at least 50, 55, 60, 65, 70, 75, 80, 85, 90, 95, 96, 97, 98, 99 or 100 percent homologous to one or more of SEQ ID NOs. 5-10, or a functional fragment thereof.

As desired, the linker region of the aptamer comprises an immunostimulatory and an anti-proliferative or pro-apoptotic sequence. For example, the linker region can comprise a hybrid CpG-polyG region that is at least 50, 55, 60, 65, 70, 75, 80, 85, 90, 95, 96, 97, 98, 99 or 100 percent homologous to one or more of SEQ ID NOs. 11-28, or a functional fragment thereof.

The aptamer of the invention can be modified to comprise at least one chemical modification. The modification can be selected from the group consisting: of a chemical substitution at a sugar position; a chemical substitution at a phosphate position; and a chemical substitution at a base position of the nucleic acid. In some embodiments, the modification is selected from the group consisting of: incorporation of a modified nucleotide, 3′ capping, conjugation to an amine linker, conjugation to a high molecular weight, non-immunogenic compound, conjugation to a lipophilic compound, conjugation to a drug, conjugation to a cytotoxic moiety and labeling with a radioisotope. The non-immunogenic, high molecular weight compound can be a polyalkylene glycol, e.g., polyethylene glycol.

The aptamer of the invention can further comprise additional elements to add desired biological effects. For example, the aptamer may comprise an immunostimulatory moiety. In other embodiments, the aptamer may comprise a membrane disruptive moiety. For example, the aptamer may comprise an oligonucleotide sequence including without limitation Toll-Like Receptor (TLR) agonists like CpG sequences which are immunostimulatory and/or polyG sequences which can be anti-proliferative or pro-apoptotic. The aptamer may also be conjugated to one or more chemical moiety that provides such effects. For example, the aptamer may be conjugated to a detergent like moiety to disrupt the membrane of the target vesicle. Useful ionic detergents include sodium dodecyl sulfate (SDS, sodium lauryl sulfate (SLS)), sodium laureth sulfate (SLS, sodium lauryl ether sulfate (SLES)), ammonium lauryl sulfate (ALS), cetrimonium bromide, cetrimonium chloride, cetrimonium stearate, and the like. Useful non-ionic (zwitterionic) detergents include polyoxyethylene glycols, polysorbate 20 (also known as Tween 20), other polysorbates (e.g., 40, 60, 65, 80, etc), Triton-X (e.g., X100, X114), 3-[(3-cholamidopropyl)dimethylammonio]-1-propanesulfonate (CHAPS), CHAPSO, deoxycholic acid, sodium deoxycholate, NP-40, glycosides, octyl-thio-glucosides, maltosides, and the like. The moiety can be vaccine like moiety or antigen that stimulates an immune response. In an embodiment, the immune stimulating moiety comprises a superantigen. In some embodiments, the superantigen can be selected from the group consisting of staphylococcal enterotoxins (SEs), a Streptococcus pyogenes exotoxin (SPE), a Staphylococcus aureus toxic shock-syndrome toxin (TSST-1), a streptococcal mitogenic exotoxin (SME), a streptococcal superantigen (SSA), a hepatitis surface antigen, or a combination thereof. Other bacterial antigens that can be used with the invention comprise bacterial antigens such as Freund's complete adjuvant, Freund's incomplete adjuvant, monophosphoryl-lipid A/trehalose dicorynomycolate (Ribi's adjuvant), BCG (Calmette-Guerin Bacillus; Mycobacterium bovis), and Corynebacterium parvum. The immune stimulating moiety can also be a non-specific immunostimulant, such as an adjuvant or other non-specific immunostimulator. Useful adjuvants comprise without limitation aluminium salts, alum, aluminium phosphate, aluminium hydroxide, squalene, oils, MF59, and AS03 (“Adjuvant System 03”). The adjuvant can be selected from the group consisting of Cationic liposome-DNA complex JVRS-100, aluminum hydroxide vaccine adjuvant, aluminum phosphate vaccine adjuvant, aluminum potassium sulfate adjuvant, Alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, ASO4, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and Matrix-S. Additional adjuvants that can be used with the aptamers of the invention can be identified using the Vaxjo database. See Sayers S, Ulysse G, Xiang Z, and He Y. Vaxjo: a web-based vaccine adjuvant database and its application for analysis of vaccine adjuvants and their uses in vaccine development. Journal of Biomedicine and Biotechnology. 2012; 2012:831486. Epub 2012 Mar. 13. PMID: 22505817; www.violinet.org/vaxjo/. Other useful non-specific immunostimulators comprise histamine, interferon, transfer factor, tuftsin, interleukin-1, female sex hormones, prolactin, growth hormone vitamin D, deoxycholic acid (DCA), tetrachlorodecaoxide (TCDO), and imiquimod or resiquimod, which are drugs that activate immune cells through the toll-like receptor 7. One of skill will appreciate that functional fragments of the immunomodulating and/or membrance disruptive moieties can be covalently or non-covalently attached to the aptamer.

In a related aspect, the invention provides a pharmaceutical composition comprising a therapeutically effective amount of the aptamer above, or a salt thereof, and a pharmaceutically acceptable carrier or diluent. In still another related aspect, the invention provides a method of treating or ameliorating a disease associated with a neoplastic growth, comprising administering the pharmaceutical composition to a patient in need thereof. In some embodiments, the pharmaceutical composition and method of use are used to treat a cancer patient. The cancer may comprise one or more of an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

The invention further provides a kit comprising one or more aptamer as described above, or a pharmaceutical composition thereof. The invention also provides a kit comprising a reagent for carrying out the method of treatment above, as well as use of a reagent for carrying out the method. In various embodiments, the invention provides use of a reagent for the manufacture of a kit or reagent for carrying out the method, and for the manufacture of a medicament for carrying out the method of treatment. The reagent in the kit or use may comprise an aptamer as described herein, or a pharmaceutical composition thereof.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

FIG. 1A depicts a method of identifying a biosignature comprising nucleic acid to characterize a phenotype. FIG. 1B depicts a method of identifying a biosignature of a vesicle or vesicle population to characterize a phenotype.

FIGS. 2A-2G illustrate methods of assessing biomarkers such as microvesicle surface antigens. FIG. 2A is a schematic of a planar substrate coated with a capture agent, such as an aptamer or antibody, which captures vesicles expressing the target antigen of the capture agent. The capture agent may bind a protein expressed on the surface of vesicles shed from diseased cells (“disease vesicle”). The detection agent, which may also be an aptamer or antibody, carries a detectable label, here a fluorescent signal. The detection agent binds to the captured vesicle and provides a detectable signal via its fluorescent label. The detection agent can detect an antigen that is generally associated with vesicles, or is associated with a cell-of-origin or a disease, e.g., a cancer. FIG. 2B is a schematic of a particle bead conjugated with a capture agent, which captures vesicles expressing the target antigen of the capture agent. The capture agent may bind a protein expressed on the surface of vesicles shed from diseased cells (“disease vesicle”). The detection agent, which may also be an aptamer or antibody, carries a detectable label, here a fluorescent signal. The detection agent binds to the captured vesicle and provides a detectable signal via its fluorescent label. The detection agent can detect an antigen that is generally associated with vesicles, or is associated with a cell-of-origin or a disease, e.g., a cancer. FIG. 2C is an example of a screening scheme that can be performed by using different combinations of capture and detection agents to the indicated biomarkers. The biomarker combinations can be detected using assays as shown in FIGS. 2A-2B. FIGS. 2D-2E present illustrative schemes for capturing and detecting vesicles to characterize a phenotype. FIG. 2F presents illustrative schemes for assessing vesicle payload to characterize a phenotype. FIG. 2G presents illustrative schemes for capturing and detecting vesicles and optionally assessing payload to characterize a phenotype. FIG. 2H presents illustrative schemes for using a lipid dye to detect vesicles and characterize a phenotype.

FIG. 3 illustrates a computer system that can be used in some exemplary embodiments of the invention.

FIG. 4 illustrates a method of depicting results using a bead based method of detecting vesicles from a subject. The number of beads captured at a given intensity is an indication of how frequently a vesicle expresses the detection protein at that intensity. The more intense the signal for a given bead, the greater the expression of the detection protein. The figure shows a normalized graph obtained by combining normal patients into one curve and cancer patients into another, and using bio-statistical analysis to differentiate the curves. Data from each individual is normalized to account for variation in the number of beads read by the detection machine, added together, and then normalized again to account for the different number of samples in each population.

FIG. 5 illustrates the capture of prostate cancer cells-derived vesicles from plasma with EpCam by assessing TMPRSS2-ERG expression. VCaP purified vesicles were spiked into normal plasma and then incubated with Dynal magnetic beads coated with either the EpCam or isotype control antibody. RNA was isolated directly from the Dynal beads. Equal volumes of RNA from each sample were used for RT-PCR and subsequent Taqman assays.

FIG. 6 depicts a bar graph of miR-21 or miR-141 expression with CD9 bead capture. 1 ml of plasma from prostate cancer patients, 250 ng/ml of LNCaP, or normal purified vesicles were incubated with CD9 coated Dynal beads. The RNA was isolated from the beads and the bead supernatant. One sample (#6) was also uncaptured for comparison. microRNA expression was measured with qRT-PCR and the mean CT values for each sample compared. CD9 capture improves the detection of miR-21 and miR-141 in prostate cancer samples.

FIG. 7A illustrates separation and identification of vesicles using the MoFlo XDP. FIG. 7B illustrates FACS analysis of VCaP cells and exosomes stained with antibodies to CD9, B7H3, PCSA and PSMA. FIG. 7C illustrates different patterns of miR expression were obtained in flow sorted B7H3+ or PSMA+ vesicle populations as compared to overall vesicle population.

FIGS. 8A-H illustrates detecting vesicles in a sample. FIG. 8A represents a schematic of isolating vesicles from plasma using a column based filtering method, wherein the isolated vesicles are subsequently assessed. FIG. 8B represents a schematic of compression of a membrane of a vesicle due to high-speed centrifugation, such as ultracentrifugation. FIG. 8C represents a schematic of detecting vesicles bound to microspheres using laser detection. FIG. 8D represents an example of detecting prostate derived vesicles bound to a substrate. The microvesicles are captured with capture agents specific to PCSA, PSMA or B7H3 tethered to the substrate. The so-captured vesicles are labeled with fluorescently labeled detection agents specific to CD9, CD63 and CD81. FIG. 8E illustrates correlation of CD9 positive vesicles detected using a microsphere platform (Y-axis) or flow cytometry (X-axis). To calculate median fluorescence intensity (MFIs), vesicles were captured with anti-CD9 antibodies tethered to microspheres and detected using fluorescently labeled detection antibodies specific to CD9, CD63 and CD81. FIG. 8F illustrates correlation of PSMA, PCSA or B7H3 positive vesicles detected using a microsphere platform (Y-axis) or BCA protein assay (X-axis). To calculate MFIs, vesicles were captured with antibodies to B7H3, PSMA or PCSA tethered to microspheres and detected using fluorescently labeled detection antibodies specific to CD9, CD63 and CD81. FIG. 8G illustrates similar performance for detecting CD81 positive vesicles using a microsphere assay in a single-plex or multi-plex fashion. Vesicles were captured with anti-CD81 antibodies tethered to microspheres and detected using fluorescently labeled detection antibodies specific to CD9, CD63 and CD81. FIG. 8H illustrates similar performance for detecting B7H3, CD63, CD9 or EpCam positive vesicles using a microsphere assay in a single-plea or multi-plea fashion. Vesicles were captured with antibodies to B7H3, CD63, CD9 or EpCam tethered to microspheres and detected using fluorescently labeled detection antibodies specific to CD9, CD63 and CD81.

FIG. 9A illustrates the ability of a vesicle bio-signature to discriminate between normal prostate and PCa samples. Cancer markers included EpCam and B7H3. General vesicle markers included CD9, CD81 and CD63. Prostate specific markers included PCSA. PSMA can be used as well as PCSA. The test was found to be 98% sensitive and 95% specific for PCa vs normal samples. FIG. 9B illustrates mean fluorescence intensity (MFI) on the Y axis for vesicle markers of FIG. 9A in normal and prostate cancer patients.

FIG. 10 is a schematic for a decision tree for a vesicle prostate cancer assay for determining whether a sample is positive for prostate cancer.

FIG. 11 shows the results of a vesicle detection assay for prostate cancer following the decision tree versus detection using elevated PSA levels.

FIG. 12 illustrates levels of miR-145 in vesicles isolated from control and PCa samples.

FIGS. 13A-13E illustrate the use of microRNA to identify false negatives from a vesicle-based diagnostic assay for prostate cancer. FIG. 13A illustrates a scheme for using miR analysis within vesicles to convert false negatives into true positives, thereby improving sensitivity. FIG. 13B illustrates a scheme for using miR analysis within vesicles to convert false positives into true negatives, thereby improving specificity. Normalized levels of miR-107 (FIG. 13C) and miR-141 (FIG. 13D) are shown on the Y axis for true positives (TP) called by the vesicle diagnostic assay, true negatives (TN) called by the vesicle diagnostic assay, false positives (FP) called by the vesicle diagnostic assay, and false negatives (FN) called by the vesicle diagnostic assay. miR-107 and miR-141 can be used in the schematic shown in FIG. 13A and FIG. 13B. FIG. 13E shows Taqman qRT-PCR verification of increased miR-107 in plasma cMVs of prostate cancer patients compared to patients without prostate cancer using a different sample cohort.

FIGS. 14A-D illustrate KRAS sequencing in a colorectal cancer (CRC) cell line and patient sample. Samples comprise genomic DNA obtained from the cell line (FIG. 14B) or from a tissue sample from the patient (FIG. 14D), or cDNA obtained from RNA payload within vesicles shed from the cell line (FIG. 14A) or from a plasma sample from the patient (FIG. 14C).

FIGS. 15A-B illustrate immunoprecipitation of microRNA from human plasma. FIG. 15A shows the mean quantity of miR-16 detected in various fractions of human plasma. “Beads” are the amount of miR-16 that co-immunoprecipitated using antibodies to Argonaute2 (Ago2), Apolipoprotein A1 (ApoA1), GW182, and an IgG control. “Dyna” refers to immunoprecipitation using Dynabead Protein G, whereas “Magna” refers to Magnabind Protein G beads. “Supernt” are the amount of miR-16 detected in the supernatant of the immunoprecipitation reactions. See Examples for details. FIG. 15B is the same as FIG. 15A except that miR-92a was detected.

FIG. 16 illustrates flow sorting of complexes stained with PE labeled anti-PCSA antibodies and FITC labeled anti-Ago2 antibodies.

FIGS. 17A-D illustrate detection of microRNA in PCSA/Ago2 positive complexes in human plasma samples. The plasma samples were from subjects with prostate cancer (PrC) or normal controls (normal). FIG. 17A shows miR-22 copy number in total circulating microvesicle population from human plasma. FIG. 17B shows plasma-derived complexes were sorted using antibodies against PCSA and Argonaute 2 (Ago2). RNA was isolated and the copy number of miR-22 was determined in the population of PCSA/Ago2 double positive events. FIG. 17C shows the number of PCSA/Ago2 double positive events counted by flow cytometry for each plasma sample. FIG. 17D shows copy number of miR-22 divided by the total number of PCSA/Ago2 positive events for each plasma sample. This yields the copy number of miR-22 per PCSA/Ago2 double positive complex.

FIGS. 18A-F illustrate dot plots of raw background subtracted fluorescence values of selected mRNAs from microarray profiling of vesicle mRNA payload levels. In each plot, the Y axis shows raw background subtracted fluorescence values (Raw BGsub Florescence). The X axis shows dot plots for four normal control plasmas and four plasmas from prostate cancer patients. The mRNAs shown are A2ML1 (FIG. 18A), GABARAPL2 (FIG. 18B), PTMA (FIG. 18C), RABAC1 (FIG. 18D), SOX1 (FIG. 18E), and ETFB (FIG. 18F).

FIGS. 19A-E illustrate a microRNA functional assay. FIG. 19A shows a labeled synthetic RNA molecule 191-196 and a ribonucleoprotein complex containing a target microRNA 197 of interest. FIG. 19B demonstrates cleavage of the synthetic RNA molecule at the target recognition site 193 when recognized by the ribonucleoprotein complex 197, thereby releasing the label 195-196. FIGS. 19C-E illustrate input ribonucleoprotein complex from various sources.

FIGS. 20A-F show ROC curves demonstrating the ability of 3-marker panel vesicle capture and detection agents to distinguish prostate cancer. Illustrative results for distinguishing prostate cancer (PCa+) samples from all other samples (PCA−) (see Table 26) using 3-marker combinations are shown. The dark grey line (more jagged line to the left) corresponds to resubstitution performance and the smoother black line was generated using 10-fold cross-validation. ROC curves are shown generated using diagonal linear discriminant analysis (FIG. 20A; resubstitution AUC=0.87; cross validation AUC=0.86), linear discriminant analysis (FIG. 20B; resubstitution AUC=0.87; cross validation AUC=0.86), support vector machine (FIG. 20C; resubstitution AUC=0.87; cross validation AUC=0.86), tree-based gradient boosting (FIG. 20D; resubstitution AUC=0.89; cross validation AUC=0.84), lasso (FIG. 20E; resubstitution AUC=0.87; cross validation AUC=0.86), and neural network (FIG. 20F; resubstitution AUC=0.87; cross validation AUC=0.72).

FIGS. 21A-C illustrate the performance of a three marker panel consisting of the following markers: 1) Epcam detector-MMP7 capture; 2) PCSA detector-MMP7 capture; 3) Epcam detector-BCNP capture. The sample cohort was a restricted set wherein patients were age<75, serum PSA<10 ng/ml and no previous biopsy (N=127). An ROC curve generated using a diagonal linear discriminant analysis in this setting is shown in FIG. 21A. In the figure, the arrow indicates the threshold point along the curve where sensitivity equals 90% and specificity equals 80%. Another view of this threshold is shown in FIG. 21B, which shows the distribution of PCA+ and PCA− samples falling on either side of the indicated threshold line. The individual contribution of the Epcam detector-MMP7 capture marker is shown in FIG. 21C. “PCA, Current Biopsy” refers to men who had a first positive biopsy, whereas “PCA, Previous Biopsy” refers to the watchful waiting cohort.

FIGS. 22A-B show ROC curves demonstrating the ability of different vesicle capture and detection agents to distinguish prostate cancer. The performance of a 5-marker panel was determined in two settings using a linear discriminant analysis and 10-fold cross-validation or re-substitution methodology. ROC curves for the Model A setting (i.e., all PCa versus all other patient samples) are shown in FIG. 22A. The marker panel in this setting consisted of: 1) Epcam detector-MMP7 capture; 2) PCSA detector-MMP7 capture; 3) Epcam detector-BCNP capture; 4) PCSA detector-Adam10 capture; and 5) PCSA detector-KLK2 capture. In FIG. 22A, the upper more jagged line corresponds to the re-substitution method. The AUC was 0.90. Using cross-validation, the calculated AUC was 0.87. At the point indicated by the solid arrow, the model using cross-validation achieved 92% sensitivity and 50% specificity. At the point indicated by the solid arrow, the model using cross-validation achieved 82% sensitivity and 80% specificity. ROC curves for the Model C setting (i.e., restricted sample set as described below for Table 30) are shown in FIG. 22B. The marker panel in this setting consisted of: 1) Epcam detector-MMP7 capture; 2) PCSA detector-MMP7 capture; 3) Epcam detector-BCNP capture; 4) PCSA detector-Adam10 capture; and 5) CD81 detector-MMP7 capture. In FIG. 22B, the upper more jagged line corresponds to the re-substitution method. The AUC was 0.91. Using cross-validation, the calculated AUC was 0.89. At the point indicated by the arrow, the cross-validation model achieved 95% sensitivity and 60% specificity.

FIGS. 23A-D shows levels of microRNA species in PCSA+ circulating microvesicles from the plasma of men with prostate cancer and benign prostate disorders. In FIG. 23A, the Ct from the Exiqon cards for miR-1974 (which overlaps a mitochondrial tRNA) is shown in the various pools. The prostate cancer samples had higher levels of this miR than other samples. FIG. 23B shows the copy number of the miR in the pools as measured by taqman analysis using an ABI 7900. In FIG. 23C, the Ct from the Exiqon cards for miR-320b is shown in the various pools. The prostate cancer samples had lower levels of this miR than other samples. FIG. 23D shows the copy number of miR-320b in the pools as measured by taqman analysis using an ABI 7900.

FIG. 24 shows detection of a standard curve for a synthetic miR16 standard (10̂6-10̂1) and detection of miR16 in triplicate from a human plasma sample. As indicated by the legend, the data was taken from a Fluidigm Biomark (Fluidigm Corporation, South San Francisco, Calif.) using 48.48 Dynamic Array™ IFCs, 96.96 Dynamic Array™ IFCs, or with an ABI 7900HT Taqman assay (Applied Biosystems, Foster City, Calif.). All levels were determined under multiplex conditions.

FIGS. 25A-G show levels of alkaline phosphatase (intestinal) (FIG. 25A), CD-56 (FIG. 25B), CD-3 zeta (FIG. 25C), map1b (FIG. 25D), 14.3.3 pan (FIG. 25E), filamin (FIG. 25F), and thrombospondin (FIG. 25G) associated with microvesicles from plasma of normal (non-cancer) control individuals, breast cancer patients, brain cancer patients, lung cancer patients, colorectal cancer patients, colon adenoma patients, BPH patients (benign), inflamed prostate patients (inflammation), HGPIN patients, and prostate cancer patients, as indicated in the figures. Vesicles were concentrated then incubated with antibody arrays. Vesicles bound to antibodies to various proteins were fluorescently detected.

FIG. 26A illustrates a protein gel demonstrating removal of HSA and antibody heavy and light chains in the indicated samples. The columns in the gel are as follows: “Raw” (Plasma without any treatment); “Conc” (Plasma concentrated via nanomembrane filtration); “FTp” (Plasma flow through from treatment with Pierce Albumin and IgG Removal Kit, Thermo Fisher Scientific Inc., Rockford, Ill. USA); “FTv” (Plasma flow through from treatment with Vivapure® Anti-HSA/IgG Kit from Sartorius Stedim North America Inc., Edgewood, N.Y. USA); “IgG” (IgG control); “M” (molecular weight marker). FIG. 26B shows an example using the protocol to detect microvesicles. The cMVs were detected using Anti-MMP7-FITC antibody conjugate (Millipore anti-MMP7 monoclonal antibody 7B2). The plot shows the frequency of events detected versus concentration of the detection antibody. FIG. 26C shows EpCam expression in human serum albumin (HSA) depleted plasma sample. The x-axis refers to concentration of EpCam+ vesicles in various aliquots. The Y axis illustrates median fluorescent intensity (MFI) detected in a microbead assay using PE labeled anti-EpCAM antibodies to detect the vesicles. “Isotype” refers to detection using PE anti-IgG antibodies as a control. FIG. 26D is similar to FIG. 26C except that PE-labeled anti-MMP7 antibodies were used to detect the microvesicles. Shown are samples that were pre-treated to remove HSA (“HSA depleted”) or not (“HSA non-depleted”). “iso” refers to the anti-IgG antibody controls. FIG. 26E illustrates detection of vesicles in plasma after treatment with thromboplastin to precipitate fibrinogen. The Y axis illustrates median fluorescent intensity (MFI) detected in a microbead assay using bead-conjugated anti-KLK2 to capture the vesicles and a PE labeled anti-EpCAM aptamer to detect the vesicles. The X-axis groups 4 plasma samples (cancer sample C1, cancer sample C2, benign sample B1, benign sample B2) into 6 experimental conditions, V1-V6. As indicated by the thromboplastin incubation time and concentration below the plot, the thromboplastin treatment stringency increased from V1-V6.

FIGS. 27A-D illustrate the use of an anti-EpCAM aptamer (Aptamer 4; SEQ ID NO. 1) to detect a microvesicle population. Vesicles in patient plasma samples were captured using bead-conjugated antibodies to the indicated microvesicle surface antigens (FIG. 27A: EGFR; FIG. 27B: PBP; FIG. 27C: EpCAM; FIG. 27D: KLK2). Fluorescently labeled Aptamer 4 was used as a detector in the microbead assay. The figure shows average median fluorescence values (MFI values) for three prostate cancer (C1-C3) and three normal samples (N1-N3) in each plot. In each plot, the samples from left to right are ordered as: C1, C2, C3, N1, N2, N3.

FIGS. 28A-G illustrate presence of transcription factors in circulating microvesicles from cancer patients. STAT3 expression was determined for VCaP-derived cMVs (FIG. 28A and FIG. 28B) or cMVs from patient plasma (FIG. 28C and FIG. 28D) and co-stained for CD9 expression. cMVs were permeabilized using Life Technologies' Fix and Perm® cell fixation and permeabilization kit without washing steps and analyzed using a Beckman Coulter MoFlo XDP flow cytometer. FIGS. 28A-D indicate the percentage of double stained (STAT3+/CD9+) events in the upper right quadrant. To evaluate transcription factor expression in multiplex microbead assays (FIGS. 28E-G; MFI indicates the level of detected vesicles), sets of beads with individual internal infrared dye concentrations were coated with the indicated antibodies, washed and blocked according to the manufacturer's instructions (Luminex Corp., Austin, Tex.). cMVs were incubated and unbound cMVs were removed by washing. A second set of FITC labeled detector antibodies (anti-CD9, anti-CD63 and anti-CD81) were added for samples described in FIG. 28E and FIG. 28G. FIG. 28E shows a standard curve generated using the indicated amount of cMVs from the BrCa cell line MCF7. For FIG. 28F, patient cMVs were captured with anti-PCSA and detected with FITC-conjugated anti-SPDEF antibodies. Sample groups are indicated along the X-axis.

FIGS. 29A-I illustrate flow cytometric analysis of cancer-derived microvesicles in plasma from prostate cancer patients. FIG. 29A illustrates distribution of the patient cohort used in this study. FIGS. 29B-D illustrate biomarker frequencies on microvesicles from different patients. Microvesicles from plasma were processed and stained with PE conjugated primary antibodies (1 μg/well) and assessed by flow cytometry. Frequencies of PCSA+ events are plotted in FIG. 29B. Muc2 antigen expression was determined in the same cohort with PE-Cy7 conjugated aMuc2 Ab (FIG. 29C). Antigen expression of Adam10 detected by atto425 conjugated anti-Adam10 on the same microvesicles is shown in FIG. 29D. Distribution of the cohort in study is shown in (D). In each plot, the average and ±SEM in each condition are indicated. FIGS. 29E-H illustrate co-expression of the biomarkers and their frequencies on microvesicles from different patients. Microvesicles from plasma were processed and stained according with primary antibodies PE-labeled anti-PCSA and PE-Cy7-labeled anti-Muc2 (1 μg each per well) and acquired by flow cytometry. Ratio from SSC^{HI} EpCAM⁺vs SSC^{LO} EpCAM⁺ from double positive staining events were plotted in FIG. 29E. Muc2 and Adam10 antigen co-expression was analyzed in the same cohort and plotted in FIG. 29F. PCSA and Adam10 co-expression on the same cohort detected by PE-labeled anti-PCSA and Atto425-labeled anti-Adam10 cocktail is shown in FIG. 29G. Frequency of simultaneous expression of PCSA/Muc2/EpCAM/Adam10 on microvesicles is shown in FIG. 2911. Average and ±SEM in each condition is indicated in each plot. FIG. 29I illustrates quantification of EpCAM⁺SSC^{HI}/EpCAM⁺SSC^{LO} subpopulations of microvesicles from cancer and non-cancer plasma samples. Cohort samples were stained with antibodies to PCSA/EpCAM/Muc2/Adam10 and analyzed based on EpCAM expression on subpopulation with high and low SSC. Frequencies of SSC^{HI} with positive expression for EpCAM-Muc2-PCSA and Adam10 were compared with low SSC subpopulations in each sample and ratio normalized with normal samples.

FIGS. 30A-0 illustrate elements of the RISC complex within microvesicles and human plasma. FIGS. 30A-F illustrate levels of microRNAs let7a (FIG. 30A, FIG. 30C, FIG. 30E) and miR16 (FIG. 30B, FIG. 30D, FIG. 30F) detected under varying conditions from microvesicles from prostate cancer cell lines VCap (FIG. 30A, FIG. 30B), LNCap (FIG. 30C, FIG. 30D), and 22Rv1 (FIG. 30E, FIG. 30F). Immunoprecipitation (IP) was performed with antibodies to Ago2, CD81, BrdU (control), and mouse IgG (control). Amount of microvesicles was determined that co-immunoprecipitated with the various proteins. Amount of microRNAs that co-immunoprecipitated is shown on the Y axis and the protein target of the IP is shown on the X axis. The input sample comprised either whole microvesicles (“exosome”) or microvesicle lysate (“lysate”) as indicated in the legend. FIGS. 30G-H illustrate levels of microRNAs miR-16 (FIG. 30G) and miR-92a (FIG. 3011) detected in complex with Ago2 in human plasma. Immunoprecipitation (IP) was performed with antibodies to Ago2 and mouse IgG (control), as indicated in the figure legends. Amount of microRNAs that co-immunoprecipitated is shown on the Y axis and input volume is shown on the X axis. FIG. 30I shows Western blot analysis for Ago2 in Du145 lysate and purified VCaP microvesicles. FIG. 30J shows Western blot analysis for Ago2. GW182 was immunoprecipitated from human plasma followed by detection of Ago2 that co-immunoprecipitated with GW182. FIGS. 30K-L illustrate levels of microRNAs miR-92a (FIG. 30K) and miR-16 (FIG. 30L) detected in complex with GW182 and Ago2 in human plasma Immunoprecipitation (IP) was performed with antibodies to Ago2, GW182 and mouse IgG (control), as indicated in the figure legends. Amount of microRNAs that co-immunoprecipitated is shown on the Y axis and the protein target of the IP is shown on the X axis. The amounts of RNA were normalized to the anti-IgG control. FIGS. 30M-N illustrate levels of GW182:Ago2 complexes in various human plasma samples. Plate based ELISA was performed using anti-GW182 antibody as a capture agent and anti-Ago2 as a detection agent. FIG. 30M shows titration of sample input using purified microvesicles from cell line DU145, concentrated microvesicles from a plasma sample (“CN”), microvesicles from a plasma sample (“Neat”), and a no-sample control (“NS”). FIG. 30N shows levels of GW182:Ago2 detected in seven plasma samples. FIG. 30O shows levels of GW182:Ago2 complexes in various human urine samples. Microbead based ELISA was performed using anti-GW182 antibody or anti-Ago2 antibody as a capture agent and anti-Pan Argonaute as a detection agent. Conditions included raw urine vs cell positive hard spun urine (“+spin”). Amount of detected protein is shown on the Y axis and the protein target of the IP is shown on the X axis.

FIGS. 31A-F illustrate detection of microvesicles using lipid dyes and anti-protein antibodies. FIG. 31A and FIG. 31B illustrate staining of VCap derived vesicles. The vesicles were concentrated using ultrafiltration then stained simultaneously with an anti-tetraspanin-FITC cocktail (consisting antibodies to CD9, CD63, CD81), anti-EGFR-PE-Cy7 and the lipid dye DiI for 20 minutes at 37° C. while shaking. The solution was diluted with 500 μl PBS-BN, vortexed and analyzed on a MoFlo flow cytometer (Beckman Coulter, Inc., Indianapolis Ind.). In FIG. 31A, the vesicles were first gated for DiI+ events then EGFR+/tetraspanin+ events were counted. As indicated, 0% double negative events corresponding to cellular debris were observed. In FIG. 31B, the vesicles were first gated for tetraspanin+ events then EGFR+/DiI+ events were counted. As indicated, 29% double negative events corresponding to cellular debris were observed. FIG. 31C and FIG. 31D illustrate staining of vesicles concentrated from plasma of cancer-positive patients. Experimental conditions were otherwise identical to FIG. 31A and FIG. 31B, respectively. FIG. 31E and FIG. 31F illustrate staining of vesicles concentrated from plasma of cancer-negative patients. Experimental conditions were otherwise identical to FIG. 31A and FIG. 31B, respectively.

FIGS. 32A-E illustrate analysis of carboxyfluorescein diacetate succinimidyl ester (CFSE) stained microvesicles. Vesicles were isolated from human plasma samples using a procedure comprising thromboplastin-D treatment and ExoQuick isolation. Vesicles are incubated with non-fluorescent carboxyfluorescein diacetate succinimidyl ester (CFDA), which is converted to fluorescent CFSE by microvesicle esterases. See Examples for details. FIG. 32A shows serial dilution of vesicles stained with 40 μM of CFSE according to vendor instructions. After staining, the vesicles were serially diluted 11 times (see X axis) and fluorescence was detected coming from the conversion of non-fluorescent dye to its fluorescent ester form after microvesicle esterases remove the acetate groups (see Y axis). CFSE fluorescence was determined at several time-points (0, 15, 30 and 45 min post incubation, as indicated in the figure) to evaluate enzymatic activity over time. The CFSE fluorescent signal was consistent after 15 min of incubation and fluorescence values correleated to microvesicle concentration. Readings from negative control (sample without CFSE) or positive control (CFSE without microvesicles) were very low, indicating that autofluorescence or inactive CFSE does not significantly contribute to the detected fluorescence signal (data not shown). FIG. 32B shows a standard curve generated using CFSE stained microvesicles. 50×10⁶ microvesicles as determined using flow cytometry were stained with 40 μM in 400 μl to create the standard curve. The curve was generated by detecting fluorescence in a series of dilutions using a Viaa7 RT-PCR machine. See Examples for details. FIG. 32C shows the effects of CFSE concentration (μM) on microvesicle staining. The signal plateaued at ˜480 μM, indicating that the test samples and standard curve stained closer to 480 μM should minimize staining variation and signal will be due to cMV concentration. FIG. 32D and FIG. 32E illustrate determination of microvesicle concentration in a test sample using a standard curve. The protocol is outlined in detail in the Examples herein. Briefly, the standard curve and test samples were stained with 370 μM CFSE then incubated at room temperature before they were loaded on 96-well (MicroAmp) plate. In FIG. 32D, fluorescence relative units (Y-axis, Viia-7 system readings) were plotted against microvesicle concentration (X-axis). Linear regression was used to calculate a standard curve as shown in the plot. Based on the regression, two test samples of known concentration as determined by flow cytometry were stained with 370 μM CFSE and fluorescence was determined using the ViiA-7 system. Fluorescence values were interpolated to the standard curve to determine microvesicle concentration in the test samples. As seen in the table in FIG. 32E, determination of the concentration of microvesicles stained with CFSE dye agreed well with the flow cytometry data. Similar results were obtained using 480 μM CFSE to stain the microvesicles. When test samples were analyzed in triplicate, intersample CV % was lower when the sample was first stained and then aliquoted (CV=2.4%) versus when the sample was first aliquoted then stained (CV=15.33%). However, both methods yielded acceptable results.

FIGS. 33A and 33B illustrate a trivalent aptamer and use thereof. FIG. 33A illustrates an aptamer 330 consising of three regions: 1) a region 331 that binds a target molecule (i.e., antigen 1 or “Ag1”); 2) a linker region 332; and 3) a region 333 that binds a immunomodulatory target molecule (i.e., antigen 2 or “Ag2”). FIG. 33B illustrates recognition of aptamer 330 to a vesicle or cell 334. In the illustration, the aptamer 330 binds to two different antigens on the surface of the vesicle or cell 334. Region 331 of aptamer 330 binds to antigen 1 (Ag1) 335 and region 333 of aptamer 330 binds to antigen 2 (Ag2) 336.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

Disclosed herein are methods and systems for characterizing a phenotype of a biological sample, e.g., a sample from a cell culture, an organism, or a subject. The phenotype can be characterized by assessing one or more biomarkers. The biomarkers can be associated with a vesicle or vesicle population, either presented vesicle surface antigens or vesicle payload. As used herein, vesicle payload comprises entities encapsulated within a vesicle. Vesicle associated biomarkers can comprise both membrane bound and soluble biomarkers. The biomarkers can also be circulating biomarkers, such as nucleic acids (e.g., microRNA) or protein/polypeptide, or functional fragments thereof, assessed in a bodily fluid. Unless otherwise specified, the terms “purified” or “isolated” as used herein in reference to vesicles or biomarker components mean partial or complete purification or isolation of such components from a cell or organism. Furthermore, unless otherwise specified, reference to vesicle isolation using a binding agent includes binding a vesicle with the binding agent whether or not such binding results in complete isolation of the vesicle apart from other biological entities in the starting material.

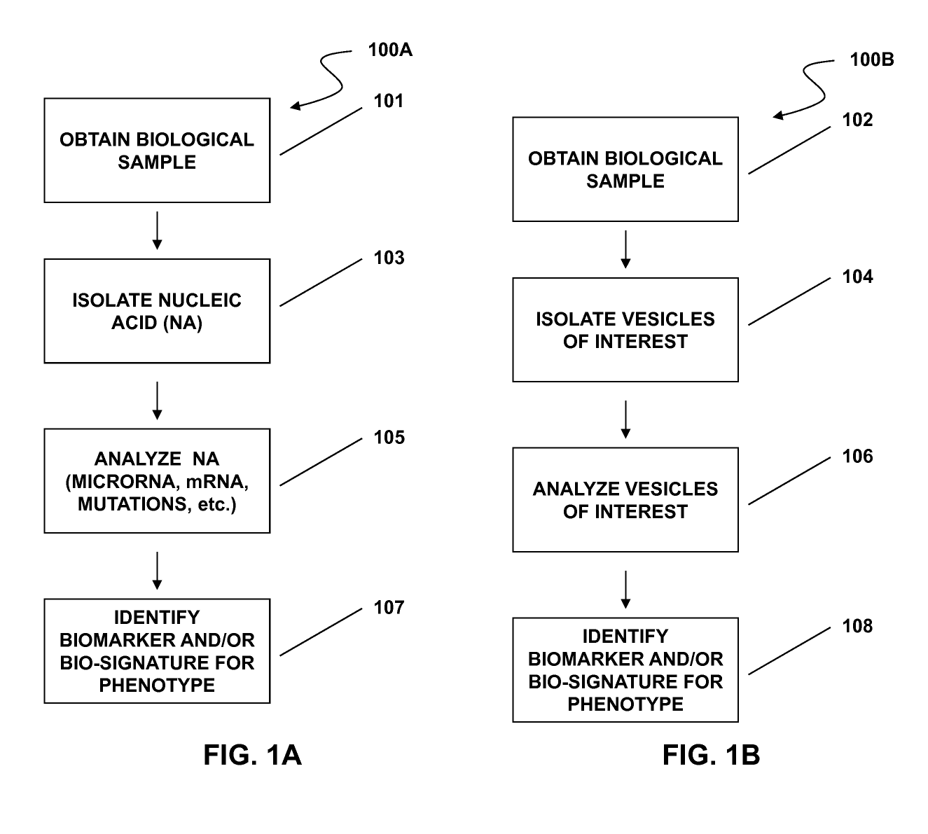

A method of characterizing a phenotype by analyzing a circulating biomarker, e.g., a nucleic acid biomarker, is depicted in scheme 100A of FIG. 1A, as a non-limiting illustrative example. In a first step 101, a biological sample is obtained, e.g., a bodily fluid, tissue sample or cell culture. Nucleic acids are isolated from the sample 103. The nucleic acid can be DNA or RNA, e.g., microRNA. Assessment of such nucleic acids can provide a biosignature for a phenotype. By sampling the nucleic acids associated with target phenotype (e.g., disease versus healthy, pre- and post-treatment), one or more nucleic acid markers that are indicative of the phenotype can be determined. Various aspects of the present invention are directed to biosignatures determined by assessing one or more nucleic acid molecules (e.g., microRNA) present in the sample 105, where the biosignature corresponds to a predetermined phenotype 107. FIG. 1B illustrates a scheme 100B of using vesicles to determine a biosignature and/or characterize a phenotype. In one example, a biological sample is obtained 102, and one or more vesicles of interest, e.g., all vesicles, or vesicles from a particular cell-of-origin and/or vesicles associated with a particular disease state, are isolated from the sample 104. The vesicles can be analyzed 106 by characterizing surface antigens associated with the vesicles and/or determining the presence or levels of components present within the vesicles (“payload”). Unless specified otherwise, the term “antigen” as used herein refers generally to a biomarker that can be bound by a binding agent, whether the binding agent is an antibody, aptamer, lectin, or other binding agent for the biomarker and regardless of whether such biomarker illicits an immune response in a host. Vesicle payload including without limitation protein, including peptides and polypeptides, nucleic acids such as DNA and RNAs, lipids and/or carbohydrates. RNA payload includes messenger RNA (mRNA) and microRNA (also referred to herein as miRNA or miR). A phenotype is characterized based on the biosignature of the vesicles 108. In another illustrative method of the invention, schemes 100A and 100B are performed together to characterize a phenotype. In such a scheme, vesicles and nucleic acids, e.g., microRNA, are assessed, thereby characterizing the phenotype.

According to the methods of the invention, multiple biomarkers can be assessed sequentially or concurrently to characterize a phenotype. For example, a subpopulation of vesicles can be assessed by concurrently detecting two vesicle surface antigens, e.g., using binding agents to both capture and detect vesicles. In another example, a subpopulation of vesicles can be assessed by sequentially detecting a vesicle surface antigen, e.g., to capture vesicles, and then the captured vesicles can be assessed for payload such as mRNA, microRNA or soluble protein. In some embodiments, characterizing a phenotype comprises both the concurrent assessment of one or more biomarker and sequential assessment of one or more other biomarker. As a non-limiting example, a vesicle subpopulation that is detecting using binding agents to more than one surface antigen can be sorted, and then payload can be assessed, e.g., one or more miRs. One of skill will recognize that many variations of sequential or concurrent assessment of biomarkers can be used to characterize a phenotype.

In another related aspect, methods are provided herein for the discovery of biomarkers comprising assessing vesicle surface markers or payload markers in one sample and comparing the markers to another sample. Markers that distinguish between the samples can be used as biomarkers according to the invention. Such samples can be from a subject or group of subjects. For example, the groups can be, e.g., diseased versus normal (e.g., non-diseased), known responders and non-responders to a given treatment for a given disease or disorder. Biomarkers discovered to distinguish the known responders and non-responders provide a biosignature of whether a subject is likely to respond to a treatment such as a therapeutic agent, e.g., a drug or biologic.

To address the problem of immunosuppression resulting from a cancer, the invention further provides compositions and methods for inhibiting immunosuppressive factors produced by cancer cells both at their source and when secreted as microvesicles. Antibody therapies have been tested in animal models and early human trials with limited success. Often the host develops anti-idiotypic antibodies rendering such therapies ineffective. In addition, there can be many immunosuppressive factors related to cancer so blocking a single factor may not be sufficient to re-introduce an effective host immune response against the cancer. Thus, immunosuppressive pathways may compensate for the blocked immunosuppressive factor by such antibodies. The invention can address such multiple tumor-associated immunosuppressive factors secreted by the tumor.

The invention further provides compositions and methods for inhibiting immunosuppressive factor as well as stimulating the interacting host immune cells.

In an aspect, the invention provides therapeutic agents that bind to tumor-derived circulating microvesicles (cMVs). The therapeutic agents can inhibit an immunosuppressive factor on the cMVs and also stimulate the interacting immune cell to resist other immunosuppressive factors and support or induce anti-tumor immunity. Because cMVs may resemble their cell of origin regarding membrane structure, the therapeutic agent may further provide synergistic impact by inhibiting such immunosuppressive factors on the cancer cells themselves.

In an embodiment, the therapeutic agent of the invention comprises a nucleic acid oligonucleotide, such as an aptamer. In an embodiment, the oligonucleotide comprises DNA. The oligonucleotide can be synthetic. Aptamers for a given target are created by randomly generating oligonucleotides of a specific length, typically 20-40 base pairs long, e.g., 20, 21, 22, 23, 24, 25, 26, 27, 28, 29, 30, 31, 32, 33, 34, 35, 36, 37, 38, or 40 base pairs. These random oligonucleotides are then incubated with the protein target of interest. After several wash steps, the oligonucleotides that bind to the target are collected and amplified. The amplified aptamers are then added to the target and the process is repeated, often 15-20 times. A common version of this process known to those of skill in the art as the SELEX method, which is described further herein. The end result comprises one or more aptamer with high affinity to the target. The aptamers of the invention can comprise multiple such target binding sites separated by a linker.

Following long-standing patent law convention, the terms “a”, “an”, and “the” refer to “one or more” when used in this application, including the claims. Thus, for example, reference to “a biomarker” includes a plurality of such biomarkers, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter. As used herein, the term “about,” e.g., when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. In embodiments, “about” refers to ±10%.

### Phenotypes

Disclosed herein are products and processes for characterizing a phenotype using the methods and compositions of the invention. The term “phenotype” as used herein can mean any trait or characteristic that is attributed to a biomarker profile that is identified using in part or in whole the compositions and/or methods of the invention. For example, a phenotype can be a diagnostic, prognostic or theranostic determination based on a characterized biomarker profile for a sample obtained from a subject. A phenotype can be any observable characteristic or trait of, such as a disease or condition, a stage of a disease or condition, susceptibility to a disease or condition, prognosis of a disease stage or condition, a physiological state, or response/potential response to therapeutics. A phenotype can result from a subject's genetic makeup as well as the influence of environmental factors and the interactions between the two, as well as from epigenetic modifications to nucleic acid sequences.

A phenotype in a subject can be characterized by obtaining a biological sample from a subject and analyzing the sample. For example, characterizing a phenotype for a subject or individual may include detecting a disease or condition (including pre-symptomatic early stage detecting), determining a prognosis, diagnosis, or theranosis of a disease or condition, or determining the stage or progression of a disease or condition. Characterizing a phenotype can include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation. The products and processes described herein allow assessment of a subject on an individual basis, which can provide benefits of more efficient and economical decisions in treatment.

In an aspect, the invention relates to the analysis of a biological sample to identify a biosignature to predict whether a subject is likely to respond to a treatment for a disease or disorder. Characterizating a phenotype includes predicting the responder/non-responder status of the subject, wherein a responder responds to a treatment for a disease and a non-responder does not respond to the treatment. Vesicles can be analyzed in the subject and compared to vesicle analysis of previous subjects that were known to respond or not to a treatment. If the vesicle biosignature in a subject more closely aligns with that of previous subjects that were known to respond to the treatment, the subject can be characterized, or predicted, as a responder to the treatment. Similarly, if the vesicle biosignature in the subject more closely aligns with that of previous subjects that did not respond to the treatment, the subject can be characterized, or predicted as a non-responder to the treatment. The treatment can be for any appropriate disease, disorder or other condition. The method can be used in any disease setting where a vesicle biosignature that correlates with responder/non-responder status is known.

In some embodiments, the phenotype comprises a disease or condition such as those listed in Table 1. For example, the phenotype can comprise the presence of or likelihood of developing a tumor, neoplasm, or cancer. A cancer detected or assessed by products or processes described herein includes, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma.

The phenotype can be a premalignant condition, such as actinic keratosis, atrophic gastritis, leukoplakia, erythroplasia, Lymphomatoid Granulomatosis, preleukemia, fibrosis, cervical dysplasia, uterine cervical dysplasia, xeroderma pigmentosum, Barrett's Esophagus, colorectal polyp, or other abnormal tissue growth or lesion that is likely to develop into a malignant tumor. Transformative viral infections such as HIV and HPV also present phenotypes that can be assessed according to the invention.

A cancer characterized by the methods of the invention can comprise, without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposis sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer under analysis may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The methods of the invention can be used to characterize these and other cancers. Thus, characterizing a phenotype can be providing a diagnosis, prognosis or theranosis of one of the cancers disclosed herein.

In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The methods of the invention can be used to characterize these and other cancers. Thus, characterizing a phenotype can be providing a diagnosis, prognosis or theranosis of one of the cancers disclosed herein.

The phenotype can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

The phenotype can also comprise a cardiovascular disease, such as atherosclerosis, congestive heart failure, vulnerable plaque, stroke, or ischemia. The cardiovascular disease or condition can be high blood pressure, stenosis, vessel occlusion or a thrombotic event.

The phenotype can also comprise a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain.

The phenotype may also comprise an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. Viral proteins, such as HIV or HCV-like particles can be assessed in a vesicle, to characterize a viral condition.

The phenotype can also comprise a perinatal or pregnancy related condition (e.g. preeclampsia or preterm birth), metabolic disease or condition, such as a metabolic disease or condition associated with iron metabolism. For example, hepcidin can be assayed in a vesicle to characterize an iron deficiency. The metabolic disease or condition can also be diabetes, inflammation, or a perinatal condition.

The methods of the invention can be used to characterize these and other diseases and disorders that can be assessed via a candidate biosignature comprising one or a plurality of biomarkers. Thus, characterizing a phenotype can be providing a diagnosis, prognosis or theranosis of one of the diseases and disorders disclosed herein.

In various embodiments of the invention, a biosignature for any of the conditions or diseases disclosed herein can comprise one or more biomarkers in one of several different categories of markers, wherein the categories include one or more of: 1) disease specific biomarkers; 2) cell- or tissue-specific biomarkers; 3) vesicle-specific markers (e.g., general vesicle biomarkers); 4. angiogenesis-specific biomarkers; and 5) immunomodulatory biomarkers. Examples of such markers for use in methods and compositions of the invention are disclosed herein. Furthermore, a biomarker known in the art that is characterized to have a role in a particular disease or condition can be adapted for use as a target in compositions and methods of the invention. In further embodiments, such biomarkers can be all vesicle surface markers, or a combination of vesicle surface markers and vesicle payload markers (i.e., molecules enclosed by a vesicle). In addition, as noted herein, the biological sample assessed can be any biological fluid, or can comprise individual components present within such biological fluid (e.g., vesicles, nucleic acids, proteins, or complexes thereof).

### Subject

One or more phenotypes of a subject can be determined by analyzing one or more vesicles, such as vesicles, in a biological sample obtained from the subject. A subject or patient can include, but is not limited to, mammals such as bovine, avian, canine, equine, feline, ovine, porcine, or primate animals (including humans and non-human primates). A subject can also include a mammal of importance due to being endangered, such as a Siberian tiger; or economic importance, such as an animal raised on a farm for consumption by humans, or an animal of social importance to humans, such as an animal kept as a pet or in a zoo. Examples of such animals include, but are not limited to, carnivores such as cats and dogs; swine including pigs, hogs and wild boars; ruminants or ungulates such as cattle, oxen, sheep, giraffes, deer, goats, bison, camels or horses. Also included are birds that are endangered or kept in zoos, as well as fowl and more particularly domesticated fowl, i.e. poultry, such as turkeys and chickens, ducks, geese, guinea fowl. Also included are domesticated swine and horses (including race horses). In addition, any animal species connected to commercial activities are also included such as those animals connected to agriculture and aquaculture and other activities in which disease monitoring, diagnosis, and therapy selection are routine practice in husbandry for economic productivity and/or safety of the food chain.

The subject can have a pre-existing disease or condition, such as cancer. Alternatively, the subject may not have any known pre-existing condition. The subject may also be non-responsive to an existing or past treatment, such as a treatment for cancer.

### Samples

A sample used and/or assessed via the compositions and methods of the invention includes any relevant biological sample that can be used for biomarker assessment, including without limitation sections of tissues such as biopsy or tissue removed during surgical or other procedures, bodily fluids, autopsy samples, frozen sections taken for histological purposes, and cell cultures. Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+formalin fixative. More than one sample of more than one type can be used for each patient.

The sample used in the methods described herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In an embodiment, the fixed tissue comprises a tumor containing formalin fixed paraffin embedded (FFPE) block from a surgery or biopsy. In another embodiment, the unstained slides comprise unstained, charged, unbaked slides from a paraffin block. In another embodiment, bone marrow core or clot comprises a decalcified core. A formalin fixed core and/or clot can be paraffin-embedded. In still another embodiment, the core needle biopsy comprises 1, 2, 3, 4, 5, 6, 7, 8, 9, 10 or more, e.g., 3-4, paraffin embedded biopsy samples. An 18 gauge needle biopsy can be used. The malignant fluid can comprise a sufficient volume of fresh pleural/ascitic fluid to produce a 5×5×2 mm cell pellet. The fluid can be formalin fixed in a paraffin block. In an embodiment, the core needle biopsy comprises 1, 2, 3, 4, 5, 6, 7, 8, 9, 10 or more, e.g., 4-6, paraffin embedded aspirates.

A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif. (1990).

The biological sample assessed using the compositions and methods of the invention can be any useful bodily or biological fluid, including but not limited to peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, other lavage fluids, cells, cell culture, or a cell culture supernatant. A biological sample may also include the blastocyl cavity, umbilical cord blood, or maternal circulation which may be of fetal or maternal origin. The biological sample may also be a cell culture, tissue sample or biopsy from which vesicles and other circulating biomarkers may be obtained. For example, cells of interest can be cultured and vesicles isolated from the culture. In various embodiments, biomarkers or more particularly biosignatures disclosed herein can be assessed directly from such biological samples (e.g., identification of presence or levels of nucleic acid or polypeptide biomarkers or functional fragments thereof) using various methods, such as extraction of nucleic acid molecules from blood, plasma, serum or any of the foregoing biological samples, use of protein or antibody arrays to identify polypeptide (or functional fragment) biomarker(s), as well as other array, sequencing, PCR and proteomic techniques known in the art for identification and assessment of nucleic acid and polypeptide molecules. In addition, one or more components present in such samples can be first isolated or enriched and further processed to assess the presence or levels of selected biomarkers, to assess a given biosignature (e.g., isolated microvesicles prior to profiling for protein and/or nucleic acid biomarkers).

Table 1 presents a non-limiting listing of diseases, conditions, or biological states and corresponding biological samples that may be used for analysis according to the methods of the invention.

TABLE 1
Examples of Biological Samples for Vesicle Analysis for
Various Diseases, Conditions, or Biological States

| Illustrative Disease, Condition or Biological State | Illustrative Biological Samples |
|---|---|
| Cancers/neoplasms affecting the following tissue | Tumor sample, blood, serum, plasma, cerebrospinal |
| types/bodily systems: breast, lung, ovarian, colon, | fluid (CSF), urine, sputum, ascites, synovial fluid, |
| rectal, prostate, pancreatic, brain, bone, connective | semen, nipple aspirates, saliva, bronchoalveolar lavage |
| tissue, glands, skin, lymph, nervous system, endocrine, | fluid, tears, oropharyngeal washes, feces, peritoneal |
| germ cell, genitourinary, hematologic/blood, bone | fluids, pleural effusion, sweat, tears, aqueous humor, |
| marrow, muscle, eye, esophageal, fat tissue, thyroid, | pericardial fluid, lymph, chyme, chyle, bile, stool |
| pituitary, spinal cord, bile duct, heart, gall bladder, | water, amniotic fluid, breast milk, pancreatic juice, |
| bladder, testes, cervical, endometrial, renal, ovarian, | cerumen, Cowper's fluid or pre-ejaculatory fluid, |
| digestive/gastrointestinal, stomach, head and neck, | female ejaculate, interstitial fluid, menses, mucus, pus, |
| liver, leukemia, respiratory/thorasic, cancers of | sebum, vaginal lubrication, vomit |
| unknown primary (CUP) | |
| Neurodegenerative/neurological disorders: | Blood, serum, plasma, CSF, urine |
| Parkinson's disease, Alzheimer's Disease and multiple | |
| sclerosis, Schizophrenia, and bipolar disorder, | |
| spasticity disorders, epilepsy | |
| Cardiovascular Disease: atherosclerosis, | Blood, serum, plasma, CSF, urine |
| cardiomyopathy, endocarditis, vunerable plaques, | |
| infection | |
| Stroke: ischemic, intracerebral hemorrhage, | Blood, serum, plasma, CSF, urine |
| subarachnoid hemorrhage, transient ischemic attacks | |
| (TIA) | |
| Pain disorders: peripheral neuropathic pain and | Blood, serum, plasma, CSF, urine |
| chronic neuropathic pain, and fibromyalgia, | |
| Autoimmune disease: systemic and localized diseases, | Blood, serum, plasma, CSF, urine, synovial fluid |
| rheumatic disease, Lupus, Sjogren's syndrome | |
| Digestive system abnormalities: Barrett's esophagus, | Blood, serum, plasma, CSF, urine |
| irritable bowel syndrome, ulcerative colitis, Crohn's | |
| disease, Diverticulosis and Diverticulitis, Celiac | |
| Disease | |
| Endocrine disorders: diabetes mellitus, various forms | Blood, serum, plasma, CSF, urine |
| of Thyroiditis, adrenal disorders, pituitary disorders | |
| Diseases and disorders of the skin: psoriasis | Blood, serum, plasma, CSF, urine, synovial fluid, tears |
| Urological disorders: benign prostatic hypertrophy | Blood, serum, plasma, urine |
| (BPH), polycystic kidney disease, interstitial cystitis | |
| Hepatic disease/injury: Cirrhosis, induced | Blood, serum, plasma, urine |
| hepatotoxicity (due to exposure to natural or synthetic | |
| chemical sources) | |
| Kidney disease/injury: acute, sub-acute, chronic | Blood, serum, plasma, urine |
| conditions, Podocyte injury, focal segmental | |
| glomerulosclerosis | |
| Endometriosis | Blood, serum, plasma, urine, vaginal fluids |
| Osteoporosis | Blood, serum, plasma, urine, synovial fluid |
| Pancreatitis | Blood, serum, plasma, urine, pancreatic juice |
| Asthma | Blood, serum, plasma, urine, sputum, bronchiolar |
| | lavage fluid |
| Allergies | Blood, serum, plasma, urine, sputum, bronchiolar |
| | lavage fluid |
| Prion-related diseases | Blood, serum, plasma, CSF, urine |
| Viral Infections: HIV/AIDS | Blood, serum, plasma, urine |
| Sepsis | Blood, serum, plasma, urine, tears, nasal lavage |
| Organ rejection/transplantation | Blood, serum, plasma, urine, various lavage fluids |
| Differentiating conditions: adenoma versus | Blood, serum, plasma, urine, sputum, feces, colonic |
| hyperplastic polyp, irritable bowel syndrome (IBS) | lavage fluid |
| versus normal, classifying Dukes stages A, B, C, | |
| and/or D of colon cancer, adenoma with low-grade | |
| hyperplasia versus high-grade hyperplasia, adenoma | |
| versus normal, colorectal cancer versus normal, IBS | |
| versus. ulcerative colitis (UC) versus Crohn's disease | |
| (CD), | |
| Pregnancy related physiological states, conditions, or | Maternal serum, plasma, amniotic fluid, cord blood |
| affiliated diseases: genetic risk, adverse pregnancy | |
| outcomes | |

The methods of the invention can be used to characterize a phenotype using a blood sample or blood derivative. Blood derivatives include plasma and serum. Blood plasma is the liquid component of whole blood, and makes up approximately 55% of the total blood volume. It is composed primarily of water with small amounts of minerals, salts, ions, nutrients, and proteins in solution. In whole blood, red blood cells, leukocytes, and platelets are suspended within the plasma. Blood serum refers to blood plasma without fibrinogen or other clotting factors (i.e., whole blood minus both the cells and the clotting factors).

The biological sample may be obtained through a third party, such as a party not performing the analysis of the biomarkers, whether direct assessment of a biological sample or by profiling one or more vesicles obtained from the biological sample. For example, the sample may be obtained through a clinician, physician, or other health care manager of a subject from which the sample is derived. Alternatively, the biological sample may obtained by the same party analyzing the vesicle. In addition, biological samples be assayed, are archived (e.g., frozen) or otherwise stored in under preservative conditions.

The volume of the biological sample used for biomarker analysis can be in the range of between 0.1-20 mL, such as less than about 20, 15, 10, 9, 8, 7, 6, 5, 4, 3, 2, 1 or 0.1 mL.

A sample of bodily fluid can be used as a sample for characterizing a phenotype. For example, biomarkers in the sample can be assessed to provide a diagnosis, prognosis and/or theranosis of a disease. The biomarkers can be circulating biomarkers, such as circulating proteins or nucleic acids. The biomarkers can also be associated with a vesicle or vesicle population. Methods of the invention can be applied to assess one or more vesicles, as well as one or more different vesicle populations that may be present in a biological sample or in a subject. Analysis of one or more biomarkers in a biological sample can be used to determine whether an additional biological sample should be obtained for analysis. For example, analysis of one or more vesicles in a sample of bodily fluid can aid in determining whether a tissue biopsy should be obtained.

A sample from a patient can be collected under conditions that preserve the circulating biomarkers and other entities of interest contained therein for subsequent analysis. In an embodiment, the samples are processed using one or more of CellSave Preservative Tubes (Veridex, North Raritan, N.J.), PAXgene Blood DNA Tubes (QIAGEN GmbH, Germany), and RNAlater (QIAGEN GmbH, Germany).

CellSave Preservative Tubes (CellSave tubes) are sterile evacuated blood collection tubes. Each tube contains a solution that contains Na2EDTA and a cell preservative. The EDTA absorbs calcium ions, which can reduce or eliminate blood clotting. The preservative preserves the morphology and cell surface antigen expression of epithelial and other cells. The collection and processing can be performed as described in a protocol provided by the manufacturer. Each tube is evacuated to withdraw venous whole blood following standard phlebotomy procedures as known to those of skill in the art. CellSave tubes are disclosed in U.S. Pat. Nos. 5,466,574; 5,512,332; 5,597,531; 5,698,271; 5,985,153; 5,993,665; 6,120,856; 6,136,182; 6,365,362; 6,551,843; 6,620,627; 6,623,982; 6,645,731; 6,660,159; 6,790,366; 6,861,259; 6,890,426; 7,011,794; 7,282,350; 7,332,288; 5,849,517 and 5,459,073, each of which is incorporated by reference in its entirety herein.

The PAXgene Blood DNA Tube (PAXgene tube) is a plastic, evacuated tube for the collection of whole blood for the isolation of nucleic acids. The tubes can be used for blood collection, transport and storage of whole blood specimens and isolation of nucleic acids contained therein, e.g., DNA or RNA. Blood is collected under a standard phlebotomy protocol into an evacuated tube that contains an additive. The collection and processing can be performed as described in a protocol provided by the manufacturer. PAXgene tubes are disclosed in U.S. Pat. Nos. 5,906,744; 4,741,446; 4,991,104, each of which is incorporated by reference in its entirety herein.

The RNAlater RNA Stabilization Reagent (RNAlater) is used for immediate stabilization of RNA in tissues. RNA can be unstable in harvested samples. The aqueous RNAlater reagent permeates tissues and other biological samples, thereby stabilizing and protecting the RNA contained therein. Such protection helps ensure that downstream analyses reflect the expression profile of the RNA in the tissue or other sample. The samples are submerged in an appropriate volume of RNAlater reagent immediately after harvesting. The collection and processing can be performed as described in a protocol provided by the manufacturer. According to the manufacturer, the reagent preserves RNA for up to 1 day at 37° C., 7 days at 18-25° C., or 4 weeks at 2-8° C., allowing processing, transportation, storage, and shipping of samples without liquid nitrogen or dry ice. The samples can also be placed at −20° C. or −80° C., e.g., for archival storage. The preserved samples can be used to analyze any type of RNA, including without limitation total RNA, mRNA, and microRNA. RNAlater can also be useful for collecting samples for DNA, RNA and protein analysis. RNAlater is disclosed in U.S. Pat. No. 5,346,994, each of which is incorporated by reference in its entirety herein.

Unless otherwise specified, the biological sample of the invention is understood to comprise a sample containing a separated, depleted, enriched, isolated, or otherwise processed derivative of another biological sample. As a non-limiting example, a component of a patient sample or a cell culture can be isolated from the patient sample or the cell culture and resuspended in a buffer for further analysis. One of skill will appreciate that the derivative component suspended in the buffer is a biological sample that can be assessed according to the methods of the invention. The component can be any useful biological entity as disclosed herein or known in the art, including without limitation circulating biomarkers, vesicles, proteins, nucleic acids, lipids or carbohydrates. The biological sample can be the biological entity, including without limitation circulating biomarkers, vesicles, proteins, nucleic acids, lipids or carbohydrates.

### Vesicles

Methods of the invention can include assessing one or more vesicles, including assessing vesicle populations. A vesicle, as used herein, is a membrane vesicle that is shed from cells. Vesicles or membrane vesicles include without limitation: circulating microvesicles (cMVs), microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phospholipid vesicle, liposomal vesicle, argosome, texasome, secresome, tolerosome, melanosome, oncosome, or exocytosed vehicle. Furthermore, although vesicles may be produced by different cellular processes, the methods of the invention are not limited to or reliant on any one mechanism, insofar as such vesicles are present in a biological sample and are capable of being characterized by the methods disclosed herein. Unless otherwise specified, methods that make use of a species of vesicle can be applied to other types of vesicles. Vesicles comprise spherical structures with a lipid bilayer similar to cell membranes which surrounds an inner compartment which can contain soluble components, sometimes referred to as the payload. In some embodiments, the methods of the invention make use of exosomes, which are small secreted vesicles of about 40-100 nm in diameter. For a review of membrane vesicles, including types and characterizations, see Thery et al., Nat Rev Immunol. 2009 Aug. 9(8):581-93. Some properties of different types of vesicles include those in Table 2:

TABLE 2
Vesicle Properties

| | | | | Membrane | Exosome- | Apoptotic |
|---|---|---|---|---|---|---|
| Feature | Exosomes | Microvesicles | Ectosomes | particles | like vesicles | vesicles |
| Size | 50-100 nm | 100-1,000 nm | 50-200 nm | 50-80 nm | 20-50 nm | 50-500 nm |
| Density in | 1.13-1.19 g/ml | | | 1.04-1.07 g/ml | 1.1 g/ml | 1.16-1.28 g/ml |
| sucrose | | | | | | |
| EM | Cup shape | Irregular | Bilamellar | Round | Irregular | Heterogeneous |
| appearance | | shape, | round | | shape | |
| | | electron | structures | | | |
| | | dense | | | | |
| Sedimentation | 100,000 g | 10,000 g | 160,000- | 100,000- | 175,000 g | 1,200 g, 10,000 |
| | | | 200,000 g | 200,000 g | | g, 100,000 g |
| Lipid | Enriched in | Expose PPS | Enriched in | | No lipid | |
| composition | cholesterol, | | cholesterol and | | rafts | |
| | sphingomyelin | | diacylglycerol; | | | |
| | and ceramide; | | expose PPS | | | |
| | contains lipid | | | | | |
| | rafts; expose | | | | | |
| | PPS | | | | | |
| Major protein | Tetraspanins | Integrins, | CR1 and | CD133; no | TNFRI | Histones |
| markers | (e.g., CD63, | selectins and | proteolytic | CD63 | | |
| | CD9), Alix, | CD40 ligand | enzymes; no | | | |
| | TSG101 | | CD63 | | | |
| Intracellular | Internal | Plasma | Plasma | Plasma | | |
| origin | compartments | membrane | membrane | membrane | | |
| | (endosomes) | | | | | |

Abbreviations:
phosphatidylserine (PPS);
electron microscopy (EM)

Vesicles include shed membrane bound particles, or “microparticles,” that are derived from either the plasma membrane or an internal membrane. Vesicles can be released into the extracellular environment from cells. Cells releasing vesicles include without limitation cells that originate from, or are derived from, the ectoderm, endoderm, or mesoderm. The cells may have undergone genetic, environmental, and/or any other variations or alterations. For example, the cell can be tumor cells. A vesicle can reflect any changes in the source cell, and thereby reflect changes in the originating cells, e.g., cells having various genetic mutations. In one mechanism, a vesicle is generated intracellularly when a segment of the cell membrane spontaneously invaginates and is ultimately exocytosed (see for example, Keller et al., Immunol. Lett. 107 (2): 102-8 (2006)). Vesicles also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). A vesicle shed into circulation or bodily fluids from tumor cells may be referred to as a “circulating tumor-derived vesicle.” When such vesicle is an exosome, it may be referred to as a circulating-tumor derived exosome (CTE). In some instances, a vesicle can be derived from a specific cell of origin. CTE, as with a cell-of-origin specific vesicle, typically have one or more unique biomarkers that permit isolation of the CTE or cell-of-origin specific vesicle, e.g., from a bodily fluid and sometimes in a specific manner. For example, a cell or tissue specific markers are used to identify the cell of origin. Examples of such cell or tissue specific markers are disclosed herein and can further be accessed in the Tissue-specific Gene Expression and Regulation (TiGER) Database, available at bioinfo.wilmer.jhu.edu/tiger/; Liu et al. (2008) TiGER: a database for tissue-specific gene expression and regulation. BMC Bioinformatics. 9:271; TissueDistributionDBs, available at genome dkfz-heidelberg.de/menu/tissue_db/index.html.

A vesicle can have a diameter of greater than about 10 nm, 20 nm, or 30 nm. A vesicle can have a diameter of greater than 40 nm, 50 nm, 100 nm, 200 nm, 500 nm, 1000 nm, 1500 nm, 2000 nm or greater than 10,000 nm. A vesicle can have a diameter of about 20-2000 nm, about 20-1500 nm, about 30-1000 nm, about 30-800 nm, about 30-200 nm, or about 30-100 nm. In some embodiments, the vesicle has a diameter of less than 10,000 nm, 2000 nm, 1500 nm, 1000 nm, 800 nm, 500 nm, 200 nm, 100 nm, 50 nm, 40 nm, 30 nm, 20 nm or less than 10 nm. As used herein the term “about” in reference to a numerical value means that variations of 10% above or below the numerical value are within the range ascribed to the specified value. Typical sizes for various types of vesicles are shown in Table 2. Vesicles can be assessed to measure the diameter of a single vesicle or any number of vesicles. For example, the range of diameters of a vesicle population or an average diameter of a vesicle population can be determined. Vesicle diameter can be assessed using methods known in the art, e.g., imaging technologies such as electron microscopy. In an embodiment, a diameter of one or more vesicles is determined using optical particle detection. See, e.g., U.S. Pat. No. 7,751,053, entitled “Optical Detection and Analysis of Particles” and issued Jul. 6, 2010; and U.S. Pat. No. 7,399,600, entitled “Optical Detection and Analysis of Particles” and issued Jul. 15, 2010.

In some embodiments, the methods of the invention comprise assessing vesicles directly from a biological sample without prior isolation, purification, or concentration from the biological sample. For example, the amount of vesicles in the sample can by itself provide a biosignature that provides a diagnostic, prognostic or theranostic determination. Alternatively, the vesicle in the sample may be isolated, captured, purified, or concentrated from a sample prior to analysis. As noted, isolation, capture or purification as used herein comprises partial isolation, partial capture or partial purification apart from other components in the sample. Vesicle isolation can be performed using various techniques as described herein, e.g., chromatography, filtration, centrifugation, flow cytometry, affinity capture (e.g., to a planar surface or bead), and/or using microfluidics.

Vesicles such as exosomes can be assessed to provide a phenotypic characterization by comparing vesicle characteristics to a reference. In some embodiments, surface antigens on a vesicle are assessed. The surface antigens can provide an indication of the anatomical origin and/or cellular of the vesicles and other phenotypic information, e.g., tumor status. For example, wherein vesicles found in a patient sample, e.g., a bodily fluid such as blood, serum or plasma, are assessed for surface antigens indicative of colorectal origin and the presence of cancer. The surface antigens may comprise any informative biological entity that can be detected on the vesicle membrane surface, including without limitation surface proteins, lipids, carbohydrates, and other membrane components. For example, positive detection of colon derived vesicles expressing tumor antigens can indicate that the patient has colorectal cancer. As such, methods of the invention can be used to characterize any disease or condition associated with an anatomical or cellular origin, by assessing, for example, disease-specific and cell-specific biomarkers of one or more vesicles obtained from a subject.

In another embodiment, the methods of the invention comprise assessing one or more vesicle payloads to provide a phenotypic characterization. The payload with a vesicle comprises any informative biological entity that can be detected as encapsulated within the vesicle, including without limitation proteins and nucleic acids, e.g., genomic or cDNA, mRNA, or functional fragments thereof, as well as microRNAs (miRs). In addition, methods of the invention are directed to detecting vesicle surface antigens (in addition or exclusive to vesicle payload) to provide a phenotypic characterization. For example, vesicles can be characterized by using binding agents (e.g., antibodies or aptamers) that are specific to vesicle surface antigens, and the bound vesicles can be further assessed to identify one or more payload components disclosed therein. As described herein, the levels of vesicles with surface antigens of interest or with payload of interest can be compared to a reference to characterize a phenotype. For example, overexpression in a sample of cancer-related surface antigens or vesicle payload, e.g., a tumor associated mRNA or microRNA, as compared to a reference, can indicate the presence of cancer in the sample. The biomarkers assessed can be present or absent, increased or reduced based on the selection of the desired target sample and comparison of the target sample to the desired reference sample. Non-limiting examples of target samples include: disease; treated/not-treated; different time points, such as a in a longitudinal study; and non-limiting examples of reference sample: non-disease; normal; different time points; and sensitive or resistant to candidate treatment(s).

### MicroRNA

Various biomarker molecules can be assessed in biological samples or vesicles obtained from such biological samples. MicroRNAs comprise one class biomarkers assessed via methods of the invention. MicroRNAs, also referred to herein as miRNAs or miRs, are short RNA strands approximately 21-23 nucleotides in length. MiRNAs are encoded by genes that are transcribed from DNA but are not translated into protein and thus comprise non-coding RNA. The miRs are processed from primary transcripts known as pri-miRNA to short stem-loop structures called pre-miRNA and finally to the resulting single strand miRNA. The pre-miRNA typically forms a structure that folds back on itself in self-complementary regions. These structures are then processed by the nuclease Dicer in animals or DCL1 in plants. Mature miRNA molecules are partially complementary to one or more messenger RNA (mRNA) molecules and can function to regulate translation of proteins. Identified sequences of miRNA can be accessed at publicly available databases, such as www.microRNA.org, www.mirbase.org, or www.mirz.unibas.ch/cgi/miRNA.cgi.

miRNAs are generally assigned a number according to the naming convention “mir-[number].” The number of a miRNA is assigned according to its order of discovery relative to previously identified miRNA species. For example, if the last published miRNA was mir-121, the next discovered miRNA will be named mir-122, etc. When a miRNA is discovered that is homologous to a known miRNA from a different organism, the name can be given an optional organism identifier, of the form [organism identifier]-mir-[number]. Identifiers include hsa for Homo sapiens and mmu for Mus Musculus. For example, a human homolog to mir-121 might be referred to as hsa-mir-121 whereas the mouse homolog can be referred to as mmu-mir-121 and the rat homolog can be referred to as rno-mir-121, etc.

Mature microRNA is commonly designated with the prefix “miR” whereas the gene or precursor miRNA is designated with the prefix “mir.” For example, mir-121 is a precursor for miR-121. When differing miRNA genes or precursors are processed into identical mature miRNAs, the genes/precursors can be delineated by a numbered suffix. For example, mir-121-1 and mir-121-2 can refer to distinct genes or precursors that are processed into miR-121. Lettered suffixes are used to indicate closely related mature sequences. For example, mir-121a and mir-121b can be processed to closely related miRNAs miR-121a and miR-121b, respectively. In the context of the invention, any microRNA (miRNA or miR) designated herein with the prefix mir-* or miR-* is understood to encompass both the precursor and/or mature species, unless otherwise explicitly stated otherwise.

Sometimes it is observed that two mature miRNA sequences originate from the same precursor. When one of the sequences is more abundant that the other, a “*” suffix can be used to designate the less common variant. For example, miR-121 would be the predominant product whereas miR-121* is the less common variant found on the opposite arm of the precursor. If the predominant variant is not identified, the miRs can be distinguished by the suffix “5p” for the variant from the 5′ arm of the precursor and the suffix “3p” for the variant from the 3′ arm. For example, miR-121-5p originates from the 5′ arm of the precursor whereas miR-121-3p originates from the 3′ arm. Less commonly, the 5p and 3p variants are referred to as the sense (“s”) and anti-sense (“as”) forms, respectively. For example, miR-121-5p may be referred to as miR-121-s whereas miR-121-3p may be referred to as miR-121-as.

The above naming conventions have evolved over time and are general guidelines rather than absolute rules. For example, the let- and lin-families of miRNAs continue to be referred to by these monikers. The mir/miR convention for precursor/mature forms is also a guideline and context should be taken into account to determine which form is referred to. Further details of miR naming can be found at www.mirbase.org or Ambros et al., A uniform system for microRNA annotation, RNA 9:277-279 (2003).

Plant miRNAs follow a different naming convention as described in Meyers et al., Plant Cell. 2008 20(12):3186-3190.

A number of miRNAs are involved in gene regulation, and miRNAs are part of a growing class of non-coding RNAs that is now recognized as a major tier of gene control. In some cases, miRNAs can interrupt translation by binding to regulatory sites embedded in the 3′-UTRs of their target mRNAs, leading to the repression of translation. Target recognition involves complementary base pairing of the target site with the miRNA's seed region (positions 2-8 at the miRNA's 5′ end), although the exact extent of seed complementarity is not precisely determined and can be modified by 3′ pairing. In other cases, miRNAs function like small interfering RNAs (siRNA) and bind to perfectly complementary mRNA sequences to destroy the target transcript.

Characterization of a number of miRNAs indicates that they influence a variety of processes, including early development, cell proliferation and cell death, apoptosis and fat metabolism. For example, some miRNAs, such as lin-4, let-7, mir-14, mir-23, and bantam, have been shown to play critical roles in cell differentiation and tissue development. Others are believed to have similarly important roles because of their differential spatial and temporal expression patterns.

The miRNA database available at miRBase (www.mirbase.org) comprises a searchable database of published miRNA sequences and annotation. Further information about miRBase can be found in the following articles, each of which is incorporated by reference in its entirety herein: Griffiths-Jones et al., miRBase: tools for microRNA genomics. NAR 2008 36(Database Issue):D154-D158; Griffiths-Jones et al., miRBase: microRNA sequences, targets and gene nomenclature. NAR 2006 34(Database Issue):D140-D144; and Griffiths-Jones, S. The microRNA Registry. NAR 2004 32(Database Issue):D109-D111. Representative miRNAs contained in Release 16 of miRBase, made available September 2010.

As described herein, microRNAs are known to be involved in cancer and other diseases and can be assessed in order to characterize a phenotype in a sample. See, e.g., Ferracin et al., Micromarkers: miRNAs in cancer diagnosis and prognosis, Exp Rev Mol Diag, April 2010, Vol. 10, No. 3, Pages 297-308; Fabbri, miRNAs as molecular biomarkers of cancer, Exp Rev Mol Diag, May 2010, Vol. 10, No. 4, Pages 435-444. Techniques to isolate and characterize vesicles and miRs are disclosed herein and/or known to those of skill in the art. In addition to the methodology presented herein, additional methods can be found in U.S. Pat. No. 7,888,035, entitled “METHODS FOR ASSESSING RNA PATTERNS” and issued Feb. 15, 2011; and International Patent Application Nos. PCT/US2010/058461, entitled “METHODS AND SYSTEMS FOR ISOLATING, STORING, AND ANALYZING VESICLES” and filed Nov. 30, 2010; and PCT/US2011/021160, entitled “DETECTION OF GASTROINTESTINAL DISORDERS” and filed Jan. 13, 2011; each of which applications are incorporated by reference herein in their entirety.

### Circulating Biomarkers

Circulating biomarkers include biomarkers that are detectable in body fluids, such as blood, plasma, serum. Examples of circulating cancer biomarkers include cardiac troponin T (cTnT), prostate specific antigen (PSA) for prostate cancer and CA125 for ovarian cancer. Circulating biomarkers according to the invention include any appropriate biomarker that can be detected in bodily fluid, including without limitation protein, nucleic acids, e.g., DNA, mRNA and microRNA, lipids, carbohydrates and metabolites. Circulating biomarkers can include biomarkers that are not associated with cells, such as biomarkers that are membrane associated, embedded in membrane fragments, part of a biological complex, or free in solution. In one embodiment, circulating biomarkers are biomarkers that are associated with one or more vesicles present in the biological fluid of a subject.

Circulating biomarkers have been identified for use in characterization of various phenotypes. See, e.g., Ahmed N, et al., Proteomic-based identification of haptoglobin-1 precursor as a novel circulating biomarker of ovarian cancer. Br. J. Cancer 2004; Mathelin et al., Circulating proteinic biomarkers and breast cancer, Gynecol Obstet Fertil. 2006 July-August; 34(7-8):638-46. Epub 2006 Jul. 28; Ye et al., Recent technical strategies to identify diagnostic biomarkers for ovarian cancer. Expert Rev Proteomics. 2007 Feb. 4(1):121-31; Carney, Circulating oncoproteins HER2/neu, EGFR and CAIX (MN) as novel cancer biomarkers. Expert Rev Mol Diagn. 2007 May; 7(3):309-19; Gagnon, Discovery and application of protein biomarkers for ovarian cancer, Curr Opin Obstet Gynecol. 2008 Feb. 20(1):9-13; Pasterkamp et al., Immune regulatory cells: circulating biomarker factories in cardiovascular disease. Clin Sci (Lond). 2008 August; 115(4):129-31; Fabbri, miRNAs as molecular biomarkers of cancer, Exp Rev Mol Diag, May 2010, Vol. 10, No. 4, Pages 435-444; PCT Patent Publication WO/2007/088537; U.S. Pat. Nos. 7,745,150 and 7,655,479; U.S. Patent Publications 20110008808, 20100330683, 20100248290, 20100222230, 20100203566, 20100173788, 20090291932, 20090239246, 20090226937, 20090111121, 20090004687, 20080261258, 20080213907, 20060003465, 20050124071, and 20040096915, each of which publication is incorporated herein by reference in its entirety.

### Sample Processing

A vesicle or a population of vesicles may be isolated, purified, concentrated or otherwise enriched prior to and/or during analysis. Unless otherwise specified, the terms “purified,” “isolated,” or similar as used herein in reference to vesicles or biomarker components are intended to include partial or complete purification or isolation of such components from a cell or organism. Analysis of a vesicle can include quantitiating the amount one or more vesicle populations of a biological sample. For example, a heterogeneous population of vesicles can be quantitated, or a homogeneous population of vesicles, such as a population of vesicles with a particular biomarker profile, a particular biosignature, or derived from a particular cell type can be isolated from a heterogeneous population of vesicles and quantitated. Analysis of a vesicle can also include detecting, quantitatively or qualitatively, one or more particular biomarker profile or biosignature of a vesicle, as described herein.

A vesicle can be stored and archived, such as in a bio-fluid bank and retrieved for analysis as necessary. A vesicle may also be isolated from a biological sample that has been previously harvested and stored from a living or deceased subject. In addition, a vesicle may be isolated from a biological sample which has been collected as described in King et al., Breast Cancer Res 7(5): 198-204 (2005). A vesicle can be isolated from an archived or stored sample. Alternatively, a vesicle may be isolated from a biological sample and analyzed without storing or archiving of the sample. Furthermore, a third party may obtain or store the biological sample, or obtain or store the vesicle for analysis.

An enriched population of vesicles can be obtained from a biological sample. For example, vesicles may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof.

Size exclusion chromatography, such as gel permeation columns, centrifugation or density gradient centrifugation, and filtration methods can be used. For example, a vesicle can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

Highly abundant proteins, such as albumin and immunoglobulin in blood samples, may hinder isolation of vesicles from a biological sample. For example, a vesicle can be isolated from a biological sample using a system that uses multiple antibodies that are specific to the most abundant proteins found in a biological sample, such as blood. Such a system can remove up to several proteins at once, thus unveiling the lower abundance species such as cell-of-origin specific vesicles. This type of system can be used for isolation of vesicles from biological samples such as blood, cerebrospinal fluid or urine. The isolation of vesicles from a biological sample may also be enhanced by high abundant protein removal methods as described in Chromy et al. J Proteome Res 2004; 3:1120-1127. In another embodiment, the isolation of vesicles from a biological sample may also be enhanced by removing serum proteins using glycopeptide capture as described in Zhang et al, Mol Cell Proteomics 2005; 4:144-155. In addition, vesicles from a biological sample such as urine may be isolated by differential centrifugation followed by contact with antibodies directed to cytoplasmic or anti-cytoplasmic epitopes as described in Pisitkun et al., Proc Natl Acad Sci US A, 2004; 101:13368-13373.

Plasma contains a large variety of proteins including albumin, immunoglobulins, and clotting proteins such as fibrinogen. About 60% of plasma protein comprises the protein albumin (e.g., human serum albumin or HSA), which contributes to osmotic pressure of plasma to assist in the transport of lipids and steroid hormones. Globulins make up about 35% of plasma proteins and are used in the transport of ions, hormones and lipids assisting in immune function. About 4% of plasma protein comprises fibrinogen which is essential in the clotting of blood and can be converted into the insoluble protein fibrin. Other types of blood proteins include: Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin and Mannose-binding lectin (MBL). Any of these proteins, including classes of proteins, or derivatives thereof (such as fibrin which is derived from the cleavage of fibrinogen) can be selectively depleted from a biological sample prior to further analysis performed on the sample. Without being bound by theory, removal of such background proteins may facilitate more sensitive, accurate, or precise detection of the biomarkers of interest in the sample.

Abundant proteins in blood or blood derivatives (e.g., plasma or serum) include without limitation albumin, IgG, transferrin, fibrinogen, IgA, α₂-Macroglobulin, IgM, α₁-Antitrypsin, complement C3, haptoglobulin, apolipoprotein A1, apolipoprotein A3, apolipoprotein B, α₁-Acid Glycoprotein, ceruloplasmin, complement C4, C1q, IgD, prealbumin (transthyretin), and plasminogen. Such proteins can be depleted using commercially available columns and kits. Examples of such columns comprise the Multiple Affinity Removal System from Agilent Technologies (Santa Clara, Calif.). This system include various cartridges designed to deplete different protein profiles, including the following cartridges with performance characteristics according to the manufacturer: Human 14, which eliminates approximately 94% of total protein (albumin, IgG, antitrypsin, IgA, transferrin, haptoglobin, fibrinogen, alpha2-macroglobulin, alpha1-acid glycoprotein (orosomucoid), IgM, apolipoprotein AI, apolipoprotein AII, complement C3 and transthyretin); Human 7, which eliminates approximately 85-90% of total protein (albumin, IgG, IgA, transferrin, haptoglobin, antitrypsin, and fibrinogen); Human 6, which eliminates approximately 85-90% of total protein (albumin, IgG, IgA, transferrin, haptoglobin, and antitrypsin); Human Albumin/IgG, which eliminates approximately 69% of total protein (albumin and IgG); and Human Albumin, which eliminates approximately 50-55% of total protein (albumin). The ProteoPrep® 20 Plasma Immunodepletion Kit from Sigma-Aldrich is intended to specifically remove the 20 most abundant proteins from human plasma or serum, which is about remove 97-98% of the total protein mass in plasma or serum (Sigma-Aldrich, St. Louis, Mo.). According to the manufacturer, the ProteoPrep® 20 removes: albumin, IgG, transferrin, fibrinogen, IgA, α₂-Macroglobulin, IgM, α₁-Antitrypsin, complement C3, haptoglobulin, apolipoprotein A1, A3 and B; α₁-Acid Glycoprotein, ceruloplasmin, complement C4, C1q; IgD, prealbumin, and plasminogen. Sigma-Aldrich also manufactures ProteoPrep® columns to remove albumin (HSA) and immunoglobulins (IgG). The ProteomeLab IgY-12 High Capacity Proteome Partitioning kits from Beckman Coulter (Fullerton, Calif.) are specifically designed to remove twelve highly abundant proteins (Albumin, IgG, Transferrin, Fibrinogen, IgA, α2-macroglobulin, IgM, α₁-Antitrypsin, Haptoglobin, Orosomucoid, Apolipoprotein A-I, Apolipoprotein A-II) from the human biological fluids such as serum and plasma. Generally, such systems rely on immunodepletion to remove the target proteins, e.g., using small ligands and/or full antibodies. The PureProteome™ Human Albumin/Immunoglobulin Depletion Kit from Millipore (EMD Millipore Corporation, Billerica, Mass., USA) is a magnetic bead based kit that enables high depletion efficiency (typically >99%) of Albumin and all Immunoglobulins (i.e., IgG, IgA, IgM, IgE and IgD) from human serum or plasma samples. The ProteoExtract® Albumin/IgG Removal Kit, also from Millipore, is designed to deplete >80% of albumin and IgG from body fluid samples. Other similar protein depletion products include without limitation the following: Aurum™ Affi-Gels Blue mini kit (Bio-Rad, Hercules, Calif., USA); Vivapure® anti-HSA/IgG kit (Sartorius Stedim Biotech, Goettingen, Germany), Qproteome albumin/IgG depletion kit (Qiagen, Hilden, Germany); Seppro® MIXED12-LC20 column (GenWay Biotech, San Diego, Calif., USA); Abundant Serum Protein Depletion Kit (Norgen Biotek Corp., Ontario, Canada); GBC Human Albumin/IgG/Transferrin 3 in 1 Depletion Column/Kit (Good Biotech Corp., Taiwan). These systems and similar systems can be used to remove abundant proteins from a biological sample, thereby improving the ability to detect low abundance circulating biomarkers such as proteins and vesicles.

Thromboplastin is a plasma protein aiding blood coagulation through conversion of prothrombin to thrombin. Thrombin in turn acts as a serine protease that converts soluble fibrinogen into insoluble strands of fibrin, as well as catalyzing many other coagulation-related reactions. Thus, thromboplastin is a protein that can be used to facilitate precipitation of fibrinogen/fibrin (blood clotting factors) out of plasma. In addition to or as an alternative to immunoaffinity protein removal, a blood sample can be treated with thromboplastin to deplete fibrinogen/fibrin. Thromboplastin removal can be performed in addition to or as an alternative to immunoaffinity protein removal as described above using methods known in the art. Precipitation of other proteins and/or other sample particulate can also improve detection of circulating biomarkers such as vesicles in a sample. For example, ammonium sulfate treatment as known in the art can be used to precipitate immunoglobulins and other highly abundant proteins.

In an embodiment, the invention provides a method of detecting a presence or level of one or more circulating biomarker such as a microvesicle in a biological sample, comprising: (a) providing a biological sample comprising or suspected to comprise the one or more circulating biomarker; (b) selectively depleting one or more abundant protein from the biological sample provided in step (a); (c) performing affinity selection of the one or more circulating biomarker from the sample depleted in step (b), thereby detecting the presence or level of one or more circulating biomarker. The biological sample may comprise a bodily fluid, e.g., peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, or a derivative of any thereof. In some embodiments, the biological sample comprises peripheral blood, serum or plasma. See Example 40 herein for illustrative protocols and results from selectively depleting one or more abundant protein from blood plasma prior to vesicle detection.

An abundant protein may comprise a protein in the sample that is present in the sample at a high enough concentration to potentially interfere with downstream processing or analysis. Typically, an abundant protein is not the target of any further analysis of the sample. The abundant protein may constitute at least 10⁻⁵, 10⁻⁴, 10⁻³, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 96, 97, 98 or at least 99% of the total protein mass in the sample. In some embodiments, the abundant protein is present at less than 10⁻⁵% of the total protein mass in the sample, e.g., in the case of a rare target of interest. As described herein, in the case of blood or a derivative thereof, the one or more abundant protein may comprise one or more of albumin, IgG, transferrin, fibrinogen, fibrin, IgA, α2-Marcroglobulin, IgM, α1-Antitrypsin, complement C3, haptoglobulin, apolipoprotein A1, A3 and B; α1-Acid Glycoprotein, ceruloplasmin, complement C4, C1q, IgD, prealbumin (transthyretin), plasminogen, a derivative of any thereof, and a combination thereof. The one or more abundant protein in blood or a blood derivative may also comprise one or more of Albumin, Immunoglobulins, Fibrinogen, Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin, Mannose-binding lectin (MBL), a derivative of any thereof, and a combination thereof.

In some embodiments, selectively depleting the one or more abundant protein comprises contacting the biological sample with thromboplastin to initiate precipitation of fibrin. The one or more abundant protein may also be depleted by immunoaffinity, precipitation, or a combination thereof. For example, the sample can be treated with thromboplastin to precipitate fibrin, and then the sample may be passed through a column to remove HSA, IgG, and other abundant proteins as desired.

“Selectively depleting” the one or more abundant protein comprises depleting the abundant protein from the sample at a higher percentage than depletion another entity in the sample, such as another protein or microvesicle, including a target of interest for downstream processing or analysis. Selectively depleting the one or more abundant protein may comprise depleting the abundant protein at a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10⁴-fold, 10⁵-fold, 10⁶-fold, 10⁷-fold, 10⁸-fold, 10⁹-fold, 10¹⁰-fold, 10¹¹-fold, 10¹²-fold, 10¹³-fold, 10¹⁴-fold, 10¹⁵-fold, 10¹⁶-fold, 10¹⁷-fold, 10¹⁸-fold, 10¹⁹-fold, 10²⁰-fold, or higher rate than another entity in the sample, such as another protein or microvesicle, including a target of interest for downstream processing or analysis. In an embodiment, there is little to no observable depletion of the target of interest as compared to the depletion of the abundant protein. In some embodiments, selectively depleting the one or more abundant protein from the biological sample comprises depleting at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the one or more abundant protein.

Removal of highly abundant proteins and other non-desired entities can further be facilitated with a non-stringent size exclusion step. For example, the sample can be processed using a high molecular weight cutoff size exclusion step to preferentially enrich high molecular weight vesicles apart from lower molecular weight proteins and other entities. In some embodiments, a sample is processed with a column (e.g., a gel filtration column) or filter having a molecular weight cutoff (MWCO) of 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In an embodiment, a 700 kDa filtration column is used. In such a step, the vesicles will be retained or flow more slowly than the column or filter than the lower molecular weight entities. Such columns and filters are known in the art.

Isolation or enrichment of a vesicle from a biological sample can also be enhanced by use of sonication (for example, by applying ultrasound), detergents, other membrane-activating agents, or any combination thereof. For example, ultrasonic energy can be applied to a potential tumor site, and without being bound by theory, release of vesicles from a tissue can be increased, allowing an enriched population of vesicles that can be analyzed or assessed from a biological sample using one or more methods disclosed herein.

With methods of detecting circulating biomarkers as described here, e.g., antibody affinity isolation, the consistency of the results can be optimized as necessary using various concentration or isolation procedures. Such steps can include agitation such as shaking or vortexing, different isolation techniques such as polymer based isolation, e.g., with PEG, and concentration to different levels during filtration or other steps. It will be understood by those in the art that such treatments can be applied at various stages of testing the vesicle containing sample. In one embodiment, the sample itself, e.g., a bodily fluid such as plasma or serum, is vortexed. In some embodiments, the sample is vortexed after one or more sample treatment step, e.g., vesicle isolation, has occurred. Agitation can occur at some or all appropriate sample treatment steps as desired. Additives can be introduced at the various steps to improve the process, e.g., to control aggregation or degradation of the biomarkers of interest.

The results can also be optimized as desireable by treating the sample with various agents. Such agents include additives to control aggregation and/or additives to adjust pH or ionic strength. Additives that control aggregation include blocking agents such as bovine serum albumin (BSA), milk or StabilGuard® (a BSA-free blocking agent; Product code SG02, Surmodics, Eden Prairie, Minn.), chaotropic agents such as guanidium hydro chloride, and detergents or surfactants. Useful ionic detergents include sodium dodecyl sulfate (SDS, sodium lauryl sulfate (SLS)), sodium laureth sulfate (SLS, sodium lauryl ether sulfate (SLES)), ammonium lauryl sulfate (ALS), cetrimonium bromide, cetrimonium chloride, cetrimonium stearate, and the like. Useful non-ionic (zwitterionic) detergents include polyoxyethylene glycols, polysorbate 20 (also known as Tween 20), other polysorbates (e.g., 40, 60, 65, 80, etc), Triton-X (e.g., X100, X114), 3-[(3-cholamidopropyfidimethylammonio]-1-propanesulfonate (CHAPS), CHAPSO, deoxycholic acid, sodium deoxycholate, NP-40, glycosides, octyl-thio-glucosides, maltosides, and the like. In some embodiments, Pluronic F-68, a surfactant shown to reduce platelet aggregation, is used to treat samples containing vesicles during isolation and/or detection. F68 can be used from a 0.1% to 10% concentration, e.g., a 1%, 2.5% or 5% concentration. The pH and/or ionic strength of the solution can be adjusted with various acids, bases, buffers or salts, including without limitation sodium chloride (NaCl), phosphate-buffered saline (PBS), tris-buffered saline (TBS), sodium phosphate, potassium chloride, potassium phosphate, sodium citrate and saline-sodium citrate (SSC) buffer. In some embodiments, NaCl is added at a concentration of 0.1% to 10%, e.g., 1%, 2.5% or 5% final concentration. In some embodiments, Tween 20 is added to 0.005 to 2% concentration, e.g., 0.05%, 0.25% or 0.5% final concentration. Blocking agents for use with the invention comprise inert proteins, e.g., milk proteins, non-fat dry milk protein, albumin, BSA, casein, or serum such as newborn calf serum (NBCS), goat serum, rabbit serum or salmon serum. The proteins can be added at a 0.1% to 10% concentration, e.g., 1%, 2%, 3%, 3.5%, 4%, 5%, 6%, 7%, 8%, 9% or 10% concentration. In some embodiments, BSA is added to 0.1% to 10% concentration, e.g., 1%, 2%, 3%, 3.5%, 4%, 5%, 6%, 7%, 8%, 9% or 10% concentration. In an embodiment, the sample is treated according to the methodology presented in U.S. patent application Ser. No. 11/632,946, filed Jul. 13, 2005, which application is incorporated herein by reference in its entirety. Commercially available blockers may be used, such as SuperBlock, StartingBlock, Protein-Free from Pierce (a division of Thermo Fisher Scientific, Rockford, Ill.). In some embodiments, SSC/detergent (e.g., 20×SSC with 0.5% Tween 20 or 0.1% Triton-X 100) is added to 0.1% to 10% concentration, e.g., at 1.0% or 5.0% concentration.

The methods of detecting vesicles and other circulating biomarkers can be optimized as desired with various combinations of protocols and treatments as described herein. A detection protocol can be optimized by various combinations of agitation, isolation methods, and additives. In some embodiments, the patient sample is vortexed before and after isolation steps, and the sample is treated with blocking agents including BSA and/or F68. Such treatments may reduce the formation of large aggregates or protein or other biological debris and thus provide a more consistent detection reading.

### Filtration and Ultrafiltration

A vesicle can be isolated from a biological sample by filtering a biological sample from a subject through a filtration module and collecting from the filtration module a retentate comprising the vesicle, thereby isolating the vesicle from the biological sample. The method can comprise filtering a biological sample from a subject through a filtration module comprising a filter (also referred to herein as a selection membrane); and collecting from the filtration module a retentate comprising the vesicle, thereby isolating the vesicle from the biological sample. For example, in one embodiment, the filter retains molecules greater than about 100 kiloDaltons. In such cases, microvesicles are generally found within the retentate of the filtration process whereas smaller entities such as proteins, protein complexes, nucleic acids, etc, pass through into the filtrate.

The method can be used when determining a biosignature of one or more microvesicle. The method can also further comprise contacting the retentate from the filtration to a plurality of substrates, wherein each substrate is coupled to one or more capture agents, and each subset of the plurality of substrates comprises a different capture agent or combination of capture agents than another subset of the plurality of substrates.

Also provided herein is a method of determining a biosignature of a vesicle in a sample comprising: filtering a biological sample from a subject with a disorder through a filtration module, collecting from the filtration module a retentate comprising one or more vesicles, and determining a biosignature of the one or more vesicles. In one embodiment, the filtration module comprises a filter that retains molecules greater than about 100 or 150 kiloDaltons.

The method disclosed herein can further comprise characterizing a phenotype in a subject by filtering a biological sample from a subject through a filtration module, collecting from the filtration module a retentate comprising one or more vesicles; detecting a biosignature of the one or more vesicles; and characterizing a phenotype in the subject based on the biosignature, wherein characterizing is with at least 70% sensitivity. In some embodiments, characterizing comprises determining an amount of one or more vesicle having the biosignature. Furthermore, the characterizing can be from about 80% to 100% sensitivity.

Also provided herein is a method for multiplex analysis of a plurality of vesicles. In some embodiments, the method comprises filtering a biological sample from a subject through a filtration module; collecting from the filtration module a retentate comprising the plurality of vesicles, applying the plurality of vesicles to a plurality of capture agents, wherein the plurality of capture agents is coupled to a plurality of substrates, and each subset of the plurality of substrates is differentially labeled from another subset of the plurality of substrates; capturing at least a subset of the plurality of vesicles; and determining a biosignature for at least a subset of the captured vesicles. In one embodiment, each substrate is coupled to one or more capture agents, and each subset of the plurality of substrates comprises a different capture agent or combination of capture agents as compared to another subset of the plurality of substrates. In some embodiments, at least a subset of the plurality of substrates is intrinsically labeled, such as comprising one or more labels. The substrate can be a particle or bead, or any combination thereof. In some embodiments, the filter retains molecules greater than 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In one embodiment, the filtration module comprises a filter that retains molecules greater than about 100 or 150 kiloDaltons. In one embodiment, the filtration module comprises a filter that retains molecules greater than about 9, 20, 100 or 150 kiloDaltons. In still another embodiment, the filtration module comprises a filter that retains molecules greater than about 7000 kDa.

In some embodiments, the method for multiplex analysis of a plurality of vesicles comprises filtering a biological sample from a subject through a filtration module, wherein the filtration module comprises a filter that retains molecules greater than about 100 kiloDaltons; collecting from the filtration module a retentate comprising the plurality of vesicles; applying the plurality of vesicles to a plurality of capture agents, wherein the plurality of capture agents is coupled to a microarray; capturing at least a subset of the plurality of vesicles on the microarray; and determining a biosignature for at least a subset of the captured vesicles. In some embodiments, the filter retains molecules greater than 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In one embodiment, the filtration module comprises a filter that retains molecules greater than about 100 or 150 kiloDaltons. In one embodiment, the filtration module comprises a filter that retains molecules greater than about 9, 20, 100 or 150 kiloDaltons. In still another embodiment, the filtration module comprises a filter that retains molecules greater than about 7000 kDa.

The biological sample can be clarified prior to isolation by filtration. Clarification comprises selective removal of cellular debris and other undesirable materials. For example, cellular debris and other components that may interfere with detection of the circulating biomarkers can be removed. The clarification can be by low-speed centrifugation, such as at about 5,000×g, 4,000×g, 3,000×g, 2,000×g, 1,000×g, or less. The supernatant, or clarified biological sample, containing the vesicle can then be collected and filtered to isolate the vesicle from the clarified biological sample. In some embodiments, the biological sample is not clarified prior to isolation of a vesicle by filtration.

In some embodiments, isolation of a vesicle from a sample does not use high-speed centrifugation, such as ultracentrifugation. For example, isolation may not require the use of centrifugal speeds, such as about 100,000×g or more. In some embodiments, isolation of a vesicle from a sample uses speeds of less than 50,000×g, 40,000×g, 30,000×g, 20,000×g, 15,000×g, 12,000×g, or 10,000×g.

Any number of applicable filter configurations can be used to filter a sample of interest. In some embodiments, the filtration module used to isolate the circulating biomarkers from the biological sample is a fiber-based filtration cartridge. For example, the fiber can be a hollow polymeric fiber, such as a polypropylene hollow fiber. A biological sample can be introduced into the filtration module by pumping the sample fluid, such as a biological fluid as disclosed herein, into the module with a pump device, such as a peristaltic pump. The pump flow rate can vary, such as at about 0.25, 0.5, 1, 1.5, 2, 2.5, 3, 3.5, 4, 4.5, 5, 6, 7, 8, 9, or 10 mL/minute. The flow rate can be adjusted given the configuration, e.g., size and throughput, of the filtration module.

The filtration module can be a membrane filtration module. For example, the membrane filtration module can comprise a filter disc membrane, such as a hydrophilic polyvinylidene difluoride (PVDF) filter disc membrane housed in a stirred cell apparatus (e.g., comprising a magnetic stirrer). In some embodiments, the sample moves through the filter as a result of a pressure gradient established on either side of the filter membrane.

The filter can comprise a material having low hydrophobic absorptivity and/or high hydrophilic properties. For example, the filter can have an average pore size for vesicle retention and permeation of most proteins as well as a surface that is hydrophilic, thereby limiting protein adsorption. For example, the filter can comprise a material selected from the group consisting of polypropylene, PVDF, polyethylene, polyfluoroethylene, cellulose, secondary cellulose acetate, polyvinylalcohol, and ethylenevinyl alcohol (EVAL®, Kuraray Co., Okayama, Japan). Additional materials that can be used in a filter include, but are not limited to, polysulfone and polyethersulfone.

The filtration module can have a filter that retains molecules greater than about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, or 900 kiloDaltons (kDa), such as a filter that has a MWCO (molecular weight cut off) of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, or 900 kDa, respectively. In embodiments, the filtration module has a MWCO of 1000 kDa, 1500 kDa, 2000 kDa, 2500 kDa, 3000 kDa, 3500 kDa, 4000 kDa, 4500 kDa, 5000 kDa, 5500 kDa, 6000 kDa, 6500 kDa, 7000 kDa, 7500 kDa, 8000 kDa, 8500 kDa, 9000 kDa, 9500 kDa, 10000 kDa, or greater than 10000 kDa. Ultrafiltration membranes with a range of MWCO of 9 kDa, 20 kDa and/or 150 kDa can be used. In some embodiments, the filter within the filtration module has an average pore diameter of about 0.01 μm to about 0.15 μm, and in some embodiments from about 0.05 μm to about 0.12 μm. In some embodiments, the filter has an average pore diameter of about 0.06 μm, 0.07 μm, 0.08 μm, 0.09 μm, 0.1 μm, 0.11 μm or 0.2 μm.

The filtration module can be a commerically available column, such as a column typically used for concentrating proteins or for isolating proteins (e.g., ultrafiltration). Examples include, but are not limited to, columns from Millpore (Billerica, Mass.), such as Amicon® centrifugal filters, or from Pierce® (Rockford, Ill.), such as Pierce Concentrator filter devices. Useful columns from Pierce include disposable ultrafiltration centrifugal devices with a MWCO of 9 kDa, 20 kDa and/or 150 kDa. These concentrators consist of a high-performance regenerated cellulose membrane welded to a conical device. The filters can be as described in U.S. Pat. No. 6,269,957 or 6,357,601, both of which applications are incorporated by reference in their entirety herein.

The retentate comprising the isolated vesicle can be collected from the filtration module. The retentate can be collected by flushing the retentate from the filter. Selection of a filter composition having hydrophilic surface properties, thereby limiting protein adsorption, can be used, without being bound by theory, for easier collection of the retentate and minimize use of harsh or time-consuming collection techniques.

The collected retentate can then be used subsequent analysis, such as assessing a biosignature of one or more vesicles in the retentate, as further described herein. The analysis can be directly performed on the collected retentate. Alternatively, the collected retentate can be further concentrated or purified, prior to analysis of one or more vesicles. For example, the retentate can be further concentrated or vesicles further isolated from the retentate using size exclusion chromatography, density gradient centrifugation, differential centrifugation, immunoabsorbent capture, affinity purification, microfluidic separation, or combinations thereof, such as described herein. In some embodiments, the retentate can undergo another step of filtration. Alternatively, prior to isolation of a vesicle using a filter, the vesicle is concentrated or isolated using techniques including without limitation size exclusion chromatography, density gradient centrifugation, differential centrifugation, immunoabsorbent capture, affinity purification, microfluidic separation, or combinations thereof.

Combinations of filters can be used for concentrating and isolating biomarkers. For example, the biological sample may first be filtered through a filter having a porosity or pore size of between about 0.01 μm to about 10 μm, e.g., 0.01 μm to about 2 μm or about 0.05 μm to about 1.5 μm, and then the sample is filtered. For example, prior to filtering a biological sample through a filtration module with a filter that retains molecules greater than about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa), such as a filter that has a MWCO (molecular weight cut off) of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kDa, respectively, the biological sample may first be filtered through a filter having a porosity or pore size of between about 0.01 μm to about 10 μm, e.g., 0.01 μm to about 2 μm or about 0.05 μm to about 1.5 μm. In some embodiments, the filter has a pore size of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, 3.0, 4.0, 5.0, 6.0, 7.0, 8.0, 9.0 or 10.0 μm. The filter may be a syringe filter. Thus, in one embodiment, the method comprises filtering the biological sample through a filter, such as a syringe filter, wherein the syringe filter has a porosity of greater than about 1 μm, prior to filtering the sample through a filtration module comprising a filter that retains molecules greater than about 100 or 150 kiloDaltons. In an embodiment, the filter is 1.2 μM filter and the filtration is followed by passage of the sample through a 7 ml or 20 ml concentrator column with a 150 kDa cutoff. Multiple concentrator columns may be used, e.g., in series. For example, a 7000 MWCO filtration unit can be used before a 150 MWCO unit.

The filtration module can be a component of a microfluidic device. Microfluidic devices, which may also be referred to as “lab-on-a-chip” systems, biomedical micro-electro-mechanical systems (bioMEMs), or multicomponent integrated systems, can be used for isolating, and analyzing, vesicles. Such systems miniaturize and compartmentalize processes that allow for binding of vesicles, detection of biomarkers, and other processes, such as further described herein.

The filtration module and assessment can be as described in Grant, R., et al., A filtration-based protocol to isolate human Plasma Membrane-derived Vesicles and exosomes from blood plasma, J Immunol Methods (2011) 371:143-51 (Epub 2011 Jun. 30), which reference is incorporated herein by reference in its entirety.

A microfluidic device can also be used for isolation of a vesicle by comprising a filtration module. For example, a microfluidic device can use one more channels for isolating a vesicle from a biological sample based on size from a biological sample. A biological sample can be introduced into one or more microfluidic channels, which selectively allows the passage of vesicles. The microfluidic device can further comprise binding agents, or more than one filtration module to select vesicles based on a property of the vesicles, for example, size, shape, deformability, biomarker profile, or biosignature.

The retentate from a filtration step can be further processed before assessment of microvesicles or other biomarkers therein. In an embodiment, the retentate is diluted prior to biomarker assessment, e.g., with an appropriate diluent such as a biologically compatible buffer. In some cases, the retentate is serially diluted. In an aspect, the invention provides a method for detecting a microvesicle population from a biological sample comprising: a) concentrating the biological sample using a selection membrane having a pore size of from 0.01 μnm to about 10 μm, or a molecular weight cut off (MWCO) from about 1 kDa to 10000 kDa; b) diluting a retentate from the concentration step into one or more aliquots; and c) contacting each of the one or more aliquots of retentate with one or more binding agent specific to a molecule of at least one microvesicle in the microvesicle population. In a related aspect, the invention provides a method for detecting a microvesicle population from a biological sample comprising: a) concentrating the biological sample using a selection membrane having a pore size of from 0.01 μm to about 10 μm, or a molecular weight cut off (MWCO) from about 1 kDa to 10000 kDa; and b) contacting one or more aliquots of the retentate from the concentrating step with one or more binding agent specific to a molecule of at least one microvesicle in the microvesicle population.

The selection membrane can be sized to retain the desired biomarkers in the retentate or to allow the desired biomarkers to pass through the filter into the filtrate. The filter membrane can be chosen to have a certain pore size or MWCO value. The selection membrane can have a pore size of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, 3.0, 4.0, 5.0, 6.0, 7.0, 8.0, 9.0 or 10.0 μm. The selection membrane can also have a MWCO of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 2000, 3000, 4000, 5000, 6000, 7000, 8000, 9000 or 10000 kDa.

The retentate can be separated and/or diluted into any number of desired aliquots. For example, multiple aliquots without any dilution or the same dilution can be used to determine reproducibility. In another example, multiple aliquots at different dilutions can be used to construct a concentration curve. In an embodiment, the retentate is separated and/or diluted into at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350 or 400 aliquots. The aliquots can be at a same dilution or at different dilutions.

A dilution factor is the ratio of the final volume of a mixture (the mixture of the diluents and aliquot) divided by the initial volume of the aliquot. The retentate can be diluted into one or more aliquots at a dilution factor of about 0, 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000 and/or 100000. For example, the retentate can be diluted into one or more aliquot at a dilution factor of about 500.

To estimate a concentration or form a curve, the retentate can be diluted into multiple aliquots. In an embodiment of the method, the retentate is diluted into one or more aliquots at a dilution factor of about 100, 250, 500, 1000, 10000 and 100000. As desired, the method can further comprise detecting an amount of microvesicles in each aliquot of retentate, e.g., that formed a complex with the one or more binding agent. The curve can be used to determine a linear range of the amount of microvesicles in each aliquot detected versus dilution factor. A concentration of the detected microvesicles for the biological sample can be determined using the amount of microvesicles determined in one or more aliquot within the linear range. The concentration can be compared to a reference concentration, e.g., in order to characterize a phenotype as described herein.

The invention also provides a related method comprising filtering a biological sample from a subject through a filtration module and collecting a filtrate comprising the vesicle, thereby isolating the vesicle from the biological sample. In such cases cells and other large entities can be retained in the retentate while microvesicles pass through into the filtrate. It will be appreciated that strategies to retain and filter microvesicles can be used in concert. For example, a sample can be filtered with a selection membrane that allows microvesicles to pass through, thereby isolating the microvesicles from large particles (cells, complexes, etc). The filtrate comprising the microvesicle can then be filtered using a selection membrane that retains microvesicles, thereby isolating the microvesicles from smaller particles (proteins, nucleic acids, etc). The isolated microvesicles can be further assessed according to the methods of the invention, e.g., to characterize a phenotype.

### Precipitation

Vesicles can be isolated using a polymeric precipitation method. The method can be in combination with or in place of the other isolation methods described herein. In one embodiment, the sample containing the vesicles is contacted with a formulation of polyethylene glycol (PEG). The polymeric formulation is incubated with the vesicle containing sample then precipitated by centrifugation. The PEG can bind to the vesicles and can be treated to specifically capture vesicles by addition of a capture moiety, e.g., a pegylated-binding protein such as an antibody. One of skill will appreciate that other polymers in addition to PEG can be used, e.g., PEG derivatives including methoxypolyethylene glycols, poly (ethylene oxide), and various polymers of formula HO—CH₂—(CH₂—O—CH₂-)n-CH₂—OH having different molecular weights.

In some embodiments of the invention, the vesicles are concentrated from a sample using the polymer precipitation method and the isolated vesicles are further separated using another approach. The second step can be used to identify a subpopulation of vesicles, e.g., that display certain biomarkers. The second separation step can comprise size exclusion, a binding agent, an antibody capture step, microbeads, as described herein.

In an embodiment, vesicles are isolated according to the ExoQuick™ and ExoQuick-TC™ kits from System Biosciences, Mountain View, Calif. USA. These kits use a polymer-based precipitation method to pellet vesicles. Similarly, the vesicles can be isolated using the Total Exosome Isolation (from Serum) or Total Exosome Isolation (from Cell Culture Media) kits from Invitrogen/Life Technologies (Carlsbad, Calif. USA). The Total Exosome Isolation reagent forces less-soluble components such as vesicles out of solution, allowing them to be collected by a short, low-speed centrifugation. The reagent is added to the biological sample, and the solution is incubated overnight at 2° C. to 8° C. The precipitated vesicles are recovered by standard centrifugation.

### Binding Agents

Binding agents (also referred to as binding reagents) include agents that are capable of binding a target biomarker. A binding agent can be specific for the target biomarker, meaning the agent is capable of binding a target biomarker. The target can be any useful biomarker disclosed herein, such as a biomarker on the vesicle surface. In some embodiments, the target is a single molecule, such as a single protein, so that the binding agent is specific to the single protein. In other embodiments, the target can be a group of molecules, such as a family or proteins having a similar epitope or moiety, so that the binding agent is specific to the family or group of proteins. The group of molecules can also be a class of molecules, such as protein, DNA or RNA. The binding agent can be a capture agent used to capture a vesicle by binding a component or biomarker of a vesicle. In some embodiments, a capture agent comprises an antibody or fragment thereof, or an aptamer, that binds to an antigen on a vesicle. The capture agent can be optionally coupled to a substrate and used to isolate a vesicle, as further described herein.

A binding agent is an agent that binds to a circulating biomarker, such as a vesicle or a component of a vesicle. The binding agent can be used as a capture agent and/or a detection agent. A capture agent can bind and capture a circulating biomarker, such as by binding a component or biomarker of a vesicle. For example, the capture agent can be a capture antibody or capture antigen that binds to an antigen on a vesicle. A detection agent can bind to a circulating biomarker thereby facilitating detection of the biomarker. For example, a capture agent comprising an antibody or aptamer that is sequestered to a substrate can be used to capture a vesicle in a sample, and a detection agent comprising an antibody or aptamer that carries a label can be used to detect the captured vesicle via detection of the detection agent's label. In some embodiments, a vesicle is assessed using capture and detection agents that recognize the same vesicle biomarkers. For example, a vesicle population can be captured using a tetraspanin such as by using an anti-CD9 antibody bound to a substrate, and the captured vesicles can be detected using a fluorescently labeled anti-CD9 antibody to label the captured vesicles. In other embodiments, a vesicle is assessed using capture and detection agents that recognize different vesicle biomarkers. For example, a vesicle population can be captured using a cell-specific marker such as by using an anti-PCSA antibody bound to a substrate, and the captured vesicles can be detected using a fluorescently labeled anti-CD9 antibody to label the captured vesicles. Similarly, the vesicle population can be captured using a general vesicle marker such as by using an anti-CD9 antibody bound to a substate, and the captured vesicles can be detected using a fluorescently labeled antibody to a cell-specific or disease specific marker to label the captured vesicles.

The biomarkers recognized by the binding agent are sometimes referred to herein as an antigen. Unless otherwise specified, antigen as used herein is meant to encompass any entity that is capable of being bound by a binding agent, regardless of the type of binding agent or the immunogenicity of the biomarker. The antigen further encompasses a functional fragment thereof. For example, an antigen can encompass a protein biomarker capable of being bound by a binding agent, including a fragment of the protein that is capable of being bound by a binding agent.

In one embodiment, a vesicle is captured using a capture agent that binds to a biomarker on a vesicle. The capture agent can be coupled to a substrate and used to isolate a vesicle, as further described herein. In one embodiment, a capture agent is used for affinity capture or isolation of a vesicle present in a substance or sample.

A binding agent can be used after a vesicle is concentrated or isolated from a biological sample. For example, a vesicle can first be isolated from a biological sample before a vesicle with a specific biosignature is isolated or detected. The vesicle with a specific biosignature can be isolated or detected using a binding agent for the biomarker. A vesicle with the specific biomarker can be isolated or detected from a heterogeneous population of vesicles. Alternatively, a binding agent may be used on a biological sample comprising vesicles without a prior isolation or concentration step. For example, a binding agent is used to isolate or detect a vesicle with a specific biosignature directly from a biological sample.

A binding agent can be a nucleic acid, protein, or other molecule that can bind to a component of a vesicle. The binding agent can comprise DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), dendrimers, or a combination thereof. For example, the binding agent can be a capture antibody. In embodiments of the invention, the binding agent comprises a membrane protein labeling agent. See, e.g., the membrane protein labeling agents disclosed in Alroy et al., US. Patent Publication US 2005/0158708. In an embodiment, vesicles are isolated or captured as described herein, and one or more membrane protein labeling agent is used to detect the vesicles.

In some instances, a single binding agent can be employed to isolate or detect a vesicle. In other instances, a combination of different binding agents may be employed to isolate or detect a vesicle. For example, at least 2, 3, 4, 5, 6, 7, 8, 9, 10, 11, 12, 13, 14, 15, 16, 17, 18, 19, 20, 25, 50, 75 or 100 different binding agents may be used to isolate or detect a vesicle from a biological sample. Furthermore, the one or more different binding agents for a vesicle can form a biosignature of a vesicle, as further described below.

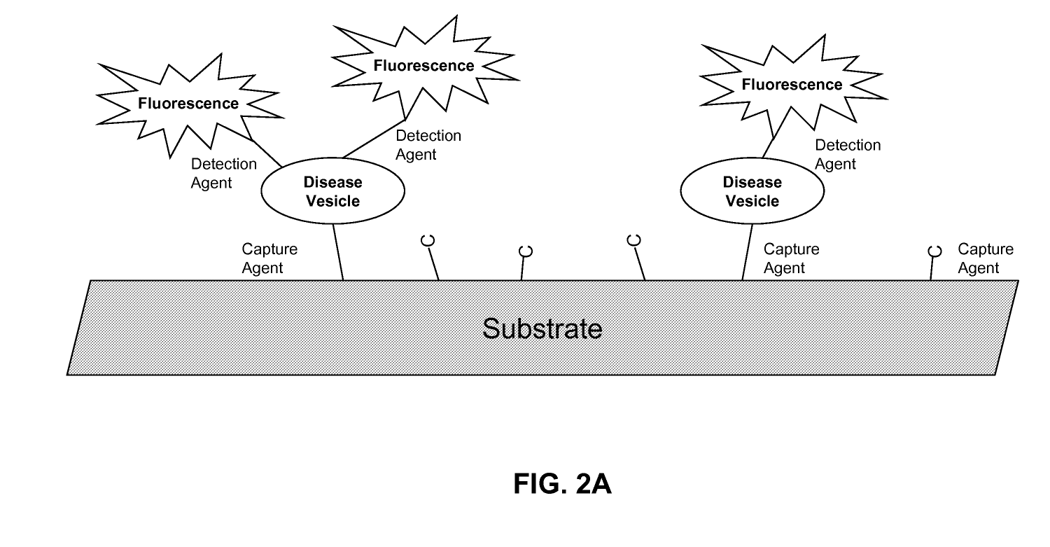

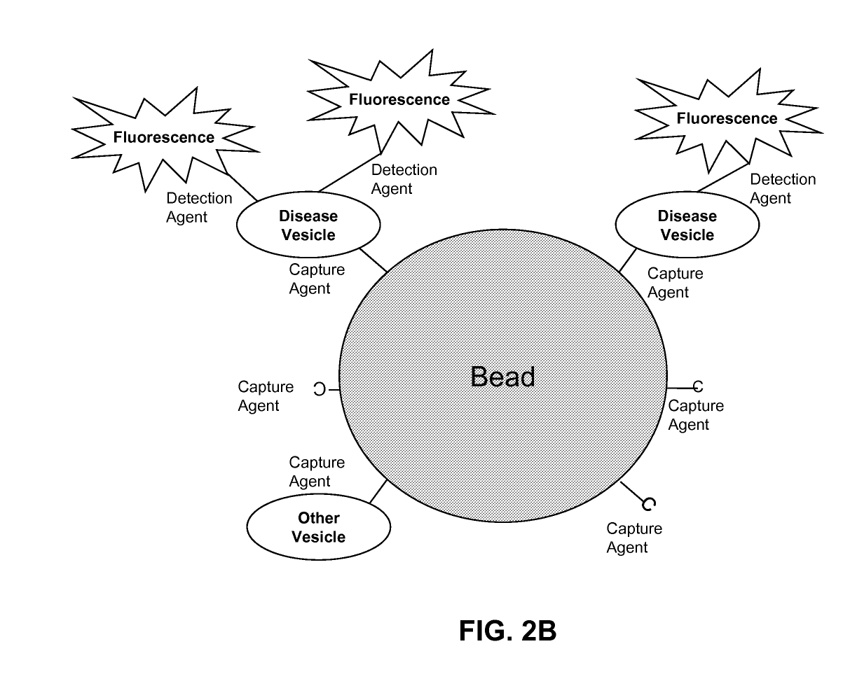

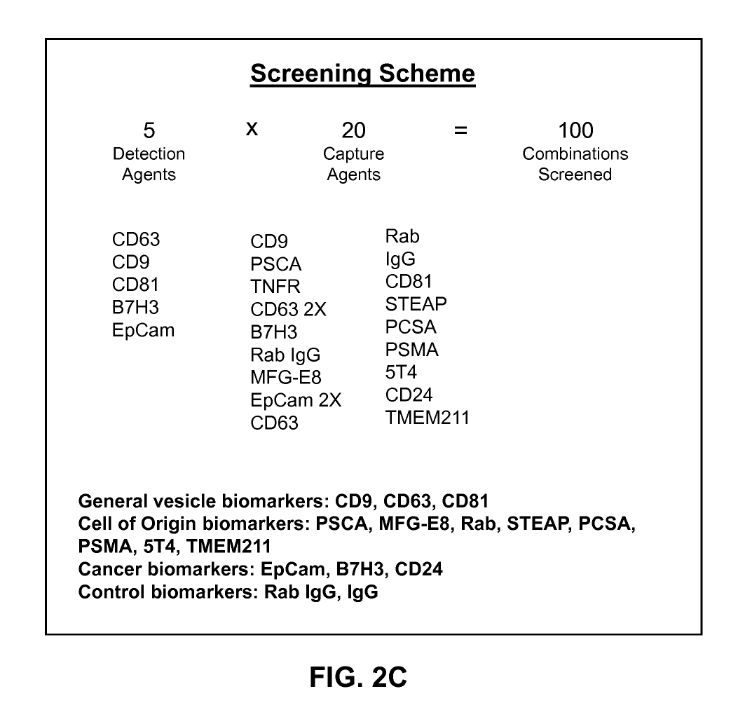

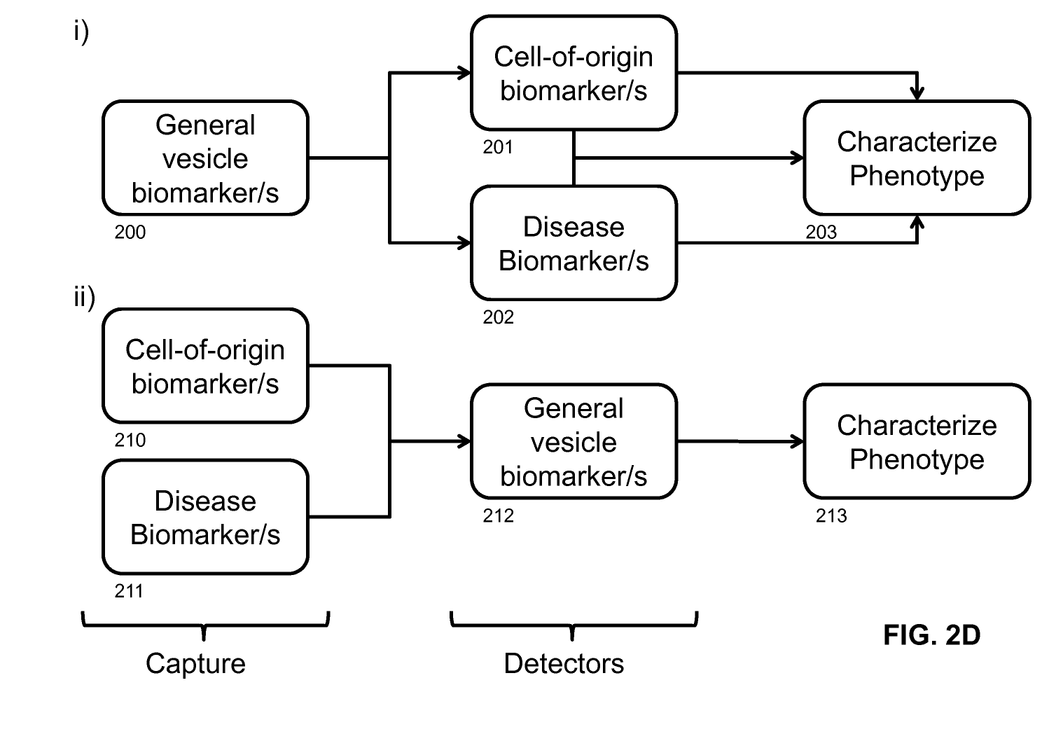

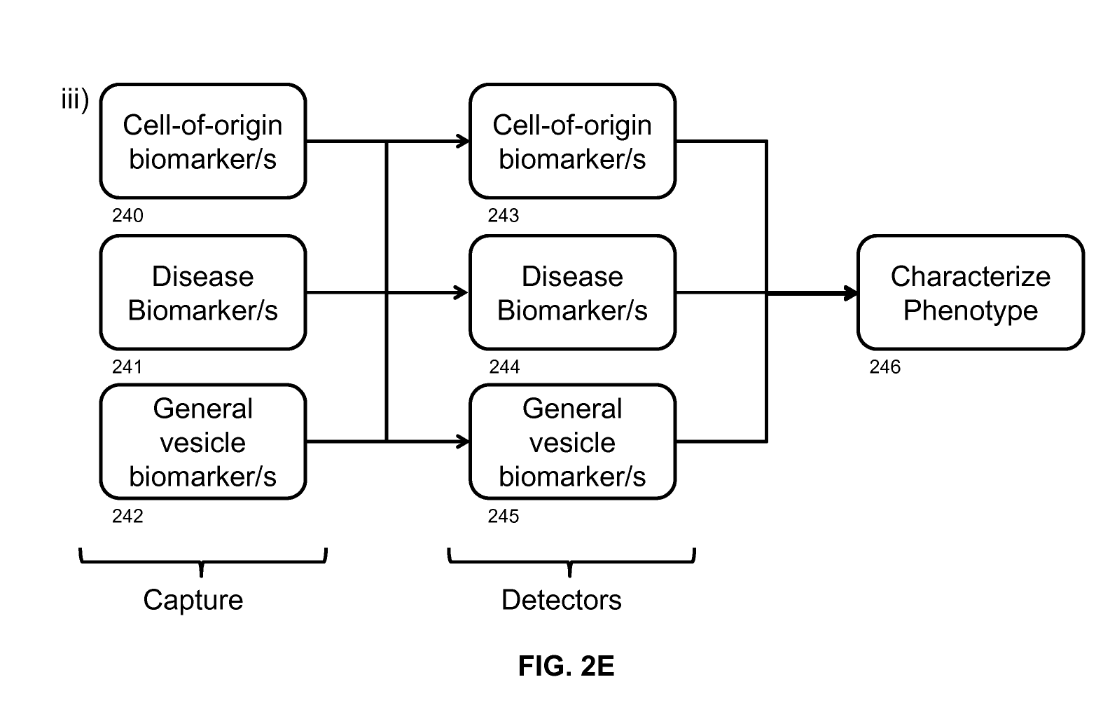

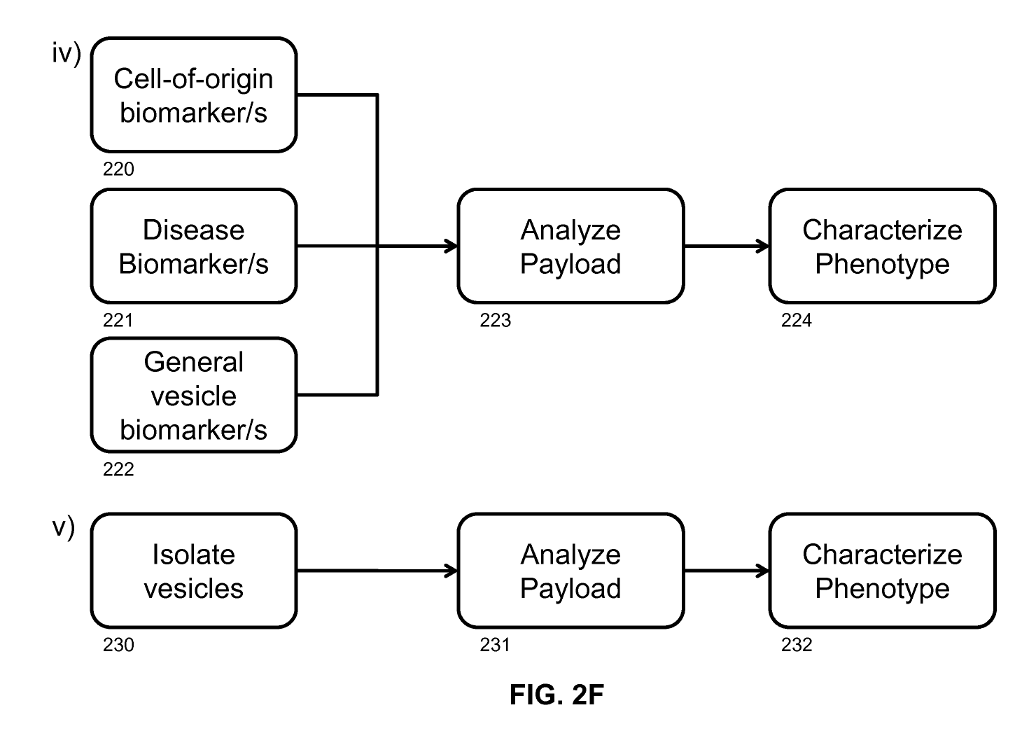

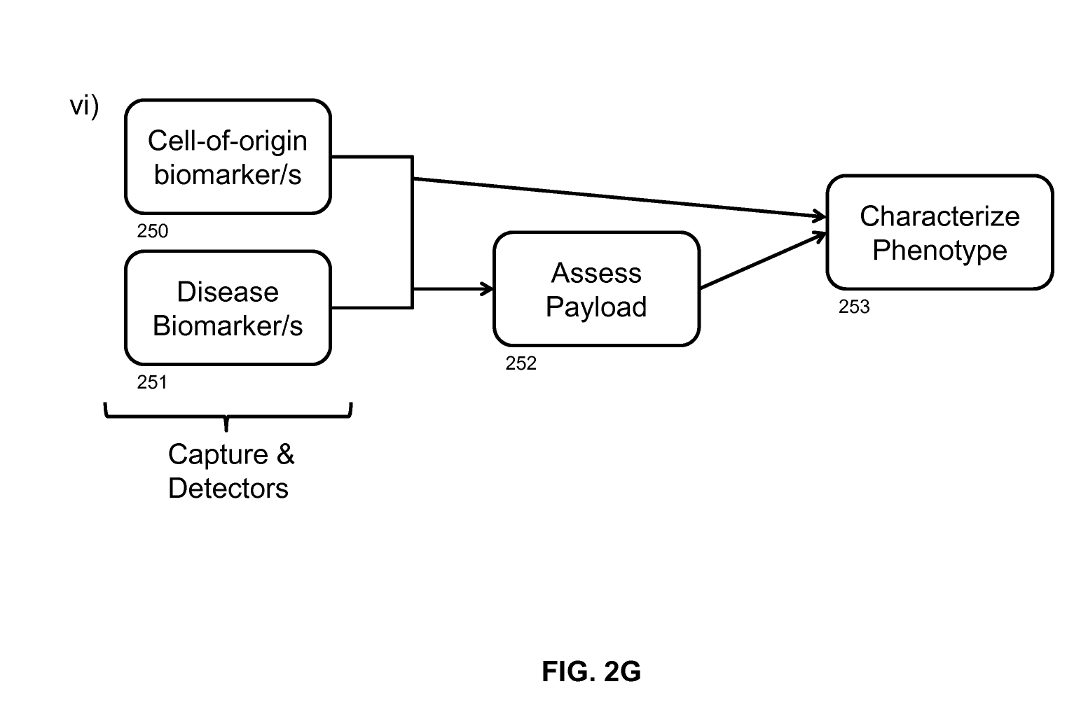

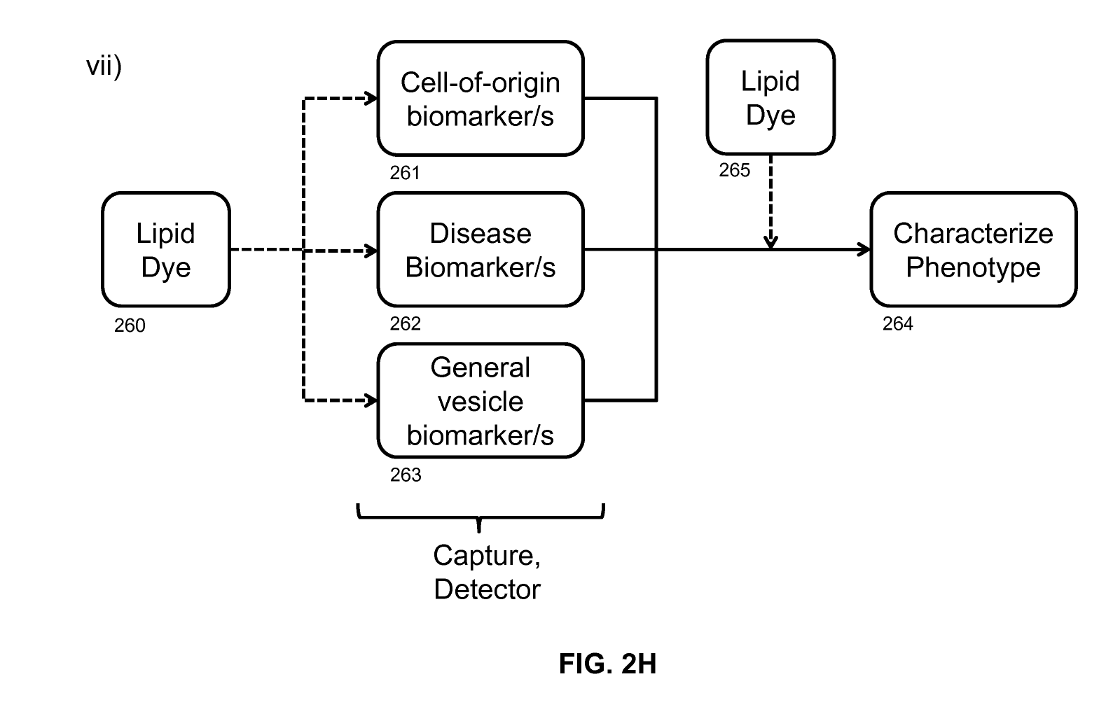

Different binding agents can also be used for multiplexing. For example, isolation or detection of more than one population of vesicles can be performed by isolating or detecting each vesicle population with a different binding agent. Different binding agents can be bound to different particles, wherein the different particles are labeled. In another embodiment, an array comprising different binding agents can be used for multiplex analysis, wherein the different binding agents are differentially labeled or can be ascertained based on the location of the binding agent on the array. Multiplexing can be accomplished up to the resolution capability of the labels or detection method, such as described below. The binding agents can be used to detect the vesicles, such as for detecting cell-of-origin specific vesicles. A binding agent or multiple binding agents can themselves form a binding agent profile that provides a biosignature for a vesicle. One or more binding agents can be selected from FIG. 2 of International Patent Application Serial No. PCT/US2011/031479, entitled “Circulating Biomarkers for Disease” and filed Apr. 6, 2011, which application is incorporated by reference in its entirety herein. For example, if a vesicle population is detected or isolated using two, three, four or more binding agents in a differential detection or isolation of a vesicle from a heterogeneous population of vesicles, the particular binding agent profile for the vesicle population provides a biosignature for the particular vesicle population. The vesicle can be detected using any number of binding agents in a multiplex fashion. Thus, the binding agent can also be used to form a biosignature for a vesicle. The biosignature can be used to characterize a phenotype.

The binding agent can be a lectin. Lectins are proteins that bind selectively to polysaccharides and glycoproteins and are widely distributed in plants and animals. For example, lectins such as those derived from Galanthus nivalis in the form of Galanthus nivalis agglutinin (“GNA”), Narcissus pseudonarcissus in the form of Narcissus pseudonarcissus agglutinin (“NPA”) and the blue green algae Nostoc ellipsosporum called “cyanovirin” (Boyd et al. Antimicrob Agents Chemother 41(7): 1521 1530, 1997; Hammar et al. Ann N Y Acad Sci 724: 166 169, 1994; Kaku et al. Arch Biochem Biophys 279(2): 298 304, 1990) can be used to isolate a vesicle. These lectins can bind to glycoproteins having a high mannose content (Chervenak et al. Biochemistry 34(16): 5685 5695, 1995). High mannose glycoprotein refers to glycoproteins having mannose-mannose linkages in the form of α-1→3 or α-1→6 mannose-mannose linkages.

The binding agent can be an agent that binds one or more lectins. Lectin capture can be applied to the isolation of the biomarker cathepsin D since it is a glycosylated protein capable of binding the lectins Galanthus nivalis agglutinin (GNA) and concanavalin A (ConA).

Methods and devices for using lectins to capture vesicles are described in International Patent Applications PCT/US2010/058461, entitled “METHODS AND SYSTEMS FOR ISOLATING, STORING, AND ANALYZING VESICLES” and filed Nov. 30, 2010; PCT/US2009/066626, entitled “AFFINITY CAPTURE OF CIRCULATING BIOMARKERS” and filed Dec. 3, 2009; PCT/US2010/037467, entitled “METHODS AND MATERIALS FOR ISOLATING EXOSOMES” and filed Jun. 4, 2010; and PCT/US2007/006101, entitled “EXTRACORPOREAL REMOVAL OF MICROVESICULAR PARTICLES” and filed Mar. 9, 2007, each of which applications is incorporated by reference herein in its entirety.

The binding agent can be an antibody. For example, a vesicle may be isolated using one or more antibodies specific for one or more antigens present on the vesicle. For example, a vesicle can have CD63 on its surface, and an antibody, or capture antibody, for CD63 can be used to isolate the vesicle. Alternatively, a vesicle derived from a tumor cell can express EpCam, the vesicle can be isolated using an antibody for EpCam and CD63. Other antibodies for isolating vesicles can include an antibody, or capture antibody, to CD9, PSCA, TNFR, CD63, B7H3, MFG-E8, EpCam, Rab, CD81, STEAP, PCSA, PSMA, or 5T4. Other antibodies for isolating vesicles can include an antibody, or capture antibody, to DR3, STEAP, epha2, TMEM211, MFG-E8, Tissue Factor (TF), unc93A, A33, CD24, NGAL, EpCam, MUC17, TROP2, or TETS.

In some embodiments, the capture agent is an antibody to CD9, CD63, CD81, PSMA, PCSA, B7H3, EpCam, PSCA, ICAM, STEAP, or EGFR. The capture agent can also be used to identify a biomarker of a vesicle. For example, a capture agent such as an antibody to CD9 would identify CD9 as a biomarker of the vesicle. In some embodiments, a plurality of capture agents can be used, such as in multiplex analysis. The plurality of captures agents can comprise binding agents to one or more of: CD9, CD63, CD81, PSMA, PCSA, B7H3, EpCam, PSCA, ICAM, STEAP, and EGFR. In some embodiments, the plurality of capture agents comprise binding agents to CD9, CD63, CD81, PSMA, PCSA, B7H3, MFG-E8, and/or EpCam. In yet other embodiments, the plurality of capture agents comprises binding agents to CD9, CD63, CD81, PSMA, PCSA, B7H3, EpCam, PSCA, ICAM, STEAP, and/or EGFR. The plurality of capture agents comprises binding agents to TMEM211, MFG-E8, Tissue Factor (TF), and/or CD24.

The antibodies referenced herein can be immunoglobulin molecules or immunologically active portions of immunoglobulin molecules, i.e., molecules that contain an antigen binding site that specifically binds an antigen and synthetic antibodies. The immunoglobulin molecules can be of any class (e.g., IgG, IgE, IgM, IgD or IgA) or subclass of immunoglobulin molecule. Antibodies include, but are not limited to, polyclonal, monoclonal, bispecific, synthetic, humanized and chimeric antibodies, single chain antibodies, Fab fragments and F(ab′)₂ fragments, Fv or Fv′ portions, fragments produced by a Fab expression library, anti-idiotypic (anti-Id) antibodies, or epitope-binding fragments of any of the above. An antibody, or generally any molecule, “binds specifically” to an antigen (or other molecule) if the antibody binds preferentially to the antigen, and, e.g., has less than about 30%, 20%, 10%, 5% or 1% cross-reactivity with another molecule.

The binding agent can also be a polypeptide or peptide. Polypeptide is used in its broadest sense and may include a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the present invention may be chemically synthesized using standard techniques. The polypeptides may comprise D-amino acids (which are resistant to L-amino acid-specific proteases), a combination of D- and L-amino acids, amino acids, or various other designer or non-naturally occurring amino acids (e.g., β-methyl amino acids, Cα-methyl amino acids, and Nα-methyl amino acids, etc.) to convey special properties. Synthetic amino acids may include omithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the α-carbons, as in amino acids. Polypeptides and peptides are intended to be used interchangeably throughout this application, i.e. where the term peptide is used, it may also include polypeptides and where the term polypeptides is used, it may also include peptides. The term “protein” is also intended to be used interchangeably throughout this application with the terms “polypeptides” and “peptides” unless otherwise specified.

A vesicle may be isolated, captured or detected using a binding agent. The binding agent can be an agent that binds a vesicle “housekeeping protein,” or general vesicle biomarker. The biomarker can be CD63, CD9, CD81, CD82, CD37, CD53, Rab-5b, Annexin V or MFG-E8. Tetraspanins, a family of membrane proteins with four transmembrane domains, can be used as general vesicle markers. The tetraspanins include CD151, CD53, CD37, CD82, CD81, CD9 and CD63. There have been over 30 tetraspanins identified in mammals, including the TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (Oculospanin), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UPK1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN2? (CD82), TSPAN28 (CD81), TSPAN29 (CD9), TSPAN30 (CD63), TSPAN31 (SAS), TSPAN32 (TSSC6), TSPAN33, and TSPAN34. Other commonly observed vesicle markers include those listed in Table 3. Any of these proteins can be used as vesicle markers. Furthermore, any of the markers disclosed herein or in Table 3 can be selected in identifying a candidate biosignature for a disease or condition, where the one or more selected biomarkers have a direct or indirect role or function in mechanisms involved in the disease or condition.

TABLE 3
Proteins Observed in Vesicles from Multiple Cell Types

| Class | Protein |
|---|---|
| Antigen Presentation | MHC class I, MHC class II, Integrins, Alpha 4 beta 1, Alpha M beta 2, Beta 2 |
| Immunoglobulin family | ICAM1/CD54, P-selection |
| Cell-surface peptidases | Dipeptidylpeptidase IV/CD26, Aminopeptidase n/CD13 |
| Tetraspanins | TSPAN1 (TSP-1), TSPAN2 (TSP-2), TSPAN3 (TSP-3), TSPAN4 (TSP-4, |
| | NAG-2), TSPAN5 (TSP-5), TSPAN6 (TSP-6), TSPAN7 (CD231, TALLA-1, |
| | A15), TSPAN8 (CO-029), TSPAN9 (NET-5), TSPAN10 (Oculospanin), |
| | TSPAN11 (CD151-like), TSPAN12 (NET-2), TSPAN13 (NET-6), TSPAN14, |
| | TSPAN15 (NET-7), TSPAN16 (TM4-B), TSPAN17, TSPAN18, TSPAN19, |
| | TSPAN20 (UP1b, UPK1B), TSPAN21 (UP1a, UPK1A), TSPAN22 (RDS, |
| | PRPH2), TSPAN23 (ROM1), TSPAN24 (CD151), TSPAN25 (CD53), |
| | TSPAN26 (CD37), TSPAN27 (CD82), TSPAN28 (CD81), TSPAN29 (CD9), |
| | TSPAN30 (CD63), TSPAN31 (SAS), TSPAN32 (TSSC6), TSPAN33, and |
| | TSPAN34 |
| Heat-shock proteins | Hsp70, Hsp84/90 |
| Cytoskeletal proteins | Actin, Actin-binding proteins, Tubulin |
| Membrane transport and | Annexin I, Annexin II, Annexin IV, Annexin V, Annexin VI, |
| fusion | RAB7/RAP1B/RADGDI |
| Signal transduction | Gi2alpha/14-3-3, CBL/LCK |
| Abundant membrane | CD63, GAPDH, CD9, CD81, ANXA2, ENO1, SDCBP, MSN, MFGE8, EZR, |
| proteins | GK, ANXA1, LAMP2, DPP4, TSG101, HSPA1A, GDI2, CLTC, LAMP1, |
| | Cd86, ANPEP, TFRC, SLC3A2, RDX, RAP1B, RAB5C, RAB5B, MYH9, |
| | ICAM1, FN1, RAB11B, PIGR, LGALS3, ITGB1, EHD1, CLIC1, ATP1A1, |
| | ARF1, RAP1A, P4HB, MUC1, KRT10, HLA-A, FLOT1, CD59, C1orf58, |
| | BASP1, TACSTD1, STOM |
| Other Transmembrane | Cadherins: CDH1, CDH2, CDH12, CDH3, Deomoglein, DSG1, DSG2, DSG3, |
| Proteins | DSG4, Desmocollin, DSC1, DSC2, DSC3, Protocadherins, PCDH1, PCDH10, |
| | PCDH11x, PCDH11y, PCDH12, FAT, FAT2, FAT4, PCDH15, PCDH17, |
| | PCDH18, PCDH19; PCDH20; PCDH7, PCDH8, PCDH9, PCDHA1, |
| | PCDHA10, PCDHA11, PCDHA12, PCDHA13, PCDHA2, PCDHA3, |
| | PCDHA4, PCDHA5, PCDHA6, PCDHA7, PCDHA8, PCDHA9, PCDHAC1, |
| | PCDHAC2, PCDHB1, PCDHB10, PCDHB11, PCDHB12, PCDHB13, |
| | PCDHB14, PCDHB15, PCDHB16, PCDHB17, PCDHB18, PCDHB2, |
| | PCDHB3, PCDHB4, PCDHB5, PCDHB6, PCDHB7, PCDHB8, PCDHB9, |
| | PCDHGA1, PCDHGA10, PCDHGA11, PCDHGA12, PCDHGA2; PCDHGA3, |
| | PCDHGA4, PCDHGA5, PCDHGA6, PCDHGA7, PCDHGA8, PCDHGA9, |
| | PCDHGB1, PCDHGB2, PCDHGB3, PCDHGB4, PCDHGB5, PCDHGB6, |
| | PCDHGB7, PCDHGC3, PCDHGC4, PCDHGC5, CDH9 (cadherin 9, type 2 |
| | (T1-cadherin)), CDH10 (cadherin 10, type 2 (T2-cadherin)), CDH5 (VE- |
| | cadherin (vascular endothelial)), CDH6 (K-cadherin (kidney)), CDH7 (cadherin |
| | 7, type 2), CDH8 (cadherin 8, type 2), CDH11 (OB-cadherin (osteoblast)), |
| | CDH13 (T-cadherin-H-cadherin (heart)), CDH15 (M-cadherin (myotubule)), |
| | CDH16 (KSP-cadherin), CDH17 (LI cadherin (liver-intestine)), CDH18 |
| | (cadherin 18, type 2), CDH19 (cadherin 19, type 2), CDH20 (cadherin 20, type |
| | 2), CDH23 (cadherin 23, (neurosensory epithelium)), CDH10, CDH11, CDH13, |
| | CDH15, CDH16, CDH17, CDH18, CDH19, CDH20, CDH22, CDH23, CDH24, |
| | CDH26, CDH28, CDH4, CDH5, CDH6, CDH7, CDH8, CDH9, CELSR1, |
| | CELSR2, CELSR3, CLSTN1, CLSTN2, CLSTN3, DCHS1, DCHS2, |
| | LOC389118, PCLKC, RESDA1, RET |

The binding agent can also be an agent that binds to a vesicle derived from a specific cell type, such as a tumor cell (e.g. binding agent for Tissue factor, EpCam, B7H3, RAGE or CD24) or a specific cell-of-origin. The binding agent used to isolate or detect a vesicle can be a binding agent for an antigen selected from FIG. 1 of International Patent Application Serial No. PCT/US2011/031479, entitled “Circulating Biomarkers for Disease” and filed Apr. 6, 2011, which application is incorporated by reference in its entirety herein. The binding agent for a vesicle can also be selected from those listed in FIG. 2 of International Patent Application Serial No. PCT/US2011/031479. The binding agent can be for an antigen such as a tetraspanin, MFG-E8, Annexin V, 5T4, B7H3, caveolin, CD63, CD9, E-Cadherin, Tissue factor, MFG-E8, TMEM211, CD24, PSCA, PCSA, PSMA, Rab-5B, STEAP, TNFR1, CD81, EpCam, CD59, CD81, ICAM, EGFR, or CD66. A binding agent for a platelet can be a glycoprotein such as GpIa-IIa, GpIIb-IIIa, GpIIIb, GpIb, or GpIX. A binding agent can be for an antigen comprising one or more of CD9, Erb2, Erb4, CD81, Erb3, MUC16, CD63, DLL4, HLA-Drpe, B7H3, IFNAR, 5T4, PCSA, MICB, PSMA, MFG-E8, Muc1, PSA, Muc2, Unc93a, VEGFR2, EpCAM, VEGF A, TMPRSS2, RAGE, PSCA, CD40, Muc17, IL-17-RA, and CD80. For example, the binding agent can be one or more of CD9, CD63, CD81, B7H3, PCSA, MFG-E8, MUC2, EpCam, RAGE and Muc17. One or more binding agents, such as one or more binding agents for two or more of the antigens, can be used for isolating or detecting a vesicle. The binding agent used can be selected based on the desire of isolating or detecting a vesicle derived from a particular cell type or cell-of-origin specific vesicle.

A binding agent can also be linked directly or indirectly to a solid surface or substrate. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, polytetrafluoroethylene (PTFE, Teflon®), etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

For example, an antibody used to isolate a vesicle can be bound to a solid substrate such as a well, such as commercially available plates (e.g. from Nunc, Milan Italy). Each well can be coated with the antibody. In some embodiments, the antibody used to isolate a vesicle is bound to a solid substrate such as an array. The array can have a predetermined spatial arrangement of molecule interactions, binding islands, biomolecules, zones, domains or spatial arrangements of binding islands or binding agents deposited within discrete boundaries. Further, the term array may be used herein to refer to multiple arrays arranged on a surface, such as would be the case where a surface bore multiple copies of an array. Such surfaces bearing multiple arrays may also be referred to as multiple arrays or repeating arrays.

Arrays typically contain addressable moieties that can detect the presense of an entity, e.g., a vesicle in the sample via a binding event. An array may be referred to as a microarray. Arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein—protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. In a non-limiting example, a protein array can be used to detect vesicles having certain proteins on their surface. Antibody arrays comprise antibodies spotted onto the protein chip that are used as capture molecules to detect proteins or other biological materials from a sample, e.g., from cell or tissue lysate solutions. For example, antibody arrays can be used to detect vesicle-associated biomarkers from bodily fluids, e.g., serum or urine. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Cellular arrays can also be used to capture vesicles due to the similarity between a vesicle and cellular membrane. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

A binding agent can also be bound to particles such as beads or microspheres. For example, an antibody specific for a component of a vesicle can be bound to a particle, and the antibody-bound particle is used to isolate a vesicle from a biological sample. In some embodiments, the microspheres may be magnetic or fluorescently labeled. In addition, a binding agent for isolating vesicles can be a solid substrate itself. For example, latex beads, such as aldehyde/sulfate beads (Interfacial Dynamics, Portland, Oreg.) can be used.

A binding agent bound to a magnetic bead can also be used to isolate a vesicle. For example, a biological sample such as serum from a patient can be collected for colon cancer screening. The sample can be incubated with anti-CCSA-3 (Colon Cancer-Specific Antigen) coupled to magnetic microbeads. A low-density microcolumn can be placed in the magnetic field of a MACS Separator and the column is then washed with a buffer solution such as Tris-buffered saline. The magnetic immune complexes can then be applied to the column and unbound, non-specific material can be discarded. The CCSA-3 selected vesicle can be recovered by removing the column from the separator and placing it on a collection tube. A buffer can be added to the column and the magnetically labeled vesicle can be released by applying the plunger supplied with the column. The isolated vesicle can be diluted in IgG elution buffer and the complex can then be centrifuged to separate the microbeads from the vesicle. The pelleted isolated cell-of-origin specific vesicle can be resuspended in buffer such as phosphate-buffered saline and quantitated. Alternatively, due to the strong adhesion force between the antibody captured cell-of-origin specific vesicle and the magnetic microbeads, a proteolytic enzyme such as trypsin can be used for the release of captured vesicles without the need for centrifugation. The proteolytic enzyme can be incubated with the antibody captured cell-of-origin specific vesicles for at least a time sufficient to release the vesicles.

A binding agent, such as an antibody, for isolating vesicles is preferably contacted with the biological sample comprising the vesicles of interest for at least a time sufficient for the binding agent to bind to a component of the vesicle. For example, an antibody may be contacted with a biological sample for various intervals ranging from seconds days, including but not limited to, about 10 minutes, 30 minutes, 1 hour, 3 hours, 5 hours, 7 hours, 10 hours, 15 hours, 1 day, 3 days, 7 days or 10 days.

A binding agent, such as an antibody specific to an antigen listed in FIG. 1 of International Patent Application Serial No. PCT/US2011/031479, entitled “Circulating Biomarkers for Disease” and filed Apr. 6, 2011, which application is incorporated by reference in its entirety herein, or a binding agent listed in FIG. 2 of International Patent Application Serial No. PCT/US2011/031479, can be labeled to facilitate detection. Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as 3H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

A binding agent can be directly or indirectly labeled, e.g., the label is attached to the antibody through biotin-streptavidin. Alternatively, an antibody is not labeled, but is later contacted with a second antibody that is labeled after the first antibody is bound to an antigen of interest.

For example, various enzyme-substrate labels are available or disclosed (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), (3-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (f3-D-Gal) with a chromogenic substrate (e.g., p-nitrophenyl-β-D-galactosidase) or fluorogenic substrate 4-methylumbelliferyl-β-D-galactosidase.

Depending on the method of isolation or detection used, the binding agent may be linked to a solid surface or substrate, such as arrays, particles, wells and other substrates described above. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

### Particle-Based Assays

As an alternative to planar arrays, assays using particles or microspheres, such as bead based assays, are capable of use with a binding agent. For example, antibodies or aptamers are easily conjugated with commercially available beads. See, e.g., Fan et al., Illumina universal bead arrays. Methods Enzymol. 2006 410:57-73; Srinivas et al. Anal. Chem. 2011 Oct. 21, Aptamer functionalized Microgel Particles for Protein Detection; See also, review article on aptamers as therapeutic and diagnostic agents, Brody and Gold, Rev. Mol. Biotech. 2000, 74:5-13.

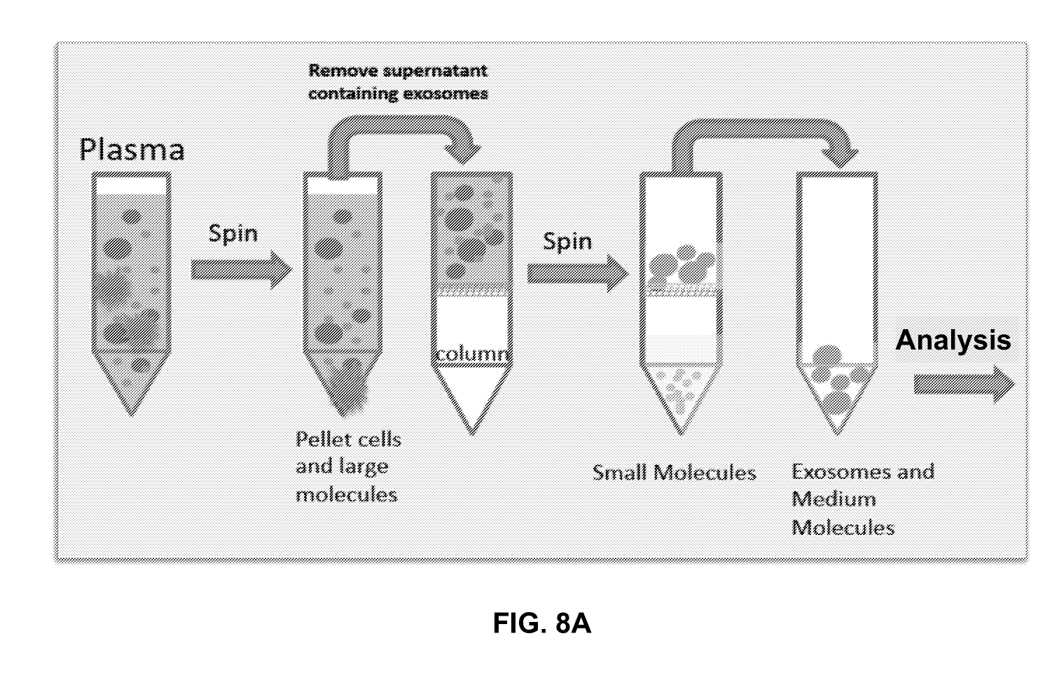

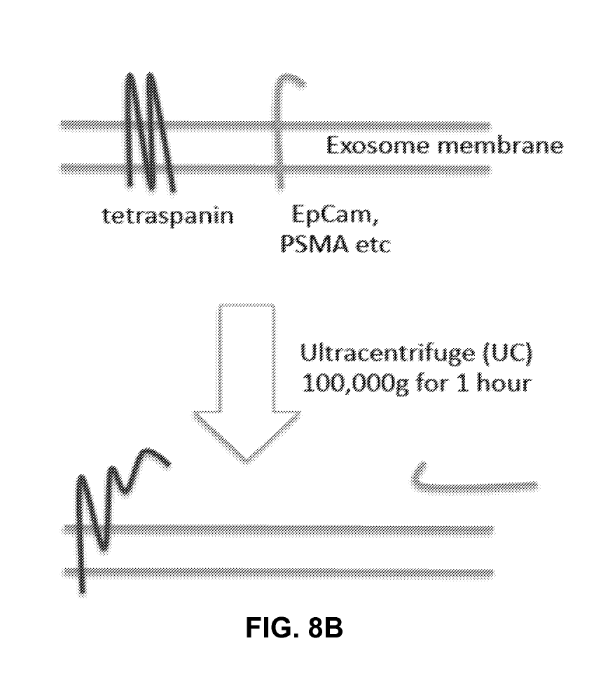

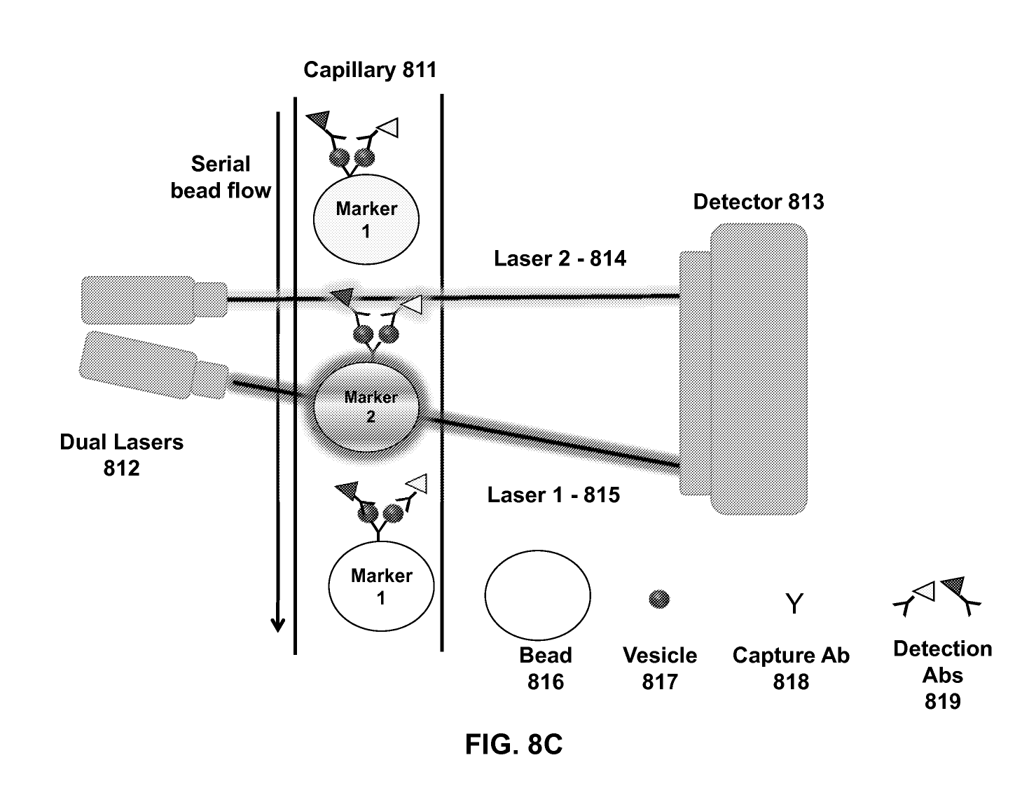

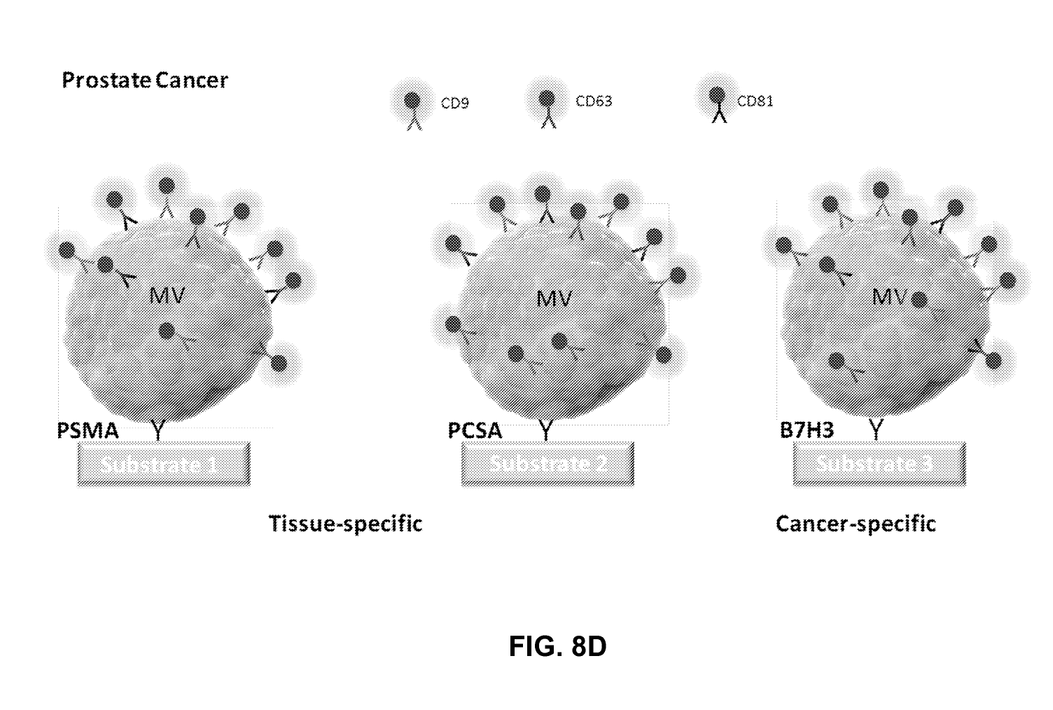

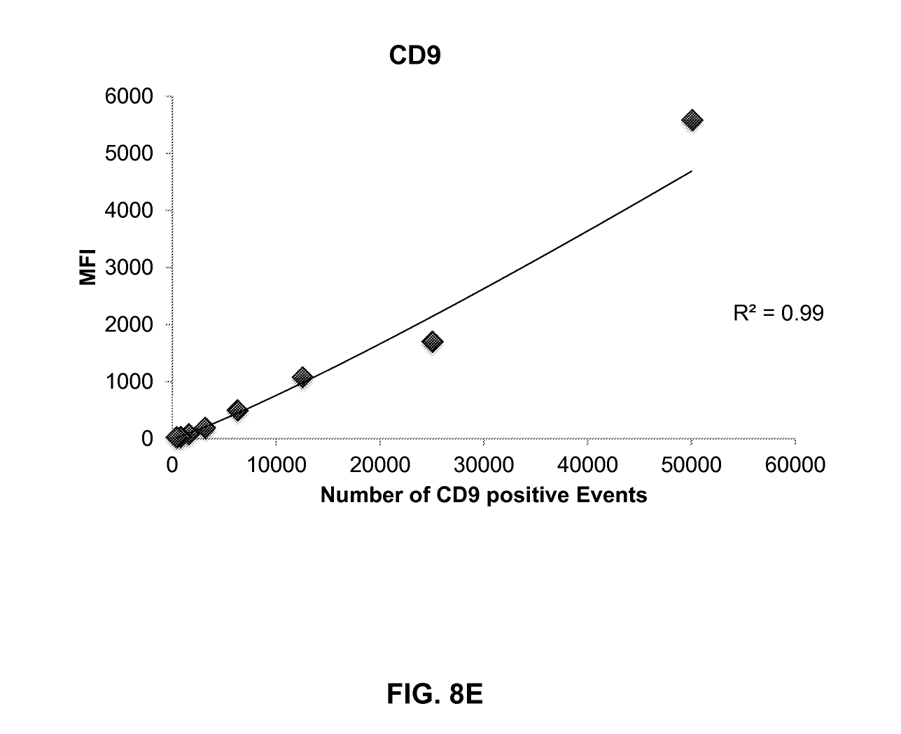

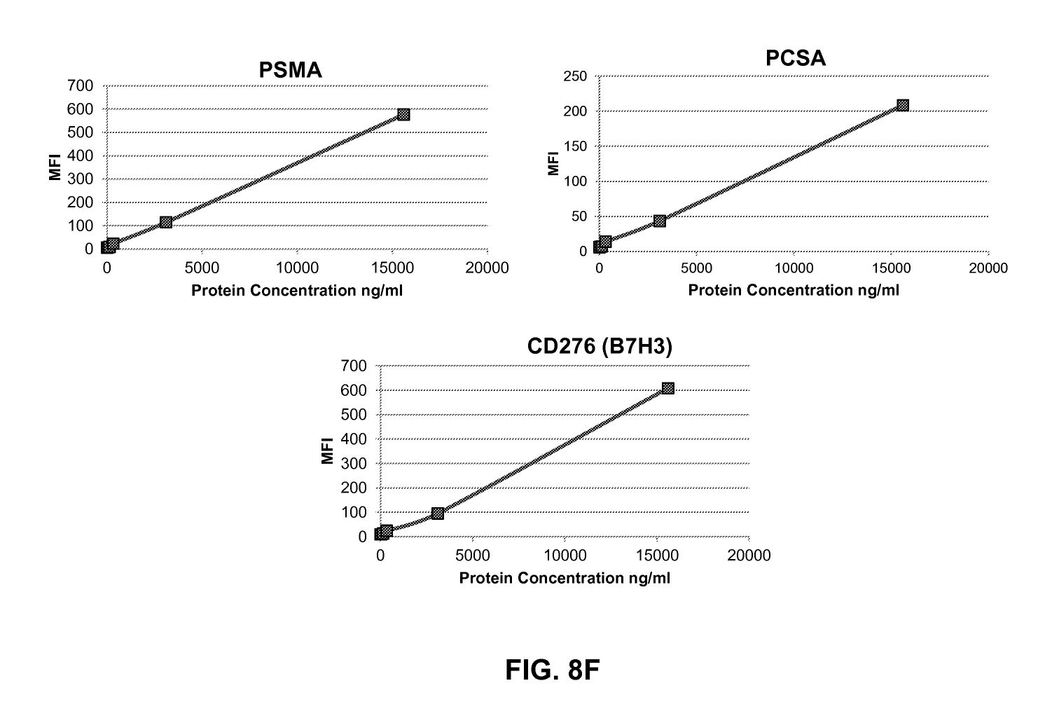

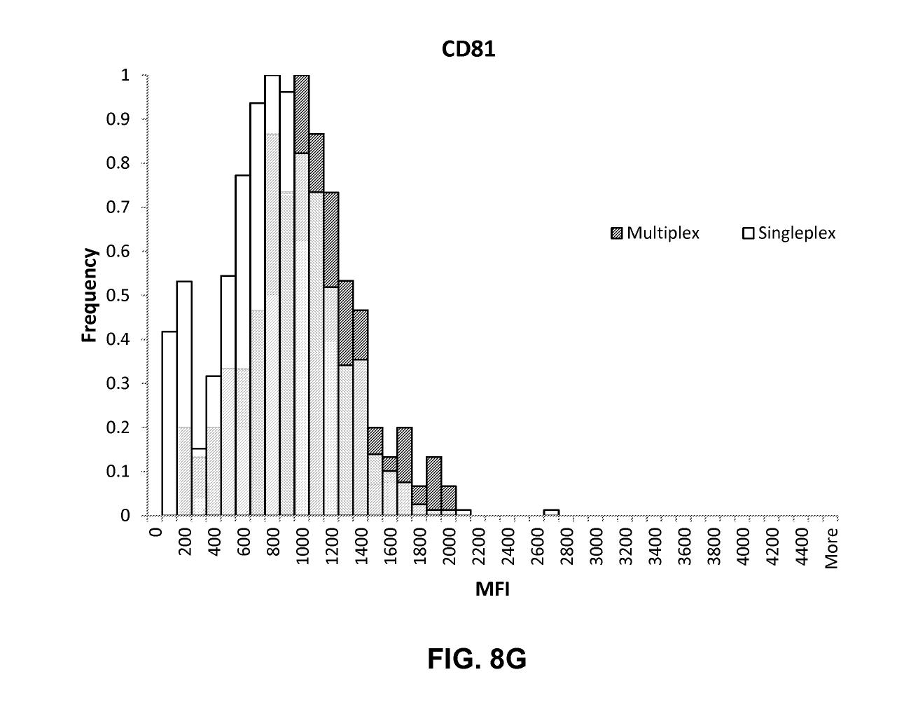

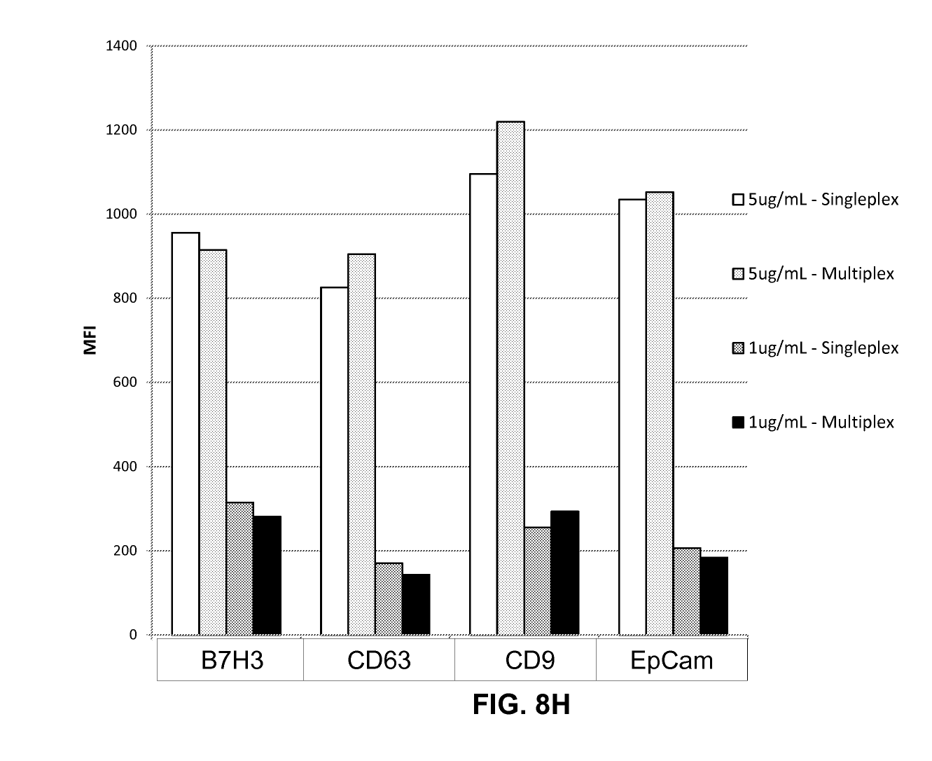

Multiparametric assays or other high throughput detection assays using bead coatings with cognate ligands and reporter molecules with specific activities consistent with high sensitivity automation can be used. In a bead based assay system, a binding agent for a biomarker or vesicle, such as a capture agent (e.g. capture antibody), can be immobilized on an addressable microsphere. Each binding agent for each individual binding assay can be coupled to a distinct type of microsphere (i.e., microbead) and the assay reaction takes place on the surface of the microsphere, such as depicted in FIG. 2B. A binding agent for a vesicle can be a capture antibody or aptamer coupled to a bead. Dyed microspheres with discrete fluorescence intensities are loaded separately with their appropriate binding agent or capture probes. The different bead sets carrying different binding agents can be pooled as necessary to generate custom bead arrays. Bead arrays are then incubated with the sample in a single reaction vessel to perform the assay. See FIGS. 8C-D for illustrative methods of detecting microvesicles using microbeads with antibody binding agents.

A bead substrate can provide a platform for attaching one or more binding agents, including aptamer(s) or antibodies. One of skill will appreciate that the illustrative schemes shown in FIGS. 8C-D can employ aptamers along with or instead of antibodies. For multiplexing, multiple different bead sets (e.g., those commercially available from Illumina, Inc., San Diego, Calif., USA, or Luminex Corporation, Austin, Tex., USA) can have different binding agents which are specific to different target molecules. Beads can also be used for different purposes, e.g., detection and/or isolation. For example, a bead can be conjugated to an aptamer used to detect the presence (quantitatively or qualitatively) of a given biomarker, or it can also be used to isolate a component present in a selected biological sample (e.g., cell, cell-fragment or vesicle comprising the target molecule to which the binding agent is configured to bind or associate). Various molecules of organic origin can be conjugated to a microbeads, e.g., polysterene beads, through use of commercially available kits. One of skill will appreciate that an assay can use multiple types of binding agents. For example, a bead may be conjugated to an aptamer which serves to bind and capture a biomarker, and a labeled antibody can be used to further detect the captured biomarker. Similarly, a bead may be conjugated to an antibody which serves to bind and capture a biomarker, and a labeled aptamer can be used to further detect the captured biomarker. Any such useful combination of binding agents are contemplated by the invention.

Bead-based assays can also be used with one or more binding agents such as antibodies or aptamers. A bead substrate can provide a platform for attaching the one or more binding agents. For multiplexing, multiple different bead sets (e.g., as provided by Illumina or Luminex) can have different binding agents (specific to different target molecules). For example, a bead can be conjugated to a binding agent, e.g., an aptamer of the invention, used to detect the presence (quantitatively or qualitatively) of an antigen of interest, or it can also be used to isolate a component present in a selected biological sample (e.g., cell, cell-fragment or vesicle comprising the target molecule to which the aptamer is configured to bind or associate). Any molecule of organic origin can be successfully conjugated to a polystyrene bead through use of commercially available kits.

One or more binding agent can be used with any bead based substrate, including but not limited to magnetic capture method, fluorescence activated cell sorting (FACS) or laser cytometry. Magnetic capture methods can include, but are not limited to, the use of magnetically activated cell sorter (MACS) microbeads or magnetic columns. Examples of bead or particle based methods that can be used in the methods of the invention include the bead systems described in any of U.S. Pat. Nos. 4,551,435, 4,795,698, 4,925,788, 5,108,933, 5,186,827, 5,200,084 or 5,158,871; 7,399,632; 8,124,015; 8,008,019; 7,955,802; 7,445,844; 7,274,316; 6,773,812; 6,623,526; 6,599,331; 6,057,107; 5,736,330; or International Patent Application Nos. PCT/US2012/42519; PCT/US1993/04145.

### Flow Cytometry

Isolation or detection of circulating biomarkers, e.g., protein antigens, from a biological sample, or of the biomarker-comprising cells, cell fragments or vesicles may also be achieved using a cytometry process. As a non-limiting example, aptamers or antibodies can be used in an assay comprising using a particle such as a bead or microsphere. Flow cytometry can be used for sorting microscopic particles suspended in a stream of fluid. As particles pass through they can be selectively charged and on their exit can be deflected into separate paths of flow. It is therefore possible to separate populations from an original mix, such as a biological sample, with a high degree of accuracy and speed. Flow cytometry allows simultaneous multiparametric analysis of the physical and/or chemical characteristics of single cells flowing through an optical/electronic detection apparatus. A beam of light, usually laser light, of a single frequency (color) is directed onto a hydrodynamically focused stream of fluid. A number of detectors are aimed at the point where the stream passes through the light beam; one in line with the light beam (Forward Scatter or FSC) and several perpendicular to it (Side Scatter or SSC) and one or more fluorescent detectors.

Each suspended particle passing through the beam scatters the light in some way, and fluorescent chemicals in the particle may be excited into emitting light at a lower frequency than the light source. This combination of scattered and fluorescent light is picked up by the detectors, and by analyzing fluctuations in brightness at each detector (one for each fluorescent emission peak), it is possible to deduce various facts about the physical and chemical structure of each individual particle. FSC correlates with the cell size and SSC depends on the inner complexity of the particle, such as shape of the nucleus, the amount and type of cytoplasmic granules or the membrane roughness. Some flow cytometers have eliminated the need for fluorescence and use only light scatter for measurement.

Flow cytometers can analyze several thousand particles every second in “real time” and can actively separate out and isolate particles having specified properties. They offer high-throughput automated quantification, and separation, of the set parameters for a high number of single cells during each analysis session. Flow cytomers can have multiple lasers and fluorescence detectors, allowing multiple labels to be used to more precisely specify a target population by their phenotype. Thus, a flow cytometer, such as a multicolor flow cytometer, can be used to detect one or more vesicles with multiple fluorescent labels or colors. In some embodiments, the flow cytometer can also sort or isolate different vesicle populations, such as by size or by different markers.

The flow cytometer may have one or more lasers, such as 1, 2, 3, 4, 5, 6, 7, 8, 9, 10 or more lasers. In some embodiments, the flow cytometer can detect more than one color or fluorescent label, such as at least 2, 3, 4, 5, 6, 7, 8, 9, 10, 11, 12, 13, 14, 15, 16, 17, 18, 19, or 20 different colors or fluorescent labels. For example, the flow cytometer can have at least 2, 3, 4, 5, 6, 7, 8, 9, 10, 11, 12, 13, 14, 15, 16, 17, 18, 19, or 20 fluorescence detectors.

Examples of commercially available flow cytometers that can be used to detect or analyze one or more vesicles, to sort or separate different populations of vesicles, include, but are not limited to the MoFlo™ XDP Cell Sorter (Beckman Coulter, Brea, Calif.), MoFlo™ Legacy Cell Sorter (Beckman Coulter, Brea, Calif.), BD FACSAria™ Cell Sorter (BD Biosciences, San Jose, Calif.), BD™ LSRII (BD Biosciences, San Jose, Calif.), and BD FACSCalibur™ (BD Biosciences, San Jose, Calif.). Use of multicolor or multi-fluor cytometers can be used in multiplex analysis of vesicles, as further described below. In some embodiments, the flow cytometer can sort, and thereby collect or sort more than one population of vesicles based one or more characteristics. For example, two populations of vesicles differ in size, such that the vesicles within each population have a similar size range and can be differentially detected or sorted. In another embodiment, two different populations of vesicles are differentially labeled.

The data resulting from flow-cytometers can be plotted in 1 dimension to produce histograms or seen in 2 dimensions as dot plots or in 3 dimensions with newer software. The regions on these plots can be sequentially separated by a series of subset extractions which are termed gates. Specific gating protocols exist for diagnostic and clinical purposes especially in relation to hematology. The plots are often made on logarithmic scales. Because different fluorescent dye's emission spectra overlap, signals at the detectors have to be compensated electronically as well as computationally. Fluorophores for labeling biomarkers may include those described in Ormerod, Flow Cytometry 2nd ed., Springer-Verlag, New York (1999), and in Nida et al., Gynecologic Oncology 2005; 4 889-894 which is incorporated herein by reference. In a multiplexed assay, including but not limited to a flow cytometry assay, one or more different target molecules can be assessed. In some embodiments, at least one of the target molecule is a biomarker, e.g., a microvesicle surface antigen.

In various embodiments of the invention, flow cytometry is used to assess a microvesicle population in a biological sample. If desired, the microvesicle population can be sorted from other particles (e.g., cell debris, protein aggregates, etc) in a sample by labeling the vesicles using one or more general vesicle marker. The general vesicle marker can be a marker in Table 3. Commonly used vesicle markers include tetraspanins such as CD9, CD63 and/or CD81. Vesicles comprising one or more tetraspanin are sometimes refered to as “Tet+” herein to indicate that the vesicles are tetraspanin-positive. The sorted microvesicles can be further assessed using methodology described herein. E.g., surface antigens on the sorted microvesicles can be detected using flow or other methods. In some embodiments, payload within the sorted microvesicles is assessed. As an illustrative example, a population of microvesicles is contacted with a labeled binding agent to a surface antigen of interest, the contacted microvesicles are sorted using flow cytometry, and payload with the microvesicles is assessed. The payload may be polypeptides, nucleic acids (e.g., mRNA or microRNA) or other biological entities as desired. Such assessment is used to characterize a phenotype as described herein, e.g., to diagnose, prognose or theranose a cancer.

In an embodiment, flow sorting is used to distinguish microvesicle populations from other biological complexes. In a non-limiting example, Ago2+/Tet+ and Ago2+/Tet-particles are detected using flow methodology to separate Ago2+ vesicles from vesicle-free Ago2+ complexes, respectively.

### Multiplexing

Multiplex experiments comprise experiments that can simultaneously measure multiple analytes in a single assay. Vesicles and associated biomarkers can be assessed in a multiplex fashion. Different binding agents can be used for multiplexing different circulating biomarkers, e.g., microRNA, protein, or vesicle populations. Different biomarkers, e.g., different vesicle populations, can be isolated or detected using different binding agents. Each population in a biological sample can be labeled with a different signaling label, such as a fluorophore, quantum dot, or radioactive label, such as described above. The label can be directly conjugated to a binding agent or indirectly used to detect a binding agent that binds a vesicle. The number of populations detected in a multiplexing assay is dependent on the resolution capability of the labels and the summation of signals, as more than two differentially labeled vesicle populations that bind two or more affinity elements can produce summed signals.

Multiplexing of at least 2, 3, 4, 5, 6, 7, 8, 9, 10, 11, 12, 13, 14, 15, 16, 17, 18, 19, 20, 25, 50, 75 or 100 different circulating biomarkers may be performed. For example, one population of vesicles specific to a cell-of-origin can be assayed along with a second population of vesicles specific to a different cell-of-origin, where each population is labeled with a different label. Alternatively, a population of vesicles with a particular biomarker or biosignature can be assayed along with a second population of vesicles with a different biomarker or biosignature. In some cases, hundreds or thousands of vesicles are assessed in a single assay.

In one embodiment, multiplex analysis is performed by applying a plurality of vesicles comprising more than one population of vesicles to a plurality of substrates, such as beads. Each bead is coupled to one or more capture agents. The plurality of beads is divided into subsets, where beads with the same capture agent or combination of capture agents form a subset of beads, such that each subset of beads has a different capture agent or combination of capture agents than another subset of beads. The beads can then be used to capture vesicles that comprise a component that binds to the capture agent. The different subsets can be used to capture different populations of vesicles. The captured vesicles can then be analyzed by detecting one or more biomarkers.

Flow cytometry can be used in combination with a particle-based or bead based assay. Multiparametric immunoassays or other high throughput detection assays using bead coatings with cognate ligands and reporter molecules with specific activities consistent with high sensitivity automation can be used. For example, beads in each subset can be differentially labeled from another subset. In a particle based assay system, a binding agent or capture agent for a vesicle, such as a capture antibody, can be immobilized on addressable beads or microspheres. Each binding agent for each individual binding assay (such as an immunoassay when the binding agent is an antibody) can be coupled to a distinct type of microsphere (i.e., microbead) and the binding assay reaction takes place on the surface of the microspheres. Microspheres can be distinguished by different labels, for example, a microsphere with a specific capture agent would have a different signaling label as compared to another microsphere with a different capture agent. For example, microspheres can be dyed with discrete fluorescence intensities such that the fluorescence intensity of a microsphere with a specific binding agent is different than that of another microsphere with a different binding agent. Biomarkers bound by different capture agents can be differentially detected using different labels.

A microsphere can be labeled or dyed with at least 2 different labels or dyes. In some embodiments, the microsphere is labeled with at least 3, 4, 5, 6, 7, 8, 9, or 10 different labels. Different microspheres in a plurality of microspheres can have more than one label or dye, wherein various subsets of the microspheres have various ratios and combinations of the labels or dyes permitting detection of different microspheres with different binding agents. For example, the various ratios and combinations of labels and dyes can permit different fluorescent intensities. Alternatively, the various ratios and combinations maybe used to generate different detection patters to identify the binding agent. The microspheres can be labeled or dyed externally or may have intrinsic fluorescence or signaling labels. Beads can be loaded separately with their appropriate binding agents and thus, different vesicle populations can be isolated based on the different binding agents on the differentially labeled microspheres to which the different binding agents are coupled.

In another embodiment, multiplex analysis can be performed using a planar substrate, wherein the substrate comprises a plurality of capture agents. The plurality of capture agents can capture one or more populations of vesicles, and one or more biomarkers of the captured vesicles detected. The planar substrate can be a microarray or other substrate as further described herein.

### Binding Agents

A vesicle may be isolated or detected using a binding agent for a novel component of a vesicle, such as an antibody for a novel antigen specific to a vesicle of interest. Novel antigens that are specific to a vesicle of interest may be isolated or identified using different test compounds of known composition bound to a substrate, such as an array or a plurality of particles, which can allow a large amount of chemical/structural space to be adequately sampled using only a small fraction of the space. The novel antigen identified can also serve as a biomarker for the vesicle. For example, a novel antigen identified for a cell-of-origin specific vesicle can be a useful biomarker.

The term “agent” or “reagent” as used in respect to contacting a sample can mean any entity designed to bind, hybridize, associate with or otherwise detect or facilitate detection of a target molecule, including target polypeptides, peptides, nucleic acid molecules, leptins, lipids, or any other biological entity that can be detected as described herein or as known in the art. Examples of such agents/reagents are well known in the art, and include but are not limited to universal or specific nucleic acid primers, nucleic acid probes, antibodies, aptamers, peptoid, peptide nucleic acid, locked nucleic acid, lectin, dendrimer, chemical compound, or other entities described herein or known in the art.

A binding agent can be identified by screening either a homogeneous or heterogeneous vesicle population against test compounds. Since the composition of each test compound on the substrate surface is known, this constitutes a screen for affinity elements. For example, a test compound array comprises test compounds at specific locations on the substrate addressable locations, and can be used to identify one or more binding agents for a vesicle. The test compounds can all be unrelated or related based on minor variations of a core sequence or structure. The different test compounds may include variants of a given test compound (such as polypeptide isoforms), test compounds that are structurally or compositionally unrelated, or a combination thereof.

A test compound can be a peptoid, polysaccharide, organic compound, inorganic compound, polymer, lipids, nucleic acid, polypeptide, antibody, protein, polysaccharide, or other compound. The test compound can be natural or synthetic. The test compound can comprise or consist of linear or branched heteropolymeric compounds based on any of a number of linkages or combinations of linkages (e.g., amide, ester, ether, thiol, radical additions, metal coordination, etc.), dendritic structures, circular structures, cavity structures or other structures with multiple nearby sites of attachment that serve as scaffolds upon which specific additions are made. Thes test compound can be spotted on a substrate or synthesized in situ, using standard methods in the art. In addition, the test compound can be spotted or synthesized in situ in combinations in order to detect useful interactions, such as cooperative binding.

The test compound can be a polypeptide with known amino acid sequence, thus, detection of a test compound binding with a vesicle can lead to identification of a polypeptide of known amino sequence that can be used as a binding agent. For example, a homogenous population of vesicles can be applied to a spotted array on a slide containing between a few and 1,000,000 test polypeptides having a length of variable amino acids. The polypeptides can be attached to the surface through the C-terminus. The sequence of the polypeptides can be generated randomly from 19 amino acids, excluding cysteine. The binding reaction can include a non-specific competitor, such as excess bacterial proteins labeled with another dye such that the specificity ratio for each polypeptide binding target can be determined. The polypeptides with the highest specificity and binding can be selected. The identity of the polypeptide on each spot is known, and thus can be readily identified. Once the novel antigens specific to the homogeneous vesicle population, such as a cell-of-origin specific vesicle is identified, such cell-of-origin specific vesicles may subsequently be isolated using such antigens in methods described hereafter.

An array can also be used for identifying an antibody as a binding agent for a vesicle. Test antibodies can be attached to an array and screened against a heterogeneous population of vesicles to identify antibodies that can be used to isolate or identify a vesicle. A homogeneous population of vesicles such as cell-of-origin specific vesicles can also be screened with an antibody array. Other than identifying antibodies to isolate or detect a homogeneous population of vesicles, one or more protein biomarkers specific to the homogenous population can be identified. Commercially available platforms with test antibodies pre-selected or custom selection of test antibodies attached to the array can be used. For example, an antibody array from Full Moon Biosystems can be screened using prostate cancer cell derived vesicles identifying antibodies to Bcl-XL, ERCC1, Keratin 15, CD81/TAPA-1, CD9, Epithelial Specific Antigen (ESA), and Mast Cell Chymase as binding agents, and the proteins identified can be used as biomarkers for the vesicles. The biomarker can be present or absent, underexpressed or overexpressed, mutated, or modified in or on a vesicle and used in characterizing a condition.

An antibody or synthetic antibody to be used as a binding agent can also be identified through a peptide array. Another method is the use of synthetic antibody generation through antibody phage display. M13 bacteriophage libraries of antibodies (e.g. Fabs) are displayed on the surfaces of phage particles as fusions to a coat protein. Each phage particle displays a unique antibody and also encapsulates a vector that contains the encoding DNA. Highly diverse libraries can be constructed and represented as phage pools, which can be used in antibody selection for binding to immobilized antigens. Antigen-binding phages are retained by the immobilized antigen, and the nonbinding phages are removed by washing. The retained phage pool can be amplified by infection of an Escherichia coli host and the amplified pool can be used for additional rounds of selection to eventually obtain a population that is dominated by antigen-binding clones. At this stage, individual phase clones can be isolated and subjected to DNA sequencing to decode the sequences of the displayed antibodies. Through the use of phase display and other methods known in the art, high affinity designer antibodies for vesicles can be generated.

Bead-based assays can also be used to identify novel binding agents to isolate or detect a vesicle. A test antibody or peptide can be conjugated to a particle. For example, a bead can be conjugated to an antibody or peptide and used to detect and quantify the proteins expressed on the surface of a population of vesicles in order to discover and specifically select for novel antibodies that can target vesicles from specific tissue or tumor types. Any molecule of organic origin can be successfully conjugated to a polystyrene bead through use of a commercially available kit according to manufacturer's instructions. Each bead set can be colored a certain detectable wavelength and each can be linked to a known antibody or peptide which can be used to specifically measure which beads are linked to exosomal proteins matching the epitope of previously conjugated antibodies or peptides. The beads can be dyed with discrete fluorescence intensities such that each bead with a different intensity has a different binding agent as described above.

For example, a purified vesicle preparation can be diluted in assay buffer to an appropriate concentration according to empirically determined dynamic range of assay. A sufficient volume of coupled beads can be prepared and approximately 1 μl of the antibody-coupled beads can be aliqouted into a well and adjusted to a final volume of approximately 50 Once the antibody-conjugated beads have been added to a vacuum compatible plate, the beads can be washed to ensure proper binding conditions. An appropriate volume of vesicle preparation can then be added to each well being tested and the mixture incubated, such as for 15-18 hours. A sufficient volume of detection antibodies using detection antibody diluent solution can be prepared and incubated with the mixture for 1 hour or for as long as necessary. The beads can then be washed before the addition of detection antibody (biotin expressing) mixture composed of streptavidin phycoereythin. The beads can then be washed and vacuum aspirated several times before analysis on a suspension array system using software provided with an instrument. The identity of antigens that can be used to selectively extract the vesicles can then be elucidated from the analysis.

Assays using imaging systems can be used to detect and quantify proteins expressed on the surface of a vesicle in order to discover and specifically select for and enrich vesicles from specific tissue, cell or tumor types. Antibodies, peptides or cells conjugated to multiple well multiplex carbon coated plates can be used. Simultaneous measurement of many analytes in a well can be achieved through the use of capture antibodies arrayed on the patterned carbon working surface. Analytes can then be detected with antibodies labeled with reagents in electrode wells with an enhanced electro-chemiluminescent plate. Any molecule of organic origin can be successfully conjugated to the carbon coated plate. Proteins expressed on the surface of vesicles can be identified from this assay and can be used as targets to specifically select for and enrich vesicles from specific tissue or tumor types.

The binding agent can also be an aptamer, which refers to nucleic acids that can bond molecules other than their complementary sequence. An aptamer typically contains 30-80 nucleic acids and can have a high affinity towards a certain target molecule (K_{d}'s reported are between 10⁻¹¹-10⁻⁶mole/1). An aptamer for a target can be identified using systematic evolution of ligands by exponential enrichment (SELEX) (Tuerk & Gold, Science 249:505-510, 1990; Ellington & Szostak, Nature 346:818-822, 1990), such as described in U.S. Pat. Nos. 5,270,163, 6,482, 594, 6,291, 184, 6,376, 190 and U.S. Pat. No. 6,458,539. A library of nucleic acids can be contacted with a target vesicle, and those nucleic acids specifically bound to the target are partitioned from the remainder of nucleic acids in the library which do not specifically bind the target. The partitioned nucleic acids are amplified to yield a ligand-enriched pool. Multiple cycles of binding, partitioning, and amplifying (i.e., selection) result in identification of one or more aptamers with the desired activity. Another method for identifying an aptamer to isolate vesicles is described in U.S. Pat. No. 6,376,190, which describes increasing or decreasing frequency of nucleic acids in a library by their binding to a chemically synthesized peptide. Modified methods, such as Laser SELEX or deSELEX as described in U.S. Patent Publication No. 20090264508 can also be used.

The term “specific” as used herein in regards to a binding agent can mean that an agent has a greater affinity for its target than other targets, typically with a much great affinity, but does not require that the binding agent is absolutely specific for its target.

### Microfluidics

The methods for isolating or identifying vesicles can be used in combination with microfluidic devices. The methods of isolating or detecting a vesicle, such as described herien, can be performed using a microfluidic device. Microfluidic devices, which may also be referred to as “lab-on-a-chip” systems, biomedical micro-electro-mechanical systems (bioMEMs), or multicomponent integrated systems, can be used for isolating and analyzing a vesicle. Such systems miniaturize and compartmentalize processes that allow for binding of vesicles, detection of biosignatures, and other processes.

A microfluidic device can also be used for isolation of a vesicle through size differential or affinity selection. For example, a microfluidic device can use one more channels for isolating a vesicle from a biological sample based on size or by using one or more binding agents for isolating a vesicle from a biological sample. A biological sample can be introduced into one or more microfluidic channels, which selectively allows the passage of a vesicle. The selection can be based on a property of the vesicle, such as the size, shape, deformability, or biosignature of the vesicle.

In one embodiment, a heterogeneous population of vesicles can be introduced into a microfluidic device, and one or more different homogeneous populations of vesicles can be obtained. For example, different channels can have different size selections or binding agents to select for different vesicle populations. Thus, a microfluidic device can isolate a plurality of vesicles wherein at least a subset of the plurality of vesicles comprises a different biosignature from another subset of the plurality of vesicles. For example, the microfluidic device can isolate at least 2, 3, 4, 5, 6, 7, 8, 9, 10, 15, 20, 25, 30, 40, 50, 60, 70, 80, 90, or 100 different subsets of vesicles, wherein each subset of vesicles comprises a different biosignature.

In some embodiments, the microfluidic device can comprise one or more channels that permit further enrichment or selection of a vesicle. A population of vesicles that has been enriched after passage through a first channel can be introduced into a second channel, which allows the passage of the desired vesicle or vesicle population to be further enriched, such as through one or more binding agents present in the second channel.

Array-based assays and bead-based assays can be used with microfluidic device. For example, the binding agent can be coupled to beads and the binding reaction between the beads and vesicle can be performed in a microfluidic device. Multiplexing can also be performed using a microfluidic device. Different compartments can comprise different binding agents for different populations of vesicles, where each population is of a different cell-of-origin specific vesicle population. In one embodiment, each population has a different biosignature. The hybridization reaction between the microsphere and vesicle can be performed in a microfluidic device and the reaction mixture can be delivered to a detection device. The detection device, such as a dual or multiple laser detection system can be part of the microfluidic system and can use a laser to identify each bead or microsphere by its color-coding, and another laser can detect the hybridization signal associated with each bead.

Any appropriate microfluidic device can be used in the methods of the invention. Examples of microfluidic devices that may be used, or adapted for use with vesicles, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, 7,118,661, 7,640,947, 7,666,361, 7,704,735; and International Patent Publication WO 2010/072410; each of which patents or applications are incorporated herein by reference in their entirety. Another example for use with methods disclosed herein is described in Chen et al., “Microfluidic isolation and transcriptome analysis of serum vesicles,” Lab on a Chip, Dec. 8, 2009 DOI: 10.1039/b916199f.

Other microfluidic devices for use with the invention include devices comprising elastomeric layers, valves and pumps, including without limitation those disclosed in U.S. Pat. Nos. 5,376,252, 6,408,878, 6,645,432, 6,719,868, 6,793,753, 6,899,137, 6,929,030, 7,040,338, 7,118,910, 7,144,616, 7,216,671, 7,250,128, 7,494,555, 7,501,245, 7,601,270, 7,691,333, 7,754,010, 7,837,946; U.S. Patent Application Nos. 2003/0061687, 2005/0084421, 2005/0112882, 2005/0129581, 2005/0145496, 2005/0201901, 2005/0214173, 2005/0252773, 2006/0006067; and EP Patent Nos. 0527905 and 1065378; each of which application is herein incorporated by reference. In some instances, much or all of the devices are composed of elastomeric material. Certain devices are designed to conduct thermal cycling reactions (e.g., PCR) with devices that include one or more elastomeric valves to regulate solution flow through the device. The devices can comprise arrays of reaction sites thereby allowing a plurality of reactions to be performed. Thus, the devices can be used to assess circulating microRNAs in a multiplex fashion, including microRNAs isolated from vesicles. In an embodiment, the microfluidic device comprises (a) a first plurality of flow channels formed in an elastomeric substrate; (b) a second plurality of flow channels formed in the elastomeric substrate that intersect the first plurality of flow channels to define an array of reaction sites, each reaction site located at an intersection of one of the first and second flow channels; (c) a plurality of isolation valves disposed along the first and second plurality of flow channels and spaced between the reaction sites that can be actuated to isolate a solution within each of the reaction sites from solutions at other reaction sites, wherein the isolation valves comprise one or more control channels that each overlay and intersect one or more of the flow channels; and (d) means for simultaneously actuating the valves for isolating the reaction sites from each other. Various modifications to the basic structure of the device are envisioned within the scope of the invention. MicroRNAs can be detected in each of the reaction sites by using PCR methods. For example, the method can comprise the steps of the steps of: (i) providing a microfluidic device, the microfluidic device comprising: a first fluidic channel having a first end and a second end in fluid communication with each other through the channel; a plurality of flow channels, each flow channel terminating at a terminal wall; wherein each flow channel branches from and is in fluid communication with the first fluidic channel, wherein an aqueous fluid that enters one of the flow channels from the first fluidic channel can flow out of the flow channel only through the first fluidic channel; and, an inlet in fluid communication with the first fluidic channel, the inlet for introducing a sample fluid; wherein each flow channel is associated with a valve that when closed isolates one end of the flow channel from the first fluidic channel, whereby an isolated reaction site is formed between the valve and the terminal wall; a control channel; wherein each the valve is a deflectable membrane which is deflected into the flow channel associated with the valve when an actuating force is applied to the control channel, thereby closing the valve; and wherein when the actuating force is applied to the control channel a valve in each of the flow channels is closed, so as to produce the isolated reaction site in each flow channel; (ii) introducing the sample fluid into the inlet, the sample fluid filling the flow channels; (iii) actuating the valve to separate the sample fluid into the separate portions within the flow channels; (iv) amplifying the nucleic acid in the sample fluid; (v) analyzing the portions of the sample fluid to determine whether the amplifying produced the reaction. The sample fluid can contain an amplifiable nucleic acid target, e.g., a microRNA, and the conditions can be polymerase chain reaction (PCR) conditions, so that the reaction results in a PCR product being formed.

In an embodiment, the PCR used to detect microRNA is digital PCR, which is described by Brown, et al., U.S. Pat. No. 6,143,496, titled “Method of sampling, amplifying and quantifying segment of nucleic acid, polymerase chain reaction assembly having nanoliter-sized chambers and methods of filling chambers”, and by Vogelstein, et al, U.S. Pat. No. 6,446,706, titled “Digital PCR”, both of which are hereby incorporated by reference in their entirety. In digital PCR, a sample is partitioned so that individual nucleic acid molecules within the sample are localized and concentrated within many separate regions, such as the reaction sites of the microfluidic device described above. The partitioning of the sample allows one to count the molecules by estimating according to Poisson. As a result, each part will contain “0” or “1” molecules, or a negative or positive reaction, respectively. After PCR amplification, nucleic acids may be quantified by counting the regions that contain PCR end-product, positive reactions. In conventional PCR, starting copy number is proportional to the number of PCR amplification cycles. Digital PCR, however, is not dependent on the number of amplification cycles to determine the initial sample amount, eliminating the reliance on uncertain exponential data to quantify target nucleic acids and providing absolute quantification. Thus, the method can provide a sensitive approach to detecting microRNAs in a sample.

In one embodiment, a microfluidic device for isolating or detecting a vesicle comprises a channel of less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, of 60 mm in width, or between about 2-60, 3-50, 3-40, 3-30, 3-20, or 4-20 mm in width. The microchannel can have a depth of less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65 or 70 μm, or between about 10-70, 10-40, 15-35, or 20-30 μm. Furthermore, the microchannel can have a length of less than about 1, 2, 3, 3.5, 4, 4.5, 5, 5.5, 6, 6.5, 7, 7.5, 8, 8.5, 9, 9.5 or 10 cm. The microfluidic device can have grooves on its ceiling that are less than about 40, 41, 42, 43, 44, 45, 46, 47, 48, 49, 50, 51, 52, 53, 54, 55, 56, 57, 58, 59, 6, 65, 70, 75, or 80 μm wide, or between about 40-80, 40-70, 40-60 or 45-55 μm wide. The grooves can be less than about 1, 2, 3, 4, 5, 6, 7, 8, 9, 10, 11, 12, 13, 14, 15, 16, 17, 18, 19, 20, 25, 30, 35, 40, 45, or 50 μm deep, such as between about 1-50, 5-40, 5-30, 3-20 or 5-15 μm.

The microfluidic device can have one or more binding agents attached to a surface in a channel, or present in a channel. For example, the microchannel can have one or more capture agents, such as a capture agent for one or more general microvesicle antigen in Table 3 or a cell-of-origin or cancer related antigen in Table 4 or Table 5, including without limitation EpCam, CD9, PCSA, CD63, CD81, PSMA, B7H3, PSCA, ICAM, STEAP, KLK2, SSX2, SSX4, PBP, SPDEF, and/or EGFR. In one embodiment, a microchannel surface is treated with avidin and a capture agent, such as an antibody, that is biotinylated can be injected into the channel to bind the avidin. In other embodiments, the capture agents are present in chambers or other components of a microfluidic device. The capture agents can also be attached to beads that can be manipulated to move through the microfluidic channels. In one embodiment, the capture agents are attached to magnetic beads. The beads can be manipulated using magnets.

A biological sample can be flowed into the microfluidic device, or a microchannel, at rates such as at least about 1, 2, 3, 4, 5, 6, 7, 8, 9, 10, 11, 12, 13, 14, 15, 16, 17, 18, 19, 20, 25, 30, 35, 40, 45, or 50 μl per minute, such as between about 1-50, 5-40, 5-30, 3-20 or 5-15 μl per minute. One or more vesicles can be captured and directly detected in the microfluidic device. Alternatively, the captured vesicle may be released and exit the microfluidic device prior to analysis. In another embodiment, one or more captured vesicles are lysed in the microchannel and the lysate can be analyzed, e.g., to examine payload within the vesicles. Lysis buffer can be flowed through the channel and lyse the captured vesicles. For example, the lysis buffer can be flowed into the device or microchannel at rates such as at least about a, 2, 3, 4, 5, 6, 7, 8, 9, 10, 11, 12, 13, 14, 15, 16, 17, 18, 19, 20, 25, 26, 27, 28, 29, 30, 35, 40, 45, or 50 μl per minute, such as between about 1-50, 5-40, 10-30, 5-30 or 10-35 μl per minute. The lysate can be collected and analyzed, such as performing RT-PCR, PCR, mass spectrometry, Western blotting, or other assays, to detect one or more biomarkers of the vesicle.

The various isolation and detection systems described herein can be used to isolate or detect circulating biomarkers such as vesicles that are informative for diagnosis, prognosis, disease stratification, theranosis, prediction of responder/non-responder status, disease monitoring, treatment monitoring and the like as related to such diseases and disorders. Combinations of the isolation techniques are within the scope of the invention. In a non-limiting example, a sample can be run through a chromatography column to isolate vesicles based on a property such as size of electrophoretic motility, and the vesicles can then be passed through a microfluidic device. Binding agents can be used before, during or after these steps.

### Combined Isolation Methodology

One of skill will appreciate that various methods of sample treatment and isolating and concentrating circulating biomarkers such as vesicles can be combined as desired. For example, a biological sample can be treated to prevent aggregation, remove undesired particulate and/or deplete highly abundant proteins. The steps used can be chosen to optimize downstream analysis steps. Next, biomarkers such as vesicles can be isolated, e.g., by chromotography, centrifugation, density gradient, filtration, precipitation, or affinity techniques. Any number of the later steps can be combined, e.g., a sample could be subjected to one or more of chromotography, centrifugation, density gradient, filtration and precipitation in order to isolate or concentrate all or most microvesicles. In a subsequent step, affinity techniques, e.g., using binding agents to one or more target of interest, can be used to isolate or identify microvesicles carrying desired biomarker profiles. Microfluidic systems can be employed to perform some or all of these steps.

An exemplary isolation scheme for isolating and analysis of microvesicles includes the following: Plasma or serum collection→highly abundant protein removal→ultrafiltration→nanomembrane concentration→flow cytometry or particle-based assay.

Using the methods disclosed herein or known in the art, circulating biomarkers such as vesicles can be isolated or concentrated by at least about 2-fold, 3-fold, 1-fold, 2-fold, 3-fold, 4-fold, 5-fold, 6-fold, 7-fold, 8-fold, 9-fold, 10-fold, 12-fold, 15-fold, 20-fold, 25-fold, 30-fold, 35-fold, 40-fold, 45-fold, 50-fold, 55-fold, 60-fold, 65-fold, 70-fold, 75-fold, 80-fold, 90-fold, 95-fold, 100-fold, 110-fold, 120-fold, 125-fold, 130-fold, 140-fold, 150-fold, 160-fold, 170-fold, 175-fold, 180-fold, 190-fold, 200-fold, 225-fold, 250-fold, 275-fold, 300-fold, 325-fold, 350-fold, 375-fold, 400-fold, 425-fold, 450-fold, 475-fold, 500-fold, 525-fold, 550-fold, 575-fold, 600-fold, 625-fold, 650-fold, 675-fold, 700-fold, 725-fold, 750-fold, 775-fold, 800-fold, 825-fold, 850-fold, 875-fold, 900-fold, 925-fold, 950-fold, 975-fold, 1000-fold, 1500-fold, 2000-fold, 2500-fold, 3000-fold, 4000-fold, 5000-fold, 6000-fold, 7000-fold, 8000-fold, 9000-fold, or at least 10,000-fold. In some embodiments, the vesicles are isolated or concentrated concentrated by at least 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 orders of magnitude, or 10 orders of magnitude or more.

Once concentrated or isolated, the circulating biomarkers can be assessed, e.g., in order to characterize a phenotype as described herein. In some embodiments, the concentration or isolation steps themselves shed light on the phenotype of interest. For example, affinity methods can detect the presence or level of specific biomarkers of interest.

### Cell and Disease-Specific Vesicles

The bindings agent disclosed herein can be used to isolate or detect a vesicle, such as a cell-of-origin vesicle or vesicle with a specific biosignature. The binding agent can be used to isolate or detect a heterogeneous population of vesicles from a sample or can be used to isolate or detect a homogeneous population of vesicles, such as cell-of-origin specific vesicles with specific biosignatures, from a heterogeneous population of vesicles.

A homogeneous population of vesicles, such as cell-of-origin specific vesicles, can be analyzed and used to characterize a phenotype for a subject. Cell-of-origin specific vesicles are esicles derived from specific cell types, which can include, but are not limited to, cells of a specific tissue, cells from a specific tumor of interest or a diseased tissue of interest, circulating tumor cells, or cells of maternal or fetal origin. The vesicles may be derived from tumor cells or lung, pancreas, stomach, intestine, bladder, kidney, ovary, testis, skin, colorectal, breast, prostate, brain, esophagus, liver, placenta, or fetal cells. The isolated vesicle can also be from a particular sample type, such as urinary vesicle.

A cell-of-origin specific vesicle from a biological sample can be isolated using one or more binding agents that are specific to a cell-of-origin. Vesicles for analysis of a disease or condition can be isolated using one or more binding agent specific for biomarkers for that disease or condition.

A vesicle can be concentrated prior to isolation or detection of a cell-of-origin specific vesicle, such as through centrifugation, chromatography, or filtration, as described above, to produce a heterogeneous population of vesicles prior to isolation of cell-of-origin specific vesicles. Alternatively, the vesicle is not concentrated, or the biological sample is not enriched for a vesicle, prior to isolation of a cell-of-origin vesicle.

FIG. 1B illustrates a flowchart which depicts one method 100B for isolating or identifying a cell-of-origin specific vesicle. First, a biological sample is obtained from a subject in step 102. The sample can be obtained from a third party or from the same party performing the analysis. Next, cell-of-origin specific vesicles are isolated from the biological sample in step 104. The isolated cell-of-origin specific vesicles are then analyzed in step 106 and a biomarker or biosignature for a particular phenotype is identified in step 108. The method may be used for a number of phenotypes. In some embodiments, prior to step 104, vesicles are concentrated or isolated from a biological sample to produce a homogeneous population of vesicles. For example, a heterogeneous population of vesicles may be isolated using centrifugation, chromatography, filtration, or other methods as described above, prior to use of one or more binding agents specific for isolating or identifying vesicles derived from specific cell types.

A cell-of-origin specific vesicle can be isolated from a biological sample of a subject by employing one or more binding agents that bind with high specificity to the cell-of-origin specific vesicle. In some instances, a single binding agent can be employed to isolate a cell-of-origin specific vesicle. In other instances, a combination of binding agents may be employed to isolate a cell-of-origin specific vesicle. For example, at least 2, 3, 4, 5, 6, 7, 8, 9, 10, 11, 12, 13, 14, 15, 16, 17, 18, 19, 20, 25, 50, 75, or 100 different binding agents may be used to isolate a cell-of-origin vesicle. Therefore, a vesicle population (e.g., vesicles having the same binding agent profile) can be identified by using a single or a plurality of binding agents.

One or more binding agents can be selected based on their specificity for a target antigen(s) that is specific to a cell-of-origin, e.g., a cell-of-origin that is related to a tumor, autoimmune disease, cardiovascular disease, neurological disease, infection or other disease or disorder. The cell-of-origin can be from a cell that is informative for a diagnosis, prognosis, disease stratification, theranosis, prediction of responder/non-responder status, disease monitoring, treatment monitoring and the like as related to such diseases and disorders. The cell-of-origin can also be from a cell useful to discover biomarkers for use thereto. Non-limiting examples of antigens which may be used singularly, or in combination, to isolate a cell-of-origin specific vesicle, disease specific vesicle, or tumor specific vesicle, are shown in FIG. 1 of International Patent Application Serial No. PCT/US2011/031479, entitled “Circulating Biomarkers for Disease” and filed Apr. 6, 2011, which application is incorporated by reference in its entirety herein, and are also described herein. The antigen can comprise membrane bound antigens which are accessible to binding agents. The antigen can be a biomarker related to characterizing a phenotype.

One of skill will appreciate that any applicable antigen that can be used to isolate an informative vesicle is contemplated by the invention. Binding agents, e.g., antibodies, aptamers and lectins, can be chosen that recognize surface antigens and/or fragments thereof, as outlined herein. The binding agents can recognize antigens specific to the desired cell type or location and/or recognize biomarkers associated with the desired cells. The cells can be, e.g., tumor cells, other diseased cells, cells that serve as markers of disease such as activated immune cells, etc. One of skill will appreciate that binding agents for any cells of interest can be useful for isolating vesicles associated with those cells. One of skill will further appreciate that the binding agents disclosed herein can be used for detecting vesicles of interest. As a non-limiting example, a binding agent to a vesicle biomarker can be labeled directly or indirectly in order to detect vesicles bound by one of more of the same or different binding agents.

A number of targets for binding agents useful for binding to vesicles associated with cancer, autoimmune diseases, cardiovascular diseases, neurological diseases, infection or other disease or disorders are presented in Table 4. A vesicle derived from a cell associated with one of the listed disorders can be characterized using one of the antigens in the table. The binding agent, e.g., an antibody or aptamer, can recognize an epitope of the listed antigens, a fragment thereof, or binding agents can be used against any appropriate combination. Other antigens associated with the disease or disorder can be recognized as well in order to characterize the vesicle. One of skill will appreciate that any applicable antigen that can be used to assess an informative vesicle is contemplated by the invention for isolation, capture or detection in order to characterize a vesicle.

TABLE 4
Illustrative Antigens for Use in Characterizing Various Diseases and Disorders

| Disease or disorder | Target |
|---|---|
| Breast cancer, e.g., glandular or stromal cells | BCA-225, hsp70, MART1, ER, VEGFA, Class III b- |
| | tubulin, HER2/neu (for Her2+ breast cancer), GPR30, |
| | ErbB4 (JM) isoform, MPR8, MISIIR |
| Breast cancer | CD9, MIS Rii, ER, CD63, MUC1, HER3, STAT3, |
| | VEGFA, BCA, CA125, CD24, EPCAM, ERB B4 |
| Breast cancer | BCA-225, hsp70, MART1, ER, VEGFA, Class III b- |
| | tubulin, HER2/neu (e.g., for Her2+ breast cancer), |
| | GPR30, ErbB4 (JM) isoform, MPR8, MISIIR, CD9, |
| | EphA2, EGFR, B7H3, PSM, PCSA, CD63, STEAP, |
| | CD81, ICAM1, A33, DR3, CD66e, MFG-E8, TROP-2, |
| | Mammaglobin, Hepsin, NPGP/NPFF2, PSCA, 5T4, |
| | NGAL, EpCam, neurokinin receptor-1 (NK-1 or NK- |
| | 1R), NK-2, Pai-1, CD45, CD10, HER2/ERBB2, |
| | AGTR1, NPY1R, MUC1, ESA, CD133, GPR30, |
| | BCA225, CD24, CA15.3 (MUC1 secreted), CA27.29 |
| | (MUC1 secreted), NMDAR1, NMDAR2, MAGEA, |
| | CTAG1B, NY-ESO-1, SPB, SPC, NSE, PGP9.5, a |
| | progesterone receptor (PR) or its isoform (PR(A) or |
| | PR(B)), P2RX7, NDUFB7, NSE, GAL3, osteopontin, |
| | CHI3L1, IC3b, mesothelin, SPA, AQP5, GPCR, |
| | hCEA-CAM, PTP IA-2, CABYR, TMEM211, |
| | ADAM28, UNC93A, MUC17, MUC2, IL10R-beta, |
| | BCMA, HVEM/TNFRSF14, Trappin-2 Elafin, |
| | ST2/IL1 R4, TNFRF14, CEACAM1, TPA1, LAMP, |
| | WF, WH1000, PECAM, BSA, TNF |
| Breast cancer | CD10, NPGP/NPFF2, HER2/ERBB2, AGTR1, |
| | NPY1R, neurokinin receptor-1 (NK-1 or NK-1R), NK- |
| | 2, MUC1, ESA, CD133, GPR30, BCA225, CD24, |
| | CA15.3 (MUC1 secreted), CA27.29 (MUC1 secreted), |
| | NMDAR1, NMDAR2, MAGEA, CTAG1B, NY-ESO-1 |
| Breast cancer | SPB, SPC, NSE, PGP9.5, CD9, P2RX7, NDUFB7, |
| | NSE, GAL3, osteopontin, CHI3L1, EGFR, B7H3, |
| | IC3b, MUC1, mesothelin, SPA, PCSA, CD63, STEAP, |
| | AQP5, CD81, DR3, PSM, GPCR, EphA2, hCEA- |
| | CAM, PTP IA-2, CABYR, TMEM211, ADAM28, |
| | UNC93A, A33, CD24, CD10, NGAL, EpCam, |
| | MUC17, TROP-2, MUC2, IL10R-beta, BCMA, |
| | HVEM/TNFRSF14, Trappin-2 Elafin, ST2/IL1 R4, |
| | TNFRF14, CEACAM1, TPA1, LAMP, WF, WH1000, |
| | PECAM, BSA, TNFR |
| Breast cancer | BRCA, MUC-1, MUC 16, CD24, ErbB4, ErbB2 |
| | (HER2), ErbB3, HSP70, Mammaglobin, PR, PR(B), |
| | VEGFA |
| Ovarian Cancer | CA125, VEGFR2, HER2, MISIIR, VEGFA, CD24, c- |
| | reactive protein EGFR, EGFRvIII, apolipoprotein AI, |
| | apolipoprotein CIII, myoglobin, tenascin C, MSH6, |
| | claudin-3, claudin-4, caveolin-1, coagulation factor III, |
| | CD9, CD36, CD37, CD53, CD63, CD81, CD136, |
| | CD147, Hsp70, Hsp90, Rab13, Desmocollin-1, EMP- |
| | 2, CK7, CK20, GCDF15, CD82, Rab-5b, Annexin V, |
| | MFG-E8, HLA-DR, CD95 |
| Lung Cancer | CYFRA21-1, TPA-M, TPS, CEA, SCC-Ag, XAGE- |
| | 1b, HLA Class 1, TA-MUC1, KRAS, hENT1, kinin |
| | B1 receptor, kinin B2 receptor, TSC403, HTI56, DC- |
| | LAMP |
| Lung Cancer | SPB, SPC, PSP9.5, NDUFB7, gal3-b2c10, iC3b, |
| | MUC1, GPCR, CABYR and muc17 |
| Colorectal Cancer | CEA, MUC2, GPA33, CEACAM5, ENFB1, CCSA-3, |
| | CCSA-4, ADAM10, CD44, NG2, ephrin B1, |
| | plakoglobin, galectin 4, RACK1, tetraspanin-8, FASL, |
| | A33, CEA, EGFR, dipeptidase 1, PTEN, Na(+)- |
| | dependent glucose transporter, UDP- |
| | glucuronosyltransferase 1A, TMEM211, CD24 |
| Prostate Cancer | PSA, TMPRSS2, FASLG, TNFSF10, PSMA, NGEP, |
| | Il-7RI, CSCR4, CysLT1R, TRPM8, Kv1.3, TRPV6, |
| | TRPM8, PSGR, MISIIR, galectin-3, PCA3, |
| | TMPRSS2:ERG |
| Brain Cancer | PRMT8, BDNF, EGFR, DPPX, Elk, Densin-180, |
| | BAI2, BAI3 |
| Blood Cancer (hematological malignancy) | CD44, CD58, CD31, CD11a, CD49d, GARP, BTS, |
| | Raftlin |
| Melanoma | DUSP1, TYRP1, SILV, MLANA, MCAM, CD63, |
| | Alix, hsp70, meosin, p120 catenin, PGRL, syntaxin |
| | binding protein 1 & 2, caveolin |
| Liver Cancer (hepatocellular carcinoma) | HBxAg, HBsAg, NLT |
| Cervical Cancer | MCT-1, MCT-2, MCT-4 |
| Endometrial Cancer | Alpha V Beta 6 integrin |
| Psoriasis | flt-1, VPF receptors, kdr |
| Autoimmune Disease | Tim-2 |
| Irritable Bowel Disease (IBD or Syndrome (IBS) | IL-16, IL-1beta, IL-12, TNF-alpha, interferon-gamma, |
| | IL-6, Rantes, II-12, MCP-1, 5HT |
| Diabetes, e.g., pancreatic cells | IL-6, CRP, RBP4 |
| Barrett's Esophagus | p53, MUC1, MUC6 |
| Fibromyalgia | neopterin, gp130 |
| Benign Prostatic Hyperplasia (BPH) | KIA1, intact fibronectin |
| Multiple Sclerosis | B7, B7-2, CD-95 (fas), Apo-1/Fas |
| Parkinson's Disease | PARK2, ceruloplasmin, VDBP, tau, DJ-1 |
| Rheumatic Disease | Citrulinated fibrin a-chain, CD5 antigen-like |
| | fibrinogen fragment D, CD5 antigen-like fibrinogen |
| | fragment B, TNF alpha |
| Alzheimer's Disease | APP695, APP751 or APP770, BACE1, cystatin C, |
| | amyloid β, T-tau, complement factor H, alpha-2- |
| | macroglobulin |
| Head and Neck Cancer | EGFR, EphB4, Ephrin B2 |
| Gastrointestinal Stromal Tumor (GIST) | c-kit PDGFRA, NHE-3 |
| Renal Cell Carcinoma | c PDGFRA, VEGF, HIF 1 alpha |
| Schizophrenia | ATP5B, ATP5H, ATP6V1B, DNM1 |
| Peripheral Neuropathic Pain | OX42, ED9 |
| Chronic Neuropathic Pain | chemokine receptor (CCR2/4) |
| Prion Disease | PrPSc, 14-3-3 zeta, S-100, AQP4 |
| Stroke | S-100, neuron specific enolase, PARK7, NDKA, |
| | ApoC-I, ApoC-III, SAA or AT-III fragment, Lp- |
| | PLA2, hs-CRP |
| Cardiovascular Disease | FATP6 |
| Esophageal Cancer | CaSR |
| Tuberculosis | antigen 60, HSP, Lipoarabinomannan, Sulfolipid, |
| | antigen of acylated trehalose family, DAT, TAT, |
| | Trehalose 6,6-dimycolate (cord-factor) antigen |
| HIV | gp41, gp120 |
| Autism | VIP, PACAP, CGRP, NT3 |
| Asthma | YKL-40, S-nitrosothiols, SSCA2, PAI, amphiregulin, |
| | periostin |
| Lupus | TNFR |
| Cirrhosis | NLT, HBsAg |
| Influenza | hemagglutinin, neurominidase |
| Vulnerable Plaque | Alpha v. Beta 3 integrin, MMP9 |

The foregoing Table 4, as well as other biomarker lists disclosed here are illustrative, and Applicants contemplate incorporating various biomarkers disclosed across different disease states or conditions. For example, method of the invention may use various biomarkers across different diseases or conditions, where the biomarkers are useful for providing a diagnostic, prognostic or theranostic signature. In one embodiment, angiogenic, inflammatory or immune-associated antigens (or biomarkers) disclosed herein or know in the art can be used in methods of the invention to screen a biological sample in identification of a biosignature. Indeed, the flexibility of Applicants' multiplex approach to assessing microvesicle populations facilitates assessing various markers (and in some instances overlapping markers) for different conditions or diseases whose etiology necessarily may share certain cellular and biological mechanisms, e.g., different cancers implicating biomarkers for angiogenesis, or immune response regulation or modulation. The combination of such overlapping biomarkers with tissue or cell-specific biomarkers, along with microvesicle-associated biomarkers provides a powerful series of tools for practicing the methods and compositions of the invention.

A cell-of-origin specific vesicle may be isolated using novel binding agents, using methods as described herein. Furthermore, a cell-of-origin specific vesicle can also be isolated from a biological sample using isolation methods based on cellular binding partners or binding agents of such vesicles. Such cellular binding partners can include but are not limited to peptides, proteins, RNA, DNA, apatmers, cells or serum-associated proteins that only bind to such vesicles when one or more specific biomarkers are present. Isolation or deteciton of a cell-of-origin specific vesicle can be carried out with a single binding partner or binding agent, or a combination of binding partners or binding agents whose singular application or combined application results in cell-of-origin specific isolation or detection. Non-limiting examples of such binding agents are provided in FIG. 2 of International Patent Application Serial No. PCT/US2011/031479, entitled “Circulating Biomarkers for Disease” and filed Apr. 6, 2011, which application is incorporated by reference in its entirety herein. For example, a vesicle for characterizing breast cancer can be isolated with one or more binding agents including, but not limited to, estrogen, progesterone, trastuzumab, CCND1, MYC PNA, IGF-1 PNA, MYC PNA, SC4 aptamer (Ku), AII-7 aptamer (ERB2), Galectin-3, mucin-type O-glycans, L-PHA, Galectin-9, or any combination thereof.

A binding agent may also be used for isolating or detecting a cell-of-origin specific vesicle based on: i) the presence of antigens specific for cell-of-origin specific vesicles; ii) the absence of markers specific for cell-of-origin specific vesicles; or iii) expression levels of biomarkers specific for cell-of-origin specific vesicles. A heterogeneous population of vesicles can be applied to a surface coated with specific binding agents designed to rule out or identify the cell-of-origin characteristics of the vesicles. Various binding agents, such as antibodies, can be arrayed on a solid surface or substrate and the heterogeneous population of vesicles is allowed to contact the solid surface or substrate for a sufficient time to allow interactions to take place. Specific binding or nonbinding to given antibody locations on the array surface or substrate can then serve to identify antigen specific characteristics of the vesicle population that are specific to a given cell-of-origin. That is, binding events can signal the presence of a vesicle having an antigen recognized by the bound antibody. Conversely, lack of binding events can signal the absence of vesicles having an antigen recognized by the bound antibody.

A cell-of-origin specific vesicle can be enriched or isolated using one or more binding agents using a magnetic capture method, fluorescence activated cell sorting (FACS) or laser cytometry as described above. Magnetic capture methods can include, but are not limited to, the use of magnetically activated cell sorter (MACS) microbeads or magnetic columns. Examples of immunoaffinity and magnetic particle methods that can be used are described in U.S. Pat. Nos. 4,551,435, 4,795,698, 4,925,788, 5,108,933, 5,186,827, 5,200,084 or 5,158,871. A cell-of-origin specific vesicle can also be isolated following the general methods described in U.S. Pat. No. 7,399,632, by using combination of antigens specific to a vesicle.

Any other appropriate method for isolating or otherwise enriching the cell-of-origin specific vesicles with respect to a biological sample may also be used in combination with the present invention. For example, size exclusion chromatography such as gel permeation columns, centrifugation or density gradient centrifugation, and filtration methods can be used in combination with the antigen selection methods described herein. The cell-of-origin specific vesicles may also be isolated following the methods described in Koga et al., Anticancer Research, 25:3703-3708 (2005), Taylor et al., Gynecologic Oncology, 110:13-21 (2008), Nanjee et al., Clin Chem, 2000; 46:207-223 or U.S. Pat. No. 7,232,653.

Vesicles can be isolated and/or detected to provide diagnosis, prognosis, disease stratification, theranosis, prediction of responder/non-responder status, disease monitoring, treatment monitoring and the like. In one embodiment, vesicles are isolated from cells having a disease or disorder, e.g., cells derived from a tumor or malignant growth, a site of autoimmune disease, cardiovascular disease, neurological disease, or infection. In some embodiments, the isolated vesicles are derived from cells related to such diseases and disorders, e.g., immune cells that play a role in the etiology of the disease and whose analysis is informative for a diagnosis, prognosis, disease stratification, theranosis, prediction of responder/non-responder status, disease monitoring, treatment monitoring and the like as relates to such diseases and disorders. The vesicles are further useful to discover novel biomarkers. By identifying biomarkers associated with vesicles, isolated vesicles can be assessed for characterizing a phenotype as described herein.

In some embodiments, methods of the invention are directed to characterizing presence of a cancer or likelihood of a cancer occurring in an individual by assessing one or more microvesicle population present in a biological sample from an individual. Microvesicles can be isolated using one or more processes disclosed herein or practiced in the art.

Such microvesicles populations can each separately or collectively provide a disease phenotype characterization for the individual by comparing the biomarker profile, or biosignature, for the microvesicle population(s) with a reference sample to provide a diagnostic, prognostic or theranostic characterization for the test sample.

The vesicle population(s) can be assessed from various biological samples and bodily fluids such as disclosed herein.

### Biomarker Assessment

In an aspect of the invention, a phenotype of a subject is characterized by analyzing a biological sample and determining the presence, level, amount, or concentration of one or more populations of circulating biomarkers in the sample, e.g., circulating vesicles, proteins or nucleic acids. In embodiments, characterization includes determining whether the circulating biomarkers in the sample are altered as compared to a reference, which can also be referred to a standard or a control. An alteration can include any measurable difference between the sample and the reference, including without limitation an absolute presence or absence, a quantitative level, a relative level compared to a reference, e.g., the level of all vesicles present, the level of a housekeeping marker, and/or the level of a spiked-in marker, an elevated level, a decreased level, overexpression, underexpression, differential expression, a mutation or other altered sequence, a modification (glycosylation, phosphorylation, epigenetic change) and the like. In some embodiments, circulating biomarkers are purified or concentrated from a sample prior to determining their amount. Unless otherwise specified, “purified” or “isolated” as used herein refer to partial or complete purification or isolation. In other embodiments, circulating biomarkers are directly assessed from a sample, without prior purification or concentration. Circulating vesicles can be cell-of-origin specific vesicles or vesicles with a specific biosignature. A biosignature includes specific pattern of biomarkers, e.g., patterns of biomarkers indicative of a phenotype that is desireable to detect, such as a disease phenotype. The biosignature can comprise one or more circulating biomarkers. A biosignature can be used when characterizing a phenotype, such as a diagnosis, prognosis, theranosis, or prediction of responder/non-responder status. In some embodiments, the biosignature is used to determine a physiological or biological state, such as pregnancy or the stage of pregnancy. The biosignature can also be used to determine treatment efficacy, stage of a disease or condition, or progression of a disease or condition. For example, the amount of one or more vesicles can be proportional or inversely proportional to an increase in disease stage or progression. The detected amount of vesicles can also be used to monitor progression of a disease or condition or to monitor a subject's response to a treatment.

The circulating biomarkers can be evaluated by comparing the level of circulating biomarkers with a reference level or value. The reference value can be particular to physical or temporal endpoint. For example, the reference value can be from the same subject from whom a sample is assessed, or the reference value can be from a representative population of samples (e.g., samples from normal subjects not exhibiting a symptom of disease). Therefore, a reference value can provide a threshold measurement which is compared to a subject sample's readout for a biosignature assayed in a given sample. Such reference values may be set according to data pooled from groups of sample corresponding to a particular cohort, including but not limited to age (e.g., newborns, infants, adolescents, young, middle-aged adults, seniors and adults of varied ages), racial/ethnic groups, normal versus diseased subjects, smoker v. non-smoker, subject receiving therapy versus untreated subject, different time points of treatment for a particular individual or group of subjects similarly diagnosed or treated or combinations thereof. Furthermore, by determining a biosignature at different timepoints of treatment for a particular individual, the individual's response to the treatment or progression of a disease or condition for which the individual is being treated for, can be monitored.

A reference value may be based on samples assessed from the same subject so to provide individualized tracking. In some embodiments, frequent testing of a biosignature in samples from a subject provides better comparisons to the reference values previously established for that subject. Such time course measurements are used to allow a physician to more accurately assess the subject's disease stage or progression and therefore inform a better decision for treatment. In some cases, the variance of a biosignature is reduced when comparing a subject's own biosignature over time, thus allowing an individualized threshold to be defined for the subject, e.g., a threshold at which a diagnosis is made. Temporal intrasubject variation allows each individual to serve as their own longitudinal control for optimum analysis of disease or physiological state. As an illustrative example, consider that the level of vesicles derived from prostate cells is measured in a subject's blood over time. A spike in the level of prostate-derived vesicles in the subject's blood can indicate hyperproliferation of prostate cells, e.g., due to prostate cancer.

Reference values can be established for unaffected individuals (of varying ages, ethnic backgrounds and sexes) without a particular phenotype by determining the biosignature of interest in an unaffected individual. For example, a reference value for a reference population can be used as a baseline for detection of one or more circulating biomarker populations in a test subject. If a sample from a subject has a level or value that is similar to the reference, the subject can be identified to not have the disease, or of having a low likelihood of developing a disease.

Alternatively, reference values or levels can be established for individuals with a particular phenotype by determining the amount of one or more populations of vesicles in an individual with the phenotype. In addition, an index of values can be generated for a particular phenotype. For example, different disease stages can have different values, such as obtained from individuals with the different disease stages. A subject's value can be compared to the index and a diagnosis or prognosis of the disease can be determined, such as the disease stage or progression wherein the subject's levels most closely correlate with the index. In other embodiments, an index of values is generated for therapeutic efficacies. For example, the level of vesicles of individuals with a particular disease can be generated and noted what treatments were effective for the individual. The levels can be used to generate values of which is a subject's value is compared, and a treatment or therapy can be selected for the individual, e.g., by predicting from the levels whether the subject is likely to be a responder or non-responder for a treatment.

In some embodiments, a reference value is determined for individuals unaffected with a particular cancer, by isolating or detecting circulating biomarkers with an antigen that specifically targets biomarkers for the particular cancer. As a non-limiting example, individuals with varying stages of colorectal cancer and noncancerous polyps can be surveyed using the same techniques described for unaffected individuals and the levels of circulating vesicles for each group can be determined. In some embodiments, the levels are defined as means±standard deviations from at least two separate experiments, performed in at least duplicate or triplicate. Comparisons between these groups can be made using statistical tests to determine statistical significance of distinguishing biomarkers observed. In some embodiments, statistical significance is determined using a parametric statistical test. The parametric statistical test can comprise, without limitation, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than 0.05, 0.01, 0.005, 0.001, 0.0005, or 0.0001. The p-values can also be corrected for multiple comparisons, e.g., using a Bonferroni correction, a modification thereof, or other technique known to those in the art, e.g., the Hochberg correction, Holm-Bonferroni correction, \check{S}idák correction, Dunnett's correction or Tukey's multiple comparisons. In some embodiments, an ANOVA is followed by Tukey's correction for post-test comparing of the biomarkers from each population. A biosignature comprising more than one marker can be evaluated using multivariate modeling techniques to build a classifier using techniques described herein or known in the art.

Reference values can also be established for disease recurrence monitoring (or exacerbation phase in MS), for therapeutic response monitoring, or for predicting responder/non-responder status.

In some embodiments, a reference value for vesicles is determined using an artificial vesicle, also referred to herein as a synthetic vesicle. Methods for manufacturing artificial vesicles are known to those of skill in the art, e.g., using liposomes. Artificial vesicles can be manufactured using methods disclosed in US20060222654 and U.S. Pat. No. 4,448,765, which are incorporated herein by reference in its entirety. Artificial vesicles can be constructed with known markers to facilitate capture and/or detection. In some embodiments, artificial vesicles are spiked into a bodily sample prior to processing. The level of intact synthetic vesicle can be tracked during processing, e.g., using filtration or other isolation methods disclosed herein, to provide a control for the amount of vesicles in the initial versus processed sample. Similarly, artificial vesicles can be spiked into a sample before or after any processing steps. In some embodiments, artificial vesicles are used to calibrate equipment used for isolation and detection of vesicles.

Artificial vesicles can be produced and used a control to test the viability of an assay, such as a bead-based assay. The artificial vesicle can bind to both the beads and to the detection antibodies. Thus, the artificial vesicle contains the amino acid sequence/conformation that each of the antibodies binds. The artificial vesicle can comprise a purified protein or a synthetic peptide sequence to which the antibody binds. The artificial vesicle could be a bead, e.g., a polystyrene bead, that is capable of having biological molecules attached thereto. If the bead has an available carboxyl group, then the protein or peptide could be attached to the bead via an available amine group, such as using carbodiimide coupling.

In another embodiment, the artificial vesicle can be a polystyrene bead coated with avidin and a biotin is placed on the protein or peptide of choice either at the time of synthesis or via a biotin-maleimide chemistry. The proteins/peptides to be on the bead can be mixed together in ratio specific to the application the artificial vesicle is being used for, and then conjugated to the bead. These artificial vesicles can then serve as a link between the capture beads and the detection antibodies, thereby providing a control to show that the components of the assay are working properly.

The value can be a quantitative or qualitative value. The value can be a direct measurement of the level of vesicles (example, mass per volume), or an indirect measure, such as the amount of a specific biomarker. The value can be a quantitative, such as a numerical value. In other embodiments, the value is qualitiative, such as no vesicles, low level of vesicles, medium level, high level of vesicles, or variations thereof.

The reference value can be stored in a database and used as a reference for the diagnosis, prognosis, theranosis, disease stratification, disease monitoring, treatment monitoring or prediction of non-responder/responder status of a disease or condition based on the level or amount of circulating biomarkers, such as total amount of vesicles or microRNA, or the amount of a specific population of vesicles or microRNA, such as cell-of-origin specific vesicles or microRNA or microRNA from vesicles with a specific biosignature. In an illustrative example, consider a method of determining a diagnosis for a cancer. Vesicles or other circulating biomarkers from reference subjects with and without the cancer are assessed and stored in the database. The reference subjects provide biosignature indicative of the cancer or of another state, e.g., a healthy state. A sample from a test subject is then assayed and the microRNA biosignature is compared against those in the database. If the subject's biosignature correlates more closely with reference values indicative of cancer, a diagnosis of cancer may be made. Conversely, if the subject's biosignature correlates more closely with reference values indicative of a healthy state, the subject may be determined to not have the disease. One of skill will appreciate that this example is non-limiting and can be expanded for assessing other phenotypes, e.g., other diseases, prognosis, theranosis, disease stratification, disease monitoring, treatment monitoring or prediction of non-responder/responder status, and the like.

A biosignature for characterizing a phenotype can be determined by detecting circulating biomarkers such as vesicles, including biomarkers associate with vesicles such as surface antigens or payload. The payload, e.g., protein or species of RNA such as mRNA or microRNA, can be assessed within a vesicle. Alternately, the payload in a sample is analyzed to characterize the phenotype without isolating the payload from the vesicles. Many analytical techniques are available to assess vesicles. In some embodiments, vesicle levels are characterized using mass spectrometry, flow cytometry, immunocytochemical staining, Western blotting, electrophoresis, chromatography or x-ray crystallography in accordance with procedures known in the art. For example, vesicles can be characterized and quantitatively measured using flow cytometry as described in Clayton et al., Journal of Immunological Methods 2001; 163-174, which is herein incorporated by reference in its entirety. Vesicle levels may be determined using binding agents as described above. For example, a binding agent to vesicles can be labeled and the label detected and used to determine the amount of vesicles in a sample. The binding agent can be bound to a substrate, such as arrays or particles, such as described above. Alternatively, the vesicles may be labeled directly.

Electrophoretic tags or eTags can be used to determine the amount of vesicles. eTags are small fluorescent molecules linked to nucleic acids or antibodies and are designed to bind one specific nucleic acid sequence or protein, respectively. After the eTag binds its target, an enzyme is used to cleave the bound eTag from the target. The signal generated from the released eTag, called a “reporter,” is proportional to the amount of target nucleic acid or protein in the sample. The eTag reporters can be identified by capillary electrophoresis. The unique charge-to-mass ratio of each eTag reporter—that is, its electrical charge divided by its molecular weight—makes it show up as a specific peak on the capillary electrophoresis readout. Thus by targeting a specific biomarker of a vesicle with an eTag, the amount or level of vesicles can be determined.

The vesicle level can determined from a heterogeneous population of vesicles, such as the total population of vesicles in a sample. Alternatively, the vesicles level is determined from a homogenous population, or substantially homogenous population of vesicles, such as the level of specific cell-of-origin vesicles, such as vesicles from prostate cancer cells. In yet other embodiments, the level is determined for vesicles with a particular biomarker or combination of biomarkers, such as a biomarker specific for prostate cancer. Determining the level vesicles can be performed in conjunction with determining the biomarker or combination of biomarkers of a vesicle. Alternatively, determining the amount of vesicle may be performed prior to or subsequent to determining the biomarker or combination of biomarkers of the vesicles.

Determining the amount of vesicles can be assayed in a multiplexed manner. For example, determining the amount of more than one population of vesicles, such as different cell-of-origin specific vesicles with different biomarkers or combination of biomarkers, can be performed, such as those disclosed herein.

Performance of a diagnostic or related test is typically assessed using statistical measures. The performance of the characterization can be assessed by measuring sensitivity, specificity and related measures. For example, a level of circulating biomarkers of interest can be assayed to characterize a phenotype, such as detecting a disease. The sensitivity and specificity of the assay to detect the disease is determined.

A true positive is a subject with a characteristic, e.g., a disease or disorder, correctly identified as having the characteristic. A false positive is a subject without the characteristic that the test improperly identifies as having the characteristic. A true negative is a subject without the characteristic that the test correctly identifies as not having the characteristic. A false negative is a person with the characteristic that the test improperly identifies as not having the characteristic. The ability of the test to distinguish between these classes provides a measure of test performance.

The specificity of a test is defined as the number of true negatives divided by the number of actual negatives (i.e., sum of true negatives and false positives). Specificity is a measure of how many subjects are correctly identified as negatives. A specificity of 100% means that the test recognizes all actual negatives—for example, all healthy people will be recognized as healthy. A lower specificity indicates that more negatives will be determined as positive.

The sensitivity of a test is defined as the number of true positives divided by the number of actual positives (i.e., sum of true positives and false negatives). Sensitivity is a measure of how many subjects are correctly identified as positives. A sensitivity of 100% means that the test recognizes all actual positives—for example, all sick people will be recognized as sick. A lower sensitivity indicates that more positives will be missed by being determined as negative.

The accuracy of a test is defined as the number of true positives and true negatives divided by the sum of all true and false positives and all true and false negatives. It provides one number that combines sensitivity and specificity measurements.

Sensitivity, specificity and accuracy are determined at a particular discrimination threshold value. For example, a common threshold for prostate cancer (PCa) detection is 4 ng/mL of prostate specific antigen (PSA) in serum. A level of PSA equal to or above the threshold is considered positive for PCa and any level below is considered negative. As the threshold is varied, the sensitivity and specificity will also vary. For example, as the threshold for detecting cancer is increased, the specificity will increase because it is harder to call a subject positive, resulting in fewer false positives. At the same time, the sensitivity will decrease. A receiver operating characteristic curve (ROC curve) is a graphical plot of the true positive rate (i.e., sensitivity) versus the false positive rate (i.e., 1−specificity) for a binary classifier system as its discrimination threshold is varied. The ROC curve shows how sensitivity and specificity change as the threshold is varied. The Area Under the Curve (AUC) of an ROC curve provides a summary value indicative of a test's performance over the entire range of thresholds. The AUC is equal to the probability that a classifier will rank a randomly chosen positive sample higher than a randomly chosen negative sample. An AUC of 0.5 indicates that the test has a 50% chance of proper ranking, which is equivalent to no discriminatory power (a coin flip also has a 50% chance of proper ranking) An AUC of 1.0 means that the test properly ranks (classifies) all subjects. The AUC is equivalent to the Wilcoxon test of ranks.

A biosignature according to the invention can be used to characterize a phenotype with at least 50, 51, 52, 53, 54, 55, 56, 57, 58, 59, 60, 61, 62, 63, 64, 65, 66, 67, 68, 69, or 70% sensitivity, such as with at least 71, 72, 73, 74, 75, 76, 77, 78, 79, 80, 81, 82, 83, 84, 85, 86, or 87% sensitivity. In some embodiments, the phenotype is characterized with at least 87.1, 87.2, 87.3, 87.4, 87.5, 87.6, 87.7, 87.8, 87.9, 88.0, or 89% sensitivity, such as at least 90% sensitivity. The phenotype can be characterized with at least 91, 92, 93, 94, 95, 96, 97, 98, 99 or 100% sensitivity.

A biosignature according to the invention can be used to characterize a phenotype of a subject with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

A biosignature according to the invention can be used to characterize a phenotype of a subject, e.g., based on a level of a circulating biomarker or other characteristic, with at least 50% sensitivity and at least 60, 65, 70, 75, 80, 85, 90, 95, 99, or 100% specificity; at least 55% sensitivity and at least 60, 65, 70, 75, 80, 85, 90, 95, 99, or 100% specificity; at least 60% sensitivity and at least 60, 65, 70, 75, 80, 85, 90, 95, 99, or 100% specificity; at least 65% sensitivity and at least 60, 65, 70, 75, 80, 85, 90, 95, 99, or 100% specificity; at least 70% sensitivity and at least 60, 65, 70, 75, 80, 85, 90, 95, 99, or 100% specificity; at least 75% sensitivity and at least 60, 65, 70, 75, 80, 85, 90, 95, 99, or 100% specificity; at least 80% sensitivity and at least 60, 65, 70, 75, 80, 85, 90, 95, 99, or 100% specificity; at least 85% sensitivity and at least 60, 65, 70, 75, 80, 85, 90, 95, 99, or 100% specificity; at least 86% sensitivity and at least 60, 65, 70, 75, 80, 85, 90, 95, 99, or 100% specificity; at least 87% sensitivity and at least 60, 65, 70, 75, 80, 85, 90, 95, 99, or 100% specificity; at least 88% sensitivity and at least 60, 65, 70, 75, 80, 85, 90, 95, 99, or 100% specificity; at least 89% sensitivity and at least 60, 65, 70, 75, 80, 85, 90, 95, 99, or 100% specificity; at least 90% sensitivity and at least 60, 65, 70, 75, 80, 85, 90, 95, 99, or 100% specificity; at least 91% sensitivity and at least 60, 65, 70, 75, 80, 85, 90, 95, 99, or 100% specificity; at least 92% sensitivity and at least 60, 65, 70, 75, 80, 85, 90, 95, 99, or 100% specificity; at least 93% sensitivity and at least 60, 65, 70, 75, 80, 85, 90, 95, 99, or 100% specificity; at least 94% sensitivity and at least 60, 65, 70, 75, 80, 85, 90, 95, 99, or 100% specificity; at least 95% sensitivity and at least 60, 65, 70, 75, 80, 85, 90, 95, 99, or 100% specificity; at least 96% sensitivity and at least 60, 65, 70, 75, 80, 85, 90, 95, 99, or 100% specificity; at least 97% sensitivity and at least 60, 65, 70, 75, 80, 85, 90, 95, 99, or 100% specificity; at least 98% sensitivity and at least 60, 65, 70, 75, 80, 85, 90, 95, 99, or 100% specificity; at least 99% sensitivity and at least 60, 65, 70, 75, 80, 85, 90, 95, 99, or 100% specificity; or substantially 100% sensitivity and at least 60, 65, 70, 75, 80, 85, 90, 95, 99, or 100% specificity.

A biosignature according to the invention can be used to characterize a phenotype of a subject with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97% accuracy, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% accuracy.

In some embodiments, a biosignature according to the invention is used to characterize a phenotype of a subject with an AUC of at least 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, or 0.97, such as with at least 0.971, 0.972, 0.973, 0.974, 0.975, 0.976, 0.977, 0.978, 0.978, 0.979, 0.980, 0.981, 0.982, 0.983, 0.984, 0.985, 0.986, 0.987, 0.988, 0.989, 0.99, 0.991, 0.992, 0.993, 0.994, 0.995, 0.996, 0.997, 0.998, 0.999 or 1.00.

Furthermore, the confidence level for determining the specificity, sensitivity, accuracy or AUC, may be determined with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% confidence.

Other related performance measures include positive and negative likelihood ratios [positive LR=sensitivity/(1−specificity); negative LR=(1−sensitivity)/specificity]. Such measures can also be used to gauge test performance according to the methods of the invention.

### Classification

Biosignature according to the invention can be used to classify a sample. Techniques for discriminate analysis are known to those of skill in the art. For example, a sample can be classified as, or predicted to be, a responder or non-responder to a given treatment for a given disease or disorder. Many statistical classification techniques are known to those of skill in the art. In supervised learning approaches, a group of samples from two or more groups are analyzed with a statistical classification method. One or more biomarkers, e.g., a panel of biomarkers that forms a biosignature, can be discovered that can be used to build a classifier that differentiates between the two or more groups. A new sample can then be analyzed so that the classifier can associate the new with one of the two or more groups. Commonly used supervised classifiers include without limitation the neural network (multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with the invention include quadratic classifiers, k-nearest neighbor, boosting, decision trees, random forests, neural networks, pattern recognition, Bayesian networks and Hidden Markov models. One of skill will appreciate that these or other classifiers, including modifications or improvements of those disclosed herein or known in the art, are contemplated within the scope of the invention.

Multivariate models that can be used to evaluate a biosignature comprising a presence or level of one or more biomarker include the following:

Linear Discriminant Analysis (LDA)

LDA is a well understood classification method that performs well for cases where predictors follow a generally normal distribution. The method can serve as a benchmark for more complex methods.

Diagonal Linear Discriminant Analysis (DLDA)

DLDA is version of discriminant analysis which assumes that predictors are independent, an assumption that may not hold true. However, when training data sets are too small to properly estimate covariances between predictors, well-fit DLDA model may consistently outperform more complex models.

Shrunken Centroids Discriminant Analysis (SCDA)

This method is commonly known within the mRNA micorarray community as “PAM” (prediction analysis for microarrays). The method is similar to other for discriminate analysis methods but uses more robust (stabilized) estimates of variance.

Support Vector Machines (SVM)

SVMs are a popular variety of machine learning. SVMs often outperforming traditional statistical methods when predictors are not easily transformed to a multivariate normal distribution. The final SVM model can be expressed in much the same way as an LDA model.

Tree-Based Gradient Boosting (GBM)

This method generates binary decision trees, using “boosting” to combine weakly performing trees in a weighted fashion to form a stronger ensemble.

Lasso (Lasso)

This approach fits a logistic regression model using “lasso” penalized maximum likelihood method. This approach tends to pick one representative marker from a set of highly correlated markers, returning zero values for coefficients of the remaining markers.

A classifier's performance can be estimated using a “training” set of sample to build a classifier and an independent “test” set of samples to test the model. Other techniques can be used in the art to estimate predictive performance, such as cross-validation methods. One round of cross-validation involves partitioning a sample of data into complementary subsets, performing the analysis on one subset (the training set), and validating the analysis on the other subset (the validation set or testing set). To reduce variability, multiple rounds of cross-validation can be performed using different partitions, and the validation results are averaged over the rounds. Common types of cross-validation include the following:

K-Fold Cross-Validation

The sample group is partitioned into k-partitions. One partition is used as the test set and the remainder are used as the training set. The process is repeated k times (or k folds) using each of the partitions once as the test set. The performance of the classifier model is averaged over the iterations. 10-fold cross validation is common though other numbers can be selected depending on sample size, computational resources, and the like.

2-Fold Cross-Validation

This is the simplest version of k-fold validation wherein the data is split into two equal size groups and each group is used for alternate rounds of training and testing.

Leave-One-Out Cross-Validation

In this approach, a single sample is withdrawn from the cohort for testing and the rest of the samples are used for training. If each sample is used once as the test sample, this approach is a form of k-folds cross validation where the number of iterations equals the number of samples.

Repeated Random Sub-Sampling Validation

In this approach, random subsets are drawn for the training and test set for each round of testing. As a result, each sample may not be used for both testing and training over the course of validation.

Classification using supervised methods is generally performed by the following methodology:

In order to solve a given problem of supervised learning (e.g. learning to distinguish between two biological states) one generally considers various steps:

1. Gather a training set. These can include, for example, samples that are from a subject with or without a disease or disorder, subjects that are known to respond or not respond to a treatment, subjects whose disease progresses or does not progress, etc. The training samples are used to “train” the classifier.

2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality; but should be large enough to accurately predict the output. The features might include a set of biomarkers such as those described herein.

3. Determine the structure of the learned function and corresponding learning algorithm. A learning algorithm is chosen, e.g., artificial neural networks, decision trees, Bayes classifiers or support vector machines. The learning algorithm is used to build the classifier.

4. Build the classifier. The learning algorithm is run the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier is determined as described above, it can be used to classify a sample, e.g., that of a subject who is being analyzed by the methods of the invention. As an example, a classifier can be built using data for levels of circulating biomarkers of interest in reference subjects with and without a disease as the training and test sets. Circulating biomarker levels found in a sample from a test subject are assessed and the classifier is used to classify the subject as with or without the disease. As another example, a classifier can be built using data for levels of vesicle biomarkers of interest in reference subjects that have been found to respond or not respond to certain diseases as the training and test sets. The vesicle biomarker levels found in a sample from a test subject are assessed and the classifier is used to classify the subject as with or without the disease.

Unsupervised learning approaches can also be used with the invention. Clustering is an unsupervised learning approach wherein a clustering algorithm correlates a series of samples without the use the labels. The most similar samples are sorted into “clusters.” A new sample could be sorted into a cluster and thereby classified with other members that it most closely associates. Many clustering algorithms well known to those of skill in the art can be used with the invention, such as hierarchical clustering.

### Biosignatures

A biosignature can be obtained according to the invention by assessing a vesicle population, including surface and payload vesicle associated biomarkers, and/or circulating biomarkers including microRNA and protein. A biosignature derived from a subject can be used to characterize a phenotype of the subject. A biosignature can further include the level of one or more additional biomarkers, e.g., circulating biomarkers or biomarkers associated with a vesicle of interest. A biosignature of a vesicle of interest can include particular antigens or biomarkers that are present on the vesicle. The biosignature can also include one or more antigens or biomarkers that are carried as payload within the vesicle, including the microRNA under examination. The biosignature can comprise a combination of one or more antigens or biomarkers that are present on the vesicle with one or more biomarkers that are detected in the vesicle. The biosignature can further comprise other information about a vesicle aside from its biomarkers. Such information can include vesicle size, circulating half-life, metabolic half-life, and specific activity in vivo or in vitro. The biosignature can comprise the biomarkers or other characteristics used to build a classifier.

In some embodiments, the microRNA is detected directly in a biological sample. For example, RNA in a bodily fluid can be isolated using commercially available kits such as mirVana kits (Applied Biosystems/Ambion, Austin, Tex.), MagMAX™ RNA Isolation Kit (Applied Biosystems/Ambion, Austin, Tex.), and QIAzol Lysis Reagent and RNeasy Midi Kit (Qiagen Inc., Valencia Calif.). Particular species of microRNAs can be determined using array or PCR techniques as described below.

In some embodiments, the microRNA payload with vesicles is assessed in order to characterize a phenotype. The vesicles can be purified or concentrated prior to determining the biosignature. For example, a cell-of-origin specific vesicle can be isolated and its biosignature determined. Alternatively, the biosignature of the vesicle can be directly assayed from a sample, without prior purification or concentration. The biosignature of the invention can be used to determine a diagnosis, prognosis, or theranosis of a disease or condition or similar measures described herein. A biosignature can also be used to determine treatment efficacy, stage of a disease or condition, or progression of a disease or condition, or responder/non-responder status. Furthermore, a biosignature may be used to determine a physiological state, such as pregnancy.

A characteristic of a vesicle in and of itself can be assessed to determine a biosignature. The characteristic can be used to diagnose, detect or determine a disease stage or progression, the therapeutic implications of a disease or condition, or characterize a physiological state. Such characteristics include without limitation the level or amount of vesicles, vesicle size, temporal evaluation of the variation in vesicle half-life, circulating vesicle half-life, metabolic half-life of a vesicle, or activity of a vesicle.

Biomarkers that can be included in a biosignature include one or more proteins or peptides (e.g., providing a protein signature), nucleic acids (e.g. RNA signature as described, or a DNA signature), lipids (e.g. lipid signature), or combinations thereof. In some embodiments, the biosignature can also comprise the type or amount of drug or drug metabolite present in a vesicle, (e.g., providing a drug signature), as such drug may be taken by a subject from which the biological sample is obtained, resulting in a vesicle carrying the drug or metabolites of the drug.

A biosignature can also include an expression level, presence, absence, mutation, variant, copy number variation, truncation, duplication, modification, or molecular association of one or more biomarkers. A genetic variant, or nucleotide variant, refers to changes or alterations to a gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant may occur in transcriptional regulatory regions, untranslated regions of mRNA, exons, introns, or exon/intron junctions. The genetic variant may or may not result in stop codons, frame shifts, deletions of amino acids, altered gene transcript splice forms or altered amino acid sequence.

In an embodiment, nucleic acid biomarkers, including nucleic acid payload within a vesicle, is assessed for nucleotide variants. The nucleic acid biomarker may comprise one or more RNA species, e.g., mRNA, miRNA, snoRNA, snRNA, rRNAs, tRNAs, siRNA, hnRNA, shRNA, enhancer RNA (eRNA), or a combination thereof. Similarly, DNA payload can be assessed to form a DNA signature.

An RNA signature or DNA signature can also include a mutational, epigenetic modification, or genetic variant analysis of the RNA or DNA present in the vesicle. Epigenetic modifications include patterns of DNA methylation. See, e.g., Lesche R. and Eckhardt F., DNA methylation markers: a versatile diagnostic tool for routine clinical use. Curr Opin Mol Ther. 2007 Jun. 9(3):222-30, which is incorporated herein by reference in its entirety. Thus, a biomarker can be the methylation status of a segment of DNA.

A biosignature can comprise one or more miRNA signatures combined with one or more additional signatures including, but not limited to, an mRNA signature, DNA signature, protein signature, peptide signature, antigen signature, or any combination thereof. For example, the biosignature can comprise one or more miRNA biomarkers with one or more DNA biomarkers, one or more mRNA biomarkers, one or more snoRNA biomarkers, one or more protein biomarkers, one or more peptide biomarkers, one or more antigen biomarkers, one or more antigen biomarkers, one or more lipid biomarkers, or any combination thereof.

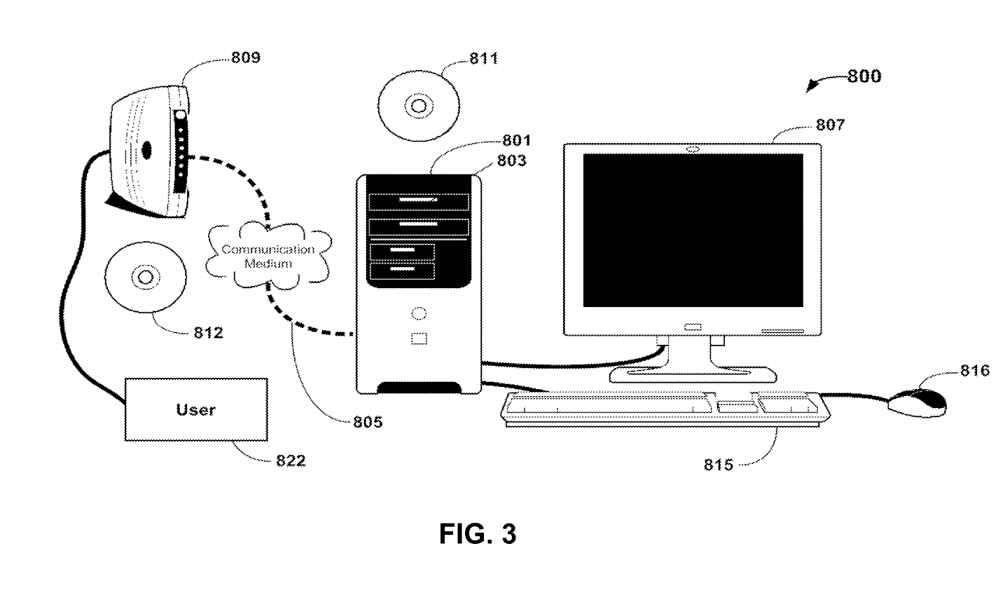

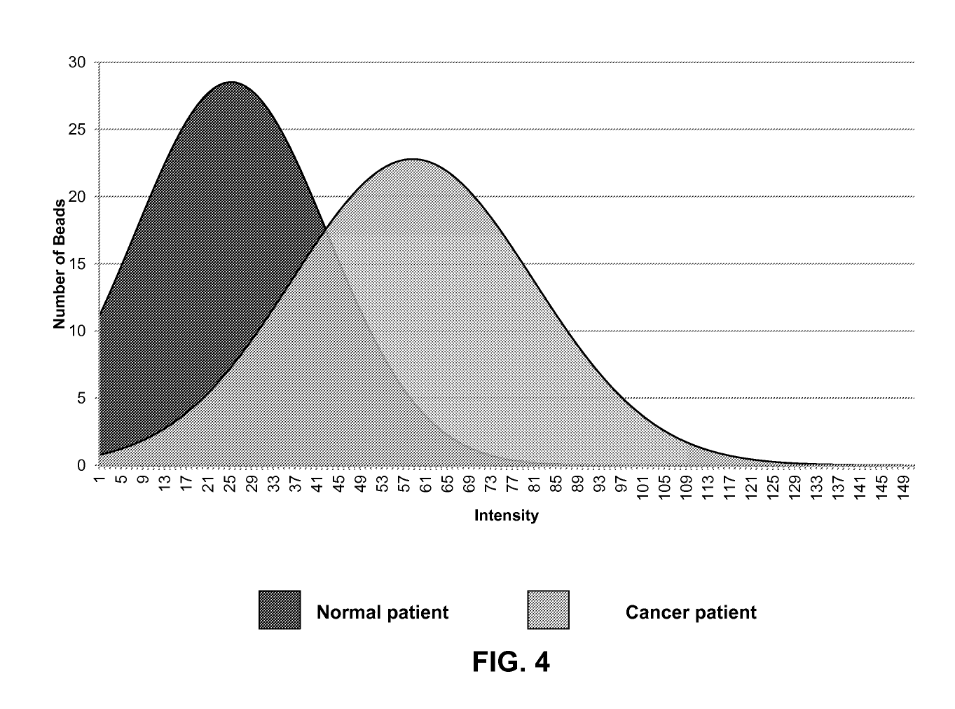

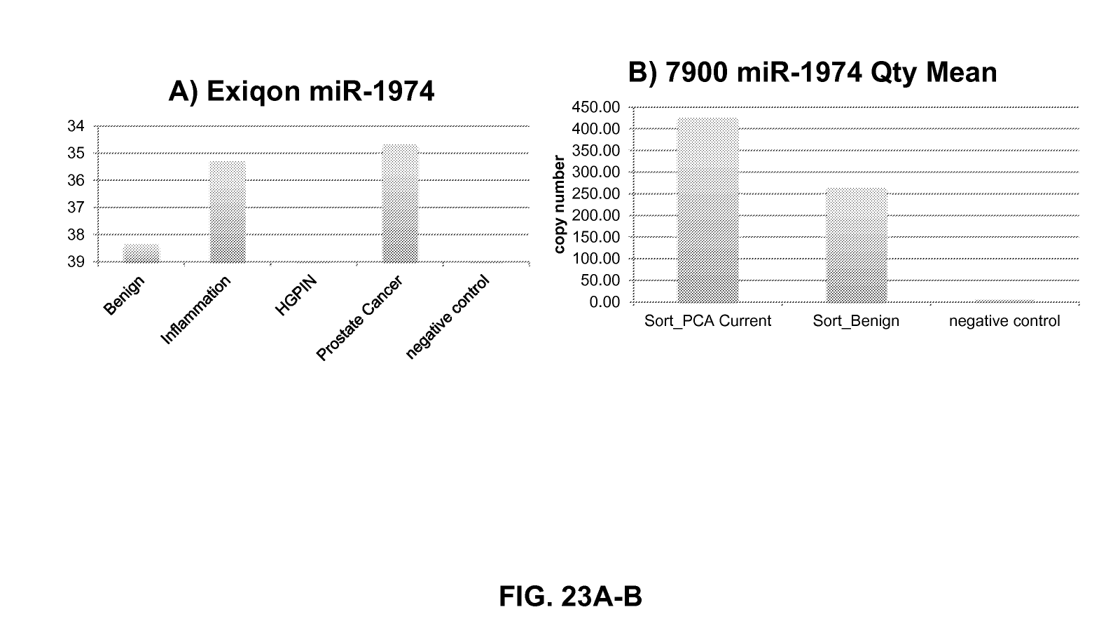

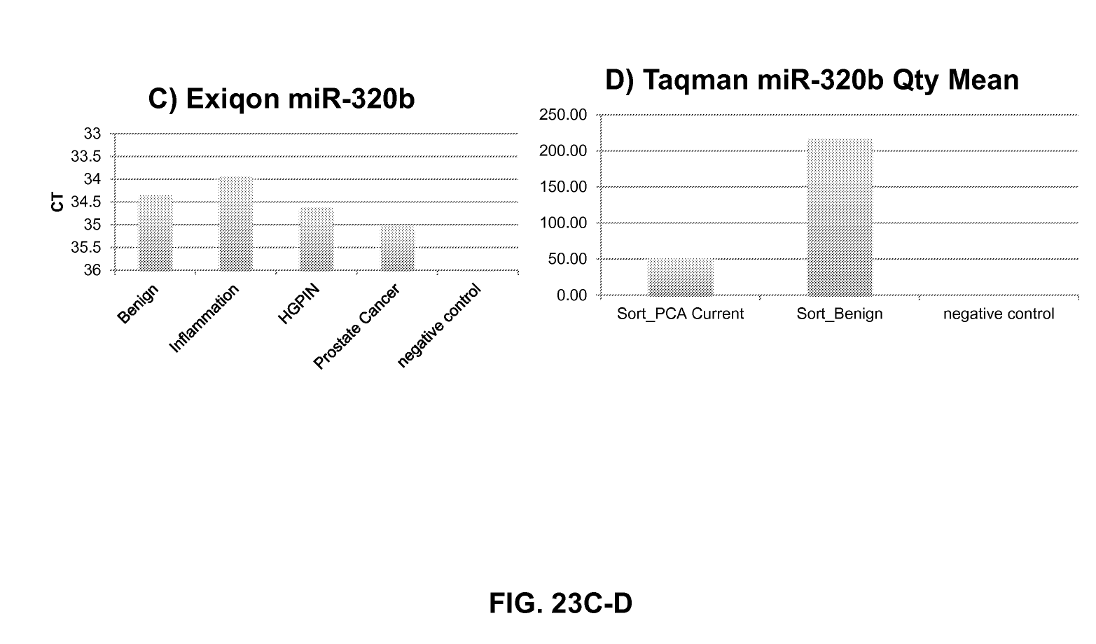

A biosignature can comprise a combination of one or more antigens or binding agents (such as ability to bind one or more binding agents), such as listed in FIGS. 1 and 2, respectively, of International Patent Application Serial No. PCT/US2011/031479, entitled “Circulating Biomarkers for Disease” and filed Apr. 6, 2011, which application is incorporated by reference in its entirety herein, or those described elsewhere herein. The biosignature can further comprise one or more other biomarkers, such as, but not limited to, miRNA, DNA (e.g. single stranded DNA, complementary DNA, or noncoding DNA), or mRNA. The biosignature of a vesicle can comprise a combination of one or more antigens, such as shown in FIG. 1 of International Patent Application Serial No. PCT/US2011/031479, one or more binding agents, such as shown in FIG. 2 of International Patent Application Serial No. PCT/US2011/031479, and one or more biomarkers for a condition or disease, such as listed in FIGS. 3-60 of International Patent Application Serial No. PCT/US2011/031479. The biosignature can comprise one or more biomarkers, for example miRNA, with one or more antigens specific for a cancer cell (for example, as shown in FIG. 1 of International Patent Application Serial No. PCT/US2011/031479).

In some embodiments, a vesicle used in the subject methods has a biosignature that is specific to the cell-of-origin and is used to derive disease-specific or biological state specific diagnostic, prognostic or therapy-related biosignatures representative of the cell-of-origin. In other embodiments, a vesicle has a biosignature that is specific to a given disease or physiological condition that is different from the biosignature of the cell-of-origin for use in the diagnosis, prognosis, staging, therapy-related determinations or physiological state characterization. Biosignatures can also comprise a combination of cell-of-origin specific and non-specific vesicles.

Biosignatures can be used to evaluate diagnostic criteria such as presence of disease, disease staging, disease monitoring, disease stratification, or surveillance for detection, metastasis or recurrence or progression of disease. A biosignature can also be used clinically in making decisions concerning treatment modalities including therapeutic intervention. A biosignature can further be used clinically to make treatment decisions, including whether to perform surgery or what treatment standards should be used along with surgery (e.g., either pre-surgery or post-surgery). As an illustrative example, a biosignature of circulating biomarkers that indicates an aggressive form of cancer may call for a more aggressive surgical procedure and/or more aggressive therapeutic regimen to treat the patient.

A biosignature can be used in therapy related diagnostics to provide tests useful to diagnose a disease or choose the correct treatment regimen, such as provide a theranosis. Theranostics includes diagnostic testing that provides the ability to affect therapy or treatment of a diseased state. Theranostics testing provides a theranosis in a similar manner that diagnostics or prognostic testing provides a diagnosis or prognosis, respectively. As used herein, theranostics encompasses any desired form of therapy related testing, including predictive medicine, personalized medicine, integrated medicine, pharmacodiagnostics and Dx/Rx partnering. Therapy related tests can be used to predict and assess drug response in individual subjects, i.e., to provide personalized medicine. Predicting a drug response can be determining whether a subject is a likely responder or a likely non-responder to a candidate therapeutic agent, e.g., before the subject has been exposed or otherwise treated with the treatment. Assessing a drug response can be monitoring a response to a drug, e.g., monitoring the subject's improvement or lack thereof over a time course after initiating the treatment. Therapy related tests are useful to select a subject for treatment who is particularly likely to benefit from the treatment or to provide an early and objective indication of treatment efficacy in an individual subject. Thus, a biosignature as disclosed herein may indicate that treatment should be altered to select a more promising treatment, thereby avoiding the great expense of delaying beneficial treatment and avoiding the financial and morbidity costs of administering an ineffective drug(s).

Therapy related diagnostics are also useful in clinical diagnosis and management of a variety of diseases and disorders, which include, but are not limited to cardiovascular disease, cancer, infectious diseases, sepsis, neurological diseases, central nervous system related diseases, endovascular related diseases, and autoimmune related diseases. Therapy related diagnostics also aid in the prediction of drug toxicity, drug resistance or drug response. Therapy related tests may be developed in any suitable diagnostic testing format, which include, but are not limited to, e.g., immunohistochemical tests, clinical chemistry, immunoassay, cell-based technologies, nucleic acid tests or body imaging methods. Therapy related tests can further include but are not limited to, testing that aids in the determination of therapy, testing that monitors for therapeutic toxicity, or response to therapy testing. Thus, a biosignature can be used to predict or monitor a subject's response to a treatment. A biosignature can be determined at different time points for a subject after initiating, removing, or altering a particular treatment.

In some embodiments, a determination or prediction as to whether a subject is responding to a treatment is made based on a change in the amount of one or more components of a biosignature (i.e., the microRNA, vesicles and/or biomarkers of interest), an amount of one or more components of a particular biosignature, or the biosignature detected for the components. In another embodiment, a subject's condition is monitored by determining a biosignature at different time points. The progression, regression, or recurrence of a condition is determined. Response to therapy can also be measured over a time course. Thus, the invention provides a method of monitoring a status of a disease or other medical condition in a subject, comprising isolating or detecting a biosignature from a biological sample from the subject, detecting the overall amount of the components of a particular biosignature, or detecting the biosignature of one or more components (such as the presence, absence, or expression level of a biomarker). The biosignatures are used to monitor the status of the disease or condition.

One or more novel biosignatures of a vesicle can also be identified. For example, one or more vesicles can be isolated from a subject that responds to a drug treatment or treatment regimen and compared to a reference, such as another subject that does not respond to the drug treatment or treatment regimen. Differences between the biosignatures can be determined and used to identify other subjects as responders or non-responders to a particular drug or treatment regimen.

In some embodiments, a biosignature is used to determine whether a particular disease or condition is resistant to a drug. If a subject is drug resistant, a physician need not waste valuable time with such drug treatment. To obtain early validation of a drug choice or treatment regimen, a biosignature is determined for a sample obtained from a subject. The biosignature is used to assess whether the particular subject's disease has the biomarker associated with drug resistance. Such a determination enables doctors to devote critical time as well as the patient's financial resources to effective treatments.

Moreover, biosignature may be used to assess whether a subject is afflicted with disease, is at risk for developing disease or to assess the stage or progression of the disease. For example, a biosignature can be used to assess whether a subject has prostate cancer, colon cancer, or other cancer as described herein. Futhermore, a biosignature can be used to determine a stage of a disease or condition, such as colon cancer.

Furthermore, determining the amount of vesicles, such a heterogeneous population of vesicles, and the amount of one or more homogeneous population of vesicles, such as a population of vesicles with the same biosignature, can be used to characterize a phenotype. For example, determination of the total amount of vesicles in a sample (i.e. not cell-type specific) and determining the presence of one or more different cell-of-origin specific vesicles can be used to characterize a phenotype. Threshold values, or reference values or amounts can be determined based on comparisons of normal subjects and subjects with the phenotype of interest, as further described below, and criteria based on the threshold or reference values determined. The different criteria can be used to characterize a phenotype.

One criterion can be based on the amount of a heterogeneous population of vesicles in a sample. In one embodiment, general vesicle markers, such as CD9, CD81, and CD63 can be used to determine the amount of vesicles in a sample. The expression level of CD9, CD81, CD63, or a combination thereof can be detected and if the level is greater than a threshold level, the criterion is met. In another embodiment, the criterion is met if if level of CD9, CD81, CD63, or a combination thereof is lower than a threshold value or reference value. In another embodiment, the criterion can be based on whether the amount of vesicles is higher than a threshold or reference value. Another criterion can be based on the amount of vesicles with a specific biosignature. If the amount of vesicles with the specific biosignature is lower than a threshold or reference value, the criterion is met. In another embodiment, if the amount of vesicles with the specific biosignature is higher than a threshold or reference value, the criterion is met. A criterion can also be based on the amount of vesicles derived from a particular cell type. If the amount is lower than a threshold or reference value, the criterion is met. In another embodiment, if the amount is higher than a threshold value, the criterion is met.

In a non-limiting example, consider that vesicles from prostate cells are determined by detecting the biomarker PCSA or PSCA, and that a criterion is met if the level of detected PCSA or PSCA is greater than a threshold level. The threshold can be the level of the same markers in a sample from a control cell line or control subject. Another criterion can be based on whether the amount of vesicles derived from a cancer cell or comprising one or more cancer specific biomarkers. For example, the biomarkers B7H3, EpCam, or both, can be determined and a criterion met if the level of detected B7H3 and/or EpCam is greater than a threshold level or within a pre-determined range. If the amount is lower, or higher, than a threshold or reference value, the criterion is met. A criterion can also be the reliability of the result, such as meeting a quality control measure or value. A detected amount of B7H3 and/or EpCam in a test sample that is above the amount of these markers in a control sample may indicate the presence of a cancer in the test sample.

As described, analysis of multiple markers can be combined to assess whether a criterion is met. In an illustrative example, a biosignature is used to assess whether a subject has prostate cancer by detecting one or more of the general vesicle markers CD9, CD63 and CD81; one or more prostate epithelial markers including PCSA or PSMA; and one or more cancer markers such as B7H3 and/or EpCam. Higher levels of the markers in a sample from a subject than in a control individual without prostate cancer indicates the presence of the prostate cancer in the subject. In some embodiments, the multiple markers are assessed in a multiplex fashion.

One of skill will understand that such rules based on meeting criterion as described can be applied to any appropriate biomarker. For example, the criterion can be applied to vesicle characteristics such as amount of vesicles present, amount of vesicles with a particular biosignature present, amount of vesicle payload biomarkers present, amount of microRNA or other circulating biomarkers present, and the like. The ratios of appropriate biomarkers can be determined. As illustrative examples, the criterion could be a ratio of an vesicle surface protein to another vesicle surface protein, a ratio of an vesicle surface protein to a microRNA, a ratio of one vesicle population to another vesicle population, a ratio of one circulating biomarker to another circulating biomarker, etc.

A phenotype for a subject can be characterized based on meeting any number of useful criteria. In some embodiments, at least one criterion is used for each biomarker. In some embodiments, at least 1, 2, 3, 4, 5, 6, 7, 8, 9, 10, 15, 20, 30, 40, 50, 60, 70, 80, 90 or at least 100 criteria are used. For example, for the characterizing of a cancer, a number of different criteria can be used when the subject is diagnosed with a cancer: 1) if the amount of microRNA in a sample from a subject is higher than a reference value; 2) if the amount of a microRNA within cell type specific vesicles (i.e. vesicles derived from a specific tissue or organ) is higher than a reference value; or 3) if the amount of microRNA within vesicles with one or more cancer specific biomarkers is higher than a reference value. Similar rules can apply if the amount of microRNA is less than or the same as the reference. The method can further include a quality control measure, such that the results are provided for the subject if the samples meet the quality control measure. In some embodiments, if the criteria are met but the quality control is questionable, the subject is reassessed.

In other embodiments, a single measure is determined for assessment of multiple biomarkers, and the measure is compared to a reference. For illustration, a test for prostate cancer might comprise multiplying the level of PSA against the level of miR-141 in a blood sample. The criterion is met if the product of the levels is above a threshold, indicating the presense of the cancer. As another illustration, a number of binding agents to general vesicle markers can carry the same label, e.g., the same fluorophore. The level of the detected label can be compared to a threshold.

Criterion can be applied to multiple types of biomarkers in addition to multiple biomarkers of the same type. For example, the levels of one or more circulating biomarkers (e.g., RNA, DNA, peptides), vesicles, mutations, etc, can be compared to a reference. Different components of a biosignature can have different criteria. As a non-limiting example, a biosignature used to diagnose a cancer can include overexpression of one miR species as compared to a reference and underexpression of a vesicle surface antigen as compared to another reference.

A biosignature can be determined by comparing the amount of vesicles, the structure of a vesicle, or any other informative characteristic of a vesicle. Vesicle structure can be assessed using transmission electron microscopy, see for example, Hansen et al., Journal of Biomechanics 31, Supplement 1: 134-134(1) (1998), or scanning electron microscopy. Various combinations of methods and techniques or analyzing one or more vesicles can be used to determine a phenotype for a subject.

A biosignature can include without limitation the presence or absence, copy number, expression level, or activity level of a biomarker. Other useful components of a biosignature include the presence of a mutation (e.g., mutations which affect activity of a transcription or translation product, such as substitution, deletion, or insertion mutations), variant, or post-translation modification of a biomarker. Post-translational modification of a protein biomarker include without limitation acylation, acetylation, phosphorylation, ubiquitination, deacetylation, alkylation, methylation, amidation, biotinylation, gamma-carboxylation, glutamylation, glycosylation, glycyation, hydroxylation, covalent attachment of heme moiety, iodination, isoprenylation, lipoylation, prenylation, GPI anchor formation, myristoylation, farnesylation, geranylgeranylation, covalent attachment of nucleotides or derivatives thereof, ADP-ribosylation, flavin attachment, oxidation, palmitoylation, pegylation, covalent attachment of phosphatidylinositol, phosphopantetheinylation, polysialylation, pyroglutamate formation, racemization of proline by prolyl isomerase, tRNA-mediation addition of amino acids such as arginylation, sulfation, the addition of a sulfate group to a tyrosine, or selenoylation of the biomarker.

The methods described herein can be used to identify a biosignature that is associated with a disease, condition or physiological state. The biosignature can also be used to determine if a subject is afflicted with cancer or is at risk for developing cancer. A subject at risk of developing cancer can include those who may be predisposed or who have pre-symptomatic early stage disease.

A biosignature can also be used to provide a diagnostic or theranostic determination for other diseases including but not limited to autoimmune diseases, inflammatory bowel diseases, cardiovascular disease, neurological disorders such as Alzheimer's disease, Parkinson's disease, Multiple Sclerosis, sepsis or pancreatitis or any disease, conditions or symptoms listed in FIGS. 3-58 of International Patent Application Serial No. PCT/US2011/031479, entitled “Circulating Biomarkers for Disease” and filed Apr. 6, 2011, which application is incorporated by reference in its entirety herein.

The biosignature can also be used to identify a given pregnancy state from the peripheral blood, umbilical cord blood, or amniotic fluid (e.g. miRNA signature specific to Downs Syndrome) or adverse pregnancy outcome such as pre-eclampsia, pre-term birth, premature rupture of membranes, intrauterine growth restriction or recurrent pregnancy loss. The biosignature can also be used to indicate the health of the mother, the fetus at all developmental stages, the pre-implantation embryo or a newborn.

A biosignature can be used for pre-symptomatic diagnosis. Furthermore, the biosignature can be used to detect disease, determine disease stage or progression, determine the recurrence of disease, identify treatment protocols, determine efficacy of treatment protocols or evaluate the physiological status of individuals related to age and environmental exposure.

Monitoring a biosignature of a vesicle can also be used to identify toxic exposures in a subject including, but not limited to, situations of early exposure or exposure to an unknown or unidentified toxic agent. Without being bound by any one specific theory for mechanism of action, vesicles can shed from damaged cells and in the process compartmentalize specific contents of the cell including both membrane components and engulfed cytoplasmic contents. Cells exposed to toxic agents/chemicals may increase vesicle shedding to expel toxic agents or metabolites thereof, thus resulting in increased vesicle levels. Thus, monitoring vesicle levels, vesicle biosignature, or both, allows assessment of an individual's response to potential toxic agent(s).

A vesicle and/or other biomarkers of the invention can be used to identify states of drug-induced toxicity or the organ injured, by detecting one or more specific antigen, binding agent, biomarker, or any combination thereof. The level of vesicles, changes in the biosignature of a vesicle, or both, can be used to monitor an individual for acute, chronic, or occupational exposures to any number of toxic agents including, but not limited to, drugs, antibiotics, industrial chemicals, toxic antibiotic metabolites, herbs, household chemicals, and chemicals produced by other organisms, either naturally occurring or synthetic in nature. In addition, a biosignature can be used to identify conditions or diseases, including cancers of unknown origin, also known as cancers of unknown primary (CUP).

A vesicle may be isolated from a biological sample as previously described to arrive at a heterogeneous population of vesicles. The heterogeneous population of vesicles can then be contacted with substrates coated with specific binding agents designed to rule out or identify antigen specific characteristics of the vesicle population that are specific to a given cell-of-origin. Further, as described above, the biosignature of a vesicle can correlate with the cancerous state of cells. Compounds that inhibit cancer in a subject may cause a change, e.g., a change in biosignature of a vesicle, which can be monitored by serial isolation of vesicles over time and treatment course. The level of vesicles or changes in the level of vesicles with a specific biosignature can be monitored.

In an aspect, characterizing a phenotype of a subject comprises a method of determining whether the subject is likely to respond or not respond to a therapy. The methods of the invention also include determining new biosignatures useful in predicting whether the subject is likely to respond or not. One or more subjects that respond to a therapy (responders) and one or more subjects that do not respond to the same therapy (non-responders) can have their vesicles interrogated. Interrogation can be performed to identify vesicle biosignatures that classify a subject as a responder or non-responder to the treatment of interest. In some aspects, the presence, quantity, and payload of a vesicle are assayed. The payload of a vesicle includes, for example, internal proteins, nucleic acids such as miRNA, lipids or carbohydrates.

The presence or absence of a biosignature in responders but not in the non-responders can be used for theranosis. A sample from responders may be analyzed for one or more of the following: amount of vesicles, amount of a unique subset or species of vesicles, biomarkers in such vesicles, biosignature of such vesicles, etc. In one instance, vesicles such as microvesicles or exosomes from responders and non-responders are analyzed for the presence and/or quantity of one or more miRNAs, such as miRNA 122, miR-548c-5p, miR-362-3p, miR-422a, miR-597, miR-429, miR-200a, and/or miR-200b. A difference in biosignatures between responders and non-responders can be used for theranosis. In another embodiment, vesicles are obtained from subjects having a disease or condition. Vesicles are also obtained from subjects free of such disease or condition. The vesicles from both groups of subjects are assayed for unique biosignatures that are associated with all subjects in that group but not in subjects from the other group. Such biosignatures or biomarkers can then used as a diagnostic for the presence or absence of the condition or disease, or to classify the subject as belonging on one of the groups (those with/without disease, aggressive/non-aggressive disease, responder/non-responder, etc).

In an aspect, characterizing a phenotype of a subject comprises a method of staging a disease. The methods of the invention also include determining new biosignatures useful in staging. In an illustrative example, vesicles are assayed from patients having a stage I cancer and patients having stage II or stage III of the same cancer. In some embodiments, vesicles are assayed in patients with metastatic disease. A difference in biosignatures or biomarkers between vesicles from each group of patient is identified (e.g., vesicles from stage III cancer may have an increased expression of one or more genes or miRNA's), thereby identifying a biosignature or biomarker that distinguishes different stages of a disease. Such biosignature can then be used to stage patients having the disease.

In some instances, a biosignature is determined by assaying vesicles from a subject over a period of time, e.g., daily, semiweekly, weekly, biweekly, semimonthly, monthly, bimonthly, semiquarterly, quarterly, semiyearly, biyearly or yearly. For example, the biosignatures in patients on a given therapy can be monitored over time to detect signatures indicative of responders or non-responders for the therapy. Similarly, patients with differing stages of disease or in differing stages of a clinical trial have a biosignature interrogated over time. The payload or physical attributes of the vesicles in each point in time can be compared. A temporal pattern can thus form a biosignature that can then be used for theranosis, diagnosis, prognosis, disease stratification, treatment monitoring, disease monitoring or making a prediction of responder/non-responder status. As an illustrative example only, an increasing amount of a biomarker (e.g., miR 122) in vesicles over a time course is associated with metastatic cancer, as opposed to a stagnant amounts of the biomarker in vesicles over the time course that are associated with non-metastatic cancer. A time course may last over at least 1 week, 2 weeks, 3 weeks, 4 weeks, 1 month, 6 weeks, 8 weeks, 2 months, 10 weeks, 12 weeks, 3 months, 4 months, 5 months, 6 months, 7 months, 8 months, 9 months, 10 months, 11 months, 12 months, one year, 18 months, 2 years, or at least 3 years.

The level of vesicles, level of vesicles with a specific biosignature, or a biosignature of a vesicle can also be used to assess the efficacy of a therapy for a condition. For example, the level of vesicles, level of vesicles with a specific biosignature, or a biosignature of a vesicle can be used to assess the efficacy of a cancer treatment, e.g., chemotherapy, radiation therapy, surgery, or any other therapeutic approach useful for inhibiting cancer in a subject. In addition, a biosignature can be used in a screening assay to identify candidate or test compounds or agents (e.g., proteins, peptides, peptidomimetics, peptoids, small molecules or other drugs) that have a modulatory effect on the biosignature of a vesicle. Compounds identified via such screening assays may be useful, for example, for modulating, e.g., inhibiting, ameliorating, treating, or preventing conditions or diseases.

For example, a biosignature for a vesicle can be obtained from a patient who is undergoing successful treatment for a particular cancer. Cells from a cancer patient not being treated with the same drug can be cultured and vesicles from the cultures obtained for determining biosignatures. The cells can be treated with test compounds and the biosignature of the vesicles from the cultures can be compared to the biosignature of the vesicles obtained from the patient undergoing successful treatment. The test compounds that results in biosignatures that are similar to those of the patient undergoing successful treatment can be selected for further studies.

The biosignature of a vesicle can also be used to monitor the influence of an agent (e.g., drug compounds) on the biosignature in clinical trials. Monitoring the level of vesicles, changes in the biosignature of a vesicle, or both, can also be used in a method of assessing the efficacy of a test compound, such as a test compound for inhibiting cancer cells.

In addition to diagnosing or confirming the presence of or risk for developing a disease, condition or a syndrome, the methods and compositions disclosed herein also provide a system for optimizing the treatment of a subject having such a disease, condition or syndrome. The level of vesicles, the biosignature of a vesicle, or both, can also be used to determine the effectiveness of a particular therapeutic intervention (pharmaceutical or non-pharmaceutical) and to alter the intervention to 1) reduce the risk of developing adverse outcomes, 2) enhance the effectiveness of the intervention or 3) identify resistant states. Thus, in addition to diagnosing or confirming the presence of or risk for developing a disease, condition or a syndrome, the methods and compositions disclosed herein also provide a system for optimizing the treatment of a subject having such a disease, condition or syndrome. For example, a therapy-related approach to treating a disease, condition or syndrome by integrating diagnostics and therapeutics to improve the real-time treatment of a subject can be determined by identifying the biosignature of a vesicle.

Tests that identify the level of vesicles, the biosignature of a vesicle, or both, can be used to identify which patients are most suited to a particular therapy, and provide feedback on how well a drug is working, so as to optimize treatment regimens. For example, in pregnancy-induced hypertension and associated conditions, therapy-related diagnostics can flexibly monitor changes in important parameters (e.g., cytokine and/or growth factor levels) over time, to optimize treatment.

Within the clinical trial setting of investigational agents as defined by the FDA, MDA, EMA, USDA, and EMEA, therapy-related diagnostics as determined by a biosignature disclosed herein, can provide key information to optimize trial design, monitor efficacy, and enhance drug safety. For instance, for trial design, therapy-related diagnostics can be used for patient stratification, determination of patient eligibility (inclusion/exclusion), creation of homogeneous treatment groups, and selection of patient samples that are optimized to a matched case control cohort. Such therapy-related diagnostic can therefore provide the means for patient efficacy enrichment, thereby minimizing the number of individuals needed for trial recruitment. For example, for efficacy, therapy-related diagnostics are useful for monitoring therapy and assessing efficacy criteria. Alternatively, for safety, therapy-related diagnostics can be used to prevent adverse drug reactions or avoid medication error and monitor compliance with the therapeutic regimen.

In some embodiments, the invention provides a method of identifying responder and non-responders to a treatment undergoing clinical trials, comprising detecting biosignatures comprising circulating biomarkers in subjects enrolled in the clinical trial, and identifying biosignatures that distinguish between responders and non-responders. In a further embodiment, the biosignatures are measured in a drug naive subject and used to predict whether the subject will be a responder or non-responder. The prediction can be based upon whether the biosignatures of the drug naive subject correlate more closely with the clinical trial subjects identified as responders, thereby predicting that the drug naive subject will be a responder. Conversely, if the biosignatures of the drug naive subject correlate more closely with the clinical trial subjects identified as non-responders, the methods of the invention can predict that the drug naive subject will be a non-responder. The prediction can therefore be used to stratify potential responders and non-responders to the treatment. In some embodiments, the prediction is used to guide a course of treatment, e.g., by helping treating physicians decide whether to administer the drug. In some embodiments, the prediction is used to guide selection of patients for enrollment in further clinical trials. In a non-limiting example, biosignatures that predict responder/non-responder status in Phase II trials can be used to select patients for a Phase III trial, thereby increasing the likelihood of response in the Phase III patient population. One of skill will appreciate that the method can be adapted to identify biosignatures to stratify subjects on criteria other than responder/non-responder status. In one embodiment, the criterion is treatment safety. Therefore the method is followed as above to identify subjects who are likely or not to have adverse events to the treatment. In a non-limiting example, biosignatures that predict safety profile in Phase II trials can be used to select patients for a Phase III trial, thereby increasing the treatment safety profile in the Phase III patient population.

Therefore, the level of vesicles, the biosignature of a vesicle, or both, can be used to monitor drug efficacy, determine response or resistance to a given drug, or both, thereby enhancing drug safety. For example, in colon cancer, vesicles are typically shed from colon cancer cells and can be isolated from the peripheral blood and used to isolate one or more biomarkers e.g., KRAS mRNA which can then be sequenced to detect KRAS mutations. In the case of mRNA biomarkers, the mRNA can be reverse transcribed into cDNA and sequenced (e.g., by Sanger sequencing, pyrosequencing, NextGen sequencing, RT-PCR assays) to determine if there are mutations present that confer resistance to a drug (e.g., cetuximab or panitumimab). In another example, vesicles that are specifically shed from lung cancer cells are isolated from a biological sample and used to isolate a lung cancer biomarker, e.g., EGFR mRNA. The EGFR mRNA is processed to cDNA and sequenced to determine if there are EGFR mutations present that show resistance or response to specific drugs or treatments for lung cancer.

One or more biosignatures can be grouped so that information obtained about the set of biosignatures in a particular group provides a reasonable basis for making a clinically relevant decision, such as but not limited to a diagnosis, prognosis, or management of treatment, such as treatment selection.

As with most diagnostic markers, it is often desirable to use the fewest number of markers sufficient to make a correct medical judgment. This prevents a delay in treatment pending further analysis as well inappropriate use of time and resources.

Also disclosed herein are methods of conducting retrospective analysis on samples (e.g., serum and tissue biobanks) for the purpose of correlating qualitative and quantitative properties, such as biosignatures of vesicles, with clinical outcomes in terms of disease state, disease stage, progression, prognosis; therapeutic efficacy or selection; or physiological conditions. Furthermore, methods and compositions disclosed herein are used for conducting prospective analysis on a sample (e.g., serum and/or tissue collected from individuals in a clinical trial) for the purpose of correlating qualitative and quantitative biosignatures of vesicleswith clinical outcomes in terms of disease state, disease stage, progression, prognosis; therapeutic efficacy or selection; or physiological conditions can also be performed. As used herein, a biosignature for a vesicle can be used to identify a cell-of-origin specific vesicle. Furthermore, a biosignature can be determined based on a surface marker profile of a vesicle or contents of a vesicle.

The biosignatures used to characterize a phenotype according to the invention can comprise multiple components (e.g., microRNA, vesicles or other biomarkers) or characteristics (e.g., vesicle size or morphology). The biosignatures can comprise at least 2, 3, 4, 5, 6, 7, 8, 9, 10, 11, 12, 13, 14, 15, 16, 17, 18, 19, 20, 25, 30, 40, 50, 75, or 100 components or characteristics. A biosignature with more than one component or characteristic, such as at least 2, 3, 4, 5, 6, 7, 8, 9, 10, 11, 12, 13, 14, 15, 16, 17, 18, 19, 20, 25, 30, 40, 50, 75, or 100 components, may provide higher sensitivity and/or specificity in characterizing a phenotype. In some embodiments, assessing a plurality of components or characteristics provides increased sensitivity and/or specificity as compared to assessing fewer components or characteristics. On the other hand, it is often desirable to use the fewest number of components or characteristics sufficient to make a correct medical judgment. Fewer markers can avoid statistical overfitting of a classifier and can prevent a delay in treatment pending further analysis as well inappropriate use of time and resources. Thus, the methods of the invention comprise determining an optimal number of components or characteristics.

A biosignature according to the invention can be used to characterize a phenotype with a sensitivity, specificity, accuracy, or similar performance metric as described above. The biosignatures can also be used to build a classifier to classify a sample as belonging to a group, such as belonging to a group having a disease or not, a group having an aggressive disease or not, or a group of responders or non-responders. In one embodiment, a classifier is used to determine whether a subject has an aggressive or non-aggressive cancer. In the illustrative case of prostate cancer, this can help a physician to determine whether to watch the cancer, i.e., prescribe “watchful waiting,” or perform a prostatectomy. In another embodiment, a classifier is used to determine whether a breast cancer patient is likely to respond or not to tamoxifen, thereby helping the physician to determine whether or not to treat the patient with tamoxifen or another drug.

### Biomarkers

As described herein, the methods and compositions of the invention can be used in assays to detect the presence or level of one or more biomarker of interest. The biomarker can be any useful biomarker disclosed herein or known to those of skill in the art. In an embodiment, the biomarker comprises a protein or polypeptide. As used herein, “protein,” “polypeptide” and “peptide” are used interchangeably unless stated otherwise. The biomarker can be a nucleic acid, including DNA, RNA, and various subspecies of any thereof as disclosed herein or known in the art. The biomarker can comprise a lipid. The biomarker can comprise a carbohydrate. The biomarker can also be a complex, e.g., a complex comprising protein, nucleic acids, lipids and/or carbohydrates. In some embodiments, the biomarker comprises a microvesicle.

A biosignature comprising more than one biomarker can comprise one type of biomarker or multiple types of biomarkers. As a non-limiting example, a biosignature can comprise multiple proteins, multiple nucleic acids, multiple lipids, multiple carbohydrates, multiple biomarker complexes, multiple microvesicles, or a combination of any thereof. For example, the biosignature may comprise one or more microvesicle, one or more protein, and one or more microRNA, wherein the one or more protein and/or one or more microRNA is optionally in association with the microvesicle as a surface antigen and/or payload, as appropriate.

The biosignature can include the presence or absence, expression level, mutational state, genetic variant state, or any modification (such as epigenetic modification, or post-translation modification) of a biomarker disclosed herein (e.g., Tables 3, 4 or 5) or previously disclosed (e.g. any one or more biomarker listed in FIGS. 1, 3-60 of International Patent Application Serial No. PCT/US2011/031479, entitled “Circulating Biomarkers for Disease” and filed Apr. 6, 2011, which application is incorporated by reference in its entirety herein). One of skill will recognize that methods of the invention can be adapted to assess one or more biomarkers disclosed herein for a disease or condition different than a disease that is conventionally associated with a given biomarker. For example, one or more biomarkers disclosed herein for condition x may readily be utilized in obtaining a biosignature for a different condition y, based on the teachings of the instant disclosure and methods of the invention. The expression level of a biomarker can be compared to a control or reference, to determine the overexpression or underexpression (or upregulation or downregulation) of a biomarker in a sample. In some embodiments, the control or reference level comprises the amount of a same biomarker, such as a miRNA, in a control sample from a subject that does not have or exhibit the condition or disease. In another embodiment, the control of reference levels comprises that of a housekeeping marker whose level is minimally affected, if at all, in different biological settings such as diseased versus non-diseased states. In yet another embodiment, the control or reference level comprises that of the level of the same marker in the same subject but in a sample taken at a different time point. Other types of controls are described herein.

Nucleic acid biomarkers include various RNA or DNA species. For example, the biomarker can be mRNA, microRNA (miRNA), small nucleolar RNAs (snoRNA), small nuclear RNAs (snRNA), ribosomal RNAs (rRNA), heterogeneous nuclear RNA (hnRNA), ribosomal RNAS (rRNA), siRNA, transfer RNAs (tRNA), or shRNA. The DNA can be double-stranded DNA, single stranded DNA, complementary DNA, or noncoding DNA. miRNAs are short ribonucleic acid (RNA) molecules which average about 22 nucleotides long. miRNAs act as post-transcriptional regulators that bind to complementary sequences in the three prime untranslated regions (3′ UTRs) of target messenger RNA transcripts (mRNAs), which can result in gene silencing. One miRNA may act upon 1000s of mRNAs. miRNAs play multiple roles in negative regulation, e.g., transcript degradation and sequestering, translational suppression, and may also have a role in positive regulation, e.g., transcriptional and translational activation. By affecting gene regulation, miRNAs can influence many biologic processes. Different sets of expressed miRNAs are found in different cell types and tissues.

Biomarkers for use with the invention further include peptides, polypeptides, or proteins, which terms are used interchangeably throughout unless otherwise noted. In some embodiments, the protein biomarker comprises its modification state, truncations, mutations, expression level (such as overexpression or underexpression as compared to a reference level), and/or post-translational modifications, such as described above. In a non-limiting example, a biosignature for a disease can include a protein having a certain post-translational modification that is more prevalent in a sample associated with the disease than without.

A biosignature may include a number of the same type of biomarkers (e.g., two or more different microRNA or mRNA species) or one or more of different types of biomarkers (e.g. mRNAs, miRNAs, proteins, peptides, ligands, and antigens).

One or more biosignatures can comprise at least one biomarker selected from those listed in FIGS. 1, 3-60 of International Patent Application Serial No. PCT/US2011/031479, entitled “Circulating Biomarkers for Disease” and filed Apr. 6, 2011, which application is incorporated by reference in its entirety herein. A specific cell-of-origin biosignature may include one or more biomarkers. FIGS. 3-58 of International Patent Application Serial No. PCT/US2011/031479 depict tables which lists a number of disease or condition specific biomarkers that can be derived and analyzed from a vesicle. The biomarker can also be CD24, midkine, hepcidin, TMPRSS2-ERG, PCA-3, PSA, EGFR, EGFRvIII, BRAF variant, MET, cKit, PDGFR, Wnt, beta-catenin, K-ras, H-ras, N-ras, Raf, N-myc, c-myc, IGFR, PI3K, Akt, BRCA1, BRCA2, PTEN, VEGFR-2, VEGFR-1, Tie-2, TEM-1, CD276, HER-2, HER-3, or HER-4. The biomarker can also be annexin V, CD63, Rab-5b, or caveolin, or a miRNA, such as let-7a; miR-15b; miR-16; miR-19b; miR-21; miR-26a; miR-27a; miR-92; miR-93; miR-320 or miR-20. The biomarker can also be of any gene or fragment thereof as disclosed in PCT Publication No. WO2009/100029, such as those listed in Tables 3-15 therein.

In another embodiment, a vesicle comprises a cell fragment or cellular debris derived from a rare cell, such as described in PCT Publication No. WO2006054991. One or more biomarkers, such as CD 146, CD 105, CD31, CD 133, CD 106, or a combination thereof, can be assessed for the vesicle. In one embodiment, a capture agent for the one or more biomarkers is used to isolate or detect a vesicle. In some embodiments, one or more of the biomarkers CD45, cytokeratin (CK) 8, CK18, CK19, CK20, CEA, EGFR, GUC, EpCAM, VEGF, TS, Muc-1, or a combination thereof is assessed for a vesicle. In one embodiment, a tumor-derived vesicle is CD45−, CK+ and comprises a nucleic acid, wherein the membrane vesicle has an absence of, or low expression or detection of CD45, has detectable expression of a cytokeratin (such as CK8, CK18, CK19, or CK20), and detectable expression of a nucleic acid.

Any number of useful biomarkers that can be assessed as part of a vesicle biosignature are disclosed throughout the application, including without limitation CD9, EphA2, EGFR, B7H3, PSM, PCSA, CD63, STEAP, CD81, ICAM1, A33, DR3, CD66e, MFG-E8, TROP-2, Mammaglobin, Hepsin, NPGP/NPFF2, PSCA, 5T4, NGAL, EpCam, neurokinin receptor-1 (NK-1 or NK-1R), NK-2, Pai-1, CD45, CD10, HER2/ERBB2, AGTR1, NPY1R, MUC1, ESA, CD133, GPR30, BCA225, CD24, CA15.3 (MUC1 secreted), CA27.29 (MUC1 secreted), NMDAR1, NMDAR2, MAGEA, CTAG1B, NY-ESO-1, SPB, SPC, NSE, PGP9.5, P2RX7, NDUFB7, NSE, GAL3, osteopontin, CHI3L1, IC3b, mesothelin, SPA, AQPS, GPCR, hCEA-CAM, PTP IA-2, CABYR, TMEM211, ADAM28, UNC93A, MUC17, MUC2, IL10R-beta, BCMA, HVEM/TNFRSF14, Trappin-2 Elafin, ST2/IL1 R4, TNFRF14, CEACAM1, TPA1, LAMP, WF, WH1000, PECAM, BSA, TNFR, or a combination thereof.

Other biomarkers useful for assessment in methods and compositions disclosed herein include those associated with conditions or physiological states as disclosed in U.S. Pat. Nos. 6,329,179 and 7,625,573; U.S. Patent Publication Nos. 2002/106684, 2004/005596, 2005/0159378, 2005/0064470, 2006/116321, 2007/0161004, 2007/0077553, 2007/104738, 2007/0298118, 2007/0172900, 2008/0268429, 2010/0062450, 2007/0298118, 2009/0220944 and 2010/0196426; U.S. patent application Ser. Nos. 12/524,432, 12/524,398, 12/524,462; Canadian Patent CA 2453198; and International PCT Patent Publication Nos. WO1994022018, WO2001036601, WO2003063690, WO2003044166, WO2003076603, WO2005121369, WO2005118806, WO/2005/078124, WO2007126386, WO2007088537, WO2007103572, WO2009019215, WO2009021322, WO2009036236, WO2009100029, WO2009015357, WO2009155505, WO 2010/065968 and WO 2010/070276; each of which patent or application is incorporated herein by reference in their entirety. The biomarkers disclosed in these patents and applications, including vesicle biomarkers and microRNAs, can be assessed as part of a signature for characterizing a phenotype, such as providing a diagnosis, prognosis or theranosis of a cancer or other disease. Furthermore, the methods and techniques disclosed therein can be used to assess biomarkers, including vesicle biomarkers and microRNAs.

Another group of useful biomarkers for assessment in methods and compositions disclosed herein include those associated with cancer diagnostics, prognostics and theranostics as disclosed in U.S. Pat. Nos. 6,692,916, 6,960,439, 6,964,850, 7,074,586; U.S. patent application Ser. Nos. 11/159,376, 11/804,175, 12/594,128, 12/514,686, 12/514,775, 12/594,675, 12/594,911, 12/594,679, 12/741,787, 12/312,390; and International PCT Patent Application Nos. PCT/US2009/049935, PCT/US2009/063138, PCT/US2010/000037; each of which patent or application is incorporated herein by reference in their entirety. Useful biomarkers further include those described in U.S. patent application Ser. No. 10/703,143 and U.S. Ser. No. 10/701,391 for inflammatory disease; Ser. No. 11/529,010 for rheumatoid arthritis; Ser. No. 11/454,553 and Ser. No. 11/827,892 for multiple sclerosis; Ser. No. 11/897,160 for transplant rejection; Ser. No. 12/524,677 for lupus; PCT/US2009/048684 for osteoarthritis; Ser. No. 10/742,458 for infectious disease and sepsis; Ser. No. 12/520,675 for sepsis; each of which patent or application is incorporated herein by reference in their entirety. The biomarkers disclosed in these patents and applications, including mRNAs, can be assessed as part of a signature for characterizing a phenotype, such as providing a diagnosis, prognosis or theranosis of a cancer or other disease. Furthermore, the methods and techniques disclosed therein can be used to assess biomarkers, including vesicle biomarkers and microRNAs.

Still other biomarkers useful for assessment in methods and compositions disclosed herein include those associated with conditions or physiological states as disclosed in Wieczorek et al., Isolation and characterization of an RNA-proteolipid complex associated with the malignant state in humans, Proc Natl Acad Sci USA. 1985 May; 82(10):3455-9; Wieczorek et al., Diagnostic and prognostic value of RNA-proteolipid in sera of patients with malignant disorders following therapy: first clinical evaluation of a novel tumor marker, Cancer Res. 1987 Dec. 1; 47(23):6407-12; Escola et al. Selective enrichment of tetraspan proteins on the internal vesicles of multivesicular endosomes and on exosomes secreted by human B-lymphocytes. J. Biol. Chem. (1998) 273:20121-27; Pileri et al. Binding of hepatitis C virus to CD81 Science, (1998) 282:938-41); Kopreski et al. Detection of Tumor Messenger RNA in the Serum of Patients with Malignant Melanoma, Clin. Cancer Res. (1999) 5:1961-1965; Carr et al. Circulating Membrane Vesicles in Leukemic Blood, Cancer Research, (1985) 45:5944-51; Weichert et al. Cytoplasmic CD24 expression in colorectal cancer independently correlates with shortened patient survival. Clinical Cancer Research, 2005, 11:6574-81); Iorio et al. MicroRNA gene expression deregulation in human breast cancer. Cancer Res (2005) 65:7065-70; Taylor et al. Tumour-derived exosomes and their role in cancer-associated T-cell signaling defects British J Cancer (2005) 92:305-11; Valadi et al. Exosome-mediated transfer of mRNAs and microRNAs is a novel mechanism of genetic exchange between cells Nature Cell Biol (2007) 9:654-59; Taylor et al. Pregnancy-associated exosomes and their modulation of T cell signaling J Immunol (2006) 176:1534-42; Koga et al. Purification, characterization and biological significance of tumor-derived exosomes Anticancer Res(2005) 25:3703-08; Seligson et al. Epithelial cell adhesion molecule (KSA) expression: pathobiology and its role as an independent predictor of survival in renal cell carcinoma Clin Cancer Res (2004) 10:2659-69; Clayton et al. (Antigen presentingcell exosomes are protected from complement-mediated lysis by expression of CD55 and CD59. Eur J Immunol (2003) 33:522-31); Simak et al. Cell Membrane Microparticles in Blood and Blood Products: Potentially Pathogenic Agents and Diagnostic Markers Trans Med Reviews (2006) 20:1-26; Choi et al. Proteomic analysis of microvesicles derived from human colorectal cancer cells J Proteome Res (2007) 6:4646-4655; Iero et al. Tumour-released exosomes and their implications in cancer immunity Cell Death Diff (2008) 15:80-88; Baj-Krzyworzeka et al. Tumour-derived microvesicles carry several surface determinants and mRNA of tumour cells and transfer some of these determinants to monocytes Cancer Immunol Immunother (2006) 55:808-18; Admyre et al. B cell-derived exosomes can present allergen peptides and activate allergen-specific T cells to proliferate and produce TH2-like cytokines J Allergy Clin Immunol (2007) 120:1418-1424; Aoki et al. Identification and characterization of microvesicles secreted by 3T3-L1 adipocytes: redox- and hormone dependent induction of milk fat globule-epidermal growth factor 8-associated microvesicles Endocrinol (2007) 148:3850-3862; Baj-Krzyworzeka et al. Tumour-derived microvesicles carry several surface determinants and mRNA of tumour cells and transfer some of these determinants to monocytes Cencer Immunol Immunother (2006) 55:808-18; Skog et al. Glioblastoma microvesicles transport RNA and proteins that promote tumour growth and provide diagnostic biomarkers Nature Cell Biol (2008) 10:1470-76; El-Hefnawy et al. Characterization of amplifiable, circulating RNA in plasma and its potential as a tool for cancer diagnostics Clin Chem (2004) 50:564-573; Pisitkun et al., Proc Natl Acad Sci USA, 2004; 101:13368-13373; Mitchell et al., Can urinary exosomes act as treatment response markers in Prostate Cancer?, Journal of Translational Medicine 2009, 7:4; Clayton et al., Human Tumor-Derived Exosomes Selectively Impair Lymphocyte Responses to Interleukin-2, Cancer Res 2007; 67: (15). Aug. 1, 2007; Rabesandratana et al. Decay-accelerating factor (CD55) and membrane inhibitor of reactive lysis (CD59) are released within exosomes during In vitro maturation of reticulocytes. Blood 91:2573-2580 (1998); Lamparski et al. Production and characterization of clinical grade exosomes derived from dendritic cells. J Immunol Methods 270:211-226 (2002); Keller et al. CD24 is a marker of exosomes secreted into urine and amniotic fluid. Kidney Int'l 72:1095-1102 (2007); Runz et al. Malignant ascites-derived exosomes of ovarian carcinoma patients contain CD24 and EpCAM. Gyn Oncol 107:563-571 (2007); Redman et al. Circulating microparticles in normal pregnancy and preeclampsia placenta. 29:73-77 (2008); Gutwein et al. Cleavage of L 1 in exosomes and apoptotic membrane vesicles released from ovarian carcinoma cells. Clin Cancer Res 11:2492-2501 (2005); Kristiansen et al., CD24 is an independent prognostic marker of survival in nonsmall cell lung cancer patients, Brit J Cancer 88:231-236 (2003); Lim and Oh, The Role of CD24 in Various Human Epithelial Neoplasias, Pathol Res Pract 201:479-86 (2005); Matutes et al., The Immunophenotype of Splenic Lymphoma with Villous Lymphocytes and its Relevance to the Differential Diagnosis With Other B-Cell Disorders, Blood 83:1558-1562 (1994); Pirruccello and Lang, Differential Expression of CD24-Related Epitopes in Mycosis Fungoides/Sezary Syndrome: A Potential Marker for Circulating Sezary Cells, Blood 76:2343-2347 (1990). The biomarkers disclosed in these publications, including vesicle biomarkers and microRNAs, can be assessed as part of a signature for characterizing a phenotype, such as providing a diagnosis, prognosis or theranosis of a cancer or other disease. Furthermore, the methods and techniques disclosed therein can be used to assess biomarkers, including vesicle biomarkers and microRNAs.

Still other biomarkers useful for assessment in methods and compositions disclosed herein include those associated with conditions or physiological states as disclosed in Rajendran et al., Proc Natl Acad Sci US A 2006; 103:11172-11177, Taylor et al., Gynecol Oncol 2008; 110:13-21, Zhou et al., Kidney Int 2008; 74:613-621, Buning et al., Immunology 2008, Prado et al. J Immunol 2008; 181:1519-1525, Vella et al. (2008) Vet Immunol Immunopathol 124(3-4): 385-93, Gould et al. (2003). Proc Natl Acad Sci US A 100(19): 10592-7, Fang et al. (2007). PLoS Biol 5(6): e158, Chen, B. J. and R. A. Lamb (2008). Virology 372(2): 221-32, Bhatnagar, S. and J. S. Schorey (2007). J Biol Chem 282(35): 25779-89, Bhatnagar et al. (2007) Blood 110(9): 3234-44, Yuyama, et al. (2008). J Neurochem 105(1): 217-24, Gomes et al. (2007). Neurosci Lett 428(1): 43-6, Nagahama et al. (2003). Autoimmunity 36(3): 125-31, Taylor, D. D., S. Akyol, et al. (2006). J Immunol 176(3): 1534-42, Peche, et al. (2006). Am J Transplant 6(7): 1541-50, Zero, M., M. Valenti, et al. (2008). Cell Death and Differentiation 15: 80-88, Gesierich, S., I. Berezoversuskiy, et al. (2006), Cancer Res 66(14): 7083-94, Clayton, A., A. Turkes, et al. (2004). Faseb J 18(9): 977-9, Skriner., K Adolph, et al. (2006). Arthritis Rheum 54(12): 3809-14, Brouwer, R., G. J. Pruijn, et al. (2001). Arthritis Res 3(2): 102-6, Kim, S. H., N Bianco, et al. (2006). Mol Ther 13(2): 289-300, Evans, C. H., S. C. Ghivizzani, et al. (2000). Clin Orthop Relat Res (379 Suppl): S300-7, Zhang, H. G., C. Liu, et al. (2006). J Immunol 176(12): 7385-93, Van Niel, G., J. Mallegol, et al. (2004). Gut 52: 1690-1697, Fiasse, R. and O. Dewit (2007). Expert Opinion on Therapeutic Patents 17(12): 1423-1441(19). The biomarkers disclosed in these publications, including vesicle biomarkers and microRNAs, can be assessed as part of a signature for characterizing a phenotype, such as providing a diagnosis, prognosis or theranosis of a cancer or other disease. Furthermore, the methods and techniques disclosed therein can be used to assess biomarkers, including vesicle biomarkers and microRNAs.

In another aspect, the invention provides a method of assessing a cancer comprising detecting a level of one or more circulating biomarkers in a sample from a subject selected from the group consisting of CD9, HSP70, Gal3, MIS, EGFR, ER, ICB3, CD63, B7H4, MUC1, DLL4, CD81, ERB3, VEGF, BCA225, BRCA, CA125, CD174, CD24, ERB2, NGAL, GPR30, CYFRA21, CD31, cMET, MUC2 or ERB4. CD9, HSP70, Gal3, MIS, EGFR, ER, ICB3, CD63, B7H4, MUC1, DLL4, CD81, ERB3, VEGF, BCA225, BRCA, BCA200, CA125, CD174, CD24, ERB2, NGAL, GPR30, CYFRA21, CD31, cMET, MUC2 or ERB4. In another embodiment, the one or more circulating biomarkers are selected from the group consisting of CD9, EphA2, EGFR, B7H3, PSMA, PCSA, CD63, STEAP, STEAP, CD81, B7H3, STEAP1, ICAM1 (CD54), PSMA, A33, DR3, CD66e, MFG-8e, EphA2, Hepsin, TMEM211, EphA2, TROP-2, EGFR, Mammoglobin, Hepsin, NPGP/NPFF2, PSCA, 5T4, NGAL, NK-2, EpCam, NGAL, NK-1R, PSMA, 5T4, PAI-1, and CD45. In still another embodiment, the one or more circulating biomarkers are selected from the group consisting of CD9, MIS Rii, ER, CD63, MUC1, HER3, STAT3, VEGFA, BCA, CA125, CD24, EPCAM, and ERB B4. Any number of useful biomarkers can be assessed from these groups, e.g., 1, 2, 3, 4, 5, 6, 7, 8, 9, 10 or more. In some embodiments, the one or more biomarkers are one or more of Gal3, BCA200, OPN and NCAM, e.g., Gal3 and BCA200, OPN and NCAM, or all four. Assessing the cancer may comprise diagnosing, prognosing or theranosing the cancer. The cancer can be a breast cancer. The markers can be associated with a vesicle or vesicle population. For example, the one or more circulating biomarker can be a vesicle surface antigen or vesicle payload. Vesicle surface antigens can further be used as capture antigens, detector antigens, or both.

The invention further provides a method for predicting a response to a therapeutic agent comprising detecting a level of one or more circulating biomarkers in a sample from a subject selected from the group consisting of CD9, HSP70, Gal3, MIS, EGFR, ER, ICB3, CD63, B7H4, MUC1, DLL4, CD81, ERB3, VEGF, BCA225, BRCA, CA125, CD174, CD24, ERB2, NGAL, GPR30, CYFRA21, CD31, cMET, MUC2 or ERB4. Biomarkers can also be selected from the group consisting of CD9, EphA2, EGFR, B7H3, PSMA, PCSA, CD63, STEAP, STEAP, CD81, B7H3, STEAP1, ICAM1 (CD54), PSMA, A33, DR3, CD66e, MFG-8e, EphA2, Hepsin, TMEM211, EphA2, TROP-2, EGFR, Mammoglobin, Hepsin, NPGP/NPFF2, PSCA, 5T4, NGAL, NK-2, EpCam, NGAL, NK-1R, PSMA, 5T4, PAI-1, and CD45. In still another embodiment, the one or more circulating biomarkers are selected from the group consisting of CD9, MIS Rii, ER, CD63, MUC1, HER3, STAT3, VEGFA, BCA, CA125, CD24, EPCAM, and ERB B4. Any number of useful biomarkers can be assessed from these groups, e.g., 1, 2, 3, 4, 5, 6, 7, 8, 9, 10 or more. In some embodiments, the one or more biomarkers are one or more of Gal3, BCA200, OPN and NCAM, e.g., Gal3 and BCA200, OPN and NCAM, or all four. The therapeutic agent can be a therapeutic agent for treating cancer. The cancer can be a breast cancer. The markers can be associated with a vesicle or vesicle population. For example, the one or more circulating biomarker can be a vesicle surface antigen or vesicle payload. Vesicle surface antigens can further be used as capture antigens, detector antigens, or both.

Various methods or platforms can be used to assess or detect biomarkers identified herein. Examples of such methods or platforms include but are not limited to using an antibody array, microbeads, or other method disclosed herein or known in the art. For example, a capture antibody or aptamer to the one or more biomarkers can be bound to the array or bead. The captured vesicles can then be detected using a detectable agent. In some embodiments, captured vesicles are detected using an agent, e.g., an antibody or aptamer, that recognizes general vesicle biomarkers that detect the overall population of vesicles, such as a tetraspanin or MFG-E8. These can include tetraspanins such as CD9, CD63 and/or CD81. In other embodiments, the captured vesicles are detected using markers specific for vesicle origin, e.g., a type of tissue or organ. In some embodiments, the captured vesicles are detected using CD31, a marker for cells or vesicles of endothelial origin. As desired, the biomarkers used for capture can also be used for detection, and vice versa.

Methods of the invention can be used to assess various diseases or conditions, where biomarkers correspond to various such diseases or conditions. For example, methods of the invention are applied to assess one or more cancers, such as those disclosed herein, wherein a method comprises detecting a level of one or more circulating biomarker in a sample from a subject selected from the group consisting of 5T4 (trophoblast), ADAM10, AGER/RAGE, APC, APP (β-amyloid), ASPH (A-10), B7H3 (CD276), BACE1, BAI3, BRCA1, BDNF, BIRC2, C1GALT1, CA125 (MUC16), Calmodulin 1, CCL2 (MCP-1), CD9, CD10, CD127 (IL7R), CD174, CD24, CD44, CD63, CD81, CEA, CRMP-2, CXCR3, CXCR4, CXCR6, CYFRA 21, derlin 1, DLL4, DPP6, E-CAD, EpCaM, EphA2 (H-77), ER(1) ESR1 α, ER(2) ESR2 β, Erb B4, Erbb2, erb3 (Erb-B3), PA2G4, FRT (FLT1), Gal3, GPR30 (G-coupled ER1), HAP1, HER3, HSP-27, HSP70, IC3b, IL8, insig, junction plakoglobin, Keratin 15, KRAS, Mammaglobin, MART1, MCT2, MFGE8, MMP9, MRP8, Muc1, MUC17, MUC2, NCAM, NG2 (CSPG4), Ngal, NHE-3, NT5E (CD73), ODC1, OPG, OPN, p53, PARK7, PCSA, PGP9.5 (PARKS), PR(B), PSA, PSMA, RAGE, STXBP4, Survivin, TFF3 (secreted), TIMP1, TIMP2, TMEM211, TRAF4 (scaffolding), TRAIL-R2 (death Receptor 5), TrkB, Tsg 101, UNC93a, VEGF A, VEGFR2, YB-1, VEGFR1, GCDPF-15 (PIP), BigH3 (TGFbl-induced protein), 5HT2B (serotonin receptor 2B), BRCA2, BACE 1, CDH1-cadherin. The methods can comprise detecting protein, RNA or DNA of the specified target biomarker. The one or more marker can be assessed directly from a biological fluid, such as those fluids disclosed herein, or can be assessed for its association with a vesicle, e.g., as a vesicle surface antigen or as vesicle payload (e.g., soluble protein, mRNA or DNA). A particular biosignature determined using methods and compositions of the invention can comprise any number of useful biomarkers, e.g., a biosignature can comprise 1, 2, 3, 4, 5, 6, 7, 8, 9, 10 or more different biomarkers (or in some cases different molecules of the same biomarkers, such protein and nucleic acid). Vesicle surface antigens can also be used as capture antigens, detector antigens, or both, as disclosed herein or in applications incorporated by reference.

Methods and compositions of the invention are applied to assess various aspects of a cancer, including identifying different informative aspects of a cancer, e.g., identifying a biosignature that is indicative of metastasis, angiogenesis, or classifying different stages, classes or subclasses of the same tumor or tumor lineage.

Furthermore, methods of the invention comprise determining if a disease or condition affects immunomodulation in a subject. For example, the one or more circulating biomarker for immunomodulation can be one or more of CD45, FasL, CTLA4, CD80 and CD83. The one or more circulating biomarker for metastatis can be one or more of Muc1, CD147, TIMP1, TIMP2, MMP7, and MMP9. The one or more circulating biomarker for angiogenesis can be one or more of HIF2a, Tie2, Ang1, DLL4 and VEGFR2. Any number of useful biomarkers can be assessed from the groups, e.g., 1, 2, 3, 4, 5, 6, 7, 8, 9, 10 or more. The cancer can be a breast cancer. The markers can be associated with a vesicle or vesicle population. For example, the one or more circulating biomarker can be a vesicle surface antigen or vesicle payload. Vesicle surface antigens can further be used as capture antigens, detector antigens, or both.

A biosignature can comprise DLL4 or cMET. Delta-like 4 (DLL4) is a Notch-ligand and is upregulated during angiogenesis. cMET (also referred to as c-Met, MET, or MNNG HOS Transforming gene) is a proto-oncogene that encodes a membrane receptor tyrosine kinase whose ligand is hepatocyte growth factor (HGF). The MET protein is sometimes referred to as the hepatocyte growth factor receptor (HGFR). MET is normally expressed on epithelial cells, and improper activation can trigger tumor growth, angiogenesis and metastasis. DLL4 and cMET can be used as biomarkers to detect a vesicle population.

Biomarkers that can be derived and analyzed from a vesicle include miRNA (miR), miRNA*nonsense (miR*), and other RNAs (including, but not limited to, mRNA, preRNA, priRNA, hnRNA, snRNA, siRNA, shRNA). A miRNA biomarker can include not only its miRNA and microRNA* nonsense, but its precursor molecules: pri-microRNAs (pri-miRs) and pre-microRNAs (pre-miRs). The sequence of a miRNA can be obtained from publicly available databases such as http://www.mirbase.org/, http://www.microrna.org/, or any others available. Unless noted, the terms miR, miRNA and microRNA are used interchangeably throughout unless noted. In some embodiments, the methods of the invention comprise isolating vesicles, and assessing the miRNA payload within the isolated vesicles. The biomarker can also be a nucleic acid molecule (e.g. DNA), protein, or peptide. The presence or absence, expression level, mutations (for example genetic mutations, such as deletions, translocations, duplications, nucleotide or amino acid substitutions, and the like) can be determined for the biomarker. Any epigenetic modulation or copy number variation of a biomarker can also be analyzed.

The one or more biomarkers analyzed can be indicative of a particular tissue or cell of origin, disease, or physiological state. Furthermore, the presence, absence or expression level of one or more of the biomarkers described herein can be correlated to a phenotype of a subject, including a disease, condition, prognosis or drug efficacy. The specific biomarker and biosignature set forth below constitute non-inclusive examples for each of the diseases, condition comparisons, conditions, and/or physiological states. Furthermore, the one or more biomarker assessed for a phenotype can be a cell-of-origin specific vesicle.

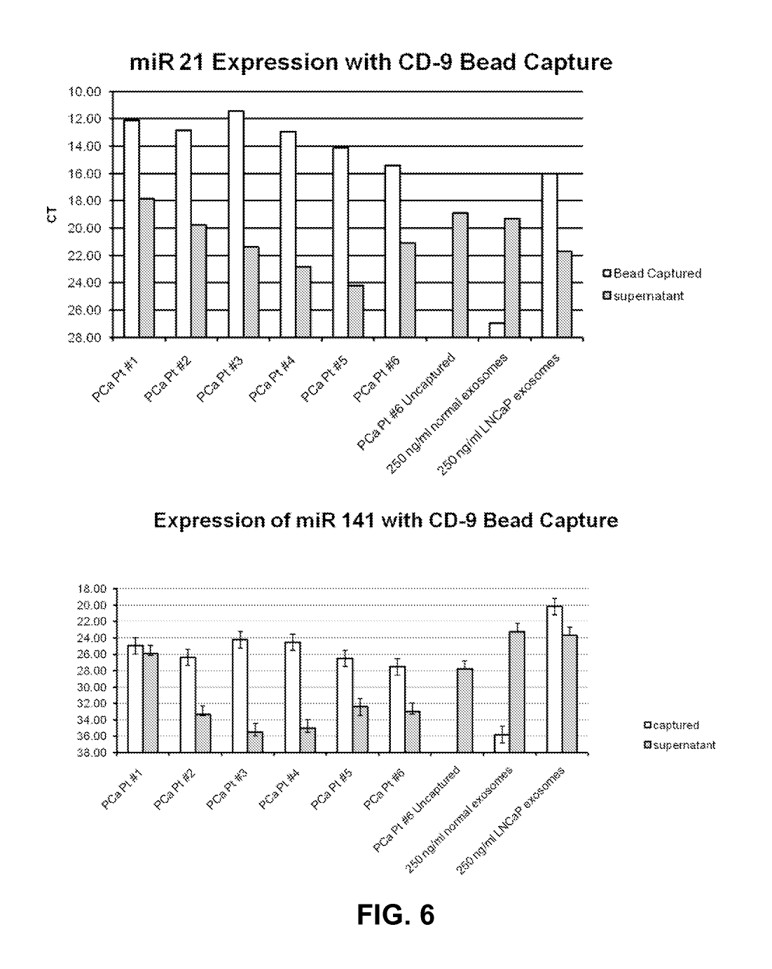

The one or more miRNAs used to characterize a phenotype may be selected from those disclosed in PCT Publication No. WO2009/036236. For example, one or more miRNAs listed in Tables I-VI (FIGS. 6-11) therein can be used to characterize colon adenocarcinoma, colorectal cancer, prostate cancer, lung cancer, breast cancer, b-cell lymphoma, pancreatic cancer, diffuse large BCL cancer, CLL, bladder cancer, renal cancer, hypoxia-tumor, uterine leiomyomas, ovarian cancer, hepatitis C virus-associated hepatocellular carcinoma, ALL, Alzheimer's disease, myelofibrosis, myelofibrosis, polycythemia vera, thrombocythemia, HIV, or HIV-I latency, as further described herein.

The one or more miRNAs can be detected in a vesicle. The one or more miRNAs can be miR-223, miR-484, miR-191, miR-146a, miR-016, miR-026a, miR-222, miR-024, miR-126, and miR-32. One or more miRNAs can also be detected in PBMC. The one or more miRNAs can be miR-223, miR-150, miR-146b, miR-016, miR-484, miR-146a, miR-191, miR-026a, miR-019b, or miR-020a. The one or more miRNAs can be used to characterize a particular disease or condition. For example, for the disease bladder cancer, one or more miRNAs can be detected, such as miR-223, miR-26b, miR-221, miR-103-1, miR-185, miR-23b, miR-203, miR-17-5p, miR-23a, miR-205 or any combination thereof. The one or more miRNAs may be upregulated or overexpressed.

In some embodiments, the one or more miRNAs is used to characterize hypoxia-tumor. The one or more miRNA may be miR-23, miR-24, miR-26, miR-27, miR-103, miR-107, miR-181, miR-210, or miR-213, and may be upregulated. One or more miRNAs can also be used to characterize uterine leiomyomas. For example, the one or more miRNAs used to characterize a uterine leiomyoma may be a let-7 family member, miR-21, miR-23b, miR-29b, or miR-197. The miRNA can be upregulated.

Myelofibrosis can also be characterized by one or more miRNAs, such as miR-190, which can be upregulated; miR-31, miR-150 and miR-95, which can be downregulated, or any combination thereof. Furthermore, myelofibrosis, polycythemia vera or thrombocythemia can also be characterized by detecting one or more miRNAs, such as, but not limited to, miR-34a, miR-342, miR-326, miR-105, miR-149, miR-147, or any combination thereof. The one or more miRNAs may be downregulated.

Other examples of phenotypes that can be characterized by assessing a vesicle for one or more biomarkers are father described herein.

The one or more biomarkers can be detected using a probe. A probe can comprise an oligonucleotide, such as DNA or RNA, an aptamer, monoclonal antibody, polyclonal antibody, Fabs, Fab′, single chain antibody, synthetic antibody, peptoid, zDNA, peptide nucleic acid (PNA), locked nucleic acid (LNA), lectin, synthetic or naturally occurring chemical compound (including but not limited to a drug or labeling reagent), dendrimer, or a combination thereof. The probe can be directly detected, for example by being directly labeled, or be indirectly detected, such as through a labeling reagent. The probe can selectively recognize a biomarker. For example, a probe that is an oligonucleotide can selectively hybridize to a miRNA biomarker.

In aspects, the invention provides for the diagnosis, theranosis, prognosis, disease stratification, disease staging, treatment monitoring or predicting responder/non-responder status of a disease or disorder in a subject. The invention comprises assessing vesicles from a subject, including assessing biomarkers present on the vesicles and/or assessing payload within the vesicles, such as protein, nucleic acid or other biological molecules. Any appropriate biomarker that can be assessed using a vesicle and that relates to a disease or disorder can be used the carry out the methods of the invention. Furthermore, any appropriate technique to assess a vesicle as described herein can be used. Exemplary biomarkers for specific diseases that can be assessed according to the methods of the invention include the biomarkers described in International Patent Application Serial No. PCT/US2011/031479, entitled “Circulating Biomarkers for Disease” and filed Apr. 6, 2011, which application is incorporated by reference in its entirety herein.

Any of the types of biomarkers or specific biomarkers described herein can be assessed to identify a biosignature or to identify a candidate biosignature. Exemplary biomarkers include without limitation those in Table 3, Table 4 or Table 5. The markers in the table can be used for capture and/or detection of vesicles for characterizing phenotypes as disclosed herein. In some cases, multiple capture and/or detectors are used to enhance the characterization. The markers can be detected as protein or as mRNA, which can be circulating freely or in a complex with other biological molecules. The markers can be detected as vesicle surface antigens or and vesicle payload. The “Illustrative Class” indicates indications for which the markers are known markers. Those of skill will appreciate that the markers can also be used in alternate settings in certain instances. For example, a marker which can be used to characterize one type disease may also be used to characterize another disease as appropriate. Consider a non-limiting example of a tumor marker which can be used as a biomarker for tumors from various lineages. The biomarker references in Table 5 are those commonly used in the art. Gene aliases and descriptions can be found using a variety of online databases, including GeneCards® (www.genecards.org), HUGO Gene Nomenclature (www.genenames.org), Entrez Gene (www.ncbi.nlm.nih.gov/entrez/query.fcgidbgene), UniProtKB/Swiss-Prot (www.uniprot.org), UniProtKB/TrEMBL (www.uniprot.org), OMIM (www.ncbi.nlm.nih.gov/entrez/query.fcgidbOMIM), GeneLoc (genecards.weizmann.ac.il/geneloc/), and Ensembl (www.ensembl.org). Generally, gene symbols and names below correspond to those approved by HUGO, and protein names are those recommended by UniProtKB/Swiss-Prot. Common alternatives are provided as well. In some cases, biomarkers are referred to by Ensembl reference numbers, which are of the form “ENSG” followed by a number, e.g., ENSG00000005893 which corresponds to LAMP2. In Table 5, solely for sake of brevity, “E.” is sometimes used to represent “ENSG00000”. For example, “E.005893 represents “ENSG00000005893.” Where a protein name indicates a precursor, the mature protein is also implied. Throughout the application, gene and protein symbols may be used interchangeably and the meaning can be derived from context as necessary.

TABLE 5
Illustrative Biomarkers

| Illustrative Class | Biomarkers |
|---|---|
| Drug associated | ABCC1, ABCG2, ACE2, ADA, ADH1C, ADH4, AGT, AR, AREG, ASNS, BCL2, BCRP, |
| targets and | BDCA1, beta III tubulin, BIRC5, B-RAF, BRCA1, BRCA2, CA2, caveolin, CD20, CD25, |
| prognostic markers | CD33, CD52, CDA, CDKN2A, CDKN1A, CDKN1B, CDK2, CDW52, CES2, CK 14, CK |
| | 17, CK 5/6, c-KIT, c-Met, c-Myc, COX-2, Cyclin D1, DCK, DHFR, DNMT1, DNMT3A, |
| | DNMT3B, E-Cadherin, ECGF1, EGFR, EML4-ALK fusion, EPHA2, Epiregulin, ER, |
| | ERBR2, ERCC1, ERCC3, EREG, ESR1, FLT1, folate receptor, FOLR1, FOLR2, FSHB, |
| | FSHPRH1, FSHR, FYN, GART, GNA11, GNAQ, GNRH1, GNRHR1, GSTP1, HCK, |
| | HDAC1, hENT-1, Her2/Neu, HGF, HIF1A, HIG1, HSP90, HSP90AA1, HSPCA, IGF-1R, |
| | IGFRBP, IGFRBP3, IGFRBP4, IGFRBP5, IL13RA1, IL2RA, KDR, Ki67, KIT, K-RAS, |
| | LCK, LTB, Lymphotoxin Beta Receptor, LYN, MET, MGMT, MLH1, MMR, MRP1, |
| | MS4A1, MSH2, MSH5, Myc, NFKB1, NFKB2, NFKBIA, NRAS, ODC1, OGFR, p16, p21, |
| | p27, p53, p95, PARP-1, PDGFC, PDGFR, PDGFRA, PDGFRB, PGP, PGR, PI3K, POLA, |
| | POLA1, PPARG, PPARGC1, PR, PTEN, PTGS2, PTPN12, RAF1, RARA, ROS1, RRM1, |
| | RRM2, RRM2B, RXRB, RXRG, SIK2, SPARC, SRC, SSTR1, SSTR2, SSTR3, SSTR4, |
| | SSTR5, Survivin, TK1, TLE3, TNF, TOP1, TOP2A, TOP2B, TS, TUBB3, TXN, TXNRD1, |
| | TYMS, VDR, VEGF, VEGFA, VEGFC, VHL, YES1, ZAP70 |
| Drug associated | ABL1, STK11, FGFR2, ERBB4, SMARCB1, CDKN2A, CTNNB1, FGFR1, FLT3, |
| targets and | NOTCH1, NPM1, SRC, SMAD4, FBXW7, PTEN, TP53, AKT1, ALK, APC, CDH1, C-Met, |
| prognostic markers | HRAS, IDH1, JAK2, MPL, PDGFRA, SMO, VHL, ATM, CSF1R, FGFR3, GNAS, ERBB2, |
| | HNF1A, JAK3, KDR, MLH1, PTPN11, RB1, RET, c-Kit, EGFR, PIK3CA, NRAS, GNA11, |
| | GNAQ, KRAS, BRAF |
| Drug associated | ALK, AR, BRAF, cKIT, cMET, EGFR, ER, ERCC1, GNA11, HER2, IDH1, KRAS, MGMT, |
| targets and | MGMT promoter methylation, NRAS, PDGFRA, Pgp, PIK3CA, PR, PTEN, ROS1, RRM1, |
| prognostic markers | SPARC, TLE3, TOP2A, TOPO1, TS, TUBB3, VHL |
| Drug associated | AR, cMET, EGFR, ER, HER2, MGMT, Pgp, PR, PTEN, RRM1, SPARC, TLE3, TOPO1, |
| targets and | TOP2A, TS, TUBB3, ALK, cMET, HER2, ROS1, TOP2A, BRAF, IDH2, MGMT |
| prognostic markers | Methylation, ABL1, AKT1, ALK, APC, ATM, BRAF, CDH1, CSF1R, CTNNB1, EGFR, |
| | ERBB2 (HER2), ERBB4, FBXW7, FGFR1, FGFR2, FLT3, GNA11, GNAQ, GNAS, |
| | HNF1A, HRAS, IDH1, JAK2, JAK3, KDR (VEGFR2), KIT, KRAS, MET, MLH1, MPL, |
| | NOTCH1, NPM1, NRAS, PDGFRA, PIK3CA, PTEN, PTPN11, RB1, RET, SMAD4, |
| | SMARCB1, SMO, STK11, TP53, VHL |
| 5-aminosalicyclic | μ-protocadherin, KLF4, CEBPα |
| acid (5-ASA) | |
| efficacy | |
| Cancer treatment | AR, AREG (Amphiregulin), BRAF, BRCA1, cKIT, cMET, EGFR, EGFR w/T790M, EML4- |
| associated markers | ALK, ER, ERBB3, ERBB4, ERCC1, EREG, GNA11, GNAQ, hENT-1, Her2, Her2 Exon 20 |
| | insert, IGF1R, Ki67, KRAS, MGMT, MGMT methylation, MSH2, MSI, NRAS, PGP |
| | (MDR1), PIK3CA, PR, PTEN, ROS1, ROS1 translocation, RRM1, SPARC, TLE3, TOPO1, |
| | TOPO2A, TS, TUBB3, VEGFR2 |
| Cancer treatment | AR, AREG, BRAF, BRCA1, cKIT, cMET, EGFR, EGFR w/T790M, EML4-ALK, ER, |
| associated markers | ERBB3, ERBB4, ERCC1, EREG, GNA11, GNAQ, Her2, Her2 Exon 20 insert, IGFR1, Ki67, |
| | KRAS, MGMT-Me, MSH2, MSI, NRAS, PGP (MDR-1), PIK3CA, PR, PTEN, ROS1 |
| | translocation, RRM1, SPARC, TLE3, TOPO1, TOPO2A, TS, TUBB3, VEGFR2 |
| Colon cancer | AREG, BRAF, EGFR, EML4-ALK, ERCC1, EREG, KRAS, MSI, NRAS, PIK3CA, PTEN, |
| treatment | TS, VEGFR2 |
| associated markers | |
| Colon cancer | AREG, BRAF, EGFR, EML4-ALK, ERCC1, EREG, KRAS, MSI, NRAS, PIK3CA, PTEN, |
| treatment | TS, VEGFR2 |
| associated markers | |
| Melanoma | BRAF, cKIT, ERBB3, ERBB4, ERCC1, GNA11, GNAQ, MGMT, MGMT methylation, |
| treatment | NRAS, PIK3CA, TUBB3, VEGFR2 |
| associated markers | |
| Melanoma | BRAF, cKIT, ERBB3, ERBB4, ERCC1, GNA11, GNAQ, MGMT-Me, NRAS, PIK3CA, |
| treatment | TUBB3, VEGFR2 |
| associated markers | |
| Ovarian cancer | BRCA1, cMET, EML4-ALK, ER, ERBB3, ERCC1, hENT-1, HER2, IGF1R, PGP(MDR1), |
| treatment | PIK3CA, PR, PTEN, RRM1, TLE3, TOPO1, TOPO2A, TS |
| associated markers | |
| Ovarian cancer | BRCA1, cMET, EML4-ALK (translocation), ER, ERBB3, ERCC1, HER2, PIK3CA, PR, |
| treatment | PTEN, RRM1, TLE3, TS |
| associated markers | |
| Breast cancer | BRAF, BRCA1, EGFR, EGFR T790M, EML4-ALK, ER, ERBB3, ERCC1, HER2, Ki67, |
| treatment | PGP (MDR1), PIK3CA, PR, PTEN, ROS1, ROS1 translocation, RRM1, TLE3, TOPO1, |
| associated markers | TOPO2A, TS |
| Breast cancer | BRAF, BRCA1, EGFR w/T790M, EML4-ALK, ER, ERBB3, ERCC1, HER2, Ki67, KRAS, |
| treatment | PIK3CA, PR, PTEN, ROS1 translocation, RRM1, TLE3, TOPO1, TOPO2A, TS |
| associated markers | |
| NSCLC cancer | BRAF, BRCA1, cMET, EGFR, EGFR w/T790M, EML4-ALK, ERCC1, Her2 Exon 20 |
| treatment | insert, KRAS, MSH2, PIK3CA, PTEN, ROS1 (trans), RRM1, TLE3, TS, VEGFR2 |
| associated markers | |
| NSCLC cancer | BRAF, cMET, EGFR, EGFR w/T790M, EML4-ALK, ERCC1, Her2 Exon 20 insert, KRAS, |
| treatment | MSH2, PIK3CA, PTEN, ROS1 translocation, RRM1, TLE3, TS |
| associated markers | |
| Mutated in cancers | AKT1, ALK, APC, ATM, BRAF, CDH1, CDKN2A, c-Kit, C-Met, CSF1R, CTNNB1, |
| | EGFR, ERBB2, ERBB4, FBXW7, FGFR1, FGFR2, FGFR3, FLT3, GNA11, GNAQ, GNAS, |
| | HNF1A, HRAS, IDH1, JAK2, JAK3, KDR, KRAS, MLH1, MPL, NOTCH1, NPM1, NRAS, |
| | PDGFRA, PIK3CA, PTEN, PTPN11, RB1, RET, SMAD4, SMARCB1, SMO, SRC, STK11, |
| | TP53, VHL |
| Mutated in cancers | ALK, BRAF, BRCA1, BRCA2, EGFR, ERRB2, GNA11, GNAQ, IDH1, IDH2, KIT, KRAS, |
| | MET, NRAS, PDGFRA, PIK3CA, PTEN, RET, SRC, TP53 |
| Mutated in cancers | AKT1, HRAS, GNAS, MEK1, MEK2, ERK1, ERK2, ERBB3, CDKN2A, PDGFRB, IFG1R, |
| | FGFR1, FGFR2, FGFR3, ERBB4, SMO, DDR2, GRB1, PTCH, SHH, PD1, UGT1A1, BIM, |
| | ESR1, MLL, AR, CDK4, SMAD4 |
| Mutated in cancers | ABL, APC, ATM, CDH1, CSFR1, CTNNB1, FBXW7, FLT3, HNF1A, JAK2, JAK3, KDR, |
| | MLH1, MPL, NOTCH1, NPM1, PTPN11, RB1, SMARCB1, STK11, VHL |
| Mutated in cancers | ABL1, AKT1, AKT2, AKT3, ALK, APC, AR, ARAF, ARFRP1, ARID1A, ARID2, ASXL1, |
| | ATM, ATR, ATRX, AURKA, AURKB, AXL, BAP1, BARD1, BCL2, BCL2L2, BCL6, |
| | BCOR, BCORL1, BLM, BRAF, BRCA1, BRCA2, BRIP1, BTK, CARD11, CBFB, CBL, |
| | CCND1, CCND2, CCND3, CCNE1, CD79A, CD79B, CDC73, CDH1, CDK12, CDK4, |
| | CDK6, CDK8, CDKN1B, CDKN2A, CDKN2B, CDKN2C, CEBPA, CHEK1, CHEK2, CIC, |
| | CREBBP, CRKL, CRLF2, CSF1R, CTCF, CTNNA1, CTNNB1, DAXX, DDR2, DNMT3A, |
| | DOT1L, EGFR, EMSY (C11orf30), EP300, EPHA3, EPHA5, EPHB1, ERBB2, ERBB3, |
| | ERBB4, ERG, ESR1, EZH2, FAM123B (WTX), FAM46C, FANCA, FANCC, FANCD2, |
| | FANCE, FANCF, FANCG, FANCL, FBXW7, FGF10, FGF14, FGF19, FGF23, FGF3, |
| | FGF4, FGF6, FGFR1, FGFR2, FGFR3, FGFR4, FLT1, FLT3, FLT4, FOXL2, GATA1, |
| | GATA2, GATA3, GID4 (C17orf39), GNA11, GNA13, GNAQ, GNAS, GPR124, GRIN2A, |
| | GSK3B, HGF, HRAS, IDH1, IDH2, IGF1R, IKBKE, IKZF1, IL7R, INHBA, IRF4, IRS2, |
| | JAK1, JAK2, JAK3, JUN, KAT6A (MYST3), KDM5A, KDM5C, KDM6A, KDR, KEAP1, |
| | KIT, KLHL6, KRAS, LRP1B, MAP2K1, MAP2K2, MAP2K4, MAP3K1, MCL1, MDM2, |
| | MDM4, MED12, MEF2B, MEN1, MET, MITF, MLH1, MLL, MLL2, MPL, MRE11A, |
| | MSH2, MSH6, MTOR, MUTYH, MYC, MYCL1, MYCN, MYD88, NF1, NF2, NFE2L2, |
| | NFKBIA, NKX2-1, NOTCH1, NOTCH2, NPM1, NRAS, NTRK1, NTRK2, NTRK3, |
| | NUP93, PAK3, PALB2, PAX5, PBRM1, PDGFRA, PDGFRB, PDK1, PIK3CA, PIK3CG, |
| | PIK3R1, PIK3R2, PPP2R1A, PRDM1, PRKAR1A, PRKDC, PTCH1, PTEN, PTPN11, |
| | RAD50, RAD51, RAF1, RARA, RB1, RET, RICTOR, RNF43, RPTOR, RUNX1, SETD2, |
| | SF3B1, SMAD2, SMAD4, SMARCA4, SMARCB1, SMO, SOCS1, SOX10, SOX2, SPEN, |
| | SPOP, SRC, STAG2, STAT4, STK11, SUFU, TET2, TGFBR2, TNFAIP3, TNFRSF14, |
| | TOP1, TP53, TSC1, TSC2, TSHR, VHL, WISP3, WT1, XPO1, ZNF217, ZNF703 |
| Gene | ALK, BCR, BCL2, BRAF, EGFR, ETV1, ETV4, ETV5, ETV6, EWSR1, MLL, MYC, |
| rearrangement in | NTRK1, PDGFRA, RAF1, RARA, RET, ROS1, TMPRSS2 |
| cancer | |
| Cancer Related | ABL1, ACE2, ADA, ADH1C, ADH4, AGT, AKT1, AKT2, AKT3, ALK, APC, AR, ARAF, |
| | AREG, ARFRP1, ARID1A, ARID2, ASNS, ASXL1, ATM, ATR, ATRX, AURKA, |
| | AURKB, AXL, BAP1, BARD1, BCL2, BCL2L2, BCL6, BCOR, BCORL1, BCR, BIRC5 |
| | (survivin), BLM, BRAF, BRCA1, BRCA2, BRIP1, BTK, CA2, CARD11, CAV, CBFB, |
| | CBL, CCND1, CCND2, CCND3, CCNE1, CD33, CD52 (CDW52), CD79A, CD79B, |
| | CDC73, CDH1, CDK12, CDK2, CDK4, CDK6, CDK8, CDKN1B, CDKN2A, CDKN2B, |
| | CDKN2C, CEBPA, CES2, CHEK1, CHEK2, CIC, CREBBP, CRKL, CRLF2, CSF1R, |
| | CTCF, CTNNA1, CTNNB1, DAXX, DCK, DDR2, DHFR, DNMT1, DNMT3A, DNMT3B, |
| | DOT1L, EGFR, EMSY (C11orf30), EP300, EPHA2, EPHA3, EPHA5, EPHB1, ERBB2, |
| | ERBB3, ERBB4, ERBB2 (typo?), ERCC3, EREG, ERG, ESR1, ETV1, ETV4, ETV5, ETV6, |
| | EWSR1, EZH2, FAM123B (WTX), FAM46C, FANCA, FANCC, FANCD2, FANCE, |
| | FANCF, FANCG, FANCL, FBXW7, FGF10, FGF14, FGF19, FGF23, FGF3, FGF4, FGF6, |
| | FGFR1, FGFR2, FGFR3, FGFR4, FLT1, FLT3, FLT4, FOLR1, FOLR2, FOXL2, FSHB, |
| | FSHPRH1, FSHR, GART, GATA1, GATA2, GATA3, GID4 (C17orf39), GNA11, GNA13, |
| | GNAQ, GNAS, GNRH1, GNRHR1, GPR124, GRIN2A, GSK3B, GSTP1, HDAC1, HGF, |
| | HIG1, HNF1A, HRAS, HSPCA (HSP90), IDH1, IDH2, IGF1R, IKBKE, IKZF1, IL13RA1, |
| | IL2, IL2RA (CD25), IL7R, INHBA, IRF4, IRS2, JAK1, JAK2, JAK3, JUN, KAT6A |
| | (MYST3), KDM5A, KDM5C, KDM6A, KDR (VEGFR2), KEAP1, KIT, KLHL6, KRAS, |
| | LCK, LRP1B, LTB, LTBR, MAP2K1, MAP2K2, MAP2K4, MAP3K1, MAPK, MCL1, |
| | MDM2, MDM4, MED12, MEF2B, MEN1, MET, MGMT, MITF, MLH1, MLL, MLL2, |
| | MPL, MRE11A, MS4A1 (CD20), MSH2, MSH6, MTAP, MTOR, MUTYH, MYC, MYCL1, |
| | MYCN, MYD88, NF1, NF2, NFE2L2, NFKB1, NFKB2, NFKBIA, NGF, NKX2-1, |
| | NOTCH1, NOTCH2, NPM1, NRAS, NTRK1, NTRK2, NTRK3, NUP93, ODC1, OGFR, |
| | PAK3, PALB2, PAX5, PBRM1, PDGFC, PDGFRA, PDGFRB, PDK1, PGP, PGR (PR), |
| | PIK3CA, PIK3CG, PIK3R1, PIK3R2, POLA, PPARG, PPARGC1, PPP2R1A, PRDM1, |
| | PRKAR1A, PRKDC, PTCH1, PTEN, PTPN11, RAD50, RAD51, RAF1, RARA, RB1, RET, |
| | RICTOR, RNF43, ROS1, RPTOR, RRM1, RRM2, RRM2B, RUNX1, RXR, RXRB, RXRG, |
| | SETD2, SF3B1, SMAD2, SMAD4, SMARCA4, SMARCB1, SMO, SOCS1, SOX10, SOX2, |
| | SPARC, SPEN, SPOP, SRC, SST, SSTR1, SSTR2, SSTR3, SSTR4, SSTR5, STAG2, |
| | STAT4, STK11, SUFU, TET2, TGFBR2, TK1, TLE3, TMPRSS2, TNF, TNFAIP3, |
| | TNFRSF14, TOP1, TOP2, TOP2A, TOP2B, TP53, TS, TSC1, TSC2, TSHR, TUBB3, TXN, |
| | TYMP, VDR, VEGF (VEGFA), VEGFC, VHL, WISP3, WT1, XDH, XPO1, YES1, ZAP70, |
| | ZNF217, ZNF703 |
| Cytohesions | cytohesin-1 (CYTH1), cytohesin-2 (CYTH2; ARNO), cytohesin-3 (CYTH3; Grp1; ARNO3), |
| | cytohesin-4 (CYTH4) |
| Cancer/Angio | Erb 2, Erb 3, Erb 4, UNC93a, B7H3, MUC1, MUC2, MUC16, MUC17, 5T4, RAGE, VEGF |
| | A, VEGFR2, FLT1, DLL4, Epcam |
| Tissue (Breast) | BIG H3, GCDFP-15, PR(B), GPR 30, CYFRA 21, BRCA 1, BRCA 2, ESR 1, ESR2 |
| Tissue (Prostate) | PSMA, PCSA, PSCA, PSA, TMPRSS2 |
| Inflammation/Immune | MFG-E8, IFNAR, CD40, CD80, MICB, HLA-DRb, IL-17-Ra |
| Common vesicle | HSPA8, CD63, Actb, GAPDH, CD9, CD81, ANXA2, HSP90AA1, ENO1, YWHAZ, |
| markers | PDCD6IP, CFL1, SDCBP, PKN2, MSN, MFGE8, EZR, YWHAG, PGK1, EEF1A1, PPIA, |
| | GLC1F, GK, ANXA6, ANXA1, ALDOA, ACTG1, TPI1, LAMP2, HSP90AB1, DPP4, |
| | YWHAB, TSG101, PFN1, LDHB, HSPA1B, HSPA1A, GSTP1, GNAI2, GDI2, CLTC, |
| | ANXA5, YWHAQ, TUBA1A, THBS1, PRDX1, LDHA, LAMP1, CLU, CD86 |
| Common vesicle | CD63, GAPDH, CD9, CD81, ANXA2, ENO1, SDCBP, MSN, MFGE8, EZR, GK, ANXA1, |
| membrane markers | LAMP2, DPP4, TSG101, HSPA1A, GDI2, CLTC, LAMP1, CD86, ANPEP, TFRC, |
| | SLC3A2, RDX, RAP1B, RAB5C, RAB5B, MYH9, ICAM1, FN1, RAB11B, PIGR, |
| | LGALS3, ITGB1, EHD1, CLIC1, ATP1A1, ARF1, RAP1A, P4HB, MUC1, KRT10, HLA- |
| | A, FLOT1, CD59, C1orf58, BASP1, TACSTD1, STOM |
| Common vesicle | MHC class I, MHC class II, Integrins, Alpha 4 beta 1, Alpha M beta 2, Beta 2, |
| markers | ICAM1/CD54, P-selection, Dipeptidylpeptidase IV/CD26, Aminopeptidase n/CD13, CD151, |
| | CD53, CD37, CD82, CD81, CD9, CD63, Hsp70, Hsp84/90 |
| | Actin, Actin-binding proteins, Tubulin, Annexin I, Annexin II, Annexin IV, Annexin V, |
| | Annexin VI, RAB7/RAP1B/RADGDI, Gi2alpha/14-3-3, CBL/LCK, CD63, GAPDH, CD9, |
| | CD81, ANXA2, ENO1, SDCBP, MSN, MFGE8, EZR, GK, ANXA1, LAMP2, DPP4, |
| | TSG101, HSPA1A, GDI2, CLTC, LAMP1, Cd86, ANPEP, TFRC, SLC3A2, RDX, RAP1B, |
| | RAB5C, RAB5B, MYH9, ICAM1, FN1, RAB11B, PIGR, LGALS3, ITGB1, EHD1, CLIC1, |
| | ATP1A1, ARF1, RAP1A, P4HB, MUC1, KRT10, HLA-A, FLOT1, CD59, C1orf58, BASP1, |
| | TACSTD1, STOM |
| Vesicle markers | A33, a33 n15, AFP, ALA, ALIX, ALP, AnnexinV, APC, ASCA, ASPH (246-260), ASPH |
| | (666-680), ASPH (A-10), ASPH (D01P), ASPH (D03), ASPH (G-20), ASPH (H-300), |
| | AURKA, AURKB, B7H3, B7H4, BCA-225, BCNP, BDNF, BRCA, CA125 (MUC16), CA- |
| | 19-9, C-Bir, CD1.1, CD10, CD174 (Lewis y), CD24, CD44, CD46, CD59 (MEM-43), CD63, |
| | CD66e CEA, CD73, CD81, CD9, CDA, CDAC1 1a2, CEA, C-Erb2, C-erbB2, CRMP-2, |
| | CRP, CXCL12, CYFRA21-1, DLL4, DR3, EGFR, Epcam, EphA2, EphA2 (H-77), ER, |
| | ErbB4, EZH2, FASL, FRT, FRT c.f23, GDF15, GPCR, GPR30, Gro-alpha, HAP, HBD 1, |
| | HBD2, HER 3 (ErbB3), HSP, HSP70, hVEGFR2, iC3b, IL 6 Unc, IL-1B, IL6 Unc, IL6R, |
| | IL8, IL-8, INSIG-2, KLK2, L1CAM, LAMN, LDH, MACC-1, MAPK4, MART-1, MCP-1, |
| | M-CSF, MFG-E8, MIC1, MIF, MIS RII, MMG, MMP26, MMP7, MMP9, MS4A1, MUC1, |
| | MUC1 seq1, MUC1 seq11A, MUC17, MUC2, Ncam, NGAL, NPGP/NPFF2, OPG, OPN, |
| | p53, p53, PA2G4, PBP, PCSA, PDGFRB, PGP9.5, PIM1, PR (B), PRL, PSA, PSMA, |
| | PSME3, PTEN, R5-CD9 Tube 1, Reg IV, RUNX2, SCRN1, seprase, SERPINB3, SPARC, |
| | SPB, SPDEF, SRVN, STAT 3, STEAP1, TF (FL-295), TFF3, TGM2, TIMP-1, TIMP1, |
| | TIMP2, TMEM211, TMPRSS2, TNF-alpha, Trail-R2, Trail-R4, TrKB, TROP2, Tsg 101, |
| | TWEAK, UNC93A, VEGF A, YPSMA-1 |
| Vesicle markers | NSE, TRIM29, CD63, CD151, ASPH, LAMP2, TSPAN1, SNAIL, CD45, CKS1, NSE, |
| | FSHR, OPN, FTH1, PGP9, ANNEXIN 1, SPD, CD81, EPCAM, PTH1R, CEA, CYTO 7, |
| | CCL2, SPA, KRAS, TWIST1, AURKB, MMP9, P27, MMP1, HLA, HIF, CEACAM, |
| | CENPH, BTUB, INTG b4, EGFR, NACC1, CYTO 18, NAP2, CYTO 19, ANNEXIN V, |
| | TGM2, ERB2, BRCA1, B7H3, SFTPC, PNT, NCAM, MS4A1, P53, INGA3, MUC2, SPA, |
| | OPN, CD63, CD9, MUC1, UNCR3, PAN ADH, HCG, TIMP, PSMA, GPCR, RACK1, |
| | PSCA, VEGF, BMP2, CD81, CRP, PRO GRP, B7H3, MUC1, M2PK, CD9, PCSA, PSMA |
| Vesicle markers | TFF3, MS4A1, EphA2, GAL3, EGFR, N-gal, PCSA, CD63, MUC1, TGM2, CD81, DR3, |
| | MACC-1, TrKB, CD24, TIMP-1, A33, CD66 CEA, PRL, MMP9, MMP7, TMEM211, |
| | SCRN1, TROP2, TWEAK, CDACC1, UNC93A, APC, C-Erb, CD10, BDNF, FRT, GPR30, |
| | P53, SPR, OPN, MUC2, GRO-1, tsg 101, GDF15 |
| Vesicle markers | CD9, Erb2, Erb4, CD81, Erb3, MUC16, CD63, DLL4, HLA-Drpe, B7H3, IFNAR, 5T4, |
| | PCSA, MICB, PSMA, MFG-E8, Muc1, PSA, Muc2, Unc93a, VEGFR2, EpCAM, VEGF A, |
| | TMPRSS2, RAGE, PSCA, CD40, Muc17, IL-17-RA, CD80 |
| Benign Prostate | BCMA, CEACAM-1, HVEM, IL-1 R4, IL-10 Rb, Trappin-2, p53, hsa-miR-329, hsa-miR- |
| Hyperplasia (BPH) | 30a, hsa-miR-335, hsa-miR-152, hsa-miR-151-5p, hsa-miR-200a, hsa-miR-145, hsa-miR- |
| | 29a, hsa-miR-106b, hsa-miR-595, hsa-miR-142-5p, hsa-miR-99a, hsa-miR-20b, hsa-miR- |
| | 373, hsa-miR-502-5p, hsa-miR-29b, hsa-miR-142-3p, hsa-miR-663, hsa-miR-423-5p, hsa- |
| | miR-15a, hsa-miR-888, hsa-miR-361-3p, hsa-miR-365, hsa-miR-10b, hsa-miR-199a-3p, hsa- |
| | miR-181a, hsa-miR-19a, hsa-miR-125b, hsa-miR-760, hsa-miR-7a, hsa-miR-671-5p, hsa- |
| | miR-7c, hsa-miR-1979, hsa-miR-103 |
| Metastatic Prostate | hsa-miR-100, hsa-miR-1236, hsa-miR-1296, hsa-miR-141, hsa-miR-146b-5p, hsa-miR-17*, |
| Cancer | hsa-miR-181a, hsa-miR-200b, hsa-miR-20a*, hsa-miR-23a*, hsa-miR-331-3p, hsa-miR-375, |
| | hsa-miR-452, hsa-miR-572, hsa-miR-574-3p, hsa-miR-577, hsa-miR-582-3p, hsa-miR-937, |
| | miR-10a, miR-134, miR-141, miR-200b, miR-30a, miR-32, miR-375, miR-495, miR-564, |
| | miR-570, miR-574-3p, miR-885-3p |
| Metastatic Prostate | hsa-miR-200b, hsa-miR-375, hsa-miR-141, hsa-miR-331-3p, hsa-miR-181a, hsa-miR-574-3p |
| Cancer | |
| Prostate Cancer | hsa-miR-574-3p, hsa-miR-141, hsa-miR-432, hsa-miR-326, hsa-miR-2110, hsa-miR-181a- |
| | 2*, hsa-miR-107, hsa-miR-301a, hsa-miR-484, hsa-miR-625* |
| Metastatic Prostate | hsa-miR-582-3p, hsa-miR-20a*, hsa-miR-375, hsa-miR-200b, hsa-miR-379, hsa-miR-572, |
| Cancer | hsa-miR-513a-5p, hsa-miR-577, hsa-miR-23a*, hsa-miR-1236, hsa-miR-609, hsa-miR-17*, |
| | hsa-miR-130b, hsa-miR-619, hsa-miR-624*, hsa-miR-198 |
| Metastatic Prostate | FOX01A, SOX9, CLNS1A, PTGDS, XPO1, LETMD1, RAD23B, ABCC3, APC, CHES1, |
| Cancer | EDNRA, FRZB, HSPG2, TMPRSS2_ETV1 fusion |
| Prostate Cancer | hsa-let-7b, hsa-miR-107, hsa-miR-1205, hsa-miR-1270, hsa-miR-130b, hsa-miR-141, hsa- |
| | miR-143, hsa-miR-148b*, hsa-miR-150, hsa-miR-154*, hsa-miR-181a*, hsa-miR-181a-2*, |
| | hsa-miR-18a*, hsa-miR-19b-1*, hsa-miR-204, hsa-miR-2110, hsa-miR-215, hsa-miR-217, |
| | hsa-miR-219-2-3p, hsa-miR-23b*, hsa-miR-299-5p, hsa-miR-301a, hsa-miR-301a, hsa-miR- |
| | 326, hsa-miR-331-3p, hsa-miR-365*, hsa-miR-373*, hsa-miR-424, hsa-miR-424*, hsa-miR- |
| | 432, hsa-miR-450a, hsa-miR-451, hsa-miR-484, hsa-miR-497, hsa-miR-517*, hsa-miR-517a, |
| | hsa-miR-518f, hsa-miR-574-3p, hsa-miR-595, hsa-miR-617, hsa-miR-625*, hsa-miR-628-5p, |
| | hsa-miR-629, hsa-miR-634, hsa-miR-769-5p, hsa-miR-93, hsa-miR-96 |
| Prostate Cancer | CD9, PSMA, PCSA, CD63, CD81, B7H3, IL 6, OPG-13, IL6R, PA2G4, EZH2, RUNX2, |
| | SERPINB3, EpCam |
| Prostate Cancer | A33, a33 n15, AFP, ALA, ALIX, ALP, AnnexinV, APC, ASCA, ASPH (246-260), ASPH |
| | (666-680), ASPH (A-10), ASPH (D01P), ASPH (D03), ASPH (G-20), ASPH (H-300), |
| | AURKA, AURKB, B7H3, B7H4, BCA-225, BCNP, BDNF, BRCA, CA125 (MUC16), CA- |
| | 19-9, C-Bir, CD1.1, CD10, CD174 (Lewis y), CD24, CD44, CD46, CD59 (MEM-43), CD63, |
| | CD66e CEA, CD73, CD81, CD9, CDA, CDAC1 1a2, CEA, C-Erb2, C-erbB2, CRMP-2, |
| | CRP, CXCL12, CYFRA21-1, DLL4, DR3, EGFR, Epcam, EphA2, EphA2 (H-77), ER, |
| | ErbB4, EZH2, FASL, FRT, FRT c.f23, GDF15, GPCR, GPR30, Gro-alpha, HAP, HBD 1, |
| | HBD2, HER 3 (ErbB3), HSP, HSP70, hVEGFR2, iC3b, IL 6 Unc, IL-1B, IL6 Unc, IL6R, |
| | IL8, IL-8, INSIG-2, KLK2, L1CAM, LAMN, LDH, MACC-1, MAPK4, MART-1, MCP-1, |
| | M-CSF, MFG-E8, MIC1, MIF, MIS RII, MMG, MMP26, MMPI, MMP9, MS4A1, MUC1, |
| | MUC1 seq1, MUC1 seq11A, MUC17, MUC2, Ncam, NGAL, NPGP/NPFF2, OPG, OPN, |
| | p53, p53, PA2G4, PBP, PCSA, PDGFRB, PGP9.5, PIM1, PR (B), PRL, PSA, PSMA, |
| | PSME3, PTEN, R5-CD9 Tube 1, Reg IV, RUNX2, SCRN1, seprase, SERPINB3, SPARC, |
| | SPB, SPDEF, SRVN, STAT 3, STEAP1, TF (FL-295), TFF3, TGM2, TIMP-1, TIMP1, |
| | TIMP2, TMEM211, TMPRSS2, TNF-alpha, Trail-R2, Trail-R4, TrKB, TROP2, Tsg 101, |
| | TWEAK, UNC93A, VEGF A, YPSMA-1 |
| Prostate Cancer | 5T4, ACTG1, ADAM10, ADAM15, ALDOA, ANXA2, ANXA6, APOA1, ATP1A1, |
| Vesicle Markers | BASP1, C1orf58, C20orf114, C8B, CAPZA1, CAV1, CD151, CD2AP, CD59, CD9, CD9, |
| | CFL1, CFP, CHMP4B, CLTC, COTL1, CTNND1, CTSB, CTSZ, CYCS, DPP4, EEF1A1, |
| | EHD1, ENO1, F11R, F2, F5, FAM125A, FNBP1L, FOLH1, GAPDH, GLB1, GPX3, |
| | HIST1H1C, HIST1H2AB, HSP90AB1, HSPA1B, HSPA8, IGSF8, ITGB1, ITIH3, JUP, |
| | LDHA, LDHB, LUM, LYZ, MFGE8, MGAM, MMP9, MYH2, MYL6B, NME1, NME2, |
| | PABPC1, PABPC4, PACSIN2, PCBP2, PDCD6IP, PRDX2, PSA, PSMA, PSMA1, PSMA2, |
| | PSMA4, PSMA6, PSMA7, PSMB1, PSMB2, PSMB3, PSMB4, PSMB5, PSMB6, PSMB8, |
| | PTGFRN, RPS27A, SDCBP, SERINC5, SH3GL1, SLC3A2, SMPDL3B, SNX9, TACSTD1, |
| | TCN2, THBS1, TPI1, TSG101, TUBB, VDAC2, VPS37B, YWHAG, YWHAQ, YWHAZ |
| Prostate Cancer | FLNA, DCRN, HER 3 (ErbB3), VCAN, CD9, GAL3, CDADC1, GM-CSF, EGFR, RANK, |
| Vesicle Markers | CSA, PSMA, ChickenIgY, B7H3, PCSA, CD63, CD3, MUC1, TGM2, CD81, S100-A4, |
| | MFG-E8, Integrin, NK-2R(C-21), PSA, CD24, TIMP-1, IL6 Unc, PBP, PIM1, CA-19-9, |
| | Trail-R4, MMP9, PRL, EphA2, TWEAK, NY-ESO-1, Mammaglobin, UNC93A, A33, |
| | AURKB, CD41, XAGE-1, SPDEF, AMACR, seprase/FAP, NGAL, CXCL12, FRT, CD66e |
| | CEA, SIM2 (C-15), C-Bir, STEAP, PSIP1/LEDGF, MUC17, hVEGFR2, ERG, MUC2, |
| | ADAM10, ASPH (A-10), CA125, Gro-alpha, Tsg 101, SSX2, Trail-R4 |
| Prostate Cancer | NT5E (CD73), A33, ABL2, ADAM10, AFP, ALA, ALIX, ALPL, AMACR, Apo J/CLU, |
| Vesicle Markers | ASCA, ASPH (A-10), ASPH (D01P), AURKB, B7H3, B7H4, BCNP, BDNF, CA125 |
| | (MUC16), CA-19-9, C-Bir (Flagellin), CD10, CD151, CD24, CD3, CD41, CD44, CD46, |
| | CD59(MEM-43), CD63, CD66e CEA, CD81, CD9, CDA, CDADC1, C-erbB2, CRMP-2, |
| | CRP, CSA, CXCL12, CXCR3, CYFRA21-1, DCRN, DDX-1, DLL4, EGFR, EpCAM, |
| | EphA2, ERG, EZH2, FASL, FLNA, FRT, GAL3, GATA2, GM-CSF, Gro-alpha, HAP, |
| | HER3 (ErbB3), HSP70, HSPB1, hVEGFR2, iC3b, IL-1B, IL6 R, IL6 Unc, IL7 R |
| | alpha/CD127, IL8, INSIG-2, Integrin, KLK2, Label, LAMN, Mammaglobin, M-CSF, MFG- |
| | E8, MIF, MIS RII, MMP7, MMP9, MS4A1, MUC1, MUC17, MUC2, Ncam, NDUFB7, |
| | NGAL, NK-2R(C-21), NY-ESO-1, p53, PBP, PCSA, PDGFRB, PIM1, PRL, PSA, |
| | PSIP1/LEDGF, PSMA, RAGE, RANK, Reg IV, RUNX2, S100-A4, seprase/FAP, |
| | SERPINB3, SIM2 (C-15), SPARC, SPC, SPDEF, SPP1, SSX2, SSX4, STEAP, STEAP4, |
| | TFF3, TGM2, TIMP-1, TMEM211, Trail-R2, Trail-R4, TrKB (poly), Trop2, Tsg 101, |
| | TWEAK, UNC93A, VCAN, VEGF A, wnt-5a(C-16), XAGE, XAGE-1 |
| Prostate Vesicle | ADAM 9, ADAM10, AGR2, ALDOA, ALIX, ANXA1, ANXA2, ANXA4, ARF6, ATP1A3, |
| Membrane | B7H3, BCHE, BCL2L14 (Bcl G), BCNP1, BDKRB2, BDNFCAV1-Caveolin1, CCR2 (CC |
| | chemokine receptor 2, CD192), CCR5 (CC chemokine receptor 5), CCT2 (TCP1-beta), |
| | CD10, CD151, CD166/ALCAM, CD24, CD283/TLR3, CD41, CD46, CD49d (Integrin alpha |
| | 4, ITGA4), CD63, CD81, CD9, CD90/THY1, CDH1, CDH2, CDKN1A cyclin-dependent |
| | kinase inhibitor (p21), CGA gene (coding for the alpha subunit of glycoprotein hormones), |
| | CLDN3—Claudin3, COX2 (PTGS2), CSE1L (Cellular Apoptosis Susceptibility), CXCR3, |
| | Cytokeratin 18, Eag1 (KCNH1), EDIL3 (del-1), EDNRB—Endothelial Receptor Type B, |
| | EGFR, EpoR, EZH2 (enhancer of Zeste Homolog2), EZR, FABP5, |
| | Famesyltransferase/geranylgeranyl diphosphate synthase 1 (GGPS1), Fatty acid synthase |
| | (FASN), FTL (light and heavy), GAL3, GDF15-Growth Differentiation Factor 15, GloI, GM- |
| | CSF, GSTP1, H3F3A, HGF (hepatocyte growth factor), hK2/Kif2a, HSP90AA1, HSPA1A/ |
| | HSP70-1, HSPB1, IGFBP-2, IGFBP-3, IL1alpha, IL-6, IQGAP1, ITGAL (Integrin alpha L |
| | chain), Ki67, KLK1, KLK10, KLK11, KLK12, KLK13, KLK14, KLK15, KLK4, KLK5, |
| | KLK6, KLK7, KLK8, KLK9, Lamp-2, LDH-A, LGALS3BP, LGALS8, MMP 1, MMP 2, |
| | MMP 25, MMP 3, MMP10, MMP-14/MT1-MMP, MMP7, MTA1nAnS, Nav1.7, NKX3-1, |
| | Notch1, NRP1/CD304, PAP (ACPP), PGP, PhIP, PIP3/BPNT1, PKM2, PKP1 |
| | (plakophilin1), PKP3 (plakophilin3), Plasma chromogranin-A (CgA), PRDX2, Prostate |
| | secretory protein (PSP94)/β-Microseminoprotein (MSP)/IGBF, PSAP, PSMA, PSMA1, |
| | PTENPTPN13/PTPL1, RPL19, seprase/FAPSET, SLC3A2/CD98, SRVN, STEAP1, |
| | Syndecan/CD138, TGFB, TGM2, TIMP-1TLR4 (CD284), TLR9 (CD289), TMPRSS1/ |
| | hepsin, TMPRSS2, TNFR1, TNFα, Transferrin receptor/CD71/TRFR, Trop2 (TACSTD2), |
| | TWEAK uPA (urokinase plasminoge activator) degrades extracellular matrix, uPAR (uPA |
| | receptor)/CD87, VEGFR1, VEGFR2 |
| Prostate Vesicle | ADAM 34, ADAM 9, AGR2, ALDOA, ANXA1, ANXA 11, ANXA4, ANXA 7, ANXA2, |
| Markers | ARF6, ATP1A1, ATP1A2, ATP1A3, BCHE, BCL2L14 (Bcl G), BDKRB2, CA215, |
| | CAV1—Caveolin1, CCR2 (CC chemokine receptor 2, CD192), CCR5 (CC chemokine receptor 5), |
| | CCT2 (TCP1-beta), CD166/ALCAM, CD49b (Integrin alpha 2, ITGA4), CD90/THY1, |
| | CDH1, CDH2, CDKN1A cyclin-dependent kinase inhibitor (p21), CGA gene (coding for the |
| | alpha subunit of glycoprotein hormones), CHMP4B, CLDN3—Claudin3, CLSTN1 |
| | (Calsyntenin-1), COX2 (PTGS2), CSE1L (Cellular Apoptosis Susceptibility), Cytokeratin 18, |
| | Eag1 (KCNH1) (plasma membrane-K+-voltage gated channel), EDIL3 (del-1), |
| | EDNRB—Endothelial Receptor Type B, Endoglin/CD105, ENOX2—Ecto-NOX disulphide Thiol |
| | exchanger 2, EPCA-2 Early prostate cancer antigen2, EpoR, EZH2 (enhancer of Zeste |
| | Homolog2), EZR, FABP5, Famesyltransferase/geranylgeranyl diphosphate synthase 1 |
| | (GGPS1), Fatty acid synthase (FASN, plasma membrane protein), FTL (light and heavy), |
| | GDF15-Growth Differentiation Factor 15, GloI, GSTP1, H3F3A, HGF (hepatocyte growth |
| | factor), hK2 (KLK2), HSP90AA1, HSPA1A/HSP70-1, IGFBP-2, IGFBP-3, IL1alpha, IL-6, |
| | IQGAP1, ITGAL (Integrin alpha L chain), Ki67, KLK1, KLK10, KLK11, KLK12, KLK13, |
| | KLK14, KLK15, KLK4, KLKS, KLK6, KLK7, KLK8, KLK9, Lamp-2, LDH-A, |
| | LGALS3BP, LGALS8, MFAP5, MMP 1, MMP 2, MMP 24, MMP 25, MMP 3, MMP10, |
| | MMP-14/MT1-MMP, MTA1, nAnS, Nav1.7, NCAM2—Neural cell Adhesion molecule 2, |
| | NGEP/D-TMPP/IPCA-5/ANO7, NKX3-1, Notch1, NRP1/CD304, PGP, PAP (ACPP), |
| | PCA3—Prostate cancer antigen 3, Pdia3/ERp57, PhIP, phosphatidylethanolamine (PE), PIP3, |
| | PKP1 (plakophilin1), PKP3 (plakophilin3), Plasma chromogranin-A (CgA), PRDX2, Prostate |
| | secretory protein (PSP94)/β-Microseminoprotein (MSP)/IGBF, PSAP, PSMA1, PTEN, |
| | PTGFRN, PTPN13/PTPL1, PKM2, RPL19, SCA-1/ATXN1, SERINC5/TPO1, SET, |
| | SLC3A2/CD98, STEAP1, STEAP-3, SRVN, Syndecan/CD138, TGFB, Tissue Polypeptide |
| | Specific antigen TPS, TLR4 (CD284), TLR9 (CD289), TMPRSS1/hepsin, TMPRSS2, |
| | TNFR1, TNFα, CD283/TLR3, Transferrin receptor/CD71/TRFR, uPA (urokinase plasminoge |
| | activator), uPAR (uPA receptor)/CD87, VEGFR1, VEGFR2 |
| Prostate Cancer | hsa-miR-1974, hsa-miR-27b, hsa-miR-103, hsa-miR-146a, hsa-miR-22, hsa-miR-382, hsa- |
| Treatment | miR-23a, hsa-miR-376c, hsa-miR-335, hsa-miR-142-5p, hsa-miR-221, hsa-miR-142-3p, hsa- |
| | miR-151-3p, hsa-miR-21, hsa-miR-16 |
| Prostate Cancer | let-7d, miR-148a, miR-195, miR-25, miR-26b, miR-329, miR-376c, miR-574-3p, miR-888, |
| | miR-9, miR1204, miR-16-2*, miR-497, miR-588, miR-614, miR-765, miR92b*, miR-938, |
| | let-7f-2*, miR-300, miR-523, miR-525-5p, miR-1182, miR-1244, miR-520d-3p, miR-379, |
| | let-7b, miR-125a-3p, miR-1296, miR-134, miR-149, miR-150, miR-187, miR-32, miR-324- |
| | 3p, miR-324-5p, miR-342-3p, miR-378, miR-378*, miR-384, miR-451, miR-455-3p, miR- |
| | 485-3p, miR-487a, miR-490-3p, miR-502-5p, miR-548a-5p, miR-550, miR-562, miR-593, |
| | miR-593*, miR-595, miR-602, miR-603, miR-654-5p, miR-877*, miR-886-5p, miR-125a-5p, |
| | miR-140-3p, miR-192, miR-196a, miR-2110, miR-212, miR-222, miR-224*, miR-30b*, |
| | miR-499-3p, miR-505* |
| Prostate (PCSA + | miR-182, miR-663, miR-155, mirR-125a-5p, miR-548a-5p, miR-628-5p, miR-517*, miR- |
| cMVs) | 450a, miR-920, hsa-miR-619, miR-1913, miR-224*, miR-502-5p, miR-888, miR-376a, miR- |
| | 542-5p, miR-30b*, miR-1179 |
| Prostate Cancer | miR-183-96-182 cluster (miRs-183, 96 and 182), metal ion transporter such as hZIP1, |
| | SLC39A1, SLC39A2, SLC39A3, SLC39A4, SLC39A5, SLC39A6, SLC39A7, SLC39A8, |
| | SLC39A9, SLC39A10, SLC39A11, SLC39A12, SLC39A13, SLC39A14 |
| Prostate Cancer | RAD23B, FBP1, TNFRSF1A, CCNG2, NOTCH3, ETV1, BID, SIM2, LETMD1, ANXA1, |
| | miR-519d, miR-647 |
| Prostate Cancer | RAD23B, FBP1, TNFRSF1A, NOTCH3, ETV1, BID, SIM2, ANXA1, BCL2 |
| Prostate Cancer | ANPEP, ABL1, PSCA, EFNA1, HSPB1, INMT, TRIP13 |
| Prostate Cancer | E2F3, c-met, pRB, EZH2, e-cad, CAXII, CAIX, HIF-1α, Jagged, PIM-1, hepsin, RECK, |
| | Clusterin, MMP9, MTSP-1, MMP24, MMP15, IGFBP-2, IGFBP-3, E2F4, caveolin, EF-1A, |
| | Kallikrein 2, Kallikrein 3, PSGR |
| Prostate Cancer | A2ML1, BAX, C10orf47, C1orf162, CSDA, EIFC3, ETFB, GABARAPL2, GUK1, GZMH, |
| | HIST1H3B, HLA-A, HSP90AA1, NRGN, PRDX5, PTMA, RABAC1, RABAGAP1L, |
| | RPL22, SAP18, SEPW1, SOX1 |
| Prostate Cancer | NY-ESO-1, SSX-2, SSX-4, XAGE-lb, AMACR, p90 autoantigen, LEDGF |
| Prostate Cancer | A33, ABL2, ADAM10, AFP, ALA, ALIX, ALPL, ApoJ/CLU, ASCA, ASPH(A-10), |
| | ASPH(D01P), AURKB, B7H3, B7H3, B7H4, BCNP, BDNF, CA125(MUC16), CA-19-9, C- |
| | Bir, CD10, CD151, CD24, CD41, CD44, CD46, CD59(MEM-43), CD63, CD63, |
| | CD66eCEA, CD81, CD81, CD9, CD9, CDA, CDADC1, CRMP-2, CRP, CXCL12, CXCR3, |
| | CYFRA21-1, DDX-1, DLL4, DLL4, EGFR, Epcam, EphA2, ErbB2, ERG, EZH2, FASL, |
| | FLNA, FRT, GAL3, GATA2, GM-CSF, Gro-alpha, HAP, HER3(ErbB3), HSP70, HSPB1, |
| | hVEGFR2, iC3b, IL-1B, IL6R, IL6Unc, IL7Ralpha/CD127, IL8, INSIG-2, Integrin, KLK2, |
| | LAMN, Mammoglobin, M-CSF, MFG-E8, MIF, MISRII, MMPI, MMP9, MUC1, Muc1, |
| | MUC17, MUC2, Ncam, NDUFB7, NGAL, NK-2R(C-21), NT5E (CD73), p53, PBP, PCSA, |
| | PCSA, PDGFRB, PIM1, PRL, PSA, PSA, PSMA, PSMA, RAGE, RANK, RegIV, RUNX2, |
| | S100-A4, seprase/FAP, SERPINB3, SIM2(C-15), SPARC, SPC, SPDEF, SPP1, STEAP, |
| | STEAP4, TFF3, TGM2, TIMP-1, TMEM211, Trail-R2, Trail-R4, TrKB(poly), Trop2, |
| | Tsg101, TWEAK, UNC93A, VEGFA, wnt-5a(C-16) |
| Prostate Vesicles | CD9, CD63, CD81, PCSA, MUC2, MFG-E8 |
| Prostate Cancer | miR-148a, miR-329, miR-9, miR-378*, miR-25, miR-614, miR-518c*, miR-378, miR-765, |
| | let-7f-2*, miR-574-3p, miR-497, miR-32, miR-379, miR-520g, miR-542-5p, miR-342-3p, |
| | miR-1206, miR-663, miR-222 |
| Prostate Cancer | hsa-miR-877*, hsa-miR-593, hsa-miR-595, hsa-miR-300, hsa-miR-324-5p, hsa-miR-548a- |
| | 5p, hsa-miR-329, hsa-miR-550, hsa-miR-886-5p, hsa-miR-603, hsa-miR-490-3p, hsa-miR- |
| | 938, hsa-miR-149, hsa-miR-150, hsa-miR-1296, hsa-miR-384, hsa-miR-487a, hsa-miRPlus- |
| | C1089, hsa-miR-485-3p, hsa-miR-525-5p |
| Prostate Cancer | hsa-miR-451, hsa-miR-223, hsa-miR-593*, hsa-miR-1974, hsa-miR-486-5p, hsa-miR-19b, |
| | hsa-miR-320b, hsa-miR-92a, hsa-miR-21, hsa-miR-675*, hsa-miR-16, hsa-miR-876-5p, hsa- |
| | miR-144, hsa-miR-126, hsa-miR-137, hsa-miR-1913, hsa-miR-29b-1*, hsa-miR-15a, hsa- |
| | miR-93, hsa-miR-1266 |
| Inflammatory | miR-588, miR-1258, miR-16-2*, miR-938, miR-526b, miR-92b*, let-7d, miR-378*, miR- |
| Disease | 124, miR-376c, miR-26b, miR-1204, miR-574-3p, miR-195, miR-499-3p, miR-2110, miR- |
| | 888 |
| Prostate Cancer | A33, ADAM10, AMACR, ASPH (A-10), AURKB, B7H3, CA125, CA-19-9, C-Bir, CD24, |
| | CD3, CD41, CD63, CD66e CEA, CD81, CD9, CDADC1, CSA, CXCL12, DCRN, EGFR, |
| | EphA2, ERG, FLNA, FRT, GAL3, GM-CSF, Gro-alpha, HER 3 (ErbB3), hVEGFR2, IL6 |
| | Unc, Integrin, Mammaglobin, MFG-E8, MMP9, MUC1, MUC17, MUC2, NGAL, NK-2R(C- |
| | 21), NY-ESO-1, PBP, PCSA, PIM1, PRL, PSA, PSIP1/LEDGF, PSMA, RANK, S100-A4, |
| | seprase/FAP, SIM2 (C-15), SPDEF, SSX2, STEAP, TGM2, TIMP-1, Trail-R4, Tsg 101, |
| | TWEAK, UNC93A, VCAN, XAGE-1 |
| Prostate Cancer | A33, ADAM10, ALIX, AMACR, ASCA, ASPH (A-10), AURKB, B7H3, BCNP, CA125, |
| | CA-19-9, C-Bir (Flagellin), CD24, CD3, CD41, CD63, CD66e CEA, CD81, CD9, CDADC1, |
| | CRP, CSA, CXCL12, CYFRA21-1, DCRN, EGFR, EpCAM, EphA2, ERG, FLNA, GAL3, |
| | GATA2, GM-CSF, Gro alpha, HER3 (ErbB3), HSP70, hVEGFR2, iC3b, IL-1B, IL6 Unc, |
| | IL8, Integrin, KLK2, Mammaglobin, MFG-E8, MMP7, MMP9, MS4A1, MUC1, MUC17, |
| | MUC2, NGAL, NK-2R(C-21), NY-ESO-1, p53, PBP, PCSA, PIM1, PRL, PSA, PSMA, |
| | RANK, RUNX2, S100-A4, seprase/FAP, SERPINB3, SIM2 (C-15), SPC, SPDEF, SSX2, |
| | SSX4, STEAP, TGM2, TIMP-1, TRAIL R2, Trail-R4, Tsg 101, TWEAK, VCAN, VEGF A, |
| | XAGE |
| Prostate Vesicles | EpCam, CD81, PCSA, MUC2, MFG-E8 |
| Prostate Vesicles | CD9, CD63, CD81, MMP7, EpCAM |
| Prostate Cancer | let-7d, miR-148a, miR-195, miR-25, miR-26b, miR-329, miR-376c, miR-574-3p, miR-888, |
| | miR-9, miR1204, miR-16-2*, miR-497, miR-588, miR-614, miR-765, miR92b*, miR-938, |
| | let-7f-2*, miR-300, miR-523, miR-525-5p, miR-1182, miR-1244, miR-520d-3p, miR-379, |
| | let-7b, miR-125a-3p, miR-1296, miR-134, miR-149, miR-150, miR-187, miR-32, miR-324- |
| | 3p, miR-324-5p, miR-342-3p, miR-378, miR-378*, miR-384, miR-451, miR-455-3p, miR- |
| | 485-3p, miR-487a, miR-490-3p, miR-502-5p, miR-548a-5p, miR-550, miR-562, miR-593, |
| | miR-593*, miR-595, miR-602, miR-603, miR-654-5p, miR-877*, miR-886-5p, miR-125a-5p, |
| | miR-140-3p, miR-192, miR-196a, miR-2110, miR-212, miR-222, miR-224*, miR-30b*, |
| | miR-499-3p, miR-505* |
| Prostate Cancer | STAT3, EZH2, p53, MACC1, SPDEF, RUNX2, YB-1, AURKA, AURKB |
| Prostate Cancer | E.001036, E.001497, E.001561, E.002330, E.003402, E.003756, E.004838, E.005471, |
| (Ensembl ENSG | E.005882, E.005893, E.006210, E.006453, E.006625, E.006695, E.006756, E.007264, |
| identifiers) | E.007952, E.008118, E.008196, E.009694, E.009830, E.010244, E.010256, E.010278, |
| | E.010539, E.010810, E.011052, E.011114, E.011143, E.011304, E.011451, E.012061, |
| | E.012779, E.014216, E.014257, E.015133, E.015171, E.015479, E.015676, E.016402, |
| | E.018189, E.018699, E.020922, E.022976, E.023909, E.026508, E.026559, E.029363, |
| | E.029725, E.030582, E.033030, E.035141, E.036257, E.036448, E.038002, E.039068, |
| | E.039560, E.041353, E.044115, E.047410, E.047597, E.048544, E.048828, E.049239, |
| | E.049246, E.049883, E.051596, E.051620, E.052795, E.053108, E.054118, E.054938, |
| | E.056097, E.057252, E.057608, E.058729, E.059122, E.059378, E.059691, E.060339, |
| | E.060688, E.061794, E.061918, E.062485, E.063241, E.063244, E.064201, E.064489, |
| | E.064655, E.064886, E.065054, E.065057, E.065308, E.065427, E.065457, E.065485, |
| | E.065526, E.065548, E.065978, E.066455, E.066557, E.067248, E.067369, E.067704, |
| | E.068724, E.068885, E.069535, E.069712, E.069849, E.069869, E.069956, E.070501, |
| | E.070785, E.070814, E.071246, E.071626, E.071859, E.072042, E.072071, E.072110, |
| | E.072506, E.073050, E.073350, E.073584, E.073756, E.074047, E.074071, E.074964, |
| | E.075131, E.075239, E.075624, E.075651, E.075711, E.075856, E.075886, E.076043, |
| | E.076248, E.076554, E.076864, E.077097, E.077147, E.077312, E.077514, E.077522, |
| | E.078269, E.078295, E.078808, E.078902, E.079246, E.079313, E.079785, E.080572, |
| | E.080823, E.081087, E.081138, E.081181, E.081721, E.081842, E.082212, E.082258, |
| | E.082556, E.083093, E.083720, E.084234, E.084463, E.085224, E.085733, E.086062, |
| | E.086205, E.086717, E.087087, E.087301, E.088888, E.088899, E.088930, E.088992, |
| | E.089048, E.089127, E.089154, E.089177, E.089248, E.089280, E.089902, E.090013, |
| | E.090060, E.090565, E.090612, E.090615, E.090674, E.090861, E.090889, E.091140, |
| | E.091483, E.091542, E.091732, E.092020, E.092199, E.092421, E.092621, E.092820, |
| | E.092871, E.092978, E.093010, E.094755, E.095139, E.095380, E.095485, E.095627, |
| | E.096060, E.096384, E.099331, E.099715, E.099783, E.099785, E.099800, E.099821, |
| | E.099899, E.099917, E.099956, E.100023, E.100056, E.100065, E.100084, E.100142, |
| | E.100191, E.100216, E.100242, E.100271, E.100284, E.100299, E.100311, E.100348, |
| | E.100359, E.100393, E.100399, E.100401, E.100412, E.100442, E.100575, E.100577, |
| | E.100583, E.100601, E.100603, E.100612, E.100632, E.100714, E.100739, E.100796, |
| | E.100802, E.100815, E.100823, E.100836, E.100883, E.101057, E.101126, E.101152, |
| | E.101222, E.101246, E.101265, E.101365, E.101439, E.101557, E.101639, E.101654, |
| | E.101811, E.101812, E.101901, E.102030, E.102054, E.102103, E.102158, E.102174, |
| | E.102241, E.102290, E.102316, E.102362, E.102384, E.102710, E.102780, E.102904, |
| | E.103035, E.103067, E.103175, E.103194, E.103449, E.103479, E.103591, E.103599, |
| | E.103855, E.103978, E.104064, E.104067, E.104131, E.104164, E.104177, E.104228, |
| | E.104331, E.104365, E.104419, E.104442, E.104611, E.104626, E.104723, E.104760, |
| | E.104805, E.104812, E.104823, E.104824, E.105127, E.105220, E.105221, E.105281, |
| | E.105379, E.105402, E.105404, E.105409, E.105419, E.105428, E.105486, E.105514, |
| | E.105518, E.105618, E.105705, E.105723, E.105939, E.105948, E.106049, E.106078, |
| | E.106128, E.106153, E.106346, E.106392, E.106554, E.106565, E.106603, E.106633, |
| | E.107104, E.107164, E.107404, E.107485, E.107551, E.107581, E.107623, E.107798, |
| | E.107816, E.107833, E.107890, E.107897, E.107968, E.108296, E.108312, E.108375, |
| | E.108387, E.108405, E.108417, E.108465, E.108561, E.108582, E.108639, E.108641, |
| | E.108848, E.108883, E.108953, E.109062, E.109184, E.109572, E.109625, E.109758, |
| | E.109790, E.109814, E.109846, E.109956, E.110063, E.110066, E.110104, E.110107, |
| | E.110321, E.110328, E.110921, E.110955, E.111057, E.111218, E.111261, E.111335, |
| | E.111540, E.111605, E.111647, E.111785, E.111790, E.111801, E.111907, E.112039, |
| | E.112081, E.112096, E.112110, E.112144, E.112232, E.112234, E.112473, E.112578, |
| | E.112584, E.112715, E.112941, E.113013, E.113163, E.113282, E.113368, E.113441, |
| | E.113448, E.113522, E.113580, E.113645, E.113719, E.113739, E.113790, E.114054, |
| | E.114127, E.114302, E.114331, E.114388, E.114491, E.114861, E.114867, E.115053, |
| | E.115221, E.115234, E.115239, E.115241, E.115257, E.115339, E.115540, E.115541, |
| | E.115561, E.115604, E.115648, E.115738, E.115758, E.116044, E.116096, E.116127, |
| | E.116254, E.116288, E.116455, E.116478, E.116604, E.116649, E.116726, E.116754, |
| | E.116833, E.117298, E.117308, E.117335, E.117362, E.117411, E.117425, E.117448, |
| | E.117480, E.117592, E.117593, E.117614, E.117676, E.117713, E.117748, E.117751, |
| | E.117877, E.118181, E.118197, E.118260, E.118292, E.118513, E.118523, E.118640, |
| | E.118898, E.119121, E.119138, E.119318, E.119321, E.119335, E.119383, E.119421, |
| | E.119636, E.119681, E.119711, E.119820, E.119888, E.119906, E.120159, E.120328, |
| | E.120337, E.120370, E.120656, E.120733, E.120837, E.120868, E.120915, E.120948, |
| | E.121022, E.121057, E.121068, E.121104, E.121390, E.121671, E.121690, E.121749, |
| | E.121774, E.121879, E.121892, E.121903, E.121940, E.121957, E.122025, E.122033, |
| | E.122126, E.122507, E.122566, E.122705, E.122733, E.122870, E.122884, E.122952, |
| | E.123066, E.123080, E.123143, E.123154, E.123178, E.123416, E.123427, E.123595, |
| | E.123901, E.123908, E.123983, E.123992, E.124143, E.124164, E.124181, E.124193, |
| | E.124216, E.124232, E.124529, E.124562, E.124570, E.124693, E.124749, E.124767, |
| | E.124788, E.124795, E.124831, E.124942, E.125246, E.125257, E.125304, E.125352, |
| | E.125375, E.125445, E.125492, E.125676, E.125753, E.125798, E.125844, E.125868, |
| | E.125901, E.125944, E.125995, E.126062, E.126267, E.126653, E.126773, E.126777, |
| | E.126814, E.126858, E.126883, E.126934, E.126945, E.126952, E.127022, E.127328, |
| | E.127329, E.127399, E.127415, E.127554, E.127616, E.127720, E.127824, E.127884, |
| | E.127914, E.127946, E.127948, E.128050, E.128311, E.128342, E.128609, E.128626, |
| | E.128683, E.128708, E.128881, E.129315, E.129351, E.129355, E.129514, E.129636, |
| | E.129657, E.129757, E.129810, E.129990, E.130175, E.130177, E.130193, E.130255, |
| | E.130299, E.130305, E.130338, E.130340, E.130402, E.130413, E.130612, E.130713, |
| | E.130764, E.130770, E.130810, E.130826, E.130935, E.131351, E.131467, E.131473, |
| | E.131771, E.131773, E.132002, E.132275, E.132323, E.132382, E.132475, E.132481, |
| | E.132589, E.132646, E.132716, E.132881, E.133313, E.133315, E.133687, E.133835, |
| | E.133863, E.133874, E.133961, E.134077, E.134138, E.134207, E.134248, E.134308, |
| | E.134444, E.134452, E.134548, E.134684, E.134759, E.134809, E.134851, E.134955, |
| | E.135052, E.135297, E.135298, E.135387, E.135390, E.135476, E.135486, E.135525, |
| | E.135597, E.135679, E.135740, E.135829, E.135842, E.135870, E.135900, E.135914, |
| | E.135926, E.135940, E.135999, E.136044, E.136068, E.136152, E.136169, E.136280, |
| | E.136371, E.136383, E.136450, E.136521, E.136527, E.136574, E.136710, E.136750, |
| | E.136807, E.136874, E.136875, E.136930, E.136933, E.136935, E.137055, E.137124, |
| | E.137312, E.137409, E.137497, E.137513, E.137558, E.137601, E.137727, E.137776, |
| | E.137806, E.137814, E.137815, E.137948, E.137955, E.138028, E.138031, E.138041, |
| | E.138050, E.138061, E.138069, E.138073, E.138095, E.138160, E.138294, E.138347, |
| | E.138363, E.138385, E.138587, E.138594, E.138621, E.138674, E.138756, E.138757, |
| | E.138760, E.138772, E.138796, E.139211, E.139405, E.139428, E.139517, E.139613, |
| | E.139626, E.139684, E.139697, E.139874, E.140263, E.140265, E.140326, E.140350, |
| | E.140374, E.140382, E.140451, E.140481, E.140497, E.140632, E.140678, E.140694, |
| | E.140743, E.140932, E.141002, E.141012, E.141258, E.141378, E.141425, E.141429, |
| | E.141522, E.141543, E.141639, E.141744, E.141873, E.141994, E.142025, E.142208, |
| | E.142515, E.142606, E.142698, E.142765, E.142864, E.142875, E.143013, E.143294, |
| | E.143321, E.143353, E.143374, E.143375, E.143390, E.143578, E.143614, E.143621, |
| | E.143633, E.143771, E.143797, E.143816, E.143889, E.143924, E.143933, E.143947, |
| | E.144136, E.144224, E.144306, E.144381, E.144410, E.144485, E.144566, E.144671, |
| | E.144741, E.144935, E.145020, E.145632, E.145741, E.145833, E.145888, E.145907, |
| | E.145908, E.145919, E.145990, E.146067, E.146070, E.146281, E.146433, E.146457, |
| | E.146535, E.146701, E.146856, E.146966, E.147044, E.147127, E.147130, E.147133, |
| | E.147140, E.147231, E.147257, E.147403, E.147475, E.147548, E.147697, E.147724, |
| | E.148158, E.148396, E.148488, E.148672, E.148737, E.148835, E.149182, E.149218, |
| | E.149311, E.149480, E.149548, E.149646, E.150051, E.150593, E.150961, E.150991, |
| | E.151092, E.151093, E.151247, E.151304, E.151491, E.151690, E.151715, E.151726, |
| | E.151779, E.151806, E.152086, E.152207, E.152234, E.152291, E.152359, E.152377, |
| | E.152409, E.152422, E.152582, E.152763, E.152818, E.152942, E.153113, E.153140, |
| | E.153391, E.153904, E.153936, E.154099, E.154127, E.154380, E.154639, E.154723, |
| | E.154781, E.154832, E.154864, E.154889, E.154957, E.155368, E.155380, E.155508, |
| | E.155660, E.155714, E.155959, E.155980, E.156006, E.156194, E.156282, E.156304, |
| | E.156467, E.156515, E.156603, E.156650, E.156735, E.156976, E.157064, E.157103, |
| | E.157502, E.157510, E.157538, E.157551, E.157637, E.157764, E.157827, E.157992, |
| | E.158042, E.158290, E.158321, E.158485, E.158545, E.158604, E.158669, E.158715, |
| | E.158747, E.158813, E.158863, E.158901, E.158941, E.158987, E.159147, E.159184, |
| | E.159348, E.159363, E.159387, E.159423, E.159658, E.159692, E.159761, E.159921, |
| | E.160049, E.160226, E.160285, E.160294, E.160633, E.160685, E.160691, E.160789, |
| | E.160862, E.160867, E.160948, E.160972, E.161202, E.161267, E.161649, E.161692, |
| | E.161714, E.161813, E.161939, E.162069, E.162298, E.162385, E.162437, E.162490, |
| | E.162613, E.162641, E.162694, E.162910, E.162975, E.163041, E.163064, E.163110, |
| | E.163257, E.163468, E.163492, E.163530, E.163576, E.163629, E.163644, E.163749, |
| | E.163755, E.163781, E.163825, E.163913, E.163923, E.163930, E.163932, E.164045, |
| | E.164051, E.164053, E.164163, E.164244, E.164270, E.164300, E.164309, E.164442, |
| | E.164488, E.164520, E.164597, E.164749, E.164754, E.164828, E.164916, E.164919, |
| | E.164924, E.165084, E.165119, E.165138, E.165215, E.165259, E.165264, E.165280, |
| | E.165359, E.165410, E.165496, E.165637, E.165646, E.165661, E.165688, E.165695, |
| | E.165699, E.165792, E.165807, E.165813, E.165898, E.165923, E.165934, E.166263, |
| | E.166266, E.166329, E.166337, E.166341, E.166484, E.166526, E.166596, E.166598, |
| | E.166710, E.166747, E.166833, E.166860, E.166946, E.166971, E.167004, E.167085, |
| | E.167110, E.167113, E.167258, E.167513, E.167552, E.167553, E.167604, E.167635, |
| | E.167642, E.167658, E.167699, E.167744, E.167751, E.167766, E.167772, E.167799, |
| | E.167815, E.167969, E.167978, E.167987, E.167996, E.168014, E.168036, E.168066, |
| | E.168071, E.168148, E.168298, E.168393, E.168575, E.168653, E.168746, E.168763, |
| | E.168769, E.168803, E.168916, E.169087, E.169093, E.169122, E.169189, E.169213, |
| | E.169242, E.169410, E.169418, E.169562, E.169592, E.169612, E.169710, E.169763, |
| | E.169789, E.169807, E.169826, E.169957, E.170017, E.170027, E.170037, E.170088, |
| | E.170144, E.170275, E.170310, E.170315, E.170348, E.170374, E.170381, E.170396, |
| | E.170421, E.170430, E.170445, E.170549, E.170632, E.170703, E.170743, E.170837, |
| | E.170854, E.170906, E.170927, E.170954, E.170959, E.171121, E.171155, E.171180, |
| | E.171202, E.171262, E.171302, E.171345, E.171428, E.171488, E.171490, E.171492, |
| | E.171540, E.171643, E.171680, E.171723, E.171793, E.171861, E.171953, E.172115, |
| | E.172283, E.172345, E.172346, E.172466, E.172590, E.172594, E.172653, E.172717, |
| | E.172725, E.172733, E.172831, E.172867, E.172893, E.172939, E.173039, E.173230, |
| | E.173366, E.173473, E.173540, E.173585, E.173599, E.173714, E.173726, E.173805, |
| | E.173809, E.173826, E.173889, E.173898, E.173905, E.174021, E.174100, E.174332, |
| | E.174842, E.174996, E.175063, E.175110, E.175166, E.175175, E.175182, E.175198, |
| | E.175203, E.175216, E.175220, E.175334, E.175416, E.175602, E.175866, E.175946, |
| | E.176102, E.176105, E.176155, E.176171, E.176371, E.176515, E.176900, E.176971, |
| | E.176978, E.176994, E.177156, E.177239, E.177354, E.177409, E.177425, E.177459, |
| | E.177542, E.177548, E.177565, E.177595, E.177628, E.177674, E.177679, E.177694, |
| | E.177697, E.177731, E.177752, E.177951, E.178026, E.178078, E.178104, E.178163, |
| | E.178175, E.178187, E.178234, E.178381, E.178473, E.178741, E.178828, E.178950, |
| | E.179091, E.179115, E.179119, E.179348, E.179388, E.179776, E.179796, E.179869, |
| | E.179912, E.179981, E.180035, E.180198, E.180287, E.180318, E.180667, E.180869, |
| | E.180979, E.180998, E.181072, E.181163, E.181222, E.181234, E.181513, E.181523, |
| | E.181610, E.181773, E.181873, E.181885, E.181924, E.182013, E.182054, E.182217, |
| | E.182271, E.182318, E.182319, E.182512, E.182732, E.182795, E.182872, E.182890, |
| | E.182944, E.183048, E.183092, E.183098, E.183128, E.183207, E.183292, E.183431, |
| | E.183520, E.183684, E.183723, E.183785, E.183831, E.183856, E.184007, E.184047, |
| | E.184113, E.184156, E.184254, E.184363, E.184378, E.184470, E.184481, E.184508, |
| | E.184634, E.184661, E.184697, E.184708, E.184735, E.184840, E.184916, E.185043, |
| | E.185049, E.185122, E.185219, E.185359, E.185499, E.185554, E.185591, E.185619, |
| | E.185736, E.185860, E.185896, E.185945, E.185972, E.186198, E.186205, E.186376, |
| | E.186472, E.186575, E.186591, E.186660, E.186814, E.186834, E.186868, E.186889, |
| | E.187097, E.187323, E.187492, E.187634, E.187764, E.187792, E.187823, E.187837, |
| | E.187840, E.188021, E.188171, E.188186, E.188739, E.188771, E.188846, E.189060, |
| | E.189091, E.189143, E.189144, E.189221, E.189283, E.196236, E.196419, E.196436, |
| | E.196497, E.196504, E.196526, E.196591, E.196700, E.196743, E.196796, E.196812, |
| | E.196872, E.196975, E.196993, E.197081, E.197157, E.197217, E.197223, E.197299, |
| | E.197323, E.197353, E.197451, E.197479, E.197746, E.197779, E.197813, E.197837, |
| | E.197857, E.197872, E.197969, E.197976, E.198001, E.198033, E.198040, E.198087, |
| | E.198131, E.198156, E.198168, E.198205, E.198216, E.198231, E.198265, E.198366, |
| | E.198431, E.198455, E.198563, E.198586, E.198589, E.198712, E.198721, E.198732, |
| | E.198783, E.198793, E.198804, E.198807, E.198824, E.198841, E.198951, E.203301, |
| | E.203795, E.203813, E.203837, E.203879, E.203908, E.204231, E.204316, E.204389, |
| | E.204406, E.204560, E.204574 |
| Prostate Markers | E.005893 (LAMP2), E.006756 (ARSD), E.010539 (ZNF200), E.014257 (ACPP), E.015133 |
| (Ensembl ENSG | (CCDC88C), E.018699 (TTC27), E.044115 (CTNNA1), E.048828 (FAM120A), E.051620 |
| identifiers) | (HEBP2), E.056097 (ZFR), E.060339 (CCAR1), E.063241 (ISOC2), E.064489 (MEF2BNB- |
| | MEF2B), E.064886 (CHI3L2), E.066455 (GOLGA5), E.069535 (MAOB), E.072042 |
| | (RDH11), E.072071 (LPHN1), E.074047 (GLI2), E.076248 (UNG), E.076554 (TPD52), |
| | E.077147 (TM9SF3), E.077312 (SNRPA), E.081842 (PCDHA6), E.086717 (PPEF1), |
| | E.088888 (MAVS), E.088930 (XRN2), E.089902 (RCOR1), E.090612 (ZNF268), E.092199 |
| | (HNRNPC), E.095380 (NANS), E.099783 (HNRNPM), E.100191 (SLC5A4), E.100216 |
| | (TOMM22), E.100242 (SUN2), E.100284 (TOM1), E.100401 (RANGAP1), E.100412 |
| | (ACO2), E.100836 (PABPN1), E.102054 (RBBP7), E.102103 (PQBP1), E.103599 (IQCH), |
| | E.103978 (TMEM87A), E.104177 (MYEF2), E.104228 (TRIM35), E.105428 (ZNRF4), |
| | E.105518 (TMEM205), E.106603 (C7orf44; COA1), E.108405 (P2RX1), E.111057 |
| | (KRT18), E.111218 (PRMT8), E.112081 (SRSF3), E.112144 (ICK), E.113013 (HSPA9), |
| | E.113368 (LMNB1), E.115221 (ITGB6), E.116096 (SPR), E.116754 (SRSF11), E.118197 |
| | (DDX59), E.118898 (PPL), E.119121 (TRPM6), E.119711 (ALDH6A1), E.120656 (TAF12), |
| | E.121671 (CRY2), E.121774 (KHDRBS1), E.122126 (OCRL), E.122566 (HNRNPA2B1), |
| | E.123901 (GPR83), E.124562 (SNRPC), E.124788 (ATXN1), E.124795 (DEK), E.125246 |
| | (CLYBL), E.126883 (NUP214), E.127616 (SMARCA4), E.127884 (ECHS1), E.128050 |
| | (PAICS), E.129351 (ILF3), E.129757 (CDKN1C), E.130338 (TULP4), E.130612 |
| | (CYP2G1P), E.131351 (HAUS8), E.131467 (PSME3), E.133315 (MACROD1), E.134452 |
| | (FBXO18), E.134851 (TMEM165), E.135940 (COX5B), E.136169 (SETDB2), E.136807 |
| | (CDK9), E.137727 (ARHGAP20), E.138031 (ADCY3), E.138050 (THUMPD2), E.138069 |
| | (RAB1A), E.138594 (TMOD3), E.138760 (SCARB2), E.138796 (HADH), E.139613 |
| | (SMARCC2), E.139684 (ESD), E.140263 (SORD), E.140350 (ANP32A), E.140632 |
| | (GLYR1), E.142765 (SYTL1), E.143621 (ILF2), E.143933 (CALM2), E.144410 (CPO), |
| | E.147127 (RAB41), E.151304 (SRFBP1), E.151806 (GUF1), E.152207 (CYSLTR2), |
| | E.152234 (ATP5A1), E.152291 (TGOLN2), E.154723 (ATP5J), E.156467 (UQCRB), |
| | E.159387 (IRX6), E.159761 (C16orf86), E.161813 (LARP4), E.162613 (FUBP1), E.162694 |
| | (EXTL2), E.165264 (NDUFB6), E.167113 (COQ4), E.167513 (CDT1), E.167772 |
| | (ANGPTL4), E.167978 (SRRM2), E.168916 (ZNF608), E.169763 (PRYP3), E.169789 |
| | (PRY), E.169807 (PRY2), E.170017 (ALCAM), E.170144 (HNRNPA3), E.170310 (STX8), |
| | E.170954 (ZNF415), E.170959 (DCDC5), E.171302 (CANT1), E.171643 (S100Z), E.172283 |
| | (PRYP4), E.172590 (MRPL52), E.172867 (KRT2), E.173366 (TLR9), E.173599 (PC), |
| | E.177595 (PIDD), E.178473 (UCN3), E.179981 (TSHZ1), E.181163 (NPM1), E.182319 |
| | (Tyrosine-protein kinase SgK223), E.182795 (C1orf116), E.182944 (EWSR1), E.183092 |
| | (BEGAIN), E.183098 (GPC6), E.184254 (ALDH1A3), E.185619 (PCGF3), E.186889 |
| | (TMEM17), E.187837 (HIST1H1C), E.188771 (C11orf34), E.189060 (H1F0), E.196419 |
| | (XRCC6), E.196436 (NPIPL2), E.196504 (PRPF40A), E.196796, E.196993, E.197451 |
| | (HNRNPAB), E.197746 (PSAP), E.198131 (ZNF544), E.198156, E.198732 (SMOC1), |
| | E.198793 (MTOR), E.039068 (CDH1), E.173230 (GOLGB1), E.124193 (SRSF6), E.140497 |
| | (SCAMP2), E.168393 (DTYMK), E.184708 (EIF4ENIF1), E.124164 (VAPB), E.125753 |
| | (VASP), E.118260 (CREB1), E.135052 (GOLM1), E.010244 (ZNF207), E.010278 (CD9), |
| | E.047597 (XK), E.049246 (PER3), E.069849 (ATP1B3), E.072506 (HSD17B10), E.081138 |
| | (CDH7), E.099785 (MARCH2), E.104331 (IMPAD1), E.104812 (GYS1), E.120868 |
| | (APAF1), E.123908 (EIF2C2), E.125492 (BARHL1), E.127328 (RAB3IP), E.127329 |
| | (PTPRB), E.129514 (FOXA1), E.129657 (SEC14L1), E.129990 (SYT5), E.132881 (RSG1), |
| | E.136521 (NDUFB5), E.138347 (MYPN), E.141429 (GALNT1), E.144566 (RAB5A), |
| | E.151715 (TMEM45B), E.152582 (SPEF2), E.154957 (ZNF18), E.162385 (MAGOH), |
| | E.165410 (CFL2), E.168298 (HIST1H1E), E.169418 (NPR1), E.178187 (ZNF454), |
| | E.178741 (COX5A), E.179115 (FARSA), E.182732 (RGS6), E.183431 (SF3A3), E.185049 |
| | (WHSC2), E.196236 (XPNPEP3), E.197217 (ENTPD4), E.197813, E.203301, E.116833 |
| | (NR5A2), E.121057 (AKAP1), E.005471 (ABCB4), E.071859 (FAM50A), E.084234 |
| | (APLP2), E.101222 (SPEF1), E.103175 (WFDC1), E.103449 (SALL1), E.104805 (NUCB1), |
| | E.105514 (RAB3D), E.107816 (LZTS2), E.108375 (RNF43), E.109790 (KLHL5), E.112039 |
| | (FANCE), E.112715 (VEGFA), E.121690 (DEPDC7), E.125352 (RNF113A), E.134548 |
| | (C12orf39), E.136152 (COG3), E.143816 (WNT9A), E.147130 (ZMYM3), E.148396 |
| | (SEC16A), E.151092 (NGLY1), E.151779 (NBAS), E.155508 (CNOT8), E.163755 (HPS3), |
| | E.166526 (ZNF3), E.172733 (PURG), E.176371 (ZSCAN2), E.177674 (AGTRAP), |
| | E.181773 (GPR3), E.183048 (SLC25A10; MRPL12 SLC25A10), E.186376 (ZNF75D), |
| | E.187323 (DCC), E.198712 (MT-CO2), E.203908 (C6orf221; KHDC3L), E.001497 |
| | (LAS1L), E.009694 (ODZ1), E.080572 (CXorf41; PIH1D3), E.083093 (PALB2), E.089048 |
| | (ESF1), E.100065 (CARD10), E.100739 (BDKRB1), E.102904 (TSNAXIP1), E.104824 |
| | (HNRNPL), E.107404 (DVL1), E.110066 (SUV420H1), E.120328 (PCDHB12), E.121903 |
| | (ZSCAN20), E.122025 (FLT3), E.136930 (PSMB7), E.142025 (DMRTC2), E.144136 |
| | (SLC20A1), E.146535 (GNA12), E.147140 (NONO), E.153391 (INO80C), E.164919 |
| | (COX6C), E.171540 (OTP), E.177951 (BET1L), E.179796 (LRRC3B), E.197479 |
| | (PCDHB11), E.198804 (MT-CO1), E.086205 (FOLH1), E.100632 (ERH), E.100796 |
| | (SMEK1), E.104760 (FGL1), E.114302 (PRKAR2A), E.130299 (GTPBP3), E.133961 |
| | (NUMB), E.144485 (HES6), E.167085 (PHB), E.167635 (ZNF146), E.177239 (MAN1B1), |
| | E.184481 (FOXO4), E.188171 (ZNF626), E.189221 (MAOA), E.157637 (SLC38A10), |
| | E.100883 (SRP54), E.105618 (PRPF31), E.119421 (NDUFA8), E.170837 (GPR27), |
| | E.168148 (HIST3H3), E.135525 (MAP7), E.174996 (KLC2), E.018189 (RUFY3), E.183520 |
| | (UTP11L), E.173905 (GOLIM4), E.165280 (VCP), E.022976 (ZNF839), E.059691 |
| | (PET112), E.063244 (U2AF2), E.075651 (PLD1), E.089177 (KIF16B), E.089280 (FUS), |
| | E.094755 (GABRP), E.096060 (FKBP5), E.100023 (PPIL2), E.100359 (SGSM3), E.100612 |
| | (DHRS7), E.104131 (EIF3J), E.104419 (NDRG1), E.105409 (ATP1A3), E.107623 (GDF10), |
| | E.111335 (OAS2), E.113522 (RAD50), E.115053 (NCL), E.120837 (NFYB), E.122733 |
| | (KIAA1045), E.123178 (SPRYD7), E.124181 (PLCG1), E.126858 (RHOT1), E.128609 |
| | (NDUFA5), E.128683 (GAD1), E.130255 (RPL36), E.133874 (RNF122), E.135387 |
| | (CAPRIN1), E.135999 (EPC2), E.136383 (ALPK3), E.139405 (C12orf52), E.141012 |
| | (GALNS), E.143924 (EML4), E.144671 (SLC22A14), E.145741 (BTF3), E.145907 |
| | (G3BP1), E.149311 (ATM), E.153113 (CAST), E.157538 (DSCR3), E.157992 (KRTCAP3), |
| | E.158901 (WFDC8), E.165259 (HDX), E.169410 (PTPN9), E.170421 (KRT8), E.171155 |
| | (C1GALT1C1), E.172831 (CES2), E.173726 (TOMM20), E.176515, E.177565 (TBL1XR1), |
| | E.177628 (GBA), E.179091 (CYC1), E.189091 (SF3B3), E.197299 (BLM), E.197872 |
| | (FAM49A), E.198205 (ZXDA), E.198455 (ZXDB), E.082212 (ME2), E.109956 (B3GAT1), |
| | E.169710 (FASN), E.011304 (PTBP1), E.057252 (SOAT1), E.059378 (PARP12), E.082258 |
| | (CCNT2), E.087301 (TXNDC16), E.100575 (TIMM9), E.101152 (DNAJC5), E.101812 |
| | (H2BFM), E.102384 (CENPI), E.108641 (B9D1), E.119138 (KLF9), E.119820 (YIPF4), |
| | E.125995 (ROMO1), E.132323 (ILKAP), E.134809 (TIMM10), E.134955 (SLC37A2), |
| | E.135476 (ESPL1), E.136527 (TRA2B), E.137776 (SLTM), E.139211 (AMIGO2), E.139428 |
| | (MMAB), E.139874 (SSTR1), E.143321 (HDGF), E.164244 (PRRC1), E.164270 (HTR4), |
| | E.165119 (HNRNPK), E.165637 (VDAC2), E.165661 (QSOX2), E.167258 (CDK12), |
| | E.167815 (PRDX2), E.168014 (C2CD3), E.168653 (NDUFS5), E.168769 (TET2), E.169242 |
| | (EFNA1), E.175334 (BANF1), E.175416 (CLTB), E.177156 (TALDO1), E.180035 (ZNF48), |
| | E.186591 (UBE2H), E.187097 (ENTPD5), E.188739 (RBM34), E.196497 (IPO4), E.197323 |
| | (TRIM33), E.197857 (ZNF44), E.197976 (AKAP17A), E.064201 (TSPAN32), E.088992 |
| | (TESC), E.092421 (SEMA6A), E.100601 (ALKBH1), E.101557 (USP14), E.103035 |
| | (PSMD7), E.106128 (GHRHR), E.115541 (HSPE1), E.121390 (PSPC1), E.124216 (SNAI1), |
| | E.130713 (EXOSC2), E.132002 (DNAJB1), E.139697 (SBNO1), E.140481 (CCDC33), |
| | E.143013 (LMO4), E.145020 (AMT), E.145990 (GFOD1), E.146070 (PLA2G7), E.164924 |
| | (YWHAZ), E.165807 (PPP1R36), E.167751 (KLK2), E.169213 (RAB3B), E.170906 |
| | (NDUFA3), E.172725 (CORO1B), E.174332 (GLIS1), E.181924 (CHCHD8), E.183128 |
| | (CALHM3), E.204560 (DHX16), E.204574 (ABCF1), E.146701 (MDH2), E.198366 |
| | (HIST1H3A), E.081181 (ARG2), E.185896 (LAMP1), E.077514 (POLD3), E.099800 |
| | (TIMM13), E.100299 (ARSA), E.105419 (MEIS3), E.108417 (KRT37), E.113739 (STC2), |
| | E.125868 (DSTN), E.145908 (ZNF300), E.168575 (SLC20A2), E.182271 (TMIGD1), |
| | E.197223 (C1D), E.186834 (HEXIM1), E.001561 (ENPP4), E.011451 (WIZ), E.053108 |
| | (FSTL4), E.064655 (EYA2), E.065308 (TRAM2), E.075131 (TIPIN), E.081087 (OSTM1), |
| | E.092020 (PPP2R3C), E.096384 (HSP90AB1), E.100348 (TXN2), E.100577 (GSTZ1), |
| | E.100802 (C14orf93), E.101365 (IDH3B), E.101654 (RNMT), E.103067 (ESRP2), E.104064 |
| | (GABPB1), E.104823 (ECH1), E.106565 (TMEM176B), E.108561 (C1QBP), E.115257 |
| | (PCSK4), E.116127 (ALMS1), E.117411 (B4GALT2), E.119335 (SET), E.120337 |
| | (TNFSF18), E.122033 (MTIF3), E.122507 (BBS9), E.122870 (BICC1), E.130177 (CDC16), |
| | E.130193 (C8orf55; THEM6), E.130413 (STK33), E.130770 (ATPIF1), E.133687 (TMTC1), |
| | E.136874 (STX17), E.137409 (MTCH1), E.139626 (ITGB7), E.141744 (PNMT), E.145888 |
| | (GLRA1), E.146067 (FAM193B), E.146433 (TMEM181), E.149480 (MTA2), E.152377 |
| | (SPOCK1), E.152763 (WDR78), E.156976 (EIF4A2), E.157827 (FMNL2), E.158485 |
| | (CD1B), E.158863 (FAM160B2), E.161202 (DVL3), E.161714 (PLCD3), E.163064 (EN1), |
| | E.163468 (CCT3), E.164309 (CMYA5), E.164916 (FOXK1), E.165215 (CLDN3), E.167658 |
| | (EEF2), E.170549 (IRX1), E.171680 (PLEKHG5), E.178234 (GALNT11), E.179869 |
| | (ABCA13), E.179912 (R3HDM2), E.180869 (C1orf180), E.180979 (LRRC57), E.182872 |
| | (RBM10), E.183207 (RUVBL2), E.184113 (CLDN5), E.185972 (CCIN), E.189144 |
| | (ZNF573), E.197353 (LYPD2), E.197779 (ZNF81), E.198807 (PAX9), E.100442 (FKBP3), |
| | E.111790 (FGER1OP2), E.136044 (APPL2), E.061794 (MRPS35), E.065427 (KARS), |
| | E.068885 (IFT80), E.104164 (PLDN; BLOC1S6), E.105127 (AKAP8), E.123066 |
| | (MED13L), E.124831 (LRRFIP1), E.125304 (TM9SF2), E.126934 (MAP2K2), E.130305 |
| | (NSUN5), E.135298 (BAI3), E.135900 (MRPL44), E.136371 (MTHFS), E.136574 |
| | (GATA4), E.140326 (CDAN1), E.141378 (PTRH2), E.141543 (EIF4A3), E.150961 |
| | (SEC24D), E.155368 (DBI), E.161649 (CD300LG), E.161692 (DBF4B), E.162437 |
| | (RAVER2), E.163257 (DCAF16), E.163576 (EFHB), E.163781 (TOPBP1), E.163913 |
| | (IFT122), E.164597 (COG5), E.165359 (DDX26B), E.165646 (SLC18A2), E.169592 |
| | (INO80E), E.169957 (ZNF768), E.171492 (LRRC8D), E.171793 (CTPS; CTPS1), E.171953 |
| | (ATPAF2), E.175182 (FAM131A), E.177354 (C10orf71), E.181610 (MRPS23), E.181873 |
| | (IBA57), E.187792 (ZNF70), E.187823 (ZCCHC16), E.196872 (C2orf55; KIAA1211L), |
| | E.198168 (SVIP), E.160633 (SAFB), E.177697 (CD151), E.181072 (CHRM2), E.012779 |
| | (ALOX5), E.065054 (SLC9A3R2), E.074071 (MRPS34), E.100815 (TRIP11), E.102030 |
| | (NAA10), E.106153 (CHCHD2), E.126814 (TRMT5), E.126952 (NXF5), E.136450 |
| | (SRSF1), E.136710 (CCDC115), E.137124 (ALDH1B1), E.143353 (LYPLAL1), E.162490 |
| | (C1orf187; DRAXIN), E.167799 (NUDT8), E.171490 (RSL1D1), E.173826 (KCNH6), |
| | E.173898 (SPTBN2), E.176900 (OR51T1), E.181513 (ACBD4), E.185554 (NXF2), |
| | E.185945 (NXF2B), E.108848 (LUC7L3), E.029363 (BCLAF1), E.038002 (AGA), E.108312 |
| | (UBTF), E.166341 (DCHS1), E.054118 (THRAP3), E.135679 (MDM2), E.166860 |
| | (ZBTB39), E.183684 (THOC4; ALYREF), E.004838 (ZMYND10), E.007264 (MATK), |
| | E.020922 (MRE11A), E.041353 (RAB27B), E.052795 (FNIP2), E.075711 (DLG1), |
| | E.087087 (SRRT), E.090060 (PAPOLA), E.095139 (ARCN1), E.099715 (PCDH11Y), |
| | E.100271 (TTLL1), E.101057 (MYBL2), E.101265 (RASSF2), E.101901 (ALG13), |
| | E.102290 (PCDH11X), E.103194 (USP10), E.106554 (CHCHD3), E.107833 (NPM3), |
| | E.110063 (DCPS), E.111540 (RAB5B), E.113448 (PDE4D), E.115339 (GALNT3), |
| | E.116254 (CHD5), E.117425 (PTCH2), E.117614 (SYF2), E.118181 (RPS25), E.118292 |
| | (C1orf54), E.119318 (RAD23B), E.121022 (COPS5), E.121104 (FAM117A), E.123427 |
| | (METTL21B), E.125676 (THOC2), E.132275 (RRP8), E.137513 (NARS2), E.138028 |
| | (CGREF1), E.139517 (LNX2), E.143614 (GATAD2B), E.143889 (HNRPLL), E.145833 |
| | (DDX46), E.147403 (RPL10), E.148158 (SNX30), E.151690 (MFSD6), E.153904 (DDAH1), |
| | E.154781 (C3orf19), E.156650 (KAT6B), E.158669 (AGPAT6), E.159363 (ATP13A2), |
| | E.163530 (DPPA2), E.164749 (HNF4G), E.165496 (RPL10L), E.165688 (PMPCA), |
| | E.165792 (METTL17), E.166598 (HSP90B1), E.168036 (CTNNB1), E.168746 (C20orf62), |
| | E.170381 (SEMA3E), E.171180 (OR2M4), E.171202 (TMEM126A), E.172594 |
| | (SMPDL3A), E.172653 (C17orf66), E.173540 (GMPPB), E.173585 (CCR9), E.173809 |
| | (TDRD12), E.175166 (PSMD2), E.177694 (NAALADL2), E.178026 (FAM211B; |
| | C22orf36), E.184363 (PKP3), E.187634 (SAMD11), E.203837 (PNLIPRP3), E.169122 |
| | (FAM110B), E.197969 (VPS13A), E.136068 (FLNB), E.075856 (SART3), E.081721 |
| | (DUSP12), E.102158 (MAGT1), E.102174 (PHEX), E.102316 (MAGED2), E.104723 |
| | (TUSC3), E.105939 (ZC3HAV1), E.108883 (EFTUD2), E.110328 (GALNTL4), E.111785 |
| | (RIC8B), E.113163 (COL4A3BP), E.115604 (IL18R1), E.117362 (APH1A), E.117480 |
| | (FAAH), E.124767 (GLO1), E.126267 (COX6B1), E.130175 (PRKCSH), E.135926 |
| | (TMBIM1), E.138674 (SEC31A), E.140451 (PIF1), E.143797 (MBOAT2), E.149646 |
| | (C20orf152), E.157064 (NMNAT2), E.160294 (MCM3AP), E.165084 (C8orf34), E.166946 |
| | (CCNDBP1), E.170348 (TMED10), E.170703 (TTLL6), E.175198 (PCCA), E.180287 |
| | (PLD5), E.183292 (MIR5096), E.187492 (CDHR4), E.188846 (RPL14), E.015479 |
| | (MATR3), E.100823 (APEX1), E.090615 (GOLGA3), E.086062 (B4GALT1), E.138385 |
| | (SSB), E.140265 (ZSCAN29), E.140932 (CMTM2), E.167969 (ECI1), E.135486 |
| | (HNRNPA1), E.137497 (NUMA1), E.181523 (SGSH), E.099956 (SMARCB1), E.049883 |
| | (PTCD2), E.082556 (OPRK1), E.090674 (MCOLN1), E.107164 (FUBP3), E.108582 (CPD), |
| | E.109758 (HGFAC), E.111605 (CPSF6), E.115239 (ASB3), E.121892 (PDS5A), E.125844 |
| | (RRBP1), E.130826 (DKC1), E.132481 (TRIM47), E.135390 (ATP5G2), E.136875 (PRPF4), |
| | E.138621 (PPCDC), E.145632 (PLK2), E.150051 (MKX), E.153140 (CETN3), E.154127 |
| | (UBASH3B), E.156194 (PPEF2), E.163825 (RTP3), E.164053 (ATRIP), E.164442 |
| | (CITED2), E.168066 (SF1), E.170430 (MGMT), E.175602 (CCDC85B), E.177752 (YIPF7), |
| | E.182512 (GLRX5), E.188186 (C7orf59), E.198721 (ECI2), E.204389 (HSPA1A), E.010256 |
| | (UQCRC1), E.076043 (REXO2), E.102362 (SYTL4), E.161939 (C17orf49), E.173039 |
| | (RELA), E.014216 (CAPN1), E.054938 (CHRDL2), E.065526 (SPEN), E.070501 (POLB), |
| | E.078808 (SDF4), E.083720 (OXCT1), E.100084 (HIRA), E.101246 (ARFRP1), E.102241 |
| | (HTATSF1), E.103591 (AAGAB), E.104626 (ERI1), E.105221 (AKT2), E.105402 (NAPA), |
| | E.105705 (SUGP1), E.106346 (USP42), E.108639 (SYNGR2), E.110107 (PRPF19), |
| | E.112473 (SLC39A7), E.113282 (CLINT1), E.115234 (SNX17), E.115561 (CHMP3), |
| | E.119906 (FAM178A), E.120733 (KDM3B), E.125375 (ATP5S), E.125798 (FOXA2), |
| | E.127415 (IDUA), E.129810 (SGOL1), E.132382 (MYBBP1A), E.133313 (CNDP2), |
| | E.134077 (THUMPD3), E.134248 (HBXIP), E.135597 (REPS1), E.137814 (HAUS2), |
| | E.138041 (SMEK2), E.140382 (HMG20A), E.143578 (CREB3L4), E.144224 (UBXN4), |
| | E.144306 (SCRN3), E.144741 (SLC25A26), E.145919 (BOD1), E.146281 (PM20D2), |
| | E.152359 (POC5), E.152409 (JMY), E.154889 (MPPE1), E.157551 (KCNJ15), E.157764 |
| | (BRAF), E.158987 (RAPGEF6), E.162069 (CCDC64B), E.162910 (MRPL55), E.163749 |
| | (CCDC158), E.164045 (CDC25A), E.164300 (SERINC5), E.165898 (ISCA2), E.167987 |
| | (VPS37C), E.168763 (CNNM3), E.170374 (SP7), E.171488 (LRRC8C), E.178381 |
| | (ZFAND2A), E.180998 (GPR137C), E.182318 (ZSCAN22), E.198040 (ZNF84), E.198216 |
| | (CACNA1E), E.198265 (HELZ), E.198586 (TLK1), E.203795 (FAM24A), E.204231 |
| | (RXRB), E.123992 (DNPEP), E.184634 (MED12), E.181885 (CLDN7), E.186660 (ZFP91), |
| | E.126777 (KTN1), E.080823 (MOK), E.101811 (CSTF2), E.124570 (SERPINB6), E.148835 |
| | (TAF5), E.158715 (SLC45A3), E.110955 (ATP5B), E.127022 (CANX), E.142208 (AKT1), |
| | E.128881 (TTBK2), E.147231 (CXorf57), E.006210 (CX3CL1), E.009830 (POMT2), |
| | E.011114 (BTBD7), E.065057 (NTHL1), E.068724 (TTC7A), E.073584 (SMARCE1), |
| | E.079785 (DDX1), E.084463 (WBP11), E.091140 (DLD), E.099821 (POLRMT), E.101126 |
| | (ADNP), E.104442 (ARMC1), E.105486 (LIG1), E.110921 (MVK), E.113441 (LNPEP), |
| | E.115758 (ODC1), E.116726 (PRAMEF12), E.119681 (LTBP2), E.136933 (RABEPK), |
| | E.137815 (RTF1), E.138095 (LRPPRC), E.138294 (MSMB), E.141873 (SLC39A3), |
| | E.142698 (C1orf94), E.143390 (RFX5), E.148488 (ST8SIA6), E.148737 (TCF7L2), |
| | E.151491 (EPS8), E.152422 (XRCC4), E.154832 (CXXC1), E.158321 (AUTS2), E.159147 |
| | (DONSON), E.160285 (LSS), E.160862 (AZGP1), E.160948 (VPS28), E.160972 |
| | (PPP1R16A), E.165934 (CPSF2), E.167604 (NFKBID), E.167766 (ZNF83), E.168803 |
| | (ADAL), E.169612 (FAM103A1), E.171262 (FAM98B), E.172893 (DHCR7), E.173889 |
| | (PHC3), E.176971 (FIBIN), E.177548 (RABEP2), E.179119 (SPTY2D1), E.184378 |
| | (ACTRT3), E.184508 (HDDC3), E.185043 (CIB1), E.186814 (ZSCAN30), E.186868 |
| | (MAPT), E.196812 (ZSCAN16), E.198563 (DDX39B), E.124529 (HIST1H4B), E.141002 |
| | (TCF25), E.174100 (MRPL45), E.109814 (UGDH), E.138756 (BMP2K), E.065457 |
| | (ADAT1), E.105948 (TTC26), E.109184 (DCUN1D4), E.125257 (ABCC4), E.126062 |
| | (TMEM115), E.142515 (KLK3), E.144381 (HSPD1), E.166710 (B2M), E.198824 |
| | (CHAMP1), E.078902 (TOLLIP), E.099331 (MYO9B), E.102710 (FAM48A), E.107485 |
| | (GATA3), E.120948 (TARDBP), E.187764 (SEMA4D), E.103855 (CD276), E.117751 |
| | (PPP1R8), E.173714 (WFIKKN2), E.172115 (CYCS), E.005882 (PDK2), E.007952 (NOX1), |
| | E.008118 (CAMK1G), E.012061 (ERCC1), E.015171 (ZMYND11), E.036257 (CUL3), |
| | E.057608 (GDI2), E.058729 (RIOK2), E.071246 (VASH1), E.073050 (XRCC1), E.073350 |
| | (LLGL2), E.079246 (XRCC5), E.085733 (CTTN), E.091542 (ALKBH5), E.091732 |
| | (ZC3HC1), E.092621 (PHGDH), E.099899 (TRMT2A), E.099917 (MED15), E.101439 |
| | (CST3), E.103479 (RBL2), E.104611 (SH2D4A), E.105281 (SLC1A5), E.106392 |
| | (C1GALT1), E.107104 (KANK1), E.107798 (LIPA), E.108296 (CWC25), E.109572 |
| | (CLCN3), E.112110 (MRPL18), E.113790 (EHHADH), E.115648 (MLPH), E.117308 |
| | (GALE), E.117335 (CD46), E.118513 (MYB), E.118640 (VAMP8), E.119321 (FKBP15), |
| | E.122705 (CLTA), E.123983 (ACSL3), E.124232 (RBPJL), E.125901 (MRPS26), E.127399 |
| | (LRRC61), E.127554 (GFER), E.128708 (HAT1), E.129355 (CDKN2D), E.130340 (SNX9), |
| | E.130935 (NOL11), E.131771 (PPP1R1B), E.133863 (TEX15), E.134207 (SYT6), E.136935 |
| | (GOLGA1), E.141425 (RPRD1A), E.143374 (TARS2), E.143771 (CNIH4), E.146966 |
| | (DENND2A), E.148672 (GLUD1), E.150593 (PDCD4), E.153936 (HS2ST1), E.154099 |
| | (DNAAF1), E.156006 (NAT2), E.156282 (CLDN17), E.158545 (ZC3H18), E.158604 |
| | (TMED4), E.158813 (EDA), E.159184 (HOXB13), E.161267 (BDH1), E.163492 |
| | (CCDC141), E.163629 (PTPN13), E.164163 (ABCE1), E.164520 (RAET1E), E.165138 |
| | (ANKS6), E.165923 (AGBL2), E.166484 (MAPK7), E.166747 (AP1G1), E.166971 |
| | (AKTIP), E.167744 (NTF4), E.168071 (CCDC88B), E.169087 (HSPBAP1), E.170396 |
| | (ZNF804A), E.170445 (HARS), E.170632 (ARMC10), E.170743 (SYT9), E.171428 |
| | (NAT1), E.172346 (CSDC2), E.173805 (HAP1), E.175175 (PPM1E), E.175203 (DCTN2), |
| | E.177542 (SLC25A22), E.177679 (SRRM3), E.178828 (RNF186), E.182013 (PNMAL1), |
| | E.182054 (IDH2), E.182890 (GLUD2), E.184156 (KCNQ3), E.184697 (CLDN6), E.184735 |
| | (DDX53), E.184840 (TMED9), E.185219 (ZNF445), E.186198 (SLC51B), E.186205 |
| | (MOSC1; MARC1), E.189143 (CLDN4), E.196700 (ZNF512B), E.196743 (GM2A), |
| | E.198087 (CD2AP), E.198951 (NAGA), E.204406 (MBD5), E.002330 (BAD), E.105404 |
| | (RABAC1), E.114127 (XRN1), E.117713 (ARID1A), E.123143 (PKN1), E.130764 |
| | (LRRC47), E.131773 (KHDRBS3), E.137806 (NDUFAF1), E.142864 (SERBP1), E.158747 |
| | (NBL1), E.175063 (UBE2C), E.178104 (PDE4DIP), E.186472 (PCLO), E.069956 (MAPK6), |
| | E.112941 (PAPD7), E.116604 (MEF2D), E.142875 (PRKACB), E.147133 (TAF1), |
| | E.157510 (AFAP1L1), E.006625 (GGCT), E.155980 (KIF5A), E.134444 (KIAA1468), |
| | E.107968 (MAP3K8), E.117592 (PRDX6), E.123154 (WDR83), E.135297 (MTO1), |
| | E.135829 (DHX9), E.149548 (CCDC15), E.152086 (TUBA3E), E.167553 (TUBA1C), |
| | E.169826 (CSGALNACT2), E.171121 (KCNMB3), E.198033 (TUBA3C), E.147724 |
| | (FAM135B), E.170854 (MINA), E.006695 (COX10), E.067369 (TP53BP1), E.089248 |
| | (ERP29), E.112096 (SOD2), E.138073 (PREB), E.146856 (AGBL3), E.159423 (ALDH4A1), |
| | E.171345 (KRT19), E.172345 (STARD5), E.111647 (UHRF1BP1L), E.117877 (CD3EAP), |
| | E.155714 (PDZD9), E.156603 (MED19), E.075886 (TUBA3D), E.167699 (GLOD4), |
| | E.121749 (TBC1D15), E.090861 (AARS), E.093010 (COMT), E.117676 (RPS6KA1), |
| | E.157502 (MUM1L1), E.159921 (GNE), E.169562 (GJB1), E.179776 (CDH5), E.071626 |
| | (DAZAP1), E.085224 (ATRX), E.116478 (HDAC1), E.117298 (ECE1), E.176171 (BNIP3), |
| | E.177425 (PAWR), E.179348 (GATA2), E.187840 (EIF4EBP1), E.033030 (ZCCHC8), |
| | E.049239 (H6PD), E.060688 (SNRNP40), E.075239 (ACAT1), E.095627 (TDRD1), |
| | E.109625 (CPZ), E.113719 (ERGIC1), E.126773 (C14orf135; PCNXL4), E.149218 |
| | (ENDOD1), E.162975 (KCNF1), E.183785 (TUBA8), E.198589 (LRBA), E.105379 (ETFB), |
| | E.011052 (NME2), E.011143 (MKS1), E.048544 (MRPS10), E.062485 (CS), E.114054 |
| | (PCCB), E.138587 (MNS1), E.155959 (VBP1), E.181222 (POLR2A), E.183723 (CMTM4), |
| | E.184661 (CDCA2), E.204316 (MRPL38), E.140694 (PARN), E.035141 (FAM136A), |
| | E.095485 (CWF19L1), E.115540 (MOB4), E.123595 (RAB9A), E.140678 (ITGAX), |
| | E.141258 (SGSM2), E.158941 (KIAA1967), E.169189 (NSMCE1), E.198431 (TXNRD1), |
| | E.016402 (IL20RA), E.112234 (FBXL4), E.125445 (MRPS7), E.128342 (LIF), E.164051 |
| | (CCDC51), E.175866 (BAIAP2), E.102780 (DGKH), E.203813 (HIST1H3H), E.198231 |
| | (DDX42), E.030582 (GRN), E.106049 (HIBADH), E.130810 (PPAN), E.132475 (H3F3B), |
| | E.158290 (CUL4B), E.166266 (CUL5), E.026559 (KCNG1), E.059122 (FLYWCH1), |
| | E.107897 (ACBD5), E.121068 (TBX2), E.125944 (HNRNPR), E.134308 (YWHAQ), |
| | E.137558 (PI15), E.137601 (NEK1), E.147548 (WHSC1L1), E.149182 (ARFGAP2), |
| | E.159658 (KIAA0494), E.165699 (TSC1), E.170927 (PKHD1), E.186575 (NF2), E.188021 |
| | (UBQLN2), E.167552 (TUBA1A), E.003756 (RBM5), E.134138 (MEIS2), E.008196 |
| | (TFAP2B), E.079313 (REXO1), E.089127 (OAS1), E.106078 (COBL), E.113645 (WWC1), |
| | E.116288 (PARK7), E.121940 (CLCC1), E.136280 (CCM2), E.141639 (MAPK4), E.147475 |
| | (ERLIN2), E.155660 (PDIA4), E.162298 (SYVN1), E.176978 (DPP7), E.176994 (SMCR8), |
| | E.178175 (ZNF366), E.196591 (HDAC2), E.127824 (TUBA4A), E.163932 (PRKCD), |
| | E.143375 (CGN), E.076864 (RAP1GAP), E.138772 (ANXA3), E.163041 (H3F3A), |
| | E.165813 (C10orf118), E.166337 (TAF10), E.178078 (STAP2), E.184007 (PTP4A2), |
| | E.167004 (PDIA3), E.039560 (RAI14), E.119636 (C14orf45), E.140374 (ETFA), E.143633 |
| | (C1orf131), E.144935 (TRPC1), E.156735 (BAG4), E.159348 (CYB5R1), E.170275 |
| | (CRTAP), E.172717 (FAM71D), E.172939 (OXSR1), E.176105 (YES1), E.078295 |
| | (ADCY2), E.119888 (EPCAM), E.141522 (ARHGDIA), E.184047 (DIABLO), E.109062 |
| | (SLC9A3R1), E.170037 (CNTROB), E.066557 (LRRC40), E.074964 (ARHGEF10L), |
| | E.078269 (SYNJ2), E.090013 (BLVRB), E.100142 (POLR2F), E.100399 (CHADL), |
| | E.104365 (IKBKB), E.111261 (MANSC1), E.111907 (TPD52L1), E.112578 (BYSL), |
| | E.121957 (GPSM2), E.122884 (P4HA1), E.124693 (HIST1H3B), E.126653 (NSRP1), |
| | E.130402 (ACTN4), E.138757 (G3BP2), E.150991 (UBC), E.164828 (SUN1), E.175216 |
| | (CKAP5), E.176155 (CCDC57), E.177459 (C8orf47), E.183856 (IQGAP3), E.185122 |
| | (HSF1), E.122952 (ZWINT), E.151093 (OXSM), E.067704 (IARS2), E.088899 (ProSAP- |
| | interacting protein 1), E.091483 (FH), E.114388 (NPRL2), E.114861 (FOXP1), E.135914 |
| | (HTR2B), E.197837 (HIST4H4), E.127720 (C12orf26; METTL25), E.123416 (TUBA1B), |
| | E.047410 (TPR), E.117748 (RPA2), E.133835 (HSD17B4), E.067248 (DHX29), E.121879 |
| | (PIK3CA), E.132589 (FLOT2), E.136750 (GAD2), E.160789 (LMNA), E.166329, E.170088 |
| | (TMEM192), E.175946 (KLHL38), E.178163 (ZNF518B), E.182217 (HIST2H4B), |
| | E.184470 (TXNRD2), E.110321 (EIF4G2), E.171861 (RNMTL1), E.065978 (YBX1), |
| | E.115738 (ID2), E.143294 (PRCC), E.158042 (MRPL17), E.169093 (ASMTL), E.090565 |
| | (RAB11FIP3), E.185591 (SP1), E.156304 (SCAF4), E.092978 (GPATCH2), E.100056 |
| | (DGCR14), E.100583 (SAMD15), E.105723 (GSK3A), E.107551 (RASSF4), E.107581 |
| | (EIF3A), E.107890 (ANKRD26), E.110104 (CCDC86), E.112584 (FAM120B), E.113580 |
| | (NR3C1), E.114491 (UMPS), E.137312 (FLOT1), E.137955 (RABGGTB), E.141994 |
| | (DUS3L), E.147044 (CASK), E.152818 (UTRN), E.180667 (YOD1), E.184916 (JAG2), |
| | E.196526 (AFAP1), E.198783 (ZNF830), E.108465 (CDK5RAP3), E.156515 (HK1), |
| | E.036448 (MYOM2), E.061918 (GUCY1B3), E.070785 (EIF2B3), E.116044 (NFE2L2), |
| | E.128311 (TST), E.131473 (ACLY), E.132716 (DCAF8), E.138363 (ATIC), E.166596 |
| | (WDR16), E.170027 (YWHAG), E.174021 (GNG5), E.203879 (GDI1), E.160049 (DFFA), |
| | E.010810 (FYN), E.051596 (THOC3), E.006453 (BAI1-associated protein 2-like 1), |
| | E.126945 (HNRNPH2), E.165695 (AK8), E.069869 (NEDD4), E.111801 (BTN3A3), |
| | E.112232 (KHDRBS2), E.128626 (MRPS12), E.129636 (ITFG1), E.137948 (BRDT), |
| | E.147257 (GPC3), E.155380 (SLC16A1), E.159692 (CTBP1), E.166833 (NAV2), E.172466 |
| | (ZNF24), E.175110 (MRPS22), E.176102 (CSTF3), E.179388 (EGR3), E.185359 (HGS), |
| | E.198001 (IRAK4), E.100603 (SNW1), E.162641 (AKNAD1), E.069712 (KIAA1107), |
| | E.073756 (PTGS2), E.077522 (ACTN2), E.101639 (CEP192), E.106633 (GCK), E.115241 |
| | (PPM1G), E.116649 (SRM), E.120370 (GORAB), E.124143 (ARHGAP40), E.127948 |
| | (POR), E.129315 (CCNT1), E.132646 (PCNA), E.135740 (SLC9A5), E.151726 (ACSL1), |
| | E.154380 (ENAH), E.157103 (SLC6A1), E.163930 (BAP1), E.164488 (DACT2), E.164754 |
| | (RAD21), E.175220 (ARHGAP1), E.180318 (ALX1), E.181234 (TMEM132C), E.197081 |
| | (IGF2R), E.092871 (RFFL), E.163644 (PPM1K), E.171723 (GPHN), E.108953 (YWHAE), |
| | E.072110 (ACTN1), E.077097 (TOP2B), E.090889 (KIF4A), E.114331 (ACAP2), E.114867 |
| | (EIF4G1), E.117593 (DARS2), E.118523 (CTGF), E.120915 (EPHX2), E.134759 (ELP2), |
| | E.138061 (CYP1B1), E.140743 (CDR2), E.151247 (EIF4E), E.152942 (RAD17), E.160685 |
| | (ZBTB7B), E.163923 (RPL39L), E.167642 (SPINT2), E.167996 (FTH1), E.185736 |
| | (ADARB2), E.198841 (KTI12), E.185860 (C1orf110), E.160226 (C21orf2), E.070814 |
| | (TCOF1), E.124749 (COL21A1), E.154639 (CXADR), E.065485 (PDIA5), E.023909 |
| | (GCLM), E.100714 (MTHFD1), E.108387 (SEPT4), E.160867 (FGFR4), E.134684 (YARS), |
| | E.123080 (CDKN2C), E.065548 (ZC3H15), E.116455 (WDR77), E.117448 (AKR1A1), |
| | E.100393 (EP300), E.138160 (KIF11), E.166263 (STXBP4), E.173473 (SMARCC1), |
| | E.124942 (AHNAK), E.174842 (GLMN), E.180198 (RCC1), E.185499 (MUC1), E.143947 |
| | (RPS27A), E.170315 (UBB), E.003402 (CFLAR), E.137055 (PLAA), E.142606 (MMEL1), |
| | E.147697 (GSDMC), E.163110 (PDLIM5), E.135842 (FAM129A), E.160691 (SHC1), |
| | E.197157 (SND1), E.029725 (RABEP1), E.127946 (HIP1), E.001036 (FUCA2), E.109846 |
| | (CRYAB), E.183831 (ANKRD45), E.189283 (FHIT), E.092820 (EZR), E.104067 (TJP1), |
| | E.120159 (C9orf82; CAAP1), E.154864 (PIEZO2), E.196975 (ANXA4), E.105220 (GPI), |
| | E.127914 (AKAP9), E.135870 (RC3H1), E.026508 (CD44), E.089154 (GCN1L1), E.100311 |
| | (PDGFB), E.119383 (PPP2R4), E.075624 (ACTB), E.177409 (SAMD9L), E.177731 (FLII), |
| | E.015676 (NUDCD3), E.146457 (WTAP), E.178950 (GAK), E.167110 (GOLGA2) |
| Prostate vesicle | LAMP2, ACPP, CTNNA1, HEBP2, ISOC2, HNRNPC, HNRNPM, TOMM22, TOM1, |
| | ACO2, KRT18, HSPA9, LMNB1, SPR, PPL, ALDH6A1, HNRNPA2B1, ATXN1, |
| | SMARCA4, ECHS1, PAICS, ILF3, PSME3, COX5B, RAB1A, SCARB2, HADH, ESD, |
| | SORD, ILF2, CALM2, ATP5A1, TGOLN2, ANGPTL4, ALCAM, KRT2, PC, NPM1, |
| | C1orf116, GPC6, ALDH1A3, HIST1H1C, XRCC6, HNRNPAB, PSAP, CDH1, SCAMP2, |
| | VASP, CD9, ATP1B3, HSD17B10, APAF1, EIF2C2, RAB5A, CFL2, FARSA, XPNPEP3, |
| | ENTPD4, APLP2, NUCB1, RAB3D, VEGFA, HPS3, TSNAXIP1, HNRNPL, PSMB7, |
| | GNA12, NONO, FOLH1, PRKAR2A, PHB, HIST3H3, MAP7, VCP, U2AF2, FUS, FKBP5, |
| | NDRG1, ATP1A3, NCL, RPL36, KRT8, C1GALT1C1, FASN, PTBP1, TXNDC16, |
| | DNAJC5, SLC37A2, HNRNPK, VDAC2, PRDX2, TALDO1, USP14, PSMD7, HSPE1, |
| | DNAJB1, YWHAZ, RAB3B, CORO1B, MDH2, HIST1H3A, LAMP1, STC2, DSTN, |
| | SLC20A2, ENPP4, WIZ, HSP90AB1, IDH3B, ECH1, C1QBP, SET, TNFSF18, ITGB7, |
| | SPOCK1, EIF4A2, CCT3, CLDN3, EEF2, LRRC57, RUVBL2, CLDN5, APPL2, TM9SF2, |
| | EIF4A3, DBI, DBF4B, SVIP, CD151, ALOX5, SLC9A3R2, RAB27B, DLG1, ARCN1, |
| | CHCHD3, RAB5B, RPS25, RPL10, DDAH1, HSP90B1, CTNNB1, PSMD2, PKP3, FLNB, |
| | EFTUD2, GLO1, PRKCSH, TMBIM1, SEC31A, TMED10, RPL14, MATR3, APEX1, |
| | B4GALT1, HNRNPA1, CPD, HSPA1A, CAPN1, CHRDL2, SPEN, SDF4, NAPA, |
| | SYNGR2, CHMP3, CNDP2, CCDC64B, SERINC5, VPS37C, DNPEP, CLDN7, KTN1, |
| | SERPINB6, ATP5B, CANX, AKT1, TTBK2, DDX1, DLD, LNPEP, LTBP2, LRPPRC, |
| | EPS8, AZGP1, VPS28, DHCR7, CIB1, DDX39B, HIST1H4B, UGDH, HSPD1, B2M, |
| | TOLLIP, CD276, CYCS, CUL3, GDI2, LLGL2, XRCC5, CTTN, PHGDH, CST3, RBL2, |
| | SLC1A5, CD46, VAMP8, CLTA, ACSL3, MRPS26, SNX9, GLUD1, TMED4, PTPN13, |
| | AP1G1, SYT9, DCTN2, IDH2, GLUD2, TMED9, CLDN4, GM2A, CD2AP, MBD5, |
| | SERBP1, NBL1, PRKACB, GGCT, PRDX6, DHX9, TUBA3E, TUBA1C, TUBA3C, |
| | ERP29, SOD2, KRT19, TUBA3D, AARS, COMT, MUM1L1, CDH5, ECE1, ACAT1, |
| | ENDOD1, TUBA8, ETFB, NME2, CS, VBP1, RAB9A, TXNRD1, LIF, BAIAP2, |
| | HIST1H3H, GRN, HIBADH, H3F3B, CUL4B, HNRNPR, YWHAQ, PKHD1, TUBA1A, |
| | PARK7, ERLIN2, PDIA4, TUBA4A, PRKCD, ANXA3, H3F3A, PTP4A2, PDIA3, ETFA, |
| | CYB5R1, CRTAP, OXSR1, YES1, EPCAM, ARHGDIA, DIABLO, SLC9A3R1, BLVRB, |
| | P4HA1, HIST1H3B, ACTN4, UBC, FH, HIST4H4, TUBA1B, HSD17B4, PIK3CA, FLOT2, |
| | LMNA, TMEM192, HIST2H4B, YBX1, EIF3A, FLOT1, UTRN, HK1, ACLY, ATIC, |
| | YWHAG, GNG5, GDI1, HNRNPH2, NEDD4, BTN3A3, SLC16A1, HGS, ACTN2, SRM, |
| | PCNA, ACSL1, RAD21, ARHGAP1, IGF2R, YWHAE, ACTN1, EIF4G1, EPHX2, EIF4E, |
| | FTH1, CXADR, MTHFD1, AKR1A1, STXBP4, AHNAK, MUC1, RPS27A, UBB, |
| | PDLIM5, FAM129A, SND1, FUCA2, CRYAB, EZR, TJP1, ANXA4, GPI, AKAP9, CD44, |
| | GCN1L1, ACTB, FLII, NUDCD3 |
| Prostate Cancer | EGFR, GLUD2, ANXA3, APLP2, BclG, Cofilin 2/cfL2, DCTN-50/DCTN2, DDAH1, |
| vesicles | ESD, FARSLA, GITRL, PRKCSH, SLC20A2, Synaptogyrin 2/SYNGR2, TM9SF2, |
| | Calnexin, TOMM22, NDRG1, RPL10, RPL14, USP14, VDAC2, LLGL2, CD63, CD81, |
| | uPAR/CD87, ADAM 9, BDKRB2, CCR5, CCT2 (TCP1-beta), PSMA, PSMA1, HSPB1, |
| | VAMP8, Rab1A, B4GALT1, Aspartyl Aminopeptidase/Dnpep, ATPase Na+/K+ beta |
| | 3/ATP1B3, BDNF, ATPB, beta 2 Microglobulin, Calmodulin 2/CALM2, CD9, XRCC5/ |
| | Ku80, SMARCA4, TOM1, Cytochrome C, Hsp10/HSPE1, COX2/PTGS2, Claudin 4/ |
| | CLDN4, Cytokeratin 8, Cortactin/CTTN, DBF4B/DRF1, ECH1, ECHS1, GOLPH2, ETS1, |
| | DIP13B/appl2, EZH2/KMT6, GSTP1, hK2/Kif2a, IQGAP1, KLK13, Lamp-2, GM2A, |
| | Hsp40/DNAJB1, HADH/HADHSC, Hsp90B, Nucleophosmin, p130/RBL2, PHGDH, |
| | RAB3B, ANXA1, PSMD7, PTBP1, Rab5a, SCARB2, Stanniocalcin 2/STC2, TGN46/ |
| | TGOLN2, TSNAXIP1, ANXA2, CD46, KLK14, IL1alpha, hnRNP C1 + C2, hnRNP A1, |
| | hnRNP A2B1, Claudin 5, CORO1B, Integrin beta 7, CD41, CD49d, CDH2, COX5b, IDH2, |
| | ME1, PhIP, ALDOA, EDNRB/EDN3, MTA1, NKX3-1, TMPRSS2, CD10, CD24, CDH1, |
| | ADAM10, B7H3, CD276, CHRDL2, SPOCK1, VEGFA, BCHE, CD151, CD166/ALCAM, |
| | CSE1L, GPC6, CXCR3, GAL3, GDF15, IGFBP-2, HGF, KLK12, ITGAL, KLK7, KLK9, |
| | MMP 2, MMP 25, MMP10, TNFRI, Notch1, PAP - same as ACPP, PTPN13/PTPL1, |
| | seprase/FAP, TNFR1, TWEAK, VEGFR2, E-Cadherin, Hsp60, CLDN3—Claudin3, KLK6, |
| | KLK8, EDIL3 (del-1), APE1, MMP 1, MMP3, nAnS, PSP94/MSP/IGBF, PSAP, RPL19, |
| | SET, TGFB, TGM2, TIMP-1, TNFRII, MDH2, PKP1, Cystatin C, Trop2/TACSTD2, CCR2/ |
| | CD192, hnRNP M1-M4, CDKN1A, CGA, Cytokeratin 18, EpoR, GGPS1, FTL (light and |
| | heavy), GM-CSF, HSP90AA1, IDH3B, MKI67/Ki67, LTBP2, KLK1, KLK4, KLK5, LDH- |
| | A, Nav1.7/SCN9A, NRP1/CD304, PIP3/BPNT1, PKP3, CgA, PRDX2, SRVN, ATPase |
| | Na+/K+ alpha 3/ATP1A3, SLC3A2/CD98, U2AF2, TLR4 (CD284), TMPRSS1, TNFα, |
| | uPA, GloI, ALIX, PKM2, FABP5, CAV1, TLR9/CD289, ANXA4, PLEKHC1/Kindlin-2, |
| | CD71/TRFR, MBD5, SPEN/RBM15, LGALS8, SLC9A3R2, ENTPD4, ANGPTL4, p97/ |
| | VCP, TBX5, PTEN, Prohibitin, LSP1, HOXB13, DDX1, AKT1, ARF6, EZR, H3F3A, CIB1, |
| | Ku70 (XRCC6), KLK11, TMBIM6, SYT9, APAF1, CLDN7, MATR3, CD90/THY1, Tollip, |
| | NOTCH4, 14-3-3 zeta/beta, ATP5A1, DLG1, GRP94, FKBP5/FKBP51, LAMP1, |
| | LGALS3BP, GDI2, HSPA1A, NCL, KLK15, Cytokeratin basic, EDN-3, AGR2, KLK10, |
| | BRG1, FUS, Histone H4, hnRNP L, Catenin Alpha 1, hnRNP K (F45)*, MMP7*, DBI*, beta |
| | catenin, CTH, CTNND2, Ataxin 1, Proteasome 20S beta 7, ADE2, EZH2, GSTP1, Lamin |
| | B1, Coatomer Subunit Delta, ERAB, Mortalin, PKM2, IGFBP-3, CTNND1/delta 1-catenin/ |
| | p120-catenin, PKA R2, NONO, Sorbitol Dehydrogenase, Aconitase 2, VASP, Lipoamide |
| | Dehydrogenase, AP1G1, GOLPH2, ALDH6A1, AZGP1, Ago2, CNDP2, Nucleobindin-1, |
| | SerpinB6, RUVBL2, Proteasome 19S 10B, SH3PX1, SPR, Destrin, MDM4, FLNB, FASN, |
| | PSME |
| Prostate Cancer | 14-3-3 zeta/beta, Aconitase 2, ADAM 9, ADAM10, ADE2, AFM, Ago2, AGR2, AKT1, |
| vesicles | ALDH1A3, ALDH6A1, ALDOA, ALIX, ANGPTL4, ANXA1, ANXA2, ANXA3, ANXA3, |
| | ANXA4, AP1G1, APAF1, APE1, APLP2, APLP2, ARF6, Aspartyl Aminopeptidase/Dnpep, |
| | Ataxin 1, ATP5A1, ATPase Na+/K+ alpha 3/ATP1A3, ATPase Na+/K+ beta 3/ATP1B3, |
| | ATPase Na+/K+ beta 3/ATP1B3, ATPB, AZGP1, B4GALT1, B7H3, BCHE, BclG, |
| | BDKRB2, BDNF, BDNF, beta 2 Microglobulin, beta catenin, BRG1, CALM2, Calmodulin 2/ |
| | CALM2, Calnexin, Calpain 1, Catenin Alpha 1, CAV1, CCR2/CD192, CCR5, CCT2 |
| | (TCP1-beta), CD10, CD151, CD166/ALCAM, CD24, CD276, CD41, CD46, CD49d, CD63, |
| | CD71/TRFR, CD81, CD9, CD9, CD90/THY1, CDH1, CDH2, CDKN1A, CGA, CgA, |
| | CHRDL2, CIB1, CIB1, Claudin 4/CLDN4, Claudin 5, CLDN3, CLDN3—Claudin3, CLDN4, |
| | CLDN7, CNDP2, Coatomer Subunit Delta, Cofilin 2/cfL2, CORO1B, Cortactin/CTTN, |
| | COX2/PTGS2, COX5b, CSE1L, CTH, CTNND1/delta 1-catenin/p120-catenin, |
| | CTNND2, CXCR3, CYCS, Cystatin C, Cytochrome C, Cytokeratin 18, Cytokeratin 8, |
| | Cytokeratin basic, DBF4B/DRF1, DBI*, DCTN-50/DCTN2, DDAH1, DDAH1, DDX1, |
| | Destrin, DIP13B/appl2, DIP13B/appl2, DLG1, Dnpep, E-Cadherin, ECH1, ECHS1, |
| | ECHS1, EDIL3 (del-1), EDN-3, EDNRB/EDN3, EGFR, EIF4A3, ENTPD4, EpoR, EpoR, |
| | ERAB, ESD, ESD, ETS1, ETS1, ETS-2, EZH2, EZH2/KMT6, EZR, FABP5, FARSLA, |
| | FASN, FKBP5/FKBP51, FLNB, FTL (light and heavy), FUS, GAL3, gamma-catenin, |
| | GDF15, GDI2, GGPS1, GGPS1, GITRL, GloI, GLUD2, GM2A, GM-CSF, |
| | GOLM1/GOLPH2 Mab; clone 3B10, GOLPH2, GOLPH2, GPC6, GRP94, GSTP1, GSTP1, |
| | H3F3A, HADH/HADHSC, HGF, HIST1H3A, Histone H4, hK2/Kif2a, hnRNP A1, hnRNP |
| | A2B1, hnRNP C1 + C2, hnRNP K (F45)*, hnRNP L, hnRNP M1-M4, HOXB13, Hsp10/ |
| | HSPE1, Hsp40/DNAJB1, Hsp60, HSP90AA1, Hsp90B, HSPA1A, HSPB1, IDH2, IDH3B, |
| | IDH3B, IGFBP-2, IGFBP-3, IgG1, IgG2A, IgG2B, IL1alpha, IL1alpha, Integrin beta 7, |
| | IQGAP1, ITGAL, KLHL12/C3IP1, KLK1, KLK10, KLK11, KLK12, KLK13, KLK14, |
| | KLK15, KLK4, KLK5, KLK6, KLK7, KLK8, KLK9, Ku70 (XRCC6), Lamin B1, LAMP1, |
| | Lamp-2, LDH-A, LGALS3BP, LGALS8, Lipoamide Dehydrogenase, LLGL2, LSP1, LSP1, |
| | LTBP2, MATR3, MBD5, MDH2, MDM4, ME1, MKI67/Ki67, MMP 1, MMP 2, MMP 25, |
| | MMP10, MMP-14/MT1-MMP, MMP3, MMP7*, Mortalin, MTA1, nAnS, nAnS, |
| | Nav1.7/SCN9A, NCL, NDRG1, NKX3-1, NONO, Notch1, NOTCH4, NRP1/CD304, |
| | Nucleobindin-1, Nucleophosmin, p130/RBL2, p97/VCP, PAP - same as ACPP, PHGDH, |
| | PhIP, PIP3/BPNT1, PKA R2, PKM2, PKM2, PKP1, PKP3, PLEKHC1/Kindlin-2, PRDX2, |
| | PRKCSH, Prohibitin, Proteasome 19S 10B, Proteasome 20S beta 7, PSAP, PSMA, PSMA1, |
| | PSMA1, PSMD7, PSMD7, PSME3, PSP94/MSP/IGBF, PTBP1, PTEN, PTPN13/PTPL1, |
| | Rab1A, RAB3B, Rab5a, Rad51b, RPL10, RPL10, RPL14, RPL14, RPL19, RUVBL2, |
| | SCARB2, seprase/FAP, SerpinB6, SET, SH3PX1, SLC20A2, SLC3A2/CD98, SLC9A3R2, |
| | SMARCA4, Sorbitol Dehydrogenase, SPEN/RBM15, SPOCK1, SPR, SRVN, Stanniocalcin |
| | 2/STC2, STEAP1, Synaptogyrin 2/SYNGR2, Syndecan, SYNGR2, SYT9, TAF1B/ |
| | GRHL1, TBX5, TGFB, TGM2, TGN46/TGOLN2, TIMP-1, TLR3, TLR4 (CD284), TLR9/ |
| | CD289, TM9SF2, TMBIM6, TMPRSS1, TMPRSS2, TNFR1, TNFRI, TNFRII, TNFSF18/ |
| | GITRL, TNFα, TNFα, Tollip, TOM1, TOMM22, Trop2/TACSTD2, TSNAXIP1, TWEAK, |
| | U2AF2, uPA, uPAR/CD87, USP14, USP14, VAMP8, VASP, VDAC2, VEGFA, |
| | VEGFR1/FLT1, VEGFR2, VPS28, XRCC5/Ku80, XRCC5/Ku80 |
| Prostate Vesicles/ | EpCAM/TROP-1, HSA, Fibrinogen, GAPDH, Cholesterol Oxidase, MMP7, Complement |
| General Vesicles | Factor D/Adipsin, E-Cadherin, Transferrin Antibody, eNOS, IgM, CD9, Apolipoprotein B |
| | (Apo B), Ep-CAM, TBG, Kallekerin 3, IgA, IgG, Annexin V, IgG, Pyruvate Carboxylase, |
| | trypsin, AFP, TNF RI/TNFRSF1A, Aptamer CAR023, Aptamer CAR024, Aptamer CAR025, |
| | Aptamer CAR026 |
| Ribonucleoprotein | GW182, Ago2, miR-let-7a, miR-16, miR-22, miR-148a, miR-451, miR-92a, CD9, CD63, |
| complexes & | CD81 |
| vesicles | |
| Prostate Cancer | PCSA, Muc2, Adam10 |
| vesicles | |
| Prostate Cancer | Alkaline Phosphatase (AP), CD63, MyoD1, Neuron Specific Enolase, MAP1B, CNPase, |
| vesicles | Prohibitin, CD45RO, Heat Shock Protein 27, Collagen II, Laminin B1/b1, Gai1, CDw75, bcl- |
| | XL, Laminin-s, Ferritin, CD21, ADP-ribosylation Factor (ARF-6) |
| Prostate Cancer | CD56/NCAM-1, Heat Shock Protein 27/hsp27, CD45RO, MAP1B, MyoD1, |
| vesicles | CD45/T200/LCA, CD3zeta, Laminin-s, bcl-XL, Rad18, Gai1, Thymidylate Synthase, |
| | Alkaline Phosphatase (AP), CD63, MMP-16/MT3-MMP, Cyclin C, Neuron Specific |
| | Enolase, SIRP a1, Laminin B1/b1, Amyloid Beta (APP), SODD (Silencer of Death Domain), |
| | CDC37, Gab-1, E2F-2, CD6, Mast Cell Chymase, Gamma Glutamylcysteine Synthetase |
| | (GCS) |
| Prostate Cancer | EpCAM, MMP7, PCSA, BCNP, ADAM10, KLK2, SPDEF, CD81, MFGE8, IL-8 |
| vesicles | |
| Prostate Cancer | EpCAM, KLK2, PBP, SPDEF, SSX2, SSX4 |
| vesicles | |
| Prostate Cancer | ADAM-10, BCNP, CD9, EGFR, EpCam, IL1B, KLK2, MMP7, p53, PBP, PCSA, |
| vesicles | SERPINB3, SPDEF, SSX2, SSX4 |
| Androgen Receptor | GTF2F1, CTNNB1, PTEN, APPL1, GAPDH, CDC37, PNRC1, AES, UXT, RAN, PA2G4, |
| (AR) pathway | JUN, BAG1, UBE2I, HDAC1, COX5B, NCOR2, STUB1, HIPK3, PXN, NCOA4 |
| members in cMVs | |
| EGFR1 pathway | RALBP1, SH3BGRL, RBBP7, REPS1, SNRPD2, CEBPB, APPL1, MAP3K3, EEF1A1, |
| members in cMVs | GRB2, RAC1, SNCA, MAP2K3, CEBPA, CDC42, SH3KBP1, CBL, PTPN6, YWHAB, |
| | FOXO1, JAK1, KRT8, RALGDS, SMAD2, VAV1, NDUFA13, PRKCB1, MYC, JUN, |
| | RFXANK, HDAC1, HIST3H3, PEBP1, PXN, TNIP1, PKN2 |
| TNF-alpha | BCL3, SMARCE1, RPS11, CDC37, RPL6, RPL8, PAPOLA, PSMC1, CASP3, AKT2, |
| pathway members | MAP3K7IP2, POLR2L, TRADD, SMARCA4, HIST3H3, GNB2L1, PSMD1, PEBP1, |
| in cMVs | HSPB1, TNIP1, RPS13, ZFAND5, YWHAQ, COMMD1, COPS3, POLR1D, SMARCC2, |
| | MAP3K3, BIRC3, UBE2D2, HDAC2, CASP8, MCMI, PSMD7, YWHAG, NFKBIA, |
| | CAST, YWHAB, G3BP2, PSMD13, FBL, RELB, YWHAZ, SKP1, UBE2D3, PDCD2, |
| | HSP90AA1, HDAC1, KPNA2, RPL30, GTF2I, PFDN2 |
| Colorectal cancer | CD9, EGFR, NGAL, CD81, STEAP, CD24, A33, CD66E, EPHA2, Ferritin, GPR30, |
| | GPR110, MMP9, OPN, p53, TMEM211, TROP2, TGM2, TIMP, EGFR, DR3, UNC93A, |
| | MUC17, EpCAM, MUC1, MUC2, TSG101, CD63, B7H3 |
| Colorectal cancer | DR3, STEAP, epha2, TMEM211, unc93A, A33, CD24, NGAL, EpCam, MUC17, TROP2, |
| | TETS |
| Colorectal cancer | A33, AFP, ALIX, ALX4, ANCA, APC, ASCA, AURKA, AURKB, B7H3, BANK1, BCNP, |
| | BDNF, CA-19-9, CCSA-2, CCSA-3&4, CD10, CD24, CD44, CD63, CD66 CEA, CD66e |
| | CEA, CD81, CD9, CDA, C-Erb2, CRMP-2, CRP, CRTN, CXCL12, CYFRA21-1, DcR3, |
| | DLL4, DR3, EGFR, Epcam, EphA2, FASL, FRT, GAL3, GDF15, GPCR (GPR110), GPR30, |
| | GRO-1, HBD 1, HBD2, HNP1-3, IL-1B, IL8, IMP3, L1CAM, LAMN, MACC-1, |
| | MGC20553, MCP-1, M-CSF, MIC1, MIF, MMP7, MMP9, MS4A1, MUC1, MUC17, |
| | MUC2, Ncam, NGAL, NNMT, OPN, p53, PCSA, PDGFRB, PRL, PSMA, PSME3, Reg IV, |
| | SCRN1, Sept-9, SPARC, SPON2, SPR, SRVN, TFF3, TGM2, TIMP-1, TMEM211, TNF- |
| | alpha, TPA, TPS, Trail-R2, Trail-R4, TrKB, TROP2, Tsg 101, TWEAK, UNC93A, VEGFA |
| Colorectal cancer | miR 92, miR 21, miR 9, miR 491 |
| Colorectal cancer | miR-127-3p, miR-92a, miR-486-3p, miR-378 |
| Colorectal cancer | TMEM211, MUC1, CD24 and/or GPR110 (GPCR 110) |
| Colorectal cancer | hsa-miR-376c, hsa-miR-215, hsa-miR-652, hsa-miR-582-5p, hsa-miR-324-5p, hsa-miR- |
| | 1296, hsa-miR-28-5p, hsa-miR-190, hsa-miR-590-5p, hsa-miR-202, hsa-miR-195 |
| Colorectal cancer | A26C1A, A26C1B, A2M, ACAA2, ACE, ACOT7, ACP1, ACTA1, ACTA2, ACTB, |
| vesicle markers | ACTBL2, ACTBL3, ACTC1, ACTG1, ACTG2, ACTN1, ACTN2, ACTN4, ACTR3, |
| | ADAM10, ADSL, AGR2, AGR3, AGRN, AHCY, AHNAK, AKR1B10, ALB, ALDH16A1, |
| | ALDH1A1, ALDOA, ANXA1, ANXA11, ANXA2, ANXA2P2, ANXA4, ANXA5, ANXA6, |
| | AP2A1, AP2A2, APOA1, ARF1, ARF3, ARF4, ARF5, ARF6, ARHGDIA, ARPC3, |
| | ARPC5L, ARRDC1, ARVCF, ASCC3L1, ASNS, ATP1A1, ATP1A2, ATP1A3, ATP1B1, |
| | ATP4A, ATP5A1, ATP5B, ATP5I, ATP5L, ATP5O, ATP6AP2, B2M, BAIAP2, |
| | BAIAP2L1, BRI3BP, BSG, BUB3, C1orf58, C5orf32, CAD, CALM1, CALM2, CALM3, |
| | CAND1, CANX, CAPZA1, CBR1, CBR3, CCT2, CCT3, CCT4, CCT5, CCT6A, CCT7, |
| | CCT8, CD44, CD46, CD55, CD59, CD63, CD81, CD82, CD9, CDC42, CDH1, CDH17, |
| | CEACAM5, CFL1, CFL2, CHMP1A, CHMP2A, CHMP4B, CKB, CLDN3, CLDN4, |
| | CLDN7, CLIC1, CLIC4, CLSTN1, CLTC, CLTCL1, CLU, COL12A1, COPB1, COPB2, |
| | CORO1C, COX4I1, COX5B, CRYZ, CSPG4, CSRP1, CST3, CTNNA1, CTNNB1, |
| | CTNND1, CTTN, CYFIP1, DCD, DERA, DIP2A, DIP2B, DIP2C, DMBT1, DPEP1, DPP4, |
| | DYNC1H1, EDIL3, EEF1A1, EEF1A2, EEF1AL3, EEF1G, EEF2, EFNB1, EGFR, EHD1, |
| | EHD4, EIF3EIP, EIF3I, EIF4A1, EIF4A2, ENO1, ENO2, ENO3, EPHA2, EPHA5, EPHB1, |
| | EPHB2, EPHB3, EPHB4, EPPK1, ESD, EZR, F11R, F5, F7, FAM125A, FAM125B, |
| | FAM129B, FASLG, FASN, FAT, FCGBP, FER1L3, FKBP1A, FLNA, FLNB, FLOT1, |
| | FLOT2, G6PD, GAPDH, GARS, GCN1L1, GDI2, GK, GMDS, GNA13, GNAI2, GNAI3, |
| | GNAS, GNB1, GNB2, GNB2L1, GNB3, GNB4, GNG12, GOLGA7, GPA33, GPI, |
| | GPRC5A, GSN, GSTP1, H2AFJ, HADHA, hCG_1757335, HEPH, HIST1H2AB, |
| | HIST1H2AE, HIST1H2AJ, HIST1H2AK, HIST1H4A, HIST1H4B, HIST1H4C, HIST1H4D, |
| | HIST1H4E, HIST1H4F, HIST1H4H, HIST1H4I, HIST1H4J, HIST1H4K, HIST1H4L, |
| | HIST2H2AC, HIST2H4A, HIST2H4B, HIST3H2A, HIST4H4, HLA-A, HLA-A29.1, HLA- |
| | B, HLA-C, HLA-E, HLA-H, HNRNPA2B1, HNRNPH2, HPCAL1, HRAS, HSD17B4, |
| | HSP90AA1, HSP90AA2, HSP90AA4P, HSP90AB1, HSP90AB2P, HSP90AB3P, HSP90B1, |
| | HSPA1A, HSPA1B, HSPA1L, HSPA2, HSPA4, HSPA5, HSPA6, HSPA7, HSPA8, HSPA9, |
| | HSPD1, HSPE1, HSPG2, HYOU1, IDH1, IFITM1, IFITM2, IFITM3, IGH@, IGHG1, |
| | IGHG2, IGHG3, IGHG4, IGHM, IGHV4-31, IGK@, IGKC, IGKV1-5, IGKV2-24, IGKV3- |
| | 20, IGSF3, IGSF8, IQGAP1, IQGAP2, ITGA2, ITGA3, ITGA6, ITGAV, ITGB1, ITGB4, |
| | JUP, KIAA0174, KIAA1199, KPNB1, KRAS, KRT1, KRT10, KRT13, KRT14, KRT15, |
| | KRT16, KRT17, KRT18, KRT19, KRT2, KRT20, KRT24, KRT25, KRT27, KRT28, KRT3, |
| | KRT4, KRT5, KRT6A, KRT6B, KRT6C, KRT7, KRT75, KRT76, KRT77, KRT79, KRT8, |
| | KRT9, LAMA5, LAMP1, LDHA, LDHB, LFNG, LGALS3, LGALS3BP, LGALS4, LIMA1, |
| | LIN7A, LIN7C, LOC100128936, LOC100130553, LOC100133382, LOC100133739, |
| | LOC284889, LOC388524, LOC388720, LOC442497, LOC653269, LRP4, LRPPRC, |
| | LRSAM1, LSR, LYZ, MAN1A1, MAP4K4, MARCKS, MARCKSL1, METRNL, MFGE8, |
| | MICA, MIF, MINK1, MITD1, MMP7, MOBKL1A, MSN, MTCH2, MUC13, MYADM, |
| | MYH10, MYH11, MYH14, MYH9, MYL6, MYL6B, MYO1C, MYO1D, NARS, NCALD, |
| | NCSTN, NEDD4, NEDD4L, NME1, NME2, NOTCH1, NQO1, NRAS, P4HB, PCBP1, |
| | PCNA, PCSK9, PDCD6, PDCD6IP, PDIA3, PDXK, PEBP1, PFN1, PGK1, PHB, PHB2, |
| | PKM2, PLEC1, PLEKHB2, PLSCR3, PLXNA1, PLXNB2, PPIA, PPIB, PPP2R1A, PRDX1, |
| | PRDX2, PRDX3, PRDX5, PRDX6, PRKAR2A, PRKDC, PRSS23, PSMA2, PSMC6, |
| | PSMD11, PSMD3, PSME3, PTGFRN, PTPRF, PYGB, QPCT, QSOX1, RAB10, RAB11A, |
| | RAB11B, RAB13, RAB14, RAB15, RAB1A, RAB1B, RAB2A, RAB33B, RAB35, RAB43, |
| | RAB4B, RAB5A, RAB5B, RAB5C, RAB6A, RAB6B, RAB7A, RAB8A, RAB8B, RAC1, |
| | RAC3, RALA, RALB, RAN, RANP1, RAP1A, RAP1B, RAP2A, RAP2B, RAP2C, RDX, |
| | REG4, RHOA, RHOC, RHOG, ROCK2, RP11-631M21.2, RPL10A, RPL12, RPL6, RPL8, |
| | RPLP0, RPLP0-like, RPLP1, RPLP2, RPN1, RPS13, RPS14, RPS15A, RPS16, RPS18, |
| | RPS20, RPS21, RPS27A, RPS3, RPS4X, RPS4Y1, RPS4Y2, RPS7, RPS8, RPSA, |
| | RPSAP15, RRAS, RRAS2, RUVBL1, RUVBL2, S100A10, S100A11, S100A14, S100A16, |
| | S100A6, S100P, SDC1, SDC4, SDCBP, SDCBP2, SERINC1, SERINC5, SERPINA1, |
| | SERPINF1, SETD4, SFN, SLC12A2, SLC12A7, SLC16A1, SLC1A5, SLC25A4, SLC25A5, |
| | SLC25A6, SLC29A1, SLC2A1, SLC3A2, SLC44A1, SLC7A5, SLC9A3R1, SMPDL3B, |
| | SNAP23, SND1, SOD1, SORT1, SPTAN1, SPTBN1, SSBP1, SSR4, TACSTD1, TAGLN2, |
| | TBCA, TCEB1, TCP1, TF, TFRC, THBS1, TJP2, TKT, TMED2, TNFSF10, TNIK, |
| | TNKS1BP1, TNPO3, TOLLIP, TOMM22, TPI1, TPM1, TRAP1, TSG101, TSPAN1, |
| | TSPAN14, TSPAN15, TSPAN6, TSPAN8, TSTA3, TTYH3, TUBA1A, TUBA1B, |
| | TUBA1C, TUBA3C, TUBA3D, TUBA3E, TUBA4A, TUBA4B, TUBA8, TUBB, TUBB2A, |
| | TUBB2B, TUBB2C, TUBB3, TUBB4, TUBB4Q, TUBB6, TUFM, TXN, UBA1, UBA52, |
| | UBB, UBC, UBE2N, UBE2V2, UGDH, UQCRC2, VAMP1, VAMP3, VAMP8, VCP, VIL1, |
| | VPS25, VPS28, VPS35, VPS36, VPS37B, VPS37C, WDR1, YWHAB, YWHAE, YWHAG, |
| | YWHAH, YWHAQ, YWHAZ |
| Colorectal Cancer | hsa-miR-16, hsa-miR-25, hsa-miR-125b, hsa-miR-451, hsa-miR-200c, hsa-miR-140-3p, hsa- |
| | miR-658, hsa-miR-370, hsa-miR-1296, hsa-miR-636, hsa-miR-502-5p |
| Breast cancer | miR-21, miR-155, miR-206, miR-122a, miR-210, miR-21, miR-155, miR-206, miR-122a, |
| | miR-210, let-7, miR-10b, miR-125a, miR-125b, miR-145, miR-143, miR-145, miR-1b |
| Breast cancer | GAS5 |
| Breast cancer | ER, PR, HER2, MUC1, EGFR, KRAS, B-Raf, CYP2D6, hsp70, MART-1, TRP, HER2, |
| | hsp70, MART-1, TRP, HER2, ER, PR, Class III b-tubulin, VEGFA, ETV6-NTRK3, BCA- |
| | 225, hsp70, MART1, ER, VEGFA, Class III b-tubulin, HER2/neu (e.g., for Her2+ breast |
| | cancer), GPR30, ErbB4 (JM) isoform, MPR8, MISIIR, CD9, EphA2, EGFR, B7H3, PSM, |
| | PCSA, CD63, STEAP, CD81, ICAM1, A33, DR3, CD66e, MFG-E8, TROP-2, |
| | Mammaglobin, Hepsin, NPGP/NPFF2, PSCA, 5T4, NGAL, EpCam, neurokinin receptor-1 |
| | (NK-1 or NK-1R), NK-2, Pai-1, CD45, CD10, HER2/ERBB2, AGTR1, NPY1R, MUC1, |
| | ESA, CD133, GPR30, BCA225, CD24, CA15.3 (MUC1 secreted), CA27.29 (MUC1 |
| | secreted), NMDAR1, NMDAR2, MAGEA, CTAG1B, NY-ESO-1, SPB, SPC, NSE, PGP9.5, |
| | progesterone receptor (PR) or its isoform (PR(A) or PR(B)), P2RX7, NDUFB7, NSE, GAL3, |
| | osteopontin, CHI3L1, IC3b, mesothelin, SPA, AQP5, GPCR, hCEA-CAM, PTP IA-2, |
| | CABYR, TMEM211, ADAM28, UNC93A, MUC17, MUC2, IL10R-beta, BCMA, |
| | HVEM/TNFRSF14, Trappin-2, Elafin, ST2/IL1 R4, TNFRF14, CEACAM1, TPA1, LAMP, |
| | WF, WH1000, PECAM, BSA, TNFR |
| Breast cancer | CD9, MIS Rii, ER, CD63, MUC1, HER3, STAT3, VEGFA, BCA, CA125, CD24, EPCAM, |
| | ERB B4 |
| Breast cancer | CD10, NPGP/NPFF2, HER2/ERBB2, AGTR1, NPY1R, neurokinin receptor-1 (NK-1 or NK- |
| | 1R), NK-2, MUC1, ESA, CD133, GPR30, BCA225, CD24, CA15.3 (MUC1 secreted), |
| | CA27.29 (MUC1 secreted), NMDAR1, NMDAR2, MAGEA, CTAG1B, NY-ESO-1 |
| Breast cancer | SPB, SPC, NSE, PGP9.5, CD9, P2RX7, NDUFB7, NSE, GAL3, osteopontin, CHI3L1, |
| | EGFR, B7H3, IC3b, MUC1, mesothelin, SPA, PCSA, CD63, STEAP, AQP5, CD81, DR3, |
| | PSM, GPCR, EphA2, hCEA-CAM, PTP IA-2, CABYR, TMEM211, ADAM28, UNC93A, |
| | A33, CD24, CD10, NGAL, EpCam, MUC17, TROP-2, MUC2, IL10R-beta, BCMA, |
| | HVEM/TNFRSF14, Trappin-2 Elafin, ST2/IL1 R4, TNFRF14, CEACAM1, TPA1, LAMP, |
| | WF, WH1000, PECAM, BSA, TNFR |
| Breast cancer | BRCA, MUC-1, MUC 16, CD24, ErbB4, ErbB2 (HER2), ErbB3, HSP70, Mammaglobin, |
| | PR, PR(B), VEGFA |
| Breast cancer | CD9, HSP70, Gal3, MIS, EGFR, ER, ICB3, CD63, B7H4, MUC1, DLL4, CD81, ERB3, |
| | VEGF, BCA225, BRCA, CA125, CD174, CD24, ERB2, NGAL, GPR30, CYFRA21, CD31, |
| | cMET, MUC2, ERBB4 |
| Breast cancer | CD9, EphA2, EGFR, B7H3, PSMA, PCSA, CD63, STEAP, CD81, STEAP1, ICAM1 |
| | (CD54), PSMA, A33, DR3, CD66e, MFG-8e, TMEM211, TROP-2, EGFR, Mammoglobin, |
| | Hepsin, NPGP/NPFF2, PSCA, 5T4, NGAL, NK-2, EpCam, NK-1R, PSMA, 5T4, PAI-1, |
| | CD45 |
| Breast cancer | PGP9.5, CD9, HSP70, gal3-b2c10, EGFR, iC3b, PSMA, PCSA, CD63, MUC1, DLL4, |
| | CD81, B7-H3, HER 3 (ErbB3), MART-1, PSA, VEGF A, TIMP-1, GPCR GPR110, EphA2, |
| | MMP9, mmp7, TMEM211, UNC93a, BRCA, CA125 (MUC16), Mammaglobin, CD174 |
| | (Lewis y), CD66e CEA, CD24 c.sn3, C-erbB2, CD10, NGAL, epcam, CEA |
| | (carcinoembryonic Antigen), GPR30, CYFRA21-1, OPN, MUC17, hVEGFR2, MUC2, |
| | NCAM, ASPH, ErbB4, SPB, SPC, CD9, MS4A1, EphA2, MIS RII, HER2 (ErbB2), ER, PR |
| | (B), MRP8, CD63, B7H4, TGM2, CD81, DR3, STAT 3, MACC-1, TrKB, IL 6 Unc, OPG- |
| | 13, IL6R, EZH2, SCRN1, TWEAK, SERPINB3, CDAC1, BCA-225, DR3, A33, |
| | NPGP/NPFF2, TIMP1, BDNF, FRT, Ferritin heavy chain, seprase, p53, LDH, HSP, ost, p53, |
| | CXCL12, HAP, CRP, Gro-alpha, Tsg 101, GDF15 |
| Breast cancer | CD9, HSP70, Gal3, MIS (RII), EGFR, ER, ICB3, CD63, B7H4, MUC1, CD81, ERB3, |
| | MART1, STAT3, VEGF, BCA225, BRCA, CA125, CD174, CD24, ERB2, NGAL, GPR30, |
| | CYFRA21, CD31, cMET, MUC2, ERB4, TMEM211 |
| Breast Cancer | 5T4 (trophoblast), ADAM10, AGER/RAGE, APC, APP (β-amyloid), ASPH (A-10), B7H3 |
| | (CD276), BACE1, BAI3, BRCA1, BDNF, BIRC2, C1GALT1, CA125 (MUC16), |
| | Calmodulin 1, CCL2 (MCP-1), CD9, CD10, CD127 (IL7R), CD174, CD24, CD44, CD63, |
| | CD81, CEA, CRMP-2, CXCR3, CXCR4, CXCR6, CYFRA 21, derlin 1, DLL4, DPP6, E- |
| | CAD, EpCaM, EphA2 (H-77), ER(1) ESR1α, ER(2) ESR2β, Erb B4, Erbb2, erb3 (Erb-B3), |
| | PA2G4, FRT (FLT1), Gal3, GPR30 (G-coupled ER1), HAP1, HER3, HSP-27, HSP70, IC3b, |
| | IL8, insig, junction plakoglobin, Keratin 15, KRAS, Mammaglobin, MART1, MCT2, |
| | MFGE8, MMP9, MRP8, Muc1, MUC17, MUC2, NCAM, NG2 (CSPG4), Ngal, NHE-3, |
| | NT5E (CD73), ODC1, OPG, OPN, p53, PARK7, PCSA, PGP9.5 (PARK5), PR(B), PSA, |
| | PSMA, RAGE, STXBP4, Survivin, TFF3 (secreted), TIMP1, TIMP2, TMEM211, TRAF4 |
| | (scaffolding), TRAIL-R2 (death Receptor 5), TrkB, Tsg 101, UNC93a, VEGF A, VEGFR2, |
| | YB-1, VEGFR1, GCDPF-15 (PIP), BigH3 (TGFb1-induced protein), 5HT2B (serotonin |
| | receptor 2B), BRCA2, BACE 1, CDH1-cadherin |
| Breast Cancer | AK5.2, ATP6V1B1, CRABP1 |
| Breast Cancer | DST.3, GATA3, KRT81 |
| Breast Cancer | AK5.2, ATP6V1B1, CRABP1, DST.3, ELF5, GATA3, KRT81, LALBA, OXTR, RASL10A, |
| | SERHL, TFAP2A.1, TFAP2A.3, TFAP2C, VTCN1 |
| Breast Cancer | TRAP; Renal Cell Carcinoma; Filamin; 14.3.3, Pan; Prohibitin; c-fos; Ang-2; GSTmu; Ang- |
| | 1; FHIT; Rad51; Inhibin alpha; Cadherin-P; 14.3.3 gamma; p18INK4c; P504S; XRCC2; |
| | Caspase 5; CREB-Binding Protein; Estrogen Receptor; IL17; Claudin 2; Keratin 8; GAPDH; |
| | CD1; Keratin, LMW; Gamma Glutamylcysteine Synthetase(GCS)/Glutamate-cysteine |
| | Ligase; a-B-Crystallin; Pax-5; MMP-19; APC; IL-3; Keratin 8 (phospho-specific Ser73); |
| | TGF-beta 2; ITK; Oct-2/; DJ-1; B7-H2; Plasma Cell Marker; Rad18; Estriol; Chk1; Prolactin |
| | Receptor; Laminin Receptor; Histone H1; CD45RO; GnRH Receptor; IP10/CRG2; Actin, |
| | Muscle Specific; S100; Dystrophin; Tubulin-a; CD3zeta; CDC37; GABA a Receptor 1; |
| | MMP-7 (Matrilysin); Heregulin; Caspase 3; CD56/NCAM-1; Gastrin 1; SREBP-1 (Sterol |
| | Regulatory Element Binding Protein-1); MLH1; PGP9.5; Factor VIII Related Antigen; ADP- |
| | ribosylation Factor (ARF-6); MHC II (HLA-DR) Ia; Survivin; CD23; G-CSF; CD2; |
| | Calretinin; Neuron Specific Enolase; CD165; Calponin; CD95/Fas; Urocortin; Heat Shock |
| | Protein 27/hsp27; Topo II beta; Insulin Receptor; Keratin 5/8; sm; Actin, skeletal muscle; |
| | CA19-9; GluR1; GRIP1; CD79a mb-1; TdT; HRP; CD94; CCK-8; Thymidine |
| | Phosphorylase; CD57; Alkaline Phosphatase (AP); CD59/MACIF/MIRL/Protectin; |
| | GLUT-1; alpha-1-antitrypsin; Presenillin; Mucin 3 (MUC3); pS2; 14-3-3 beta; MMP-13 |
| | (Collagenase-3); Fli-1; mGluR5; Mast Cell Chymase; Laminin B1/b1; Neurofilament |
| | (160 kDa); CNPase; Amylin Peptide; Gail; CD6; alpha-1-antichymotrypsin; E2F-2; MyoD1 |
| Ductal carcinoma | Laminin B1/b1; E2F-2; TdT; Apolipoprotein D; Granulocyte; Alkaline Phosphatase (AP); |
| in situ (DCIS) | Heat Shock Protein 27/hsp27; CD95/Fas; pS2; Estriol; GLUT-1; Fibronectin; CD6; CCK-8; |
| | sm; Factor VIII Related Antigen; CD57; Plasminogen; CD71/Transferrin Receptor; Keratin |
| | 5/8; Thymidine Phosphorylase; CD45/T200/LCA; Epithelial Specific Antigen; Macrophage; |
| | CD10; MyoD1; Gail; bcl-XL; hPL; Caspase 3; Actin, skeletal muscle; IP10/CRG2; GnRH |
| | Receptor; p35nck5a; ADP-ribosylation Factor (ARF-6); Cdk4; alpha-1-antitrypsin; IL17; |
| | Neuron Specific Enolase; CD56/NCAM-1; Prolactin Receptor; Cdk7; CD79a mb-1; Collagen |
| | IV; CD94; Myeloid Specific Marker; Keratin 10; Pax-5; IgM (m-Heavy Chain); CD45RO; |
| | CA19-9; Mucin 2; Glucagon; Mast Cell Chymase; MLH1; CD1; CNPase; Parkin; MHC II |
| | (HLA-DR) Ia; B7-H2; Chk1; Lambda Light Chain; MHC II (HLA-DP and DR); Myogenin; |
| | MMP-7 (Matrilysin); Topo II beta; CD53; Keratin 19; Rad18; Ret Oncoprotein; MHC II |
| | (HLA-DP); E3-binding protein (ARM1); Progesterone Receptor; Keratin 8; IgG; IgA; |
| | Tubulin; Insulin Receptor Substrate-1; Keratin 15; DR3; IL-3; Keratin 10/13; Cyclin D3; |
| | MHC I (HLA25 and HLA-Aw32); Calmodulin; Neurofilament (160 kDa) |
| Ductal carcinoma | Macrophage; Fibronectin; Granulocyte; Keratin 19; Cyclin D3; CD45/T200/LCA; EGFR; |
| in situ (DCIS) v. | Thrombospondin; CD81/TAPA-1; Ruv C; Plasminogen; Collagen IV; Laminin B1/b1; CD10; |
| other Breast cancer | TdT; Filamin; bcl-XL; 14.3.3 gamma; 14.3.3, Pan; p170; Apolipoprotein D; CD71/ |
| | Transferrin Receptor; FHIT |
| Breast cancer | 5HT2B, 5T4 (trophoblast), ACO2, ACSL3, ACTN4, ADAM10, AGR2, AGR3, ALCAM, |
| microvesicles | ALDH6A1, ANGPTL4, ANO9, AP1G1, APC, APEX1, APLP2, APP (Amyloid precursor |
| | protein), ARCN1, ARHGAP35, ARL3, ASAH1, ASPH (A-10), ATP1B1, ATP1B3, ATP5I, |
| | ATP5O, ATXN1, B7H3, BACE1, BAI3, BAIAP2, BCA-200, BDNF, BigH3, BIRC2, |
| | BLVRB, BRCA, BST2, C1GALT1, C1GALT1C1, C20orf3, CA125, CACYBP, Calmodulin, |
| | CAPN1, CAPNS1, CCDC64B, CCL2 (MCP-1), CCT3, CD10(BD), CD127 (IL7R), CD174, |
| | CD24, CD44, CD80, CD86, CDH1, CDH5, CEA, CFL2, CHCHD3, CHMP3, CHRDL2, |
| | CIB1, CKAP4, COPA, COX5B, CRABP2, CRIP1, CRISPLDL CRMP-2, CRTAP, CTLA4, |
| | CUL3, CXCR3, CXCR4, CXCR6, CYB5B, CYB5R1, CYCS, CYFRA 21, DBI, DDX23, |
| | DDX39B, derlin 1, DHCR7, DHX9, DLD, DLL4, DNAJB1, DPP6, DSTN, eCadherin, |
| | EEF1D, EEF2, EFTUD2, EIF4A2, EIF4A3, EpCaM, EphA2, ER(1) (ESR1), ER(2) (ESR2), |
| | Erb B4, Erb2, erb3 (Erb-B3?), ERLIN2, ESD, FARSA, FASN, FEN1, FKBP5, FLNB, |
| | FOXP3, FUS, Gal3, GCDPF-15, GCNT2, GNA12, GNG5, GNPTG, GPC6, GPD2, GPER |
| | (GPR30), GSPT1, H3F3B, H3F3C, HADH, HAP1, HER3, HIST1H1C, HIST1H2AB, |
| | HIST1H3A, HIST1H3C, HIST1H3D, HIST1H3E, HIST1H3F, HIST1H3G, HIST1H3H, |
| | HIST1H3I, HIST1H3J, HIST2H2BF, HIST2H3A, HIST2H3C, HIST2H3D, HIST3H3, |
| | HMGB1, HNRNPA2B1, HNRNPAB, HNRNPC, HNRNPD, HNRNPH2, HNRNPK, |
| | HNRNPL, HNRNPM, HNRNPU, HPS3, HSP-27, HSP70, HSP90B1, HSPA1A, HSPA2, |
| | HSPA9, HSPE1, IC3b, IDE, IDH3B, IDO1, IFI30, IL1RL2, IL7, IL8, ILF2, ILF3, IQCG, |
| | ISOC2, IST1, ITGA7, ITGB7, junction plakoglobin, Keratin 15, KRAS, KRT19, KRT2, |
| | KRT7, KRT8, KRT9, KTN1, LAMP1, LMNA, LMNB1, LNPEP, LRPPRC, LRRC57, |
| | Mammaglobin, MAN1A1, MAN1A2, MART1, MATR3, MBD5, MCT2, MDH2, MFGE8, |
| | MFGE8, MGP, MMP9, MRP8, MUC1, MUC17, MUC2, MYO5B, MYOF, NAPA, NCAM, |
| | NCL, NG2 (CSPG4), Ngal, NHE-3, NME2, NONO, NPM1, NQO1, NT5E (CD73), ODC1, |
| | OPG, OPN (SC), OS9, p53, PACSIN3, PAICS, PARK7, PARVA, PC, PCNA, PCSA, PD-1, |
| | PD-L1, PD-L2, PGP9.5, PHB, PHB2, PIK3C2B, PKP3, PPL, PR(B), PRDX2, PRKCB, |
| | PRKCD, PRKDC, PSA, PSAP, PSMA, PSMB7, PSMD2, PSME3, PYCARD, RAB1A, |
| | RAB3D, RAB7A, RAGE, RBL2, RNPEP, RPL14, RPL27, RPL36, RPS25, RPS4X, |
| | RPS4Y1, RPS4Y2, RUVBL2, SET, SHMT2, SLAIN1, SLC39A14, SLC9A3R2, |
| | SMARCA4, SNRPD2, SNRPD3, SNX33, SNX9, SPEN, SPR, SQSTM1, SSBP1, ST3GAL1, |
| | STXBP4, SUB1, SUCLG2, Survivin, SYT9, TFF3 (secreted), TGOLN2, THBS1, TIMP1, |
| | TIMP2, TMED10, TMED4, TMED9, TMEM211, TOM1, TRAF4 (scaffolding), TRAIL-R2, |
| | TRAP1, TrkB, Tsg 101, TXNDC16, U2AF2, UEVLD, UFC1, UNC93a, USP14, VASP, |
| | VCP, VDAC1, VEGFA, VEGFR1, VEGFR2, VPS37C, WIZ, XRCC5, XRCC6, YB-1, |
| | YWHAZ |
| Lung cancer | Pgrmc1 (progesterone receptor membrane component 1)/sigma-2 receptor, STEAP, EZH2 |
| Lung cancer | Prohibitin, CD23, Amylin Peptide, HRP, Rad51, Pax-5, Oct-3/, GLUT-1, PSCA, |
| | Thrombospondin, FHIT, a-B-Crystallin, LewisA, Vacular Endothelial Growth |
| | Factor(VEGF), Hepatocyte Factor Homologue-4, Flt-4, GluR6/7, Prostate Apoptosis |
| | Response Protein-4, GluR1, Fli-1, Urocortin, S100A4, 14-3-3 beta, P504S, HDAC1, PGP9.5, |
| | DJ-1, COX2, MMP-19, Actin, skeletal muscle, Claudin 3, Cadherin-P, Collagen IX, |
| | p27Kip1, Cathepsin D, CD30 (Reed-Sternberg Cell Marker), Ubiquitin, FSH-b, TrxR2, |
| | CCK-8, Cyclin C, CD138, TGF-beta 2, Adrenocorticotrophic Hormone, PPAR-gamma, Bcl- |
| | 6, GLUT-3, IGF-I, mRANKL, Fas-ligand, Filamin, Calretinin, Oct-1, Parathyroid Hormone, |
| | Claudin 5, Claudin 4, Raf-1 (Phospho-specific), CDC14A Phosphatase, Mitochondria, APC, |
| | Gastrin 1, Ku (p80), Gai1, XPA, Maltose Binding Protein, Melanoma (gp100), |
| | Phosphotyrosine, Amyloid A, CXCR4/Fusin, Hepatic Nuclear Factor-3B, Caspase 1, HPV |
| | 16-E7, Axonal Growth Cones, Lck, Ornithine Decarboxylase, Gamma Glutamylcysteine |
| | Synthetase(GCS)/Glutamate-cysteine Ligase, ERCC1, Calmodulin, Caspase 7 (Mch 3), |
| | CD137 (4-1BB), Nitric Oxide Synthase, brain (bNOS), E2F-2, IL-10R, L-Plastin, CD18, |
| | Vimentin, CD50/ICAM-3, Superoxide Dismutase, Adenovirus Type 5 E1A, PHAS-I, |
| | Progesterone Receptor (phospho-specific) - Serine 294, MHC II (HLA-DQ), XPG, ER Ca+2 |
| | ATPase2, Laminin-s, E3-binding protein (ARM1), CD45RO, CD1, Cdk2, MMP-10 |
| | (Stromilysin-2), sm, Surfactant Protein B (Pro), Apolipoprotein D, CD46, Keratin 8 |
| | (phospho-specific Ser73), PCNA, PLAP, CD20, Syk, LH, Keratin 19, ADP-ribosylation |
| | Factor (ARF-6), Int-2 Oncoprotein, Luciferase, AIF (Apoptosis Inducing Factor), Grb2, bcl- |
| | X, CD16, Paxillin, MHC II (HLA-DP and DR), B-Cell, p21WAF1, MHC II (HLA-DR), |
| | Tyrosinase, E2F-1, Pds1, Calponin, Notch, CD26/DPP IV, SV40 Large T Antigen, Ku |
| | (p70/p80), Perforin, XPF, SIM Ag (SIMA-4D3), Cdk1/p34cdc2, Neuron Specific Enolase, b- |
| | 2-Microglobulin, DNA Polymerase Beta, Thyroid Hormone Receptor, Human, Alkaline |
| | Phosphatase (AP), Plasma Cell Marker, Heat Shock Protein 70/hsp70, TRP75/gp75, SRF |
| | (Serum Response Factor), Laminin B1/b1, Mast Cell Chymase, Caldesmon, CEA/CD66e, |
| | CD24, Retinoid X Receptor (hRXR), CD45/T200/LCA, Rabies Virus, Cytochrome c, DR3, |
| | bcl-XL, Fascin, CD71/Transferrin Receptor |
| Lung Cancer | miR-497 |
| Lung Cancer | Pgrmc1 |
| Ovarian Cancer | CA-125, CA 19-9, c-reactive protein, CD95(also called Fas, Fas antigen, Fas receptor, FasR, |
| | TNFRSF6, APT1 or APO-1), FAP-1, miR-200 microRNAs, EGFR, EGFRvIII, |
| | apolipoprotein AI, apolipoprotein CIII, myoglobin, tenascin C, MSH6, claudin-3, claudin-4, |
| | caveolin-1, coagulation factor III, CD9, CD36, CD37, CD53, CD63, CD81, CD136, CD147, |
| | Hsp70, Hsp90, Rab13, Desmocollin-1, EMP-2, CK7, CK20, GCDF15, CD82, Rab-5b, |
| | Annexin V, MFG-E8, HLA-DR. MiR-200 microRNAs (miR-200a, miR-200b, miR-200c), |
| | miR-141, miR-429, JNK, Jun |
| Prostate Cancer v | AQP2, BMP5, C16orf86, CXCL13, DST, ERCC1, GNAO1, KLHL5, MAP4K1, NELL2, |
| normal | PENK, PGF, POU3F1, PRSS21, SCML1, SEMG1, SMARCD3, SNAI2, TAF1C, TNNT3 |
| Prostate Cancer v | ADRB2, ARG2, C22orf32, CYorf14, EIF1AY, FEV, KLK2, KLK4, LRRC26, MAOA, |
| Breast Cancer | NLGN4Y, PNPLA7, PVRL3, SIM2, SLC30A4, SLC45A3, STX19, TRIM36, TRPM8 |
| Prostate Cancer v | ADRB2, BAIAP2L2, C19orf33, CDX1, CEACAM6, EEF1A2, ERN2, FAM110B, FOXA2, |
| Colorectal Cancer | KLK2, KLK4, LOC389816, LRRC26, MIPOL1, SLC45A3, SPDEF, TRIM31, TRIM36, |
| | ZNF613 |
| Prostate Cancer v | ASTN2, CAB39L, CRIP1, FAM110B, FEV, GSTP1, KLK2, KLK4, LOC389816, LRRC26, |
| Lung Cancer | MUC1, PNPLA7, SIM2, SLC45A3, SPDEF, TRIM36, TRPV6, ZNF613 |
| Prostate Cancer | miRs-26a + b, miR-15, miR-16, miR-195, miR-497, miR-424, miR-206, miR-342-5p, miR- |
| | 186, miR-1271, miR-600, miR-216b, miR-519 family, miR-203 |
| Integrins | ITGA1 (CD49a, VLA1), ITGA2 (CD49b, VLA2), ITGA3 (CD49c, VLA3), ITGA4 (CD49d, |
| | VLA4), ITGA5 (CD49e, VLA5), ITGA6 (CD49f, VLA6), ITGA7 (FLJ25220), ITGA8, |
| | ITGA9 (RLC), ITGA10, ITGA11 (HsT18964), ITGAD (CD11D, FLJ39841), ITGAE |
| | (CD103, HUMINAE), ITGAL (CD11a, LFA1A), ITGAM (CD11b, MAC-1), ITGAV |
| | (CD51, VNRA, MSK8), ITGAW, ITGAX (CD11c), ITGB1 (CD29, FNRB, MSK12, |
| | MDF20), ITGB2 (CD18, LFA-1, MAC-1, MFI7), ITGB3 (CD61, GP3A, GPIIIa), ITGB4 |
| | (CD104), ITGB5 (FLJ26658), ITGB6, ITGB7, ITGB8 |
| Glycoprotein | GpIa-IIa, GpIIb-IIIa, GpIIIb, GpIb, GpIX |
| Transcription | STAT3, EZH2, p53, MACC1, SPDEF, RUNX2, YB-1 |
| factors | |
| Kinases | AURKA, AURKB |
| Disease Markers | 6Ckine, Adiponectin, Adrenocorticotropic Hormone, Agouti-Related Protein, Aldose |
| | Reductase, Alpha-1-Antichymotrypsin, Alpha-1-Antitrypsin, Alpha-1-Microglobulin, Alpha- |
| | 2-Macroglobulin, Alpha-Fetoprotein, Amphiregulin, Angiogenin, Angiopoietin-2, |
| | Angiotensin-Converting Enzyme, Angiotensinogen, Annexin A1, Apolipoprotein A-I, |
| | Apolipoprotein A-II, Apolipoprotein A-IV, Apolipoprotein B, Apolipoprotein C-I, |
| | Apolipoprotein C-III, Apolipoprotein D, Apolipoprotein E, Apolipoprotein H, |
| | Apolipoprotein(a), AXL Receptor Tyrosine Kinase, B cell-activating Factor, B Lymphocyte |
| | Chemoattractant, Bcl-2-like protein 2, Beta-2-Microglobulin, Betacellulin, Bone |
| | Morphogenetic Protein 6, Brain-Derived Neurotrophic Factor, Calbindin, Calcitonin, Cancer |
| | Antigen 125, Cancer Antigen 15-3, Cancer Antigen 19-9, Cancer Antigen 72-4, |
| | Carcinoembryonic Antigen, Cathepsin D, CD 40 antigen, CD40 Ligand, CD5 Antigen-like, |
| | Cellular Fibronectin, Chemokine CC-4, Chromogranin-A, Ciliary Neurotrophic Factor, |
| | Clusterin, Collagen IV, Complement C3, Complement Factor H, Connective Tissue Growth |
| | Factor, Cortisol, C-Peptide, C-Reactive Protein, Creatine Kinase-MB, Cystatin-C, Endoglin, |
| | Endostatin, Endothelin-1, EN-RAGE, Eotaxin-1, Eotaxin-2, Eotaxin-3, Epidermal Growth |
| | Factor, Epiregulin, Epithelial cell adhesion molecule, Epithelial-Derived Neutrophil- |
| | Activating Protein 78, Erythropoietin, E-Selectin, Ezrin, Factor VII, Fas Ligand, FASLG |
| | Receptor, Fatty Acid-Binding Protein (adipocyte), Fatty Acid-Binding Protein (heart), Fatty |
| | Acid-Binding Protein (liver), Ferritin, Fetuin-A, Fibrinogen, Fibroblast Growth Factor 4, |
| | Fibroblast Growth Factor basic, Fibulin-1C, Follicle-Stimulating Hormone, Galectin-3, |
| | Gelsolin, Glucagon, Glucagon-like Peptide 1, Glucose-6-phosphate Isomerase, Glutamate- |
| | Cysteine Ligase Regulatory subunit, Glutathione S-Transferase alpha, Glutathione S- |
| | Transferase Mu 1, Granulocyte Colony-Stimulating Factor, Granulocyte-Macrophage |
| | Colony-Stimulating Factor, Growth Hormone, Growth-Regulated alpha protein, Haptoglobin, |
| | HE4, Heat Shock Protein 60, Heparin-Binding EGF-Like Growth Factor, Hepatocyte Growth |
| | Factor, Hepatocyte Growth Factor Receptor, Hepsin, Human Chorionic Gonadotropin beta, |
| | Human Epidermal Growth Factor Receptor 2, Immunoglobulin A, Immunoglobulin E, |
| | Immunoglobulin M, Insulin, Insulin-like Growth Factor I, Insulin-like Growth Factor- |
| | Binding Protein 1, Insulin-like Growth Factor-Binding Protein 2, Insulin-like Growth Factor- |
| | Binding Protein 3, Insulin-like Growth Factor Binding Protein 4, Insulin-like Growth Factor |
| | Binding Protein 5, Insulin-like Growth Factor Binding Protein 6, Intercellular Adhesion |
| | Molecule 1, Interferon gamma, Interferon gamma Induced Protein 10, Interferon-inducible T- |
| | cell alpha chemoattractant, Interleukin-1 alpha, Interleukin-1 beta, Interleukin-1 Receptor |
| | antagonist, Interleukin-2, Interleukin-2 Receptor alpha, Interleukin-3, Interleukin-4, |
| | Interleukin-5, Interleukin-6, Interleukin-6 Receptor, Interleukin-6 Receptor subunit beta, |
| | Interleukin-7, Interleukin-8, Interleukin-10, Interleukin-11, Interleukin-12 Subunit p40, |
| | Interleukin-12 Subunit p70, Interleukin-13, Interleukin-15, Interleukin-16, Interleukin-25, |
| | Kallikrein 5, Kallikrein-7, Kidney Injury Molecule-1, Lactoylglutathione lyase, Latency- |
| | Associated Peptide of Transforming Growth Factor beta 1, Lectin-Like Oxidized LDL |
| | Receptor 1, Leptin, Luteinizing Hormone, Lymphotactin, Macrophage Colony-Stimulating |
| | Factor 1, Macrophage Inflammatory Protein-1 alpha, Macrophage Inflammatory Protein-1 |
| | beta, Macrophage Inflammatory Protein-3 alpha, Macrophage inflammatory protein 3 beta, |
| | Macrophage Migration Inhibitory Factor, Macrophage-Derived Chemokine, Macrophage- |
| | Stimulating Protein, Malondialdehyde-Modified Low-Density Lipoprotein, Maspin, Matrix |
| | Metalloproteinase-1, Matrix Metalloproteinase-2, Matrix Metalloproteinase-3, Matrix |
| | Metalloproteinase-7, Matrix Metalloproteinase-9, Matrix Metalloproteinase-9, Matrix |
| | Metalloproteinase-10, Mesothelin, MHC class I chain-related protein A, Monocyte |
| | Chemotactic Protein 1, Monocyte Chemotactic Protein 2, Monocyte Chemotactic Protein 3, |
| | Monocyte Chemotactic Protein 4, Monokine Induced by Gamma Interferon, Myeloid |
| | Progenitor Inhibitory Factor 1, Myeloperoxidase, Myoglobin, Nerve Growth Factor beta, |
| | Neuronal Cell Adhesion Molecule, Neuron-Specific Enolase, Neuropilin-1, Neutrophil |
| | Gelatinase-Associated Lipocalin, NT-proBNP, Nucleoside diphosphate kinase B, |
| | Osteopontin, Osteoprotegerin, Pancreatic Polypeptide, Pepsinogen I, Peptide YY, |
| | Peroxiredoxin-4, Phosphoserine Aminotransferase, Placenta Growth Factor, Plasminogen |
| | Activator Inhibitor 1, Platelet-Derived Growth Factor BB, Pregnancy-Associated Plasma |
| | Protein A, Progesterone, Proinsulin (inc. Total or Intact), Prolactin, Prostasin, Prostate- |
| | Specific Antigen (inc. Free PSA), Prostatic Acid Phosphatase, Protein S100-A4, Protein |
| | S100-A6, Pulmonary and Activation-Regulated Chemokine, Receptor for advanced |
| | glycosylation end products, Receptor tyrosine-protein kinase erbB-3, Resistin, S100 calcium- |
| | binding protein B, Secretin, Serotransferrin, Serum Amyloid P-Component, Serum Glutamic |
| | Oxaloacetic Transaminase, Sex Hormone-Binding Globulin, Sortilin, Squamous Cell |
| | Carcinoma Antigen-1, Stem Cell Factor, Stromal cell-derived Factor-1, Superoxide |
| | Dismutase 1 (soluble), T Lymphocyte-Secreted Protein I-309, Tamm-Horsfall Urinary |
| | Glycoprotein, T-Cell-Specific Protein RANTES, Tenascin-C, Testosterone, Tetranectin, |
| | Thrombomodulin, Thrombopoietin, Thrombospondin-1, Thyroglobulin, Thyroid-Stimulating |
| | Hormone, Thyroxine-Binding Globulin, Tissue Factor, Tissue Inhibitor of Metalloproteinases |
| | 1, Tissue type Plasminogen activator, TNF-Related Apoptosis-Inducing Ligand Receptor 3, |
| | Transforming Growth Factor alpha, Transforming Growth Factor beta-3, Transthyretin, |
| | Trefoil Factor 3, Tumor Necrosis Factor alpha, Tumor Necrosis Factor beta, Tumor Necrosis |
| | Factor Receptor I, Tumor necrosis Factor Receptor 2, Tyrosine kinase with Ig and EGF |
| | homology domains 2, Urokinase-type Plasminogen Activator, Urokinase-type plasminogen |
| | activator Receptor, Vascular Cell Adhesion Molecule-1, Vascular Endothelial Growth Factor, |
| | Vascular endothelial growth Factor B, Vascular Endothelial Growth Factor C, Vascular |
| | endothelial growth Factor D, Vascular Endothelial Growth Factor Receptor 1, Vascular |
| | Endothelial Growth Factor Receptor 2, Vascular endothelial growth Factor Receptor 3, |
| | Vitamin K-Dependent Protein S, Vitronectin, von Willebrand Factor, YKL-40 |
| Disease Markers | Adiponectin, Adrenocorticotropic Hormone, Agouti-Related Protein, Alpha-1- |
| | Antichymotrypsin, Alpha-1-Antitrypsin, Alpha-1-Microglobulin, Alpha-2-Macroglobulin, |
| | Alpha-Fetoprotein, Amphiregulin, Angiopoietin-2, Angiotensin-Converting Enzyme, |
| | Angiotensinogen, Apolipoprotein A-I, Apolipoprotein A-II, Apolipoprotein A-IV, |
| | Apolipoprotein B, Apolipoprotein C-I, Apolipoprotein C-III, Apolipoprotein D, |
| | Apolipoprotein E, Apolipoprotein H, Apolipoprotein(a), AXL Receptor Tyrosine Kinase, B |
| | Lymphocyte Chemoattractant, Beta-2-Microglobulin, Betacellulin, Bone Morphogenetic |
| | Protein 6, Brain-Derived Neurotrophic Factor, Calbindin, Calcitonin, Cancer Antigen 125, |
| | Cancer Antigen 19-9, Carcinoembryonic Antigen, CD 40 antigen, CD40 Ligand, CD5 |
| | Antigen-like, Chemokine CC-4, Chromogranin-A, Ciliary Neurotrophic Factor, Clusterin, |
| | Complement C3, Complement Factor H, Connective Tissue Growth Factor, Cortisol, C- |
| | Peptide, C-Reactive Protein, Creatine Kinase-MB, Cystatin-C, Endothelin-1, EN-RAGE, |
| | Eotaxin-1, Eotaxin-3, Epidermal Growth Factor, Epiregulin, Epithelial-Derived Neutrophil- |
| | Activating Protein 78, Erythropoietin, E-Selectin, Factor VII, Fas Ligand, FASLG Receptor, |
| | Fatty Acid-Binding Protein (heart), Ferritin, Fetuin-A, Fibrinogen, Fibroblast Growth Factor |
| | 4, Fibroblast Growth Factor basic, Follicle-Stimulating Hormone, Glucagon, Glucagon-like |
| | Peptide 1, Glutathione S-Transferase alpha, Granulocyte Colony-Stimulating Factor, |
| | Granulocyte-Macrophage Colony-Stimulating Factor, Growth Hormone, Growth-Regulated |
| | alpha protein, Haptoglobin, Heat Shock Protein 60, Heparin-Binding EGF-Like Growth |
| | Factor, Hepatocyte Growth Factor, Immunoglobulin A, Immunoglobulin E, Immunoglobulin |
| | M, Insulin, Insulin-like Growth Factor I, Insulin-like Growth Factor-Binding Protein 2, |
| | Intercellular Adhesion Molecule 1, Interferon gamma, Interferon gamma Induced Protein 10, |
| | Interleukin-1 alpha, Interleukin-1 beta, Interleukin-1 Receptor antagonist, Interleukin-2, |
| | Interleukin-3, Interleukin-4, Interleukin-5, Interleukin-6, Interleukin-6 Receptor, Interleukin- |
| | 7, Interleukin-8, Interleukin-10, Interleukin-11, Interleukin-12 Subunit p40, Interleukin-12 |
| | Subunit p70, Interleukin-13, Interleukin-15, Interleukin-16, Interleukin-25, Kidney Injury |
| | Molecule-1, Lectin-Like Oxidized LDL Receptor 1, Leptin, Luteinizing Hormone, |
| | Lymphotactin, Macrophage Colony-Stimulating Factor 1, Macrophage Inflammatory Protein- |
| | 1 alpha, Macrophage Inflammatory Protein-1 beta, Macrophage Inflammatory Protein-3 |
| | alpha, Macrophage Migration Inhibitory Factor, Macrophage-Derived Chemokine, |
| | Malondialdehyde-Modified Low-Density Lipoprotein, Matrix Metalloproteinase-1, Matrix |
| | Metalloproteinase-2, Matrix Metalloproteinase-3, Matrix Metalloproteinase-7, Matrix |
| | Metalloproteinase-9, Matrix Metalloproteinase-9, Matrix Metalloproteinase-10, Monocyte |
| | Chemotactic Protein 1, Monocyte Chemotactic Protein 2, Monocyte Chemotactic Protein 3, |
| | Monocyte Chemotactic Protein 4, Monokine Induced by Gamma Interferon, Myeloid |
| | Progenitor Inhibitory Factor 1, Myeloperoxidase, Myoglobin, Nerve Growth Factor beta, |
| | Neuronal Cell Adhesion Molecule, Neutrophil Gelatinase-Associated Lipocalin, NT-proBNP, |
| | Osteopontin, Pancreatic Polypeptide, Peptide YY, Placenta Growth Factor, Plasminogen |
| | Activator Inhibitor 1, Platelet-Derived Growth Factor BB, Pregnancy-Associated Plasma |
| | Protein A, Progesterone, Proinsulin (inc. Intact or Total), Prolactin, Prostate-Specific Antigen |
| | (inc. Free PSA), Prostatic Acid Phosphatase, Pulmonary and Activation-Regulated |
| | Chemokine, Receptor for advanced glycosylation end products, Resistin, S100 calcium- |
| | binding protein B, Secretin, Serotransferrin, Serum Amyloid P-Component, Serum Glutamic |
| | Oxaloacetic Transaminase, Sex Hormone-Binding Globulin, Sortilin, Stem Cell Factor, |
| | Superoxide Dismutase 1 (soluble), T Lymphocyte-Secreted Protein I-309, Tamm-Horsfall |
| | Urinary Glycoprotein, T-Cell-Specific Protein RANTES, Tenascin-C, Testosterone, |
| | Thrombomodulin, Thrombopoietin, Thrombospondin-1, Thyroid-Stimulating Hormone, |
| | Thyroxine-Binding Globulin, Tissue Factor, Tissue Inhibitor of Metalloproteinases 1, TNF- |
| | Related Apoptosis-Inducing Ligand Receptor 3, Transforming Growth Factor alpha, |
| | Transforming Growth Factor beta-3, Transthyretin, Trefoil Factor 3, Tumor Necrosis Factor |
| | alpha, Tumor Necrosis Factor beta, Tumor necrosis Factor Receptor 2, Vascular Cell |
| | Adhesion Molecule-1, Vascular Endothelial Growth Factor, Vitamin K-Dependent Protein S, |
| | Vitronectin, von Willebrand Factor |
| Oncology | 6Ckine, Aldose Reductase, Alpha-Fetoprotein, Amphiregulin, Angiogenin, Annexin A1, B |
| | cell-activating Factor, B Lymphocyte Chemoattractant, Bcl-2-like protein 2, Betacellulin, |
| | Cancer Antigen 125, Cancer Antigen 15-3, Cancer Antigen 19-9, Cancer Antigen 72-4, |
| | Carcinoembryonic Antigen, Cathepsin D, Cellular Fibronectin, Collagen IV, Endoglin, |
| | Endostatin, Eotaxin-2, Epidermal Growth Factor, Epiregulin, Epithelial cell adhesion |
| | molecule, Ezrin, Fatty Acid-Binding Protein (adipocyte), Fatty Acid-Binding Protein (liver), |
| | Fibroblast Growth Factor basic, Fibulin-1C, Galectin-3, Gelsolin, Glucose-6-phosphate |
| | Isomerase, Glutamate-Cysteine Ligase Regulatory subunit, Glutathione S-Transferase Mu 1, |
| | HE4, Heparin-Binding EGF-Like Growth Factor, Hepatocyte Growth Factor, Hepatocyte |
| | Growth Factor Receptor, Hepsin, Human Chorionic Gonadotropin beta, Human Epidermal |
| | Growth Factor Receptor 2, Insulin-like Growth Factor-Binding Protein 1, Insulin-like Growth |
| | Factor-Binding Protein 2, Insulin-like Growth Factor-Binding Protein 3, Insulin-like Growth |
| | Factor Binding Protein 4, Insulin-like Growth Factor Binding Protein 5, Insulin-like Growth |
| | Factor Binding Protein 6, Interferon gamma Induced Protein 10, Interferon-inducible T-cell |
| | alpha chemoattractant, Interleukin-2 Receptor alpha, Interleukin-6, Interleukin-6 Receptor |
| | subunit beta, Kallikrein 5, Kallikrein-7, Lactoylglutathione lyase, Latency-Associated Peptide |
| | of Transforming Growth Factor beta 1, Leptin, Macrophage inflammatory protein 3 beta, |
| | Macrophage Migration Inhibitory Factor, Macrophage-Stimulating Protein, Maspin, Matrix |
| | Metalloproteinase-2, Mesothelin, MHC class I chain-related protein A, Monocyte |
| | Chemotactic Protein 1, Monokine Induced by Gamma Interferon, Neuron-Specific Enolase, |
| | Neuropilin-1, Neutrophil Gelatinase-Associated Lipocalin, Nucleoside diphosphate kinase B, |
| | Osteopontin, Osteoprotegerin, Pepsinogen I, Peroxiredoxin-4, Phosphoserine |
| | Aminotransferase, Placenta Growth Factor, Platelet-Derived Growth Factor BB, Prostasin, |
| | Protein S100-A4, Protein S100-A6, Receptor tyrosine-protein kinase erbB-3, Squamous Cell |
| | Carcinoma Antigen-1, Stromal cell-derived Factor-1, Tenascin-C, Tetranectin, |
| | Thyroglobulin, Tissue type Plasminogen activator, Transforming Growth Factor alpha, |
| | Tumor Necrosis Factor Receptor I, Tyrosine kinase with Ig and EGF homology domains 2, |
| | Urokinase-type Plasminogen Activator, Urokinase-type plasminogen activator Receptor, |
| | Vascular Endothelial Growth Factor, Vascular endothelial growth Factor B, Vascular |
| | Endothelial Growth Factor C, Vascular endothelial growth Factor D, Vascular Endothelial |
| | Growth Factor Receptor 1, Vascular Endothelial Growth Factor Receptor 2, Vascular |
| | endothelial growth Factor Receptor 3, YKL-40 |
| Disease | Adiponectin, Alpha-1-Antitrypsin, Alpha-2-Macroglobulin, Alpha-Fetoprotein, |
| | Apolipoprotein A-I, Apolipoprotein C-III, Apolipoprotein H, Apolipoprotein(a), Beta-2- |
| | Microglobulin, Brain-Derived Neurotrophic Factor, Calcitonin, Cancer Antigen 125, Cancer |
| | Antigen 19-9, Carcinoembryonic Antigen, CD 40 antigen, CD40 Ligand, Complement C3, C- |
| | Reactive Protein, Creatine Kinase-MB, Endothelin-1, EN-RAGE, Eotaxin-1, Epidermal |
| | Growth Factor, Epithelial-Derived Neutrophil-Activating Protein 78, Erythropoietin, Factor |
| | VII, Fatty Acid-Binding Protein (heart), Ferritin, Fibrinogen, Fibroblast Growth Factor basic, |
| | Granulocyte Colony-Stimulating Factor, Granulocyte-Macrophage Colony-Stimulating |
| | Factor, Growth Hormone, Haptoglobin, Immunoglobulin A, Immunoglobulin E, |
| | Immunoglobulin M, Insulin, Insulin-like Growth Factor I, Intercellular Adhesion Molecule 1, |
| | Interferon gamma, Interleukin-1 alpha, Interleukin-1 beta, Interleukin-1 Receptor antagonist, |
| | Interleukin-2, Interleukin-3, Interleukin-4, Interleukin-5, Interleukin-6, Interleukin-7, |
| | Interleukin-8, Interleukin-10, Interleukin-12 Subunit p40, Interleukin-12 Subunit p70, |
| | Interleukin-13, Interleukin-15, Interleukin-16, Leptin, Lymphotactin, Macrophage |
| | Inflammatory Protein-1 alpha, Macrophage Inflammatory Protein-1 beta, Macrophage- |
| | Derived Chemokine, Matrix Metalloproteinase-2, Matrix Metalloproteinase-3, Matrix |
| | Metalloproteinase-9, Monocyte Chemotactic Protein 1, Myeloperoxidase, Myoglobin, |
| | Plasminogen Activator Inhibitor 1, Pregnancy-Associated Plasma Protein A, Prostate- |
| | Specific Antigen (inc. Free PSA), Prostatic Acid Phosphatase, Serum Amyloid P-Component, |
| | Serum Glutamic Oxaloacetic Transaminase, Sex Hormone-Binding Globulin, Stem Cell |
| | Factor, T-Cell-Specific Protein RANTES, Thrombopoietin, Thyroid-Stimulating Hormone, |
| | Thyroxine-Binding Globulin, Tissue Factor, Tissue Inhibitor of Metalloproteinases 1, Tumor |
| | Necrosis Factor alpha, Tumor Necrosis Factor beta, Tumor Necrosis Factor Receptor 2, |
| | Vascular Cell Adhesion Molecule-1, Vascular Endothelial Growth Factor, von Willebrand |
| | Factor |
| Neurological | Alpha-1-Antitrypsin, Apolipoprotein A-I, Apolipoprotein A-II, Apolipoprotein B, |
| | Apolipoprotein C-I, Apolipoprotein H, Beta-2-Microglobulin, Betacellulin, Brain-Derived |
| | Neurotrophic Factor, Calbindin, Cancer Antigen 125, Carcinoembryonic Antigen, CD5 |
| | Antigen-like, Complement C3, Connective Tissue Growth Factor, Cortisol, Endothelin-1, |
| | Epidermal Growth Factor Receptor, Ferritin, Fetuin-A, Follicle-Stimulating Hormone, |
| | Haptoglobin, Immunoglobulin A, Immunoglobulin M, Intercellular Adhesion Molecule 1, |
| | Interleukin-6 Receptor, Interleukin-7, Interleukin-10, Interleukin-11, Interleukin-17, Kidney |
| | Injury Molecule-1, Luteinizing Hormone, Macrophage-Derived Chemokine, Macrophage |
| | Migration Inhibitory Factor, Macrophage Inflammatory Protein-1 alpha, Matrix |
| | Metalloproteinase-2, Monocyte Chemotactic Protein 2, Peptide YY, Prolactin, Prostatic Acid |
| | Phosphatase, Serotransferrin, Serum Amyloid P-Component, Sortilin, Testosterone, |
| | Thrombopoietin, Thyroid-Stimulating Hormone, Tissue Inhibitor of Metalloproteinases 1, |
| | TNF-Related Apoptosis-Inducing Ligand Receptor 3, Tumor necrosis Factor Receptor 2, |
| | Vascular Endothelial Growth Factor, Vitronectin |
| Cardiovascular | Adiponectin, Apolipoprotein A-I, Apolipoprotein B, Apolipoprotein C-III, Apolipoprotein D, |
| | Apolipoprotein E, Apolipoprotein H, Apolipoprotein(a), Clusterin, C-Reactive Protein, |
| | Cystatin-C, EN-RAGE, E-Selectin, Fatty Acid-Binding Protein (heart), Ferritin, Fibrinogen, |
| | Haptoglobin, Immunoglobulin M, Intercellular Adhesion Molecule 1, Interleukin-6, |
| | Interleukin-8, Lectin-Like Oxidized LDL Receptor 1, Leptin, Macrophage Inflammatory |
| | Protein-1 alpha, Macrophage Inflammatory Protein-1 beta, Malondialdehyde-Modified Low- |
| | Density Lipoprotein, Matrix Metalloproteinase-1, Matrix Metalloproteinase-10, Matrix |
| | Metalloproteinase-2, Matrix Metalloproteinase-3, Matrix Metalloproteinase-7, Matrix |
| | Metalloproteinase-9, Monocyte Chemotactic Protein 1, Myeloperoxidase, Myoglobin, NT- |
| | proBNP, Osteopontin, Plasminogen Activator Inhibitor 1, P-Selectin, Receptor for advanced |
| | glycosylation end products, Serum Amyloid P-Component, Sex Hormone-Binding Globulin, |
| | T-Cell-Specific Protein RANTES, Thrombomodulin, Thyroxine-Binding Globulin, Tissue |
| | Inhibitor of Metalloproteinases 1, Tumor Necrosis Factor alpha, Tumor necrosis Factor |
| | Receptor 2, Vascular Cell Adhesion Molecule-1, von Willebrand Factor |
| Inflammatory | Alpha-1-Antitrypsin, Alpha-2-Macroglobulin, Beta-2-Microglobulin, Brain-Derived |
| | Neurotrophic Factor, Complement C3, C-Reactive Protein, Eotaxin-1, Factor VII, Ferritin, |
| | Fibrinogen, Granulocyte-Macrophage Colony-Stimulating Factor, Haptoglobin, Intercellular |
| | Adhesion Molecule 1, Interferon gamma, Interleukin-1 alpha, Interleukin-1 beta, Interleukin- |
| | 1 Receptor antagonist, Interleukin-2, Interleukin-3, Interleukin-4, Interleukin-5, Interleukin-6, |
| | Interleukin-7, Interleukin-8, Interleukin-10, Interleukin-12 Subunit p40, Interleukin-12 |
| | Subunit p70, Interleukin-15, Interleukin-17, Interleukin-23, Macrophage Inflammatory |
| | Protein-1 alpha, Macrophage Inflammatory Protein-1 beta, Matrix Metalloproteinase-2, |
| | Matrix Metalloproteinase-3, Matrix Metalloproteinase-9, Monocyte Chemotactic Protein 1, |
| | Stem Cell Factor, T-Cell-Specific Protein RANTES, Tissue Inhibitor of Metalloproteinases 1, |
| | Tumor Necrosis Factor alpha, Tumor Necrosis Factor beta, Tumor necrosis Factor Receptor |
| | 2, Vascular Cell Adhesion Molecule-1, Vascular Endothelial Growth Factor, Vitamin D- |
| | Binding Protein, von Willebrand Factor |
| Metabolic | Adiponectin, Adrenocorticotropic Hormone, Angiotensin-Converting Enzyme, |
| | Angiotensinogen, Complement C3 alpha des arg, Cortisol, Follicle-Stimulating Hormone, |
| | Galanin, Glucagon, Glucagon-like Peptide 1, Insulin, Insulin-like Growth Factor I, Leptin, |
| | Luteinizing Hormone, Pancreatic Polypeptide, Peptide YY, Progesterone, Prolactin, Resistin, |
| | Secretin, Testosterone |
| Kidney | Alpha-1-Microglobulin, Beta-2-Microglobulin, Calbindin, Clusterin, Connective Tissue |
| | Growth Factor, Creatinine, Cystatin-C, Glutathione S-Transferase alpha, Kidney Injury |
| | Molecule-1, Microalbumin, Neutrophil Gelatinase-Associated Lipocalin, Osteopontin, |
| | Tamm-Horsfall Urinary Glycoprotein, Tissue Inhibitor of Metalloproteinases 1, Trefoil |
| | Factor 3, Vascular Endothelial Growth Factor |
| Cytokines | Granulocyte-Macrophage Colony-Stimulating Factor, Interferon gamma, Interleukin-2, |
| | Interleukin-3, Interleukin-4, Interleukin-5, Interleukin-6, Interleukin-7, Interleukin-8, |
| | Interleukin-10, Macrophage Inflammatory Protein-1 alpha, Macrophage Inflammatory |
| | Protein-1 beta, Matrix Metalloproteinase-2, Monocyte Chemotactic Protein 1, Tumor |
| | Necrosis Factor alpha, Tumor Necrosis Factor beta, Brain-Derived Neurotrophic Factor, |
| | Eotaxin-1, Intercellular Adhesion Molecule 1, Interleukin-1 alpha, Interleukin-1 beta, |
| | Interleukin-1 Receptor antagonist, Interleukin-12 Subunit p40, Interleukin-12 Subunit p70, |
| | Interleukin-15, Interleukin-17, Interleukin-23, Matrix Metalloproteinase-3, Stem Cell Factor, |
| | Vascular Endothelial Growth Factor |
| Protein | 14.3.3 gamma, 14.3.3 (Pan), 14-3-3 beta, 6-Histidine, a-B-Crystallin, Acinus, Actin beta, |
| | Actin (Muscle Specific), Actin (Pan), Actin (skeletal muscle), Activin Receptor Type II, |
| | Adenovirus, Adenovirus Fiber, Adenovirus Type 2 E1A, Adenovirus Type 5 E1A, ADP- |
| | ribosylation Factor (ARF-6), Adrenocorticotrophic Hormone, AIF (Apoptosis Inducing |
| | Factor), Alkaline Phosphatase (AP), Alpha Fetoprotein (AFP), Alpha Lactalbumin, alpha-1- |
| | antichymotrypsin, alpha-1-antitrypsin, Amphiregulin, Amylin Peptide, Amyloid A, Amyloid |
| | A4 Protein Precursor, Amyloid Beta (APP), Androgen Receptor, Ang-1, Ang-2, APC, |
| | APC11, APC2, Apolipoprotein D, A-Raf, ARC, Ask1/MAPKKK5, ATM, Axonal Growth |
| | Cones, b Galactosidase, b-2-Microglobulin, B7-H2, BAG-1, Bak, Bax, B-Cell, B-cell Linker |
| | Protein (BLNK), Bcl10/CIPER/CLAP/mE10, bcl-2a, Bcl-6, bcl-X, bcl-XL, Bim (BOD), |
| | Biotin, Bonzo/STRL33/TYMSTR, Bovine Serum Albumin, BRCA2 (aa 1323-1346), |
| | BrdU, Bromodeoxyuridine (BrdU), CA125, CA19-9, c-Abl, Cadherin (Pan), Cadherin-E, |
| | Cadherin-P, Calcitonin, Calcium Pump ATPase, Caldesmon, Calmodulin, Calponin, |
| | Calretinin, Casein, Caspase 1, Caspase 2, Caspase 3, Caspase 5, Caspase 6 (Mch 2), Caspase |
| | 7 (Mch 3), Caspase 8 (FLICE), Caspase 9, Catenin alpha, Catenin beta, Catenin gamma, |
| | Cathepsin D, CCK-8, CD1, CD10, CD100/Leukocyte Semaphorin, CD105, CD106/VCAM, |
| | CD115/c-fms/CSF-1R/M-CSFR, CD137 (4-1BB), CD138, CD14, CD15, CD155/PVR (Polio |
| | Virus Receptor), CD16, CD165, CD18, CD1a, CD1b, CD2, CD20, CD21, CD23, CD231, |
| | CD24, CD25/IL-2 Receptor a, CD26/DPP IV, CD29, CD30 (Reed-Sternberg Cell Marker), |
| | CD32/Fcg Receptor II, CD35/CR1, CD36GPIIIb/GPIV, CD3zeta, CD4, CD40, CD42b, |
| | CD43, CD45/T200/LCA, CD45RB, CD45RO, CD46, CD5, CD50/ICAM-3, CD53, |
| | CD54/ICAM-1, CD56/NCAM-1, CD57, CD59/MACIF/MIRL/Protectin, CD6, CD61/ |
| | Platelet Glycoprotein IIIA, CD63, CD68, CD71/Transferrin Receptor, CD79a mb-1, CD79b, |
| | CD8, CD81/TAPA-1, CD84, CD9, CD94, CD95/Fas, CD98, CDC14A Phosphatase, |
| | CDC25C, CDC34, CDC37, CDC47, CDC6, cdh1, Cdk1/p34cdc2, Cdk2, Cdk3, Cdk4, Cdk5, |
| | Cdk7, Cdk8, CDw17, CDw60, CDw75, CDw78, CEA/CD66e, c-erbB-2/HER-2/neu Ab-1 |
| | (21N), c-erbB-4/HER-4, c-fos, Chk1, Chorionic Gonadotropin beta (hCG-beta), |
| | Chromogranin A, CIDE-A, CIDE-B, CITED1, c-jun, Clathrin, claudin 11, Claudin 2, Claudin |
| | 3, Claudin 4, Claudin 5, CLAUDIN 7, Claudin-1, CNPase, Collagen II, Collagen IV, |
| | Collagen IX, Collagen VII, Connexin 43, COX2, CREB, CREB-Binding Protein, |
| | Cryptococcus neoformans, c-Src, Cullin-1 (CUL-1), Cullin-2 (CUL-2), Cullin-3 (CUL-3), |
| | CXCR4/Fusin, Cyclin B1, Cyclin C, Cyclin D1, Cyclin D3, Cyclin E, Cyclin E2, Cystic |
| | Fibrosis Transmembrane Regulator, Cytochrome c, D4-GDI, Daxx, DcR1, DcR2/TRAIL- |
| | R4/TRUNDD, Desmin, DFF40 (DNA Fragmentation Factor 40)/CAD, DFF45/ICAD, |
| | DJ-1, DNA Ligase I, DNA Polymerase Beta, DNA Polymerase Gamma, DNA Primase (p49), |
| | DNA Primase (p58), DNA-PKcs, DP-2, DR3, DRS, Dysferlin, Dystrophin, E2F-1, E2F-2, |
| | E2F-3, E2F-4, E2F-5, E3-binding protein (ARM1), EGFR, EMA/CA15-3/MUC-1, |
| | Endostatin, Epithelial Membrane Antigen (EMA/CA15-3/MUC-1), Epithelial Specific |
| | Antigen, ER beta, ER Ca+2 ATPase2, ERCC1, Erk1, ERK2, Estradiol, Estriol, Estrogen |
| | Receptor, Exo1, Ezrin/p81/80K/Cytovillin, F.VIII/VWF, Factor VIII Related Antigen, FADD |
| | (FAS-Associated death domain-containing protein), Fascin, Fas-ligand, Ferritin, FGF-1, |
| | FGF-2, FHIT, Fibrillin-1, Fibronectin, Filaggrin, Filamin, FITC, Fli-1, FLIP, Flk-1/KDR/ |
| | VEGFR2, Flt-1/VEGFR1, Flt-4, Fra2, FSH, FSH-b, Fyn, Ga0, Gab-1, GABA a Receptor 1, |
| | GAD65, Gai1, Gamma Glutamyl Transferase (gGT), Gamma Glutamylcysteine |
| | Synthetase(GCS)/Glutamate-cysteine Ligase, GAPDH, Gastrin 1, GCDFP-15, G-CSF, |
| | GFAP, Glicentin, Glucagon, Glucose-Regulated Protein 94, GluR 2/3, GluR1, GluR4, |
| | GluR6/7, GLUT-1, GLUT-3, Glycogen Synthase Kinase 3b (GSK3b), Glycophorin A, GM- |
| | CSF, GnRH Receptor, Golgi Complex, Granulocyte, Granzyme B, Grb2, Green Fluorescent |
| | Protein (GFP), GRIP1, Growth Hormone (hGH), GSK-3, GST, GSTmu, H. Pylori, HDAC1, |
| | HDJ-2/DNAJ, Heat Shock Factor 1, Heat Shock Factor 2, Heat Shock Protein 27/hsp27, Heat |
| | Shock Protein 60/hsp60, Heat Shock Protein 70/hsp70, Heat Shock Protein 75/hsp75, Heat |
| | Shock Protein 90a/hsp86, Heat Shock Protein 90b/hsp84, Helicobacter pylori, Heparan |
| | Sulfate Proteoglycan, Hepatic Nuclear Factor-3B, Hepatocyte, Hepatocyte Factor |
| | Homologue-4, Hepatocyte Growth Factor, Heregulin, HIF-1a, Histone H1, hPL, HPV 16, |
| | HPV 16-E7, HRP, Human Sodium Iodide Symporter (hNIS), I-FLICE/CASPER, IFN |
| | gamma, IgA, IGF-1R, IGF-I, IgG, IgM (m-Heavy Chain), I-Kappa-B Kinase b (IKKb), IL-1 |
| | alpha, IL-1 beta, IL-10, IL-10R, IL17, IL-2, IL-3, IL-30, IL-4, IL-5, IL-6, IL-8, Inhibin alpha, |
| | Insulin, Insulin Receptor, Insulin Receptor Substrate-1, Int-2 Oncoprotein, Integrin beta5, |
| | Interferon-a(II), Interferon-g, Involucrin, IP10/CRG2, IPO-38 Proliferation Marker, IRAK, |
| | ITK, JNK Activating kinase (JKK1), Kappa Light Chain, Keratin 10, Keratin 10/13, Keratin |
| | 14, Keratin 15, Keratin 16, Keratin 18, Keratin 19, Keratin 20, Keratin 5/6/18, Keratin 5/8, |
| | Keratin 8, Keratin 8 (phospho-specific Ser73), Keratin 8/18, Keratin (LMW), Keratin (Multi), |
| | Keratin (Pan), Ki67, Ku (p70/p80), Ku (p80), L1 Cell Adhesion Molecule, Lambda Light |
| | Chain, Laminin B1/b1, Laminin B2/g1, Laminin Receptor, Laminin-s, Lck, Lck (p56lck), |
| | Leukotriene (C4, D4, E4), LewisA, LewisB, LH, L-Plastin, LRP/MVP, Luciferase, |
| | Macrophage, MADD, MAGE-1, Maltose Binding Protein, MAP1B, MAP2a,b, MART- |
| | 1/Melan-A, Mast Cell Chymase, Mcl-1, MCM2, MCM5, MDM2, Medroxyprogesterone |
| | Acetate (MPA), Mek1, Mek2, Mek6, Mekk-1, Melanoma (gp100), mGluR1, mGluR5, |
| | MGMT, MHC I (HLA25 and HLA-Aw32), MHC I (HLA-A), MHC I (HLA-A, B, C), MHC I |
| | (HLA-B), MHC II (HLA-DP and DR), MHC II (HLA-DP), MHC II (HLA-DQ), MHC II |
| | (HLA-DR), MHC II (HLA-DR) Ia, Microphthalmia, Milk Fat Globule Membrane Protein, |
| | Mitochondria, MLH1, MMP-1 (Collagenase-I), MMP-10 (Stromilysin-2), MMP-11 |
| | (Stromelysin-3), MMP-13 (Collagenase-3), MMP-14/MT1-MMP, MMP-15/MT2-MMP, |
| | MMP-16/MT3-MMP, MMP-19, MMP-2 (72 kDa Collagenase IV), MMP-23, MMP-7 |
| | (Matrilysin), MMP-9 (92 kDa Collagenase IV), Moesin, mRANKL, Muc-1, Mucin 2, Mucin 3 |
| | (MUC3), Mucin 5AC, MyD88, Myelin/Oligodendrocyte, Myeloid Specific Marker, |
| | Myeloperoxidase, MyoD1, Myogenin, Myoglobin, Myosin Smooth Muscle Heavy Chain, |
| | Nck, Negative Control for Mouse IgG1, Negative Control for Mouse IgG2a, Negative |
| | Control for Mouse IgG3, Negative Control for Mouse IgM, Negative Control for Rabbit IgG, |
| | Neurofilament, Neurofilament (160 kDa), Neurofilament (200 kDa), Neurofilament (68 kDa), |
| | Neuron Specific Enolase, Neutrophil Elastase, NF kappa B/p50, NF kappa B/p65 (Rel A), |
| | NGF-Receptor (p75NGFR), brain Nitric Oxide Synthase (bNOS), endothelial Nitric Oxide |
| | Synthase (eNOS), nm23, NOS-i, NOS-u, Notch, Nucleophosmin (NPM), NuMA, Oct-1, |
| | Oct-2/, Oct-3/, Ornithine Decarboxylase, Osteopontin, p130, p130cas, p14ARF, p15INK4b, |
| | p16INK4a, p170, p170/MDR-1, p18INK4c, p19ARF, p19Skp1, p21WAF1, p27Kip1, p300/ |
| | CBP, p35nck5a, P504S, p53, p57Kip2 Ab-7, p63 (p53 Family Member), p73, p73a, p73a/b, |
| | p95VAV, Parathyroid Hormone, Parathyroid Hormone Receptor Type 1, Parkin, PARP, |
| | PARP (Poly ADP-Ribose Polymerase), Pax-5, Paxillin, PCNA, PCTAIRE2, PDGF, PDGFR |
| | alpha, PDGFR beta, Pds1, Perforin, PGP9.5, PHAS-I, PHAS-II, Phospho-Ser/Thr/Tyr, |
| | Phosphotyrosine, PLAP, Plasma Cell Marker, Plasminogen, PLC gamma 1, PMP-22, |
| | Pneumocystis jiroveci, PPAR-gamma, PR3 (Proteinase 3), Presenillin, Progesterone, |
| | Progesterone Receptor, Progesterone Receptor (phospho-specific) - Serine 190, Progesterone |
| | Receptor (phospho-specific) - Serine 294, Prohibitin, Prolactin, Prolactin Receptor, Prostate |
| | Apoptosis Response Protein-4, Prostate Specific Acid Phosphatase, Prostate Specific |
| | Antigen, pS2, PSCA, Rabies Virus, RAD1, Rad51, Raf1, Raf-1 (Phospho-specific), RAIDD, |
| | Ras, Rad18, Renal Cell Carcinoma, Ret Oncoprotein, Retinoblastoma, Retinoblastoma (Rb) |
| | (Phospho-specific Serine608), Retinoic Acid Receptor (b), Retinoid X Receptor (hRXR), |
| | Retinol Binding Protein, Rhodopsin (Opsin), ROC, RPA/p32, RPA/p70, Ruv A, Ruv B, Ruv |
| | C, S100, S100A4, S100A6, SHP-1, SIM Ag (SIMA-4D3), SIRP a1, sm, SODD (Silencer of |
| | Death Domain), Somatostatin Receptor-I, SRC1 (Steroid Receptor Coactivator-1) Ab-1, |
| | SREBP-1 (Sterol Regulatory Element Binding Protein-1), SRF (Serum Response Factor), |
| | Stat-1, Stat3, Stat5, Stat5a, Stat5b, Stat6, Streptavidin, Superoxide Dismutase, Surfactant |
| | Protein A, Surfactant Protein B, Surfactant Protein B (Pro), Survivin, SV40 Large T Antigen, |
| | Syk, Synaptophysin, Synuclein, Synuclein beta, Synuclein pan, TACE (TNF-alpha |
| | converting enzyme)/ADAM17, TAG-72, tau, TdT, Tenascin, Testosterone, TGF beta 3, |
| | TGF-beta 2, Thomsen-Friedenreich Antigen, Thrombospondin, Thymidine Phosphorylase, |
| | Thymidylate Synthase, Thymine Glycols, Thyroglobulin, Thyroid Hormone Receptor beta, |
| | Thyroid Hormone Receptor, Thyroid Stimulating Hormone (TSH), TID-1, TIMP-1, TIMP-2, |
| | TNF alpha, TNFa, TNR-R2, Topo II beta, Topoisomerase IIa, Toxoplasma Gondii, TR2, |
| | TRADD, Transforming Growth Factor a, Transglutaminase II, TRAP, Tropomyosin, TRP75/ |
| | gp75, TrxR2, TTF-1, Tubulin, Tubulin-a, Tubulin-b, Tyrosinase, Ubiquitin, UCP3, uPA, |
| | Urocortin, Vacular Endothelial Growth Factor(VEGF), Vimentin, Vinculin, Vitamin D |
| | Receptor (VDR), von Hippel-Lindau Protein, Wnt-1, Xanthine Oxidase, XPA, XPF, XPG, |
| | XRCC1, XRCC2, ZAP-70, Zip kinase |
| Known Cancer | ABL1, ABL2, ACSL3, AF15Q14, AF1Q, AF3p21, AF5q31, AKAP9, AKT1, AKT2, |
| Genes | ALDH2, ALK, ALO17, APC, ARHGEF12, ARHH, ARID1A, ARID2, ARNT, ASPSCR1, |
| | ASXL1, ATF1, ATIC, ATM, ATRX, BAP1, BCL10, BCL11A, BCL11B, BCL2, BCL3, |
| | BCL5, BCL6, BCL7A, BCL9, BCOR, BCR, BHD, BIRC3, BLM, BMPR1A, BRAF, |
| | BRCA1, BRCA2, BRD3, BRD4, BRIP1, BTG1, BUB1B, C12orf9, C15orf21, C15orf55, |
| | C16orf75, CANT1, CARD11, CARS, CBFA2T1, CBFA2T3, CBFB, CBL, CBLB, CBLC, |
| | CCNB1IP1, CCND1, CCND2, CCND3, CCNE1, CD273, CD274, CD74, CD79A, CD79B, |
| | CDH1, CDH11, CDK12, CDK4, CDK6, CDKN2A, CDKN2a(p14), CDKN2C, CDX2, |
| | CEBPA, CEP1, CHCHD7, CHEK2, CHIC2, CHN1, CIC, CIITA, CLTC, CLTCL1, |
| | CMKOR1, COL1A1, COPEB, COX6C, CREB1, CREB3L1, CREB3L2, CREBBP, CRLF2, |
| | CRTC3, CTNNB1, CYLD, D10S170, DAXX, DDB2, DDIT3, DDX10, DDX5, DDX6, |
| | DEK, DICER1, DNMT3A, DUX4, EBF1, EGFR, EIF4A2, ELF4, ELK4, ELKS, ELL, ELN, |
| | EML4, EP300, EPS15, ERBB2, ERCC2, ERCC3, ERCC4, ERCC5, ERG, ETV1, ETV4, |
| | ETV5, ETV6, EVI1, EWSR1, EXT1, EXT2, EZH2, FACL6, FAM22A, FAM22B, FAM46C, |
| | FANCA, FANCC, FANCD2, FANCE, FANCF, FANCG, FBXO11, FBXW7, FCGR2B, |
| | FEV, FGFR1, FGFR1OP, FGFR2, FGFR3, FH, FHIT, FIP1L1, FLI1, FLJ27352, FLT3, |
| | FNBP1, FOXL2, FOXO1A, FOXO3A, FOXP1, FSTL3, FUBP1, FUS, FVT1, GAS7, |
| | GATA1, GATA2, GATA3, GMPS, GNA11, GNAQ, GNAS, GOLGA5, GOPC, GPC3, |
| | GPHN, GRAF, HCMOGT-1, HEAB, HERPUD1, HEY1, HIP1, HIST1H4I, HLF, HLXB9, |
| | HMGA1, HMGA2, HNRNPA2B1, HOOK3, HOXA11, HOXA13, HOXA9, HOXC11, |
| | HOXC13, HOXD11, HOXD13, HRAS, HRPT2, HSPCA, HSPCB, IDH1, IDH2, IGH@, |
| | IGK@, IGL@, IKZF1, IL2, IL21R, IL6ST, IL7R, IRF4, IRTA1, ITK, JAK1, JAK2, JAK3, |
| | JAZF1, JUN, KDM5A, KDM5C, KDM6A, KDR, KIAA1549, KIT, KLK2, KRAS, KTN1, |
| | LAF4, LASP1, LCK, LCP1, LCX, LHFP, LIFR, LMO1, LMO2, LPP, LYL1, MADH4, |
| | MAF, MAFB, MALT1, MAML2, MAP2K4, MDM2, MDM4, MDS1, MDS2, MECT1, |
| | MED12, MEN1, MET, MITF, MKL1, MLF1, MLH1, MLL, MLL2, MLL3, MLLT1, |
| | MLLT10, MLLT2, MLLT3, MLLT4, MLLT6, MLLT7, MN1, MPL, MSF, MSH2, MSH6, |
| | MSI2, MSN, MTCP1, MUC1, MUTYH, MYB, MYC, MYCL1, MYCN, MYD88, MYH11, |
| | MYH9, MYST4, NACA, NBS1, NCOA1, NCOA2, NCOA4, NDRG1, NF1, NF2, NFE2L2, |
| | NFIB, NFKB2, NIN, NKX2-1, NONO, NOTCH1, NOTCH2, NPM1, NR4A3, NRAS, |
| | NSD1, NTRK1, NTRK3, NUMA1, NUP214, NUP98, OLIG2, OMD, P2RY8, PAFAH1B2, |
| | PALB2, PAX3, PAX5, PAX7, PAX8, PBRM1, PBX1, PCM1, PCSK7, PDE4DIP, PDGFB, |
| | PDGFRA, PDGFRB, PER1, PHOX2B, PICALM, PIK3CA, PIK3R1, PIM1, PLAG1, PML, |
| | PMS1, PMS2, PMX1, PNUTL1, POU2AF1, POU5F1, PPARG, PPP2R1A, PRCC, PRDM1, |
| | PRDM16, PRF1, PRKAR1A, PRO1073, PSIP2, PTCH, PTEN, PTPN11, RAB5EP, |
| | RAD51L1, RAF1, RALGDS, RANBP17, RAP1GDS1, RARA, RB1, RBM15, RECQL4, |
| | REL, RET, ROS1, RPL22, RPN1, RUNDC2A, RUNX1, RUNXBP2, SBDS, SDH5, SDHB, |
| | SDHC, SDHD, SEPT6, SET, SETD2, SF3B1, SFPQ, SFRS3, SH3GL1, SIL, SLC45A3, |
| | SMARCA4, SMARCB1, SMO, SOCS1, SOX2, SRGAP3, SRSF2, SS18, SS18L1, |
| | SSH3BP1, SSX1, SSX2, SSX4, STK11, STL, SUFU, SUZ12, SYK, TAF15, TAL1, TAL2, |
| | TCEA1, TCF1, TCF12, TCF3, TCF7L2, TCL1A, TCL6, TET2, TFE3, TFEB, TFG, TFPT, |
| | TFRC, THRAP3, TIF1, TLX1, TLX3, TMPRSS2, TNFAIP3, TNFRSF14, TNFRSF17, |
| | TNFRSF6, TOP1, TP53, TPM3, TPM4, TPR, TRA@, TRB@, TRD@, TRIM27, TRIM33, |
| | TRIP11, TSC1, TSC2, TSHR, TTL, U2AF1, USP6, VHL, VTI1A, WAS, WHSC1, |
| | WHSC1L1, WIF1, WRN, WT1, WTX, XPA, XPC, XPO1, YWHAE, ZNF145, ZNF198, |
| | ZNF278, ZNF331, ZNF384, ZNF521, ZNF9, ZRSR2 |
| Known Cancer | AR, androgen receptor; ARPC1A, actin-related protein complex 2/3 subunit A; AURKA, |
| Genes | Aurora kinase A; BAG4, BCl-2 associated anthogene 4; BCl2l2, BCl-2 like 2; BIRC2, |
| | Baculovirus IAP repeat containing protein 2; CACNA1E, calcium channel voltage dependent |
| | alpha-1E subunit; CCNE1, cyclin E1; CDK4, cyclin dependent kinase 4; CHD1L, |
| | chromodomain helicase DNA binding domain 1-like; CKS1B, CDC28 protein kinase 1B; |
| | COPS3, COP9 subunit 3; DCUN1D1, DCN1 domain containing protein 1; DYRK2, dual |
| | specificity tyrosine phosphorylation regulated kinase 2; EEF1A2, eukaryotic elongation |
| | transcription factor 1 alpha 2; EGFR, epidermal growth factor receptor; FADD, Fas- |
| | associated via death domain; FGFR1, fibroblast growth factor receptor 1, GATA6, GATA |
| | binding protein 6; GPC5, glypican 5; GRB7, growth factor receptor bound protein 7; |
| | MAP3K5, mitogen activated protein kinase kinase kinase 5; MED29, mediator complex |
| | subunit 5; MITF, microphthalmia associated transcription factor; MTDH, metadherin; |
| | NCOA3, nuclear receptor coactivator 3; NKX2-1, NK2 homeobox 1; PAK1, |
| | p21/CDC42/RAC1-activated kinase 1; PAX9, paired box gene 9; PIK3CA, |
| | phosphatidylinositol-3 kinase catalytic a; PLA2G10, phopholipase A2, group X; PPM1D, |
| | protein phosphatase magnesium-dependent 1D; PTK6, protein tyrosine kinase 6; PRKCI, |
| | protein kinase C iota; RPS6KB1, ribosomal protein s6 kinase 70 kDa; SKP2, s-phase kinase |
| | associated protein; SMURF1, sMAD specific E3 ubiquitin protein ligase 1; SHH, sonic |
| | hedgehog homologue; STARD3, sTAR-related lipid transfer domain containing protein 3; |
| | YWHAQ, tyrosine 3-monooxygenase/tryptophan 5-monooxygenase activation protein, zeta |
| | isoform; ZNF217, zinc finger protein 217 |
| Mitotic Related | Aurora kinase A (AURKA); Aurora kinase B (AURKB); Baculoviral IAP repeat-containing |
| Cancer Genes | 5, survivin (BIRC5); Budding uninhibited by benzimidazoles 1 homolog (BUB1); Budding |
| | uninhibited by benzimidazoles 1 homolog beta, BUBR1 (BUB1B); Budding uninhibited by |
| | benzimidazoles 3 homolog (BUB3); CDC28 protein kinase regulatory subunit 1B (CKS1B); |
| | CDC28 protein kinase regulatory subunit 2 (CKS2); Cell division cycle 2 (CDC2)/CDK1 |
| | Cell division cycle 20 homolog (CDC20); Cell division cycle-associated 8, borealin |
| | (CDCA8); Centromere protein F, mitosin (CENPF); Centrosomal protein 110 kDa (CEP110); |
| | Checkpoint with forkhead and ring finger domains (CHFR); Cyclin B1 (CCNB1); Cyclin B2 |
| | (CCNB2); Cytoskeleton-associated protein 5 (CKAP5/ch-TOG); Microtubule-associated |
| | protein RP/EB family member 1. End-binding protein 1, EB1 (MAPRE1); Epithelial cell |
| | transforming sequence 2 oncogene (ECT2); Extra spindle poles like 1, separase (ESPL1); |
| | Forkhead box M1 (FOXM1); H2A histone family, member X (H2AFX); Kinesin family |
| | member 4A (KIF4A); Kinetochore-associated 1 (KNTC1/ROD); Kinetochore-associated 2; |
| | highly expressed in cancer 1 (KNTC2/HEC1); Large tumor suppressor, homolog 1 (LATS1); |
| | Large tumor suppressor, homolog 2 (LATS2); Mitotic arrest deficient-like 1; MAD1 |
| | (MAD1L1); Mitotic arrest deficient-like 2; MAD2 (MAD2L1); Mps1 protein kinase (TTK); |
| | Never in mitosis gene a-related kinase 2 (NEK2); Ninein, GSK3b interacting protein (NIN); |
| | Non-SMC condensin I complex, subunit D2 (NCAPD2/CNAP1); Non-SMC condensin I |
| | complex, subunit H (NACPH/CAPH); Nuclear mitotic apparatus protein 1 (NUMA1); |
| | Nucleophosmin (nucleolar phosphoprotein B23, numatrin); (NPM1); Nucleoporin (NUP98); |
| | Pericentriolar material 1 (PCM1); Pituitary tumor-transforming 1, securin (PTTG1); Polo-like |
| | kinase 1 (PLK1); Polo-like kinase 4 (PLK4/SAK); Protein (peptidylprolyl cis/trans |
| | isomerase) NIMA-interacting 1 (PIN1); Protein regulator of cytokinesis 1 (PRC1); RAD21 |
| | homolog (RAD21); Ras association (RalGDS/AF-6); domain family 1 (RASSF1); Stromal |
| | antigen 1 (STAG1); Synuclein-c, breast cancer-specific protein 1 (SNCG, BCSG1); |
| | Targeting protein for Xklp2 (TPX2); Transforming, acidic coiled-coil containing protein 3 |
| | (TACC3); Ubiquitin-conjugating enzyme E2C (UBE2C); Ubiquitin-conjugating enzyme E2I |
| | (UBE2I/UBC9); ZW10 interactor, (ZWINT); ZW10, kinetochore-associated homolog |
| | (ZW10); Zwilch, kinetochore-associated homolog (ZWILCH) |
| Ribonucleoprotein | Argonaute family member, Ago1, Ago2, Ago3, Ago4, GW182 (TNRC6A), TNRC6B, |
| complexes | TNRC6C, HNRNPA2B1, HNRPAB, ILF2, NCL (Nucleolin), NPM1 (Nucleophosmin), |
| | RPL10A, RPL5, RPLP1, RPS12, RPS19, SNRPG, TROVE2, apolipoprotein, apolipoprotein |
| | A, apo A-I, apo A-II, apo A-IV, apo A-V, apolipoprotein B, apo B48, apo B100, |
| | apolipoprotein C, apo C-I, apo C-II, apo C-III, apo C-IV, apolipoprotein D (ApoD), |
| | apolipoprotein E (ApoE), apolipoprotein H (ApoH), apolipoprotein L, APOL1, APOL2, |
| | APOL3, APOL4, APOL5, APOL6, APOLD1 |
| Cytokine Receptors | 4-1BB, ALCAM, B7-1, BCMA, CD14, CD30, CD40 Ligand, CEACAM-1, DR6, Dtk, |
| | Endoglin, ErbB3, E-Selectin, Fas, Flt-3L, GITR, HVEM, ICAM-3, IL-1 R4, IL-1 RI, IL-10 |
| | Rbeta, IL-17R, IL-2Rgamma, IL-21R, LIMPII, Lipocalin-2, L-Selectin, LYVE-1, MICA, |
| | MICB, NRG1-beta1, PDGF Rbeta, PECAM-1, RAGE, TIM-1, TRAIL R3, Trappin-2, uPAR, |
| | VCAM-1, XEDAR |
| Prostate and | ErbB3, RAGE, Trail R3 |
| colorectal cancer | |
| vesicles | |
| Colorectal cancer | IL-1 alpha, CA125, Filamin, Amyloid A |
| vesicles | |
| Colorectal cancer v | Involucrin, CD57, Prohibitin, Thrombospondin, Laminin B1/b1, Filamin, 14.3.3 gamma, |
| adenoma vesicles | 14.3.3 Pan |
| Colorectal | Involucrin, Prohibitin, Laminin B1/b1, IL-3, Filamin, 14.3.3 gamma, 14.3.3 Pan, MMP-15/ |
| adenoma vesicles | MT2-MMP, hPL, Ubiquitin, and mRANKL |
| Brain cancer | Prohibitin, CD57, Filamin, CD18, b-2-Microglobulin, IL-2, IL-3, CD16, p170, Keratin 19, |
| vesicles | Pds1, Glicentin, SRF (Serum Response Factor), E3-binding protein (ARM1), Collagen II, |
| | SRC1 (Steroid Receptor Coactivator-1) Ab-1, Caldesmon, GFAP, TRP75/gp75, alpha-1- |
| | antichymotrypsin, Hepatic Nuclear Factor-3B, PLAP, Tyrosinase, NF kappa B/p50, |
| | Melanoma (gp100), Cyclin E, 6-Histidine, Mucin 3 (MUC3), TdT, CD21, XPA, Superoxide |
| | Dismutase, Glycogen Synthase Kinase 3b (GSK3b), CD54/ICAM-1, Thrombospondin, Gai1, |
| | CD79a mb-1, IL-1 beta, Cytochrome c, RAD1, bcl-X, CD50/ICAM-3, Neurofilament, |
| | Alkaline Phosphatase (AP), ER Ca+2 ATPase2, PCNA, F.VIII/VWF, SV40 Large T Antigen, |
| | Paxillin, Fascin, CD165, GRIP1, Cdk8, Nucleophosmin (NPM), alpha-1-antitrypsin, |
| | CD32/Fcg Receptor II, Keratin 8 (phospho-specific Ser73), DR5, CD46, TID-1, MHC II |
| | (HLA-DQ), Plasma Cell Marker, DR3, Calmodulin, AIF (Apoptosis Inducing Factor), DNA |
| | Polymerase Beta, Vitamin D Receptor (VDR), Bcl10/CIPER/CLAP/mE10, Neuron |
| | Specific Enolase, CXCR4/Fusin, Neurofilament (68 kDa), PDGFR, beta, Growth Hormone |
| | (hGH), Mast Cell Chymase, Ret Oncoprotein, and Phosphotyrosine |
| Melanoma vesicles | Caspase 5, Thrombospondin, Filamin, Ferritin, 14.3.3 gamma, 14.3.3 Pan, CD71/Transferrin |
| | Receptor, and Prostate Apoptosis Response Protein-4 |
| Head and neck | 14.3.3 Pan, Filamin, 14.3.3 gamma, CD71/Transferrin Receptor, CD30, Cdk5, CD138, |
| cancer vesicles | Thymidine Phosphorylase, Ruv 5, Thrombospondin, CD1, Von Hippel-Lindau Protein, |
| | CD46, Rad51, Ferritin, c-Abl, Actin, Muscle Specific, LewisB |
| Membrane proteins | carbonic anhydrase IX, B7, CCCL19, CCCL21, CSAp, HER-2/neu, BrE3, CD1, CD1a, CD2, |
| | CD3, CD4, CD5, CD8, CD11A, CD14, CD15, CD16, CD18, CD19, CD20, CD21, CD22, |
| | CD23, CD25, CD29, CD30, CD32b, CD33, CD37, CD38, CD40, CD40L, CD44, CD45, |
| | CD46, CD52, CD54, CD55, CD59, CD64, CD67, CD70, CD74, CD79a, CD80, CD83, |
| | CD95, CD126, CD133, CD138, CD147, CD154, CEACAM5, CEACAM-6, alpha-fetoprotein |
| | (AFP), VEGF, ED-B fibronectin, EGP-1, EGP-2, EGF receptor (ErbB1), ErbB2, ErbB3, |
| | Factor H, FHL-1, Flt-3, folate receptor, Ga 733, GROB, HMGB-1, hypoxia inducible factor |
| | (HIF), HM1.24, HER-2/neu, insulin-like growth factor (ILGF), IFN-γ, IFN-α, IL-β, IL-2R, |
| | IL-4R, IL-6R, IL-13R, IL-15R, IL-17R, IL-18R, IL-2, IL-6, IL-8, IL-12, IL-15, IL-17, IL-18, |
| | IL-25, IP-10, IGF-1R, Ia, HM1.24, gangliosides, HCG, HLA-DR, CD66a-d, MAGE, mCRP, |
| | MCP-1, MIP-1A, MIP-1B, macrophage migration-inhibitory factor (MIF), MUC1, MUC2, |
| | MUC3, MUC4, MUC5, placental growth factor (P1GF), PSA (prostate-specific antigen), |
| | PSMA, PSMA dimer, PAM4 antigen, NCA-95, NCA-90, A3, A33, Ep-CAM, KS-1, Le(y), |
| | mesothelin, S100, tenascin, TAC, Tn antigen, Thomas-Friedenreich antigens, tumor necrosis |
| | antigens, tumor angiogenesis antigens, TNF-α, TRAIL receptor (R1 and R2), VEGFR, |
| | RANTES, T101, cancer stem cell antigens, complement factors C3, C3a, C3b, C5a, C5 |
| Cluster of | CD1, CD2, CD3, CD4, CD5, CD6, CD7, CD8, CD9, CD10, CD11a, CD11b, CD11c, |
| Differentiation | CD12w, CD13, CD14, CD15, CD16, CDw17, CD18, CD19, CD20, CD21, CD22, CD23, |
| (CD) proteins | CD24, CD25, CD26, CD27, CD28, CD29, CD30, CD31, CD32, CD33, CD34, CD35, CD36, |
| | CD37, CD38, CD39, CD40, CD41, CD42, CD43, CD44, CD45, CD46, CD47, CD48, |
| | CD49a, CD49b, CD49c, CD49d, CD49e, CD49f, CD53, CD54, CD55, CD56, CD57, CD58, |
| | CD59, CD61, CD62E, CD62L, CD62P, CD63, CD68, CD69, CD71, CD72, CD73, CD74, |
| | CD80, CD81, CD82, CD83, CD86, CD87, CD88, CD89, CD90, CD91, CD95, CD96, |
| | CD100, CD103, CD105, CD106, CD107, CD107a, CD107b, CD109, CD117, CD120, |
| | CD127, CD133, CD134, CD135, CD138, CD141, CD142, CD143, CD144, CD147, CD151, |
| | CD152, CD154, CD156, CD158, CD163, CD165, CD166, CD168, CD184, CDw186, |
| | CD195, CD197, CD209, CD202a, CD220, CD221, CD235a, CD271, CD303, CD304, |
| | CD309, CD326 |
| Interleukin (IL) | IL-1, IL-2, IL-3, IL-4, IL-5, IL-6, IL-7, IL-8 or CXCL8, IL-9, IL-10, IL-11, IL-12, IL-13, IL- |
| proteins | 14, IL-15, IL-16, IL-17, IL-18, IL-19, IL-20, IL-21, IL-22, IL-23, IL-24, IL-25, IL-26, IL-27, |
| | IL-28, IL-29, IL-30, IL-31, IL-32, IL-33, IL-35, IL-36 |
| IL receptors | CD121a/IL1R1, CD121b/IL1R2, CD25/IL2RA, CD122/IL2RB, CD132/IL2RG, |
| | CD123/IL3RA, CD131/IL3RB, CD124/IL4R, CD132/IL2RG, CD125/IL5RA, |
| | CD131/IL3RB, CD126/IL6RA, CD130/IR6RB, CD127/IL7RA, CD132/IL2RG, |
| | CXCR1/IL8RA, CXCR2/IL8RB/CD128, CD129/IL9R, CD210/IL10RA, |
| | CDW210B/IL10RB, IL11RA, CD212/IL12RB1, IR12RB2, IL13R, IL15RA, CD4, |
| | CDw217/IL17RA, IL17RB, CDw218a/IL18R1, IL20R, IL20R, IL21R, IL22R, IL23R, |
| | IL20R, LY6E, IL20R1, IL27RA, IL28R, IL31RA |
| Mucin (MUC) | MUC1, MUC2, MUC3A, MUC3B, MUC4, MUC5AC, MUC5B, MUC6, MUC7, MUC8, |
| proteins | MUC12, MUC13, MUC15, MUC16, MUC17, MUC19, and MUC20 |
| MUC1 isoforms | mucin-1 isoform 2 precursor or mature form (NP_001018016.1), mucin-1 isoform 3 |
| | precursor or mature form (NP_001018017.1), mucin-1 isoform 5 precursor or mature form |
| | (NP_001037855.1), mucin-1 isoform 6 precursor or mature form (NP_001037856.1), mucin- |
| | 1 isoform 7 precursor or mature form (NP_001037857.1), mucin-1 isoform 8 precursor or |
| | mature form (NP_001037858.1), mucin-1 isoform 9 precursor or mature form |
| | (NP_001191214.1), mucin-1 isoform 10 precursor or mature form (NP_001191215.1), |
| | mucin-1 isoform 11 precursor or mature form (NP_001191216.1), mucin-1 isoform 12 |
| | precursor or mature form (NP_001191217.1), mucin-1 isoform 13 precursor or mature form |
| | (NP_001191218.1), mucin-1 isoform 14 precursor or mature form (NP_001191219.1), |
| | mucin-1 isoform 15 precursor or mature form (NP_001191220.1), mucin-1 isoform 16 |
| | precursor or mature form (NP_001191221.1), mucin-1 isoform 17 precursor or mature form |
| | (NP_001191222.1), mucin-1 isoform 18 precursor or mature form (NP_001191223.1), |
| | mucin-1 isoform 19 precursor or mature form (NP_001191224.1), mucin-1 isoform 20 |
| | precursor or mature form (NP_001191225.1), mucin-1 isoform 21 precursor or mature form |
| | (NP_001191226.1), mucin-1 isoform 1 precursor or mature form (NP_002447.4), |
| | ENSP00000357380, ENSP00000357377, ENSP00000389098, ENSP00000357374, |
| | ENSP00000357381, ENSP00000339690, ENSP00000342814, ENSP00000357383, |
| | ENSP00000357375, ENSP00000338983, ENSP00000343482, ENSP00000406633, |
| | ENSP00000388172, ENSP00000357378, P15941-1, P15941-2, P15941-3, P15941-4, |
| | P15941-5, P15941-6, P15941-7, P15941-8, P15941-9, P15941-10, secreted isoform, |
| | membrane bound isoform, CA 27.29 (BR 27.29), CA 15-3, PAM4 reactive antigen, |
| | underglycosylated isoform, unglycosylated isoform, CanAg antigen |
| MUC1 interacting | ABL1, SRC, CTNND1, ERBB2, GSK3B, JUP, PRKCD, APC, GALNT1, GALNT10, |
| proteins | GALNT12, JUN, LCK, OSGEP, ZAP70, CTNNB1, EGFR, SOS1, ERBB3, ERBB4, GRB2, |
| | ESR1, GALNT2, GALNT4, LYN, TP53, C1GALT1, C1GALT1C1, GALNT3, GALNT6, |
| | GCNT1, GCNT4, MUC12, MUC13, MUC15, MUC17, MUC19, MUC2, MUC20, MUC3A, |
| | MUC4, MUC5B, MUC6, MUC7, MUCL1, ST3GAL1, ST3GAL3, ST3GAL4, |
| | ST6GALNAC2, B3GNT2, B3GNT3, B3GNT4, B3GNT5, B3GNT7, B4GALT5, GALNT11, |
| | GALNT13, GALNT14, GALNT5, GALNT8, GALNT9, ST3GAL2, ST6GAL1, |
| | ST6GALNAC4, GALNT15, MYOD1, SIGLEC1, IKBKB, TNFRSF1A, IKBKG, MUC1 |
| Tumor markers | Alphafetoprotein (AFP), Carcinoembryonic antigen (CEA), CA-125, MUC-1, Epithelial |
| | tumor antigen (ETA), Tyrosinase, Melanoma-associated antigen (MAGE), p53 |
| Tumor markers | Alpha fetoprotein (AFP), CA15-3, CA27-29, CA19-9, CA-125, Calretinin, |
| | Carcinoembryonic antigen, CD34, CD99, CD117, Chromogranin, Cytokeratin (various |
| | types), Desmin, Epithelial membrane protein (EMA), Factor VIII, CD31 FL1, Glial fibrillary |
| | acidic protein (GFAP), Gross cystic disease fluid protein (GCDFP-15), HMB-45, Human |
| | chorionic gonadotropin (hCG), immunoglobulin, inhibin, keratin (various types), PTPRC |
| | (CD45), lymphocyte marker (various types, MART-1 (Melan-A), Myo D1, muscle-specific |
| | actin (MSA), neurofilament, neuron-specific enolase (NSE), placental alkaline phosphatase |
| | (PLAP), prostate-specific antigen, S100 protein, smooth muscle actin (SMA), synaptophysin, |
| | thyroglobulin, thyroid transcription factor-1, Tumor M2-PK, vimentin |
| Cell adhesion | Immunoglobulin superfamily CAMs (IgSF CAMs), N-CAM (Myelin protein zero), ICAM (1, |
| molecule (CAMs) | 5), VCAM-1, PE-CAM, L1-CAM, Nectin (PVRL1, PVRL2, PVRL3), Integrins, LFA-1 |
| | (CD11a + CD18), Integrin alphaXbeta2 (CD11c + CD18), Macrophage-1 antigen |
| | (CD11b + CD18), VLA-4 (CD49d + CD29), Glycoprotein IIb/IIIa (ITGA2B + ITGB3), |
| | Cadherins, CDH1, CDH2, CDH3, Desmosomal, Desmoglein (DSG1, DSG2, DSG3, DSG4), |
| | Desmocollin (DSC1, DSC2, DSC3), Protocadherin, PCDH1, T-cadherin, CDH4, CDH5, |
| | CDH6, CDH8, CDH11, CDH12, CDH15, CDH16, CDH17, CDH9, CDH10, Selectins, E- |
| | selectin, L-selectin, P-selectin, Lymphocyte homing receptor: CD44, L-selectin, integrin |
| | (VLA-4, LFA-1), Carcinoembryonic antigen (CEA), CD22, CD24, CD44, CD146, CD164 |
| Annexins | ANXA1; ANXA10; ANXA11; ANXA13; ANXA2; ANXA3; ANXA4; ANXA5; ANXA6; |
| | ANXA7; ANXA8; ANXA8L1; ANXA8L2; ANXA9 |
| Cadherins | CDH1, CDH2, CDH12, CDH3, Deomoglein, DSG1, DSG2, DSG3, DSG4, Desmocollin, |
| (“calcium- | DSC1, DSC2, DSC3, Protocadherins, PCDH1, PCDH10, PCDH11x, PCDH11y, PCDH12, |
| dependent | FAT, FAT2, FAT4, PCDH15, PCDH17, PCDH18, PCDH19; PCDH20; PCDH7, PCDH8, |
| adhesion”) | PCDH9, PCDHA1, PCDHA10, PCDHA11, PCDHA12, PCDHA13, PCDHA2, PCDHA3, |
| | PCDHA4, PCDHA5, PCDHA6, PCDHA7, PCDHA8, PCDHA9, PCDHAC1, PCDHAC2, |
| | PCDHB1, PCDHB10, PCDHB11, PCDHB12, PCDHB13, PCDHB14, PCDHB15, |
| | PCDHB16, PCDHB17, PCDHB18, PCDHB2, PCDHB3, PCDHB4, PCDHB5, PCDHB6, |
| | PCDHB7, PCDHB8, PCDHB9, PCDHGA1, PCDHGA10, PCDHGA11, PCDHGA12, |
| | PCDHGA2; PCDHGA3, PCDHGA4, PCDHGA5, PCDHGA6, PCDHGA7, PCDHGA8, |
| | PCDHGA9, PCDHGB1, PCDHGB2, PCDHGB3, PCDHGB4, PCDHGB5, PCDHGB6, |
| | PCDHGB7, PCDHGC3, PCDHGC4, PCDHGC5, CDH9 (cadherin 9, type 2 (T1-cadherin)), |
| | CDH10 (cadherin 10, type 2 (T2-cadherin)), CDH5 (VE-cadherin (vascular endothelial)), |
| | CDH6 (K-cadherin (kidney)), CDH7 (cadherin 7, type 2), CDH8 (cadherin 8, type 2), |
| | CDH11 (OB-cadherin (osteoblast)), CDH13 (T-cadherin-H-cadherin (heart)), CDH15 (M- |
| | cadherin (myotubule)), CDH16 (KSP-cadherin), CDH17 (LI cadherin (liver-intestine)), |
| | CDH18 (cadherin 18, type 2), CDH19 (cadherin 19, type 2), CDH20 (cadherin 20, type 2), |
| | CDH23 (cadherin 23, (neurosensory epithelium)), CDH10, CDH11, CDH13, CDH15, |
| | CDH16, CDH17, CDH18, CDH19, CDH20, CDH22, CDH23, CDH24, CDH26, CDH28, |
| | CDH4, CDH5, CDH6, CDH7, CDH8, CDH9, CELSR1, CELSR2, CELSR3, CLSTN1, |
| | CLSTN2, CLSTN3, DCHS1, DCHS2, LOC389118, PCLKC, RESDA1, RET |
| ECAD (CDH1) | SNAI1/SNAIL, ZFHX1B/SIP1, SNAI2/SLUG, TWIST1, DeltaEF1 |
| downregulators | |
| ECAD | AML1, p300, HNF3 |
| upregulators | |
| ECAD interacting | ACADVL, ACTG1, ACTN1, ACTN4, ACTR3, ADAM10, ADAM9, AJAP1, ANAPC1, |
| proteins | ANAPC11, ANAPC4, ANAPC7, ANK2, ANP32B, APC2, ARHGAP32, ARPC2, ARVCF, |
| | BOC, C1QBP, CA9, CASP3, CASP8, CAV1, CBLL1, CCNB1, CCND1, CCT6A, CDC16, |
| | CDC23, CDC26, CDC27, CDC42, CDH2, CDH3, CDK5R1, CDON, CDR2, CFTR, |
| | CREBBP, CSE1L, CSNK2A1, CTNNA1, CTNNB1, CTNND1, CTNND2, DNAJA1, DRG1, |
| | EGFR, EP300, ERBB2, ERBB2IP, ERG, EZR, FER, FGFR1, FOXM1, FRMD5, FYN, |
| | GBAS, GNA12, GNA13, GNB2L1, GSK3B, HDAC1, HDAC2, HSP90AA1, HSPA1A, |
| | HSPA1B, HSPD1, IGHA1, IQGAP1, IRS1, ITGAE, ITGB7, JUP, KIFC3, KLRG1, KRT1, |
| | KRT9, LIMA1, LMNA, MAD2L2, MAGI1, MAK, MDM2, MET, MYO6, MYO7A, |
| | NDRG1, NEDD9, NIPSNAP1, NKD2, PHLPP1, PIP5K1C, PKD1, PKP4, PLEKHA7, |
| | POLR2E, PPP1CA, PRKD1, PSEN1, PTPN1, PTPN14, PTPRF, PTPRM, PTPRQ, PTTG1, |
| | PVR, PVRL1, RAB8B, RRM2, SCRIB, SET, SIX1, SKI, SKP2, SRC, TACC3, TAS2R13, |
| | TGM2, TJP1, TK1, TNS3, TTK, UBC, USP9X, VCL, VEZT, XRCC5, YAP1, YES1, |
| | ZC3HC1 |
| Epithelial- | SERPINA3, ACTN1, AGR2, AKAP12, ALCAM, AP1M2, AXL, BSPRY, CCL2, CDH1, |
| mesenchymal | CDH2, CEP170, CLDN3, CLDN4, CNN3, CYP4X1, DNMT3A, DSG3, DSP, EFNB2, EHF, |
| transition (EMT) | ELF3, ELF5, ERBB3, ETV5, FLRT3, FOSB, FOSL1, FOXC1, FX YD 5, GPDIL, HMGA1, |
| | HMGA2, HOPX, IFI16, IGFBP2, IHH, IKBIP, IL-11, IL-18, IL6, IL8, ITGA5, ITGB3, |
| | LAMBl, LCN2, MAP7, MB, MMP7, MMP9, MPZL2, MSLN, MTA3, MTSS1, OCLN, |
| | PCOLCE2, PECAM1, PLAUR, PLXNB1, PPL, PPP1R9A, RASSF8, SCNN1A, SERPINB2, |
| | SERPINE1, SFRP1, SH3YL1, SLC27A2, SMAD7, SNAI1, SNAI2, SPARC, SPDEF, SRPX, |
| | STAT5A, TBX2, TJP3, TMEM125, TMEM45B, TWIST1, VCAN, VIM, VWF, XBP1, |
| | YBX1, ZBTB10, ZEB1, ZEB2 |

The instant disclosure provides various biomarkers that can be assessed in determining a biosignature for a given test sample, and which include assessment of polypeptides and/or nucleic acid biomarkers associated with various cancers, as well as the state of the cancer (e.g., metastatic v. non-metastatic).

In one example, a test sample can be assessed for a cancer by determining the presence or level of one or more biomarker including but not limited to CA-125, CA 19-9, and c-reactive protein. The cancer can be a cancer of the reproductive tract, e.g., an ovarian cancer. The one or more biomarker can further comprise one or more biomarkers, e.g., 1, 2, 3, 4, 5, 6, 7, 8, 9, 10, 11, 12, 13, 14, 15, 16, 17, 18, 19, 20 or more biomarkers, comprising one or more of CD95, FAP-1, miR-200 microRNAs, EGFR, EGFRvIII, apolipoprotein AI, apolipoprotein CIII, myoglobin, tenascin C, MSH6, claudin-3, claudin-4, caveolin-1, coagulation factor III, CD9, CD36, CD37, CD53, CD63, CD81, CD136, CD147, Hsp70, Hsp90, Rab13, Desmocollin-1, EMP-2, CK7, CK20, GCDF15, CD82, Rab-5b, Annexin V, MFG-E8 and HLA-DR. MiR-200 microRNAs (i.e., the miR-200 microRNA family) comprises miR-200a, miR-200b, miR-200c, miR-141 and miR-429. Such assessment can include determining the presence or levels of proteins, nucleic acids, or both for each of the biomarkers disclosed herein.

CD95 (also called Fas, Fas antigen, Fas receptor, FasR, TNFRSF6, APT1 or APO-1) is a prototypical death receptor that regulates tissue homeostasis mainly in the immune system through the induction of apoptosis. During cancer progression, CD95 is frequently downregulated and the cells are rendered apoptosis resistant, thereby implicating loss of CD95 as part of a mechanism for tumour evasion. The tumorigenic activity of CD95 is mediated by a pathway involving JNK and Jun. FAP-1 (also referred to as Fas-associated phosphatase 1, protein tyrosine phosphatase, non-receptor type 13 (APO-1/CD95 (Fas)-associated phosphatase), PTPN13) is a member of the protein tyrosine phosphatase (PTP) family. FAP-1 has been reported to interact with, and dephosphorylate, CD95, thereby implicating a role in Fas mediated programmed cell death. MiR-200 family members can regulate CD95 and FAP-1. See Schickel et al. miR-200c regulates induction of apoptosis through CD95 by targeting FAP-1. Mol. Cell., 38, 908-915 (2010), which publication is incorporated by reference in its entirety herein.

Methods of the invention disclosed herein can use CD95 and/or FAP-1 characterization or profiling for microvesicle populations present in a biological sample to determine the presence of or predisposition to cancer, including without limitation any of the cancers disclosed herein. Methods of the invention comprising multiplexed analysis for multiple biomarkers use CD95 and/or FAP-1 biomarker characterization, along with other biomarkers disclosed herein, including but not limited to miR-200 microRNAs (e.g., miR-200c). In an embodiment, a biological test sample from an individual is assessed to determine the presence and level of CD95 and/or FAP-1 protein, or a presence or level of a CD95+ and/or FAP-1+ circulating microvesicle (“cMV”) population, and the presence or levels are compared to a reference (e.g., samples from non-disease or normal, pre-treatment, or different treatment timepoints). This comparison is used to characterize the test sample. For example, comparison of the presence or levels of CD95 protein, FAP-1 protein, CD95+cMVs and/or FAP-1+cMVs in the test sample and reference are used to determine a disease phenotype or predict a response/non-response to treatment. In related embodiments, the cMV population is further assessed to determine a presence or level of miR-200 microRNAs, which are predetermined in a training set of reference samples to be indicative of disease or other prognostic, theranostic or diagnostic readout. Increased levels of FAP-1 in the test sample as compared to a non-cancer reference may indicate the presence of a cancer, or the presence of a more aggressive cancer. Decreased levels of CD95 or miR200 family members such as miR-200c as compared to a non-cancer reference may indicate the presence of a cancer, or the presence of a more aggressive cancer. The cMV population to be assessed can be isolated through immunoprecipitation, flow cytometry, or other isolation methodology disclosed herein or known in the art.

In a related aspect, the invention provides a method of characterizing a cancer comprising detecting a level of one or more biomarker, e.g., 1, 2, 3, 4, 5, 6, 7, 8, 9, 10, 11, 12, 13, 14, 15, 16, 17, 18, 19, 20, 21 or 22 biomarkers, selected from the group consisting of A2ML1, BAX, C10orf47, C1orf162, CSDA, EIFC3, ETFB, GABARAPL2, GUK1, GZMH, HIST1H3B, HLA-A, HSP90AA1, NRGN, PRDX5, PTMA, RABAC1, RABAGAP1L, RPL22, SAP18, SEPW 1, SOX1, and a combination thereof. The one or more biomarker can comprise PTMA (prothymosin, alpha), a member of the pro/parathymosin family which is cleaved into Thymosin alpha-1 and has a role in immune modulation. Thymosin alpha-1 is approved in at least 35 countries for the treatment of Hepatitis B and C, and it is also approved for inclusion with vaccines to boost the immune response in the treatment of other diseases. In an embodiment, the biomarkers comprise mRNA. The mRNAs can be isolated from vesicles that have been isolated as described herein. In some embodiments, a total vesicle population in a sample is isolated, e.g., by filtration or centrifugation. The vesicles can also by isolated by affinity, e.g., using a binding agent to a general vesicle biomarker, a disease biomarker or a cell-specific biomarker. The levels of the biomarkers can be compared to a control such as a sample without cancer, wherein a change between the levels of the biomarkers versus the control is used to characterize the cancer. The cancer can be a prostate cancer.

In an embodiment, the cancer assessed by the invention comprises prostate cancer and microRNAs (miRs) are used to differentiate between metastatic versus non-metastatic prostate cancer. Prostate cancer staging is a process of categorizing the risk of cancer spread beyond the prostate. Such spread is related to the probability of being cured with local therapies such as surgery or radiation. The information considered in such prognostic classification is based on clinical and pathological factors, including physical examination, imaging studies, blood tests and/or biopsy examination.

The most common scheme used to stage prostate cancer is promulgated by the American Joint Committee on Cancer, and is referred to as the TNM system. The TNM system evaluates the size of the tumor, the extent of involved lymph nodes, metastasis and also takes into account cancer grade. As with many other cancers, the cancers are often grouped by stage, e.g., stages I-IV). Generally, Stage I disease is cancer that is found incidentally in a small part of the sample when prostate tissue was removed for other reasons, such as benign prostatic hypertrophy, and the cells closely resemble normal cells and the gland feels normal to the examining finger. In Stage II more of the prostate is involved and a lump can be felt within the gland. In Stage III, the tumor has spread through the prostatic capsule and the lump can be felt on the surface of the gland. In Stage IV disease, the tumor has invaded nearby structures, or has spread to lymph nodes or other organs.

The Whitmore-Jewett stage is another staging scheme that is now used less often. The Gleason Grading System is based on cellular content and tissue architecture from biopsies, which provides an estimate of the destructive potential and ultimate prognosis of the disease.

The TNM tumor classification system can be used to describe the extent of cancer in a subject's body. T describes the size of the tumor and whether it has invaded nearby tissue, N describes regional lymph nodes that are involved, and M describes distant metastasis. TNM is maintained by the International Union Against Cancer (UICC) and is used by the American Joint Committee on Cancer (AJCC) and the International Federation of Gynecology and Obstetrics (FIGO). Those of skill in the art understand that not all tumors have TNM classifications such as, e.g., brain tumors. Generally, T (a,is,(0), 1-4) is measured as the size or direct extent of the primary tumor. N (0-3) refers to the degree of spread to regional lymph nodes: NO means that tumor cells are absent from regional lymph nodes, N1 means that tumor cells spread to the closest or small numbers of regional lymph nodes, N2 means that tumor cells spread to an extent between N1 and N3; N3 means that tumor cells spread to most distant or numerous regional lymph nodes. M (0/1) refers to the presence of metastasis: MX means that distant metastasis was not assessed; MO means that no distant metastasis are present; M1 means that metastasis has occurred to distant organs (beyond regional lymph nodes). M1 can be further delineated as follows: M1a indicates that the cancer has spread to lymph nodes beyond the regional ones; M1b indicates that the cancer has spread to bone; and M1c indicates that the cancer has spread to other sites (regardless of bone involvement). Other parameters may also be assessed. G (1-4) refers to the grade of cancer cells (i.e., they are low grade if they appear similar to normal cells, and high grade if they appear poorly differentiated). R (0/1/2) refers to the completeness of an operation (i.e., resection-boundaries free of cancer cells or not). L (0/1) refers to invasion into lymphatic vessels. V (0/1) refers to invasion into vein. C (1-4) refers to a modifier of the certainty (quality) of V.

Prostate tumors are often assessed using the Gleason scoring system. The Gleason scoring system is based on microscopic tumor patterns assessed by a pathologist while interpreting a biopsy specimen. When prostate cancer is present in the biopsy, the Gleason score is based upon the degree of loss of the normal glandular tissue architecture (i.e. shape, size and differentiation of the glands). The classic Gleason scoring system has five basic tissue patterns that are technically referred to as tumor “grades.” The microscopic determination of this loss of normal glandular structure caused by the cancer is represented by a grade, a number ranging from 1 to 5, with 5 being the worst grade. Grade 1 is typically where the cancerous prostate closely resembles normal prostate tissue. The glands are small, well-formed, and closely packed. At Grade 2 the tissue still has well-formed glands, but they are larger and have more tissue between them, whereas at Grade 3 the tissue still has recognizable glands, but the cells are darker. At high magnification, some of these cells in a Grade 3 sample have left the glands and are beginning to invade the surrounding tissue. Grade 4 samples have tissue with few recognizable glands and many cells are invading the surrounding tissue. For Grade 5 samples, the tissue does not have recognizable glands, and are often sheets of cells throughout the surrounding tissue.

miRs that distinguish metastatic and non-metastatic prostate cancer can be overexpressed in metastatic samples versus non-metastatic. Alternately, miRs that distinguish metastatic and non-metastatic prostate cancer can be overexpressed in non-metastatic samples versus metastatic. Useful miRs for distinguishing metastatic prostate cancer include one or more, e.g., 1, 2, 3, 4, 5, 6, 7 or 8, miRs selected from the group consisting of miR-495, miR-10a, miR-30a, miR-570, miR-32, miR-885-3p, miR-564, and miR-134. In another embodiment, miRs for distinguishing metastatic prostate cancer include one or more, e.g., 1, 2, 3, 4, 5, 6, 7, 8, 9, 10, 11, 12, 13 or 14, miRs selected from the group consisting of hsa-miR-375, hsa-miR-452, hsa-miR-200b, hsa-miR-146b-5p, hsa-miR-1296, hsa-miR-17*, hsa-miR-100, hsa-miR-574-3p, hsa-miR-20a*, hsa-miR-572, hsa-miR-1236, hsa-miR-181a, hsa-miR-937, and hsa-miR-23a*. In still another embodiment, useful miRs for distinguishing metastatic prostate cancer include, e.g., 1, 2, 3, 4, 5, 6, 7, 8 or 9, miRs selected from the group consisting of hsa-miR-200b, hsa-miR-375, hsa-miR-582-3p, hsa-miR-17*, hsa-miR-1296, hsa-miR-20a*, hsa-miR-100, hsa-miR-452, and hsa-miR-577. The miRs for distinguishing metastatic prostate cancer can be one or more, e.g., 1, 2, 3 or 4, miRs selected from the group consisting of miR-141, miR-375, miR-200b and miR-574-3p.

In an aspect, microRNAs (miRs) are used to differentiate between cancer and non-cancer samples. Vesicles derived from patient samples can be analyzed for miR payload contained within the vesicles. The sample can be a bodily fluid, including semen, urine, blood, serum or plasma. The sample can also comprise a tissue or biopsy sample. A number of different methodologies are available for detecting miRs. In some embodiments, arrays of miR panels are use to simultaneously query the expression of multiple miRs. The Exiqon mIRCURY LNA microRNA PCR system panel (Exiqon, Inc., Woburn, Mass.) can be used for such purposes. miRs that distinguish cancer can be overexpressed in cancer versus control samples. Alternately, miRs that distinguish cancer can be overexpressed in cancer samples versus controls. Useful miRs for distinguishing cancer from non-cancer include one or more, e.g., 1, 2, 3, 4, 5, 6, 7, 8, 9, 10, 11, 12 or 13, miRs selected from the group consisting of hsa-miR-574-3p, hsa-miR-331-3p, hsa-miR-326, hsa-miR-181a-2*, hsa-miR-130b, hsa-miR-301a, hsa-miR-141, hsa-miR-432, hsa-miR-107, hsa-miR-628-5p, hsa-miR-625*, hsa-miR-497, and hsa-miR-484. In another embodiment, useful miRs for distinguishing cancer from non-cancer include one or more, e.g., 1, 2, 3, 4, 5, 6, 7, 8, 9 or 10, miRs selected from the group consisting of hsa-miR-574-3p, hsa-miR-141, hsa-miR-331-3p, hsa-miR-432, hsa-miR-326, hsa-miR-2110, hsa-miR-107, hsa-miR-130b, hsa-miR-301a, and hsa-miR-625*. In still another embodiment, the useful miRs for distinguishing cancer from non-cancer include one or more, e.g., 1, 2, 3, 4, 5, 6, 7, 8 or 9, miRs selected from the group consisting of hsa-miR-107, hsa-miR-326, hsa-miR-432, hsa-miR-574-3p, hsa-miR-625*, hsa-miR-2110, hsa-miR-301a, hsa-miR-141 or hsa-miR-373*. The cancer can comprise those cancers listed above. In an exemplary embodiment, the cancer is a prostate cancer and the microRNAs (miRs) are used to differentiate between prostate cancer and non-cancer samples.

The method contemplates assessing combinations of circulating biomarkers. For example, multiple markers from antibody arrays and miR analysis can be used to distinguish prostate cancer from normal, BPH and PCa, or metastatic versus non-metastatic disease. In this manner, improved sensitivity, specificity, and/or accuracy can be obtained. In some embodiments, the levels of one or more, e.g., 1, 2, 3, 4, 5 or 6, miRs selected from the group consisting of hsa-miR-432, hsa-miR-143, hsa-miR-424, hsa-miR-204, hsa-miR-581f and hsa-miR-451 are detected in a patient sample to assess the presence of prostate cancer. Any of these miRs can be elevated in patients with PCa but having serum PSA<4.0 ng/ml. In an embodiment, the invention provides a method of assessing a prostate cancer, comprising determining a level of one or more, e.g., 1, 2, 3, 4, 5 or 6, miRs selected from the group consisting of hsa-miR-432, hsa-miR-143, hsa-miR-424, hsa-miR-204, hsa-miR-581f and hsa-miR-451 in a sample from a subject. The sample can be a bodily fluid, e.g., blood, plasma or serum. The miRs can be isolated in vesicles isolated from the sample. The subject can have a PSA level less than some threshold, such as 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, or 6.0 ng/ml in a blood sample. Higher levels of the miRs than in a reference sample can indicate the presence of PCa in the sample. In some embodiments, the reference comprises a level of the one or more miRs in control samples from subjects without PCa. In some embodiments, the reference comprises a level of the one or more miRs in control samples from subject with PCa and PSA levels≧some threshold, such as 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, or 6.0 ng/ml. The threshold can be 4.0 ng/ml.

In some embodiments of the invention, vesicles in patient samples are assessed to provide a diagnostic, prognostic or theranostic readout. Vesicle analysis of patient samples includes the detection of vesicle surface biomarkers, e.g., surface antigens, and/or vesicle payload, e.g., mRNAs and microRNAs, as described herein. Methods for analysis of vesicles are presented in PCT Patent Application PCT/US09/06095, entitled “METHODS AND SYSTEMS OF USING EXOSOMES FOR DETERMINING PHENOTYPES” and filed Nov. 12, 2009; U.S. Provisional Patent Application 61/362,674, entitled “METHODS AND SYSTEMS OF USING VESICLES FOR DETERMINING PHENOTYPES” and filed Jul. 7, 2010; and U.S. Provisional Patent Application 61/393,823, entitled “DETECTION OF GI CANCERS” and filed Oct. 15, 2010, which applications are incorporated by reference herein in their entirety.

In one aspect, the invention includes a method of identifying a bio-signature of one or more vesicles in a biological sample from said subject, wherein the bio-signature comprises analysis of vesicle surface antigens and vesicle payload. The surface antigens can comprise surface proteins and the vesicle payload can comprise microRNA. For example, vesicles can be captured using binding agents that recognize vesicle surface antigens, and the microRNA inside these captured vesicles can be assessed. Accordingly, the bio-signature may comprise the surface antigens used for capture as well as the microRNA inside the vesicles. The bio-signature can be used for diagnostic, prognostic or theranostic purposes. For example, the bio-signature can be a signature that identifies cancer, identifies aggressive or metastatic cancer, or identifies a cancer that is likely to respond to a candidate therapeutic agent.

As an illustrative example, consider a method of capturing vesicles in a sample using an antibody to B7H3 and then assessing the levels of miR-141 within the captured vesicles. In this example, the bio-signature comprises the level of miR-141 within exosomes displaying B7H3 on their surface. Depending on the levels of B7H3+ vesicles in the sample as well as the levels of miR-141 within the sample, the bio-signature may indicate that the sample comprises a cancer, comprises an aggressive cancer, is likely to respond to a certain treatment or chemotherapeutic agent, etc.

In one embodiment, the method of assessing cancer in a subject comprises: identifying a bio-signature of one or more vesicles in a biological sample from said subject, comprising: determining a level of one or more general vesicles protein biomarkers; determining a level of one or more cell-specific protein biomarkers; determining a level of one or more disease-specific protein biomarkers; and determining the level of one or more microRNA biomarkers in the vesicles, wherein said characterizing comprises comparing said levels of biomarkers in said sample to a reference to determine whether said subject may be predisposed to or afflicted with cancer. The protein biomarkers can be detected in a multiplex fashion in a single assay. The microRNA biomarkers can also be detected in a multiplex fashion in a single assay. In some cases, the cell-specific and disease-specific biomarker may overlap, e.g., one biomarker may serve to identify a cancer from a particular cellular origin. The biological sample can be a bodily fluid, such as blood, serum or plasma.

In an example, the method of the invention comprises a diagnostic test for prostate cancer comprising isolating vesicles from a blood sample from a patient to detect vesicles indicative of the presence or absence of prostate cancer. The blood can be serum or plasma. The vesicles are isolated by capture with “capture antibodies” that recognize specific vesicle surface antigens. The surface antigens for the prostate cancer diagnostic assay include the tetraspanins CD9, CD63 and CD81, which are generally present on vesicles in the blood and therefore act as general vesicle biomarkers, the prostate specific biomarkers PSMA and PCSA, and the cancer specific biomarker B7H3. In some cases, EpCam is used as a cancer specific biomarker as well or instead of B7H3. The capture antibodies can be tethered to a substrate. In an embodiment, the substrate comprises fluorescently labeled beads, wherein the beads are differentially labeled for each capture antibody. As desired, the payload of the detected vesicles can be assessed in order to characterize the cancer.

As described above, the biomarkers of the invention can be assessed to identify a biosignature. In an aspect, the invention provides a method comprising: determining a presence or level of one or more biomarker in a biological sample, wherein the one or more biomarker comprises one or more biomarker selected from Table 3, Table 4, and/or Table 5; and identifying a biosignature comprising the presence or level of the one or more biomarker. In some embodiments, the method further comprises comparing the biosignature to a reference biosignature, wherein the comparison is used to characterize a cancer, including the cancers disclosed herein or known in the art. The reference biosignature can be from a subject without the cancer. The reference biosignature can also be from the subject, e.g., from normal adjacent tissue or from a sample taken at another point in time. Various ways of characterizing a cancer are disclosed herein. For example, characterizing the cancer may comprise identifying the presence or risk of the cancer in a subject, or identifying the cancer in a subject as metastatic or aggressive. The comparing step comprises determining whether the biosignature is altered relative to the reference biosignature, thereby providing a prognostic, diagnostic or theranostic characterization for the cancer. The biological sample comprises a bodily fluid, including without limitation the bodily fluids disclosed herein. For example, the bodily fluid may comprise urine, blood or a blood derivative.

The one or more biomarker can be one or more biomarker, e.g., 1, 2, 3, 4, 5, 6, 7, 8, 9 or 10 or more, selected from the group consisting of miR-22, let7a, miR-141, miR-182, miR-663, miR-155, mirR-125a-5p, miR-548a-5p, miR-628-5p, miR-517*, miR-450a, miR-920, hsa-miR-619, miR-1913, miR-224*, miR-502-5p, miR-888, miR-376a, miR-542-5p, miR-30b*, miR-1179, and a combination thereof. In an embodiment, the one or more biomarker is selected from the group consisting of miR-22, let7a, miR-141, miR-920, miR-450a, and a combination thereof. The one or more biomarker, e.g., 1, 2, 3, 4, 5, 6, 7, 8, 9 or 10 or more, may be a messenger RNA (mRNA) selected from the group consisting of the genes in any of the Examples herein, and a combination thereof. For example, the one or more biomarker may comprise 1, 2, 3, 4, 5, 6, 7, 8, 9 or 10 or more messenger RNA (mRNA) selected from the group consisting of A2ML1, BAX, C10orf47, C1orf162, CSDA, EIFC3, ETFB, GABARAPL2, GUK1, GZMH, HIST1H3B, HLA-A, HSP90AA1, NRGN, PRDX5, PTMA, RABAC1, RABAGAP1L, RPL22, SAP18, SEPW 1, SOX1, and a combination thereof. The one or more biomarker may comprise 1, 2, 3, 4, 5, or 6 messenger RNA (mRNA) selected from the group consisting of A2ML1, GABARAPL2, PTMA, RABAC1, SOX1, EFTB, and a combination thereof. The one or more biomarker may be isolated as payload of a population of microvesicles. The population can be a total population of microvesicles from the sample or a specific population, such as a PCSA+ population. In an embodiment, the method is used to assess a prostate cancer. For example, the method can be used to distinguish a sample comprising prostate cancer from a sample without prostate cancer.

In an embodiment, the one or more biomarker, e.g., 1, 2, 3, 4, 5, 6, 7, 8, 9 or 10 or more biomarkers, is selected from the group consisting of CA-125, CA 19-9, c-reactive protein, CD95, FAP-1, EGFR, EGFRvIII, apolipoprotein AI, apolipoprotein CIII, myoglobin, tenascin C, MSH6, claudin-3, claudin-4, caveolin-1, coagulation factor III, CD9, CD36, CD37, CD53, CD63, CD81, CD136, CD147, Hsp70, Hsp90, Rab13, Desmocollin-1, EMP-2, CK7, CK20, GCDF15, CD82, Rab-5b, Annexin V, MFG-E8, HLA-DR, a miR200 microRNA, miR-200c, and a combination thereof. The one or more biomarker may comprise 1, 2, 3, 4 or 5 biomarker selected from the group consisting of CA-125, CA 19-9, c-reactive protein, CD95, FAP-1, and a combination thereof. The one or more biomarker may be isolated directly from sample, or as surface antigens or payload of a population of microvesicles. In an embodiment, the method is used to assess an ovarian cancer. For example, the method can be used to distinguish a sample comprising ovarian cancer from a sample without ovarian cancer. Alternately, the method can be used to distinguish amongst ovarian cancer having different stage or prognosis.

In another embodiment, the one or more biomarker, e.g., 1, 2, 3, 4, 5, 6, 7, 8, 9 or 10 or more biomarkers, is selected from the group consisting of hsa-miR-574-3p, hsa-miR-141, hsa-miR-432, hsa-miR-326, hsa-miR-2110, hsa-miR-181a-2*, hsa-miR-107, hsa-miR-301a, hsa-miR-484, hsa-miR-625*, and a combination thereof. The method can be used to assess a prostate cancer. For example, the method can be used to distinguish a sample comprising prostate cancer from a sample without prostate cancer. In still another embodiment, the one or more biomarker, e.g., 1, 2, 3, 4, 5, 6, 7, 8, 9 or 10 or more biomarkers, is selected from the group consisting of hsa-miR-582-3p, hsa-miR-20a*, hsa-miR-375, hsa-miR-200b, hsa-miR-379, hsa-miR-572, hsa-miR-513a-5p, hsa-miR-577, hsa-miR-23a*, hsa-miR-1236, hsa-miR-609, hsa-miR-17*, hsa-miR-130b, hsa-miR-619, hsa-miR-624*, hsa-miR-198, and a combination thereof. For example, the method can be used to distinguish a sample comprising metastatic prostate cancer from a sample with non-metastatic prostate cancer. The one or more biomarker may be isolated as payload of a population of microvesicles.

The one or more biomarker may be miR-497. The method can be used to assess a lung cancer. For example, the method can be used to distinguish a lung cancer sample from a non-cancer sample. The one or more biomarker may be isolated as payload of a population of microvesicles.

The one or more biomarker, e.g., 1, 2, 3, 4, 5, 6, 7, 8, 9 or 10 or more biomarkers, may comprise a messenger RNA (mRNA) selected from the group consisting of AQP2, BMP5, C16orf86, CXCL13, DST, ERCC1, GNAO1, KLHL5, MAP4K1, NELL2, PENK, PGF, POU3F1, PRSS21, SCML1, SEMG1, SMARCD3, SNA12, TAF1C, TNNT3, and a combination thereof. The mRNA may be isolated from microvesicles. The method can be used to characterize a prostate cancer, such as distinguish a prostate cancer sample from a normal sample without cancer. In another embodiment, the one or more biomarker, e.g., 1, 2, 3, 4, 5, 6, 7, 8, 9 or 10 or more biomarkers, comprises a messenger RNA (mRNA) selected from the group consisting of ADRB2, ARG2, C22orf32, CYorf14, EIF1AY, FEV, KLK2, KLK4, LRRC26, MAOA, NLGN4Y, PNPLA7, PVRL3, SIM2, SLC30A4, SLC45A3, STX19, TRIM36, TRPM8, and a combination thereof. The mRNA may be isolated from microvesicles. The method can be used to characterize a prostate cancer, such as distinguish a prostate cancer sample from a sample having another cancer, e.g., a breast cancer. In still another embodiment, the one or more biomarker, e.g., 1, 2, 3, 4, 5, 6, 7, 8, 9 or 10 or more biomarkers, comprises a messenger RNA (mRNA) selected from the group consisting of ADRB2, BAIAP2L2, C19orf33, CDX1, CEACAM6, EEF1A2, ERN2, FAM110B, FOXA2, KLK2, KLK4, LOC389816, LRRC26, MIPOL1, SLC45A3, SPDEF, TRIM31, TRIM36, ZNF613, and a combination thereof. The mRNA may be isolated from microvesicles. The method can be used to characterize a prostate cancer, such as distinguish a prostate cancer sample from a sample having another cancer, e.g., a colorectal cancer. In yet another embodiment, the one or more biomarker, e.g., 1, 2, 3, 4, 5, 6, 7, 8, 9 or 10 or more biomarkers, comprises a messenger RNA (mRNA) selected from the group consisting of ASTN2, CAB39L, CRIP1, FAM110B, FEV, GSTP1, KLK2, KLK4, LOC389816, LRRC26, MUC1, PNPLA7, SIM2, SLC45A3, SPDEF, TRIM36, TRPV6, ZNF613, and a combination thereof. The mRNA may be isolated from microvesicles. The method can be used to characterize a prostate cancer, such as distinguish a prostate cancer sample from a sample having another cancer, e.g., a lung cancer. The one or more biomarker can also be a microRNA that regulates one or more of the mRNAs used to characterize a prostate cancer. For example, the one or more biomarker, e.g., 1, 2, 3, 4, 5, 6, 7, 8, 9 or 10 or more biomarkers, may comprise a microRNA selected from the group consisting of miRs-26a+b, miR-15, miR-16, miR-195, miR-497, miR-424, miR-206, miR-342-5p, miR-186, miR-1271, miR-600, miR-216b, miR-519 family, miR-203, and a combination thereof. The microRNA can be assessed as payload of a microvesicle population.

In still another embodiment, the one or more biomarker, e.g., 1, 2, 3, 4, 5, 6, 7, 8, 9, 10, 12, 15, 20 or more biomarkers, is selected from the group consisting of A33, ABL2, ADAM10, AFP, ALA, ALIX, ALPL, ApoJ/CLU, ASCA, ASPH(A-10), ASPH(D01P), AURKB, B7H3, B7H4, BCNP, BDNF, CA125(MUC16), CA-19-9, C-Bir, CD10, CD151, CD24, CD41, CD44, CD46, CD59(MEM-43), CD63, CD66eCEA, CD81, CD9, CDA, CDADC1, CRMP-2, CRP, CXCL12, CXCR3, CYFRA21-1, DDX-1, DLL4, EGFR, Epcam, EphA2, ErbB2, ERG, EZH2, FASL, FLNA, FRT, GAL3, GATA2, GM-CSF, Gro-alpha, HAP, HER3(ErbB3), HSP70, HSPB1, hVEGFR2, iC3b, IL-1B, IL6R, IL6Unc, IL7Ralpha/CD127, IL8, INSIG-2, Integrin, KLK2, LAMN, Mammoglobin, M-CSF, MFG-E8, MIF, MISRII, MMP7, MMP9, MUC1, Muc1, MUC17, MUC2, Ncam, NDUFB7, NGAL, NK-2R(C-21), NT5E (CD73), p53, PBP, PCSA, PDGFRB, PIM1, PRL, PSA, PSMA, RAGE, RANK, RegIV, RUNX2, S100-A4, seprase/FAP, SERPINB3, SIM2(C-15), SPARC, SPC, SPDEF, SPP1, STEAP, STEAP4, TFF3, TGM2, TIMP-1, TMEM211, Trail-R2, Trail-R4, TrKB(poly), Trop2, Tsg101, TWEAK, UNC93A, VEGFA, wnt-5a(C-16), and a combination thereof. The one or more biomarker may be detected directly in a sample, or as surface antigens or payload of a population of microvesicles. In an embodiment, a binding agent to the one or more biomarker is used to capture a microvesicle population. The captured microvesicle population can be detected using another binding agent, e.g., a labeled binding agent to a general vesicle marker such as one or more protein in Table 3, or a cell-of-origin or a cancer-specific biomarker, e.g., a biomarker in Table 4 or 5. In an embodiment, the antigen used for detection comprises one or more of CD9, CD63, CD81, PCSA, MUC2, and MFG-E8. In an embodiment, the method is used to assess a prostate cancer. For example, the method can be used to distinguish a sample comprising prostate cancer from a sample without prostate cancer. Alternately, the method is used to distinguish amongst prostate cancers having different stage or prognosis.

In a related embodiment, the one or more biomarker, e.g., 1, 2, 3, 4, 5, 6, 7, 8, 9, 10, 12, 15, 20 or more biomarkers, is selected from the group consisting of A33, ADAM10, AMACR, ASPH (A-10), AURKB, B7H3, CA125, CA-19-9, C-Bir, CD24, CD3, CD41, CD63, CD66e CEA, CD81, CD9, CDADC1, CSA, CXCL12, DCRN, EGFR, EphA2, ERG, FLNA, FRT, GAL3, GM-CSF, Gro-alpha, HER 3 (ErbB3), hVEGFR2, IL6 Unc, Integrin, Mammaglobin, MFG-E8, MMP9, MUC1, MUC17, MUC2, NGAL, NK-2R(C-21), NY-ESO-1, PBP, PCSA, PIM1, PRL, PSA, PSIP1/LEDGF, PSMA, RANK, S100-A4, seprase/FAP, SIM2 (C-15), SPDEF, SSX2, STEAP, TGM2, TIMP-1, Trail-R4, Tsg 101, TWEAK, UNC93A, VCAN, XAGE-1, and a combination thereof. The one or more biomarker may be detected directly in a sample, or as surface antigens or payload of a population of microvesicles. In an embodiment, a binding agent to the one or more biomarker is used to capture a microvesicle population. The captured microvesicle population can be detected using another binding agent, e.g., a labeled binding agent to a general vesicle marker such as one or more protein in Table 3, or a cell-of-origin or or cancer-specific biomarker, e.g., a biomarker in Table 4 or 5. In an embodiment, the antigen used for detection comprises one or more of EpCAM, CD81, PCSA, MUC2 and MFG-E8. In an embodiment, the method is used to assess a prostate cancer. For example, the method can be used to distinguish a sample comprising prostate cancer from a sample without prostate cancer. Alternately, the method is used to distinguish amongst prostate cancers having different stage or prognosis.

In another related embodiment, the one or more biomarker, e.g., 1, 2, 3, 4, 5, 6, 7, 8, 9, 10, 12, 15, 20 or more biomarkers, is selected from the group consisting of A33, ADAM10, ALIX, AMACR, ASCA, ASPH (A-10), AURKB, B7H3, BCNP, CA125, CA-19-9, C-Bir (Flagellin), CD24, CD3, CD41, CD63, CD66e CEA, CD81, CD9, CDADC1, CRP, CSA, CXCL12, CYFRA21-1, DCRN, EGFR, EpCAM, EphA2, ERG, FLNA, GAL3, GATA2, GM-CSF, Gro alpha, HER3 (ErbB3), HSP70, hVEGFR2, iC3b, IL-1B, IL6 Unc, IL8, Integrin, KLK2, Mammaglobin, MFG-E8, MMP7, MMP9, MS4A1, MUC1, MUC17, MUC2, NGAL, NK-2R(C-21), NY-ESO-1, p53, PBP, PCSA, PIM1, PRL, PSA, PSMA, RANK, RUNX2, S100-A4, seprase/FAP, SERPINB3, SIM2 (C-15), SPC, SPDEF, SSX2, SSX4, STEAP, TGM2, TIMP-1, TRAIL R2, Trail-R4, Tsg 101, TWEAK, VCAN, VEGF A, XAGE, and a combination thereof. The one or more biomarker may be detected directly in a sample, or as surface antigens or payload of a population of microvesicles. In an embodiment, a binding agent to the one or more biomarker is used to capture a microvesicle population. The captured microvesicle population can be detected using another binding agent, e.g., a labeled binding agent to a general vesicle marker such as one or more protein in Table 3, or a cell-of-origin or or cancer-specific biomarker, e.g., a biomarker in Table 4 or 5. In an embodiment, the antigen used for detection comprises one or more of EpCAM, CD81, PCSA, MUC2 and MFG-E8. In an embodiment, the method is used to assess a prostate cancer. For example, the method can be used to distinguish a sample comprising prostate cancer from a sample without prostate cancer. Alternately, the method is used to distinguish amongst prostate cancers having different stage or prognosis.

In still another related embodiment, the one or more biomarker, e.g., 1, 2, 3, 4, 5, 6, 7, 8, 9, 10, 11, 12, 13, 14 or 15 biomarkers, is selected from the group consisting of ADAM-10, BCNP, CD9, EGFR, EpCam, IL1B, KLK2, MMP7, p53, PBP, PCSA, SERPINB3, SPDEF, SSX2, SSX4, and a combination thereof. The one or more biomarker may be detected directly in a sample, or as surface antigens or payload of a population of microvesicles. In an embodiment, a binding agent to the one or more biomarker is used to capture a microvesicle population. The captured microvesicle population can be detected using another binding agent, e.g., a labeled binding agent to a general vesicle marker such as one or more protein in Table 3, or a cell-of-origin or or cancer-specific biomarker, e.g., a biomarker in Table 4 or 5. In an embodiment, the antigen used for detection comprises one or more of EpCAM, KLK2, PBP, SPDEF, SSX2, SSX4. In a non-limiting example, consider that the detector binding agent is a binding agent to EpCam, e.g., an antibody or aptamer to EpCam, wherein the antibody or aptamer is optionally labeled to facilitate detection thereof. In such case, the one or more biomarker comprises one or more pair of biomarkers selected from the group consisting of EpCam-ADAM-10, EpCam-BCNP, EpCam-CD9, EpCam-EGFR, EpCam-EpCam, EpCam-IL1B, EpCam-KLK2, EpCam-MMP7, EpCam-p53, EpCam-PBP, EpCam-PCSA, EpCam-SERPINB3, EpCam-SPDEF, EpCam-SSX2, EpCam-SSX4, and a combination thereof. In an embodiment, the method is used to assess a prostate cancer. For example, the method can be used to distinguish a sample comprising prostate cancer from a sample without prostate cancer. Alternately, the method is used to distinguish amongst prostate cancers having different stage or prognosis.

In one embodiment, the one or more biomarker, e.g., 1, 2, 3, 4, 5, 6, 7, 8, 9 or 10 or more biomarkers, is selected from the group consisting of miR-148a, miR-329, miR-9, miR-378*, miR-25, miR-614, miR-518c*, miR-378, miR-765, let-7f-2*, miR-574-3p, miR-497, miR-32, miR-379, miR-520g, miR-542-5p, miR-342-3p, miR-1206, miR-663, miR-222, and a combination thereof. In another embodiment, the one or more biomarker, e.g., 1, 2, 3, 4, 5, 6, 7, 8, 9 or 10 or more biomarkers, is selected from the group consisting of hsa-miR-877*, hsa-miR-593, hsa-miR-595, hsa-miR-300, hsa-miR-324-5p, hsa-miR-548a-5p, hsa-miR-329, hsa-miR-550, hsa-miR-886-5p, hsa-miR-603, hsa-miR-490-3p, hsa-miR-938, hsa-miR-149, hsa-miR-150, hsa-miR-1296, hsa-miR-384, hsa-miR-487a, hsa-miRPlus-C1089, hsa-miR-485-3p, hsa-miR-525-5p, and a combination thereof. The method can be used to assess a prostate cancer. For example, the method can be used to distinguish a sample comprising prostate cancer from a sample without prostate cancer. In still another embodiment, the one or more biomarker, e.g., 1, 2, 3, 4, 5, 6, 7, 8, 9 or 10 or more biomarkers, is selected from the group consisting of miR-588, miR-1258, miR-16-2*, miR-938, miR-526b, miR-92b*, let-7d, miR-378*, miR-124, miR-376c, miR-26b, miR-1204, miR-574-3p, miR-195, miR-499-3p, miR-2110, miR-888, and a combination thereof. For example, the method can be used to distinguish a sample comprising prostate cancer from a sample with inflammatory prostate disease. The one or more biomarker may be isolated as payload of a population of microvesicles.

In one embodiment, the one or more biomarker, e.g., 1, 2, 3, 4, 5, 6, 7, 8, 9 or 10 or more biomarkers, is selected from the group consisting of let-7d, miR-148a, miR-195, miR-25, miR-26b, miR-329, miR-376c, miR-574-3p, miR-888, miR-9, miR1204, miR-16-2*, miR-497, miR-588, miR-614, miR-765, miR92b*, miR-938, let-7f-2*, miR-300, miR-523, miR-525-5p, miR-1182, miR-1244, miR-520d-3p, miR-379, let-7b, miR-125a-3p, miR-1296, miR-134, miR-149, miR-150, miR-187, miR-32, miR-324-3p, miR-324-5p, miR-342-3p, miR-378, miR-378*, miR-384, miR-451, miR-455-3p, miR-485-3p, miR-487a, miR-490-3p, miR-502-5p, miR-548a-5p, miR-550, miR-562, miR-593, miR-593*, miR-595, miR-602, miR-603, miR-654-5p, miR-877*, miR-886-5p, miR-125a-5p, miR-140-3p, miR-192, miR-196a, miR-2110, miR-212, miR-222, miR-224*, miR-30b*, miR-499-3p, miR-505*, and a combination thereof. In another embodiment, the one or more biomarker, e.g., 1, 2, 3, 4, 5, 6, 7, 8, 9 or 10 or more biomarkers, is selected from the group consisting of hsa-miR-451, hsa-miR-223, hsa-miR-593*, hsa-miR-1974, hsa-miR-486-5p, hsa-miR-19b, hsa-miR-320b, hsa-miR-92a, hsa-miR-21, hsa-miR-675*, hsa-miR-16, hsa-miR-876-5p, hsa-miR-144, hsa-miR-126, hsa-miR-137, hsa-miR-1913, hsa-miR-29b-1*, hsa-miR-15a, hsa-miR-93, hsa-miR-1266, and a combination thereof. The method can be used to assess a prostate cancer. For example, the method can be used to distinguish a sample comprising prostate cancer from a sample without prostate cancer. The one or more biomarker may be isolated as payload of a population of microvesicles. The population can comprise PCSA+ microvesicles. In an embodiment, the population consists of PCSA+ microvesicles. In one embodiment, a population of PCSA+ vesicles is isolated and microRNA within the isolated vesicles are assessed using methods as described herein or known in the art. Elevated levels of miR-1974 in a test sample as compared to a control sample (e.g., non-cancer sample) are indicative of a prostate cancer in the test sample. Similarly, decreased levels of miR-320b in a test sample as compared to a control sample (e.g., non-cancer sample) can indicate the presence of a prostate cancer in the test sample.

The one or more biomarker can comprise EpCAM and MMP7. The biomarkers may be isolated from microvesicles. In an embodiment, EpCAM+/MMP7+ microvesicles are detected in a sample, such as blood or a blood derivative. In a non-limiting example, the EpCAM+/MMP7+ microvesicles are identified by EpCAM and MMP7 binding agents using methods as described herein, e.g., using flow cytometry. As described, vesicles in a biological sample can be identified by flow sorting using general vesicle markers, e.g., the marker in Table 3 such as tetraspanins including CD9, CD63 and/or CD81. The levels of the EpCAM+/MMP7+ microvesicles can be used to characterize a cancer, such as distinguish a cancer sample from a normal sample without cancer. In one embodiment, lower levels of MMP7 in EpCAM+ vesicles as compared to a non-cancer control sample indicate the presense of cancer. As EpCAM and MMP7 comprise cancer markers, one of skill will appreciate that the method can be used to assess various cancers in a sample. In an embodiment, the cancer comprises prostate cancer.

In another embodiment, the one or more biomarker comprises a transcription factor. The transcription factor can be one or more, e.g., 2, 3, 4, 5, 6, 7, 8, 9 or 10 of c-Myc, AEBP1, HNF4a, STAT3, EZH2, p53, MACC1, SPDEF, RUNX2 and YB-1. In another embodiment, the one or more biomarker may also comprise a kinase. The kinase can be one or more of AURKA and AURKB. The method can be used to assess a prostate cancer. For example, the method can be used to distinguish a sample comprising prostate cancer from a sample without prostate cancer. The one or more biomarker may be isolated as payload of a population of microvesicles. In an embodiment, elevated levels of the transcription factors and/or kinases in the microvesicle population as compared to normal controls indicate the presence of a cancer. As these are cancer-related transcription factors, one of skill will appreciate that any appropriate cancer can be assessed using the method. In an embodiment, the cancer comprises a prostate cancer or a breast cancer.

The one or more biomarker can comprise PCSA, Muc2 and Adam10. The biomarkers may be isolated from microvesicles. In an embodiment, PCSA+/Muc2+/Adam10+ microvesicles are detected in a sample, such as blood or a blood derivative. In a non-limiting example, the PCSA+/Muc2+/Adam10+ microvesicles are identified by PCSA, Muc2 and Adam10 binding agents using methods as described herein, e.g., using flow cytometry. As described, vesicles in a biological sample can be identified by flow sorting using general vesicle markers, e.g., the marker in Table 3 such as tetraspanins including CD9, CD63 and/or CD81. The levels of the PCSA+/Muc2+/Adam10+ microvesicles can be used to characterize a cancer, such as distinguish a cancer sample from a normal sample without cancer. In one embodiment, elevated levels of PCSA+/Muc2+/Adam10+ vesicles as compared to a non-cancer control sample indicate the presense of cancer. In an embodiment, the cancer comprises prostate cancer.

In one embodiment, the one or more biomarker, e.g., 1, 2, 3, 4, 5, 6, 7, 8, 9 or 10 or more biomarkers, is selected from the group consisting Alkaline Phosphatase (AP), CD63, MyoD1, Neuron Specific Enolase, MAP1B, CNPase, Prohibitin, CD45RO, Heat Shock Protein 27, Collagen II, Laminin B1/b1, Gail, CDw75, bcl-XL, Laminin-s, Ferritin, CD21, ADP-ribosylation Factor (ARF-6). In another embodiment, the one or more biomarker, e.g., 1, 2, 3, 4, 5, 6, 7, 8, 9 or 10 or more biomarkers, is selected from the group consisting of CD56/NCAM-1, Heat Shock Protein 27/hsp27, CD45RO, MAP1B, MyoD1, CD45/T200/LCA, CD3zeta, Laminin-s, bcl-XL, Rad18, Gail, Thymidylate Synthase, Alkaline Phosphatase (AP), CD63, MMP-16/MT3-MMP, Cyclin C, Neuron Specific Enolase, SIRP al, Laminin B1/b1, Amyloid Beta (APP), SODD (Silencer of Death Domain), CDC37, Gab-1, E2F-2, CD6, Mast Cell Chymase, Gamma Glutamylcysteine Synthetase (GCS), and a combination thereof. The one or more biomarker can comprise protein. The one or more biomarker may be isolated as payload of a population of microvesicles. The method can be used to assess a prostate cancer. For example, the method can be used to distinguish a sample comprising prostate cancer from a control sample without prostate cancer. The control sample can be a sample from a non-diseased state, a non-malignant prostate condition, or it can be a sample indicative of another type of cancer or related disorder, such as a breast cancer, brain cancer, lung cancer, colorectal cancer or colorectal adenoma. In an embodiment, elevated levels of Alkaline Phosphatase (AP) as compared to the control indicate the presence of prostate cancer. Similarly, elevated levels of CD56 (NCAM) as compared to the control can indicate the presence of prostate cancer. In an embodiment, elevated levels of CD-3 zeta as compared to the control indicate the presence of prostate cancer. In anther embodiment, elevated levels of Map1b as compared to the control can indicate the presence of prostate cancer. Conversely, elevated levels of 14.3.3 and/or filamin may indicate a colorectal cancer and not prostate cancer or other cancers or prostate disorders. Similarly, elevated levels of thrombospondin may indicate a colorectal or lung cancer and not prostate cancer or other cancers or prostate disorders.

In one embodiment, the one or more biomarker comprises MMP7. The one or more biomarker can comprise protein. The one or more biomarker may be a surface antigen or payload of a population of microvesicles. The method can be used to assess a cancer. One of skill will appreciate that any appropriate cancer can be assessed using the method as MMP7 is a known cancer marker. In an embodiment, the cancer comprises a prostate cancer.

In some embodiments, the one or more biomarker comprises a protein selected from the group consisting of A33, ABL2, ADAM10, AFP, ALA, ALIX, ALPL, ApoJ/CLU, ASCA, ASPH(A-10), ASPH(D01P), AURKB, B7H3, B7H3, B7H4, BCNP, BDNF, CA125(MUC16), CA-19-9, C-Bir, CD10, CD151, CD24, CD41, CD44, CD46, CD59(MEM-43), CD63, CD66eCEA, CD81, CD9, CDA, CDADC1, CRMP-2, CRP, CXCL12, CXCR3, CYFRA21-1, DDX-1, DLL4, EGFR, Epcam, EphA2, ErbB2, ERG, EZH2, FASL, FLNA, FRT, GAL3, GATA2, GM-CSF, Gro-alpha, HAP, HER3(ErbB3), HSP70, HSPB1, hVEGFR2, iC3b, IL-1B, IL6R, IL6Unc, IL7Ralpha/CD127, IL8, INSIG-2, Integrin, KLK2, LAMN, Mammoglobin, M-CSF, MFG-E8, MIF, MISRII, MMP7, MMP9, MUC1, MUC17, MUC2, Ncam, NDUFB7, NGAL, NK-2R(C-21), NT5E (CD73), p53, PBP, PCSA, PDGFRB, PIM1, PRL, PSA, PSMA, RAGE, RANK, RegIV, RUNX2, S100-A4, seprase/FAP, SERPINB3, SIM2(C-15), SPARC, SPC, SPDEF, SPP1, STEAP, STEAP4, TFF3, TGM2, TIMP-1, TMEM211, Trail-R2, Trail-R4, TrKB(poly), Trop2, Tsg101, TWEAK, UNC93A, VEGFA, wnt-5a(C-16), and a combination thereof. The one or more biomarker may further comprise a protein selected from the group consisting of CD9, CD63, CD81, PCSA, MUC2, MFG-E8, and a combination thereof. In some embodiments, the biosignature is used to characterize a cancer, e.g., a prostate cancer.

In still other embodiments, the one or more biomarker comprises a protein selected from the group consisting of A33, ADAM10, AMACR, ASPH (A-10), AURKB, B7H3, CA125, CA-19-9, C-Bir, CD24, CD3, CD41, CD63, CD66e CEA, CD81, CD9, CDADC1, CSA, CXCL12, DCRN, EGFR, EphA2, ERG, FLNA, FRT, GAL3, GM-CSF, Gro-alpha, HER 3 (ErbB3), hVEGFR2, IL6 Unc, Integrin, Mammaglobin, MFG-E8, MMP9, MUC1, MUC17, MUC2, NGAL, NK-2R(C-21), NY-ESO-1, PBP, PCSA, PIM1, PRL, PSA, PSIP1/LEDGF, PSMA, RANK, S100-A4, seprase/FAP, SIM2 (C-15), SPDEF, SSX2, STEAP, TGM2, TIMP-1, Trail-R4, Tsg 101, TWEAK, UNC93A, VCAN, XAGE-1, and a combination thereof. The one or more biomarker may further comprise a protein selected from the group consisting of EpCAM, CD81, PCSA, MUC2, MFG-E8, and a combination thereof. In some embodiments, the biosignature is used to characterize a prostate cancer.

In still other embodiments, the one or more biomarker comprises a protein selected from the group consisting of the one or more biomarker comprises a protein selected from the group consisting of A33, ADAM10, ALIX, AMACR, ASCA, ASPH (A-10), AURKB, B7H3, BCNP, CA125, CA-19-9, C-Bir (Flagellin), CD24, CD3, CD41, CD63, CD66e CEA, CD81, CD9, CDADC1, CRP, CSA, CXCL12, CYFRA21-1, DCRN, EGFR, EpCAM, EphA2, ERG, FLNA, GAL3, GATA2, GM-CSF, Gro alpha, HER3 (ErbB3), HSP70, hVEGFR2, iC3b, IL-1B, IL6 Unc, IL8, Integrin, KLK2, Mammaglobin, MFG-E8, MMP7, MMP9, MS4A1, MUC1, MUC17, MUC2, NGAL, NK-2R(C-21), NY-ESO-1, p53, PBP, PCSA, PIM1, PRL, PSA, PSMA, RANK, RUNX2, S100-A4, seprase/FAP, SERPINB3, SIM2 (C-15), SPC, SPDEF, SSX2, SSX4, STEAP, TGM2, TIMP-1, TRAIL R2, Trail-R4, Tsg 101, TWEAK, VCAN, VEGF A, XAGE, and a combination thereof. The one or more biomarker may further comprise a protein selected from the group consisting of EpCAM, CD81, PCSA, MUC2, MFG-E8, and a combination thereof. In some embodiments, the biosignature is used to characterize a cancer, e.g., a prostate cancer.

In an embodiment, the one or more biomarker comprises one or more protein selected from the group consisting of CD9, CD63, CD81, MMP7, EpCAM, and a combination thereof. The one or more biomarker can be a protein selected from the group consisting of STAT3, EZH2, p53, MACC1, SPDEF, RUNX2, YB-1, AURKA, AURKB, and a combination thereof. The one or more biomarker can be a protein selected from the group consisting of PCSA, Muc2, Adam10, and a combination thereof. The one or more biomarker can include MMP7. The biosignature can be used to detect a cancer, e.g., a breast or prostate cancer.

In another embodiment, the one or more biomarker comprises a protein selected from the group consisting of Alkaline Phosphatase (AP), CD63, MyoD1, Neuron Specific Enolase, MAP1B, CNPase, Prohibitin, CD45RO, Heat Shock Protein 27, Collagen II, Laminin B1/b1, Gail, CDw75, bcl-XL, Laminin-s, Ferritin, CD21, ADP-ribosylation Factor (ARF-6), and a combination thereof. The one or more biomarker may comprise a protein selected from the group consisting of CD56/NCAM-1, Heat Shock Protein 27/hsp27, CD45RO, MAP1B, MyoD1, CD45/T200/LCA, CD3zeta, Laminin-s, bcl-XL, Rad18, Gail, Thymidylate Synthase, Alkaline Phosphatase (AP), CD63, MMP-16/MT3-MMP, Cyclin C, Neuron Specific Enolase, SIRP al, Laminin B1/b1, Amyloid Beta (APP), SODD (Silencer of Death Domain), CDC37, Gab-1, E2F-2, CD6, Mast Cell Chymase, Gamma Glutamylcysteine Synthetase (GCS), and a combination thereof. For example, the one or more biomarker may comprise a protein selected from the group consisting of Alkaline Phosphatase (AP), CD56 (NCAM), CD-3 zeta, Map1b, 14.3.3 pan, filamin, thrombospondin, and a combination thereof. The biosignature can be used to characterize a cancer. For example, the biosignature may be used to distinguish between a prostate cancer and other prostate disorders. The biosignature may also be used to distinguish between a prostate cancer and other cancers, e.g., lung, colorectal, breast and brain cancer.

In another embodiment, the one or more biomarker comprises a protein selected from the group consisting of ADAM-10, BCNP, CD9, EGFR, EpCam, IL1B, KLK2, MMP7, p53, PBP, PCSA, SERPINB3, SPDEF, SSX2, SSX4, and a combination thereof. For example, the one or more biomarker may comprise a protein selected from the group consisting of EGFR, EpCAM, KLK2, PBP, SPDEF, SSX2, SSX4, and a combination thereof. The one or more biomarker may also comprise a protein selected from the group consisting of EpCAM, KLK2, PBP, SPDEF, SSX2, SSX4, and a combination thereof.

In some embodiments, combinations of biomarkers are detected. For example, the method of the invention may comprise use of a first reagent and a second reagent that specifically bind to one or more microvesicle-associated biomarker disclosed herein, e.g., in any of Table 3, Table 4 and/or Table 5. The method may further comprise comparing the biosignature to a reference biosignature, wherein the comparison is used to characterize a cancer. The reference biosignature can be from a subject without the cancer. The reference biosignature can be from the subject. For example, the reference biosignature can be from a non-malignant sample from the subject such as normal adjacent tissue, or a different sample taken from the subject over a time course. The characterizing may comprise identifying the presence or risk of the cancer in a subject, or identifying the cancer in a subject as metastatic or aggressive. The comparing step may comprise determining whether the biosignature is altered relative to the reference biosignature, thereby providing a prognostic, diagnostic or theranostic determination for the cancer.

In an embodiment, the first reagent comprises a capture agent and the second reagent comprises a detector agent. The first and second reagents may comprise antibodies, aptamers, or a combination thereof. In an embodiment, the capture agent is tethered to a substrate, e.g., a well of a microtiter plate, a planar array, a microbead, a column packing material, or the like. The detector agent may be labeled to facilitate its detection. The label may be a fluorescent label, radiolabel, enzymatic label, or the like. The detector agent may be labeled directly or indirectly. Techniques for capture and detection are further described herein.

The capture and detector agents can be chosen to recognize any useful pairs of biomarkers disclosed herein. For example, the capture and detector agents can be selected from one or more pair of capture and detector agents in any of Tables 28-40 and 44-46. The invention also contemplates use of multiple pairs of capture and detector agents. In an embodiment, the one or more pair of capture and detector agents comprises binding agent pairs to Mammaglobin-MFG-E8, SIM2-MFG-E8 and NK-2R-MFG-E8. In another embodiment, the one or more pair of capture and detector agents comprises binding agent pairs to Integrin-MFG-E8, NK-2R-MFG-E8 and Gal3-MFG-E8. In still another embodiment, the one or more pair of capture and detector agents comprises capture agents to AURKB, A33, CD63, Gro-alpha, and Integrin; and detector agents to MUC2, PCSA, and CD81. The one or more pair of capture and detector agents may also comprise capture agents to AURKB, CD63, FLNA, A33, Gro-alpha, Integrin, CD24, SSX2, and SIM2; and detector agents to MUC2, PCSA, CD81, MFG-E8, and EpCam. The one or more pair of capture and detector agents can comprise binding agent pairs to EpCam-MMP7, PCSA-MMP7, and EpCam-BCNP. In an embodiment, the one or more pair of capture and detector agents comprises binding agent pairs to EpCam-MMP7, PCSA-MMP7, EpCam-BCNP, PCSA-ADAM10, and PCSA-KLK2. In another embodiment, the one or more pair of capture and detector agents comprises binding agent pairs to EpCam-MMP7, PCSA-MMP7, EpCam-BCNP, PCSA-ADAM10, PCSA-KLK2, PCSA-SPDEF, CD81-MMP7, PCSA-EpCam, MFGE8-MMP7 and PCSA-IL-8. In still another embodiment, the one or more pair of capture and detector agents comprises binding agent pairs to EpCam-MMP7, PCSA-MMP7, EpCam-BCNP, PCSA-ADAM10, and CD81-MMP7. Unless otherwise specified, the binding agent pairs disclosed herein may comprise both “target of capture agent”-“target of detector agent” and “target of detector agent”-“target of capture agent.”

In one embodiment, the one or more pair of capture and detector agents comprises capture agents to one or more of ADAM-10, BCNP, CD9, EGFR, EpCam, IL1B, KLK2, MMP7, p53, PBP, PCSA, SERPINB3, SPDEF, SSX2, and SSX4. The pairs may further comprise a detector agent to EpCam. The pairs may also comprise a detector agent to PCSA. The biosignature can be used to characterize a prostate cancer, such as to detect microvesicles shed from prostate cancer cells, to distinguish a prostate cancer from a non-cancer sample, to stage or grade the cancer, or to provide a diagnosis, prognosis or theranosis.

In another embodiment, the one or more pair of capture and detector agents comprises binding agent pairs selected from the group consisting of EpCAM-EpCAM, EpCAM-KLK2, EpCAM-PBP, EpCAM-SPDEF, EpCAM-SSX2, EpCAM-SSX4, EpCAM-ADAM-10, EpCAM-SERPINB3, EpCAM-PCSA, EpCAM-p53, EpCAM-MMP7, EpCAM-IL1B, EpCAM-EGFR, EpCAM-CD9, EpCAM-BCNP, KLK2-EpCAM, KLK2-KLK2, KLK2-PBP, KLK2-SPDEF, KLK2-SSX2, KLK2-SSX4, KLK2-ADAM-10, KLK2-SERPINB3, KLK2-PCSA, KLK2-p53, KLK2-MMP7, KLK2-IL1B, KLK2-EGFR, KLK2-CD9, KLK2-BCNP, PBP-EpCAM, PBP-KLK2, PBP-PBP, PBP-SPDEF, PBP-SSX2, PBP-SSX4, PBP-ADAM-10, PBP-SERPINB3, PBP-PCSA, PBP-p53, PBP-MMP7, PBP-IL1B, PBP-EGFR, PBP-CD9, PBP-BCNP, SPDEF-EpCAM, SPDEF-KLK2, SPDEF-PBP, SPDEF-SPDEF, SPDEF-SSX2, SPDEF-SSX4, SPDEF-ADAM-10, SPDEF-SERPINB3, SPDEF-PCSA, SPDEF-p53, SPDEF-MMP7, SPDEF-IL1B, SPDEF-EGFR, SPDEF-CD9, SPDEF-BCNP, SSX2-EpCAM, SSX2-KLK2, SSX2-PBP, SSX2-SPDEF, SSX2-SSX2, SSX2-SSX4, SSX2-ADAM-10, SSX2-SERPINB3, SSX2-PCSA, SSX2-p53, SSX2-MMP7, SSX2-IL1B, SSX2-EGFR, SSX2-CD9, SSX2-BCNP, SSX4-EpCAM, SSX4-KLK2, SSX4-PBP, SSX4-SPDEF, SSX4-SSX2, SSX4-SSX4, SSX4-ADAM-10, SSX4-SERPINB3, SSX4-PCSA, SSX4-p53, SSX4-MMP7, SSX4-IL1B, SSX4-EGFR, SSX4-CD9, SSX4-BCNP, ADAM-10-EpCAM, ADAM-10-KLK2, ADAM-10-PBP, ADAM-10-SPDEF, ADAM-10-SSX2, ADAM-10-SSX4, ADAM-10-ADAM-10, ADAM-10-SERPINB3, ADAM-10-PCSA, ADAM-10-p53, ADAM-10-MMP7, ADAM-10-IL1B, ADAM-10-EGFR, ADAM-10-CD9, ADAM-10-BCNP, SERPINB3-EpCAM, SERPINB3-KLK2, SERPINB3-PBP, SERPINB3-SPDEF, SERPINB3-SSX2, SERPINB3-SSX4, SERPINB3-ADAM-10, SERPINB3-SERPINB3, SERPINB3-PCSA, SERPINB3-p53, SERPINB3-MMP7, SERPINB3-IL1B, SERPINB3-EGFR, SERPINB3-CD9, SERPINB3-BCNP, PCSA-EpCAM, PCSA-KLK2, PCSA-PBP, PCSA-SPDEF, PCSA-SSX2, PCSA-SSX4, PCSA-ADAM-10, PCSA-SERPINB3, PCSA-PCSA, PCSA-p53, PCSA-MMP7, PCSA-IL1B, PCSA-EGFR, PCSA-CD9, PCSA-BCNP, p53-EpCAM, p53-KLK2, p53-PBP, p53-SPDEF, p53-SSX2, p53-SSX4, p53-ADAM-10, p53-SERPINB3, p53-PCSA, p53-p53, p53-MMP7, p53-IL1B, p53-EGFR, p53-CD9, p53-BCNP, MMP7-EpCAM, MMP7-KLK2, MMP7-PBP, MMP7-SPDEF, MMP7-SSX2, MMP7-SSX4, MMP7-ADAM-10, MMP7-SERPINB3, MMP7-PCSA, MMP7-p53, MMP7-MMP7, MMP7-IL1B, MMP7-EGFR, MMP7-CD9, MMP7-BCNP, IL1B-EpCAM, IL1B-KLK2, IL1B-PBP, IL1B-SPDEF, IL1B-SSX2, IL1B-SSX4, IL1B-ADAM-10, IL1B-SERPINB3, IL1B-PCSA, IL1B-p53, IL1B-MMP7, IL1B-IL1B, IL1B-EGFR, IL1B-CD9, IL1B-BCNP, EGFR-EpCAM, EGFR-KLK2, EGFR-PBP, EGFR-SPDEF, EGFR-SSX2, EGFR-SSX4, EGFR-ADAM-10, EGFR-SERPINB3, EGFR-PCSA, EGFR-p53, EGFR-MMP7, EGFR-IL1B, EGFR-EGFR, EGFR-CD9, EGFR-BCNP, CD9-EpCAM, CD9-KLK2, CD9-PBP, CD9-SPDEF, CD9-SSX2, CD9-SSX4, CD9-ADAM-10, CD9-SERPINB3, CD9-PCSA, CD9-p53, CD9-MMP7, CD9-IL1B, CD9-EGFR, CD9-CD9, CD9-BCNP, BCNP-EpCAM, BCNP-KLK2, BCNP-PBP, BCNP-SPDEF, BCNP-SSX2, BCNP-SSX4, BCNP-ADAM-10, BCNP-SERPINB3, BCNP-PCSA, BCNP-p53, BCNP-MMP7, BCNP-IL1B, BCNP-EGFR, BCNP-CD9, BCNP-BCNP, and a combination thereof. As listed in this paragraph, the pairs comprise “target of capture agent”-“target of detector agent.” The biosignature can be used to characterize a prostate cancer.

In an embodiment, the one or more pair of capture and detector agents comprises capture agents to one or more of EpCAM, KLK2, PBP, SPDEF, SSX2, SSX4, EGFR; and a detector agent to EpCam. The biosignature can be used to characterize a prostate cancer.

As noted, the one or more microvesicle may be detected using multiple pairs of capture and detector agents. In an embodiment, the one or more pair of capture and detector agents comprises a plurality of capture agents selected from the group consisting of SSX4 and EpCAM; SSX4 and KLK2; SSX4 and PBP; SSX4 and SPDEF; SSX4 and SSX2; SSX4 and EGFR; SSX4 and MMP7; SSX4 and BCNP1; SSX4 and SERPINB3; KLK2 and EpCAM; KLK2 and PBP; KLK2 and SPDEF; KLK2 and SSX2; KLK2 and EGFR; KLK2 and MMP7; KLK2 and BCNP1; KLK2 and SERPINB3; PBP and EGFR; PBP and EpCAM; PBP and SPDEF; PBP and SSX2; PBP and SERPINB3; PBP and MMP7; PBP and BCNP1; EpCAM and SPDEF; EpCAM and SSX2; EpCAM and SERPINB3; EpCAM and EGFR; EpCAM and MMP7; EpCAM and BCNP1; SPDEF and SSX2; SPDEF and SERPINB3; SPDEF and EGFR; SPDEF and MMP7; SPDEF and BCNP1; SSX2 and EGFR; SSX2 and MMP7; SSX2 and BCNP1; SSX2 and SERPINB3; SERPINB3 and EGFR; SERPINB3 and MMP7; SERPINB3 and BCNP1; EGFR and MMP7; EGFR and BCNP1; MMP7 and BCNP1; and a combination thereof. In a preferred embodiment, the detector agent comprises an EpCAM detector. In some embodiments, the detector agent recognizes one or more of a tetraspanin, CD9, CD63, CD81, CD63, CD9, CD81, CD82, CD37, CD53, Rab-5b, Annexin V, MFG-E8, or a protein in Table 3. In another embodiment, the detector agent recognizes one or more of CD9, CD63, CD81, PSMA, PCSA, B7H3, EpCam, ADAM-10, BCNP, EGFR, IL1B, KLK2, MMP7, p53, PBP, SERPINB3, SPDEF, SSX2, and SSX4. When using multiple capture agents, the assay can be multiplexed with a single detector agent. Alternately, each capture agent can be paired with a different detector agent. The biosignature can be used to characterize a prostate cancer.

In an embodiment, the one or more pair of capture and detector agents comprises binding agent pairs selected from the group consisting of EpCam-EpCam, EpCam-KLK2, EpCam-PBP, EpCam-SPDEF, EpCam-SSX2, EpCam-SSX4, EpCam-EGFR, and a combination thereof. The EpCAM may be the target of the detector agent. The biosignature can be used to characterize a prostate cancer.

In an embodiment, the one or more pair of capture and detector agents comprises binding agents to EpCam-EpCam.

In an embodiment, the one or more pair of capture and detector agents comprises binding agents to EpCam-KLK2.

In an embodiment, the one or more pair of capture and detector agents comprises binding agents to EpCam-PBP.

In an embodiment, the one or more pair of capture and detector agents comprises binding agents to EpCam-SPDEF.

In an embodiment, the one or more pair of capture and detector agents comprises binding agents to EpCam-SSX2.

In an embodiment, the one or more pair of capture and detector agents comprises binding agents to EpCam-SSX4.

In an embodiment, the one or more pair of capture and detector agents comprises binding agents to EpCam-EGFR.

In an aspect, the invention provides a method of identifying a biosignature by assessing biomarker complexes. In an aspect, the method comprises isolating one or more nucleic acid-protein complex from a biological sample; determining a presence or level of one or more nucleic acid biomarker with the one or more nucleic acid-protein complex; and identifying a biosignature comprising the presence or level of the one or more nucleic acid biomarker. In some embodiments, the biosignature may also comprise the presence or level of one or more protein or other component of the complex. The nucleic acid-protein complex may be isolated from the biological sample using methodology disclosed herein or known in the art. For example, the complex may be isolated by affinity selection such as by immunoprecipitation, column chromatography or flow cytometry, using a binding agent to a component of the complex. Binding agents can be as described herein, e.g., an antibody or aptamer to a protein component of the complex. In some embodiments, the method further comprises comparing the biosignature to a reference biosignature, wherein the comparison is used to characterize a cancer, including the cancers disclosed herein or known in the art. The reference biosignature can be from a subject without the cancer. The reference biosignature can also be from the subject, e.g., from normal adjacent tissue or from a sample taken at another point in time. Various ways of characterizing a cancer are disclosed herein. For example, characterizing the cancer may comprise identifying the presence or risk of the cancer in a subject, or identifying the cancer in a subject as metastatic or aggressive. The comparing step comprises determining whether the biosignature is altered relative to the reference biosignature, thereby providing a prognostic, diagnostic or theranostic characterization for the cancer. The biological sample comprises a bodily fluid, including without limitation the bodily fluids disclosed herein. For example, the bodily fluid may comprise urine, blood or a blood derivative.

In an embodiment, the nucleic acid-protein complex comprises one or more protein, e.g., 1, 2, 3, 4, 5, 6, 7, 8, 9 or 10 or more proteins, selected from the group consisting of one or more Argonaute family member, Ago1, Ago2, Ago3, Ago4, GW182 (TNRC6A), TNRC6B, TNRC6C, HNRNPA2B1, HNRPAB, ILF2, NCL (Nucleolin), NPM1 (Nucleophosmin), RPL10A, RPL5, RPLP1, RPS12, RPS19, SNRPG, TROVE2, apolipoprotein, apolipoprotein A, apo A-I, apo A-II, apo A-IV, apo A-V, apolipoprotein B, apo B48, apo B100, apolipoprotein C, apo C-I, apo C-II, apo apo C-IV, apolipoprotein D (ApoD), apolipoprotein E (ApoE), apolipoprotein H (ApoH), apolipoprotein L, APOL1, APOL2, APOL3, APOL4, APOL5, APOL6, APOLD1, and a combination thereof. For example, the nucleic acid-protein complex may comprise one or more protein selected from the group consisting of one or more Argonaute family member, Ago1, Ago2, Ago3, Ago4, GW182 (TNRC6A), and a combination thereof. The nucleic acid-protein complex comprises one or more protein selected from the group consisting of Ago2, Apolipoprotein I, GW182 (TNRC6A), and a combination thereof.

In embodiments, the one or more nucleic acid in the nucleic acid-protein complex comprises one or more microRNA. For example, the one or more microRNA, e.g., 1, 2, 3, 4, 5, 6, 7, 8, 9, 10, 20, 30, 40, 50 or more microRNA, can be a microRNA in Table 5. The one or more microRNA may comprise one or more microRNA, e.g., 1, 2, 3, 4, 5 or 6 microRNA, selected from the group consisting of miR-22, miR-16, miR-148a, miR-92a, miR-451, let7a, and a combination thereof. The one or more microRNA may be assessed in order to characterize, e.g., diagnose, prognose or theranose, a cancer including without limitation a prostate cancer.

In an embodiment, the nucleic acid-protein complex comprises one or more protein selected from the group consisting of Ago2, Apolipoprotein I, GW182 (TNRC6A), and a combination thereof; and the one or more microRNA comprises one or more microRNA selected from the group consisting of miR-16 and miR-92a, and a combination thereof. The one or more microRNA may be assessed in order to characterize a prostate cancer.

The invention further provides a method of determining a biosignature comprising detecting nucleic acids in microvesicle population of interest. The vesicle population can be a whole population in a biological sample, or a subpopulation such as a subpopulation having certain surface antigens. The method comprises detecting one or more protein biomarker in a microvesicle population from a biological sample; determining a presence or level of one or more one or more nucleic acid biomarker associated with the detected microvesicle population; and identifying a biosignature comprising the presence or level of the one or more nucleic acid. Techniques for detecting microvesicle populations, detecting proteins, and assessing nucleic acids can be disclosed herein or as known in the art. For example, the microvesicles can be isolated by affinity selection against the one or more protein, and nucleic acid can be isolated from the selected microvesicles. The level of the one or more one or more nucleic acid biomarker can be normalized to the level of the one or more protein biomarker or to the level of the microvesicle population. In some embodiments, the method further comprises comparing the biosignature to a reference biosignature, wherein the comparison is used to characterize a cancer, including the cancers disclosed herein or known in the art. The reference biosignature can be from a subject without the cancer. The reference biosignature can also be from the subject, e.g., from normal adjacent tissue or from a sample taken at another point in time. Various ways of characterizing a cancer are disclosed herein. For example, characterizing the cancer may comprise identifying the presence or risk of the cancer in a subject, or identifying the cancer in a subject as metastatic or aggressive. The comparing step comprises determining whether the biosignature is altered relative to the reference biosignature, thereby providing a prognostic, diagnostic or theranostic characterization for the cancer. The biological sample comprises a bodily fluid, including without limitation the bodily fluids disclosed herein. For example, the bodily fluid may comprise urine, blood or a blood derivative.

The proteins used for detecting one or more protein biomarker in a microvesicle population may comprise one or more biomarker disclosed herein, such as in Tables 3-5 or 9-11. For example, the one or more protein can be selected from the group consisting of PCSA, Ago2, CD9 and a combination thereof. For example, the one or more protein can be PCSA, Ago2, CD9, PCSA and Ago2, PCSA and CD9, Ago2 and CD9, or all of PCSA, Ago2 and CD9. Another general vesicle marker such as in Table 3, e.g., a tetraspanin such as CD63 or CD81 can be substituted for or used in addition to CD9. Such multiple biomarkers can be used to identify a microvesicle population having a certain origin. E.g., PCSA can identify prostate-derived vesicles while CD9 identifies vesicles apart from cellular debris. PCSA, PSMA, PSCA, KLK2 or PBP (prostate binding protein) can be used as a biomarker to characterize a prostate cancer.

The one or more nucleic acid biomarker may comprise one or more nucleic acid disclosed herein, such as in Table 5. In an embodiment, the one or more nucleic acid comprises one or more microRNA. For example, the one or more microRNA can be selected from 1, 2, 3, 4, 5 or 6 of miR-22, miR-16, miR-148a, miR-92a, miR-451, and let7a. In an embodiment, the one or more protein biomarker comprises PCSA and Ago2; and the one or more nucleic acid biomarker comprises miR-22. In another embodiment, the one or more protein biomarker comprises PCSA and/or CD9; and the one or more nucleic acid biomarker comprises miR-22. The method can be used to characterize a cancer such as a prostate cancer, e.g., to distinguish a cancer sample from a non-cancer sample.

In other embodiments, the one or more nucleic acid comprises mRNA. mRNA can be assessed as payload within microvesicles. For example, the one or more nucleic acid biomarker comprises a messenger RNA (mRNA) selected from Table 5. The mRNA may also be selected from any of Tables 22-24. In some embodiments, the one or more protein biomarker comprises PCSA; and the one or more nucleic acid biomarker comprises a messenger RNA (mRNA) selected from any of Tables 22-24. The method can be used to characterize a cancer such as a prostate cancer, e.g., to distinguish a cancer sample from a non-cancer sample.

The level of the one or more one or more nucleic acid biomarker can be normalized to the level of the one or more protein biomarker. In an embodiment, the biosignature comprises a score calculated from a ratio of the level of the one or more protein biomarker and one or more nucleic acid biomarker. For example, the level of the nucleic acids can be divided by the level of the proteins.

The score can be calculated from multiple proteins and multiple nucleic acids. In an embodiment, the one or more protein biomarker comprises PCSA and PSMA and the one or more nucleic acid biomarker comprises miR-22 and let7a. The method is used to characterize a prostate cancer, e.g., to distinguish a prostate cancer sample from a non-prostate cancer sample. The score may comprise taking the sum of: a) a first multiple of the level of miR-22 payload in the microvesicle subpopulation divided by the level of PCSA protein associated with the microvesicle subpopulation; b) a second multiple of the level of let7a payload in the microvesicle subpopulation divided by the level of PCSA protein associated with the microvesicle subpopulation; and c) a third multiple of the level of PSMA protein associated with the microvesicle subpopulation. The first, second and third multiples can be chosen to maximize the ability of the method to distinguish the prostate cancer. For example, the multiple can be about 0.0001, 0.001, 0.01, 0.1, 0.5, 1, 2, 3, 4, 5, 6, 7, 8, 9, 10, 100, 1000 or 10000. In an embodiment, the first multiple is 10, the second multiple is 10, and the third multiple is 1. The score can be an average of the sum as:

Score=Average(10*miR22/PCSA MFI,10*let-7a/PCSA MFI,PSMA MFI)

One of skill will appreciate that calculating the score may comprise a monotonic transformation of the sum. A similar scoring equation can be developed for other biomarkers in other settings, such as using alternate biomarkers to characterize other cancers.

By selecting a proper reference sample for comparison, the biosignatures identified can provide a diagnostic readout (e.g., reference sample is normal or non-disease), prognostic (e.g., reference sample is for poor or good disease outcome, aggressiveness or the like), or theranostic (e.g., reference sample is from a cohort responsive or non-responsive to selected treatment).

Additional biomarkers that can be used in the methods of the invention include those disclosed in International Patent Application PCT/US2012/025741, filed Feb. 17, 2012; International Patent Application PCT/US2011/048327, filed Aug. 18, 2011; International Patent Application PCT/US2011/026750, filed Mar. 1, 2011; and International Patent Application PCT/US2011/031479, filed Apr. 6, 2011; each of which is incorporated by reference herein in its entirety.

Gene Fusions

The one or more biomarkers assessed of vesicle, can be a gene fusion. A fusion gene is a hybrid gene created by the juxtaposition of two previously separate genes. This can occur by chromosomal translocation or inversion, deletion or via trans-splicing. The resulting fusion gene can cause abnormal temporal and spatial expression of genes, such as leading to abnormal expression of cell growth factors, angiogenesis factors, tumor promoters or other factors contributing to the neoplastic transformation of the cell and the creation of a tumor. Such fusion genes can be oncogenic due to the juxtaposition of: 1) a strong promoter region of one gene next to the coding region of a cell growth factor, tumor promoter or other gene promoting oncogenesis leading to elevated gene expression, or 2) due to the fusion of coding regions of two different genes, giving rise to a chimeric gene and thus a chimeric protein with abnormal activity.

An example of a fusion gene is BCR-ABL, a characteristic molecular aberration in ˜90% of chronic myelogenous leukemia (CML) and in a subset of acute leukemias (Kurzrock et al., Annals of Internal Medicine 2003; 138(10):819-830). The BCR-ABL results from a translocation between chromosomes 9 and 22. The translocation brings together the 5′ region of the BCR gene and the 3′ region of ABL1, generating a chimeric BCR-ABL1 gene, which encodes a protein with constitutively active tyrosine kinase activity (Mittleman et al., Nature Reviews Cancer 2007; 7(4):233-245). The aberrant tyrosine kinase activity leads to de-regulated cell signaling, cell growth and cell survival, apoptosis resistance and growth factor independence, all of which contribute to the pathophysiology of leukemia (Kurzrock et al., Annals of Internal Medicine 2003; 138(10):819-830).

Another fusion gene is IGH-MYC, a defining feature of ˜80% of Burkitt's lymphoma (Ferry et al. Oncologist 2006; 11(4):375-83). The causal event for this is a translocation between chromosomes 8 and 14, bringing the c-Myc oncogene adjacent to the strong promoter of the immunoglobin heavy chain gene, causing c-myc overexpression (Mittleman et al., Nature Reviews Cancer 2007; 7(4):233-245). The c-myc rearrangement is a pivotal event in lymphomagenesis as it results in a perpetually proliferative state. It has wide ranging effects on progression through the cell cycle, cellular differentiation, apoptosis, and cell adhesion (Ferry et al. Oncologist 2006; 11(4):375-83).

A number of recurrent fusion genes have been catalogued in the Mittleman database (cgap.nci.nih.gov/Chromosomes/Mitelman) and can be assessed in a vesicle, and used to characterize a phenotype. The gene fusion can be used to characterize a hematological malignancy or epithelial tumor. For example, TMPRSS2-ERG, TMPRSS2-ETV and SLC45A3-ELK4 fusions can be detected and used to characterize prostate cancer; and ETV6-NTRK3 and ODZ4-NRG1 for breast cancer.

Furthermore, assessing the presence or absence, or expression level of a fusion gene can be used to diagnosis a phenotype such as a cancer as well as a monitoring a therapeutic response to selecting a treatment. For example, the presence of the BCR-ABL fusion gene is a characteristic not only for the diagnosis of CML, but is also the target of the Novartis drug Imatinib mesylate (Gleevec), a receptor tyrosine kinase inhibitor, for the treatment of CML. Imatinib treatment has led to molecular responses (disappearance of BCR-ABL+ blood cells) and improved progression-free survival in BCR-ABL+CML patients (Kantarjian et al., Clinical Cancer Research 2007; 13(4):1089-1097).

Assessing a vesicle for the presence, absence, or expression level of a gene fusion can be of by assessing a heterogeneous population of vesicles for the presence, absence, or expression level of a gene fusion. Alternatively, the vesicle that is assessed can be derived from a specific cell type, such as cell-of-origin specific vesicle, as described above. Illustrative examples of use of fusions that can be assessed to characterize a phenotype include those described in International Patent Application Serial No. PCT/US2011/031479, entitled “Circulating Biomarkers for Disease” and filed Apr. 6, 2011, which application is incorporated by reference in its entirety herein.

Gene-Associated MiRNA Biomarkers

Illustrative examples of use of miRNA biomarkers known to interact with certain transcripts and that can be assessed to characterize a phenotype include those described in International Patent Application Serial No. PCT/US2011/031479, entitled “Circulating Biomarkers for Disease” and filed Apr. 6, 2011, which application is incorporated by reference in its entirety herein.

Nucleic Acid—Protein Complex Biomarkers

MicroRNAs in human plasma have been found associated with circulating microvesicles, Argonaute proteins, and HDL and LDL complexes. See, e.g., Arroyo et al., Argonaute2 complexes carry a population of circulating microRNAs independent of vesicles in human plasma. Proc Natl Acad Sci USA. 2011. 108:5003-08. Epub 2011 Mar. 7; Collino et al., Microvesicles derived from adult human bone marrow and tissue specific mesenchymal stem cells shuttle selected pattern of miRNAs. PLOS One. 2010 5(7):e11803. The Argonaute family of proteins plays a role in RNA interference (RNAi) gene silencing. Argonaute proteins bind short RNAs such as microRNAs (miRNAs) or short interfering RNAs (siRNAs), and repress the translation of their complementary mRNAs. They are also involved in transcriptional gene silencing (TGS), in which short RNAs known as antigene RNAs or agRNAs direct the transcriptional repression of complementary promoter regions. Argonaute family members include Argonaute 1 (“eukaryotic translation initiation factor 2C, 1”, EIF2C1, AGO1), Argonaute 2 (“eukaryotic translation initiation factor 2C, 2”, EIF2C2, AGO2), Argonaute 3 (“eukaryotic translation initiation factor 2C, 3”, EIF2C3, AGO3), and Argonaute 4 (“eukaryotic translation initiation factor 2C, 4”, EIF2C4, AGO4). Several Argonaute isotypes have been identified. Argonaute 2 is an effector protein within the RNA-Induced Silencing Complex (RISC) where it plays a role in the silencing of target messenger RNAs in the microRNA silencing pathway.

The protein GW182 associates with microvesicles and also has the capacity to bind all human Argonaute proteins (e.g., Ago1, Ago2, Ago3, Ago4) and their associated miRNAs. See, e.g., Gibbings et al., Multivesicular bodies associate with components of miRNA effector complexes and modulate miRNA activity, Nat Cell Biol 2009 11:1143-1149. Epub 2009 Aug. 16; Lazzaretti et al., The C-terminal domains of human TNRC6A, TNRC6B, and TNRC6C silence bound transcripts independently of Argonaute proteins. RNA. 2009 15:1059-66. Epub 2009 Apr. 21. GW182, which is encoded by the TNRC6A gene (trinucleotide repeat containing 6A), functions in post-transcriptional gene silencing through the RNA interference (RNAi) and microRNA pathways. TNRC6B and TNRC6C are also members of the trinucleotide repeat containing 6 family and play similar roles in gene silencing. GW182 associates with mRNAs and Argonaute proteins in cytoplasmic bodies known as GW-bodies or P-bodies. GW182 is involved in miRNA-dependent repression of translation and for siRNA-dependent endonucleolytic cleavage of complementary mRNAs by argonaute family proteins.

In an aspect, the invention provides a method of characterizing a phenotype comprising analyzing nucleic acid—protein complex biomarkers. As used herein, a nucleic acid—protein complex comprises at least one nucleic acid and at least one protein, and can also include other components such as lipids. A nucleic acid—protein complex can be associated with a vesicle. In an embodiment, RNA—protein complexes are isolated and the levels of the associated RNAs are assessed, wherein the levels are used for characterizing the phenotype, e.g., providing a diagnosis, prognosis, theranosis, or other phenotype as described herein. The RNA can be microRNA. MicroRNAs have been found associated with vesicles and proteins. In some cases, this association may serve to protect miRNAs from degradation via RNAses or other factors. Content of various populations of microRNA can be assessed in a sample, including without limitation vesicle associated miRs, Ago-associated miRs, cell-of-origin vesicle associated miRs, circulating Ago-bound miRs, circulating HDL-bound miRs, and the total miR content.

The protein biomarker used to isolate the complexes can be one or more Argonaute protein, or other protein that associates with Argonaute family members. These include without limitation the Argonaute proteins Ago1, Ago2, Ago3, Ago4, and various isoforms thereof. The protein biomarker can be GW182 (TNRC6A), TNRC6B and/or TNRC6C. The protein biomarker can be a protein associated with a P-body or a GW-body, such as SW182, an argonaute, decapping enzyme or RNA helicase. See, e.g., Kulkami et al. On track with P-bodies. Biochem Soc Trans 2010, 38:242-251. The protein biomarker can also be one or more of HNRNPA2B1 (Heterogeneous nuclear ribonucleoprotein a2/b1), HNRPAB (Heterogeneous nuclear ribonucleoprotein A/B), ILF2 (Interleukin enhancer binding factor 2, 45 kda), NCL (Nucleolin), NPM1 (Nucleophosmin (nucleolar phosphoprotein b23, numatrin)), RPL10A (Ribosomal protein 110a), RPL5 (Ribosomal protein 15), RPLP1 (Ribosomal protein, large, p1), RPS12 (Ribosomal protein s12), RPS19 (Ribosomal protein s19), SNRPG (Small nuclear ribonucleoprotein polypeptide g), TROVE2 (Trove domain family, member 2). See Wang et al., Export of microRNAs and microRNA-protective protein by mammalian cells. Nucleic Acids Res. 38:7248-59. Epub 2010 Jul. 7. The protein biomarker can also be an apolipoprotein, which are proteins that bind to lipids (oil-soluble substances such as fat and cholesterol) to form lipoproteins, which transport the lipids through the lymphatic and circulatory systems. See Vickers et al., MicroRNAs are transported in plasma and delivered to recipient cells by high-density lipoproteins, Nat Cell Biol 2011 13:423-33, Epub 2011 Mar. 20. The apolipoprotein can be apolipoprotein A (including apo A-I, apo A-II, apo A-IV, and apo A-V), apolipoprotein B (including apo B48 and apo B100), apolipoprotein C (including apo C-I, apo C-II, apo C-III, and apo C-IV), apolipoprotein D (ApoD), apolipoprotein E (ApoE), apolipoprotein H (ApoH), or a combination thereof. The apolipoprotein can be apolipoprotein L, including APOL1, APOL2, APOL3, APOL4, APOL5, APOL6, APOLD1, or a combination thereof. Apolipoprotein L (Apo L) belongs to the high density lipoprotein family that plays a central role in cholesterol transport. The protein biomarker can be a component of a lipoprotein, such as a component of a chylomicron, very low density lipoprotein (VLDL), intermediate density lipoprotein (IDL), low density lipoprotein (LDL) and/or high density lipoprotein (HDL). In an embodiment, the protein biomarker is a component of a LDL or HDL. The component can be ApoE. The component can be ApoA1. The protein biomarker can be a general vesicle marker, such as a tetraspanin or other protein listed in Table 3, including without limitation CD9, CD63 and/or CD81. The protein biomarker can be a cancer marker such as EpCam, B7H3 and/or CD24. The protein biomarker can be a tissue specific biomarker, such as the prostate biomarkers PSCA, PCSA and/or PSMA. Combinations of these or other useful protein biomarkers can be used to isolate specific populations of complexes of interest.

The nucleic acid—protein complexes can be isolated by using a binding agent to one or more component of the complexes. Various techniques for isolating proteins are known to those of skill in the art and/or presented herein, including without limitation affinity isolation, immunocapture, immunoprecipitation, and flow cytometry. The binding agent can be any appropriate binding agent, including those described herein such as the one or more binding agent comprises a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof. In an embodiment, the binding agent comprises an antibody, antibody conjugate, antibody fragment, and/or aptamer. For additional methods of assessing protein—nucleic acid complexes that can be used with the subject invention, see also Wang et al., Export of microRNAs and microRNA-protective protein by mammalian cells. Nucleic Acids Res. 38:7248-59. Epub 2010 Jul. 7; Keene et al., RIP-Chip: the isolation and identification of mRNAs, microRNAs and protein components of ribonucleoprotein complexes from cell extracts. Nat Protoc 2006 1:302-07; Hafner, Transcriptome-wide identification of RNA-binding protein and microRNA target sites by PAR-CLIP. Cell 2010 141:129-41.

The present invention further provides a method of identifying miRNAs that are found in complex with proteins. In one embodiment, a population of protein—nucleic acid complexes is isolated as described above. The miRNA content of the population is assessed. This method can be used on various samples of interest (e.g., diseased, non-diseased, responder, non-responder) and the miRNA content in the samples can be compared to identify miRNAs that differentiate between the samples. Methods of detecting miRNA are provided herein (arrays, per, etc). The identified miRNAs can be used to characterize a phenotype according to the methods herein. For example, the samples used for discovery can be cancer and non-cancer plasma samples. Protein-complexed miRNAs can be identified that distinguish between the cancer and non-cancer samples, and the distinguishing miRNAs can be assessed in order to detect a cancer in a plasma sample.

The present invention also provides a method of distinguishing microRNA payload within vesicles by removing non-payload miRs from a vesicle-containing sample, then assessing the miR content within the vesicles. miRs can be removed from the sample using RNAses or other entities that degrade miRNA. In some embodiments, the sample is treated with an agent to remove microRNAs from protein complexes prior to the RNAse treatment. The agent can be an enzyme that degrades protein, e.g., a proteinase such as Proteinase K or Trypsin, or any other appropriate enzyme. The method can be used to characterize a phenotype according to the methods herein by assessing the microRNA fraction contained with vesicles apart from free miRNA or miRNA in circulating protein complexes.

### Biomarker Detection

The compositions and methods of the invention can be used to assess any useful biomarkers in a biological sample for charactering a phenotype associated with the sample. Such biomarkers include all sorts of biological entities such as proteins, nucleic acids, lipids, carbohydrates, complexes of any thereof, and microvesicles. Various molecules associated with a microvesicle surface or enclosed within the microvesicle (referred to herein as “payload”) can serve as biomarkers. The microvesicles themselves can also be used as biomarkers.

A biosignature can be detected qualitatively or quantitatively by detecting a presence, level or concentration of a circulating biomarker, e.g., a microRNA, protein, vesicle or other biomarker, as disclosed herein. These biosignature components can be detected using a number of techniques known to those of skill in the art. For example, a biomarker can be detected by microarray analysis, polymerase chain reaction (PCR) (including PCR-based methods such as real time polymerase chain reaction (RT-PCR), quantitative real time polymerase chain reaction (Q-PCR/qPCR) and the like), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or combinations thereof. A biomarker, such as a nucleic acid, can be amplified prior to detection. A biomarker can also be detected by immunoassay, immunoblot, immunoprecipitation, enzyme-linked immunosorbent assay (ELISA; EIA), radioimmunoassay (RIA), flow cytometry, or electron microscopy (EM).

Biosignatures can be detected using capture agents and detection agents, as described herein. A capture agent can comprise an antibody, aptamer or other entity which recognizes a biomarker and can be used for capturing the biomarker. Biomarkers that can be captured include circulating biomarkers, e.g., a protein, nucleic acid, lipid or biological complex in solution in a bodily fluid. Similarly, the capture agent can be used for capturing a vesicle. A detection agent can comprise an antibody or other entity which recognizes a biomarker and can be used for detecting the biomarker vesicle, or which recognizes a vesicle and is useful for detecting a vesicle. In some embodiments, the detection agent is labeled and the label is detected, thereby detecting the biomarker or vesicle. The detection agent can be a binding agent, e.g., an antibody or aptamer. In other embodiments, the detection agent comprises a small molecule such as a membrane protein labeling agent. See, e.g., the membrane protein labeling agents disclosed in Alroy et al., US. Patent Publication US 2005/0158708. In an embodiment, vesicles are isolated or captured as described herein, and one or more membrane protein labeling agent is used to detect the vesicles. In many cases, the antigen or other vesicle-moiety that is recognized by the capture and detection agents are interchangeable. As a non-limiting example, consider a vesicle having a cell-of-origin specific antigen on its surface and a cancer-specific antigen on its surface. In one instance, the vesicle can be captured using an antibody to the cell-of-origin specific antigen, e.g., by tethering the capture antibody to a substrate, and then the vesicle is detected using an antibody to the cancer-specific antigen, e.g., by labeling the detection antibody with a fluorescent dye and detecting the fluorescent radiation emitted by the dye. In another instance, the vesicle can be captured using an antibody to the cancer specific antigen, e.g., by tethering the capture antibody to a substrate, and then the vesicle is detected using an antibody to the cell-of-origin specific antigen, e.g., by labeling the detection antibody with a fluorescent dye and detecting the fluorescent radiation emitted by the dye.

In some embodiments, a same biomarker is recognized by both a capture agent and a detection agent. This scheme can be used depending on the setting. In one embodiment, the biomarker is sufficient to detect a vesicle of interest, e.g., to capture cell-of-origin specific vesicles. In other embodiments, the biomarker is multifunctional, e.g., having both cell-of-origin specific and cancer specific properties. The biomarker can be used in concert with other biomarkers for capture and detection as well.

One method of detecting a biomarker comprises purifying or isolating a heterogeneous population of vesicles from a biological sample, as described above, and performing a sandwich assay. A vesicle in the population can be captured with a capture agent. The capture agent can be a capture antibody, such as a primary antibody. The capture antibody can be bound to a substrate, for example an array, well, or particle. The captured or bound vesicle can be detected with a detection agent, such as a detection antibody. For example, the detection antibody can be for an antigen of the vesicle. The detection antibody can be directly labeled and detected. Alternatively, the detection agent can be indirectly labeled and detected, such as through an enzyme linked secondary antibody that can react with the detection agent. A detection reagent or detection substrate can be added and the reaction detected, such as described in PCT Publication No. WO2009092386. In an illustrative example wherein the capture agent binds Rab-5b and the detection agent binds or detects CD63 or caveolin-1, the capture agent can be an anti-Rab 5b antibody and the detection agent can be an anti-CD63 or anti-caveolin-1 antibody. In some embodiments, the capture agent binds CD9, PSCA, TNFR, CD63, B7H3, MFG-E8, EpCam, Rab, CD81, STEAP, PCSA, PSMA, or 5T4. For example, the capture agent can be an antibody to CD9, PSCA, TNFR, CD63, B7H3, MFG-E8, EpCam, Rab, CD81, STEAP, PCSA, PSMA, or 5T4. The capture agent can also be an antibody to MFG-E8, Annexin V, Tissue Factor, DR3, STEAP, epha2, TMEM211, unc93A, A33, CD24, NGAL, EpCam, MUC17, TROP2, or TETS. The detection agent can be an agent that binds or detects CD63, CD9, CD81, B7H3, or EpCam, such as a detection antibody or aptamer to CD63, CD9, CD81, B7H3, or EpCam. Various combinations of capture and/or detection agents can be used in concert. In an embodiment, the capture agents comprise PCSA, PSMA, B7H3 and optionally EpCam, and the detection agents comprise one or more general vesicle biomarker, e.g., a tetraspanin such as CD9, CD63 and CD81. In another embodiment, the capture agents comprise TMEM211 and CD24, and the detection agents comprise one or more tetraspanin such as CD9, CD63 and CD81. In another embodiment, the capture agents comprise CD66 and EpCam, and the detection agents comprise one or more tetraspanin such as CD9, CD63 and CD81. The capture agent and/or detection agent can be to an antigen comprising one or more of CD9, Erb2, Erb4, CD81, Erb3, MUC16, CD63, DLL4, HLA-Drpe, B7H3, IFNAR, 5T4, PCSA, MICB, PSMA, MFG-E8, Muc1, PSA, Muc2, Unc93a, VEGFR2, EpCAM, VEGF A, TMPRSS2, RAGE*, PSCA, CD40, Muc17, IL-17-RA, and CD80. For example, capture agent and/or detection agent can be to one or more of CD9, CD63, CD81, B7H3, PCSA, MFG-E8, MUC2, EpCam, RAGE and Muc17. Increasing numbers of such tetraspanins and/or other general vesicle markers can improve the detection signal in some cases. Proteins or other circulating biomarkers can also be detected using sandwich approaches. The captured vesicles can be collected and used to analyze the payload contained therein, e.g., mRNA, microRNAs, DNA and soluble protein.

In some embodiments, the capture agent binds or targets EpCam, B7H3, RAGE or CD24, and the one or more biomarkers detected on the vesicle are CD9 and/or CD63. In one embodiment, the capture agent binds or targets EpCam, and the one or more biomarkers detected on the vesicle are CD9, EpCam and/or CD81. The single capture agent can be selected from CD9, PSCA, TNFR, CD63, B7H3, MFG-E8, EpCam, Rab, CD81, STEAP, PCSA, PSMA, or 5T4. The single capture agent can also be an antibody to DR3, STEAP, epha2, TMEM211, unc93A, A33, CD24, NGAL, EpCam, MUC17, TROP2, MFG-E8, TF, Annexin V or TETS. In some embodiments, the single capture agent is selected from PCSA, PSMA, B7H3, CD81, CD9 and CD63.

In other embodiments, the capture agent targets PCSA, and the one or more biomarkers detected on the captured vesicle are B7H3 and/or PSMA. In other embodiments, the capture agent targets PSMA, and the one or more biomarkers detected on the captured vesicle are B7H3 and/or PCSA. In other embodiments, the capture agent targets B7H3, and the one or more biomarkers detected on the captured vesicle are PSMA and/or PCSA. In yet other embodiments, the capture agent targets CD63 and the one or more biomarkers detected on the vesicle are CD81, CD83, CD9 and/or CD63. The different capture agent and biomarker combinations disclosed herein can be used to characterize a phenotype, such as detecting, diagnosing or prognosing a disease, e.g., a cancer. In some embodiments, vesicles are analyzed to characterize prostate cancer using a capture agent targeting EpCam and detection of CD9 and CD63; a capture agent targeting PCSA and detection of B7H3 and PSMA; or a capture agent of CD63 and detection of CD81. In other embodiments, vesicles are used to characterize colon cancer using capture agent targeting CD63 and detection of CD63, or a capture agent targeting CD9 coupled with detection of CD63. One of skill will appreciate that targets of capture agents and detection agents can be used interchangeably. In an illustrative example, consider a capture agent targeting PCSA and detection agents targeting B7H3 and PSMA. Because all of these markers are useful for detecting PCa derived vesicles, B7H3 or PSMA could be targeted by the capture agent and PCSA could be recognized by a detection agent. For example, in some embodiments, the detection agent targets PCSA, and one or more biomarkers used to capture the vesicle comprise B7H3 and/or PSMA. In other embodiments, the detection agent targets PSMA, and the one or more biomarkers used to capture the vesicle comprise B7H3 and/or PCSA. In other embodiments, the detection agent targets B7H3, and the one or more biomarkers used to capture the vesicle comprise PSMA and/or PCSA. In some embodiments, the invention provides a method of detecting prostate cancer cells in bodily fluid using capture agents and/or detection agents to PSMA, B7H3 and/or PCSA. The bodily fluid can comprise blood, including serum or plasma. The bodily fluid can comprise ejaculate or sperm. In further embodiments, the methods of detecting prostate cancer further use capture agents and/or detection agents to CD81, CD83, CD9 and/or CD63. The method further provides a method of characterizing a GI disorder, comprising capturing vesicles with one or more of DR3, STEAP, epha2, TMEM211, unc93A, A33, CD24, NGAL, EpCam, MUC17, TROP2, and TETS, and detecting the captured vesicles with one or more general vesicle antigen, such as CD81, CD63 and/or CD9. Additional agents can improve the test performance, e.g., improving test accuracy or AUC, either by providing additional biological discriminatory power and/or by reducing experimental noise.

Techniques of detecting biomarkers for use with the invention include the use of a planar substrate such as an array (e.g., biochip or microarray), with molecules immobilized to the substrate as capture agents that facilitate the detection of a particular biosignature. The array can be provided as part of a kit for assaying one or more biomarkers or vesicles. A molecule that identifies the biomarkers described above and shown in FIG. 1 or 3-60 of International Patent Application Serial No. PCT/US2011/031479, entitled “Circulating Biomarkers for Disease” and filed Apr. 6, 2011, which application is incorporated by reference in its entirety herein, can be included in an array for detection and diagnosis of diseases including presymptomatic diseases. In some embodiments, an array comprises a custom array comprising biomolecules selected to specifically identify biomarkers of interest. Customized arrays can be modified to detect biomarkers that increase statistical performance, e.g., additional biomolecules that identifies a biosignature which lead to improved cross-validated error rates in multivariate prediction models (e.g., logistic regression, discriminant analysis, or regression tree models). In some embodiments, customized array(s) are constructed to study the biology of a disease, condition or syndrome and profile biosignatures in defined physiological states. Markers for inclusion on the customized array be chosen based upon statistical criteria, e.g., having a desired level of statistical significance in differentiating between phenotypes or physiological states. In some embodiments, standard significance of p-value=0.05 is chosen to exclude or include biomolecules on the microarray. The p-values can be corrected for multiple comparisons. As an illustrative example, nucleic acids extracted from samples from a subject with or without a disease can be hybridized to a high density microarray that binds to thousands of gene sequences. Nucleic acids whose levels are significantly different between the samples with or without the disease can be selected as biomarkers to distinguish samples as having the disease or not. A customized array can be constructed to detect the selected biomarkers. In some embodiments, customized arrays comprise low density microarrays, which refer to arrays with lower number of addressable binding agents, e.g., tens or hundreds instead of thousands. Low density arrays can be formed on a substrate. In some embodiments, customizable low density arrays use PCR amplification in plate wells, e.g., TaqMan® Gene Expression Assays (Applied Biosystems by Life Technologies Corporation, Carlsbad, Calif.).

A planar array generally contains addressable locations (e.g., pads, addresses, or micro-locations) of biomolecules in an array format. The size of the array will depend on the composition and end use of the array. Arrays can be made containing from 2 different molecules to many thousands. Generally, the array comprises from two to as many as 100,000 or more molecules, depending on the end use of the array and the method of manufacture. A microarray for use with the invention comprises at least one biomolecule that identifies or captures a biomarker present in a biosignature of interest, e.g., a microRNA or other biomolecule or vesicle that makes up the biosignature. In some arrays, multiple substrates are used, either of different or identical compositions. Accordingly, planar arrays may comprise a plurality of smaller substrates.

The present invention can make use of many types of arrays for detecting a biomarker, e.g., a biomarker associated with a biosignature of interest. Useful arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). These arrays are described in more detail above. In some embodiments, microarrays comprise biochips that provide high-density immobilized arrays of recognition molecules (e.g., antibodies), where biomarker binding is monitored indirectly (e.g., via fluorescence). FIG. 2A shows an illustrative configuration in which capture agents, e.g., antibodies or aptamers, against a vesicle antigen of interest are tethered to a surface. The captured vesicles are then detected using detector agents, e.g., antibodies or aptamers, against the same or different vesicle antigens of interest. Fluorescent detectors are shown. Other detectors can be used similarly, e.g., enzymatic reaction, detectable nanoparticles, radiolabels, and the like. In other embodiments, an array comprises a format that involves the capture of proteins by biochemical or intermolecular interaction, coupled with detection by mass spectrometry (MS). The vesicles can be eluted from the surface and the payload therein, e.g., microRNA, can be analyzed.

An array or microarray that can be used to detect one or more biomarkers of a biosignature can be made according to the methods described in U.S. Pat. Nos. 6,329,209; 6,365,418; 6,406,921; 6,475,808; and 6,475,809, and U.S. patent application Ser. No. 10/884,269, each of which is herein incorporated by reference in its entirety. Custom arrays to detect specific selections of sets of biomarkers described herein can be made using the methods described in these patents. Commercially available microarrays can also be used to cany out the methods of the invention, including without limitation those from Affymetrix (Santa Clara, Calif.), Illumina (San Diego, Calif.), Agilent (Santa Clara, Calif.), Exiqon (Denmark), or Invitrogen (Carlsbad, Calif.). Custom and/or commercial arrays include arrays for detection proteins, nucleic acids, and other biological molecules and entities (e.g., cells, vesicles, virii) as described herein.

In some embodiments, molecules to be immobilized on an array comprise proteins or peptides. One or more types of proteins may be immobilized on a surface. In certain embodiments, the proteins are immobilized using methods and materials that minimize the denaturing of the proteins, that minimize alterations in the activity of the proteins, or that minimize interactions between the protein and the surface on which they are immobilized.

Array surfaces useful may be of any desired shape, form, or size. Non-limiting examples of surfaces include chips, continuous surfaces, curved surfaces, flexible surfaces, films, plates, sheets, or tubes. Surfaces can have areas ranging from approximately a square micron to approximately 500 cm². The area, length, and width of surfaces may be varied according to the requirements of the assay to be performed. Considerations may include, for example, ease of handling, limitations of the material(s) of which the surface is formed, requirements of detection systems, requirements of deposition systems (e.g., arrayers), or the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within microwell plates having any number of wells. In such embodiments, the bottoms of the wells may serve as surfaces for the formation of arrays, or arrays may be formed on other surfaces and then placed into wells. In certain embodiments, such as where a surface without wells is used, binding islands may be formed or molecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer necessary.

In some embodiments, the immobilized molecules can bind to one or more biomarkers or vesicles present in a biological sample contacting the immobilized molecules. In some embodiments, the immobilized molecules modify or are modified by molecules present in the one or more vesicles contacting the immobilized molecules. Contacting the sample typically comprises overlaying the sample upon the array.

Modifications or binding of molecules in solution or immobilized on an array can be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces are measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM), scanning force microscopy (SFM) or scanning electron microscopy (SEM); and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8):363-369 (1999), and references cited therein; Lakowicz J R, Principles of Fluorescence Spectroscopy, 2nd Edition, Plenum Press (1999), or Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, each of which is herein incorporated by reference in its entirety.

Microarray technology can be combined with mass spectroscopy (MS) analysis and other tools. Electrospray interface to a mass spectrometer can be integrated with a capillary in a microfluidics device. For example, one commercially available system contains eTag reporters that are fluorescent labels with unique and well-defined electrophoretic mobilities; each label is coupled to biological or chemical probes via cleavable linkages. The distinct mobility address of each eTag reporter allows mixtures of these tags to be rapidly deconvoluted and quantitated by capillary electrophoresis. This system allows concurrent gene expression, protein expression, and protein function analyses from the same sample Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety.

A biochip can include components for a microfluidic or nanofluidic assay. A microfluidic device can be used for isolating or analyzing biomarkers, such as determining a biosignature. Microfluidic systems allow for the miniaturization and compartmentalization of one or more processes for isolating, capturing or detecting a vesicle, detecting a microRNA, detecting a circulating biomarker, detecting a biosignature, and other processes. The microfluidic devices can use one or more detection reagents in at least one aspect of the system, and such a detection reagent can be used to detect one or more biomarkers. In one embodiment, the device detects a biomarker on an isolated or bound vesicle. Various probes, antibodies, proteins, or other binding agents can be used to detect a biomarker within the microfluidic system. The detection agents may be immobilized in different compartments of the microfluidic device or be entered into a hybridization or detection reaction through various channels of the device.

A vesicle in a microfluidic device can be lysed and its contents detected within the microfluidic device, such as proteins or nucleic acids, e.g., DNA or RNA such as miRNA or mRNA. The nucleic acid may be amplified prior to detection, or directly detected, within the microfluidic device. Thus microfluidic system can also be used for multiplexing detection of various biomarkers. In an embodiment, vesicles are captured within the microfluidic device, the captured vesicles are lysed, and a biosignature of microRNA from the vesicle payload is determined. The biosignature can further comprise the capture agent used to capture the vesicle.

Novel nanofabrication techniques are opening up the possibilities for biosensing applications that rely on fabrication of high-density, precision arrays, e.g., nucleotide-based chips and protein arrays otherwise know as heterogeneous nanoarrays. Nanofluidics allows a further reduction in the quantity of fluid analyte in a microchip to nanoliter levels, and the chips used here are referred to as nanochips. (See, e.g., Unger M et al., Biotechniques 1999; 27(5):1008-14, Kartalov E P et al., Biotechniques 2006; 40(1):85-90, each of which are herein incorporated by reference in their entireties.) Commercially available nanochips currently provide simple one step assays such as total cholesterol, total protein or glucose assays that can be run by combining sample and reagents, mixing and monitoring of the reaction. Gel-free analytical approaches based on liquid chromatography (LC) and nanoLC separations (Cutillas et al. Proteomics, 2005; 5:101-112 and Cutillas et al., Mol Cell Proteomics 2005; 4:1038-1051, each of which is herein incorporated by reference in its entirety) can be used in combination with the nanochips.

An array suitable for identifying a disease, condition, syndrome or physiological status can be included in a kit. A kit can include, as non-limiting examples, one or more reagents useful for preparing molecules for immobilization onto binding islands or areas of an array, reagents useful for detecting binding of a vesicle to immobilized molecules, and instructions for use.

Further provided herein is a rapid detection device that facilitates the detection of a particular biosignature in a biological sample. The device can integrate biological sample preparation with polymerase chain reaction (PCR) on a chip. The device can facilitate the detection of a particular biosignature of a vesicle in a biological sample, and an example is provided as described in Pipper et al., Angewandte Chemie, 47(21), p. 3900-3904 (2008), which is herein incorporated by reference in its entirety. A biosignature can be incorporated using micro-/nano-electrochemical system (MEMS/NEMS) sensors and oral fluid for diagnostic applications as described in Li et al., Adv Dent Res 18(1): 3-5 (2005), which is herein incorporated by reference in its entirety.

As an alternative to planar arrays, assays using particles, such as bead based assays as described herein, can be used in combination with flow cytometry. Multiparametric assays or other high throughput detection assays using bead coatings with cognate ligands and reporter molecules with specific activities consistent with high sensitivity automation can be used. In a bead based assay system, a binding agent for a biomarker or vesicle, such as a capture agent (e.g. capture antibody), can be immobilized on an addressable microsphere. Each binding agent for each individual binding assay can be coupled to a distinct type of microsphere (i.e., microbead) and the assay reaction takes place on the surface of the microsphere, such as depicted in FIG. 2B. A binding agent for a vesicle can be a capture antibody or aptamer coupled to a bead. Dyed microspheres with discrete fluorescence intensities are loaded separately with their appropriate binding agent or capture probes. The different bead sets carrying different binding agents can be pooled as necessary to generate custom bead arrays. Bead arrays are then incubated with the sample in a single reaction vessel to perform the assay. Examples of microfluidic devices that may be used, or adapted for use with the invention, include but are not limited to those described herein.

Product formation of the biomarker with an immobilized capture molecule or binding agent can be detected with a fluorescence based reporter system (see for example, FIG. 2A-B). The biomarker can either be labeled directly by a fluorophore or detected by a second fluorescently labeled capture biomolecule. The signal intensities derived from captured biomarkers can be measured in a flow cytometer. The flow cytometer can first identify each microsphere by its individual color code. For example, distinct beads can be dyed with discrete fluorescence intensities such that each bead with a different intensity has a different binding agent. The beads can be labeled or dyed with at least 2 different labels or dyes. In some embodiments, the beads are labeled with at least 3, 4, 5, 6, 7, 8, 9, or 10 different labels. The beads with more than one label or dye can also have various ratios and combinations of the labels or dyes. The beads can be labeled or dyed externally or may have intrinsic fluorescence or signaling labels.

The amount of captured biomarkers on each individual bead can be measured by the second color fluorescence specific for the bound target. This allows multiplexed quantitation of multiple targets from a single sample within the same experiment. Sensitivity, reliability and accuracy are compared or can be improved to standard microtiter ELISA procedures. An advantage of a bead-based system is the individual coupling of the capture biomolecule or binding agent for a vesicle to distinct microspheres provides multiplexing capabilities. For example, as depicted in FIG. 2C, a combination of 5 different biomarkers to be detected (detected by binding agents such as antibodies or aptamers to antigens to CD63, CD9, CD81, B7H3, and EpCam) and 20 biomarkers for which to capture a vesicle, (using capture agents such as antibodies or aptamers to antigens to CD9, PSCA, TNFR, CD63, B7H3, MFG-E8, EpCam, Rab, CD81, STEAP, PCSA, PSMA, 5T4, and/or CD24) can result in approximately 100 combinations to be detected. As shown in FIG. 2C as “EpCam 2x,” “CD63 2X,” multiple binding agents to a single target can be used to probe detection against various epitopes. In another example, multiplex analysis comprises capturing a vesicle using a binding agent to CD24 and detecting the captured vesicle using a binding agent for CD9, CD63, and/or CD81. The captured vesicles can be detected using a detection agent such as an antibody or aptamer. The detection agents can be labeled directly or indirectly, as described herein.

The methods of the invention can comprise multiplex analysis of at least 2, 3, 4, 5, 6, 7, 8, 9, 10, 11, 12, 13, 14, 15, 16, 17, 18, 19, 20, 25, 50, 75 or 100 different biomarkers. For example, an assay of a heterogeneous population of vesicles can be performed with a plurality of particles that are differentially labeled. There can be at least 2, 3, 4, 5, 6, 7, 8, 9, 10, 11, 12, 13, 14, 15, 16, 17, 18, 19, 20, 25, 50, 75 or 100 differentially labeled particles. The particles may be externally labeled, such as with a tag, or they may be intrinsically labeled. Each differentially labeled particle can be coupled to a capture agent, such as a binding agent, for a vesicle, resulting in capture of a vesicle. The multiple capture agents can be selected to characterize a phenotype of interest, including capture agents against general vesicle biomarkers, cell-of-origin specific biomarkers, and disease biomarkers. One or more biomarkers of the captured vesicle can then be detected by a plurality of binding agents. The binding agent can be directly labeled to facilitate detection. Alternatively, the binding agent is labeled by a secondary agent. For example, the binding agent may be an antibody for a biomarker on the vesicle. The binding agent is linked to biotin. A secondary agent comprises streptavidin linked to a reporter and can be added to detect the biomarker. In some embodiments, the captured vesicle is assayed for at least 2, 3, 4, 5, 6, 7, 8, 9, 10, 11, 12, 13, 14, 15, 16, 17, 18, 19, 20, 25, 50, 75 or 100 different biomarkers. For example, multiple detectors, i.e., detection of multiple biomarkers of a captured vesicle or population of vesicles, can increase the signal obtained, permitted increased sensitivity, specificity, or both, and the use of smaller amounts of samples. Detection can be with more than one biomarker, including without limitation more than one general vesicle marker such as in Table 3. Use of multiple detectors may be used to amplify the signal as desired.

An immunoassay based method (e.g., sandwich assay) can be used to detect a biomarker of a vesicle. An example includes ELISA. A binding agent can be bound to a well. For example, a binding agent such as an aptamer or antibody to an antigen of a vesicle can be attached to a well. A biomarker on the captured vesicle can be detected based on the methods described herein. FIG. 2A shows an illustrative schematic for a sandwich-type of immunoassay. The capture agent can be against a vesicle antigen of interest, e.g., a general vesicle biomarker, a cell-of-origin marker, or a disease marker. In the figure, the captured vesicles are detected using fluorescently labeled binding agent (detection agent) against vesicle antigens of interest. Multiple capture binding agents can be used, e.g., in distinguishable addresses on an array or different wells of an immunoassay plate. The detection binding agents can be against the same antigen as the capture binding agent, or can be directed against other markers. The capture binding agent can be any useful binding agent, e.g., tethered aptamers, antibodies or lectins, and/or the detector antibodies can be similarly substituted, e.g., with detectable (e.g., labeled) aptamers, antibodies, lectins or other binding proteins or entities. In an embodiment, one or more capture agents to a general vesicle biomarker, a cell-of-origin marker, and/or a disease marker are used along with detection agents against general vesicle biomarker, such as tetraspanin molecules including without limitation one or more of CD9, CD63 and CD81, or other markers in Table 3 herein. Examples of microvesicle surface antigens are disclosed herein, e.g. in Tables 3, 4 or 5, or are known in the art, and examples useful in methods and compositions of the invention are disclosed of International Patent Application Serial No. PCT/US2011/031479, entitled “Circulating Biomarkers for Disease” and filed Apr. 6, 2011.

FIG. 2D presents an illustrative schematic for analyzing vesicles according to the methods of the invention. Capture agents are used to capture vesicles, detectors are used to detect the captured vesicles, and the level or presence of the captured and detected microvesicles is used to characterize a phenotype. Capture agents, detectors and characterizing phenotypes can be any of those described herein. For example, capture agents include antibodies or aptamers tethered to a substrate that recognize a vesicle antigen of interest, detectors include labeled antibodies or aptamers to a vesicle antigen of interest, and characterizing a phenotype includes a diagnosis, prognosis, or theranosis of a disease. In the scheme shown in FIG. 2D i), a population of vesicles is captured with one or more capture agents against general vesicle biomarkers (200). The captured vesicles are then labeled with detectors against cell-of-origin biomarkers (201) and/or disease specific biomarkers (202). If only cell-of-origin detectors are used (201), the biosignature used to characterize the phenotype (203) can include the general vesicle markers (200) and the cell-of-origin biomarkers (201). If only disease detectors are used (202), the biosignature used to characterize the phenotype (203) can include the general vesicle markers (200) and the disease biomarkers (202). Alternately, detectors are used to detect both cell-of-origin biomarkers (201) and disease specific biomarkers (202). In this case, the biosignature used to characterize the phenotype (203) can include the general vesicle markers (200), the cell-of-origin biomarkers (201) and the disease biomarkers (202). The biomarkers combinations are selected to characterize the phenotype of interest and can be selected from the biomarkers and phenotypes described herein, e.g., in Tables 3, 4 or 5.

In the scheme shown in FIG. 2D ii), a population of vesicles is captured with one or more capture agents against cell-of-origin biomarkers (210) and/or disease biomarkers (211). The captured vesicles are then detected using detectors against general vesicle biomarkers (212). If only cell-of-origin capture agents are used (210), the biosignature used to characterize the phenotype (213) can include the cell-of-origin biomarkers (210) and the general vesicle markers (212). If only disease biomarker capture agents are used (211), the biosignature used to characterize the phenotype (213) can include the disease biomarkers (211) and the general vesicle biomarkers (212). Alternately, capture agents to one or more cell-of-origin biomarkers (210) and one or more disease specific biomarkers (211) are used to capture vesicles. In this case, the biosignature used to characterize the phenotype (213) can include the cell-of-origin biomarkers (210), the disease biomarkers (211), and the general vesicle markers (213). The biomarkers combinations are selected to characterize the phenotype of interest and can be selected from the biomarkers and phenotypes described herein.

The methods of the invention comprise capture and detection of microvesicles of interest using any combination of useful biomarkers. For example, a microvesicle population can be captured using one or more binding agent to any desired combination of cell of origin, disease specific, or general vesicle markers. The captured microvesicles can then be detected using one or more binding agent to any desired combination of cell of origin, disease specific, or general vesicle markers. FIG. 2E represents a flow diagram of such configurations. Any one or more of a cell-of-origin biomarker (240), disease biomarkers (241), and general vesicle biomarker (242) is used to capture a microvesicle population. Thereafter, any one or more of a cell-of-origin biomarker (243), disease biomarkers (244), and general vesicle biomarker (245) is used to detect the captured microvesicle population. The biosignature of captured and detected microvesicles is then used to characterize a phenotype (246). The biomarkers combinations are selected to characterize the phenotype of interest and can be selected from the biomarkers and phenotypes described herein.

A microvesicle payload molecule can be assessed as a member of a biosignature panel. A payload molecule comprises any of the biological entities contained within a cell, cell fragment or vesicle membrane. These entities include without limitation nucleic acids, e.g., mRNA, microRNA, or DNA fragments; protein, e.g., soluble and membrane associated proteins; carbohydrates; lipids; metabolites; and various small molecules, e.g., hormones. The payload can be part of the cellular milieu that is encapsulated as a vesicle is formed in the cellular environment. In some embodiments of the invention, the payload is analyzed in addition to detecting vesicle surface antigens. Specific populations of vesicles can be captured as described above then the payload in the captured vesicles can be used to characterize a phenotype. For example, vesicles captured on a substrate can be further isolated to assess the payload therein. Alternately, the vesicles in a sample are detected and sorted without capture. The vesicles so detected can be further isolated to assess the payload therein. In an embodiment, vesicle populations are sorted by flow cytometry and the payload in the sorted vesicles is analyzed. In the scheme shown in FIG. 2F iv), a population of vesicles is captured and/or detected (220) using one or more of cell-of-origin biomarkers (220), disease biomarkers (221), and/or general vesicle markers (222). The payload of the isolated vesicles is assessed (223). A biosignature detected within the payload can be used to characterize a phenotype (224). In a non-limiting example, a vesicle population can be analyzed in a plasma sample from a patient using antibodies against one or more vesicle antigens of interest. The antibodies can be capture antibodies which are tethered to a substrate to isolate a desired vesicle population. Alternately, the antibodies can be directly labeled and the labeled vesicles isolated by sorting with flow cytometry. The presence or level of microRNA or mRNA extracted from the isolated vesicle population can be used to detect a biosignature. The biosignature is then used to diagnose, prognose or theranose the patient.

In other embodiments, vesicle or cellular payload is analyzed in a population (e.g., cells or vesicles) without first capturing or detected subpopulations of vesicles. For example, a cellular or extracellular vesicle population can be generally isolated from a sample using centrifugation, filtration, chromatography, or other techniques as described herein and known in the art. The payload of such sample components can be analyzed thereafter to detect a biosignature and characterize a phenotype. In the scheme shown in FIG. 2F v), a population of vesicles is isolated (230) and the payload of the isolated vesicles is assessed (231). A biosignature comprising the payload can be used to characterize a phenotype (232). In a non-limiting example, a vesicle population is isolated from a plasma sample from a patient using size exclusion and membrane filtration. The presence or level of microRNA or mRNA extracted from the vesicle population is used to detect a biosignature. The biosignature is then used to diagnose, prognose or theranose the patient.

Another illustrative scheme for characterizing a phenotype is shown in FIG. 2G vi). One or more vesicle of interest is captured and detected using a combination of cell-of-origin biomarkers (250) and disease biomarkers (251). For example, the vesicles of interest can be captured using a cell-of-origin (250) biomarker and detected using a disease-specific (251) biomarker. Similarly, the vesicles of interest can be captured using a disease-specific (251) biomarker and detected using a cell-of-origin (250) biomarker. If appropriate, the vesicle of interest can be captured and detected using only cell-of-origin (250) biomarkers or only disease-specific (251) biomarkers. In this case, the same biomarker could be used for capture and detection (e.g., anti-EpCAM capture and anti-EpCAM detector, or anti-PCSA capture and anti-PCSA detector, etc.), or different biomarkers from the same class can be used for capture and detection (e.g., anti-EpCAM capture and anti-B7H3 detector, or anti-PCSA capture and anti-PSMA detector, etc.). The phenotype can be characterized based on the detected vesicles. Optionally, payload (252) in the vesicles of interest can be assessed in order to characterize the phenotype.

The biomarkers used to detect a vesicle population can be selected to detect a microvesicle population of interest, e.g., a population of vesicles that provides a diagnosis, prognosis or theranosis of a selected condition or disease, including but not limited to a cancer, a premalignant condition, an inflammatory disease, an immune disease, an autoimmune disease or disorder, a cardiovascular disease or disorder, neurological disease or disorder, infectious disease or pain. See Section “Phenotypes” herein for more detail. In an embodiment, the biomarkers are selected from the group consisting of EpCam (epithelial cell adhesion molecule), CD9 (tetraspanin CD9 molecule), PCSA (prostate cell specific antigen, see Rokhlin et al., 5E10: a prostate-specific surface-reactive monoclonal antibody. Cancer Lett. 1998 131:129-36), CD63 (tetraspanin CD63 molecule), CD81 (tetraspanin CD81 molecule), PSMA (FOLH1, folate hydrolase (prostate-specific membrane antigen) 1), B7H3 (CD276 molecule), PSCA (prostate stem cell antigen), ICAM (intercellular adhesion molecule), STEAP (STEAP1, six transmembrane epithelial antigen of the prostate 1), KLK2 (kallikrein-related peptidase 2), SSX2 (synovial sarcoma, X breakpoint 2), SSX4 (synovial sarcoma, X breakpoint 4), PBP (prostatic binding protein), SPDEF (SAM pointed domain containing ets transcription factor), EGFR (epidermal growth factor receptor), and a combination thereof. One or more of these markers can provide a biosignature for a specific condition, such as to detect a cancer, including without limitation a carcinoma, a prostate cancer, a breast cancer, a lung cancer, a colorectal cancer, an ovarian cancer, melanoma, a brain cancer, or other type of cancer as disclosed herein. In an embodiment, a binding agent to one or more of these markers is used to capture a microvesicle population, and an aptamer of the invention is used to assist in detection of the capture vesicles as described herein. In other embodiments, an aptamer of the invention is used to capture a microvesicle population, and a binding agent to one or more of these markers is used to assist in detection of the capture vesicles as described herein. The binding agents can be any useful binding agent as disclosed herein or known in the art, e.g., antibodies or aptamers.

The invention also contemplates use of a lipid dye to stain a microvesicle population. For example, a lipid dye can allow a vesicle to be visualized using flow cytometry, microparticle assay, immunoassay, or other technologies that can detect the dye. The lipid dye can be used instead of or in addition to using a detector binding agent. For example, a lipid dye can be used to stain an entire microvesicle population that can then be captured using a binding agent as described herein. The captured vesicle can be detected by detecting the lipid dye. Alternately, the microvesicle population can also be detected using a labeled binding agent as a detection agent. The vesicle population could then be detected using either or both of the lipid dye and the detection agent. In an aspect method of detecting a presence or level of one or more microvesicle in a biological sample, comprising: a) contacting a biological sample with a lipid staining dye, wherein the biological sample comprises or is suspected to comprise the one or more microvesicle; and b) detecting the lipid staining dye in contact with the one or more microvesicle, thereby detecting the presence or level of the one or more microvesicle.

The invention can make use of any appropriate dye that can be associated with a vesicle membrane. For example, the dye may comprise a hydrophobic chain and a detectable moiety. In various embodiments of the method, the lipid staining dye comprises a long-chain dialkylcarbocyanine, an indocarbocyanine (DiI), an oxacarbocyanine (DiO), FM 1-43, FM 1-43FX, FM 4-64, FM 5-95, a dialkyl aminostyryl dye, DiA, a long-wavelength light-excitable carbocyanines (DiD), an infrared light-excitable carbocyanine (DiR), carboxyfluorescein succinimidyl ester (CFDA), carboxyfluorescein succinimidyl ester (CFSE), 4-(4-(Dihexadecylamino)styryl)-N-Methylpyridinium Iodide (DiA; 4-Di-16-ASP), 4-(4-(Didecylamino)styryl)-N-Methylpyridinium Iodide (4-Di-10-ASP), 1,1′-Dioctadecyl-3,3,3′,3′-Tetramethylindodicarbocyanine Perchlorate (‘DiD’ oil; DiIC₁₈(5) oil), E1′-Dioctadecyl-3,3,3′,3′-Tetramethylindodicarbocyanine, 4-Chlorobenzenesulfonate Salt (‘DiD’ solid; DiIC₁₈(5) solid), 1,1′-Dioleyl-3,3,3′,3′-Tetramethylindocarbocyanine methanesulfonate (Δ⁹-DiI), Dil Stain (1,1′-Dioctadecyl-3,3,3′,3′-Tetramethylindocarbocyanine Perchlorate (‘DiI’; DiIC₁₈(3))), Dil Stain (1,1′-Dioctadecyl-3,3,3′,3′-Tetramethylindocarbocyanine Perchlorate (‘DiI’; DiIC₁₈(3))), 1,1′-Didodecyl-3,3,3′,3′-Tetramethylindocarbocyanine Perchlorate (DiIC₁₂(3)), 1,1′-Dihexadecyl-3,3,3′,3′-Tetramethylindocarbocyanine Perchlorate (DiIC₁₆(3)), 1,1′-Dioctadecyl-3,3,3′,3′-Tetramethylindocarbocyanine-5,5′-Disulfonic Acid (DiIC₁₈(3)-DS), 1,1′-Dioctadecyl-3,3,3′,3′-Tetramethylindodicarbocyanine-5,5′-Disulfonic Acid (DiIC₁₈(5)-DS), 4-(4-(Dilinoleylamino)styryl)-N-Methylpyridinium 4-Chlorobenzenesulfonate (FAST DiA™ solid; DiΔ^{9,12}-C₁₈ASP, CBS), 3,3′-Dilinoleyloxacarbocyanine Perchlorate (FAST DiO™ Solid; DiOΔ^{9,12}-C₁₈(3), ClO₄), 1,1′-Dilinoleyl-3,3,3′,3′-Tetramethylindocarbocyanine, 4-Chlorobenzenesulfonate (FAST DiI™ solid; DiIA^{9,12}-C₁₈(3), CBS), 1,1′-Dilinoleyl-3,3,3′,3′-Tetramethylindocarbocyanine Perchlorate (FAST DiI™ oil; DiIA^{9,12}-C₁₈(3), ClO₄), 3,3′-Dioctadecyloxacarbocyanine Perchlorate (‘DiO’; DiOC₁₈(3)), 3,3′-Dihexadecyloxacarbocyanine Perchlorate (DiOC₁₆(3)), 3,3′-Dioctadecyl-5,5′-Di(4-Sulfophenyl)Oxacarbocyanine, Sodium Salt (SP-DiOC₁₈(3)), 1,1′-Dioctadecyl-6,6′-Di(4-Sulfophenyl)-3,3,3′,3′-Tetramethylindocarbocyanine (SP-DiIC₁₈(3)), 1,1′-Dioctadecyl-3,3,3′,3′-Tetramethylindotricarbocyanine Iodide (DiR; DiIC₁₈(7)), 3,3′-Diethylthiacarbocyanine iodide, 3,3′-Diheptylthiacarbocyanine iodide, 3,3′-Dioctylthiacarbocyanine iodide, 3,3′-Dipropylthiadicarbocyanine iodide, 7-(Diethylamino)coumarin-3-carboxylic acid, 7-(Diethylamino)coumarin-3-carboxylic acid N-succinimidyl ester, an analog or variant of any thereof, and a combination of any thereof.

In some embodiments, the lipid staining dye is labeled. The label can be an activatable label. For example, the lipid staining dye may be converted from a non-labeled form to a labeled form upon contact with the microvesicle, thereby decreasing background from non-bound dye. Such a dye can comprise an esterase-activated lipophilic dye. As a non-limiting example, the microvesicles can be contacted with a carboxyfluorescein succinimidyl ester (CFDA) dye. Microvesicle associated esterases will convert the CFDA to carboxyfluorescein succinimidyl ester (CFSE), which can be detected using a fluorescence reader. See Example 48 herein for further details.

The method of staining a microvesicle population with a lipid staining dye may comprise detecting a level of one or more microvesicle in a series of biological samples having known microvesicle concentrations; and constructing a standard curve from the detected levels. The standard curve can be used to calculate a microvesicle concentration in a test sample. For example, a detected level of one or more microvesicle in a test sample can be interpolated to a standard curve, thereby determining the microvesicle concentration in the test sample. See Examples 47-48 herein for further details.

An illustrative scheme for detecting microvesicles and/or characterizing a phenotype using a lipid dye is shown in FIG. 2H vii). A biological sample is provided which comprises or is suspected to comprise one or more vesicle of interest. As shown, the population can be directly contact with lipid dye (260) prior to capture and/or detection using one or more of a cell-of-origin biomarker (261), e.g., as in Table 4 or 5, disease biomarkers (262), e.g., as in Table 4 or 5, and general vesicle marker (263), e.g., as in Table 3. For example, the vesicles of interest can be captured using a cell-of-origin (261) biomarker and detected using a disease-specific (262) biomarker. Similarly, the vesicles of interest can be captured using a disease-specific (262) biomarker and detected using a cell-of-origin (261) biomarker. If appropriate, the vesicle of interest can be captured and detected using only cell-of-origin (261) biomarkers or only disease-specific (262) biomarkers. The vesicles can also be captured and/or detected using one or more general vesicle marker (263). In this case, the same biomarker could be used for capture and detection (e.g., anti-EpCAM capture and anti-EpCAM detector, or anti-PCSA capture and anti-PCSA detector, etc.), or different biomarkers from the same class can be used for capture and detection (e.g., anti-EpCAM capture and anti-B7H3 detector, or anti-PCSA capture and anti-PSMA detector, etc.). The captured and/or detected microvesicles can also be contacted with lipid dye (265). In some embodiments, capture is performed using a binding agent to a specific biomarker as described above then the vesicles are detected only using lipid dye (265). The phenotype can be characterized based on the detected vesicles. Optionally, payload in the vesicles of interest can be assessed in order to characterize the phenotype.

The methods of characterizing a phenotype can employ a combination of techniques to assess a vesicle population in a sample of interest. In an embodiment, the sample is split into various aliquots and each is analyzed separately. For example, protein content of one or more aliquot is determined and microRNA content of one or more other aliquot is determined. The protein content and microRNA content can be combined to characterize a phenotype. In another embodiment, vesicles of interest are isolated and the payload therein is assessed. For example, a population of vesicles with a given surface marker can be isolated by affinity isolation such as flow cytometry, immunoprecipitation, or other immunocapture technique using a binding agent to the surface marker of interest. The isolated vesicles can then be assessed for biomarkers such as surface content or payload. The biomarker profile of vesicles having the given surface marker can be used to characterize a phenotype. As a non-limiting example, a PCSA+ capture agent can be used to isolate a prostate specific vesicle population. Levels of surface antigens such as PCSA itself, PSMA, B7H3, or EpCam can be assessed from the PCSA+ vesicles. Levels of payload in the PCSA+ can also be assessed, e.g., microRNA or mRNA content. A biosignature can be constructed from a combination of the markers in the PCSA+ vesicle population.

In an embodiment, the invention provides a method of isolating a microvesicle population and assessing the microRNA with the isolated microvesicles. The microvesicle can be bound in a microtiter plate well that has been coated with a binding agent to a general vesicle biomarker, a cell-of-origin vesicle biomarker, or a disease-specific vesicle biomarker. As desired, vesicles in the wells can be detected using one or more detector agent to a general vesicle biomarker, a cell-of-origin vesicle biomarker, or a disease-specific vesicle biomarker. RNA can be isolated from microvesicles in wells that comprise the vesicles of interest. MicroRNA or miRNA content derived from the microvesicles are then detected. The presence or levels of the vesicle markers and RNA markers can be used to construct a biosignature as described herein. The biosignature can be used to characterize a phenotype of interest.

In another embodiment, contaminants are removed from a biological sample and the remaining vesicles are assessed for surface content and/or payload. For example, a column can be constructed comprising binding agents to contaminating proteins, vesicles, or other entities in the biological sample. The flow through will thereby be enriched in the circulating biomarkers or circulating microvesicles of interest. In a non-limiting example, a column is constructed to remove microvesicles derived from blood cells. The column can be used to enrich microvesicles in a blood sample that are derived from non-blood cell origin. The enrichment scheme can be used to remove protein aggregates, nucleic acids in solution, etc. One of skill will appreciate that this enrichment can be used with other vesicle or biomarkers methodology presented herein to assess vesicle or biomarkers or interest. To continue the non-limiting example, the flow through that has been depleted in vesicles from blood cells can then be analyzed via a positive selection for vesicles of interest using affinity techniques or the like.

A peptide or protein biomarker can be analyzed by mass spectrometry or flow cytometry. Proteomic analysis of a vesicle may be carried out by immunocytochemical staining, Western blotting, electrophoresis, SDS-PAGE, chromatography, x-ray crystallography or other protein analysis techniques in accordance with procedures well known in the art. In other embodiments, the protein biosignature of a vesicle may be analyzed using 2 D differential gel electrophoresis as described in, Chromy et al. J Proteome Res, 2004; 3:1120-1127, which is herein incorporated by reference in its entirety, or with liquid chromatography mass spectrometry as described in Zhang et al. Mol Cell Proteomics, 2005; 4:144-155, which is herein incorporated by reference in its entirety. A vesicle may be subjected to activity-based protein profiling described for example, in Berger et al., Am J Pharmacogenomics, 2004; 4:371-381, which is in incorporated by reference in its entirety. In other embodiments, a vesicle may be profiled using nanospray liquid chromatography-tandem mass spectrometry as described in Pisitkun et al., Proc Natl Acad Sci US A, 2004; 101:13368-13373, which is herein incorporated by reference in its entirety. In another embodiment, the vesicle may be profiled using tandem mass spectrometry (MS) such as liquid chromatography/MS/MS (LC-MS/MS) using for example a LTQ and LTQ-FT ion trap mass spectrometer. Protein identification can be determined and relative quantitation can be assessed by comparing spectral counts as described in Smalley et al., J Proteome Res, 2008; 7:2088-2096, which is herein incorporated by reference in its entirety.

The expression of circulating protein biomarkers or protein payload within a vesicle can also be identified. The latter analysis can optionally follow the isolation of specific vesicles using capture agents to capture populations of interest. In an embodiment, immunocytochemical staining is used to analyze protein expression. The sample can be resuspended in buffer, centrifuged at 100×g for example, for 3 minutes using a cytocentrifuge on adhesive slides in preparation for immunocytochemical staining. The cytospins can be air-dried overnight and stored at −80° C. until staining. Slides can then be fixed and blocked with serum-free blocking reagent. The slides can then be incubated with a specific antibody to detect the expression of a protein of interest. In some embodiments, the vesicles are not purified, isolated or concentrated prior to protein expression analysis.

Biosignatures comprising vesicle payload can be characterized by analysis of a metabolite marker or metabolite within the vesicle. Various metabolite-oriented approaches have been described such as metabolite target analyses, metabolite profiling, or metabolic fingerprinting, see for example, Denkert et al., Molecular Cancer 2008; 7: 4598-4617, Ellis et al., Analyst 2006; 8: 875-885, Kuhn et al., Clinical Cancer Research 2007; 24: 7401-7406, Fiehn O., Comp Funct Genomics 2001; 2:155-168, Fancy et al., Rapid Commun Mass Spectrom 20(15): 2271-80 (2006), Lindon et al., Pharm Res, 23(6): 1075-88 (2006), Holmes et al., Anal Chem. 2007 Apr. 1; 79(7):2629-40. Epub 2007 Feb. 27. Erratum in: Anal Chem. 2008 Aug. 1; 80(15):6142-3, Stanley et al., Anal Biochem. 2005 Aug. 15; 343(2): 195-202., Lehtimäki et al., J Biol Chem. 2003 Nov. 14; 278(46):45915-23, each of which is herein incorporated by reference in its entirety.

Peptides can be analyzed by systems described in Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety. This system can generate sensitive molecular fingerprints of proteins present in a body fluid as well as in vesicles. Commercial applications which include the use of chromatography/mass spectroscopy and reference libraries of all stable metabolites in the human body, for example Paradigm Genetic's Human Metabolome Project, may be used to determine a metabolite biosignature. Other methods for analyzing a metabolic profile can include methods and devices described in U.S. Pat. No. 6,683,455 (Metabometrix), U.S. Patent Application Publication Nos. 20070003965 and 20070004044 (Biocrates Life Science), each of which is herein incorporated by reference in its entirety. Other proteomic profiling techniques are described in Kennedy, Toxicol Lett 120:379-384 (2001), Berven et al., Curr Pharm Biotechnol 7(3): 147-58 (2006), Conrads et al., Expert Rev Proteomics 2(5): 693-703, Decramer et al., World J Urol 25(5): 457-65 (2007), Decramer et al., Mol Cell Proteomics 7(10): 1850-62 (2008), Decramer et al., Contrib Nephrol, 160: 127-41 (2008), Diamandis, J Proteome Res 5(9): 2079-82 (2006), Immler et al., Proteomics 6(10): 2947-58 (2006), Khan et al., J Proteome Res 5(10): 2824-38 (2006), Kumar et al., Biomarkers 11(5): 385-405 (2006), Noble et al., Breast Cancer Res Treat 104(2): 191-6 (2007), Omenn, Dis Markers 20(3): 131-4 (2004), Powell et al., Expert Rev Proteomics 3(1): 63-74 (2006), Rai et al., Arch Pathol Lab Med, 126(12): 1518-26 (2002), Ramstrom et al., Proteomics, 3(2): 184-90 (2003), Tammen et al., Breast Cancer Res Treat, 79(1): 83-93 (2003), Theodorescu et al., Lancet Oncol, 7(3): 230-40 (2006), or Zurbig et al., Electrophoresis, 27(11): 2111-25 (2006).

For analysis of mRNAs, miRNAs or other small RNAs, the total RNA can be isolated using any known methods for isolating nucleic acids such as methods described in U.S. Patent Application Publication No. 2008132694, which is herein incorporated by reference in its entirety. These include, but are not limited to, kits for performing membrane based RNA purification, which are commercially available. Generally, kits are available for the small-scale (30 mg or less) preparation of RNA from cells and tissues, for the medium scale (250 mg tissue) preparation of RNA from cells and tissues, and for the large scale (1 g maximum) preparation of RNA from cells and tissues. Other commercially available kits for effective isolation of small RNA-containing total RNA are available. Such methods can be used to isolate nucleic acids from vesicles.

Alternatively, RNA can be isolated using the method described in U.S. Pat. No. 7,267,950, which is herein incorporated by reference in its entirety. U.S. Pat. No. 7,267,950 describes a method of extracting RNA from biological systems (cells, cell fragments, organelles, tissues, organs, or organisms) in which a solution containing RNA is contacted with a substrate to which RNA can bind and RNA is withdrawn from the substrate by applying negative pressure. Alternatively, RNA may be isolated using the method described in U.S. Patent Application No. 20050059024, which is herein incorporated by reference in its entirety, which describes the isolation of small RNA molecules. Other methods are described in U.S. Patent Application No. 20050208510, 20050277121, 20070238118, each of which is incorporated by reference in its entirety.

In one embodiment, mRNA expression analysis can be carried out on mRNAs from a vesicle isolated from a sample. In some embodiments, the vesicle is a cell-of-origin specific vesicle. An expression pattern generated from a vesicle can be indicative of a given disease state, disease stage, therapy related signature, or physiological condition.

In one embodiment, once the total RNA has been isolated, cDNA can be synthesized and either qRT-PCR assays (e.g. Applied Biosystem's Taqman® assays) for specific mRNA targets can be performed according to manufacturer's protocol, or an expression microarray can be performed to look at highly multiplexed sets of expression markers in one experiment. Methods for establishing gene expression profiles include determining the amount of RNA that is produced by a gene that can code for a protein or peptide. This can be accomplished by quantitative reverse transcriptase PCR (qRT-PCR), competitive RT-PCR, real time RT-PCR, differential display RT-PCR, Northern Blot analysis or other related tests. While it is possible to conduct these techniques using individual PCR reactions, it is also possible to amplify complementary DNA (cDNA) or complementary RNA (cRNA) produced from mRNA and analyze it via microarray.

The level of a miRNA product in a sample can be measured using any appropriate technique that is suitable for detecting mRNA expression levels in a biological sample, including but not limited to Northern blot analysis, RT-PCR, qRT-PCR, in situ hybridization or microarray analysis. For example, using gene specific primers and target cDNA, qRT-PCR enables sensitive and quantitative miRNA measurements of either a small number of target miRNAs (via singleplex and multiplex analysis) or the platform can be adopted to conduct high throughput measurements using 96-well or 384-well plate formats. See for example, Ross J S et al, Oncologist. 2008 May; 13(5):477-93, which is herein incorporated by reference in its entirety. A number of different array configurations and methods for microarray production are known to those of skill in the art and are described in U.S. patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; or 5,700,637; each of which is herein incorporated by reference in its entirety. Other methods of profiling miRNAs are described in Taylor et al., Gynecol Oncol. 2008 July; 110(1): 13-21, Gilad et al, PLoS ONE. 2008 Sep. 5; 3(9):e3148, Lee et al., Annu Rev Pathol. 2008 Sep. 25 and Mitchell et al, Proc Natl Acad Sci USA. 2008 Jul. 29; 105(30):10513-8, Shen R et al, BMC Genomics. 2004 Dec. 14; 5(1):94, Mina L et al, Breast Cancer Res Treat. 2007 June; 103(2):197-208, Zhang L et al, Proc Natl Acad Sci USA. 2008 May 13; 105(19):7004-9, Ross J S et al, Oncologist. 2008 May; 13(5):477-93, Schetter A J et al, JAMA. 2008 Jan. 30; 299(4):425-36, Staudt L M, N Engl J Med 2003; 348:1777-85, Mulligan G et al, Blood. 2007 Apr. 15; 109(8):3177-88. Epub 2006 Dec. 21, McLendon R et al, Nature. 2008 Oct. 23; 455(7216):1061-8, and U.S. Pat. Nos. 5,538,848, 5,723,591, 5,876,930, 6,030,787, 6,258,569, and 5,804,375, each of which is herein incorporated by reference. In some embodiments, arrays of microRNA panels are use to simultaneously query the expression of multiple miRs. The Exiqon mIRCURY LNA microRNA PCR system panel (Exiqon, Inc., Woburn, Mass.) or the TaqMan® MicroRNA Assays and Arrays systems from Applied Biosystems (Foster City, Calif.) can be used for such purposes.

Microarray technology allows for the measurement of the steady-state mRNA or miRNA levels of thousands of transcripts or miRNAs simultaneously thereby presenting a powerful tool for identifying effects such as the onset, arrest, or modulation of uncontrolled cell proliferation. Two microarray technologies, such as cDNA arrays and oligonucleotide arrays can be used. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA or miRNA, expressed in the sample cells. A large number of such techniques are available and useful. Methods for determining gene expression can be found in U.S. Pat. No. 6,271,002 to Linsley, et al.; U.S. Pat. No. 6,218,122 to Friend, et al.; U.S. Pat. No. 6,218,114 to Peck et al.; or U.S. Pat. No. 6,004,755 to Wang, et al., each of which is herein incorporated by reference in its entirety.

Analysis of an expression level can be conducted by comparing such intensities. This can be performed by generating a ratio matrix of the expression intensities of genes in a test sample versus those in a control sample. The control sample may be used as a reference, and different references to account for age, ethnicity and sex may be used. Different references can be used for different conditions or diseases, as well as different stages of diseases or conditions, as well as for determining therapeutic efficacy.

For instance, the gene expression intensities of mRNA or miRNAs derived from a diseased tissue, including those isolated from vesicles, can be compared with the expression intensities of the same entities in normal tissue of the same type (e.g., diseased breast tissue sample versus normal breast tissue sample). A ratio of these expression intensities indicates the fold-change in gene expression between the test and control samples. Alternatively, if vesicles are not normally present in from normal tissues (e.g. breast) then absolute quantitation methods, as is known in the art, can be used to define the number of miRNA molecules present without the requirement of miRNA or mRNA isolated from vesicles derived from normal tissue.

Gene expression profiles can also be displayed in a number of ways. A common method is to arrange raw fluorescence intensities or ratio matrix into a graphical dendogram where columns indicate test samples and rows indicate genes. The data is arranged so genes that have similar expression profiles are proximal to each other. The expression ratio for each gene is visualized as a color. For example, a ratio less than one (indicating down-regulation) may appear in the blue portion of the spectrum while a ratio greater than one (indicating upregulation) may appear as a color in the red portion of the spectrum. Commercially available computer software programs are available to display such data.

mRNAs or miRNAs that are considered differentially expressed can be either over expressed or under expressed in patients with a disease relative to disease free individuals. Over and under expression are relative terms meaning that a detectable difference (beyond the contribution of noise in the system used to measure it) is found in the amount of expression of the mRNAs or miRNAs relative to some baseline. In this case, the baseline is the measured mRNA/miRNA expression of a non-diseased individual. The mRNA/miRNA of interest in the diseased cells can then be either over or under expressed relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis includes the determination of disease/status issues such as determining the likelihood of relapse or metastasis and therapy monitoring. In therapy monitoring, clinical judgments are made regarding the effect of a given course of therapy by comparing the expression of genes over time to determine whether the mRNA/miRNA expression profiles have changed or are changing to patterns more consistent with normal tissue.

Levels of over and under expression are distinguished based on fold changes of the intensity measurements of hybridized microarray probes. A 2X difference is preferred for making such distinctions or a p-value less than 0.05. That is, before an mRNA/miRNA is the to be differentially expressed in diseased/relapsing versus normal/non-relapsing cells, the diseased cell is found to yield at least 2 times more, or 2 times less intensity than the normal cells. The greater the fold difference, the more preferred is use of the gene as a diagnostic or prognostic tool. mRNA/miRNAs selected for the expression profiles of the instant invention have expression levels that result in the generation of a signal that is distinguishable from those of the normal or non-modulated genes by an amount that exceeds background using clinical laboratory instrumentation.

Statistical values can be used to confidently distinguish modulated from non-modulated mRNA/miRNA and noise. Statistical tests find the mRNA/miRNA most significantly different between diverse groups of samples. The Student's t-test is an example of a robust statistical test that can be used to find significant differences between two groups. The lower the p-value, the more compelling the evidence that the gene shows a difference between the different groups. Nevertheless, since microarrays measure more than one mRNA/miRNA at a time, tens of thousands of statistical tests may be performed at one time. Because of this, one is unlikely to see small p-values just by chance and adjustments for this using a Sidak correction as well as a randomization/permutation experiment can be made. A p-value less than 0.05 by the t-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

In one embodiment, a method of generating a posterior probability score to enable diagnostic, prognostic, therapy-related, or physiological state specific biosignature scores can be arrived at by obtaining circulating biomarker expression data from a statistically significant number of patients; applying linear discrimination analysis to the data to obtain selected biomarkers; and applying weighted expression levels to the selected biomarkers with discriminate function factor to obtain a prediction model that can be applied as a posterior probability score. Other analytical tools can also be used to answer the same question such as, logistic regression and neural network approaches.

For instance, the following can be used for linear discriminant analysis:

where,
I(pₛi_{d})=The log base 2 intensity of the probe set enclosed in parenthesis. d(cp)=The discriminant function for the disease positive class d(C_{N})=The discriminant function for the disease negative class
P(_{CP})=The posterior p-value for the disease positive class
P(_{CN})=The posterior p-value for the disease negative class

Numerous other well-known methods of pattern recognition are available. The following references provide some examples: Weighted Voting: Golub et al. (1999); Support Vector Machines: Su et al. (2001); and Ramaswamy et al. (2001); K-nearest Neighbors: Ramaswamy (2001); and Correlation Coefficients: van't Veer et al. (2002), all of which are herein incorporated by reference in their entireties.

A biosignature portfolio, further described below, can be established such that the combination of biomarkers in the portfolio exhibit improved sensitivity and specificity relative to individual biomarkers or randomly selected combinations of biomarkers. In one embodiment, the sensitivity of the biosignature portfolio can be reflected in the fold differences, for example, exhibited by a transcript's expression in the diseased state relative to the normal state. Specificity can be reflected in statistical measurements of the correlation of the signaling of transcript expression with the condition of interest. For example, standard deviation can be a used as such a measurement. In considering a group of biomarkers for inclusion in a biosignature portfolio, a small standard deviation in expression measurements correlates with greater specificity. Other measurements of variation such as correlation coefficients can also be used in this capacity.

Another parameter that can be used to select mRNA/miRNA that generate a signal that is greater than that of the non-modulated mRNA/miRNA or noise is the use of a measurement of absolute signal difference. The signal generated by the modulated mRNA/miRNA expression is at least 20% different than those of the normal or non-modulated gene (on an absolute basis). It is even more preferred that such mRNA/miRNA produce expression patterns that are at least 30% different than those of normal or non-modulated mRNA/miRNA.

MiRNA can also be detected and measured by amplification from a biological sample and measured using methods described in U.S. Pat. No. 7,250,496, U.S. Application Publication Nos. 20070292878, 20070042380 or 20050222399 and references cited therein, each of which is herein incorporated by reference in its entirety. The microRNA can be assessed as in U.S. Pat. No. 7,888,035, entitled “METHODS FOR ASSESSING RNA PATTERNS,” issued Feb. 15, 2011, which application is incorporated by reference herein in its entirety.

The levels of microRNA can be normalized using various techniques known to those of skill in the art. For example, relative quantification of miRNA expression can be performed using the 2^{-ΔΔCT} method (Applied Biosystems User Bulletin N^{o}2). The levels of microRNA can also be normalized to housekeeping nucleic acids, such as housekeeping mRNAs, microRNA or snoRNA. Further methods for normalizing miRNA levels that can be used with the invention are described further in Vasilescu, MicroRNA fingerprints identify miR-150 as a plasma prognostic marker in patients with sepsis. PLoS One. 2009 Oct. 12; 4(10):e7405; and Peltier and Latham, Normalization of microRNA expression levels in quantitative RT-PCR assays: identification of suitable reference RNA targets in normal and cancerous human solid tissues. RNA. 2008 May; 14(5):844-52. Epub 2008 Mar. 28; each of which reference is herein incorporated by reference in its entirety.

Peptide nucleic acids (PNAs) which are a new class of synthetic nucleic acid analogs in which the phosphate—sugar polynucleotide backbone is replaced by a flexible pseudo-peptide polymer may be used in analysis of a biosignature. PNAs are capable of hybridizing with high affinity and specificity to complementary RNA and DNA sequences and are highly resistant to degradation by nucleases and proteinases. Peptide nucleic acids (PNAs) are an attractive new class of probes with applications in cytogenetics for the rapid in situ identification of human chromosomes and the detection of copy number variation (CNV). Multicolor peptide nucleic acid-fluorescence in situ hybridization (PNA-FISH) protocols have been described for the identification of several human CNV-related disorders and infectious diseases. PNAs can also be used as molecular diagnostic tools to non-invasively measure oncogene mRNAs with tumor targeted radionuclide-PNA-peptide chimeras. Methods of using PNAs are described further in Pellestor F et al, Curr Pharm Des. 2008; 14(24):2439-44, Tian X et al, Ann N Y Acad Sci. 2005 November; 1059:106-44, Paulasova P and Pellestor F, Annales de Genetique, 47 (2004) 349-358, Stender H. Expert Rev Mol Diagn. 2003 Sep. 3(5):649-55. Review, Vigneault et al., Nature Methods, 5(9), 777-779 (2008), each reference is herein incorporated by reference in its entirety. These methods can be used to screen the genetic materials isolated from a vesicle. When applying these techniques to a cell-of-origin specific vesicle, they can be used to identify a given molecular signal that directly pertains to the cell of origin.

Mutational analysis may be carried out for mRNAs and DNA, including those that are identified from a vesicle. For mutational analysis of a target or biomarker that is of RNA origin, the RNA (mRNA, miRNA or other) can be reverse transcribed into cDNA and subsequently sequenced or assayed, such as for known SNPs (by Taqman SNP assays, for example) or single nucleotide mutations, as well as using sequencing to look for insertions or deletions to determine mutations present in the cell-of-origin. Multiplexed ligation dependent probe amplification (MLPA) could alternatively be used for the purpose of identifying CNV in small and specific areas of interest. For example, once the total RNA has been obtained from isolated colon cancer-specific vesicles, cDNA can be synthesized and primers specific for exons 2 and 3 of the KRAS gene can be used to amplify these two exons containing codons 12, 13 and 61 of the KRAS gene. The same primers used for PCR amplification can be used for Big Dye Terminator sequence analysis on the ABI 3730 to identify mutations in exons 2 and 3 of KRAS. Mutations in these codons are known to confer resistance to drugs such as Cetuximab and Panitumimab. Methods of conducting mutational analysis are described in Maheswaran S et al, Jul. 2, 2008 (10.1056/NEJMoa0800668) and Orita, M et al, PNAS 1989, (86): 2766-70, each of which is herein incorporated by reference in its entirety.

Other methods of conducting mutational analysis include miRNA sequencing. Applications for identifying and profiling miRNAs can be done by cloning techniques and the use of capillary DNA sequencing or “next-generation” sequencing technologies. The new sequencing technologies currently available allow the identification of low-abundance miRNAs or those exhibiting modest expression differences between samples, which may not be detected by hybridization-based methods. Such new sequencing technologies include the massively parallel signature sequencing (MPSS) methodology described in Nakano et al. 2006, Nucleic Acids Res. 2006; 34:D731-D735. doi: 10.1093/nar/gkj077, the Roche/454 platform described in Margulies et al. 2005, Nature. 2005; 437:376-380 or the Illumina sequencing platform described in Berezikov et al. Nat. Genet. 2006b; 38:1375-1377, each of which is incorporated by reference in its entirety.

Additional methods to determine a biosignature includes assaying a biomarker by allele-specific PCR, which includes specific primers to amplify and discriminate between two alleles of a gene simultaneously, single-strand conformation polymorphism (SSCP), which involves the electrophoretic separation of single-stranded nucleic acids based on subtle differences in sequence, and DNA and RNA aptamers. DNA and RNA aptamers are short oligonucleotide sequences that can be selected from random pools based on their ability to bind a particular molecule with high affinity. Methods of using aptamers are described in Ulrich H et al, Comb Chem High Throughput Screen. 2006 Sep. 9(8):619-32, Ferreira C S et al, Anal Bioanal Chem. 2008 February; 390(4):1039-50, Ferreira C S et al, Tumour Biol. 2006; 27(6):289-301, each of which is herein incorporated by reference in its entirety.

Biomarkers can also be detected using fluorescence in situ hybridization (FISH). Methods of using FISH to detect and localize specific DNA sequences, localize specific mRNAs within tissue samples or identify chromosomal abnormalities are described in Shaffer D R et al, Clin Cancer Res. 2007 Apr. 1; 13(7):2023-9, Cappuzo F et al, Journal of Thoracic Oncology, Volume 2, Number 5, May 2007, Moroni M et al, Lancet Oncol. 2005 May; 6(5):279-86, each of which is herein incorporated by reference in its entirety.

An illustrative schematic for analyzing a population of vesicles for their payload is presented in FIG. 2F. In the embodiment in FIG. 2F v), the methods of the invention include characterizing a phenotype by isolating vesicles (230) and determining a level of microRNA species contained therein (231), thereby characterizing the phenotype (232).

A biosignature comprising a circulating biomarker or vesicle can comprise a binding agent thereto. The binding agent can be a DNA, RNA, aptamer, monoclonal antibody, polyclonal antibody, Fabs, Fab′, single chain antibody, synthetic antibody, aptamer (DNA/RNA), peptoid, zDNA, peptide nucleic acid (PNA), locked nucleic acid (LNA), lectin, synthetic or naturally occurring chemical compounds (including but not limited to drugs and labeling reagents).

A binding agent can used to isolate or detect a vesicle by binding to a component of the vesicle, as described above. The binding agent can be used to detect a vesicle, such as for detecting a cell-of-origin specific vesicle. A binding agent or multiple binding agents can themselves form a binding agent profile that provides a biosignature for a vesicle. For example, if a vesicle population is detected or isolated using two, three, four or more binding agents in a differential detection or isolation of a vesicle from a heterogeneous population of vesicles, the particular binding agent profile for the vesicle population provides a biosignature for the particular vesicle population.

As an illustrative example, a vesicle for characterizing a cancer can be detected with one or more binding agents including, but not limited to, PSA, PSMA, PCSA, PSCA, B7H3, EpCam, TMPRSS2, mAB 5D4, XPSM-A9, XPSM-A10, Galectin-3, E-selectin, Galectin-1, or E4 (IgG2a kappa), or any combination thereof.

The binding agent can also be for a general vesicle biomarker, such as a “housekeeping protein” or antigen. The biomarker can be CD9, CD63, or CD81. For example, the binding agent can be an antibody for CD9, CD63, or CD81. The binding agent can also be for other proteins, such as for tissue specific or cancer specific vesicles. The binding agent can be for PCSA, PSMA, EpCam, B7H3, or STEAP. The binding agent can be for DR3, STEAP, epha2, TMEM211, MFG-E8, Annexin V, TF, unc93A, A33, CD24, NGAL, EpCam, MUC17, TROP2, or TETS. For example, the binding agent can be an antibody or aptamer for PCSA, PSMA, EpCam, B7H3, DR3, STEAP, epha2, TMEM211, MFG-E8, Annexin V, TF, unc93A, A33, CD24, NGAL, EpCam, MUC17, TROP2, or TETS.

Various proteins are not typically distributed evenly or uniformly on a vesicle shell. Vesicle-specific proteins are typically more common, while cancer-specific proteins are less common. In some embodiments, capture of a vesicle is accomplished using a more common, less cancer-specific protein, such as one or more housekeeping proteins or antigen or general vesicle antigen (e.g., a tetraspanin), and one or more cancer-specific biomarkers and/or one or more cell-of-origin specific biomarkers is used in the detection phase. In another embodiment, one or more cancer-specific biomarkers and/or one or more cell-of-origin specific biomarkers are used for capture, and one or more housekeeping proteins or antigen or general vesicle antigen (e.g., a tetraspanin) is used for detection. In embodiments, the same biomarker is used for both capture and detection. Different binding agents for the same biomarker can be used, such as antibodies or aptamers that bind different epitopes of an antigen.

Additional cellular binding partners or binding agents may be identified by any conventional methods known in the art, or as described herein, and may additionally be used as a diagnostic, prognostic or therapy-related marker. For example, vesicles can be detected using one or more binding agent listed in Tables 3, 4 or 5 herein. For example, the binding agent can also be for a general vesicle biomarker, such as a “housekeeping protein” or antigen. The general vesicle biomarker can be CD9, CD63, or CD81, or other biomarker in Table 3. The binding agent can also be for other proteins, such as for cell of origin specific or cancer specific vesicles. As a non-limiting example, in the case of prostate cancer, the binding agent can be for PCSA, PSMA, EpCam, B7H3, RAGE or STEAP. The binding agent can be for a biomarker in Tables 4-5. For example, the binding agent can be an antibody or aptamer for PCSA, PSMA, EpCam, B7H3, RAGE, STEAP or other biomarker in Tables 4-5.

Various proteins may not be distributed evenly or uniformly on a vesicle surface. For example, vesicle-specific proteins are typically more common, while cancer-specific proteins are less common. In some embodiments, capture of a vesicle is accomplished using a more common, less cancer-specific protein, such as a housekeeping protein or antigen, and cancer-specific proteins is used in the detection phase. Depending on the sensitivity of the detection system, the opposite method can also be used wherein a large vesicle population is captured using a binding agent to a general vesicle marker and then cell-specific vesicles are detected with detection agents specific to a sub-population of interest.

Furthermore, additional cellular binding partners or binding agents may be identified by any conventional methods known in the art, or as described herein, and may additionally be used as a diagnostic, prognostic or therapy-related marker.

microRNA Functional Assay

As described above, microRNAs can be found circulating in bodily fluids such as blood encapsulated in microvesicles, HDL and LDL particles as well as components of ribonucleoprotein complexes (RNPs). microRNA can be detected using available technologies such as described herein or known in the art, including without limitation RT-qPCR or next generation sequencing. However, microRNA in a biologically active state is bound and activated by one or more of the Argonaute (“Ago”) proteins (e.g., Ago1, Ago2, Ago3, or Ago4). One aspect of the invention is directed to compositions and methods that enable detection of a functional activity of a target microRNA within a biological sample in a single reaction. For a review of the Ago family of proteins, see, Hock and Meister, Genome Biology, 2008, 9:210.

More particularly, a substrate, a synthetic RNA molecule, a label and RISC (RNA-Induced Silencing Complex) reaction buffer components, and optionally one or more isolated Ago protein, are used to assess one or more nucleic acid biomarkers (e.g., microRNAs). Examples of a substrate that can be used in the invention include but are not limited to a planar substrate, microbead, column or the like to which a first section of a synthetic RNA molecule, e.g., the 3′ or 5′ end, is tethered via direct or indirect linkage. Such substrates are disclosed herein or known in the art. The linkage is performed using methods known in the art, e.g., amino-carboxy coupling such as described in Wittebolle et al., Optimisation of the amino-carboxy coupling of oligonucleotides to beads used in liquid arrays, J Chem Tech Biotech 81:476-480 (2006); such techniques are readily known to a person having ordinary skill in the art.

Another portion of other the synthetic RNA molecule, e.g., the opposing 3′ or 5′ end, is attached directly or indirectly to a label or detectable molecule. The label is any molecule that is capable of being detected, and such labels or detectable molecules are known in the art and include without limitation: a fluorescent label, radiolabel or enzymatic label. Additional examples of such labels are disclosed herein above. In between the substrate-tethered portion and the labeled portion, the synthetic RNA molecule comprises a section or portion that is complementary to a target microRNA of interest. As desired, the complementary section can be perfectly complementary to the target microRNA, i.e., 100% complementary. The degree of association between the complementary section and the target microRNA can be manipulated, e.g., to allow the recognition of one specific target microRNA or to allow promiscuous recognition, e.g., of a family of target microRNAs. Means for such manipulation are disclosed herein or are known in the art, e.g., base pair mismatches, or assay conditions such as temperature or salt concentration. For example, the complementary section may carry mismatches with the target microRNA, e.g., such that the complementary section is at least 50%, 60%, 70%, 75%, 80%, 85%, 90%, 91%, 92%, 93%, 94%, 95%, 96%, 97%, 98% or at least 99% complementary to the target microRNA. The method comprises contacting the labeled and tethered synthetic RNA molecule with a sample comprising or suspected to comprise the target microRNA of interest. If the target microRNA is present in the sample and is also bound to an Ago protein, the Ago-microRNA can associate with the synthetic RNA molecule via base pairing between the target microRNA and the complementary region. Such association facilitates the cleavage of the synthetic RNA molecule via the endonucleolytic cleavage activity of the Ago protein. This cleavage liberates the label of the synthetic RNA molecule from the substrate. The amount of label associated with the substrate can be detected before and after contact with the sample comprising the target microRNA. Any such differences in the amount of label are indicative of the amount of Ago-bound target microRNA in the input sample.

Useful reaction conditions and buffers for the assay are known in the art. The reaction be performed at room temperature, 25° C., 30° C., 37° C. or up to 42° C.-45° C. for anywhere from 5 min to overnight depending on assay sensitivity and target abundance. For example, the reaction can be performed for 1-2 h at 37° C. See, e.g., Brown et al., Target accessibility dictates the potency of human RISC. Nature Structural & Molecular Biology 12, 469-470 (2005); Robb et al., Specific and potent RNAi in the nucleus of human cells. Nature Structural & Molecular Biology 12, 133-137 (2005); Lima et al., Binding and Cleavage Specificities of Human Argonaute2. J. Biol. Chem. 2009 284: 26017-26028.

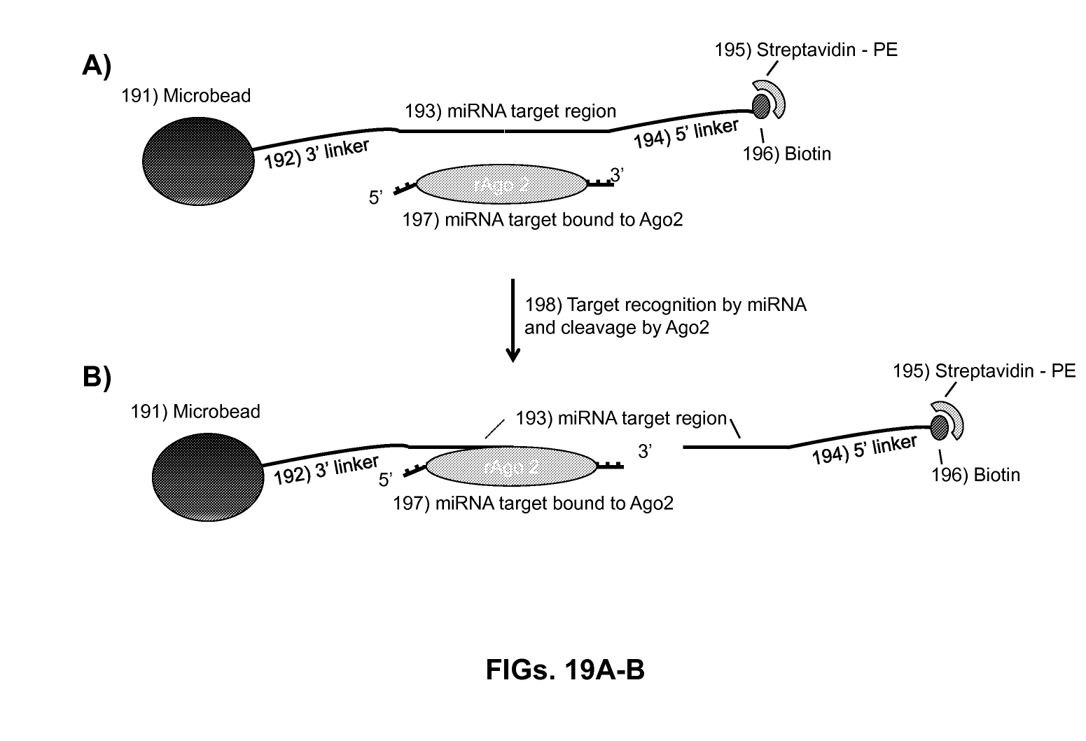

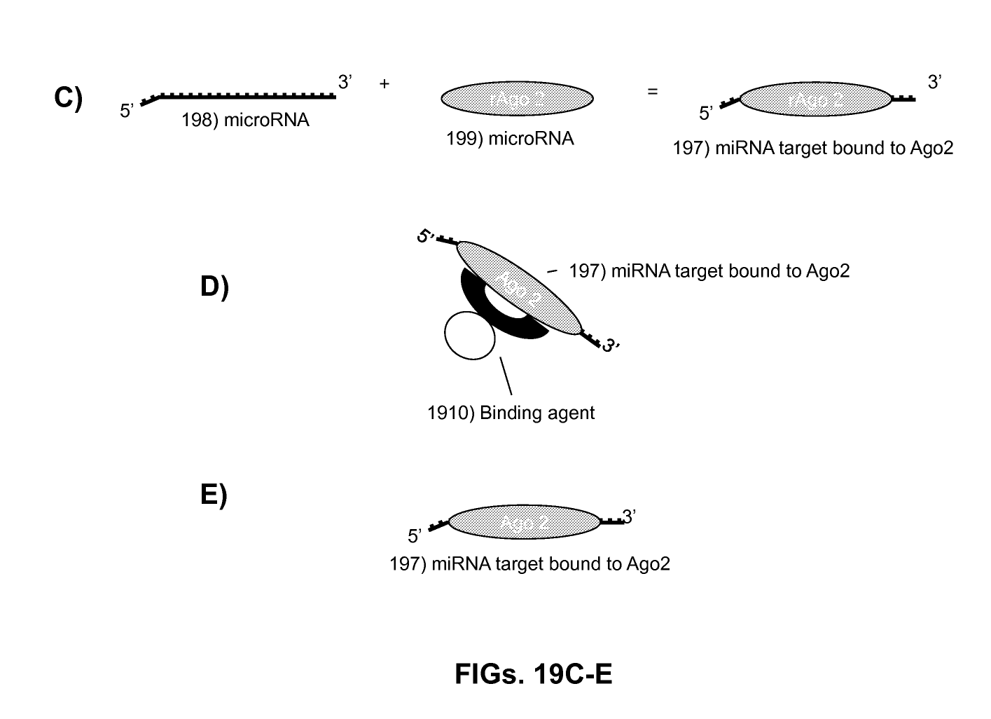

An exemplary embodiment of the assay is shown in FIG. 19. As shown in FIG. 19A, a synthetic RNA molecule contains a 3′ linker/extender region 192, a central miRNA targeting region 193 and a second 5′linker/extension region 194. The RNA is attached to a substrate, here microbead 191, on the 3′end 192 and the 5′end 194 is conjugated with biotin 196. The central miRNA targeting region 193 is designed to complement a miRNA sequence of interest. Region 193 can be complementary to any microRNA of interest. In the example shown in FIG. 19, streptavidin-PE (Phycoerythrin) 195 is used to label the biotin end of the synthetic RNA. As described, other labeling schemes can be employed. For example, the 5′end 194 can be directly labeled with Cy3, Cy5 or other detectable moiety disclosed herein or known in the art. As another example, the 5′end 194 can be indirectly labeled via base pairing with another complementary oligonucleotide that is labeled. If the target microRNA is present in the sample and is bound/associated with an Ago protein 197, e.g., any of Ago1-4 in the sample or added thereto, such as recombinant Ago2 (rAgo2), the target microRNA will bind the complementary microRNA targeting region 193 and subsequently cleave the synthetic RNA at region 193 through the endonucleolytic cleavage activity of Argonaute. See step 198 in FIG. 19. Once cleaved, the labeled end (here 5′) of the synthetic RNA molecule is released, thereby separating the biotin/Streptavidin-PE complex 195-196 from the microbead 191. See FIG. 19B. Next, the substrate microbeads can be isolated and washed to remove the cleaved and untethered end of the RNA, thereby leaving only the remaining uncleaved and still labeled material as well as any cleaved but now unlabeled RNA. After this wash step, the difference in PE signal correlates with the concentration and activity of the Ago-bound target microRNA 197 present in the original assay. The quantity of Ago-bound target microRNA in the input sample determines the level of RNA cleaved. For example, if the target microRNA is not present, or it is present but not bound in a functional form with Ago, the synthetic RNA target region 193 will remain uncleaved and the signal strength will be unchanged.

Any appropriate source of RNA and/or RNA pre-loaded into Argonaute can be tested using the assay. For example, the input sample may be cell lysate, bodily fluids, blood fractions (which may contain circulating Argonaute such as Ago 2 bound to miRNAs), plasma, serum, or isolated microvesicles. In some embodiments, Argonaute immunoprecipitated from a sample is used as an input source of RNP complexes for the assay. If the target microRNA is present and loaded into Argonaute in any of the aforementioned sources, the synthetic target 193 is cleaved and the label (e.g., biotin-strepavidin-PE 195-196 in the example of FIG. 19) is released.

FIGS. 19C-E illustrate schematically various sources of RNA that can be used as input for the assay. FIG. 19C illustrates microRNA 198 bound to an Ago protein 199 to form a ribonucleic acid complex 197. The Ago protein can be Ago1, Ago 2, Ago3 or Ago 4. FIG. 19D illustrates immunoprecipitation of an Argonaute—microRNA complex 197 using a binding agent to Ago 1910. The binding agent can be specific to a certain Argonaute, e.g., an antibody or aptamer to Ago2. In other embodiments, the binding agent recognizes more than one Ago family member, e.g., Ago1-4. In still other embodiments, the binding agent can bind indirectly to the one or more Ago protein. For example, the binding agent for the immunoprecipitation can be an antibody or aptamer to GW182 protein which forms a complex with Ago proteins. FIG. 19E illustrates direct analysis of Argonaute—microRNA complex 197, e.g., from a cell lysate, bodily fluid, or lysed microvesicle.

Alternately, the assay input can comprise RNA from a sample source bound that is then contacted with an Ago protein, such as purified Ago including recombinant Ago (rAgo). In this manner, RNA can be isolated from any appropriate source including without limitation cell lysate, bodily fluids, plasma, concentrated plasma, microvesicles, or HDL and LDL particles. Once isolated, the Ago protein, e.g., recombinant Argonaute 2, can be used to bind small RNA present in the sample. The Ago bound RNA can be used as input into the assay.

As described above, the third portion of the synthetic RNA molecule is labeled and thus cleavage of the complementary section allows removal of the label from the substrate. Thus, the amount of label removed from the substrate corresponds to the number of cleavage events. It will be appreciated that alternate methods of detecting the cleavage events are within the scope of the invention. In one embodiment, the label is added to the reaction mixture after the cleavage reaction has been allowed to occur. Following the example above, the streptavidin-PE 195 is added after the cleavage reaction has taken place. In another example, the third portion of the synthetic RNA molecule is not labeled. Rather, the cleavage events are observed by detecting the amount of cleaved synthetic RNA molecule remaining on the column after the cleavage reaction has occurred.

The degree of label liberated from the substrate can be detected and compared before and after the cleavage reaction has taken place. Alternately, the kinetics of the cleavage reaction can be observed using the subject methods. In an embodiment, the degree of label liberated from the substrate is detected in real time, thereby revealing the kinetics of the cleavage reaction.

Using the microRNA functional assay, virtually any microRNA can be screened with synthetic RNAs containing matched miRNA targeting regions. The assay can be performed in uniplex or multiplex fashion with multiple synthetic targets attached to distinguishable microbeads.

In an embodiment, the miR assay system is used for therapeutic RNAi molecule delivery and mode of action confirmation. Here, RNAi molecules are delivered systemically or in a targeted fashion to an appropriate cell type, tissue or other anatomical region. Target tissues can be analyzed for confirmation of delivery and confirmation of the RNAi therapeutic mode of action. For example, the presence of a therapeutic RNAi molecule at the tissue of interest can be detected by a phenotypic result directly driven by mRNA knockdown due to the activation of the RNAi therapeutic or alternatively through an unrelated apoptotic or inflammatory response of the cell. Lastly, IC50 of the activated therapeutic RNAi agent at the target tissue can be established using this methodology.

### Biosignatures for Cancer

As described herein, biosignatures comprising circulating biomarkers can be used to characterize a cancer. The biomarkers can be selected from those disclosed herein. For example, a non-exclusive list of biomarkers that can be used as part of a biosignature are listed in Tables 3, 4 and 5 herein. The biosignature can be used to characterize a cancer, e.g., for prostate, GI, or ovarian cancer. In some embodiments, the circulating biomarkers are associated with a vesicle or with a population of vesicles. For example, circulating biomarkers associated with vesicles can be used to capture and/or to detect a vesicle or a vesicle population.

It will be appreciated that the biomarkers presented herein, e.g., in Tables 3, 4 or 5, may be useful in biosignatures for other diseases, e.g., other proliferative disorders and cancers of other cellular or tissue origins. For example, transformation in various cell types can be due to common events, e.g., mutation in p53 or other tumor suppressor. A biosignature comprising cell-of-origin biomarkers and cancer biomarkers can be used to further assess the nature of the cancer. Biomarkers for metastatic cancer may be used with cell-of-origin biomarkers to assess a metastatic cancer. Such biomarkers for use with the invention include those in Dawood, Novel biomarkers of metastatic cancer, Exp Rev Mol Diag July 2010, Vol. 10, No. 5, Pages 581-590, which publication is incorporated herein by reference in its entirety.

For example, a biosignature comprising one or more of miR-378, miR-127-3p, miR-92a, and miR-486-3p can be used to characterize colorectal cancer. The presence of KRAS mutations can be associated with miR expression levels. See, e.g., Mosakhani et al., MicroRNA profiling differentiates colorectal cancer according to KRAS status. Genes Chromosomes Cancer. 2011 Sep. 15. doi: 10.1002/gcc.20925, which publication is incorporated herein by reference in its entirety. For example, KRAS mutations can be associated with upregulation miR-127-3p, miR-92a, and miR-486-3p and down-regulation of miR-378. Somatic KRAS mutations are found at high rates in various disorders, including without limitation leukemias, colon cancer, pancreatic cancer and lung cancer. KRAS mutations are predictive of poor response to panitumumab and cetuximab therapy. A KRAS+ phenotype is also associated with poor response to anti-EGFR therapies such as erlotinib and/or gefitinib. Thus, in an embodiment, levels of miRs correlated with KRAS status are used as part of a biosignature to provide a theranosis for cancers, e.g., metastatic colorectal cancer or lung cancer.

As another example, Pgrmc1 can be elevated in lung cancer tissue compared to normal tissue and in the plasma of lung cancer patients compared to non-cancer patients. See, e.g., Mir et al., Elevated Pgrmc1 (progesterone receptor membrane component 1)/sigma-2 receptor levels in lung tumors and plasma from lung cancer patients. Int J Cancer. 2011 Sep. 14. doi: 10.1002/ijc.26432, which publication is incorporated herein by reference in its entirety. In an embodiment, a presense or level of circulating Pgrmc1 is assessed in a patient sample in order to characterize a cancer. The cancer can be a lung cancer, including without limitation a squamous cell lung cancer (SCLC) or a lung adenocarcinoma. Elevated levels of Pgrmc1 compared to a control can indicate the presense of the cancer. The sample can be a tissue sample or a bodily fluid, e.g, sputum, peripheral blood, or a blood derivative. In an embodiment, the Pgrmc1 is associated with a population of vesicles.

The biosignatures of the invention may comprise markers that are upregulated, downregulated, or have no change, depending on the reference. Solely for illustration, if the reference is a normal sample, the biosignature may indicate that the subject is normal if the subject's biosignature is not changed compared to the reference. Alternately, the biosignature may comprise a mutated nucleic acid or amino acid sequence so that the levels of the components in the biosignature are the same between a normal reference and a diseased sample. In another case, the reference can be a cancer sample, such that the subject's biosignature indicates cancer if the subject's biosignature is substantially similar to the reference. The biosignature of the subject can comprise components that are both upregulated and downregulated compared to the reference. Solely for illustration, if the reference is a normal sample, a cancer biosignature can comprise both upregulated oncogenes and downregulated tumor suppressors. Vesicle markers can also be differentially expressed in various settings. For example, tetraspanins may be overexpressed in cancer vesicles compared to non-cancer vesicles, whereas MFG-E8 can be overexpressed in non-cancer vesicles as compared to cancer vesicles.

Prostate Cancer Biosignatures

In an aspect, the invention provides a method of detecting a microvesicle population in a biological sample. In an embodiment, the method comprises detecting a biosignature comprising a presence or level of multiple biomarkers. The biosignature can be used to characterize a cancer, e.g., a prostate cancer.

In an embodiment, the method comprises: (a) contacting a microvesicle population in a biological sample with a first binding agent and a second binding agent, (b) determining a presence or level of the microvesicle population bound by the first and second binding agents; and (c) identifying a biosignature comprising the presence or level of the bound microvesicle population. The first and second binding agents can comprise a pair of binding agents. The pair of binding agents can be used to identify a microvesicle population using various methods disclosed herein or known in the art. For example, the pair can be used to label a pair of antigens on a microvesicle surface. The labeled microvesicle population can be detected using flow cytometry or the like. Alternately, one member of the pair can be bound to a substrate (e.g., a capture agent) and the other member can be used to label the microvesicle, wherein the label allows detection of microvesicles bound by the pair of binding agents. The substrate can be a well, array, bead, column, paper, or the like as described herein or known in the art. The label can be a fluorescent, radiolabeled, enzymatic, or the like as described herein or known in the art. The label can also be indirect. For example, the labeled member of the binding pair may comprise a biotin molecule to allow its labeling with an avidin-bound label. Similarly, the labeled member of the binding pair can be detected by another labeled binding agent, e.g., a mouse IgG antibody binding agent can be labeled with a directly labeled anti-mouse IgG antibody. Any such configurations are contemplated by the invention.

In an embodiment, the first binding agent comprises a capture agent and the second binding agent comprises a detector agent. The capture and detector agents can be selected from one or more, e.g., at least 1, 2, 3, 4, 5, 6, 7, 8, 9, 10, 11, 12, 15, 20, 25, or all, pair of capture and detector agents in any of Tables 28-40 and 44-46. For example, the capture and detector agents can be selected from one or more, e.g., at least 1, 2, 3, 4, 5, 6, 7, 8, 9, 10, 11, 12, 15, 20, 25, or all, pair of capture and detector agents in Tables 44-46. Multiple pairs of capture and detector agents may improve the ability to characterize a phenotype. Thus, the invention contemplates use of any pairs of capture and detector agents that provide the desired diagnostic, prognostic or theranostic readout. As described herein, the use of the capture/detector pairs allows detection of microvesicle populations carrying more than one biomarker, e.g., one marker can be a tissue-specific or cell-of-origin marker, and the other marker can be a cancer marker. This scenario would allow detection of microvesicles that are shed from cancer cells from a given anatomical tissue or location. Thus, one of skill will appreciate that the targets of the capture/detector pairs can be switched while still detecting the same microvesicle population of interest. As a non-limiting example, the same population of microvesicles detected with KLK2 capture and EpCAM detector can be detected using EpCAM capture and KLK2 detector. Accordingly, the capture/detector pairs indicated in any of Tables 28-40 and 44-46 can be switched as desired.

As described, the biosignature can comprise one or more pair of binding agents as desired. In some embodiments, the one or more pair of binding agents comprises binding agents to one or more, e.g., 1, 2 or all, of Mammaglobin-MFG-E8, SIM2-MFG-E8 and NK-2R-MFG-E8. In another embodiment, the one or more pair of binding agents comprises binding agents to one or more, e.g., 1, 2 or all, of Integrin-MFG-E8, NK-2R-MFG-E8 and Gal3-MFG-E8. The one or more pair of capture and detector agents may comprise capture agents to one or more, e.g., 1, 2, 3, 4, or all, of AURKB, A33, CD63, Gro-alpha, and Integrin; and detector agents to one or more, e.g., 1, 2, or all, of MUC2, PCSA, and CD81. The one or more pair of capture and detector agents may also comprise capture agents to one or more, e.g., 1, 2, 3, 4, 5, 6, 7, 8 or all, of AURKB, CD63, FLNA, A33, Gro-alpha, Integrin, CD24, SSX2, and SIM2; and detector agents to one or more, e.g., 1, 2, 3, 4 or all, of MUC2, PCSA, CD81, MFG-E8, and EpCam. In some embodiments, the one or more pair of capture and detector agents comprises binding agents to one or more, e.g., 1, 2 or all, of EpCam-MMP7, PCSA-MMP7, and EpCam-BCNP. In some embodiment, the one or more pair of capture and detector agents comprises binding agents to one or more, e.g., 1, 2, 3, 4, or all, of EpCam-MMP7, PCSA-MMP7, EpCam-BCNP, PCSA-ADAM10, and PCSA-KLK2. In still other embodiments, the one or more pair of capture and detector agents comprises binding agents to one or more, e.g., 1, 2, 3, 4, 5, 6, 7, 8, 9 or all, of EpCam-MMP7, PCSA-MMP7, EpCam-BCNP, PCSA-ADAM10, PCSA-KLK2, PCSA-SPDEF, CD81-MMP7, PCSA-EpCam, MFGE8-MMP7 and PCSA-IL-8. The one or more pair of capture and detector agents may also comprise binding agents to one or more, e.g., 1, 2, 3, 4, or all, of EpCam-MMP7, PCSA-MMP7, EpCam-BCNP, PCSA-ADAM10, and CD81-MMP7.

The biosignature can comprise one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. The biosignature can include one or more of these biomarkers as a capture target and/or a detector target. In embodiments, a binding agent to one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8 is used to capture a population of vesicles. The captured vesicles can then detected with another binding agent to one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. Any combination of capture and detector is possible. In one embodiment, the biosignature comprises the following markers: 1) Epcam detector-MMP7 capture; 2) PCSA detector-MMP7 capture; 3) Epcam detector-BCNP capture. In another embodiment, the biosignature comprises the following markers: 1) Epcam detector-MMP7 capture; 2) PCSA detector-MMP7 capture; 3) Epcam detector-BCNP capture; 4) PCSA detector-Adam10 capture; and 5) PCSA detector-KLK2 capture. In still another embodiment, the biosignature comprises the following markers: 1) Epcam detector-MMP7 capture; 2) PCSA detector-MMP7 capture; 3) Epcam detector-BCNP capture; 4) PCSA detector-Adam10 capture; and 5) CD81 detector-MMP7 capture. EpCAM can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. MMP7 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. PCSA can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. BCNP can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. ADAM10 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. KLK2 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. SPDEF can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. CD81 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. MFGE8 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. IL-8 can be used as a detector target when the capture target is one or more, e.g., 1, 2, 3, 4, 5, 6, 7, 8, 9 or all, of EpCAM, MMP7, PCSA, BCNP, ADAM10, KLK2, SPDEF, CD81, MFGE8, and IL-8. The binding agents can comprise without limitation an antibody, aptamer, or combination thereof. In embodiments, the capture binding agent is tethered to a substrate and the detector binding agent is labeled.

The biosignature can comprise one or more, e.g., 1, 2, 3, 4, 5, 6, 7, 8, 9, 10, 11, 12, 13, 14 or all, of ADAM-10, BCNP, CD9, EGFR, EpCam, IL1B, KLK2, MMP7, p53, PBP, PCSA, SERPINB3, SPDEF, SSX2, and SSX4. The biosignature can include one or more of these biomarkers as a capture target and/or a detector target. In embodiments, a binding agent to one or more, e.g., 1, 2, 3, 4, 5, 6, 7, 8, 9, 10, 11, 12, 13, 14 or all, of ADAM-10, BCNP, CD9, EGFR, EpCam, IL1B, KLK2, MMP7, p53, PBP, PCSA, SERPINB3, SPDEF, SSX2, and SSX4 is used to capture a population of vesicles. The captured vesicles can then detected with another binding agent to one or more, e.g., 1, 2, 3, 4, 5, 6, 7, 8, 9, 10, 11, 12, 13, 14 or all, of ADAM-10, BCNP, CD9, EGFR, EpCam, IL1B, KLK2, MMP7, p53, PBP, PCSA, SERPINB3, SPDEF, SSX2, and SSX4. For example, the captured vesicles can be detected with a binding agent to EpCAM. The captured vesicles can be detected with a binding agent to PCSA. The captured vesicles can be detected with a binding agent to ADAM-10. The captured vesicles can be detected with a binding agent to BCNP. The captured vesicles can be detected with a binding agent to CD9. The captured vesicles can be detected with a binding agent to EGFR. The captured vesicles can be detected with a binding agent to IL1B. The captured vesicles can be detected with a binding agent to KLK2. The captured vesicles can be detected with a binding agent to MMP7. The captured vesicles can be detected with a binding agent to p53. The captured vesicles can be detected with a binding agent to PBP. The captured vesicles can be detected with a binding agent to SERPINB3. The captured vesicles can be detected with a binding agent to SPDEF. The captured vesicles can be detected with a binding agent to SSX2. The captured vesicles can be detected with a binding agent to SSX4. In some embodiments, the captured vesicles are detected with a binding agent to one or more of a general vesicle marker, e.g., as described in Table 3. The captured vesicles can also be detected with a binding agent to one or more, e.g., 1, 2, 3, 4, or 5, of EpCam, CD81, PCSA, MUC2, and MFG-E8. The captured vesicles can also be detected with a binding agent to one or more tetraspanin, e.g., 1, 2 or 3 of CD9, CD63, CD81, or other tetraspanin as described herein. In some embodiments, the vesicles are captured and detected with one or more pair of binding agents in Table 44. The one or more, e.g., 1, 2, 3, 4, 5, 6, 7, 8, 9, 10, 12, 15, 20 or more, pair of binding agents can be selected from the group consisting of EpCAM-EpCAM, EpCAM-KLK2, EpCAM-PBP, EpCAM-SPDEF, EpCAM-SSX2, EpCAM-SSX4, EpCAM-ADAM-10, EpCAM-SERPINB3, EpCAM-PCSA, EpCAM-p53, EpCAM-MMP7, EpCAM-IL1B, EpCAM-EGFR, EpCAM-CD9, EpCAM-BCNP, KLK2-EpCAM, KLK2-KLK2, KLK2-PBP, KLK2-SPDEF, KLK2-SSX2, KLK2-SSX4, KLK2-ADAM-10, KLK2-SERPINB3, KLK2-PCSA, KLK2-p53, KLK2-MMP7, KLK2-IL1B, KLK2-EGFR, KLK2-CD9, KLK2-BCNP, PBP-EpCAM, PBP-KLK2, PBP-PBP, PBP-SPDEF, PBP-SSX2, PBP-SSX4, PBP-ADAM-10, PBP-SERPINB3, PBP-PCSA, PBP-p53, PBP-MMP7, PBP-IL1B, PBP-EGFR, PBP-CD9, PBP-BCNP, SPDEF-EpCAM, SPDEF-KLK2, SPDEF-PBP, SPDEF-SPDEF, SPDEF-SSX2, SPDEF-SSX4, SPDEF-ADAM-10, SPDEF-SERPINB3, SPDEF-PCSA, SPDEF-p53, SPDEF-MMP7, SPDEF-IL1B, SPDEF-EGFR, SPDEF-CD9, SPDEF-BCNP, SSX2-EpCAM, SSX2-KLK2, SSX2-PBP, SSX2-SPDEF, SSX2-SSX2, SSX2-SSX4, SSX2-ADAM-10, SSX2-SERPINB3, SSX2-PCSA, SSX2-p53, SSX2-MMP7, SSX2-IL1B, SSX2-EGFR, SSX2-CD9, SSX2-BCNP, SSX4-EpCAM, SSX4-KLK2, SSX4-PBP, SSX4-SPDEF, SSX4-SSX2, SSX4-SSX4, SSX4-ADAM-10, SSX4-SERPINB3, SSX4-PCSA, SSX4-p53, SSX4-MMP7, SSX4-IL1B, SSX4-EGFR, SSX4-CD9, SSX4-BCNP, ADAM-10-EpCAM, ADAM-10-KLK2, ADAM-10-PBP, ADAM-10-SPDEF, ADAM-10 SSX2, ADAM-10-SSX4, ADAM-10-ADAM-10, ADAM-10-SERPINB3, ADAM-10-PCSA, ADAM-10-p53, ADAM-10-MMP7, ADAM-10-IL1B, ADAM-10-EGFR, ADAM-10-CD9, ADAM-10-BCNP, SERPINB3-EpCAM, SERPINB3-KLK2, SERPINB3-PBP, SERPINB3-SPDEF, SERPINB3-SSX2, SERPINB3-SSX4, SERPINB3-ADAM-10, SERPINB3-SERPINB3, SERPINB3-PCSA, SERPINB3-p53, SERPINB3-MMP7, SERPINB3-IL1B, SERPINB3-EGFR, SERPINB3-CD9, SERPINB3-BCNP, PCSA-EpCAM, PCSA-KLK2, PCSA-PBP, PCSA-SPDEF, PCSA-SSX2, PCSA-SSX4, PCSA-ADAM-10, PCSA-SERPINB3, PCSA-PCSA, PCSA-p53, PCSA-MMP7, PCSA-IL1B, PCSA-EGFR, PCSA-CD9, PCSA-BCNP, p53-EpCAM, p53-KLK2, p53-PBP, p53-SPDEF, p53-SSX2, p53-SSX4, p53-ADAM-10, p53-SERPINB3, p53-PCSA, p53-p53, p53-MMP7, p53-IL1B, p53-EGFR, p53-CD9, p53-BCNP, MMP7-EpCAM, MMP7-KLK2, MMP7-PBP, MMP7-SPDEF, MMP7-SSX2, MMP7-SSX4, MMP7-ADAM-10, MMP7-SERPINB3, MMP7-PCSA, MMP7-p53, MMP7-MMP7, MMP7-IL1B, MMP7-EGFR, MMP7-CD9, MMP7-BCNP, IL1B-EpCAM, IL1B-KLK2, IL1B-PBP, IL1B-SPDEF, IL1B-SSX2, IL1B-SSX4, IL1B-ADAM-10, IL1B-SERPINB3, IL1B-PCSA, IL1B-p53, IL1B-MMP7, IL1B-IL1B, IL1B-EGFR, IL1B-CD9, IL1B-BCNP, EGFR-EpCAM, EGFR-KLK2, EGFR-PBP, EGFR-SPDEF, EGFR-SSX2, EGFR-SSX4, EGFR-ADAM-10, EGFR-SERPINB3, EGFR-PCSA, EGFR-p53, EGFR-MMP7, EGFR-IL1B, EGFR-EGFR, EGFR-CD9, EGFR-BCNP, CD9-EpCAM, CD9-KLK2, CD9-PBP, CD9-SPDEF, CD9-SSX2, CD9-SSX4, CD9-ADAM-10, CD9-SERPINB3, CD9-PCSA, CD9-p53, CD9-MMP7, CD9-IL1B, CD9-EGFR, CD9-CD9, CD9-BCNP, BCNP-EpCAM, BCNP-KLK2, BCNP-PBP, BCNP-SPDEF, BCNP-SSX2, BCNP-SSX4, BCNP-ADAM-10, BCNP-SERPINB3, BCNP-PCSA, BCNP-p53, BCNP-MMP7, BCNP-IL1B, BCNP-EGFR, BCNP-CD9, BCNP-BCNP, and a combination thereof, wherein each pair is ordered as the target of the capture-detector agent. The binding agents can be an antibody, aptamer, a combination thereof, or other agent as disclosed herein or known in the art.

The biosignature can comprise a panel of capture and detector agents. In an embodiment, the panels comprise binding agents to more than one, e.g., 2, 3, 4, 5, 6, 7, 8, 9, 10, 11, 12, 13, 14 or all, of ADAM-10, BCNP, CD9, EGFR, EpCam, IL1B, KLK2, MMP7, p53, PBP, PCSA, SERPINB3, SPDEF, SSX2, and SSX4. For example, the biosignature may comprise a plurality of binding agents selected from the group consisting of SSX4-EpCAM, SSX4-KLK2, SSX4-PBP, SSX4-SPDEF, SSX4-SSX2, SSX4-EGFR, SSX4-MMP7, SSX4-BCNP1, SSX4-SERPINB3, KLK2-EpCAM, KLK2-PBP, KLK2-SPDEF, KLK2-SSX2, KLK2 EGFR, KLK2-MMP7, KLK2-BCNP1, KLK2-SERPINB3, PBP-EGFR, PBP-EpCAM, PBP-SPDEF, PBP-SSX2, PBP-SERPINB3, PBP-MMP7, PBP-BCNP1, EpCAM-SPDEF, EpCAM-SSX2, EpCAM SERPINB3, EpCAM-EGFR, EpCAM-MMP7, EpCAM-BCNP1, SPDEF-SSX2, SPDEF-SERPINB3, SPDEF-EGFR, SPDEF-MMP7, SPDEF-BCNP1, SSX2-EGFR, SSX2-MMP7, SSX2-BCNP1, SSX2-SERPINB3, SERPINB3-EGFR, SERPINB3-MMP7, SERPINB3-BCNP1, EGFR-MMP7, EGFR-BCNP1, MMP7-BCNP1, and a combination thereof. The binding agents can be used as capture agents. The captured vesicles can then detected with another binding agent to one or more, e.g., 1, 2, 3, 4, 5, 6, 7, 8, 9, 10, 11, 12, 13, 14 or all, of ADAM-10, BCNP, CD9, EGFR, EpCam, IL1B, KLK2, MMP7, p53, PBP, PCSA, SERPINB3, SPDEF, SSX2, and SSX4. For example, the captured vesicles can be detected with a binding agent to EpCAM. In some embodiments, the captured vesicles are detected with a binding agent to one or more of a general vesicle marker, e.g., as described in Table 3. The captured vesicles can also be detected with a binding agent to one or more, e.g., 1, 2, 3, 4, or 5, of EpCam, CD81, PCSA, MUC2, and MFG-E8. The captured vesicles can be detected with a binding agent to one or more, e.g., 1, 2, 3, 4, 5 or 6, of CD9, CD63, CD81, PCSA, MUC2, and MFG-E8. The captured vesicles can also be detected with a binding agent to one or more tetraspanin, e.g., 1, 2 or 3 of CD9, CD63, CD81, or other tetraspanin as described herein. In some embodiments, the vesicles are captured and detected with one or more pair of binding agents in Table 44. The binding agents can be an antibody, aptamer, a combination thereof, or other agent as disclosed herein or known in the art.

The biosignature can comprise one or more of EpCAM, KLK2, PBP, SPDEF, SSX2 and SSX4. The biosignature can include one or more of these biomarkers as a capture target and/or a detector target. In embodiments, a binding agent to one or more of EpCAM, KLK2, PBP, SPDEF, SSX2 and SSX4 is used to capture a population of vesicles. The captured vesicles can then detected with another binding agent to one or more of EpCAM, KLK2, PBP, SPDEF, SSX2 and SSX4. For example, captured vesicles can be detected with a binding agent to EpCAM. In an embodiment, the biosignature comprises a microvesicle population detected using a binding agent to EpCAM to capture the microvesicles and a binding agent to EpCAM to detect the microvesicles. In an embodiment, the biosignature comprises a microvesicle population detected using a binding agent to KLK2 to capture the microvesicles and a binding agent to EpCAM to detect the microvesicles. In an embodiment, the biosignature comprises a microvesicle population detected using a binding agent to PBP to capture the microvesicles and a binding agent to EpCAM to detect the microvesicles. In an embodiment, the biosignature comprises a microvesicle population detected using a binding agent to SPDEF to capture the microvesicles and a binding agent to EpCAM to detect the microvesicles. In an embodiment, the biosignature comprises a microvesicle population detected using a binding agent to SSX2 to capture the microvesicles and a binding agent to EpCAM to detect the microvesicles. In an embodiment, the biosignature comprises a microvesicle population detected using a binding agent to SSX4 to capture the microvesicles and a binding agent to EpCAM to detect the microvesicles. Any useful combination of these capture/detector pairs can be used as desired. In an embodiment, the combination of capture/detector pairs comprises: 1) EpCAM capture-EpCAM detector; and 2) KLK2, PBP, SPDEF, SSX2 or SSX4 capture-EpCAM detector. In an embodiment, the combination of capture/detector pairs comprises: 1) KLK2 capture-EpCAM detector; and 2) EpCAM, PBP, SPDEF, SSX2 or SSX4 capture-EpCAM detector. In an embodiment, the combination of capture/detector pairs comprises: 1) PBP capture-EpCAM detector; and 2) EpCAM, KLK2, SPDEF, SSX2 or SSX4 capture-EpCAM detector. In an embodiment, the combination of capture/detector pairs comprises: 1) SPDEF capture-EpCAM detector; and 2) EpCAM, KLK2, PBP, SSX2 or SSX4 capture-EpCAM detector. In an embodiment, the combination of capture/detector pairs comprises: 1) SSX2 capture-EpCAM detector; and 2) EpCAM, KLK2, PBP, SPDEF or SSX4 capture-EpCAM detector. In an embodiment, the combination of capture/detector pairs comprises: 1) SSX4 capture-EpCAM detector; and 2) EpCAM, KLK2, PBP, SPDEF or SSX2 capture-EpCAM detector. The binding agents can comprise without limitation an antibody, aptamer, or combination thereof. For example, the capture agents can comprise antibodies and the detector agent can comprise an aptamer. In embodiments, the capture binding agent is tethered to a substrate and the detector binding agent is labeled. If desired, the vesicles can be detected with a binding agent to PCSA.

In an embodiment, the microvesicles are detecting using capture and detector pairs specific for vesicles from a desired cell of origin. In an embodiment, the vesicles are captured using a cancer marker and detected with a tissue specific marker. Similarly, the vesicles can be captured using a tissue specific marker and detected with a cancer marker. For example, the cancer marker can be EpCAM or B7H3, and the tissue specific marker can be a prostate marker including without limitation PBP, PCSA, PSCA, PSMA, KLK2, PSA, or the like. Without being bound by theory, such embodiments allow for vesicles derived from prostate cancer cells to be detected in circulation.

Multiple detector agents can be used if desired. For example, the use of multiple general vesicle markers may amplify the detection signal. For example, detection with CD9, CD63 and CD81 together may provide more signal than detection via a single tetraspanin, which may be desirable in some applications.

In an embodiment, EpCAM (epithelial cellular adhesion molecule) is the target of the anti Epithelial cellular adhesion molecule antibody MAB 9601 in Table 27. Further information about EpCAM can be found at www.genecards.org/cgi-bin/carddisp.pl?gene=EPCAM.

In an embodiment, MMP7 (matrix metallopeptidase 7 (matrilysin, uterine); matrix metalloproteinase 7) is the target of the Anti Matrix metallo Proteinase 7 antibody NB300-1000 in Table 27. Further information about MMP7 can be found at www.genecards.org/cgi-bin/carddisp.pl?gene=MMP7. Commercially available antibodies to MMP7 that can be used to carry out the methods of the invention include: 1) Anti Matrix metallo Proteinase 7 antibody, R&D Systems, clone 111433, catalog number MAB9071; 2) Anti Matrix metallo Proteinase 7 antibody, R&D Systems, clone 111439, catalog number MAB9072; 3) Anti Matrix metallo Proteinase 7 antibody, R&D Systems, clone 6A4, catalog number MAB907; 4) Anti Matrix metallo Proteinase 7 antibody, Millipore, clone 141-7B2, catalog number MAB3315; 5) Anti Matrix metallo Proteinase 7 antibody, Millipore, clone 176-5F12, MAB3322; 6) Anti Matrix metallo Proteinase 7 polyclonal antibody, Novus, catalog number NB300-1000.

In an embodiment, PCSA (prostate cell surface antigen) is the target of the Anti prostate cell surface antibody. See Table 27. PCSA is also recognized by the 5E10 antibody described in Rokhlin, O W, et al. Cancer Lett., 131:129-36 (1998), which publication is incorporated by reference herein in its entirety.

In an embodiment, BCNP (B-cell novel protein 1; FAM129C; family with sequence similarity 129, member C; niban-like protein 2) is the target of the Anti B-cell novel protein1 antibody ab59781 in Table 27. BCNP has several splice forms and isoforms, e.g., BCNP1, BCNP2, BCNP3, BCNP4 and BCNP5. The protein isoforms can also be refered to as Q86XR2-1, Q86XR2-2, Q86XR2-3, Q86XR2-4 and Q86XR2-5. The antibody recognizes at least BCNP1, BCNP2, BCNP3, and may recognize the isoforms 4 and 5. Further information about BCNP is available at www.genecards.org/cgi-bin/carddisp.pl?gene=FAM129C.

In an embodiment, ADAM10 (ADAM metallopeptidase domain 10; a disintegrin and metalloproteinase domain 10) is the target of the Anti disintegrin and metalloproteinase domain 10 antibody MAB1427 in Table 27. Further information about ADAM10 can be found at www.genecards.org/cgi-bin/carddisp.pl?gene=ADAM10.

In an embodiment, KLK2 (kallikrein-related peptidase 2) is the target of the Anti kallikrein-related peptidase 2 antibody H00003817-M03 in Table 27. Further information about KLK2 can be found at www.genecards.org/cgi-bin/carddisp.pl?gene=KLK2.

In an embodiment, SPDEF (SAM pointed domain containing ets transcription factor) is the target of the Anti SAM pointed domain containing ets transcription factor antibody H00025803-M01 in Table 27. Further information about SPDEF can be found at www.genecards.org/cgi-bin/carddisp.pl?gene=SPDEF.

In an embodiment, CD81 (CD81 molecule; CD81 antigen; tetraspanin-28) is the target of the Anti cluster of differentiation 81 antibody 555675 in Table 27. Further information about CD81 can be found at www.genecards.org/cgi-bin/carddisp.pl?gene=CD81.

In an embodiment, MFGE8 (milk fat globule-EGF factor 8 protein; MFG-E8; sperm associated antigen 10; lactahedrin) is the target of the Anti Milk fat globule-EGF factor 8 protein antibody MAB27671 in Table 27. Further information about MFGE8 can be found at www.genecards.org/cgi-bin/carddisp.pl?gene=MFGE8.

In an embodiment, IL-8 (interleukin 8) is the target of the Anti Interleukin 8 antibody OMA1-03346 in Table 27. Further information about IL-8 can be found at www.genecards.org/cgi-bin/carddisp.pl?gene=IL8.

In an embodiment, SSX4 (synovial sarcoma, X breakpoint 4) is the target of the Anti synovial sarcoma, X breakpoint 4 antibody H00006759-MO2 in Table 27. Further information about SSX4 can be found at www.genecards.org/cgi-bin/carddisp.pl?gene=SSX4.

In an embodiment, SSX2 (synovial sarcoma, X breakpoint 2) is the target of the Anti synovial sarcoma X break point 2 antibody H00006757-MO1 in Table 27. Further information about SSX2 can be found at www.genecards.org/cgi-bin/carddisp.pl?gene=SSX2.

In an embodiment, EGFR (epidermal growth factor receptor) is the target of the Anti epidermal growth factor antibody 555996 in Table 27. Further information about EGFR can be found at www.genecards.org/cgi-bin/carddisp.pl?gene=EGFR.

In an embodiment, SERPINB3 (serpin peptidase inhibitor, Glade B (ovalbumin), member 3) is the target of the Anti serpin peptidase inhibitor, Glade B member 3 antibody WH0006317M1 in Table 27. Further information about SERPINB3 can be found at www.genecards.org/cgi-bin/carddisp.pl?gene=SERPINB3.

In an embodiment, IL1B (interleukin 1, beta) is the target of the Anti Interleukin-1B antibody WH0003553M1 in Table 27. Further information about IL1B can be found at www.genecards.org/cgi-bin/carddisp.pl?gene=IL1B.

In an embodiment, TP53 (p53; tumor protein p53) is the target of the Anti tumor protein 53 antibody 654802 in Table 27. Further information about TP53 can be found at www.genecards.org/cgi-bin/carddisp.pl?gene=TP53.

In an embodiment, PBP (prostatic binding protein; PEBP1; phosphatidylethanolamine binding protein 1) is the target of the Anti Prostatic binding protein antibody H00005037-M01 in Table 27. Further information about PBP can be found at www.genecards.org/cgi-bin/carddisp.pl?gene=PEBP1.

In an embodiment, CD9 (CD9 molecule) is the target of the Anti-cluster of differentiation 9 antibody MAB633 in Table 27. Further information about CD9 can be found at www.genecards.org/cgi-bin/carddisp.pl?gene=CD9.

Alternate antibodies, aptamers and other binding agents that recognize the above biomarkers are known in the art. See, e.g., the Genecard references above.

### Theranosis

As disclosed herein, methods are disclosed for characterizing a phenotype for a subject by assessing one or more biomarkers, including vesicle biomarkers and/or circulating biomarkers. The biomarkers can be assessed using methods for multiplexed analysis of vesicle biomarkers disclosed herein. Characterizing a phenotype can include providing a theranosis for a subject, such as determining if a subject is predicted to respond to a treatment or is predicted to be non-responsive to a treatment. A subject that responds to a treatment can be termed a responder whereas a subject that does not respond can be termed a non-responder. A subject suffering from a condition can be considered to be a responder for a treatment based on, but not limited to, an improvement of one or more symptoms of the condition; a decrease in one or more side effects of an existing treatment; an increased improvement, or rate of improvement, in one or more symptoms as compared to a previous or other treatment; or prolonged survival as compared to without treatment or a previous or other treatment. For example, a subject suffering from a condition can be considered to be a responder to a treatment based on the beneficial or desired clinical results including, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment.

The systems and methods disclosed herein can be used to select a candidate treatment for a subject in need thereof. Selection of a therapy can be based on one or more characteristics of a vesicle, such as the biosignature of a vesicle, the amount of vesicles, or both. Vesicle typing or profiling, such as the identification of the biosignature of a vesicle, the amount of vesicles, or both, can be used to identify one or more candidate therapeutic agents for an individual suffering from a condition. For example, vesicle profiling can be used to determine if a subject is a non-responder or responder to a particular therapeutic, such as a cancer therapeutic if the subject is suffering from a cancer.

Vesicle profiling can be used to provide a diagnosis or prognosis for a subject, and a therapy can be selected based on the diagnosis or prognosis. Alternatively, therapy selection can be directly based on a subject's vesicle profile. Furthermore, a subject's vesicle profile can be used to follow the evolution of a disease, to evaluate the efficacy of a medication, adapt an existing treatment for a subject suffering from a disease or condition, or select a new treatment for a subject suffering from a disease or condition.

A subject's response to a treatment can be assessed using biomarkers, including vesicles, microRNA, and other circulating biomarkers. In one embodiment, a subject is determined, classified, or identified as a non-responder or responder based on the subject's vesicle profile assessed prior to any treatment. During pretreatment, a subject can be classifed as a non-responder or responder, thereby reducing unnecessary treatment options, and avoidance of possible side effects from ineffective therapeutics. Furthermore, the subject can be identified as a responder to a particular treatment, and thus vesicle profiling can be used to prolong survival of a subject, improve the subject's symptoms or condition, or both, by providing personalized treatment options. Thus, a subject suffering from a condition can have a biosignature generated from vesicles and other circulating biomarkers using one or more systems and methods disclosed herein, and the profile can then be used to determine whether a subject is a likely non-responder or responder to a particular treatment for the condition. Based on use of the biosignature to predict whether the subject is a non-responder or responder to the initially contemplated treatment, a particular treatment contemplated for treating the subject's condition can be selected for the subject, or another potentially more optimal treatment can be selected.

In one embodiment, a subject suffering from a condition is currently being treated with a therapeutic. A sample can be obtained from the subject before treatment and at one or more timepoints during treatment. A biosignature including vesicles or other biomarkers from the samples can be assessed and used to determine the subject's response to the drug, such as based on a change in the biosignature over time. If the subject is not responding to the treatment, e.g., the biosignature does not indicate that the patient is responding, the subject can be classified as being non-responsive to the treatment, or a non-responder. Similarly, one or more biomarkers associated with a worsening condition may be detected such that the biosignature is indicative of patient's failure to respond favorably to the treatment. In another example, one or more biomarkers associated with the condition remain the same despite treatment, indicating that the condition is not improving. Thus, based on the biosignature, a treatment regimen for the subject can be changed or adapted, including selection of a different therapeutic.

Alternatively, the subject can be determined to be responding to the treatment, and the subject can be classified as being responsive to the treatment, or a responder. For example, one or more biomarkers associated with an improvement in the condition or disorder may be detected. In another example, one or more biomarkers associated with the condition changes, thus indicating an improvement. Thus, the existing treatment can be continued. In another embodiment, even when there is an indiciation of improvement, the existing treatment may be adapted or changed if the biosignature indicates that another line of treatment may be more effective. The existing treatment may be combined with another therapeutic, the dosage of the current therapeutic may be increased, or a different candidate treatment or therapeutic may be selected. Criteria for selecting the different candidate treatment can depend on the setting. In one embodiment, the candidate treatment may have been known to be effective for subjects with success on the existing treatment. In another embodiment, the candidate treatment may have been known to be effective for other subjects with a similar biosignature.

In some embodiments, the subject is undergoing a second, third or more line of treatment, such as cancer treatment. A biosignature according to the invention can be determined for the subject prior to a second, third or more line of treatment, to determine whether a subject would be a responder or non-resonder to the second, third or more line of treatment. In another embodiment, a biosignature is determined for the subject during the second, third or more line of treatment, to determine if the subject is responding to the second, third or more line of treatment.

The methods and systems described herein for assessing one or more vesicles can be used to determine if a subject suffering from a condition is responsive to a treatment, and thus can be used to select a treatment that improves one or more symptoms of the condition; decreases one or more side effects of an existing treatment; increases the improvement, or rate of improvement, in one or more symptoms as compared to a previous or other treatment; or prolongs survival as compared to without treatment or a previous or other treatment. Thus, the methods described herein can be used to prolong survival of a subject by providing personalized treatment options, and/or may reduce unnecessary treatment options and unnecessary side effects for a subject.

The prolonged survival can be an increased progression-free survival (PFS), which denotes the chances of staying free of disease progression for an individual or a group of individuals suffering from a disease, e.g., a cancer, after initiating a course of treatment. It can refer to the percentage of individuals in the group whose disease is likely to remain stable (e.g., not show signs of progression) after a specified duration of time. Progression-free survival rates are an indication of the effectiveness of a particular treatment. In other embodiments, the prolonged survival is disease-free survival (DFS), which denotes the chances of staying free of disease after initiating a particular treatment for an individual or a group of individuals suffering from a cancer. It can refer to the percentage of individuals in the group who are likely to be free of disease after a specified duration of time. Disease-free survival rates are an indication of the effectiveness of a particular treatment. Two treatment strategies can be compared on the basis of the disease-free survival that is achieved in similar groups of patients. Disease-free survival is often used with the term overall survival when cancer survival is described.

The candidate treatment selected by vesicle profiling as described herein can be compared to a non-vesicle profiling selected treatment by comparing the progression free survival (PFS) using therapy selected by vesicle profiling (period B) with PFS for the most recent therapy on which the subject has just progressed (period A). In one setting, a PFSB/PFSA ratio≧1.3 is used to indicate that the vesicle profiling selected therapy provides benefit for subject (see for example, Robert Temple, Clinical measurement in drug evaluation. Edited by Wu Ningano and G. T. Thicker John Wiley and Sons Ltd. 1995; Von Hoff D. D. Clin Can Res. 4: 1079, 1999: Dhani et al. Clin Cancer Res. 15: 118-123, 2009).

Other methods of comparing the treatment selected by vesicle profiling can be compared to a non-vesicle profiling selected treatment by determine response rate (RECIST) and percent of subjects without progression or death at 4 months. The term “about” as used in the context of a numerical value for PFS means a variation of +/−ten percent (10%) relative to the numerical value. The PFS from a treatment selected by vesicle profiling can be extended by at least 10%, 15%, 20%, 30%, 40%, 50%, 60%, 70%, 80%, or at least 90% as compared to a non-vesicle profiling selected treatment. In some embodiments, the PFS from a treatment selected by vesicle profiling can be extended by at least 100%, 150%, 200%, 300%, 400%, 500%, 600%, 700%, 800%, 900%, or at least about 1000% as compared to a non-vesicle profiling selected treatment. In yet other embodiments, the PFS ratio (PFS on vesicle profiling selected therapy or new treatment/PFS on prior therapy or treatment) is at least about 1.3. In yet other embodiments, the PFS ratio is at least about 1.1, 1.2, 1.3, 1.4, 1.5, 1.6, 1.7, 1.8, 1.9, or 2.0. In yet other embodiments, the PFS ratio is at least about 3, 4, 5, 6, 7, 8, 9 or 10.

Similarly, the DFS can be compared in subjects whose treatment is selected with or without determining a biosignature according to the invention. The DFS from a treatment selected by vesicle profiling can be extended by at least 10%, 15%, 20%, 30%, 40%, 50%, 60%, 70%, 80%, or at least 90% as compared to a non-vesicle profiling selected treatment. In some embodiments, the DFS from a treatment selected by vesicle profiling can be extended by at least 100%, 150%, 200%, 300%, 400%, 500%, 600%, 700%, 800%, 900%, or at least about 1000% as compared to a non-vesicle profiling selected treatment. In yet other embodiments, the DFS ratio (DFS on vesicle profiling selected therapy or new treatment/DFS on prior therapy or treatment) is at least about 1.3. In yet other embodiments, the DFS ratio is at least about 1.1, 1.2, 1.3, 1.4, 1.5, 1.6, 1.7, 1.8, 1.9, or 2.0. In yet other embodiments, the DFS ratio is at least about 3, 4, 5, 6, 7, 8, 9 or 10.

In some embodiments, the candidate treatment selected by microvescile profiling does not increase the PFS ratio or the DFS ratio in the subject; nevertheless vesicle profiling provides subject benefit. For example, in some embodiments no known treatment is available for the subject. In such cases, vesicle profiling provides a method to identify a candidate treatment where none is currently identified. The vesicle profiling may extend PFS, DFS or lifespan by at least 1 week, 2 weeks, 3 weeks, 4 weeks, 1 month, 5 weeks, 6 weeks, 7 weeks, 8 weeks, 2 months, 9 weeks, 10 weeks, 11 weeks, 12 weeks, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or 2 years. The vesicle profiling may extend PFS, DFS or lifespan by at least 2½ years, 3 years, 4 years, 5 years, or more. In some embodiments, the methods of the invention improve outcome so that subject is in remission.

The effectiveness of a treatment can be monitored by other measures. A complete response (CR) comprises a complete disappearance of the disease: no disease is evident on examination, scans or other tests. A partial response (PR) refers to some disease remaining in the body, but there has been a decrease in size or number of the lesions by 30% or more. Stable disease (SD) refers to a disease that has remained relatively unchanged in size and number of lesions. Generally, less than a 50% decrease or a slight increase in size would be described as stable disease. Progressive disease (PD) means that the disease has increased in size or number on treatment. In some embodiments, vesicle profiling according to the invention results in a complete response or partial response. In some embodiments, the methods of the invention result in stable disease. In some embodiments, the invention is able to achieve stable disease where non-vesicle profiling results in progressive disease.

The theranosis based on a biosignature of the invention can be for a phenotype including without limitation those listed herein. Characterizing a phenotype includes determining a theranosis for a subject, such as predicting whether a subject is likely to respond to a treatment (“responder”) or be non-responsive to a treatment (“non-responder”). As used herein, identifying a subject as a “responder” to a treatment or as a “non-responder” to the treatment comprises identifying the subject as either likely to respond to the treatment or likely to not respond to the treatment, respectively, and does not require determining a definitive prediction of the subject's response. One or more vesicles, or populations of vesicles, obtained from subject are used to determine if a subject is a non-responder or responder to a particular therapeutic, by assessing biomarkers disclosed herein, e.g., those listed in Table 7. Detection of a high or low expression level of a biomarker, or a mutation of a biomarker, can be used to select a candidate treatment, such as a pharmaceutical intervention, for a subject with a condtion. Table 7 contains illustrative conditions and pharmaceutical interventions for those conditions. The table lists biomarkers that affect the efficacy of the intervention. The biomarkers can be assessed using the methods of the invention, e.g., as circulating biomarkers or in association with a vesicle.

TABLE 7
Examples of Biomarkers and Pharmaceutical Intervention for a Condition

| Condition | Pharmaceutial intervention | Biomarker |
|---|---|---|
| Peripheral Arterial | Atorvastatin, Simvastatin, Rosuvastatin, | C-reactive protein(CRP), serum |
| Disease | Pravastatin, Fluvastatin, Lovastatin | Amylyoid A (SAA), interleukin-6, |
| | | intracellular adhesion molecule |
| | | (ICAM), vascular adhesion |
| | | molecule (VCAM), CD40L, |
| | | fibrinogen, fibrin D-dimer, |
| | | fibrinopeptide A, von Willibrand |
| | | factor, tissue plasminogen activator |
| | | antigen (t-PA), factor VII, |
| | | prothrombin fragment 1, oxidized |
| | | low density lipoprotein (oxLDL), |
| | | lipoprotein A |
| Non-Small Cell | Erlotinib, Carboplatin, Paclitaxel, Gefitinib | EGFR, excision repair cross- |
| Lung Cancer | | complementation group 1 |
| | | (ERCC1), p53, Ras, p27, class III |
| | | beta tubulin, breast cancer gene 1 |
| | | (BRCA1), breast cancer gene 1 |
| | | (BRCA2), ribonucleotide reductase |
| | | messenger 1 (RRM1) |
| Colorectal Cancer | Panitumumab, Cetuximab | K-ras |
| Breast Cancer | Trastuzumab, Anthracyclines, Taxane, | HER2, toposiomerase II alpha, |
| | Methotrexate, fluorouracil | estrogen receptor, progesterone |
| | | receptor |
| Alzheimer's Disease | Donepezil, Galantamine, Memantine, | beta-amyloid protein, amyloid |
| | Rivastigmine, Tacrine | precursor protein (APP), |
| | | APP670/671, APP693, APP692, |
| | | APP715, APP716, APP717, |
| | | APP723, presenilin 1, presenilin 2, |
| | | cerebrospinal fluid amyloid beta |
| | | protein 42 (CSF-Abeta42), |
| | | cerebrospinal fluid amyloid beta |
| | | protein 40 (CSF-Abeta40), F2 |
| | | isoprostane, 4-hydroxynonenal, F4 |
| | | neuroprostane, acrolein |
| Arrhythmia | Disopyramide, Flecainide, Lidocaine, Mexiletine, | SERCA, AAP, Connexin 40, |
| | Moricizine, Procainamide, Propafenone, | Connexin 43, ATP-sensitive |
| | Quinidine, Tocainide, Acebutolol, Atenolol, | potassium channel, Kv1.5 channel, |
| | Betaxolol, Bisoprolol, Carvedilol, Esmolol, | acetylcholine-activated posassium |
| | Metoprolol, Nadolol, Propranolol, Sotalol, | channel |
| | Timolol, Amiodarone, Azimilide, Bepridil, | |
| | Dofetilide, Ibutilide, Tedisamil, Diltiazem, | |
| | Verapamil, Azimilide, Dronedarone, | |
| | Amiodarone, PM101, ATI-2042, Tedisamil, | |
| | Nifekalant, Ambasilide, Ersentilide, Trecetilide, | |
| | Almokalant, D-sotalol, BRL-32872, HMR1556, | |
| | L768673, Vernakalant, AZD70009, AVE0118, | |
| | S9947, NIP-141/142, XEN-D0101/2, Ranolazine, | |
| | Pilsicainide, JTV519, Rotigaptide, GAP-134 | |
| Rheumatoid arthritis | Methotrexate, infliximab, adalimumab, | 677CC/1298AA MTHFR, |
| | etanercept, sulfasalazine | 677CT/1298AC MTHFR, 677CT |
| | | MTHFR, G80AA RFC-1, 3435TT |
| | | MDR1 (ABCB1), 3435TT ABCB1, |
| | | AMPD1/ATIC/ITPA, IL1-RN3, |
| | | HLA-DRB103, CRP, HLA-D4, |
| | | HLA DRB-1, anti-citrulline epitope |
| | | containing peptides, anti-A1/RA33, |
| | | Erythrocyte sedimentation rate |
| | | (ESR), C-reactive protein (CRP), |
| | | SAA (serum amyloid-associated |
| | | protein), rheumatoid factor, IL-1, |
| | | TNF, IL-6, IL-8, IL-1Ra, |
| | | Hyaluronic acid, Aggrecan, Glc- |
| | | Gal-PYD, osteoprotegerin, |
| | | RNAKL, carilage oligomeric |
| | | matrix protein (COMP), |
| | | calprotectin |
| Arterial Fibrillation | warfarin, aspirin, anticoagulants, heparin, | F1.2, TAT, FPA, beta- |
| | ximelagatran | throboglobulin, platelet factor 4, |
| | | soluble P-selectin, IL-6, CRP |
| HIV Infection | Zidovudine, Didanosine, Zalcitabine, Stavudine, | HIV p24 antigen, TNF-alpha, |
| | Lamivudine, Saquinavir, Ritonavir, Indinavir, | TNFR-II, CD3, CD14, CD25, |
| | Nevirane, Nelfinavir, Delavirdine, Stavudine, | CD27, Fas, FasL, beta2 |
| | Efavirenz, Etravirine, Enfuvirtide, Darunavir, | microglobulin, neopterin, HIV |
| | Abacavir, Amprenavir, Lonavir/Ritonavirc, | RNA, HLA-B *5701 |
| | Tenofovir, Tipranavir | |
| Cardiovascular | lisinopril, candesartan, enalapril | ACE inhibitor, angiotensin |
| Disease | | |

Cancer

Vesicle biosignatures can be used in the theranosis of a cancer, such as identifying whether a subject suffering from cancer is a likely responder or non-responder to a particular cancer treatment. The subject methods can be used to theranose cancers including those listed herein, e.g., in the “Phenotype” section above. These include without limitation lung cancer, non-small cell lung cancerm small cell lung cancer (including small cell carcinoma (oat cell cancer), mixed small cell/large cell carcinoma, and combined small cell carcinoma), colon cancer, breast cancer, prostate cancer, liver cancer, pancreatic cancer, brain cancer, kidney cancer, ovarian cancer, stomach cancer, melanoma, bone cancer, gastric cancer, breast cancer, glioma, glioblastoma, hepatocellular carcinoma, papillary renal carcinoma, head and neck squamous cell carcinoma, leukemia, lymphoma, myeloma, or other solid tumors.

A biosignature of circulating biomarkers, including markers associated with vesicle, in a sample from a subject suffering from a cancer can be used select a candidate treatment for the subject. The biosignature can be determined according to the methods of the invention presented herein. In some embodiments, the candidate treatment comprises a standard of care for the cancer. The biosignature can be used to determine if a subject is a non-responder or responder to a particular treatment or standard of care. The treatment can be a cancer treatment such as radiation, surgery, chemotherapy or a combination thereof. The cancer treatment can be a therapeutic such as anti-cancer agents and chemotherapeutic regimens. Cancer treatments for use with the methods of the invention include without limitation those listed in Table 8:

TABLE 8
Cancer Treatments

| | Treatment or Agent |
|---|---|
| Cancer therapies | Radiation, Surgery, Chemotherapy, Biologic therapy, Neo-adjuvant therapy, Adjuvant |
| | therapy, Palliative therapy, Watchful waiting |
| Anti-cancer agents | 13-cis-Retinoic Acid, 2-CdA, 2-Chlorodeoxyadenosine, 5-Azacitidine, 5-Fluorouracil, |
| (chemotherapies and | 5-FU, 6-Mercaptopurine, 6-MP, 6-TG, 6-Thioguanine, Abraxane, Accutane ®, |
| biologics) | Actinomycin-D, Adriamycin ®, Adrucil ®, Afinitor ®, Agrylin ®, Ala-Cort ®, |
| | Aldesleukin, Alemtuzumab, ALIMTA, Alitretinoin, Alkaban-AQ ®, Alkeran ®, All- |
| | transretinoic Acid, Alpha Interferon, Altretamine, Amethopterin, Amifostine, |
| | Aminoglutethimide, Anagrelide, Anandron ®, Anastrozole, Arabinosylcytosine, Ara-C, |
| | Aranesp ®, Aredia ®, Arimidex ®, Aromasin ®, Arranon ®, Arsenic Trioxide, |
| | Asparaginase, ATRA, Avastin ®, Azacitidine, BCG, BCNU, Bendamustine, |
| | Bevacizumab, Bexarotene, BEXXAR ®, Bicalutamide, BiCNU, Blenoxane ®, |
| | Bleomycin, Bortezomib, Busulfan, Busulfex ®, C225, Calcium Leucovorin, Campath ®, |
| | Camptosar ®, Camptothecin-11, Capecitabine, Carac ™, Carboplatin, Carmustine, |
| | Carmustine Wafer, Casodex ®, CC-5013, CCI-779, CCNU, CDDP, CeeNU, |
| | Cerubidine ®, Cetuximab, Chlorambucil, Cisplatin, Citrovorum Factor, Cladribine, |
| | Cortisone, Cosmegen ®, CPT-11, Cyclophosphamide, Cytadren ®, Cytarabine, |
| | Cytarabine Liposomal, Cytosar-U ®, Cytoxan ®, Dacarbazine, Dacogen, Dactinomycin, |
| | Darbepoetin Alfa, Dasatinib, Daunomycin Daunorubicin, Daunorubicin Hydrochloride, |
| | Daunorubicin Liposomal, DaunoXome ®, Decadron, Decitabine, Delta-Cortef ®, |
| | Deltasone ®, Denileukin, Diftitox, DepoCyt ™, Dexamethasone, Dexamethasone Acetate |
| | Dexamethasone Sodium Phosphate, Dexasone, Dexrazoxane, DHAD, DIC, Diodex |
| | Docetaxel, Doxil ®, Doxorubicin, Doxorubicin Liposomal, Droxia ™, DTIC, DTIC- |
| | Dome ®, Duralone ®, Efudex ®, Eligard ™, Ellence ™, Eloxatin ™, Elspar ®, Emcyt ®, |
| | Epirubicin, Epoetin Alfa, Erbitux, Erlotinib, Erwinia L-asparaginase, Estramustine, |
| | Ethyol Etopophos ®, Etoposide, Etoposide Phosphate, Eulexin ®, Everolimus, Evista ®, |
| | Exemestane, Fareston ®, Faslodex ®, Femara ®, Filgrastim, Floxuridine, Fludara ®, |
| | Fludarabine, Fluoroplex ®, Fluorouracil, Fluorouracil (cream), Fluoxymesterone, |
| | Flutamide, Folinic Acid, FUDR ®, Fulvestrant, G-CSF, Gefitinib, Gemcitabine, |
| | Gemtuzumab ozogamicin, Gemzar, Gleevec ™, Gliadel ® Wafer, GM-CSF, Goserelin, |
| | Granulocyte - Colony Stimulating Factor, Granulocyte Macrophage Colony Stimulating |
| | Factor, Halotestin ®, Herceptin ®, Hexadrol, Hexalen ®, Hexamethylmelamine, HMM, |
| | Hycamtin ®, Hydrea ®, Hydrocort Acetate ®, Hydrocortisone, Hydrocortisone Sodium |
| | Phosphate, Hydrocortisone Sodium Succinate, Hydrocortone Phosphate, Hydroxyurea, |
| | Ibritumomab, Ibritumomab, Tiuxetan, Idamycin ®, Idarubicin, Ifex ®, IFN-alpha, |
| | Ifosfamide, IL-11, IL-2, Imatinib mesylate, Imidazole Carboxamide, Interferon alfa, |
| | Interferon Alfa-2b (PEG Conjugate), Interleukin-2, Interleukin-11, Intron A ® |
| | (interferon alfa-2b), Iressa ®, Irinotecan, Isotretinoin, Ixabepilone, Ixempra ™, Kidrolase |
| | (t), Lanacort ®, Lapatinib, L-asparaginase, LCR, Lenalidomide, Letrozole, Leucovorin, |
| | Leukeran, Leukine ™, Leuprolide, Leurocristine, Leustatin ™, Liposomal Ara-C Liquid |
| | Pred ®, Lomustine, L-PAM, L-Sarcolysin, Lupron ®, Lupron Depot ®, Matulane ®, |
| | Maxidex, Mechlorethamine, Mechlorethamine Hydrochloride, Medralone ®, Medrol ®, |
| | Megace ®, Megestrol, Megestrol Acetate, Melphalan, Mercaptopurine, Mesna, |
| | Mesnex ™, Methotrexate, Methotrexate Sodium, Methylprednisolone, Meticorten ®, |
| | Mitomycin, Mitomycin-C, Mitoxantrone, M-Prednisol ®, MTC, MTX, Mustargen ®, |
| | Mustine, Mutamycin ®, Myleran ®, Mylocel ™, Mylotarg ®, Navelbine ®, Nelarabine, |
| | Neosar ®, Neulasta ™, Neumega ®, Neupogen ®, Nexavar ®, Nilandron ®, Nilutamide, |
| | Nipent ®, Nitrogen Mustard, Novaldex ®, Novantrone ®, Octreotide, Octreotide acetate, |
| | Oncospar ®, Oncovin ®, Ontak ®, Onxal ™, Oprevelkin, Orapred ®, Orasone ®, |
| | Oxaliplatin, Paclitaxel, Paclitaxel Protein-bound, Pamidronate, Panitumumab, |
| | Panretin ®, Paraplatin ®, Pediapred ®, PEG Interferon, Pegaspargase, Pegfilgrastim, |
| | PEG-INTRON ™, PEG-L-asparaginase, PEMETREXED, Pentostatin, Phenylalanine |
| | Mustard, Platinol ®, Platinol-AQ ®, Prednisolone, Prednisone, Prelone ®, Procarbazine, |
| | PROCRIT ®, Proleukin ®, Prolifeprospan 20 with Carmustine Implant, Purinethol ®, |
| | Raloxifene, Revlimid ®, Rheumatrex ®, Rituxan ®, Rituximab, Roferon-A ® (Interferon |
| | Alfa-2a), Rubex ®, Rubidomycin hydrochloride, Sandostatin ®, Sandostatin LAR ®, |
| | Sargramostim, Solu-Cortef ®, Solu-Medrol ®, Sorafenib, SPRYCEL ™, STI-571, |
| | Streptozocin, SU11248, Sunitinib, Sutent ®, Tamoxifen, Tarceva ®, Targretin ®, Taxol ®, |
| | Taxotere ®, Temodar ®, Temozolomide, Temsirolimus, Teniposide, TESPA, |
| | Thalidomide, Thalomid ®, TheraCys ®, Thioguanine, Thioguanine Tabloid ®, |
| | Thiophosphoamide, Thioplex ®, Thiotepa, TICE ®, Toposar ®, Topotecan, Toremifene, |
| | Torisel ®, Tositumomab, Trastuzumab, Treanda ®, Tretinoin, Trexall ™, Trisenox ®, |
| | TSPA, TYKERB ®, VCR, Vectibix ™, Velban ®, Velcade ®, VePesid ®, Vesanoid ®, |
| | Viadur ™, Vidaza ®, Vinblastine, Vinblastine Sulfate, Vincasar Pfs ®, Vincristine, |
| | Vinorelbine, Vinorelbine tartrate, VLB, VM-26, Vorinostat, VP-16, Vumon ®, Xeloda ®, |
| | Zanosar ®, Zevalin ™, Zinecard ®, Zoladex ®, Zoledronic acid, Zolinza, Zometa ® |
| Combination | CHOP (cyclophosphamide, doxorubicin, vincristine, and prednisone); CVP |
| Therapies | (cyclophosphamide, vincristine, and prednisone); RCVP (Rituximab + CVP); RCHOP |
| | (Rituximab + CHOP); RICE (Rituximab + ifosamide, carboplatin, etoposide); RDHAP, |
| | (Rituximab + dexamethasone, cytarabine, cisplatin); RESHAP (Rituximab + etoposide, |
| | methylprednisolone, cytarabine, cisplatin); combination treatment with vincristine, |
| | prednisone, and anthracycline, with or without asparaginase; combination treatment with |
| | daunorubicin, vincristine, prednisone, and asparaginase; combination treatment with |
| | teniposide and Ara-C (cytarabine); combination treatment with methotrexate and |
| | leucovorin; combination treatment with bleomycin, doxorubicin, etoposide, |
| | mechlorethamine, prednisone, vinblastine, and vincristine; FOLFOX4 regimen |
| | (oxaliplatin, leucovorin, and fluorouracil [5-FU]); FOLFIRI regimen (Irinotecan |
| | Hydrochloride, Fluorouracil, and Leucovorin Calcium); Levamisole regimen (5-FU and |
| | levamisole); NCCTG regimen (5-FU and low-dose leucovorin); NSABP regimen (5-FU |
| | and high-dose leucovorin); XAD (Xelox (Capecitabine + Oxaliplatin) + Bevacizumab + |
| | Dasatinib); FOLFOX/Bevacizumab/Hydroxychloroquine; German AIO regimen (folic |
| | acid, 5-FU, and irinotecan); Douillard regimen (folic acid, 5-FU, and irinotecan); |
| | CAPOX regimen (Capecitabine, oxaliplatin); FOLFOX6 regimen (oxaliplatin, |
| | leucovorin, and 5-FU); FOLFIRI regimen (folic acid, 5-FU, and irinotecan); FUFOX |
| | regimen (oxaliplatin, leucovorin, and 5-FU); FUOX regimen (oxaliplatin and 5-FU); |
| | IFL regimen (irinotecan, 5-FU, and leucovorin); XELOX regimen (capecitabine |
| | oxaliplatin); KHAD-L (ketoconazole, hydrocortisone, dutasteride and lapatinib); |
| Biologics | anti-CD52 antibodies (e.g., Alemtuzumab), anti-CD20 antibodies (e.g., Rituximab), |
| | anti-CD40 antibodies (e.g., SGN40) |
| Classes of | Anthracyclines and related substances, Anti-androgens, Anti-estrogens, Antigrowth |
| Treatments | hormones (e.g., Somatostatin analogs), Combination therapy (e.g., vincristine, bcnu, |
| | melphalan, cyclophosphamide, prednisone (VBMCP)), DNA methyltransferase |
| | inhibitors, Endocrine therapy - Enzyme inhibitor, Endocrine therapy - other hormone |
| | antagonists and related agents, Folic acid analogs (e.g., methotrexate), Folic acid |
| | analogs (e.g., pemetrexed), Gonadotropin releasing hormone analogs, Gonadotropin- |
| | releasing hormones, Monoclonal antibodies (EGFR-Targeted - e.g., panitumumab, |
| | cetuximab), Monoclonal antibodies (Her2-Targeted - e.g., trastuzumab), Monoclonal |
| | antibodies (Multi-Targeted - e.g., alemtuzumab), Other alkylating agents, Antineoplastic |
| | agents (e.g., asparaginase, ATRA, bexarotene, celecoxib, gemcitabine, hydroxyurea, |
| | irinotecan, topotecan, pentostatin), Cytotoxic antibiotics, Platinum compounds, |
| | Podophyllotoxin derivatives (e.g., etoposide), Progestogens, Protein kinase inhibitors |
| | (EGFR-Targeted), Protein kinase inhibitors (Her2 targeted therapy - e.g., lapatinib), |
| | Pyrimidine analogs (e.g., cytarabine), Pyrimidine analogs (e.g., fluoropyrimidines), |
| | Salicylic acid and derivatives (e.g., aspirin), Src-family protein tyrosine kinase inhibitors |
| | (e.g., dasatinib), Taxanes (e.g., nab-paclitaxel), Vinca Alkaloids and analogs, Vitamin D |
| | and analogs, Monoclonal antibodies (Multi-Targeted - e.g., bevacizumab), Protein |
| | kinase inhibitors (e.g., imatinib, sorafenib, sunitinib) |
| Prostate Cancer | Watchful waiting (i.e., monitor without treatment); Surgery (e.g., Pelvic |
| Treatments | lymphadenectomy, Radical prostatectomy, Transurethral resection of the prostate |
| | (TURP); Orchiectomy); Radiation therapy (e.g., external-beam radiation therapy |
| | (EBRT), Proton beam radiation; implantation of radioisotopes (i.e., iodine I 125, |
| | palladium, and iridium)); Hormone therapy (e.g., Luteinizing hormone-releasing |
| | hormone agonists such as leuprolide, goserelin, buserelin or ozarelix; Antiandrogens |
| | such as flutamide, 2-hydroxyflutamide, bicalutamide, megestrol acetate, nilutamide, |
| | ketoconazole, aminoglutethimide; calcitriol, gonadotropin-releasing hormone (GnRH), |
| | estrogens (DES, chlorotrianisene, ethinyl estradiol, conjugated estrogens USP, and DES- |
| | diphosphate), triptorelin, finasteride, cyproterone acetate, ASP3550); |
| | Cryosurgery/cryotherapy; Chemotherapy and Biologic therapy (dutasteride, zoledronate, |
| | azacitidine, docetaxel, prednisolone, celecoxib, atorvastatin, AMT2003, soy protein, |
| | LHRH agonist, PD-103, pomegranate extract, soy extract, taxotere, I-125, zoledronic |
| | acid, dasatinib, vitamin C, vitamin D, vitamin D3, vitamin E, gemcitabine, cisplatin, |
| | lenalidomide, prednisone, degarelix, OGX-011, OGX-427, MDV3100, tasquinimod, |
| | cabazitaxel, TOOKAD ®, lanreotide, PROSTVAC, GM-CSF, lenalidomide, samarium |
| | Sm-153 lexidronam, N-Methyl-D-Aspartate (NMDA)-Receptor Antagonist, sorafenib, |
| | sorafenib tosylate, mitoxantrone, ABI-008, hydrocortisone, panobinostat, soy-tomato |
| | extract, KHAD-L, TOK-001, cixutumumab, temsirolimus, ixabepilone, TAK-700, |
| | TAK-448, TRC105, cyclophosphamide, lenalidomide, MLN8237, GDC-0449, |
| | Alpharadin ®, ARN-509, PX-866, ISIS EIF4E Rx, AEZS-108, 131I-F16SIP Monoclonal |
| | Antibody, anti-OX40 antibody, Muscadine Plus, ODM-201, BBI608, ZD4054, erlotinib, |
| | rIL-2, epirubicin, estramustine phosphate, HuJ591-GS monoclonal (177Lu-J591), |
| | abraxane, IVIG, fermented wheat germ nutriment (FWGE), 153Sm-EDTMP, |
| | estramustine, mitoxantrone, vinblastine, carboplatin, paclitaxel, pazopanib, cytarabine, |
| | testosterone replacement, Zoledronic Acid, Strontium Chloride Sr 89, paricalcitol, |
| | satraplatin, RAD001 (everolimus), valproic acid, tea extract, Hamsa-1, |
| | hydroxychloroquine, sipuleucel-T, selenomethionine, selenium, lycopene, sunitinib, |
| | vandetanib, IMC-A12 antibody, monoclonal antibody IMC-3G3, ixabepilone, |
| | diindolylmethane, metformin, efavirenz, dasatinib, nilutamide, abiraterone, cabozantinib |
| | (XL184), isoflavines, cinacalcet hydrochloride, SB939, LY2523355, KX2-391, olaparib, |
| | genestein, digoxin, RO4929097, ipilimumab, bafetinib, cediranib maleate, MK2206, |
| | phenelzine sulfate, triptorelin pamoate, saracatinib, STA-9090, tesetaxel, pasireotide, |
| | afatinib, GTx 758, lonafarnib, satraplatin, radiolabeled antibody 7E11, |
| | FP253/fludarabine, Coxsackie A21 (CVA21) virus, ARRY-380, ARRY-382, anti- |
| | PSMA designer T cells, pemetrexed disodium, bortezomib, MDX-1106, white button |
| | mushroom extract, SU011248, MLN9708, BMTP-11, ABT-888, CX-4945, 4SC-205, |
| | temozolomide, MGAH22, vinorelbine ditartrate, Sodium Selenite, vorinostat, Ad- |
| | REIC/Dkk-3, ASG-5ME, IMF-001, PROHIBITIN-TP01, DSTP3086S, ridaforolimus, |
| | MK-2206, MK-0752, polyunsaturated fatty acids, I-125, statins, cholecalciferol, omega- |
| | 3 fatty acids, raloxifene, etoposide, POMELLA ™ extract, Lucrin depot); Cancer |
| | vaccines (e.g., DNA vaccines, peptide vaccines, dendritic cell vaccines, PEP223, |
| | PSA/TRICOM, PROSTVAC-V/TRICOM, PROSTVAC-F/TRICOM, PSA vaccine, |
| | TroVax ®, GI-6207, PSMA and TARP Peptide Vaccine); Ultrasound; Proton beam |
| | radiation |
| Colorectal Cancer | Primary Surgical Therapy (e.g., local excision; resection and anastomosis of primary |
| Treatments | lesion and removal of surrounding lymph nodes); Adjuvant Therapy (e.g., fluorouracil |
| | (5-FU), capecitabine, leucovorin, oxaliplatin, erlotinib, irinotecan, aspirin, mitomycin C, |
| | suntinib, cetuximab, bevacizumab, pegfilgrastim, panitumumab, ramucirumab, |
| | curcumin, celecoxib, FOLFOX4 regimen, FOLFOX6 regimen, FOLFIRI regimen, |
| | FUFOX regimen, FUOX regimen, IFL regimen, XELOX regimen, 5-FU and levamisole |
| | regimens, German AIO regimen, CAPOX regimen, Douillard regimen, XAD, RAD001 |
| | (everolimus), ARQ 197, BMS-908662, JI-101, hydroxychloroquine (HCQ), Yttrium |
| | Microspheres, EZN-2208, CS-7017, IMC-1121B, IMC-18F1, docetaxel, lonafarnib, |
| | Maytansinoid DM4-Conjugated Humanized Monoclonal Antibody huC242, paclitaxel, |
| | ARRY-380, ARRY-382, IMO-2055, MDX1105-01, CX-4945, Pazopanib, Ixabepilone, |
| | OSI-906, NPC-1C Chimeric Monoclonal Antibody, brivanib, Poly-ADP Ribose (PARP) |
| | Inhibitor, RO4929097, Anti-cancer vaccine, CEA vaccine, cyclophosphamide, yttrium |
| | Y 90 DOTA anti-CEA monoclonal antibody M5A, MEHD7945A, ABT-806, ABT-888, |
| | MEDI-565, LY2801653, AZD6244, PRI-724, BKM120, tivozanib, floxuridine, |
| | dexamethosone, NKTR-102, perifosine, regorafenib, EP0906, Celebrex, PHY906, |
| | KRN330, imatinib mesylate, azacitidine, entinostat, PX-866, ABX-EGF, BAY 43-9006, |
| | ESO-1 Lymphocytes and Aldesleukin, LBH589, olaparib, fostamatinib, PD 0332991, |
| | STA-9090, cholecalciferol, GI-4000, IL-12, AMG 706, temsirolimus, dulanermin, |
| | bortezomib, ursodiol, ridaforolimus, veliparib, NK012, Dalotuzumab, MK-2206, MK- |
| | 0752, lenalidomide, REOLYSIN ®, AUY922, PRI-724, BKM120, avastin, dasatinib); |
| | Adjuvant Radiation Therapy (particularly for rectal cancer) |

As shown in Table 8, cancer treatments include various surgical and therapeutic treatments. Anti-cancer agents include drugs such as small molecules and biologicals. The methods of the invention can be used to identify a biosignature comprising circulating biomarkers that can then be used for theranostic purposes such as monitoring a treatment efficacy, classifying a subject as a responder or non-responder to a treatment, or selecting a candidate therapeutic agent. The invention can be used to provide a theranosis for any cancer treatments, including without limitation themosis involving the cancer treatments in Tables 8-10. Cancer therapies that can be identified as candidate treatments by the methods of the invention include without limitation the chemotherapeutic agents listed in Tables 8-10 and any appropriate combinations thereof. In one embodiment, the treatments are specific for a specific type of cancer, such as the treatments listed for prostate cancer, colorectal cancer, breast cancer and lung cancer in Table 8. In other embodiments, the treatments are specific for a tumor regardless of its origin but that displays a certain biosignature, such as a biosignature comprising a marker listed in Tables 9-10.

The invention provides methods of monitoring a cancer treatment comprising identifying a series of biosignatures in a subject over a time course, such as before and after a treatment, or over time after the treatment. The biosignatures are compared to a reference to determine the efficacy of the treatment. In an embodiment, the treatment is selected from Tables 8-10, such as radiation, surgery, chemotherapy, biologic therapy, neo-adjuvant therapy, adjuvant therapy, or watchful waiting. The reference can be from another individual or group of individuals or from the same subject. For example, a subject with a biosignature indicative of a cancer pre-treatment may have a biosignature indicative of a healthy state after a successful treatment. Conversely, the subject may have a biosignature indicative of cancer after an unsuccessful treatment. The biosignatures can be compared over time to determine whether the subject's biosignatures indicate an improvement, worsening of the condition, or no change. Additional treatments may be called for if the cancer is worsening or there is no change over time. For example, hormone therapy may be used in addition to surgery or radiation therapy to treat more aggressive prostate cancers. One or more of the following miRs can be used in a biosignature for monitoring an efficacy of prostate cancer treatment: hsa-miR-1974, hsa-miR-27b, hsa-miR-103, hsa-miR-146a, hsa-miR-22, hsa-miR-382, hsa-miR-23a, hsa-miR-376c, hsa-miR-335, hsa-miR-142-5p, hsa-miR-221, hsa-miR-142-3p, hsa-miR-151-3p, hsa-miR-21, hsa-miR-16. One or more miRs listed in the following publication can be used in a biosignature for monitoring treatment of a cancer of the GI tract: Albulescu et al., Tissular and soluble miRNAs for diagnostic and therapy improvement in digestive tract cancers, Exp Rev Mol Diag, 11:1, 101-120.

In some embodiments, the invention provides a method of identifying a biosignature in a sample from a subject in order to select a candidate therapeutic. For example, the biosignature may indicate that a drug-associated target is mutated or differentially expressed, thereby indicating that the subject is likely to respond or not respond to certain treatments. The candidate treatments can be chosen from the anti-cancer agents or classes of therapeutic agents identified in Tables 8-10. In some embodiments, the candidate treatments identified according to the subject methods are chosen from at least the groups of treatments consisting of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, taxanes, temozolomide, toremifene, trastuzumab, VBMCP, and vincristine.

Similar to selecting a candidate treatment, the invention also provides a method of determining whether to treat a cancer at all. For example, prostate cancer can be a non-aggressive disease that is unlikely to substantially harm the subject. Radiation therapy with androgen ablation (hormone reduction) is the standard method of treating locally advanced prostate cancer. Morbidities of hormone therapy include impotence, hot flashes, and loss of libido. In addition, a treatment such as prostatectomy can have morbidities such as impotence or incontinence. Therefore, the invention provides biosignatures that indicate aggressiveness or a progression (e.g., stage or grade) of the cancer. A non-aggressive cancer or localized cancer might not require immediate treatment but rather be watched, e.g., “watchful waiting” of a prostate cancer. Whereas an aggressive or advanced stage lesion would require a concomitantly more aggressive treatment regimen.

Examples of biomarkers that can be detected, and treatment agents that can be selected or possibly avoided are listed in Table 9. For example, a biosignature is identified for a subject with a prostate cancer, wherein the biosignature comprises levels of androgen receptor (AR). Overexpression or overproduction of AR, such as high levels of mRNA levels or protein levels in a vesicle, provides an identification of candidate treatments for the subject. Such treatments include agents for treating the subject such as Bicalutamide, Flutamide, Leuprolide, or Goserelin. The subject is accordingly identified as a responder to Bicalutamide, Flutamide, Leuprolide, or Goserelin. In another illustrative example, BCRP mRNA, protein, or both is detected at high levels in a vesicle from a subject suffering from NSCLC. The subject may then be classified as a non-responder to the agents Cisplatin and Carboplatin, or the agents are considered to be less effective than other agents for treating NSCLC in the subject and not selected for use in treating the subject. Any of the following biomarkers can be assessed in a vesicle obtained from a subject, and the biomarker can be in the form including but not limited to one or more of a nucleic acid, polypeptide, peptide or peptide mimetic. In yet another illustrative example, a mutation in one or more of KRAS, BRAF, PIK3CA, and/or c-kit can be used to select a candidate treatment. For example, a mutation in KRAS or BRAF in a patient may indicate that cetuximab and/or panitumumab are likely to be less effective in treating the patient. Illustrative cancer lineages are indicated in the table as having known associations with the indicated agents. The lineages may be those from the National Comprehensive Cancer Network (NCCN) guidelines. The NCCN Compendium™ contains authoritative, scientifically derived information designed to support decision-making about the appropriate use of drugs and biologics in patients with cancer.

TABLE 9
Examples of Biomarkers, Lineage and Agents

| | | Possibly Less Effective | Possible Agents to |
|---|---|---|---|
| Biomarker | Cancer Lineage | Agents | Consider |
| AR (high expression) | Prostate | | Bicalutamide, Flutamide, |
| | | | Leuprolide, Goserelin |
| AR (high expression) | default | | Bicaluamide, Flutamide, |
| | | | Leuprolide, Goserelin |
| BCRP (high | Non-small cell lung cancer | Cisplatin, Carboplatin | |
| expression) | (NSCLC) | | |
| BCRP (low | Non-small cell lung cancer | | Cisplatin, Carboplatin |
| expression) | (NSCLC) | | |
| BCRP (high | default | Cisplatin, Carboplatin | |
| expression) | | | |
| BCRP (low | default | | Cisplatin, Carboplatin |
| expression) | | | |
| BRAF V600E | Colorectal | Cetuximab, Panitumumab | |
| (mutation positive) | | | |
| BRAF V600E | Colorectal | | Cetuximab, Panitumumab |
| (mutation negative) | | | |
| BRAF V600E | All other | Cetuximab, Panitumumab | |
| (mutation positive) | | | |
| BRAF V600E | All other | | Cetuximab, Panitumumab |
| (mutation negative) | | | |
| BRAF V600E | default | Cetuximab, Panitumumab | |
| (mutation positive) | | | |
| BRAF V600E | default | | Cetuximab, Panitumumab |
| (mutation negative) | | | |
| CD52 (high | Leukemia | | Alemtuzumab |
| expression) | | | |
| CD52 (low | Leukemia | Alemtuzumab | |
| expression) | | | |
| CD52 (high | default (Hematologic | | Alemtuzumab |
| expression) | malignancies only) | | |
| CD52 (low | default (Hematologic | Alemtuzumab | |
| expression) | malignancies only) | | |
| c-kit | Uveal Melanoma | | |
| c-kit (high expression) | Gastrointestinal Stromal | | Imatinib |
| | Tumors [GIST] | | |
| c-kit (high expression) | Extrahepatic Bile Duct | | Imatinib |
| | Tumors | | |
| c-kit (high expression) | Acute myeloid leukemia | | Imatinib |
| | (AML) | | |
| c-kit (high expression) | default | | Imatinib |
| EGFR (high copy | Head and neck squamous | | Erlotinib, Gefitinib |
| number) | cell carcinoma (HNSCC) | | |
| EGFR | Head and neck squamous | Erlotinib, Gefitinib | |
| | cell carcinoma (HNSCC) | | |
| EGFR (high copy | Non-small cell lung cancer | | Erlotinib, Gefitinib |
| number) | (NSCLC) | | |
| EGFR (low copy | Non-small cell lung cancer | Erlotinib, Gefitinib | |
| number) | (NSCLC) | | |
| EGFR (high copy | default | | Cetuxumab, Panitumumab, |
| number) | | | Erlotinib, Gefitinib |
| EGFR (low copy | default | Cetuxumab, Panitumumab, | |
| number) | | Erlotinib, Gefitinib | |
| ER (high expression) | Breast | Ixabepilone | Tamoxifen-based treatment, |
| | | | aromatase inhibitors |
| | | | (anastrazole, letrozole) |
| ER (low expression) | Breast | | Ixabepilone |
| ER (high expression) | Ovarian | | Tamoxifen-based treatment, |
| | | | aromatase inhibitors |
| | | | (anastrazole, letrozole) |
| ER (high expression) | default | | Tamoxifen-based treatment, |
| | | | aromatase inhibitors |
| | | | (anastrazole, letrozole) |
| ERCC1 (high | Non-small cell lung cancer | Carboplatin, Cisplatin | |
| expression) | (NSCLC) | | |
| ERCC1 (low | Non-small cell lung cancer | | Carboplatin, Cisplatin |
| expression) | (NSCLC) | | |
| ERCC1 (high | Small Cell Lung Cancer | Carboplatin, Cisplatin | |
| expression) | (SCLC) | | |
| ERCC1 (low | Small Cell Lung Cancer | | Carboplatin, Cisplatin |
| expression) | (SCLC) | | |
| ERCC1 (high | Gastric | Oxaliplatin | |
| expression) | | | |
| ERCC1 (low | Gastric | | Oxaliplatin |
| expression) | | | |
| ERCC1 (high | default | Carboplatin, Cisplatin, | |
| expression) | | Oxaliplatin | |
| ERCC1 (low | default | | Carboplatin, Cisplatin, |
| expression) | | | Oxaliplatin |
| HER-2 (high | Breast | | Lapatinib, Trastuzumab |
| expression) | | | |
| HER-2 (high | default | | Lapatinib, Trastuzumab |
| expression) | | | |
| KRAS (mutation | Colorectal cancer | Cetuximab, Panitumumab | |
| positive) | | | |
| KRAS (mutation | Colorectal cancer | | Cetuximab, Panitumumab |
| negative) | | | |
| KRAS (mutation | Non-small cell lung cancer | Erlotinib, Gefitinib | |
| positive) | (NSCLC) | | |
| KRAS (mutation | Non-small cell lung cancer | | Erlotinib, Gefitinib |
| negative) | (NSCLC) | | |
| KRAS (mutation | Bronchioloalveolar | Erlotinib | |
| positive) | carcinoma (BAC) or | | |
| | adenocarcinoma (BAC | | |
| | subtype) | | |
| KRAS (mutation | Bronchioloalveolar | | Erlotinib |
| negative) | carcinoma (BAC) or | | |
| | adenocarcinoma (BAC | | |
| | subtype) | | |
| KRAS (mutation | Multiple myeloma | VBMCP/Cyclophosphamide | |
| positive) | | | |
| KRAS (mutation | Multiple myeloma | | VBMCP/Cyclophosphamide |
| negative) | | | |
| KRAS (mutation | default | Cetuximab, Panitumumab | |
| positive) | | | |
| KRAS (mutation | default | | Cetuximab, panitumumab |
| negative) | | | |
| KRAS (mutation | default | Cetuximab, Erlotinib, | |
| positive) | | Panitumumab, Gefitinib | |
| KRAS (mutation | default | | Cetuximab, Erlotinib, |
| negative) | | | Panitumumab, Gefitinib |
| MGMT (high | Pituitary tumors, | Temozolomide | |
| expression) | oligodendroglioma | | |
| MGMT (low | Pituitary tumors, | | Temozolomide |
| expression) | oligodendroglioma | | |
| MGMT (high | Neuroendocrine tumors | Temozolomide | |
| expression) | | | |
| MGMT (low | Neuroendocrine tumors | | Temozolomide |
| expression) | | | |
| MGMT (high | default | Temozolomide | |
| expression) | | | |
| MGMT (low | default | | Temozolomide |
| expression) | | | |
| MRP1 (high | Breast | Cyclophosphamide | |
| expression) | | | |
| MRP1 (low | Breast | | Cyclophosphamide |
| expression) | | | |
| MRP1 (high | Small Cell Lung Cancer | Etoposide | |
| expression) | (SCLC) | | |
| MRP1 (low | Small Cell Lung Cancer | | Etoposide |
| expression) | (SCLC) | | |
| MRP1 (high | Nodal Diffuse Large B- | Cyclophosphamide/Vincristine | |
| expression) | Cell Lymphoma | | |
| MRP1 (low | Nodal Diffuse Large B- | | Cyclophosphamide/Vincristine |
| expression) | Cell Lymphoma | | |
| MRP1 (high | default | Cyclophosphamide, | |
| expression) | | Etoposide, Vincristine | |
| MRP1 (low | default | | Cyclophosphamide, |
| expression) | | | Etoposide, Vincristine |
| PDGFRA (high | Malignant Solitary Fibrous | | Imatinib |
| expression) | Tumor of the Pleura | | |
| | (MSFT) | | |
| PDGFRA (high | Gastrointestinal stromal | | Imatinib |
| expression) | tumor (GIST) | | |
| PDGFRA (high | Default | | Imatinib |
| expression) | | | |
| p-glycoprotein (high | Acute myeloid leukemia | Etoposide | |
| expression) | (AML) | | |
| p-glycoprotein (low | Acute myeloid leukemia | | Etoposide |
| expression) | (AML) | | |
| p-glycoprotein (high | Diffuse Large B-cell | Doxorubicin | |
| expression) | Lymphoma (DLBCL) | | |
| p-glycoprotein (low | Diffuse Large B-cell | | Doxorubicin |
| expression) | Lymphoma (DLBCL) | | |
| p-glycoprotein (high | Lung | Etoposide | |
| expression) | | | |
| p-glycoprotein (low | Lung | | Etoposide |
| expression) | | | |
| p-glycoprotein (high | Breast | Doxorubicin | |
| expression) | | | |
| p-glycoprotein (low | Breast | | Doxorubicin |
| expression) | | | |
| p-glycoprotein (high | Ovarian | Paclitaxel | |
| expression) | | | |
| p-glycoprotein (low | Ovarian | | Paclitaxel |
| expression) | | | |
| p-glycoprotein (high | Head and neck squamous | Vincristine | |
| expression) | cell carcinoma (HNSCC) | | |
| p-glycoprotein (low | Head and neck squamous | | Vincristine |
| expression) | cell carcinoma (HNSCC) | | |
| p-glycoprotein (high | default | Vincristine, Etoposide, | |
| expression) | | Doxorubicin, Paclitaxel | |
| p-glycoprotein (low | default | | Vincristine, Etoposide, |
| expression) | | | Doxorubicin, Paclitaxel |
| PR (high expression) | Breast | Chemoendocrine therapy | Tamoxifen, Anastrazole, |
| | | | Letrozole |
| PR (low expression) | default | Chemoendocrine therapy | Tamoxifen, Anastrazole, |
| | | | Letrozole |
| PTEN (high | Breast | | Trastuzumab |
| expression) | | | |
| PTEN (low | Breast | Trastuzumab | |
| expression) | | | |
| PTEN (high | Non-small cell Lung | | Gefitinib |
| expression) | Cancer (NSCLC) | | |
| PTEN (low | Non-small cell Lung | Gefitinib | |
| expression) | Cancer (NSCLC) | | |
| PTEN (high | Colorectal | | Cetuximab, Panitumumab |
| expression) | | | |
| PTEN (low | Colorectal | Cetuximab, Panitumumab | |
| expression) | | | |
| PTEN (high | Glioblastoma | | Erlotinib, Gefitinib |
| expression) | | | |
| PTEN (low | Glioblastoma | Erlotinib, Gefitinib | |
| expression) | | | |
| PTEN (high | default | | Cetuximab, Panitumumab, |
| expression) | | | Erlotinib, Gefitinib and |
| | | | Trastuzumab |
| PTEN (low | default | Cetuximab, Panitumumab, | |
| expression) | | Erlotinib, Gefitinib and | |
| | | Trastuzumab | |
| RRM1 (high | Non-small cell lung cancer | Gemcitabine | |
| experssion) | (NSCLC) | | |
| RRM1 (low | Non-small cell lung cancer | | Gemcitabine |
| expression) | (NSCLC) | | |
| RRM1 (high | Pancreas | Gemcitabine | |
| experssion) | | | |
| RRM1 (low | Pancreas | | Gemcitabine |
| expression) | | | |
| RRM1 (high | default | Gemcitabine | |
| experssion) | | | |
| RRM1 (low | default | | Gemcitabine |
| expression) | | | |
| SPARC (high | Breast | | nab-paclitaxel |
| expression) | | | |
| SPARC (high | default | | nab-paclitaxel |
| expression) | | | |
| TS (high expression) | Colorectal | fluoropyrimidines | |
| TS (low expression) | Colorectal | | fluoropyrimidines |
| TS (high expression) | Pancreas | fluoropyrimidines | |
| TS (low expression) | Pancreas | | fluoropyrimidines |
| TS (high expression) | Head and Neck Cancer | fluoropyrimidines | |
| TS (low expression) | Head and Neck Cancer | | fluoropyrimidines |
| TS (high expression) | Gastric | fluoropyrimidines | |
| TS (low expression) | Gastric | | fluoropyrimidines |
| TS (high expression) | Non-small cell lung cancer | fluoropyrimidines | |
| | (NSCLC) | | |
| TS (low expression) | Non-small cell lung cancer | | fluoropyrimidines |
| | (NSCLC) | | |
| TS (high expression) | Liver | fluoropyrimidines | |
| TS (low expression) | Liver | | fluoropyrimidines |
| TS (high expression) | default | fluoropyrimidines | |
| TS (low expression) | default | | fluoropyrimidines |
| TOPO1 (high | Colorectal | | Irinotecan |
| expression) | | | |
| TOPO1 (low | Colorectal | Irinotecan | |
| expression) | | | |
| TOPO1 (high | Ovarian | | Irinotecan |
| expression) | | | |
| TOPO1 (low | Ovarian | Irinotecan | |
| expression) | | | |
| TOPO1 (high | default | | Irinotecan |
| expression) | | | |
| TOPO1 (low | default | Irinotecan | |
| expression) | | | |
| TopoIIa (high | Breast | | Doxorubicin, liposomal- |
| epxression) | | | Doxorubicin, Epirubicin |
| TopoIIa (low | Breast | Doxorubicin, liposomal- | |
| expression) | | Doxorubicin, Epirubicin | |
| TopoIIa (high | default | | Doxorubicin, liposomal- |
| epxression) | | | Doxorubicin, Epirubicin |
| TopoIIa (low | default | Doxorubicin, liposomal- | |
| expression) | | Doxorubicin, Epirubicin | |

Other examples of biomarkers that can be detected and the treatment agents that can be selected or possibly avoided based on the biomarker signatures are listed in Table 10. For example, for a subject suffering from cancer, detecting overexpression of ADA in vesicles from a subject is used to classify the subject as a responder to pentostatin, or pentostatin identified as an agent to use for treating the subject. In another example, for a subject suffering from cancer, detecting overexpression of BCRP in vesicles from the subject is used to classify the subject as a non-responder to cisplatin, carboplatin, irinotecan, and topotecan, meaning that cisplatin, carboplatin, irinotecan, and topotecan are identified as agents that are suboptimal for treating the subject.

TABLE 10
Examples of Biomarkers, Agents and Resistance

| Gene Name | Expression Status | Candidate Agent(s) | Possible Resistance |
|---|---|---|---|
| ADA | Overexpressed | pentostatin | |
| ADA | Underexpressed | | cytarabine |
| AR | Overexpressed | abarelix, bicalutamide, | |
| | | flutamide, gonadorelin, | |
| | | goserelin, leuprolide | |
| ASNS | Underexpressed | asparaginase, pegaspargase | |
| BCRP (ABCG2) | Overexpressed | | cisplatin, carboplatin, |
| | | | irinotecan, topotecan |
| BRAF | Mutated | | panitumumab, |
| | | | cetuximmab |
| BRCA1 | Underexpressed | mitomycin | |
| BRCA2 | Underexpressed | mitomycin | |
| CD52 | Overexpressed | alemtuzumab | |
| CDA | Overexpressed | | cytarabine |
| c-erbB2 | High levels of | Trastuzumab, c-erbB2 | |
| | phosphorylation in | kinase inhibitor, lapatinib | |
| | epithelial cells | | |
| CES2 | Overexpressed | irinotecan | |
| c-kit | Overexpressed | sorafenib, sunitinib, | |
| | | imatinib | |
| COX-2 | Overexpressed | celecoxib | |
| DCK | Overexpressed | gemcitabine | cytarabine |
| DHFR | Underexpressed | methotrexate, pemetrexed | |
| DHFR | Overexpressed | | methotrexate |
| DNMT1 | Overexpressed | azacitidine, decitabine | |
| DNMT3A | Overexpressed | azacitidine, decitabine | |
| DNMT3B | Overexpressed | azacitidine, decitabine | |
| EGFR | Overexpressed | erlotinib, gefitinib, | |
| | | cetuximab, panitumumab | |
| EML4-ALK | Overexpressed (present) | petrexmed, crizotinib | |
| EPHA2 | Overexpressed | dasatinib | |
| ER | Overexpressed | anastrazole, exemestane, | |
| | | fulvestrant, letrozole, | |
| | | megestrol, tamoxifen, | |
| | | medroxyprogesterone, | |
| | | toremifene, | |
| | | aminoglutethimide | |
| ERCC1 | Overexpressed | | carboplatin, cisplatin, |
| | | | oxaliplatin |
| GART | Underexpressed | pemetrexed | |
| GRN (PCDGF, PGRN) | Overexpressed | | anti-oestrogen therapy, |
| | | | tamoxifen, faslodex, |
| | | | letrozole, herceptin in |
| | | | Her-2 overexpressing |
| | | | cells, doxorubicin |
| HER-2 (ERBB2) | Overexpressed | trastuzumab, lapatinib | |
| HIF-1α | Overexpressed | sorafenib, sunitinib, | |
| | | bevacizumab | |
| IκB-α | Overexpressed | bortezomib | |
| IGFBP3 | Underexpressed | letrozole | |
| IGFBP4 | Overexpressed | letrozole | |
| IGFBP5 | Underexpressed | letrozole | |
| Ki67 | Underexpressed | tamoxifen + chemotherapy | |
| KRAS | Mutated | | panitumumab, |
| | | | cetuximab |
| MET | Overexpressed | | gefitinib, erlotinib |
| MGMT | Underexpressed | temozolomide | |
| MGMT | Overexpressed | | temozolomide |
| MRP1 (ABCC1) | Overexpressed | | etoposide, paclitaxel, |
| | | | docetaxel, vinblastine, |
| | | | vinorelbine, topotecan, |
| | | | teniposide |
| P-gp (ABCB1) | Overexpressed | | doxorubicin, etoposide, |
| | | | epirubicin, paclitaxel, |
| | | | docetaxel, vinblastine, |
| | | | vinorelbine, topotecan, |
| | | | teniposide, liposomal |
| | | | doxorubicin |
| PDGFR-α | Overexpressed | sorafenib, sunitinib, | |
| | | imatinib | |
| PDGFR-β | Overexpressed | sorafenib, sunitinib, | |
| | | imatinib | |
| PIK3CA/PI3K | Mutation | | cetuximab, |
| | | | panitumumab, |
| | | | trastuzumab |
| PR | Overexpressed | exemestane, fulvestrant, | |
| | | gonadorelin, goserelin, | |
| | | medroxyprogesterone, | |
| | | megestrol, tamoxifen, | |
| | | toremifene | |
| PTEN | Underexpressed | | cetuximab, |
| | | | panitumumab, |
| | | | trastuzumab |
| RARA | Overexpressed | ATRA | |
| RRM1 | Underexpressed | gemcitabine, hydroxyurea | |
| RRM2 | Underexpressed | gemcitabine, hydroxyurea | |
| RRM2B | Underexpressed | gemcitabine, hydroxyurea | |
| RXR-α | Overexpressed | bexarotene | |
| RXR-β | Overexpressed | bexarotene | |
| SPARC | Overexpressed | nab-paclitaxel | |
| SRC | Overexpressed | dasatinib | |
| SSTR2 | Overexpressed | octreotide | |
| SSTR5 | Overexpressed | octreotide | |
| TLE3 | | | |
| TOPO I | Overexpressed | irinotecan, topotecan | |
| TOPO IIα | Overexpressed | doxorubicin, epirubicin, | |
| | | liposomal-doxorubicin | |
| TOPO IIβ | Overexpressed | doxorubicin, epirubicin, | |
| | | liposomal-doxorubicin | |
| TS | Underexpressed | capecitabine, 5- | |
| | | fluorouracil, pemetrexed | |
| TS | Overexpressed | | capecitabine, 5- |
| | | | fluorouracil |
| TUBB3 | Overexpressed | | paclitaxel, docetaxel |
| VDR | Overexpressed | calcitriol, cholecalciferol | |
| VEGFR1 (Flt1) | Overexpressed | sorafenib, sunitinib, | |
| | | bevacizumab | |
| VEGFR2 | Overexpressed | sorafenib, sunitinib, | |
| | | bevacizumab | |
| VHL | Underexpressed | sorafenib, sunitinib | |

Further drug associations and rules that are used in embodiments of the invention are found in U.S. patent application Ser. No. 12/658,770, filed Feb. 12, 2010; and International PCT Patent Applications PCT/US2010/000407, filed Feb. 11, 2010; PCT/US2010/54366, filed Oct. 27, 2010; PCT/US2011/067527, filed Dec. 28, 2011; and PCT/US2012/041393, filed Jun. 7, 2012, all of which applications are incorporated by reference herein in their entirety. See, e.g., “Table 4: Rules Summary for Treatment Selection” of PCT/US2010/54366; “Table 5: Rules Summary for Treatment Selection” of PCT/US2011/067527; and Tables 7-12 of PCT/US2012/041393.

Any drug-associated target can be part of a biosignature for providing a theranosis. A “druggable target” comprising a target that can be modulated with a therapeutic agent such as a small molecule or biologic, is a candidate for inclusion in the biosignature of the invention. Drug-associated targets also include biomarkers that can confer resistance to a treatment, such as shown in Tables 9 and 10. The biosignature can be based on either the gene, e.g., DNA sequence, and/or gene product, e.g., mRNA or protein, or the drug-associated target. Such nucleic acid and/or polypeptide can be profiled as applicable as to presence or absence, level or amount, activity, mutation, sequence, haplotype, rearrangement, copy number, or other measurable characteristic. The gene or gene product can be associated with a vesicle population, e.g., as a vesicle surface marker or as vesicle payload. In an embodiment, the invention provides a method of theranosing a cancer, comprising identifying a biosignature that comprises a presence or level of one or more drug-associated target, and selecting a candidate therapeutic based on the biosignature. The drug-associated target can be a circulating biomarker, a vesicle, or a vesicle associated biomarker. Because drug-associated targets can be independent of the tissue or cell-of-origin, biosignatures comprising drug-associated targets can be used to provide a theranosis for any proliferative disease, such as cancers from various anatomical origins, including cancers of unknown origin such as CUPS.

The drug-associated targets assessed using the methods of the invention comprise without limitation ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNA11, GNAQ, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, NRAS, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, PTPN12, RAF1, RARA, ROS1, RRM1, RRM2, RRM2B, RXRB, RXRG, SIK2, SPARC, SRC, SSTR1, SSTR2, SSTR3, SSTR4, SSTR5, Survivin, TK1, TLE3, TNF, TOP1, TOP2A, TOP2B, TS, TUBB3, TXN, TXNRD1, TYMS, VDR, VEGF, VEGFA, VEGFC, VHL, YES1, ZAP70, or any combination thereof. A biosignature including one or combination of these markers can be used to characterize a phenotype according to the invention, such as providing a theranosis. These markers are known to play a role in the efficacy of various chemotherapeutic agents against proliferative diseases. Accordingly, the markers can be assessed to select a candidate treatment for the cancer independent of the origin or type of cancer. In an embodiment, the invention provides a method of selecting a candidate therapeutic for a cancer, comprising identifying a biosignature comprising a level or presence of one or more drug associated target, and selecting the candidate therapeutic based on its predicted efficacy for a patient with the biosignature. The one or more drug-associated target can be one of the targets listed above, or in Tables 9-10. In some embodiments, at least 2, 3, 4, 5, 6, 7, 8, 9, 10, 12, 15, 20, 25, 30, 35, 40, 45, or at least 50 of the one or more drug-associated targets are assessed. The one or more drug-associated target can be associated with a vesicle, e.g., as a vesicle surface marker or as vesicle payload as either nucleic acid (e.g., DNA, mRNA) or protein. In some embodiments, the presence or level of a microRNA known to interact with the one or more drug-associated target is assessed, wherein a high level of microRNA known to suppress the one or more drug-associated target can indicate a lower expression of the one or more drug-associated target and thus a lower likelihood of response to a treatment against the drug-associated target. The one or more drug-associated target can be circulating biomarkers. The one or more drug-associated target can be assessed in a tissue sample. The predicted efficacy can be determined by comparing the presence or level of the one or more drug-associated target to a reference value, wherein a higher level that the reference indicates that the subject is a likely responder. The predicted efficacy can be determined using a classifier algorithm, wherein the classifier was trained by comparing the biosignature of the one or more drug-associated target in subjects that are known to be responders or non-responders to the candidate treatment. Molecular associations of the one or more drug-associated target with appropriate candidate targets are displayed in Tables 9-10 herein and U.S. patent application Ser. No. 12/658,770, filed Feb. 12, 2010; International PCT Patent Application PCT/US2010/000407, filed Feb. 11, 2010; International PCT Patent Application PCT/US2010/54366, filed Oct. 27, 2010; International Patent Application Serial No. PCT/US2011/031479, entitled “Circulating Biomarkers for Disease” and filed Apr. 6, 2011; International Patent Application Serial No. PCT/US2011/067527, entitled “MOLECULAR PROFILING OF CANCER” and filed Dec. 28, 2011; and U.S. Provisional Patent Application 61/427,788, filed Dec. 28, 2010; all of which applications are incorporated by reference herein in their entirety.

Table 11 of International Patent Application Serial No. PCT/US2011/031479, provides a listing of gene and corresponding protein symbols and names of many of the theranostic targets that are analyzed according to the methods of the invention. As understood by those of skill in the art, genes and proteins have developed a number of alternative names in the scientific literature. Thus, the listing in Table 11 of PCT/US2011/031479 and Table 2 of PCT/US2011/067527 comprise illustrative but not exhaustive compilations. A further listing of gene aliases and descriptions can be found using a variety of online databases, including GeneCards® (www.genecards.org), HUGO Gene Nomenclature (www.genenames.org), Entrez Gene (www.ncbi.nlm.nih.gov/entrez/query.fcgi?db=gene), UniProtKB/Swiss-Prot (www.uniprot.org), UniProtKB/TrEMBL (www.uniprot.org), OMIM (www.ncbi.nlm.nih.gov/entrez/query.fcgi?db=OMIM), GeneLoc (genecards.weizmann.ac.il/geneloc/), and Ensembl (www.ensembl.org). Generally, gene symbols and names below correspond to those approved by HUGO, and protein names are those recommended by UniProtKB/Swiss-Prot. Common alternatives are provided as well. Where a protein name indicates a precursor, the mature protein is also implied. Throughout the application, gene and protein symbols may be used interchangeably and the meaning can be derived from context as necessary.

As an illustration, a treatment can be selected for a subject suffering from Non-Small Cell Lung Cancer. One or more biomarkers, such as, but not limited to, EGFR, excision repair cross-complementation group 1 (ERCC1), p53, Ras, p27, class III beta tubulin, breast cancer gene 1 (BRCA1), breast cancer gene 1 (BRCA2), and ribonucleotide reductase messenger 1 (RRM1), can be assessed from a vesicle from the subject. Based on one or more characteristics of the one or more biomarkers, the subject can be determined to be a responder or non-responder for a treatment, such as, but not limited to, Erlotinib, Carboplatin, Paclitaxel, Gefitinib, or a combination thereof.

In another embodiment, a treatment can be selected for a subject suffering from Colorectal Cancer, and a biomarker, such as, but not limited to, K-ras, can be assessed from a vesicle from the subject. Based on one or more characteristics of the one or more biomarkers, the subject can be determined to be a responder or non-responder for a treatment, such as, but not limited to, Panitumumab, Cetuximab, or a combination thereof.

In another embodiment, a treatment can be selected for a subject suffering from Breast Cancer. One or more biomarkers, such as, but not limited to, HER2, toposiomerase II a, estrogen receptor, and progesterone receptor, can be assessed from a vesicle from the subject. Based on one or more characteristics of the one or more biomarkers, the subject can be determined to be a responder or non-responder for a treatment, such as, but not limited to, trastuzumab, anthracyclines, taxane, methotrexate, fluorouracil, or a combination thereof.

As described, the biosignature used to theranose a cancer can comprise analysis of one or more biomarker, which can be a protein or nucleic acid, including a mRNA or a microRNA. The biomarker can be detected in a bodily fluid and/or can be detected associated with a vesicle, e.g., as a vesicle antigen or as vesicle payload. In an illustrative example, the biosignature is used to identify a patient as a responder or non-responder to a tyrosine kinase inhibitor. The biomarkers can be one or more of those described in WO/2010/121238, entitled “METHODS AND KITS TO PREDICT THERAPEUTIC OUTCOME OF TYROSINE KINASE INHIBITORS” and filed Apr. 19, 2010; or WO/2009/105223, entitled “SYSTEMS AND METHODS OF CANCER STAGING AND TREATMENT” and filed Feb. 19, 2009; both of which applications are incorporated herein by reference in their entirety.

In an aspect, the present invention provides a method of determining whether a subject is likely to respond or not to a tyrosine kinase inhibitor, the method comprising identifying one or more biomarker in a vesicle population in a sample from the subject, wherein differential expression of the one or more biomarker in the sample as compared to a reference indicates that the subject is a responder or non-responder to the tyrosine kinase inhibitor. In an embodiment, the one or more biomarker comprises miR-497, wherein reduced expression of miR-497 indicates that the subject is a responder (i.e., sensitive to the tyrosine kinase inhibitor). In another embodiment, the one or more biomarker comprises onr or more of miR-21, miR-23a, miR-23b, and miR-29b, wherein upregulation of the microRNA indicates that the subject is a likely non-responder (i.e., resistant to the tyrosine kinase inhibitor). In some embodiments, the one or more biomarker comprises onr or more of hsa-miR-029a, hsa-let-7d, hsa-miR-100, hsa-miR-1260, hsa-miR-025, hsa-let-7i, hsa-miR-146a, hsa-miR-594-Pre, hsa-miR-024, FGFR1, MET, RAB25, EGFR, KIT and VEGFR2. In another embodiment, the one or more biomarker comprises FGF1, HOXC10 or LHFP, wherein higher expression of the biomarker indicates that the subject is a non-responder (i.e., resistant to the tyrosine kinase inhibitor). The method can be used to determine the sensitivity of a cancer to the tyrosine kinase inhibitor, e.g., a non-small cell lung cancer cell, kidney cancer or GIST. The tyrosine kinase inhibitor can be erlotinib, vandetanib, sunitinib and/or sorafenib, or other inhibitors that operate by a similar mechanism of action. A tyrosine kinase inhibitor includes any agent that inhibits the action of one or more tyrosine kinases in a specific or non-specific fashion. Tyrosine kinase inhibitors include small molecules, antibodies, peptides, or any appropriate entity that directly, indirectly, allosterically, or in any other way inhibits tyrosine residue phosphorylation. Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]q-uinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9, 12-epox-y-1H-d\ddot{υ}ndolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (commonly known as sunitinib), A-[A-[[4-chloro-3 (trifluoromethyl)phenyl]carbamoylamino]phenoxy]-N-methyl-pyridine-2-carboxamide (commonly known as sorafenib), EMD121974, and N-(3-ethynylphenyl)-6, 7-bis(2-methoxyethoxy)quinazolin-4-amine (commonly known as erlotinib). In some embodiments, the tyrosine kinase inhibitor has inhibitory activity upon the epidermal growth factor receptor (EGFR), VEGFR, PDGFR beta, and/or FLT3.

Thus, a treatment can be selected for the subject suffering from a cancer, based on a biosignature identified by the methods of the invention. Accordingly, the biosignature can comprise a presence or level of a circulating biomarker, including a microRNA, a vesicle, or any useful vesicle associated biomarker.

Biomarkers that can be used for theranosis of other diseases using the methods of the invention, including cardiovascular disease, neurological diseases and disorders, immune diseases and disorders and infectious disease, are described in International Patent Application Serial No. PCT/US2011/031479, entitled “Circulating Biomarkers for Disease” and filed Apr. 6, 2011, which application is incorporated by reference in its entirety herein.

### Biosignature Discovery

The systems and methods provided herein can be used in identifying a novel biosignature of a vesicle, such as one or more novel biomarkers for the diagnosis, prognosis or theranosis of a phenotype. In one embodiment, one or more vesicles can be isolated from a subject with a phenotype and a biosignature of the one or more vesicles determined. The biosignature can be compared to a subject without the phenotype. Differences between the two biosignatures can be determined and used to form a novel biosignature. The novel biosignature can then be used for identifying another subject as having the phenotype or not having the phenotype.

Differences between the biosignature from a subject with a particular phenotype can be compared to the biosignature from a subject without the particular phenotype. The one or more differences can be a difference in any characteristic of the vesicle. For example, the level or amount of vesicles in the sample, the half-life of the vesicle, the circulating half-life of the vesicle, the metabolic half-life of the vesicle, or the activity of the vesicle, or any combination thereof, can differ between the biosignature from the subject with a particular phenotype and the biosignature from the subject without the particular phenotype.

In some embodiments, one or more biomarkers differ between the biosignature from the subject with a particular phenotype and the biosignature from the subject without the particular phenotype. For example, the expression level, presence, absence, mutation, variant, copy number variation, truncation, duplication, modification, molecular association of one or more biomarkers, or any combination thereof, may differ between the biosignature from the subject with a particular phenotype and the biosignature from the subject without the particular phenotype. The biomarker can be any biomarker disclosed herein or that can be used to characterize a biological entity, including a circulating biomarker, such as protein or microRNA, a vesicle, or a component present in a vesicle or on the vesicle, such as any nucleic acid (e.g. RNA or DNA), protein, peptide, polypeptide, antigen, lipid, carbohydrate, or proteoglycan.

In an aspect, the invention provides a method of discovering a novel biosignature comprising comparing the biomarkers between two or more sample groups to identify biomarkers that show a difference between the sample groups. Multiple markers can be assessed in a panel format to potentially improve the performance of individual markers. In some embodiments, the multiple markers are assessed in a multiplex fashion. The ability of the individual markers and groups of markers to distinguish the groups can be assessed using statistical discriminate analysis or classification methods as used herein. Optimal panels of markers can be used as a biosignature to characterize the phenotype under analysis, such as to provide a diagnosis, prognosis or theranosis of a disease or condition. Optimization can be based on various criteria, including without limitation maximizing ROC AUC, accuracy, sensitivity at a certain specificity, or specificity at a certain sensitivity. The panels can include biomarkers from multiple types. For example, the biosignature can comprise vesicle antigens useful for capturing a vesicle population of interest, and the biosignature can further comprise payload markers within the vesicle population, including without limitation microRNAs, mRNAs, or soluble proteins. Optimal combinations can be identified as those vesicle antigens and payload markers with the greatest ROC AUC value when comparing two settings. As another example, the biosignature can be determined by assessing a vesicle population in addition to assessing circulating biomarkers that are not obtained by isolating exosomes, such as circulating proteins and/or microRNAs.

The phenotype can be any of those listed herein, e.g., in the “Phenotype” section above. For example, the phenotype can be a proliferative disorder such as a cancer or non-malignant growth, a perinatal or pregnancy related condition, an infectious disease, a neurological disorder, a cardiovascular disease, an inflammatory disease, an immune disease, or an autoimmune disease. The cancer includes without limitation lung cancer, non-small cell lung cancerm small cell lung cancer (including small cell carcinoma (oat cell cancer), mixed small cell/large cell carcinoma, and combined small cell carcinoma), colon cancer, breast cancer, prostate cancer, liver cancer, pancreatic cancer, brain cancer, kidney cancer, ovarian cancer, stomach cancer, melanoma, bone cancer, gastric cancer, breast cancer, glioma, glioblastoma, hepatocellular carcinoma, papillary renal carcinoma, head and neck squamous cell carcinoma, leukemia, lymphoma, myeloma, or other solid tumors.

Any of the types of biomarkers or specific biomarkers described herein can be assessed as part of a biosignature. Exemplary biomarkers include without limitation those in Tables 3, 4 and 5. The markers in the tables can be used for capture and/or detection of vesicles for characterizing phenotypes as disclosed herein. In some cases, multiple capture and/or detectors are used to enhance the characterization. The markers can be detected as protein or as mRNA, which can be circulating freely or in complex. The markers can be detected as vesicle surface antigens or and vesicle payload. The “Illustrate Class” indicates indications for which the markers are known markers. Those of skill will appreciate that the markers can also be used in alternate settings in certain instances. For example, a marker which can be used to characterize one type disease may also be used to characterize another disease.

Any of the types of biomarkers or specific biomarkers described herein can be assessed to discover a novel biosignature, e.g., the biomarkers in Tables 3-5. In an embodiment, the biomarkers selected for discovery comprise cell-specific biomarkers as listed herein, including without limitation the genes and microRNA listed in FIGS. 1-60 of International Patent Application Serial No. PCT/US2011/031479, entitled “Circulating Biomarkers for Disease” and filed Apr. 6, 2011, which application is incorporated by reference in its entirety herein, Tables 9-10 or Table 17. The biomarkers can comprise one or more disease associated, drug associated, or prognostic target such as listed in Table 11 or Table 12. The biomarkers can comprise one or more general vesicle marker, one or more cell-specific vesicle marker, and/or one or more disease-specific vesicle marker.

TABLE 11
Disease- and Drug-associated Biomarkers

| Gene | | Protein | |
|---|---|---|---|
| Symbol | Gene Name | Symbol | Protein Name |
| ABCB1, | ATP-binding cassette, sub-family B | ABCB1, | Multidrug resistance protein 1; P- |
| PGP | (MDR/TAP), member 1 | MDR1, PGP | glycoprotein |
| ABCC1, | ATP-binding cassette, sub-family C | MRP1, | Multidrug resistance-associated protein 1 |
| MRP1 | (CFTR/MRP), member 1 | ABCC1 | |
| ABCG2, | ATP-binding cassette, sub-family G | ABCG2 | ATP-binding cassette sub-family G member 2 |
| BCRP | (WHITE), member 2 | | |
| ACE2 | angiotensin I converting enzyme | ACE2 | Angiotensin-converting enzyme 2 precursor |
| | (peptidyl-dipeptidase A) 2 | | |
| ADA | adenosine deaminase | ADA | Adenosine deaminase |
| ADH1C | alcohol dehydrogenase 1C (class I), | ADH1G | Alcohol dehydrogenase 1C |
| | gamma polypeptide | | |
| ADH4 | alcohol dehydrogenase 4 (class II), pi | ADH4 | Alcohol dehydrogenase 4 |
| | polypeptide | | |
| AGT | angiotensinogen (serpin peptidase | ANGT, AGT | Angiotensinogen precursor |
| | inhibitor, clade A, member 8) | | |
| ALK | anaplastic lymphoma receptor tyrosine | ALK | ALK tyrosine kinase receptor precursor |
| | kinase | | |
| AR | androgen receptor | AR | Androgen receptor |
| AREG | amphiregulin | AREG | Amphiregulin precursor |
| ASNS | asparagine synthetase | ASNS | Asparagine synthetase [glutamine- |
| | | | hydrolyzing] |
| BCL2 | B-cell CLL/lymphoma 2 | BCL2 | Apoptosis regulator Bcl-2 |
| BDCA1, | CD1c molecule | CD1C | T-cell surface glycoprotein CD1c precursor |
| CD1C | | | |
| BIRC5 | baculoviral IAP repeat-containing 5 | BIRC5, | Baculoviral IAP repeat-containing protein 5; |
| | | Survivin | Survivin |
| BRAF | v-raf murine sarcoma viral oncogene | B-RAF, | Serine/threonine-protein kinase B-raf |
| | homolog B1 | BRAF | |
| BRCA1 | breast cancer 1, early onset | BRCA1 | Breast cancer type 1 susceptibility protein |
| BRCA2 | breast cancer 2, early onset | BRCA2 | Breast cancer type 2 susceptibility protein |
| CA2 | carbonic anhydrase II | CA2 | Carbonic anhydrase 2 |
| CAV1 | caveolin 1, caveolae protein, 22 kDa | CAV1 | Caveolin-1 |
| CCND1 | cyclin D1 | CCND1, | G1/S-specific cyclin-D1 |
| | | Cyclin D1, | |
| | | BCL-1 | |
| CD20, | membrane-spanning 4-domains, | CD20 | B-lymphocyte antigen CD20 |
| MS4A1 | subfamily A, member 1 | | |
| CD25, | interleukin 2 receptor, alpha | CD25 | Interleukin-2 receptor subunit alpha |
| IL2RA | | | precursor |
| CD33 | CD33 molecule | CD33 | Myeloid cell surface antigen CD33 |
| | | | precursor |
| CD52, | CD52 molecule | CD52 | CAMPATH-1 antigen precursor |
| CDW52 | | | |
| CDA | cytidine deaminase | CDA | Cytidine deaminase |
| CDH1, | cadherin 1, type 1, E-cadherin | E-Cad | Cadherin-1 precursor (E-cadherin) |
| ECAD | (epithelial) | | |
| CDK2 | cyclin-dependent kinase 2 | CDK2 | Cell division protein kinase 2 |
| CDKN1A, | cyclin-dependent kinase inhibitor 1A | CDKN1A, | Cyclin-dependent kinase inhibitor 1 |
| P21 | (p21, Cip1) | p21 | |
| CDKN1B | cyclin-dependent kinase inhibitor 1B | CDKN1B, | Cyclin-dependent kinase inhibitor 1B |
| | (p27, Kip1) | p27 | |
| CDKN2A, | cyclin-dependent kinase inhibitor 2A | CD21A, p16 | Cyclin-dependent kinase inhibitor 2A, |
| P16 | (melanoma, p16, inhibits CDK4) | | isoforms 1/2/3 |
| CES2 | carboxylesterase 2 (intestine, liver) | CES2, EST2 | Carboxylesterase 2 precursor |
| CK 5/6 | cytokeratin 5/cytokeratin 6 | CK 5/6 | Keratin, type II cytoskeletal 5; Keratin, type |
| | | | II cytoskeletal 6 |
| CK14, | keratin 14 | CK14 | Keratin, type I cytoskeletal 14 |
| KRT14 | | | |
| CK17, | keratin 17 | CK17 | Keratin, type I cytoskeletal 17 |
| KRT17 | | | |
| COX2, | prostaglandin-endoperoxide synthase 2 | COX-2, | Prostaglandin G/H synthase 2 precursor |
| PTGS2 | (prostaglandin G/H synthase and | PTGS2 | |
| | cyclooxygenase) | | |
| DCK | deoxycytidine kinase | DCK | Deoxycytidine kinase |
| DHFR | dihydrofolate reductase | DHFR | Dihydrofolate reductase |
| DNMT1 | DNA (cytosine-5-)-methyltransferase 1 | DNMT1 | DNA (cytosine-5)-methyltransferase 1 |
| DNMT3A | DNA (cytosine-5-)-methyltransferase 3 | DNMT3A | DNA (cytosine-5)-methyltransferase 3A |
| | alpha | | |
| DNMT3B | DNA (cytosine-5-)-methyltransferase 3 | DNMT3B | DNA (cytosine-5)-methyltransferase 3B |
| | beta | | |
| ECGF1, | thymidine phosphorylase | TYMP, PD- | Thymidine phosphorylase precursor |
| TYMP | | ECGF, | |
| | | ECDF1 | |
| EGFR, | epidermal growth factor receptor | EGFR, | Epidermal growth factor receptor precursor |
| ERBB1, | (erythroblastic leukemia viral (v-erb-b) | ERBB1, | |
| HER1 | oncogene homolog, avian) | HER1 | |
| EML4 | echinoderm microtubule associated | EML4 | Echinoderm microtubule-associated protein- |
| | protein like 4 | | like 4 |
| EPHA2 | EPH receptor A2 | EPHA2 | Ephrin type-A receptor 2 precursor |
| ER, ESR1 | estrogen receptor 1 | ER, ESR1 | Estrogen receptor |
| ERBB2, | v-erb-b2 erythroblastic leukemia viral | ERBB2, | Receptor tyrosine-protein kinase erbB-2 |
| HER2/NEU | oncogene homolog 2, neuro/glioblastoma | HER2, HER- | precursor |
| | derived oncogene homolog (avian) | 2/neu | |
| ERCC1 | excision repair cross-complementing | ERCC1 | DNA excision repair protein ERCC-1 |
| | rodent repair deficiency, | | |
| | complementation group 1 (includes | | |
| | overlapping antisense sequence) | | |
| ERCC3 | excision repair cross-complementing | ERCC3 | TFIIH basal transcription factor complex |
| | rodent repair deficiency, | | helicase XPB subunit |
| | complementation group 3 (xeroderma | | |
| | pigmentosum group B complementing) | | |
| EREG | Epiregulin | EREG | Proepiregulin precursor |
| FLT1 | fms-related tyrosine kinase 1 (vascular | FLT-1, | Vascular endothelial growth factor receptor |
| | endothelial growth factor/vascular | VEGFR1 | 1 precursor |
| | permeability factor receptor) | | |
| FOLR1 | folate receptor 1 (adult) | FOLR1 | Folate receptor alpha precursor |
| FOLR2 | folate receptor 2 (fetal) | FOLR2 | Folate receptor beta precursor |
| FSHB | follicle stimulating hormone, beta | FSHB | Follitropin subunit beta precursor |
| | polypeptide | | |
| FSHPRH1, | centromere protein I | FSHPRH1, | Centromere protein I |
| CENP1 | | CENP1 | |
| FSHR | follicle stimulating hormone receptor | FSHR | Follicle-stimulating hormone receptor |
| | | | precursor |
| FYN | FYN oncogene related to SRC, FGR, | FYN | Tyrosine-protein kinase Fyn |
| | YES | | |
| GART | phosphoribosylglycinamide | GART, PUR2 | Trifunctional purine biosynthetic protein |
| | formyltransferase, | | adenosine-3 |
| | phosphoribosylglycinamide synthetase, | | |
| | phosphoribosylaminoimidazole | | |
| | synthetase | | |
| GNA11, | guanine nucleotide binding protein (G | GNA11, G | Guanine nucleotide-binding protein subunit |
| GA11 | protein), alpha 11 (Gq class) | alpha-11, G- | alpha-11 |
| | | protein | |
| | | subunit alpha- | |
| | | 11 | |
| GNAQ, | guanine nucleotide binding protein (G | GNAQ | Guanine nucleotide-binding protein G(q) |
| GAQ | protein), q polypeptide | | subunit alpha |
| GNRH1 | gonadotropin-releasing hormone 1 | GNRH1, | Progonadoliberin-1 precursor |
| | (luteinizing-releasing hormone) | GON1 | |
| GNRHR1, | gonadotropin-releasing hormone | GNRHR1 | Gonadotropin-releasing hormone receptor |
| GNRHR | receptor | | |
| GSTP1 | glutathione S-transferase pi 1 | GSTP1 | Glutathione S-transferase P |
| HCK | hemopoietic cell kinase | HCK | Tyrosine-protein kinase HCK |
| HDAC1 | histone deacetylase 1 | HDAC1 | Histone deacetylase 1 |
| HGF | hepatocyte growth factor (hepapoietin A; | HGF | Hepatocyte growth factor precursor |
| | scatter factor) | | |
| HIF1A | hypoxia inducible factor 1, alpha subunit | HIF1A | Hypoxia-inducible factor 1-alpha |
| | (basic helix-loop-helix transcription | | |
| | factor) | | |
| HIG1, | HIG1 hypoxia inducible domain family, | HIG1, | HIG1 domain family member 1A |
| HIGD1A, | member 1A | HIGD1A, | |
| HIG1A | | HIG1A | |
| HSP90AA1, | heat shock protein 90 kDa alpha | HSP90, | Heat shock protein HSP 90-alpha |
| HSP90, | (cytosolic), class A member 1 | HSP90A | |
| HSPCA | | | |
| IGF1R | insulin-like growth factor 1 receptor | IGF-1R | Insulin-like growth factor 1 receptor |
| | | | precursor |
| IGFBP3, | insulin-like growth factor binding protein 3 | IGFBP-3, | Insulin-like growth factor-binding protein 3 |
| IGFRBP3 | | IBP-3 | precursor |
| IGFBP4, | insulin-like growth factor binding protein 4 | IGFBP-4, | Insulin-like growth factor-binding protein 4 |
| IGFRBP4 | | IBP-4 | precursor |
| IGFBP5, | insulin-like growth factor binding protein 5 | IGFBP-5, | Insulin-like growth factor-binding protein 5 |
| IGFRBP5 | | IBP-5 | precursor |
| IL13RA1 | interleukin 13 receptor, alpha 1 | IL-13RA1 | Interleukin-13 receptor subunit alpha-1 |
| | | | precursor |
| KDR | kinase insert domain receptor (a type III | KDR, | Vascular endothelial growth factor receptor |
| | receptor tyrosine kinase) | VEGFR2 | 2 precursor |
| KIT, c-KIT | v-kit Hardy-Zuckerman 4 feline sarcoma | KIT, c-KIT, | Mast/stem cell growth factor receptor |
| | viral oncogene homolog | CD117, SCFR | precursor |
| KRAS | v-Ki-ras2 Kirsten rat sarcoma viral | K-RAS | GTPase KRas precursor |
| | oncogene homolog | | |
| LCK | lymphocyte-specific protein tyrosine | LCK | Tyrosine-protein kinase Lck |
| | kinase | | |
| LTB | lymphotoxin beta (TNF superfamily, | LTB, TNF3 | Lymphotoxin-beta |
| | member 3) | | |
| LTBR | lymphotoxin beta receptor (TNFR | LTBR, | Tumor necrosis factor receptor superfamily |
| | superfamily, member 3) | LTBR3, | member 3 precursor |
| | | TNFR | |
| LYN | v-yes-1 Yamaguchi sarcoma viral related | LYN | Tyrosine-protein kinase Lyn |
| | oncogene homolog | | |
| MET, c-MET | met proto-oncogene (hepatocyte growth | MET, c-MET | Hepatocyte growth factor receptor precursor |
| | factor receptor) | | |
| MGMT | O-6-methylguanine-DNA | MGMT | Methylated-DNA--protein-cysteine |
| | methyltransferase | | methyltransferase |
| MKI67, KI67 | antigen identified by monoclonal | Ki67, Ki-67 | Antigen KI-67 |
| | antibody Ki-67 | | |
| MLH1 | mutL homolog 1, colon cancer, | MLH1 | DNA mismatch repair protein Mlh1 |
| | nonpolyposis type 2 (E. coli) | | |
| MMR | mismatch repair (refers to MLH1, | | |
| | MSH2, MSH5) | | |
| MSH2 | mutS homolog 2, colon cancer, | MSH2 | DNA mismatch repair protein Msh2 |
| | nonpolyposis type 1 (E. coli) | | |
| MSH5 | mutS homolog 5 (E. coli) | MSH5, | MutS protein homolog 5 |
| | | hMSH5 | |
| MYC, c- | v-myc myelocytomatosis viral oncogene | MYC, c-MYC | Myc proto-oncogene protein |
| MYC | homolog (avian) | | |
| NBN, P95 | nibrin | NBN, p95 | Nibrin |
| NDGR1 | N-myc downstream regulated 1 | NDGR1 | Protein NDGR1 |
| NFKB1 | nuclear factor of kappa light polypeptide | NFKB1 | Nuclear factor NF-kappa-B p105 subunit |
| | gene enhancer in B-cells 1 | | |
| NFKB2 | nuclear factor of kappa light polypeptide | NFKB2 | Nuclear factor NF-kappa-B p100 subunit |
| | gene enhancer in B-cells 2 (p49/p100) | | |
| NFKBIA | nuclear factor of kappa light polypeptide | NFKBIA | NF-kappa-B inhibitor alpha |
| | gene enhancer in B-cells inhibitor, alpha | | |
| NRAS | neuroblastoma RAS viral (v-ras) | NRAS | GTPase NRas, Transforming protein N-Ras |
| | oncogene homolog | | |
| ODC1 | ornithine decarboxylase 1 | ODC | Ornithine decarboxylase |
| OGFR | opioid growth factor receptor | OGFR | Opioid growth factor receptor |
| PARP1 | poly (ADP-ribose) polymerase 1 | PARP-1 | Poly [ADP-ribose] polymerase 1 |
| PDGFC | platelet derived growth factor C | PDGF-C, | Platelet-derived growth factor C precursor |
| | | VEGF-E | |
| PDGFR | platelet-derived growth factor receptor | PDGFR | Platelet-derived growth factor receptor |
| PDGFRA | platelet-derived growth factor receptor, | PDGFRA, | Alpha-type platelet-derived growth factor |
| | alpha polypeptide | PDGFR2, | receptor precursor |
| | | CD140 A | |
| PDGFRB | platelet-derived growth factor receptor, | PDGFRB, | Beta-type platelet-derived growth factor |
| | beta polypeptide | PDGFR, | receptor precursor |
| | | PDGFR1, | |
| | | CD140 B | |
| PGR | progesterone receptor | PR | Progesterone receptor |
| PIK3CA | phosphoinositide-3-kinase, catalytic, | PI3K subunit | phosphoinositide-3-kinase, catalytic, alpha |
| | alpha polypeptide | p110α | polypeptide |
| POLA1 | polymerase (DNA directed), alpha 1, | POLA, | DNA polymerase alpha catalytic subunit |
| | catalytic subunit; polymerase (DNA | POLA1, p180 | |
| | directed), alpha, polymerase (DNA | | |
| | directed), alpha 1 | | |
| PPARG, | peroxisome proliferator-activated | PPARG | Peroxisome proliferator-activated receptor |
| PPARG1, | receptor gamma | | gamma |
| PPARG2, | | | |
| PPAR- | | | |
| gamma, | | | |
| NR1C3 | | | |
| PPARGC1A, | peroxisome proliferator-activated | PGC-1-alpha, | Peroxisome proliferator-activated receptor |
| LEM6, | receptor gamma, coactivator 1 alpha | PPARGC-1- | gamma coactivator 1-alpha; PPAR-gamma |
| PGC1, | | alpha | coactivator 1-alpha |
| PGC1A, | | | |
| PPARGC1 | | | |
| PSMD9, P27 | proteasome (prosome, macropain) 26S | p27 | 26S proteasome non-ATPase regulatory |
| | subunit, non-ATPase, 9 | | subunit 9 |
| PTEN, | phosphatase and tensin homolog | PTEN | Phosphatidylinositol-3,4,5-trisphosphate 3- |
| MMAC1, | | | phosphatase and dual-specificity protein |
| TEP1 | | | phosphatase; Mutated in multiple advanced |
| | | | cancers 1 |
| PTPN12 | protein tyrosine phosphatase, non- | PTPG1 | Tyrosine-protein phosphatase non-receptor |
| | receptor type 12 | | type 12; Protein-tyrosine phosphatase G1 |
| RAF1 | v-raf-1 murine leukemia viral oncogene | RAF, RAF-1, | RAF proto-oncogene serine/threonine- |
| | homolog 1 | c-RAF | protein kinase |
| RARA | retinoic acid receptor, alpha | RAR, RAR- | Retinoic acid receptor alpha |
| | | alpha, RARA | |
| ROS1, ROS, | c-ros oncogene 1, receptor tyrosine | ROS1, ROS | Proto-oncogene tyrosine-protein kinase ROS |
| MCF3 | kinase | | |
| RRM1 | ribonucleotide reductase M1 | RRM1, RR1 | Ribonucleoside-diphosphate reductase large |
| | | | subunit |
| RRM2 | ribonucleotide reductase M2 | RRM2, | Ribonucleoside-diphosphate reductase |
| | | RR2M, RR2 | subunit M2 |
| RRM2B | ribonucleotide reductase M2 B (TP53 | RRM2B, | Ribonucleoside-diphosphate reductase |
| | inducible) | P53R2 | subunit M2 B |
| RXRB | retinoid X receptor, beta | RXRB | Retinoic acid receptor RXR-beta |
| RXRG | retinoid X receptor, gamma | RXRG, | Retinoic acid receptor RXR-gamma |
| | | RXRC | |
| SIK2 | salt-inducible kinase 2 | SIK2, | Salt-inducible protein kinase 2; |
| | | Q9H0K1 | Serine/threonine-protein kinase SIK2 |
| SLC29A1 | solute carrier family 29 (nucleoside | ENT-1 | Equilibrative nucleoside transporter 1 |
| | transporters), member 1 | | |
| SPARC | secreted protein, acidic, cysteine-rich | SPARC | SPARC precursor; Osteonectin |
| | (osteonectin) | | |
| SRC | v-src sarcoma (Schmidt-Ruppin A-2) | SRC | Proto-oncogene tyrosine-protein kinase Src |
| | viral oncogene homolog (avian) | | |
| SSTR1 | somatostatin receptor 1 | SSTR1, | Somatostatin receptor type 1 |
| | | SSR1, SS1R | |
| SSTR2 | somatostatin receptor 2 | SSTR2, | Somatostatin receptor type 2 |
| | | SSR2, SS2R | |
| SSTR3 | somatostatin receptor 3 | SSTR3, | Somatostatin receptor type 3 |
| | | SSR3, SS3R | |
| SSTR4 | somatostatin receptor 4 | SSTR4, | Somatostatin receptor type 4 |
| | | SSR4, SS4R | |
| SSTR5 | somatostatin receptor 5 | SSTR5, | Somatostatin receptor type 5 |
| | | SSR5, SS5R | |
| TK1 | thymidine kinase 1, soluble | TK1, KITH | Thymidine kinase, cytosolic |
| TLE3 | transducin-like enhancer of split 3 | TLE3 | Transducin-like enhancer protein 3 |
| | (E(sp1) homolog, Drosophila) | | |
| TNF | tumor necrosis factor (TNF superfamily, | TNF, TNF- | Tumor necrosis factor precursor |
| | member 2) | alpha, TNF-a | |
| TOP1, | topoisomerase (DNA) I | TOP1, | DNA topoisomerase 1 |
| TOPO1 | | TOPO1 | |
| TOP2A, | topoisomerase (DNA) II alpha 170 kDa | TOP2A, | DNA topoisomerase 2-alpha; Topoisomerase |
| TOPO2A | | TOP2, | II alpha |
| | | TOPO2A | |
| TOP2B, | topoisomerase (DNA) II beta 180 kDa | TOP2B, | DNA topoisomerase 2-beta; Topoisomerase |
| TOPO2B | | TOPO2B | II beta |
| TP53 | tumor protein p53 | p53 | Cellular tumor antigen p53 |
| TUBB3 | tubulin, beta 3 | Beta III | Tubulin beta-3 chain |
| | | tubulin, | |
| | | TUBB3, | |
| | | TUBB4 | |
| TXN | thioredoxin | TXN, TRX, | Thioredoxin |
| | | TRX-1 | |
| TXNRD1 | thioredoxin reductase 1 | TXNRD1, | Thioredoxin reductase 1, cytoplasmic; |
| | | TXNR | Oxidoreductase |
| TYMS, TS | thymidylate synthetase | TYMS, TS | Thymidylate synthase |
| VDR | vitamin D (1,25-dihydroxyvitamin D3) | VDR | Vitamin D3 receptor |
| | receptor | | |
| VEGFA, | vascular endothelial growth factor A | VEGF-A, | Vascular endothelial growth factor A |
| VEGF | | VEGF | precursor |
| VEGFC | vascular endothelial growth factor C | VEGF-C | Vascular endothelial growth factor C |
| | | | precursor |
| VHL | von Hippel-Lindau tumor suppressor | VHL | Von Hippel-Lindau disease tumor |
| | | | suppressor |
| YES1 | v-yes-1 Yamaguchi sarcoma viral | YES1, Yes, | Proto-oncogene tyrosine-protein kinase Yes |
| | oncogene homolog 1 | p61-Yes | |
| ZAP70 | zeta-chain (TCR) associated protein | ZAP-70 | Tyrosine-protein kinase ZAP-70 |
| | kinase 70 kDa | | |

The biomarkers used for biosignature discovery can comprise include markers commonly associated with vesicles, including without limitation one or more vesicle biomarker in Table 3, 4 or 5. Other biomarkers can be selected from those disclosed in the ExoCarta database, available at exocarta.ludwig.edu.au, which discloses proteins and RNA molecules identified in vesicles. See also Mathivanan and Simpson, ExoCarta: A compendium of exosomal proteins and RNA. Proteomics. 2009 Nov. 9(21):4997-5000.

The biomarkers used for biosignature discovery can comprise include markers commonly associated with vesicles, including without limitation one or more of A33, a33 n15, AFP, ALA, ALIX, ALP, AnnexinV, APC, ASCA, ASPH (246-260), ASPH (666-680), ASPH (A-10), ASPH (D01P), ASPH (D03), ASPH (G-20), ASPH (H-300), AURKA, AURKB, B7H3, B7H4, BCA-225, BCNP, BCNP1, BDNF, BRCA, CA125 (MUC16), CA-19-9, C-Bir, CD1.1, CD10, CD174 (Lewis y), CD24, CD44, CD46, CD59 (MEM-43), CD63, CD66e CEA, CD73, CD81, CD9, CDA, CDAC1 1a2, CEA, C-Erb2, C-erbB2, CRMP-2, CRP, CXCL12, CYFRA21-1, DLL4, DR3, EGFR, Epcam, EphA2, EphA2 (H-77), ER, ErbB4, EZH2, FASL, FRT, FRT c.f23, GDF15, GPCR, GPR30, Gro-alpha, HAP, HBD 1, HBD2, HER 3 (ErbB3), HSP, HSP70, hVEGFR2, iC3b, IL 6 Unc, IL-1B, IL6 Unc, IL6R, IL8, IL-8, INSIG-2, KLK2, L1CAM, LAMN, LDH, MACC-1, MAPK4, MART-1, MCP-1, M-CSF, MFG-E8, MIC1, MIF, MIS RH, MMG, MMP26, MMP7, MMP9, MS4A1, MUC1, MUC1 seq1, MUC1 seq11A, MUC17, MUC2, Ncam, NGAL, NPGP/NPFF2, OPG, OPN, p53, p53, PA2G4, PBP, PCSA, PDGFRB, PGP9.5, PIM1, PR (B), PRL, PSA, PSMA, PSME3, PTEN, R5-CD9 Tube 1, Reg IV, RUNX2, SCRN1, seprase, SERPINB3, SPARC, SPB, SPDEF, SRVN, STAT 3, STEAP1, TF (FL-295), TFF3, TGM2, TIMP-1, TIMP1, TIMP2, TMEM211, TMPRSS2, TNF-alpha, Trail-R2, Trail-R4, TrKB, TROP2, Tsg 101, TWEAK, UNC93A, VEGF A, and YPSMA-1. The biomarkers can include one or more of NSE, TRIM29, CD63, CD151, ASPH, LAMP2, TSPAN1, SNA1L, CD45, CKS1, NSE, FSHR, OPN, FTH1, PGP9, ANNEXIN 1, SPD, CD81, EPCAM, PTH1R, CEA, CYTO 7, CCL2, SPA, KRAS, TWIST1, AURKB, MMP9, P27, MMP1, HLA, HIF, CEACAM, CENPH, BTUB, INTG b4, EGFR, NACC1, CYTO 18, NAP2, CYTO 19, ANNEXIN V, TGM2, ERB2, BRCA1, B7H3, SFTPC, PNT, NCAM, MS4A1, P53, INGA3, MUC2, SPA, OPN, CD63, CD9, MUC1, UNCR3, PAN ADH, HCG, TIMP, PSMA, GPCR, RACK1, PCSA, VEGF, BMP2, CD81, CRP, PRO GRP, B7H3, MUC1, M2PK, CD9, PCSA, and PSMA. The biomarkers can also include one or more of TFF3, MS4A1, EphA2, GAL3, EGFR, N-gal, PCSA, CD63, MUC1, TGM2, CD81, DR3, MACC-1, TrKB, CD24, TIMP-1, A33, CD66 CEA, PRL, MMP9, MMP7, TMEM211, SCRN1, TROP2, TWEAK, CDACC1, UNC93A, APC, C-Erb, CD10, BDNF, FRT, GPR30, P53, SPR, OPN, MUC2, GRO-1, tsg 101 and GDF15. In embodiments, the biomarkers used to discover a biosignature comprise one or more of those shown in FIGS. 99, 100, 108A-C, 114A, and/or 115A-E of International Patent Application Serial No. PCT/US2011/031479, entitled “Circulating Biomarkers for Disease” and filed Apr. 6, 2011, which application is incorporated by reference in its entirety herein.

The markers can include one or more of NY-ESO-1, SSX-2, SSX-4, XAGE-1b, AMACR, p90 autoantigen, LEDGF. See Xie et al., Journal of Translational Medicine 2011, 9:43, which publication is incorporated by reference in its entirety herein. The markers can include one or more of STEAP and EZH2. See Hayashi et al., Journal of Translational Medicine 2011, 9:191, which publication is incorporated by reference in its entirety herein. The markers can include one or more members of the miR-183-96-182 cluster (miRs-183, 96 and 182, which are expressed as a cluster and share sequence similarity) or a zinc transporter, such as hZIP1. See Mihelich et al., The miR-183-96-182 cluster is overexpressed in prostate tissue and regulates zinc homeostasis in prostate cells. J Biol Chem. 2011 Nov. 1. [Epub ahead of print], which publication is incorporated by reference in its entirety herein.

The markers can include one or more of RAD23B, FBP1, TNFRSF1A, CCNG2, NOTCH3, ETV1, BID, SIM2, LETMD1, ANXA1, miR-519d, and miR-647. The markers can include one or more of RAD23B, FBP1, TNFRSF1A, NOTCH3, ETV1, BID, SIM2, ANXA1 and BCL2. See Long et al., Am J Pathol. 2011 July; 179(1):46-54, which publication is incorporated by reference in its entirety herein. The markers can include one or more of ANPEP, ABL1, PSCA, EFNA1, HSPB1, INMT and TRIP13. See Larkin et al, British Journal of Cancer (2011), 1-9. These markers can be assessed as RNA or protein. In an embodiment, one or more of these markers are used predict recurrence or prostate cancer. In another embodiment, ANPEP and ABL1 or ANPEP and PSCA are assessed to predict aggressiveness.

One of skill will appreciate that any marker disclosed herein or that can be compared between two samples or sample groups of interest can be used to discover a novel biosignature for any given biological setting that can be compared, e.g., healthy versus diseased, late stage versus early stage disease, drug responder versus non-responder, disease 1 versus disease 2, and the like. Markers, such as one or more marker disclosed herein such as in Tables 3, 4, 5 or 11, can then be chosen individually or as a panel to form a biosignature that can be used to characterize the phenotype.

The one or more differences can then be used to form a candidate biosignature for the particular phenotype, such as the diagnosis of a condition, diagnosis of a stage of a disease or condition, prognosis of a condition, or theranosis of a condition. The novel biosignature can then be used to identify the phenotype in other subjects. The biosignature of a vesicle for a new subject can be determined and compared to the novel signature to determine if the subject has the particular phenotype for which the novel biosignature was identified from.

For example, the biosignature of a subject with cancer can be compared to another subject without cancer. Any differences can be used to form a novel biosignature for the diagnosis of the cancer. In another embodiment, the biosignature of a subject with an advanced stage of cancer can be compared to another subject with a less advanced stage of cancer. Any differences can be used to form a novel biosignature for the classification of the stage of cancer. In yet another embodiment, the biosignature of a subject with an advanced stage of cancer can be compared to another subject with a less advanced stage of cancer. Any differences can be used to form a novel biosignature for the classification of the stage of cancer.

In one embodiment, the phenotype is drug resistance or non-responsiveness to a therapeutic. One or more vesicles can be isolated from a non-responder to a particular treatment and the biosignature of the vesicle determined. The biosignature of the vesicle obtained from the non-responsder can be compared to the biosignature of a vesicle obtained from a responsder. Differences between the biosignature from the non-responder can be compared to the biosignature from the responder. The one or more differences can be a difference in any characteristic of the vesicle. For example, the level or amount of vesicles in the sample, the half-life of the vesicle, the circulating half-life of the vesicle, the metabolic half-life of the vesicle, the activity of the vesicle, or any combination thereof, can differ between the biosignature from the non-responder and the biosignature from the responder.

In some embodiments, one or more biomarkers differ between the biosignature from the non-responder and the biosignature from the responder. For example, the expression level, presence, absence, mutation, variant, copy number variation, truncation, duplication, modification, molecular association of one or more biomarkers, or any combination thereof, may differ between the biosignature from the non-responder and the biosignature from the responder.

In some embodiments, the difference can be in the amount of drug or drug metabolite present in the vesicle. Both the responder and non-responder can be treated with a therapeutic. A comparison between the biosignature from the responder and the biosignature from the non-responder can be performed, the amount of drug or drug metabolite present in the vesicle from the responder differs from the amount of drug or drug metabolite present in the non-responder. The difference can also be in the half-life of the drug or drug metabolite. A difference in the amount or half-life of the drug or drug metabolite can be used to form a novel biosignature for identifying non-responders and responders.

A vesicle useful for methods and compositions described herein can be discovered by taking advantage of its physicochemical characteristics. For example, a vesicle can be discovered by its size, e.g., by filtering biological matter in a known range from 30-120 nm in diameter. Size-based discovery methods, such as differential centrifugation, sucrose gradient centrifugation, or filtration have been used for isolation of a vesicle.

A vesicle can be discovered by its molecular components. Molecular property-based discovery methods include, but are not limited to, immunological isolation using antibodies recognizing molecules associated with vesicle. For example, a surface molecule associated with a vesicle includes, but not limited to, a MHC-II molecule, CD63, CD81, LAMP-1, Rab7 or Rab5.

Various techniques known in the art are applicable for validation and characterization of a vesicle. Techniques useful for validation and characterization of a vesicle includes, but is not limited to, western blot, electron microscopy, immunohistochemistry, immunoelectron microscopy, FACS (Fluorescent activated cell sorting), electrophoresis (1 dimension, 2 dimension), liquid chromatography, mass spectrometry, MALDI-TOF (matrix assisted laser desorption/ionization-time of flight), ELISA, LC-MS-MS, and nESI (nanoelectrospray ionization). For example U.S. Pat. No. 2009/0148460 describes use of an ELISA method to characterize a vesicle. U.S. Pat. No. 2009/0258379 describes isolation of membrane vesicles from biological fluids.

Vesicles can be further analyzed for one or more nucleic acids, lipids, proteins or polypeptides, such as surface proteins or peptides, or proteins or peptides within a vesicle. Candidate peptides can be identified by various techniques including mass spectrometry coupled with purification methods such as liquid chromatography. A peptide can then be isolated and its sequence can be identified by sequencing. A computer program that predicts a sequence based on exact mass of a peptide can also be used to reveal the sequence of a peptide isolated from a vesicle. For example, LTQ-Orbitrap mass spectrometry can be used for high sensitivity and high accuracy peptide sequencing. LTQ-Orbitrap method has been described (Simpson et al, Expert Rev. Proteomics 6:267-283, 2009), which is incorporated herein by reference in its entirety.

### Vesicle Compositions

Also provided herein is an isolated vesicle with a particular biosignature. The isolated vesicle can comprise one or more biomarkers or biosignatures specific for specific cell type, or for characterizing a phenotype, such as described above. An isolated vesicle can also comprise one or more biomarkers, wherein the expression level of the one or more biomarkers is higher, lower, or the same for an isolated vesicle as compared to an isolated vesicle derived from a normal cell (ie. a cell derived from a subject without a phenotype of interest). For example, an isolated vesicle can comprise one or more biomarkers selected from Table 5. In an embodiment, the one or more biomarkers are selected from the group consisting of: B7H3, PSCA, MFG-E8, Rab, STEAP, PSMA, PCSA, 5T4, miR-9, miR-629, miR-141, miR-671-3p, miR-491, miR-182, miR-125a-3p, miR-324-5p, miR-148b, and miR-222, wherein the expression level of the one or more biomarkers is higher for an isolated vesicle as compared those derived from a normal cell. The biomarkers can comprise one or more of ADAM-10, BCNP, CD9, EGFR, EpCam, IL1B, KLK2, MMP7, p53, PBP, PCSA, SERPINB3, SPDEF, SSX2 and SSX4. For example, the biomarkers can be one or more of EGFR, EpCAM, KLK2, PBP, SPDEF, SSX2 and SSX4. The isolated vesicle can comprise at least 2, 3, 4, 5, 6, 7, 8, 9, 10, 11, 13, 14, 15, 16, 17, 18, or 19 of the biomarkers selected from the group. The isolated vesicle can further comprising one or more biomarkers selected from the group consisting of: EpCam, B7H3, PSMA, PSCA, PCSA, CD63, CD59, CD81, or CD9. The isolated vesicles can be PCSA+, Muc2+, Adam10+ vesicles. The isolated vesicles can be MMP7+ vesicles. The isolated vesicles can be Ago+ vesicles.

A composition comprising an isolated vesicle is also provided herein. The composition can comprise one or more isolated vesicles. For example, the composition can comprise a plurality of vesicles, or one or more populations of vesicles. The composition can be substantially enriched for vesicles. For example, the composition can be substantially absent of cellular debris, cells, or non-exosomal proteins, peptides, or nucleic acids (such as biological molecules not contained within the vesicles). The cellular debris, cells, or non-exosomal proteins, peptides, or nucleic acids, can be present in a biological sample along with vesicles. A composition can be substantially absent of cellular debris, cells, or non-exosomal proteins, peptides, or nucleic acids (such as biological molecules not contained within the vesicles), can be obtained by any method disclosed herein, such as through the use of one or more binding agents or capture agents for one or more vesicles. The vesicles can comprise at least 30, 40, 50, 60, 70, 80, 90, 95 or 99% of the total composition, by weight or by mass. The vesicles of the composition can be a heterogeneous or homogeneous population of vesicles. For example, a homogeneous population of vesicles comprises vesicles that are homogeneous as to one or more properties or characteristics. For example, the one or more characteristics can be selected from a group consisting of: one or more of the same biomarkers, a substantially similar or identical biosignature, derived from the same cell type, vesicles of a particular size, and a combination thereof.

Thus, in some embodiments, the composition comprises a substantially enriched population of vesicles. The composition can be enriched for a population of vesicles that are at least 30, 40, 50, 60, 70, 80, 90, 95 or 99% homogeneous as to one or more properties or characteristics. For example, the one or more characteristics can be selected from a group consisting of: one or more of the same biomarkers, a substantially similar or identical biosignature, derived from the same cell type, vesicles of a particular size, and a combination thereof. For example, the population of vesicles can be homogeneous by all having a particular biosignature, having the same biomarker, having the same biomarker combination, or derived from the same cell type. In some embodients, the composition comprises a substantially homogeneous population of vesicles, such as a population with a specific biosignature, derived from a specific cell, or both.

The population of vesicles can comprise one or more of the same biomarkers. The biomarker can be any component such as any nucleic acid (e.g. RNA or DNA), protein, peptide, polypeptide, antigen, lipid, carbohydrate, or proteoglycan. For example, each vesicle in a population can comprise the same or identical one or more biomarkers. In some embodiments, each vesicle comprises the same 1, 2, 3, 4, 5, 6, 3, 4, 5, 6, 7, 8, 9, 10, 11, 12, 13, 14, 15, 16, 17, 18, 19, 20, 25, 50, 75 or 100 biomarkers.

The vesicle population comprising the same or identical biomarker can include each vesicle in the population having the same presence or absence, expression level, mutational state, or modification of the biomarker. For example, an enriched population of vesicle can comprise vesicles wherein each vesicle has the same biomarker present, the same biomarker absent, the same expression level of a biomarker, the same modification of a biomarker, or the same mutation of a biomarker. The same expression level of a biomarker can refer to a quantitative or qualitive measurement, such as the vesicles in the population underexpress, overexpress, or have the same expression level of a biomarker as compared to a reference level.

Alternatively, the same expression level of a biomarker can be a numerical value representing the expression of a biomarker that is similar for each vesicle in a population. For example the copy number of a miRNA, the amount of protein, or the level of mRNA of each vesicle, can be quantitatively similar for each vesicle in a population, such that the numerical amount of each vesicle is ±1, 2, 3, 4, 5, 6, 7, 8, 9, 10, 15, or 20% from the amount in each other vesicle in the population, as such variations are appropriate.

In some embodiments, the composition comprises a substantially enriched population of vesicles, wherein the vesicles in the enriched population has a substantially similar or identical biosignature. The biosignature can comprise one or more characteristic of the vesicle, such as the level or amount of vesicles, temporal evaluation of the variation in vesicle half-life, circulating vesicle half-life, metabolic half-life of a vesicle, or the activity of a vesicle. The biosignature can also comprise the presence or absence, expression level, mutational state, or modification of a biomarker, such as those described herein.

The biosignature of each vesicle in the population can be at least 30, 40, 50, 60, 70, 80, 90, 95, or 99% identical. In some embodiments, the biosignature of each vesicle is 100% identical. The biosignature of each vesicle in the enriched population can have the same 1, 2, 3, 4, 5, 6, 3, 4, 5, 6, 7, 8, 9, 10, 11, 12, 13, 14, 15, 16, 17, 18, 19, 20, 25, 50, 75 or 100 characteristics. For example, a biosignature of a vesicle in an enriched population can be the presence of a first biomarker, the presence of a second biomarker, and the underexpression of a third biomarker. Another vesicle in the same population can be 100% identical, having the same first and second biomarkers present and underexpression of the third biomarker. Alternatively, a vesicle in the same population can have the same first and second biomarkers, but not have underexpression of the third biomarker.

In some embodiments, the composition comprises a substantially enriched population of vesicles, wherein the vesicles are derived from the same cell type. For example, the vesicles can all be derived from cells of a specific tissue, cells from a specific tumor of interest or a diseased tissue of interest, circulating tumor cells, or cells of maternal or fetal origin. The vesicles can all be derived from tumor cells. The vesicles can all be derived from the same tissue or cells, including without limitation lung, pancreas, stomach, intestine, bladder, kidney, ovary, testis, skin, colorectal, breast, prostate, brain, esophagus, liver, placenta, or fetal cells.

The composition comprising a substantially enriched population of vesicles can also comprise vesicles are of a particular size. For example, the vesicles can all a diameter of greater than about 10, 20, or 30 nm. They can all have a diameter of about 10-1000 nm, e.g., about 30-800 nm, about 30-200 nm, or about 30-100 nm. In some embodiments, the vesicles can all have a diameter of less than 10,000 nm, 1000 nm, 800 nm, 500 nm, 200 nm, 100 nm or 50 nm.

The population of vesicles homogeneous for one or more characteristics can comprises at least about 30, 40, 50, 60, 70, 80, 90, 95, or 99% of the total vesicle population of the composition. In some embodiments, a composition comprising a substantially enriched population of vesicles comprises at least 2, 3, 4, 5, 10, 20, 25, 50, 100, 250, 500, or 1000 times the concentration of vesicle as compared to a concentration of the vesicle in a biological sample from which the composition was derived. In yet other embodiments, the composition can further comprise a second enriched population of vesicles, wherein the poplulation of vesicles is at least 30% homogeneous as to one or more characteristics, as described herein.

Multiplex analysis can be used to obtain a composition substantially enriched for more than one population of vesicles, such as at least 2, 3, 4, 5, 6, 7, 8, 9, 10 vesicle, populations. Each substantially enriched vesicle population can comprise at least 5, 10, 15, 20, 25, 30, 35, 40, 45, 46, 47, 48, or 49% of the composition, by weight or by mass. In some embodiments, the substantially enriched vesicle population comprises at least about 30, 40, 50, 60, 70, 80, 90, 95, or 99% of the composition, by weight or by mass.

A substantially enriched population of vesicles can be obtained by using one or more methods, processes, or systems as disclosed herein. For example, isolation of a population of vesicles from a sample can be performed by using one or more binding agents for one or more biomarkers of a vesicle, such as using two or more binding agents that target two or more biomarkers of a vesicle. One or more capture agents can be used to obtain a substantially enriched population of vesicles. One or more detection agents can be used to identify a substantially enriched population of vesicles.

In one embodiment, a population of vesicles with a particular biosignature is obtained by using one or more binding agents for the biomarkers of the biosignature. The vesicles can be isolated resulting in a composition comprising a substantially enriched population of vesicles with the particular biosignature. In another embodiment, a population of vesicles with a particular biosignature of interest can be obtained by using one or more binding agents for biomarkers that are not a component of the biosignature of interest. Thus, the binding agents can be used to remove the vesicles that do not have the biosignature of interest and the resulting composition is substantially enriched for the population of vesicles with the particular biosignature of interest. The resulting composition can be substantially absent of the vesicles comprising a biomarker for the binding agent.

International Patent Application Serial No. PCT/US2011/031479, entitled “Circulating Biomarkers for Disease” and filed Apr. 6, 2011, which application is incorporated by reference in its entirety herein.

Mutation Associated Theranostics

Mutational or sequence analysis can be performed using any number of techniques described herein or known in the art, including without limitation sequencing (e.g., Sanger, Next Generation, pyrosequencing), PCR, variants of PCR such as RT-PCR, fragment analysis, and the like. Table 12 describes a number of genes bearing mutations that have been identified in various cancer lineages. In an aspect, the invention provides a theranostic method comprising isolating a microvesicle population using methods as described herein, isolating nucleic acids from the isolated microvesicle population (i.e., the nucleic acids comprise microvesicle payload), and determining a sequence of a nucleic acid that may affect a drug efficacy. The microvesicle population may comprise all microvesicles isolated from a biological sample, e.g., using filtration or centrifugation methods to isolate microvesicles from a tissue sample or bodily fluid such as blood. The microvesicle population may also comprise a subpopulation, e.g., isolated using a binding agent to one or more surface antigen. These techniques can be combined as desired. Such methodology and useful surface antigens are described in further detail herein. The nucleic acids can be mRNAs. In one embodiment, the nucleic acid sequences are assessed using Next Generation sequencing methods, e.g., using a HiSeq/TruSeq system offered by Illumina Corporation (Austin, Tex.) or an Ion Torrent system from Life Technologies (Carlsbad, Calif.). In another embodiment, the nucleic acid sequences are assessed using pyrosequencing. One of skill will appreciate that the profiling may be used to identify candidate treatments for cancer lineages other than those described in Table 12. Clinical trials in the table can be found at www.clinicaltrials.gov using the indicated identifiers.

TABLE 12
Exemplary Mutated Genes and Gene Products and Related Therapies

| Biomarker | Description |
|---|---|
| ABL1 | Most CML patients have a chromosomal abnormality due to a fusion between Abelson |
| | (Abl) tyrosine kinase gene at chromosome 9 and break point cluster (Bcr) gene at |
| | chromosome 22 resulting in constitutive activation of the Bcr-Abl fusion gene. Imatinib is a |
| | Bcr-Abl tyrosine kinase inhibitor commonly used in treating CML patients. Mutations in |
| | the ABL1 gene are common in imatinib resistant CML patients which occur in 30-90% of |
| | the patients. However, more than 50 different point mutations in the ABL1 kinase domain |
| | may be inhibited by the second generation kinase inhibitors, dasatinib, bosutinib and |
| | nilotinib. The gatekeeper mutation, T315I that causes resistance to all currently approved |
| | TKIs accounts for about 15% of the mutations found in patients with imatinib resistance. |
| | BCR-ABL1 mutation analysis is recommended to help facilitate selection of appropriate |
| | therapy for patients with CML after treatment with imatinib fails. Agents that target this |
| | biomarker are in clinical trials, e.g.: NCT01528085. |
| STK11 | STK11, also known as LKB1, is a serine/threonine kinase. It is thought to be a tumor suppressor |
| | gene which acts by interacting with p53 and CDC42. It modulates the activity of AMP-activated |
| | protein kinase, causes inhibition of mTOR, regulates cell polarity, inhibits the cell cycle, and |
| | activates p53. Somatic mutations in STK11 are associated with a history of smoking and KRAS |
| | mutation in NSCLC patients. The frequency of STK11 mutation in lung adenocarcinomas |
| | ranges from 7%-30%. STK11 loss may play a role in development of metastatic disease in lung |
| | cancer patients. Mutations of this gene also drive progression of HPV-induced dysplasia to |
| | invasive, cervical cancer and hence STK11 status may be exploited clinically to predict the |
| | likelihood of disease recurrence. Agents that target STK11 are in clinical trials, e.g.: |
| | NCT01578551. |
| | In addition, germline mutations in STK11 are associated with Peutz-Jeghers syndrome |
| | which is characterized by early onset hamartomatous gastro-intestinal polyps and increased |
| | risk of breast, colon, gastric and ovarian cancer. |
| FGFR2 | FGFR2 is a receptor for fibroblast growth factor. Activation of FGFR2 through mutation |
| | and amplification has been noted in a number of cancers. Somatic mutations of the FGFR2 |
| | tyrosine kinase have been observed in endometrial carcinoma, lung squamous cell |
| | carcinoma, cervical carcinoma, and melanoma. In the endometrioid histology of |
| | endometrial cancer, the frequency of FGFR2 mutation is 16% and the mutation is |
| | associated with shorter disease free survival in patients diagnosed with early stage disease. |
| | Loss of function FGFR2 mutations occur in about 8% melanomas and contribute to |
| | melanoma pathogenesis. Functional polymorphisms in the FGFR2 promoter are associated |
| | with breast cancer susceptibility. Agents that target FGFR2 are in clinical trials, e.g.: |
| | NCT01379534. |
| | In addition, germline mutations in FGFR2 are associated with numerous medical conditions |
| | that include congenital craniofacial malformation disorders, Apert syndrome and the related |
| | Pfeiffer and Crouzon syndromes. |
| ERBB4 | ERBB4 is a member of the Erbb receptor family known to play a pivotal role in cell-cell |
| | signaling and signal transduction regulating cell growth and development. The most |
| | commonly affected signaling pathways are the PI3K-Akt and MAP kinase pathways. Erbb4 |
| | was found to be somatically mutated in 19% of melanomas and Erbb4 mutations may |
| | confer “oncogene addiction” on melanoma cells. Erbb4 mutations have also been observed |
| | in various other cancer types, including, gastric carcinomas (1.7%), colorectal carcinomas |
| | (0.68-2.9%), non-small cell lung cancer (2.3-4.7%) and breast carcinomas (1.1%), |
| | however, their biological impact is not uniform or consistent across these cancers. Agents |
| | that target ERBB4 are in clinical trials, e.g.: NCT0126408. |
| SMARCB1 | SMARCB1 also known as SWI/SNF related, matrix associated, actin dependent regulator |
| | of chromatin, subfamily b, member 1, is a tumor suppressor gene implicated in cell growth |
| | and development. Loss of expression of SMARCB1 has been observed in tumors including |
| | epithelioid sarcoma, renal medullary carcinoma, undifferentiated pediatric sarcomas, and a |
| | subset of hepatoblastomas. |
| | In addition, germline mutation in SMARCB1 causes about 20% of all rhabdoid tumors |
| | which makes it important for clinicians to facilitate genetic testing and refer families for |
| | genetic counseling. Germline SMARCB1 mutations have also been identified as the |
| | pathogenic cause of a subset of schwannomas and meningiomas. |
| CDKN2A | CDKN2A or cyclin-dependent kinase inhibitor 2A is a tumor suppressor gene that encodes |
| | two cell cycle regulatory proteins p16INK4A and p14ARF. As upstream regulators of the |
| | retinoblastoma (RB) and p53 signaling pathways, CDKN2A controls the induction of cell |
| | cycle arrest in damaged cells that allows for repair of DNA. Loss of CDKN2A through |
| | whole-gene deletion, point mutation, or promoter methylation leads to disruption of these |
| | regulatory proteins and consequently dysregulation of growth control. Somatic CDKN2A |
| | mutations are documented to occur in squamous cell lung cancers, head and neck cancer, |
| | colorectal cancer, chronic myelogenous leukemia and malignant pleural mesothelioma. |
| | Currently, there are agents that target downstream of CDKN2A such as CDK4/6 inhibitors |
| | which function by restoring the cell's ability to induce cell cycle arrest. CDK4/6 inhibitors |
| | are in clinical trials for advanced solid tumors, including LEE011 (NCT01237236) and |
| | PD0332991 (NCT01522989, NCT01536743, NCT01037790). |
| | In addition, germline CDKN2A mutations are associated with melanoma-pancreatic |
| | carcinoma syndrome, which increases the risk for familial malignant melanoma and |
| | pancreatic cancer. |
| CTNNB1 | CTNNB1 or cadherin-associated protein, beta 1, encodes for β-catenin, a central mediator |
| | of the Wnt signaling pathway which regulates cell growth, migration, differentiation and |
| | apoptosis. Mutations in CTNNB1 (often occurring in exon 3) avert the breakdown of β- |
| | catenin, which allows the protein to accumulate resulting in persistent transactivation of |
| | target genes including c-myc and cyclin-D1. Somatic CTNNB1 mutations account for 1-4% |
| | of colorectal cancers, 2-3% of melanomas, 25-38% of endometrioid ovarian cancers, |
| | 84-87% of sporadic desmoid tumors, as well as the pediatric cancers, hepatoblastoma, |
| | medulloblastoma and Wilms' tumors. Compounds that suppress the Wnt/β-catenin pathway |
| | are available in clinical trials including PRI-724 for advanced solid tumors (NCT01302405) |
| | and LGK974 for melanoma and lobular breast cancer. |
| FGFR1 | FGFR1, or fibroblast growth factor receptor 1, encodes for FGFR1 which is important for |
| | cell division, regulation of cell maturation, formation of blood vessels, wound healing and |
| | embryonic development. Somatic activating mutations have been documented in |
| | melanoma, glioblastoma, and lung tumors. Other aberrations of FGFR1 including protein |
| | overexpression and gene amplification are common in breast cancer, squamous cell lung |
| | cancer, colorectal cancer, and, to some extent in adenocarcinoma of the lung. Recently, it |
| | has been shown that osteosarcoma and advanced solid tumors that exhibit FGFR1 |
| | amplification are sensitive to the pan-FGFR inhibitor, NVP-BGJ398. Other FGFR1- |
| | targeted agents under clinical investigation include dovitinib (NCT01440959). |
| | In addition, germline, gain-of-function mutations in FGFR1 result in developmental |
| | disorders including Kallmann syndrome and Pfeiffer syndrome. |
| FLT3 | FLT3, or Fms-like tyrosine kinase 3 receptor, is a member of class III receptor tyrosine |
| | kinase family, which includes PDGFRA/B and KIT. Signaling through FLT3 ligand- |
| | receptor complex regulates hematopoiesis, specifically lymphocyte development. The |
| | FLT3 internal tandem duplication (FLT3-ITD) is the most common genetic lesion in acute |
| | myeloid leukemia (AML), occurring in 25% of cases. FLT3 mutations are as common in |
| | solid tumors but have been documented in breast cancer. Several small molecule |
| | multikinase inhibitors targeting the RTK-III family are in clinical trials, including phase II |
| | trials for crenolanib in AML (NCT01657682), famitinib for nasopharyngeal carcinoma |
| | (NCT01462474), dovitinib for GIST (NCT01440959), and phase I trial for PLX108-01 in |
| | solid tumors (NCT01004861). |
| NOTCH1 | NOTCH1, or notch homolog 1, translocation-associated, encodes a member of the Notch |
| | signaling network, an evolutionary conserved pathway that regulates developmental |
| | processes by regulating interactions between physically adjacent cells. Notch signaling |
| | modulates interplay between tumor cells, stromal matrix, endothelial cells and immune |
| | cells, and mutations in NOTCH1 play a central role in disruption of microenvironmental |
| | communication, potentially leading to cancer progression. Due to the dual, bi-directional |
| | signaling of NOTCH1, activating mutations have been found in ALL and CLL, however |
| | loss of function mutations in NOTCH1 are prevalent in 11-15% of HNSCC. NOTCH1 |
| | mutations have also been found in 2% of glioblastomas, ~1% of ovarian cancers, 10% lung |
| | adenocarcinomas, 8% of squamous cell lung cancers and 5% of breast cancers. Notch |
| | pathway-directed therapy approaches differ depending on whether the tumor harbors gain |
| | or loss of function mutations, thus are classified as Notch pathway inhibitors or activators, |
| | respectively. Notch pathway modulators are being investigated in clinical trials, including |
| | MK0752 for advanced solid tumors (NCT01295632) and panobinostat (LBH589) for |
| | various refractory hematologic malignancies and many types of solid tumors including |
| | thyroid cancer (NCT01013597) and melanoma (NCT01065467). |
| NPM1 | NPM1, or nucleophosmin, is a nucleolar phosphoprotein belonging to a family of nuclear |
| | chaperones with proliferative and growth-suppressive roles. In several hematological |
| | malignancies, the NPM locus is lost or translocated, leading to expression of oncogenic |
| | proteins. NPM1 is mutated in one-third of patients with adult AML and leads to aberrant |
| | localization in the cytoplasm leading to activation of downstream pathways including |
| | JAK/STAT, RAS/ERK, and PI3K, leading to cell proliferation, survival and cytoskeletal |
| | rearrangements. In addition, the most common translocation in anaplastic large cell |
| | lymphoma (ALCL) is the NPM-ALK translocation which leads to expression of an |
| | oncogenic fusion protein with constitutive kinase activity. AML cells with mutant NPM are |
| | more sensitive to some chemotherapeutic agents including daunorubicin and camptothecin. |
| | ALK-targeted therapies such as crizotinib are under clinical investigation for ALK-NPM |
| | positive ALCL (NCT00939770). |
| SRC | SRC, or c-Src is a non-receptor tyrosine kinase, plays a critical role in cellular growth, |
| | proliferation, adhesion and angiogenesis. Normally maintained in a repressed state by |
| | intramolecular interactions involving the SH2 and SH3 domains, Src mutation prevents |
| | these restrictive intramolecular interactions, conferring a constitutively active state. |
| | Mutations are found in 12% of colon cancers (especially those metastatic to the liver) and |
| | 1-2% of endometrial cancers. Agents that target SRC are in clinical trials, e.g.: dasatinib for |
| | treatment of GIST (NCT01643278), endometrial cancer (NCT01440998), and other solid |
| | tumors (NCT01445509); saracatinib (AZD0530) for breast (NCT01216176) and pancreatic |
| | (NCT00735917) cancers; and bosutinib (SKI-606) for glioblastoma (NCT01331291). |
| SMAD4 | SMAD4, or mothers against decapentaplegic homolog 4, is one of eight proteins in the |
| | SMAD family, whose members are involved in multiple signaling pathways and are key |
| | modulators of the transcriptional responses to the transforming growth factor-β (TGFβ) |
| | receptor kinase complex. SMAD4 resides on chromosome 18q21, one of the most |
| | frequently deleted chromosomal regions in colorectal cancer. Smad4 stabilizes Smad DNA- |
| | binding complexes and also recruits transcriptional coactivators such as histone |
| | acetyltransferases to regulatory elements. Dysregulation of SMAD4 may occur late in |
| | tumor development, and can occur through mutations of the MH1 domain which inhibits |
| | the DNA-binding function, thus dysregulating TGFβR signaling. Mutated (inactivated) |
| | SMAD4 is found in 50% of pancreatic cancers and 10-35% of colorectal cancers. Studies |
| | have shown that preservation of SMAD4 through retention of the 18q21 region, leads to |
| | clinical benefit from 5-fluorouracil-based therapy. In addition, various clinical trials |
| | investigating agents which target the TGFβR signaling axis are available including PF- |
| | 03446962 for advanced solid tumors including NCT00557856. |
| | In addition, germline mutations in SMAD4 are associated with juvenile polyposis (JP) and |
| | combined syndrome of JP and hereditary hemorrhagic teleangiectasia (JP-HHT). |
| FBXW7 | FBXW7, or E3 ligase F-box and WD repeat domain containing 7, also known as Cdc4, |
| | encodes three protein isoforms which constitute a component of the ubiquitin-proteasome |
| | complex. Mutation of FBXW7 occurs in hotspots and disrupts the recognition of and |
| | binding with substrates which inhibits the proper targeting of proteins for degradation (e.g. |
| | Cyclin E, c-Myc, SREBP1, c-Jun, Notch-1 and mTOR). Mutation frequencies identified in |
| | cholangiocarcinomas, T-ALL, and carcinomas of endometrium, colon and stomach are |
| | 35%, 31%, 9%, 9%, and 6%, respectively. Therapeutic strategies comprise targeting an |
| | oncoprotein downstream of FBXW7, such as mTOR or c-Myc. Tumor cells with mutated |
| | FBXW7 are particularly sensitive to rapamycin treatment, indicating FBXW7 loss |
| | (mutation) can be a predictive biomarker for treatment with inhibitors of the mTOR |
| | pathway. |
| PTEN | PTEN, or phosphatase and tensin homolog, is a tumor suppressor gene that prevents cells |
| | from proliferating. PTEN is an important mediator in signaling downstream of EGFR, and |
| | loss of PTEN gene function/expression due to gene mutations or allele loss is associated |
| | with reduced benefit to EGFR-targeted monoclonal antibodies. Mutation in PTEN is found |
| | in 5-14% of colorectal cancer and 7% of breast cancer. PTEN mutation is generally related |
| | to loss of function of the encoded phosphatase, and an upregulation of the PIK3CA/AKT |
| | pathway. The role of PTEN loss in response to PIK3CA and mTOR inhibitors has been |
| | evaluated in some clinical studies. Agents that target PTEN and/or its downstream or |
| | upstream effectors are in clinical trials, including the following: NCT01430572, |
| | NCT01306045. |
| | In addition, germline PTEN mutations associate with Cowden disease and Bannayan-Riley- |
| | Ruvalcaba syndrome. These dominantly inherited disorders belong to a family of |
| | hamartomatous polyposis syndromes which feature multiple tumor-like growths |
| | (hamartomas) accompanied by an increased risk of breast carcinoma, follicular carcinoma |
| | of the thyroid, glioma, prostate and endometrial cancer. Trichilemmoma, a benign, |
| | multifocal neoplasm of the skin is also associated with PTEN germline mutations. |
| TP53 | TP53, or p53, plays a central role in modulating response to cellular stress through |
| | transcriptional regulation of genes involved in cell-cycle arrest, DNA repair, apoptosis, and |
| | senescence. Inactivation of the p53 pathway is essential for the formation of the majority of |
| | human tumors. Mutation in p53 (TP53) remains one of the most commonly described |
| | genetic events in human neoplasia, estimated to occur in 30-50% of all cancers with the |
| | highest mutation rates occurring in head and neck squamous cell carcinoma and colorectal |
| | cancer. Generally, presence of a disruptive p53 mutation is associated with a poor |
| | prognosis in all types of cancers, and diminished sensitivity to radiation and chemotherapy. |
| | Agents are in clinical trials which target p53's downstream or upstream effectors. Utility |
| | may depend on the p53 status. For p53 mutated patients, Chk1 inhibitors in advanced |
| | cancer (NCT01115790) and Wee1 inhibitors in refractory ovarian cancer (NCT01164995) |
| | are being investigated. For p53 wildtype patients with sarcoma, mdm2 inhibitors |
| | (NCT01605526) are being investigated. |
| | In addition, germline p53 mutations are associated with the Li-Fraumeni syndrome (LFS) |
| | which may lead to early-onset of several forms of cancer currently known to occur in the |
| | syndrome, including sarcomas of the bone and soft tissues, carcinomas of the breast and |
| | adrenal cortex (hereditary adrenocortical carcinoma), brain tumors and acute leukemias. |
| AKT1 | AKT1 gene (v-akt murine thymoma viral oncogene homologue 1) encodes a |
| | serine/threonine kinase which is a pivotal mediator of the PI3K-related signaling pathway, |
| | affecting cell survival, proliferation and invasion. Dysregulated AKT activity is a frequent |
| | genetic defect implicated in tumorigenesis and has been indicated to be detrimental to |
| | hematopoiesis. Activating mutation E17K has been described in breast (2-4%), endometrial |
| | (2-4%), bladder cancers (3%), NSCLC (1%), squamous cell carcinoma of the lung (5%) |
| | and ovarian cancer (2%). This mutation in the pleckstrin homology domain facilitates the |
| | recruitment of AKT to the plasma membrane and subsequent activation by altering |
| | phosphoinositide binding. A mosaic activating mutation E17K has also been suggested to |
| | be the cause of Proteus syndrome. Mutation E49K has been found in bladder cancer, which |
| | enhances AKT activation and shows transforming activity in cell lines. Agents targeting |
| | AKT1 are in clinical trials, e.g., the AKT inhibitor MK-2206 is in trials for patients |
| | carrying AKT mutations (see NCT01277757, NCT01425879). |
| ALK | APC, or adenomatous polyposis coli, is a key tumor suppressor gene that encodes for a |
| | large multi-domain protein. This protein exerts its tumor suppressor function in the Wnt/β- |
| | catenin cascade mainly by controlling the degradation of β-catenin, the central activator of |
| | transcription in the Wnt signaling pathway. The Wnt signaling pathway mediates important |
| | cellular functions including intercellular adhesion, stabilization of the cytoskeleton, and cell |
| | cycle regulation and apoptosis, and it is important in embryonic development and |
| | oncogenesis. Mutation in APC results in a truncated protein product with abnormal |
| | function, lacking the domains involved in β-catenin degradation. Somatic mutation in the |
| | APC gene can be detected in the majority of colorectal tumors (80%) and it is an early |
| | event in colorectal tumorigenesis. APC wild type patients have shown better disease control |
| | rate in the metastatic setting when treated with oxaliplatin, while when treated with |
| | fluoropyrimidine regimens, APC wild type patients experience more hematological |
| | toxicities. APC mutation has also been identified in oral squamous cell carcinoma, gastric |
| | cancer as well as hepatoblastoma and may contribute to cancer formation. Agents that |
| | target this gene and/or its downstream or upstream effectors are in clinical trials, e.g.: |
| | NCT01198743. |
| | In addition, germline mutation in APC causes familial adenomatous polyposis, which is an |
| | autosomal dominant inherited disease that will inevitably develop to colorectal cancer if |
| | left untreated. COX-2 inhibitors including celecoxib may reduce the recurrence of |
| | adenomas and incidence of advanced adenomas in individuals with an increased risk of |
| | CRC. Turcot syndrome and Gardner's syndrome have also been associated with germline |
| | APC defects. Germline mutations of the APC have also been associated with an increased |
| | risk of developing desmoid disease, papillary thyroid carcinoma and hepatoblastoma. |
| APC | APC, or adenomatous polyposis coli, is a key tumor suppressor gene that encodes for a |
| | large multi-domain protein. This protein exerts its tumor suppressor function in the Wnt/β- |
| | catenin cascade mainly by controlling the degradation of β-catenin, the central activator of |
| | transcription in the Wnt signaling pathway. Wnt signaling pathway mediates important |
| | cellular functions including intercellular adhesion, stabilization of the cytoskeleton and cell |
| | cycle regulation and apoptosis, and is important in embryonic development and |
| | oncogenesis. Mutation in APC results in a truncated protein product with abnormal |
| | function, lacking the domains involved in β-catenin degradation. Germline mutation is |
| | APC causes familial adenomatous polyposis, which is an autosomal dominant inherited |
| | disease that will inevitably develop to colorectal cancer if left untreated. Somatic mutation |
| | in APC gene can be detected in the majority of colorectal tumors (~80%) and is an early |
| | event in colorectal tumorigenesis. APC mutation has been identified in about 12.5% of oral |
| | squamous cell carcinoma and may contribute to the genesis of the cancer. |
| | Chemoprevention studies in preclinical models show APC deficient pre-malignant cells |
| | respond to a combination of TRAIL (tumor necrosis factor-related apoptosis-inducing |
| | ligand, or Apo2L) and RAc (9-cis-retinyl acetate) in vitro without normal cells being |
| | affected. |
| CDH1 | CDH1 (epithelial cadherin/E-cad) encodes a transmembrane calcium dependent cell |
| | adhesion glycoprotein that plays a major role in epithelial architecture, cell adhesion and |
| | cell invasion. Loss of function of CDH1 contributes to cancer progression by increasing |
| | proliferation, invasion, and/or metastasis. Various somatic mutations in CDH1 have been |
| | identified in diffuse gastric, lobular breast, endometrial and ovarian carcinomas; the |
| | resultant loss of function of E-cad can contribute to tumor growth and progression. |
| | In addition, germline mutations in CDH1 cause hereditary diffuse gastric cancer and |
| | colorectal cancer; affected women are predisposed to lobular breast cancer with a risk of |
| | about 50%. CDH1 mutation carriers have an estimated cumulative risk of gastric cancer of |
| | 67% for men and 83% for women, by age of 80 years. |
| C-Met | C-Met is a proto-oncogene that encodes the tyrosine kinase receptor of hepatocyte growth |
| | factor (HGF) or scatter factor (SF). c-Met mutation causes aberrant MET signaling in |
| | various cancer types including renal papillary, hepatocellular, head and neck squamous, |
| | gastric carcinomas and non-small cell lung cancer. Activating point mutations of MET |
| | kinase domain can cause cancer of various types, and may also decrease endocytosis and/or |
| | degradation of the receptor, resulting in enhanced tumor growth and metastasis. Mutations |
| | in the juxtamembrane domain (exon 14, 15) results in the constitutive activation and show |
| | enhanced tumorigenicity. c-MET inhibitors are in clinical trials for patients carrying MET |
| | mutations, e.g.: NCT01121575, NCT00813384. |
| | Germline mutations in c-MET have been associated with hereditary papillary renal cell |
| | carcinoma. |
| HRAS | HRAS (homologous to the oncogene of the Harvey rat sarcoma virus), together with KRAS |
| | and NRAS, belong to the superfamily of RAS GTPase. RAS protein activates RAS-MEK- |
| | ERK/MAPK kinase cascade and controls intracellular signaling pathways involved in |
| | fundamental cellular processes such as proliferation, differentiation, and apoptosis. Mutant |
| | Ras proteins are persistently GTP-bound and active, causing severe dysregulation of the |
| | effector signaling. HRAS mutations have been identified in cancers from the urinary tract |
| | (10%-40%), skin (6%) and thyroid (4%) and they account for 3% of all RAS mutations |
| | identified in cancer. RAS mutations (especially HRAS mutations) occur (5%) in cutaneous |
| | squamous cell carcinomas and keratoacanthomas that develop in patients treated with |
| | BRAF inhibitor vemurafenib, likely due to the paradoxical activation of the MAPK |
| | pathway. Agents that target HRAS and/or its downstream or upstream effectors are in |
| | clinical trials, e.g.: NCT01306045. |
| | In addition, germline mutation in HRAS has been associated with Costello syndrome, a |
| | genetic disorder that is characterized by delayed development and mental retardation and |
| | distinctive facial features and heart abnormalities. |
| IDH1 | IDH1 encodes for isocitrate dehydrogenase in cytoplasm and is found to be mutated in ~5% |
| | of primary gliomas and 60-90% of secondary gliomas, as well as in 12-18% of patients |
| | with acute myeloid leukemia. Mutated IDH1 results in impaired catalytic function of the |
| | enzyme, thus altering normal physiology of cellular respiration and metabolism. |
| | Furthermore, this mutation results in tumorigenesis. In gliomas, IDH1 mutations are |
| | associated with lower-grade astrocytomas and oligodendrogliomas (grade II/III). IDH gene |
| | mutations are associated with markedly better survival in patients diagnosed with malignant |
| | astrocytoma; and clinical data support a more aggressive surgery for IDH1 mutated patients |
| | because these individuals may be able to achieve long-term survival. In contrast, IDH1 |
| | mutation is associated with a worse prognosis in AML. In low-grade glioma patients |
| | receiving temozolomide before anaplastic transformation, IDH mutations (IDH1 and IDH2) |
| | have been shown to predict response to temozolomide. Agents that target IDH and/or its |
| | downstream or upstream effectors are in clinical trials, e.g.: NCT01534845. |
| JAK2 | JAK2 or Janus kinase 2 is a part of the JAK/STAT pathway which mediates multiple |
| | cellular responses to cytokines and growth factors including proliferation and cell survival. |
| | It is also essential for numerous developmental and homeostatic processes, including |
| | hematopoiesis and immune cell development. Mutations in the JAK2 kinase domain result |
| | in constitutive activation of the kinase and the development of chronic myeloproliferative |
| | neoplasms such as polycythemia vera (95%), essential thrombocythemia (50%) and |
| | myelofibrosis (50%). JAK2 mutations were also found in BCR-ABL1-negative acute |
| | lymphoblastic leukemia patients and the mutated patients show a poor outcome. Agents |
| | that target JAK2 and/or its downstream or upstream effectors are in clinical trials for |
| | patients carrying JAK2 mutations, e.g.: NCT00668421, NCT01038856. |
| | In addition, germline mutations in JAK2 have been associated with myeloproliferative |
| | neoplasms and thrombocythemia. |
| MPL | MPL or myeloproliferative leukemia gene encodes the thrombopoietin receptor, which is |
| | the main humoral regulator of thrombopoiesis in humans. MPL mutations cause |
| | constitutive activation of JAK-STAT signaling and have been detected in 5-7% of patients |
| | with primary myelofibrosis (PMF) and 1% of those with essential thrombocythemia (ET). |
| | In addition, germline mutations in MPL (S505N) have been associated with familial |
| | thrombocythemia. |
| PDGFRA | PDGFRA is the alpha subunit of platelet-derived growth factor receptor, a surface tyrosine |
| | kinase receptor, which can activate multiple signaling pathways including PIK3CA/AKT, |
| | RAS/MAPK and JAK/STAT. PDGFRA mutations are found in 5-8% of gastrointestinal |
| | stromal tumor cases, and in 40-50% of KIT wild type GISTs. Gain of function PDGFRA |
| | mutations confer imatinib sensitivity, while substitution mutation in exon 18 (D842V) |
| | shows resistance to the drug. A PDGFRA mutation in the extracellular domain was shown |
| | to identify a subgroup of DIPG (diffuse intrinsic pontine glioma) patients with significantly |
| | worse outcome PDGFRA inhibitors (e.g., crenolanib, pazopanib) are in clinical trials for |
| | patients carrying PDGFRA mutations, e.g.: NCT01243346, NCT01524848, NCT01478373. |
| | In addition, germline mutations in PDGFRA have been associated with Familial |
| | gastrointestinal stromal tumors and Hypereosinophillic Syndrome (HES). |
| SMO | SMO (smoothened) is a G protein-coupled receptor which plays an important role in the |
| | Hedgehog signaling pathway. It is a key regulator of cell growth and differentiation during |
| | development, and is important in epithelial and mesenchymal interaction in many tissues |
| | during embryogenesis. Dysregulation of the Hedgehog pathway is found in cancers |
| | including basal cell carcinomas (12%) and medulloblastoma (1%). A gain-of-function |
| | mutation in SMO results in constitutive activation of hedgehog pathway signaling, |
| | contributing to the genesis of basal cell carcinoma. SMO mutations have been associated |
| | with the resistance to SMO antagonist GDC-0449 in medulloblastoma patients. SMO |
| | mutation may also contribute to resistance to SMO antagonist LDE225 in BCC. SMO |
| | antagonists are in clinical trials, e.g.: NCT01529450. |
| VHL | VHL or von Hippel-Lindau gene encodes for tumor suppressor protein pVHL, which |
| | polyubiquitylates hypoxia-inducible factor in an oxygen dependent manner. Absence of |
| | pVHL causes stabilization of HIF and expression of its target genes, many of which are |
| | important in regulating angiogenesis, cell growth and cell survival. VHL somatic mutation |
| | has been seen in 20-70% of patients with sporadic clear cell renal cell carcinoma (ccRCC) |
| | and the mutation may imply a poor prognosis, adverse pathological features, and increased |
| | tumor grade or lymph-node involvement. Renal cell cancer patients with a ‘loss of |
| | function’ mutation in VHL show a higher response rate to therapy (bevacizumab or |
| | sorafenib) than is seen in patients with wild type VHL. Agents which target VHL and/or its |
| | downstream or upstream effectors are in clinical trials, e.g.: NCT01538238. |
| | In addition, germline mutations in VHL cause von Hippel-Lindau syndrome, associated |
| | with clear-cell renal-cell carcinomas, central nervous system hemangioblastomas, |
| | pheochromocytomas and pancreatic tumors. |
| ATM | ATM, or ataxia telangiectasia mutated, is activated by DNA double-strand breaks and DNA |
| | replication stress. It encodes a protein kinase that acts as a tumor suppressor and regulates |
| | various biomarkers involved in DNA repair, e.g., p53, BRCA1, CHK2, RAD17, RAD9, |
| | and NBS1. ATM is associated with hematologic malignancies, and somatic mutations have |
| | also been found in colon (18.2%), head and neck (14.3%), and prostate (11.9%) cancers. |
| | Inactivating ATM mutations may make patients more susceptible to PARP inhibitors. |
| | Agents that target ATM and/or its downstream or upstream effectors are in clinical trials, |
| | e.g.: NCT01311713. |
| | In addition, germline mutations in ATM are associated with ataxia-telangiectasia (also |
| | known as Louis-Bar syndrome) and a predisposition to malignancy. |
| CSF1R | CSF1R or colony stimulating factor 1 receptor gene encodes a transmembrane tyrosine |
| | kinase, a member of the CSF1/PDGF receptor family. CSF1R mediates the cytokine (CSF- |
| | 1) responsible for macrophage production, differentiation, and function. Mutations of this |
| | gene are associated with hematologic malignancies, as well as cancers of the liver (21.4%), |
| | colon (12.5%), prostate (3.3%), endometrium (2.4%), and ovary (2.4%). Patients with |
| | CSF1R mutations may respond to imatinib. Agents that target CSF1R and/or its |
| | downstream or upstream effectors are in clinical trials, e.g.: NCT01346358, NCT01440959. |
| | In addition, germline mutations in CSF1R are associated with diffuse leukoencephalopathy, |
| | a rapidly progressive neurodegenerative disorder. |
| FGFR3 | FGFR3 or fibroblast growth factor receptor type 3 gene encodes a member of the FGFR |
| | tyrosine kinase family, which include FGFR1, 2, 3, and 4. Dysregulation of FGFR3 has |
| | been implicated in activating the RAS-ERK pathway. FGFR3 has been found in various |
| | malignancies, including bladder cancer and multiple myeloma. Somatic mutations of this |
| | gene have also been found in skin (25.8%), head and neck (20.0%), and testicular (4.3%) |
| | cancers. Studies indicate FGFR3 and PIK3CA mutations occur together. FGFR3 mutations |
| | could serve as a strong prognostic indicator of a low recurrence rate in bladder cancer. |
| | Agents that target FGFR3 and/or its downstream or upstream effectors are in clinical trials, |
| | e.g.: NCT01004224. |
| | In addition, germline mutations in FGFR3 are associated with achondroplasia, |
| | hypochondroplasia, and Muenke syndrome, disorders involving but not limited to |
| | craniosynostosis and shortened extremities. FGFR3 is also associated with Crouzon |
| | syndrome with acanthosis nigricans. |
| GNAS | GNAS (or GNAS complex locus) encodes a stimulatory G protein alpha-subunit. These |
| | guanine nucleotide binding proteins (G proteins) are a family of heterotrimeric proteins |
| | which couple seven-transmembrane domain receptors to intracellular cascades. Stimulatory |
| | G-protein alpha-subunit transmits hormonal and growth factor signals to effector proteins |
| | and is involved in the activation of adenylate cyclases. Mutations of GNAS gene at codons |
| | 201 or 227 lead to constitutive cAMP signaling. GNAS somatic mutations have been found |
| | in pituitary (27.9%), pancreatic (19.2%), ovarian (11.4%), adrenal gland (6.2%), and colon |
| | (6.0%) cancers. SNPs in GNAS1 are a predictive marker for tumor response in |
| | cisplatin/fluorouracil-based radiochemotherapy in esophageal cancer. |
| | In addition, germline mutations of GNAS have been shown to be the cause of McCune- |
| | Albright syndrome (MAS), a disorder marked by endocrine, dermatologic, and bone |
| | abnormalities. GNAS is usually found as a mosaic mutation in patients. Loss of function |
| | mutations are associated with pseudohypoparathyroidism and |
| | pseudopseudohypoparathyroidism. |
| ERBB2 | ERBB2 (HER2) or v-erb-b2 erythroblastic leukemia viral oncogene homolog 2, |
| | neuro/glioblastoma derived oncogene homolog (avian) encodes a member of the epidermal |
| | growth factor (EGF) receptor family of receptor tyrosine kinases. This gene binds to other |
| | ligand-bound EGF receptor family members to form a heterodimer and enhances kinase- |
| | mediated activation of downstream signaling pathways, leading to cell proliferation. The |
| | most common mechanism for activation of HER2 is gene amplification, seen in |
| | approximately 15% of breast cancers. Somatic mutations have been found in colon (3.8%), |
| | endometrium (3.7%), prostate (3.0%), ovarian (2.5%), breast (1.7%) gastric (1.9%) cancers |
| | and 2-4% of lung adenocarcinomas. HER2 activated patients may respond to trastuzumab, |
| | afatinib, or lapatinib. Agents that target HER2 are in clinical trials, e.g.: NCT01306045. |
| HNF1A | HNF1A, or hepatocyte nuclear factor 1 homeobox A, encodes a transcription factor that is |
| | highly expressed in the liver, found on chromosome 12. It regulates a large number of |
| | genes, including those for albumin, alpha1-antitrypsin, and fibrinogen. HNF1A has been |
| | associated with an increased risk of pancreatic cancer. HNF1A somatic mutations are found |
| | in liver (30.1%), colon (14.5%), endometrium (11.1%), and ovarian (2.5%) cancers. |
| | In addition, germline mutations of HNF1A are associated with maturity-onset diabetes of |
| | the young type 3. |
| JAK3 | JAK3 or Janus activated kinase 3 is an intracellular tyrosine kinase involved in cytokine |
| | signaling, while interacting with members of the STAT family. Like JAK1, JAK2, and |
| | TYK2, JAK3 is a member of the JAK family of kinases. When activated, kinase enzymes |
| | phosphorylate one or more signal transducer and activator of transcription (STAT) factors, |
| | which translocate to the cell nucleus and regulate the expression of genes associated with |
| | survival and proliferation. JAK3 signaling is related to T cell development and |
| | proliferation. This biomarker is found in malignancies like head and neck (20.8%) colon |
| | (7.2%), prostate (4.8%), ovary (3.5%), breast (1.7%), lung (1.2%), and stomach (0.6%) |
| | cancer. |
| | In addition, germline mutations of JAK3 are associated with severe, combined |
| | immunodeficiency disease (SCID). |
| KDR | KDR (VEGFR2) or Kinase insert domain receptor gene, also known as vascular endothelial |
| | growth factor receptor-2 (VEGFR2), is involved with angiogenesis and is expressed on |
| | almost all endothelial cells. VEGF ligands bind to KDR, which leads to receptor |
| | dimerization and signal transduction. Somatic mutations in KDR have been observed in |
| | angiosarcoma (10.0%), and colon (12.7%), skin (12.7%), gastric (5.3%), lung (3.2%), renal |
| | (2.3%), and ovarian (1.9%) cancers. VEGFR antagonists that are FDA-approved or in |
| | clinical trials include bevacizumab, regorafenib, pazopanib, and vandetanib. Additional |
| | agents that target KDR and/or its downstream or upstream effectors are in clinical trials, |
| | e.g.: NCT01068587. |
| MLH1 | MLH1 or mutL homolog 1, colon cancer, nonpolyposis type 2 (E. coli) gene encodes a |
| | mismatch repair (MMR) protein which repairs DNA mismatches that occur during |
| | replication. Although the frequency is higher in colon cancer (10.4%), MLH1 somatic |
| | mutations have been found in esophageal (6.4%), ovarian (5.4%), urinary tract (5.3%), |
| | pancreatic (5.2%), and prostate (4.7%) cancers. Germline mutations of MLH1 are |
| | associated with Lynch syndrome, also known as hereditary non-polyposis colorectal cancer |
| | (HNPCC). Patients with Lynch syndrome are at increased risk for various malignancies, |
| | including intestinal, gynecologic, and upper urinary tract cancers and in its variant, Muir- |
| | Torre syndrome, with sebaceous tumors. |
| PTPN11 | PTPN11, or tyrosine-protein phosphatase non-receptor type 11, is a proto-oncogene that |
| | encodes a signaling molecule, Shp-2, which regulates various cell functions like mitogenic |
| | activation and transcription regulation. PTPN11 gain-of-function somatic mutations have |
| | been found to induce hyperactivation of the Akt and MAPK networks. Because of this |
| | hyperactivation, Ras effectors such as Mek and PI3K are targets for candidate therapies in |
| | those with PTPN11 gain-of-function mutations. PTPN11 somatic mutations are found in |
| | hematologic and lymphoid malignancies (8%), gastric (2.4%), colon (2%), ovarian (1.7%), |
| | and soft tissue (1.6%) cancers. |
| | In addition, germline mutations of PTPN11 are associated with Noonan syndrome, which |
| | itself is associated with juvenile myelomonocytic leukemia (JMML). PTPN11 is also |
| | associated with LEOPARD syndrome, which is associated with neuroblastoma and myeloid |
| | leukemia. |
| RB1 | RB1, or retinoblastoma-1, is a tumor suppressor gene whose protein regulates the cell cycle |
| | by interacting with various transcription factors, including the E2F family (which controls |
| | the expression of genes involved in the transition of cell cycle checkpoints). RB1 mutations |
| | have also been detected in ocular and other malignancies, such as ovarian (10.4%), bladder |
| | (41.3%), prostate (8.2%), breast (6.1%), brain (5.6%), colon (5.3%), and renal (1.5%) |
| | cancers. RB1 status, along with other mitotic checkpoints, has been associated with the |
| | prognosis of GIST patients. |
| | In addition, germline mutations of RB1 are associated with the pediatric tumor, |
| | retinoblastoma. Inherited retinoblastoma is usually bilateral. Patients with a history of |
| | retinoblastoma are at increased risk for secondary malignancies. |
| RET | RET or rearranged during transfection gene, located on chromosome 10, activates cell |
| | signaling pathways involved in proliferation and cell survival. RET mutations are mostly |
| | found in papillary thyroid cancers and medullary thyroid cancers (MTC), but RET fusions |
| | have also been found in 1% of lung adenocarcinomas. A 10-year study notes that medullary |
| | thyroid cancer patients with somatic mutations of RET correlate with a poor prognosis. |
| | Approximately 50% of patients with sporadic MTC have somatic RET mutations; 85% of |
| | these involve the M918T mutation, which is associated with a higher response rate to |
| | vandetanib in comparison to M918T negative patients. Agents that target RET are in |
| | clinical trials, e.g.: NCT00514046, NCT01582191. |
| | Germline activating mutations of RET are associated with multiple endocrine neoplasia |
| | type 2 (MEN2), which is characterized by the presence of medullary thyroid carcinoma, |
| | bilateral pheochromocytoma, and primary hyperparathyroidism. Germline inactivating |
| | mutations of RET are associated with Hirschsprung's disease. |
| c-Kit | c-Kit is a cytokine receptor expressed on the surface of hematopoietic stem cells as well as |
| | other cell types. This receptor binds to stem cell factor (SCF, a cell growth factor). As c-Kit |
| | is a receptor tyrosine kinase, ligand binding causes receptor dimerization and initiates a |
| | phosphorylation cascade resulting in changes in gene expression. These changes affect |
| | proliferation, apoptosis, chemotaxis and adhesion. C-KIT mutation has been identified in |
| | various cancer types including gastrointestinal stromal tumors (GIST) (up to 85%) and |
| | melanoma (7%). c-Kit is inhibited by multi-targeted agents including imatinib, sunitinib |
| | and sorafenib. Agents which target c-KIT and/or its downstream or upstream effectors are |
| | also in clinical trials for patients carrying c-KIT mutation, e.g.: NCT01028222, |
| | NCT01092728. |
| | In addition, germline mutations in c-KIT have been associated with multiple |
| | gastrointestinal stromal tumors (GIST) and Piebald trait. |
| EGFR | EGFR or epidermal growth factor receptor, is a transmembrane receptor tyrosine kinase |
| | belonging to the ErbB family of receptors. Upon ligand binding, the activated receptor |
| | triggers a series of intracellular pathways (Ras/MAPK, PI3K/Akt, JAK-STAT) that result |
| | in cell proliferation, migration and adhesion. Dysregulation of EGFR through mutation |
| | leads to ligand-independent activation and constitutive kinase activity, which results in |
| | uncontrolled growth and proliferation of many human cancers. EGFR mutations have been |
| | observed in 20-25% of non-small cell lung cancer (NSCLC), 10% of endometrial and |
| | peritoneal cancers. Somatic gain-of-function EGFR mutations, including in-frame deletions |
| | in exon 19 or point mutations in exon 21, confer sensitivity to first-generation EGFR- |
| | targeted tyrosine kinase inhibitors, whereas the secondary mutation, T790M in exon 20, |
| | confers resistance to tyrosine kinase inhibitors. New agents and combination therapies that |
| | include EGFR TKIs are in clinical trials for primary treatment of EGFR-mutated patients, |
| | including second-generation tyrosine kinase inhibitors such as icotinib (NCT01665417) for |
| | NSCLC or afatinib for advanced solid tumors (NCT00809133) and lung neoplasms |
| | (NCT01466660). In addition, new therapies and combination therapies are being explored |
| | for patients that have progressed on EGFR-targeted agents including afatinib |
| | (NCT01647711) for NSCLC. |
| | Germline mutations and polymorphisms of EGFR have been associated with familial lung |
| | adeocarcinomas. |
| PIK3CA | PIK3CA or phosphoinositide-3-kinase catalytic alpha polypeptide encodes a protein in the |
| | PI3 kinase pathway. This pathway is an active target for drug development. PIK3CA |
| | somatic mutations have been found in breast (26.1%), endometrial (23.3%), urinary tract |
| | (19.3%), colon (13.0%), and ovarian (10.8%) cancers. Somatic mosaic activating mutations |
| | in PIK3CA may cause CLOVES syndrome. PIK3CA mutations have been associated with |
| | benefit from mTOR inhibitors (e.g., everolimus, temsirolimus). Breast cancer patients with |
| | activation of the PI3K pathway due to PTEN loss or PIK3CA mutation/amplification may |
| | have a shorter survival following trastuzumab treatment. PIK3CA mutated (exon 20) |
| | colorectal cancer patients are less likely to respond to EGFR targeted monoclonal antibody |
| | therapy. Agents that target PIK3CA are in clinical trials, e.g.: NCT00877773, |
| | NCT01277757, NCT01219699, NCT01501604. |
| NRAS | NRAS is an oncogene and a member of the (GTPase) ras family, which includes KRAS |
| | and HRAS. This biomarker has been detected in multiple cancers including melanoma |
| | (15%), colorectal cancer (4%), AML (10%) and bladder cancer (2%). Acquired mutations |
| | in NRAS may be associated with resistance to vemurafenib in melanoma patients. In |
| | colorectal cancer patients NRAS mutation is associated with resistance to EGFR-targeted |
| | monoclonal antibodies. Agents which target this gene and/or its downstream or upstream |
| | effectors are in clinical trials, e.g.: NCT01306045, NCT01320085 |
| | In addition, germline mutations in NRAS have been associated with Noonan syndrome, |
| | autoimmune lymphoproliferative syndrome and juvenile myelomonocytic leukemia. |
| GNA11 | GNA11 is a proto-oncogene that belongs to the Gq family of the G alpha family of G |
| | protein coupled receptors. Known downstream signaling partners of GNA11 are |
| | phospholipase C beta and RhoA and activation of GNA11 induces MAPK activity. Over |
| | half of uveal melanoma patients lacking a mutation in GNAQ exhibit somatic mutations in |
| | GNA11. Agents that target GNA11 are in clinical trials, e.g.: NCT01587352, |
| | NCT01390818, NCT01143402. |
| GNAQ | GNAQ encodes the Gq alpha subunit of G proteins. G proteins are a family of |
| | heterotrimeric proteins coupling seven-transmembrane domain receptors. Oncogenic |
| | mutations in GNAQ result in a loss of intrinsic GTPase activity, resulting in a constitutively |
| | active Galpha subunit. This results in increased signaling through the MAPK pathway. |
| | Somatic mutations in GNAQ have been found in 50% of primary uveal melanoma patients |
| | and up to 28% of uveal melanoma metastases. Agents that target GNAQ are in clinical |
| | trials, e.g.: NCT01587352, NCT01390818, NCT01143402. |
| KRAS | KRAS, or V-Ki-ras2 Kirsten rat sarcoma viral oncogene homolog, encodes a signaling |
| | intermediate involved in many signaling cascades including the EGFR pathway. KRAS |
| | somatic mutations have been found in pancreatic (57.4%), colon (34.9%), lung (16.0%), |
| | biliary tract (28.2%), and endometrial (14.6%) cancers. Mutations at activating hotspots are |
| | associated with resistance to EGFR tyrosine kinase inhibitors (e.g., erlotinib, gefitinib) and |
| | monoclonal antibodies (e.g., cetuximab, panitumumab). Agents that target KRAS are in |
| | clinical trials, e.g.: NCT01248247, NCT01229150. |
| | In addition, germline mutations of KRAS (V14I, T58I, and D153V amino acid |
| | substitutions) are associated with Noonan syndrome. |
| BRAF | BRAF encodes a protein belonging to the raf/mil family of serine/threonine protein kinases. |
| | This protein plays a role in regulating the MAP kinase/ERK signaling pathway initiated by |
| | EGFR activation, which affects cell division, differentiation, and secretion. BRAF somatic |
| | mutations have been found in melanoma (43%), thyroid (39%), biliary tree (14%), colon |
| | (12%), and ovarian tumors (12%). Patients with mutated BRAF genes have a reduced |
| | likelihood of response to EGFR targeted monoclonal antibodies, such as cetuximab. A |
| | BRAF enzyme inhibitor, vemurafenib, was approved by FDA to treat unresectable or |
| | metastatic melanoma patients harboring BRAF V600E mutations. Agents that target BRAF |
| | are also in clinical trials, e.g.: NCT01543698, NCT01352273, NCT01709292. |
| | In addition, BRAF inherited mutations are associated with Noonan/Cardio-Facio- |
| | Cutaneous (CFC) syndrome, syndromes associated with short stature, distinct facial |
| | features, and potential heart/skeletal abnormalities. |

In an aspect, the invention provides a theranosis for a cancer which comprises mutational analysis of a panel of nucleic acids isolated from a microvesicle population, e.g., at least 2, 3, 4, 5, 6, 7, 8, 9, 10, 12, 15, 20, 25, 30, 35, 40, 45 or at least 50 genes. As described herein, the mutational analysis can be used to identify a candidate agent that is likely to benefit the cancer patient. The mutational analysis can also be used to identify a candidate agent that is not likely to benefit the cancer patient. A report can be generated that describes results of the mutational analysis. The report may include a summary of the mutational analysis for the genes assessed. The report may also provide a linkage of the mutational analysis with the predicted efficacy of various treatments based on the mutational analysis. The report may also comprise one or more clinical trials associated with one or more identified mutation in the patient.

The mutational analysis may be performed for one or more gene in Table 12. For example, the mutational analysis may be performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 genes in Table 12. In an embodiment, the mutational analysis is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of ABL1, AKT1, ALK, APC, ATM, BRAF, CDH1, CDKN2A, c-Kit, C-Met, CSF1R, CTNNB1, EGFR, ERBB2, ERBB4, FBXW7, FGFR1, FGFR2, FGFR3, FLT3, GNA11, GNAQ, GNAS, HNF1A, HRAS, IDH1, JAK2, JAK3, KDR, KRAS, MLH1, MPL, NOTCH1, NPM1, NRAS, PDGFRA, PIK3CA, PTEN, PTPN11, RB1, RET, SMAD4, SMARCB1, SMO, SRC, STK11, TP53, VHL. The mutational analysis may be performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAQ, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, the molecular profile may comprise mutational analysis of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53, and VHL. In an embodiment, the mutational analysis is performed in concert with other assessment of additional biomarkers provided herein. For example, the analysis of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, CSF1R, CTNNB1, EGFR, ERBB2 (HER2), ERBB4, FBXW7, FGFR1, FGFR2, FLT3, GNA11, GNAS, HNF1A, HRAS, IDH1, JAK2, JAK3, KDR (VEGFR2), KIT, KRAS, MET, MLH1, MPL, NOTCH1, NPM1, NRAS, PDGFRA, PIK3CA, PTEN, PTPN11, RB1, RET, SMAD4, SMARCB1, SMO, STK11, TP53 and VHL can be assessed in vesicles identified as expression one or more protein in Table 3, Table 4 or Table 5.

In another embodiment, the mutational analysis is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, VHL. For example, ABL1, AKT1, ALK, APC, ATM, BRAF, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCH1, NRAS, PDGFRA, PIK3CA, PTEN, RET, SMO, TP53, and VHL. As desired, additional biomarkers may be assessed for mutational analysis including at least 1, 2, 3, 4, 5, 6, 7, 8, 9, 10 or 11 of CDH1, ERBB4, FBXW7, HNF1A, JAK3, NPM1, PTPN11, RB1, SMAD4, SMARCB1, STK11. For example, CDH1, ERBB4, FBXW7, HNF1A, JAK3, NPM1, PTPN11, RB1, SMAD4, SMARCB1, STK11 may be assessed in addition to the biomarkers above. In an embodiment, the mutational analysis comprises that of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, VHL. For example, mutational analysis may comprise or consist of that of ABL1, AKT1, ALK, APC, ATM, BRAF, CDH1, cKIT, cMET, CSF1R, CTNNB1, EGFR, ERBB2, ERBB4, FBXW7, FGFR1, FGFR2, FLT3, GNA11, GNAQ, GNAS, HNF1A, HRAS, IDH1, JAK2, JAK3, KDR (VEGFR2), KRAS, MLH1, MPL, NOTCH1, NPM1, NRAS, PDGFRA, PIK3CA, PTEN, PTPN11, RB1, RET, SMAD4, SMARCB1, SMO, STK11, TP53, and VHL.

In still other embodiments, the mutational analysis may be performed for 1, 2, 3, 4, 5, 6, 7, 8, 9, 10, 11, 12, 13, 14, 15, 16, 17, 18, 19 or 20 of ALK, BRAF, BRCA1, BRCA2, EGFR, ERRB2, GNA11, GNAQ, IDH1, IDH2, KIT, KRAS, MET, NRAS, PDGFRA, PIK3CA, PTEN, RET, SRC, TP53. The mutational analysis may comprise that of 1, 2, 3, 4, 5, 6, 7, 8, 9, 10, 11, 12, 13, 14, 15, 16, 17, 18, 19, 20, 21, 22, 23, 24, 25, 26, 27 or 28 of AKT1, HRAS, GNAS, MEK1, MEK2, ERK1, ERK2, ERBB3, CDKN2A, PDGFRB, IFG1R, FGFR1, FGFR2, FGFR3, ERBB4, SMO, DDR2, GRB1, PTCH, SHH, PD1, UGT1A1, BIM, ESR1, MLL, AR, CDK4, SMAD4. The mutational analysis can be performed for 1, 2, 3, 4, 5, 6, 7, 8, 9, 10, 11, 12, 13, 14, 15, 16, 17, 18, 19, 20 or 21 of ABL, APC, ATM, CDH1, CSFR1, CTNNB1, FBXW7, FLT3, HNF1A, JAK2, JAK3, KDR, MLH1, MPL, NOTCH1, NPM1, PTPN11, RB1, SMARCB1, STK11, VHL. The genes assessed by mutational analysis may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, at least 200 genes, or all genes, selected from the group consisting of ABL1, AKT1, AKT2, AKT3, ALK, APC, AR, ARAF, ARFRP1, ARID1A, ARID2, ASXL1, ATM, ATR, ATRX, AURKA, AURKB, AXL, BAP1, BARD1, BCL2, BCL2L2, BCL6, BCOR, BCORL1, BLM, BRAF, BRCA1, BRCA2, BRIP1, BTK, CARD11, CBFB, CBL, CCND1, CCND2, CCND3, CCNE1, CD79A, CD79B, CDC73, CDH1, CDK12, CDK4, CDK6, CDK8, CDKN1B, CDKN2A, CDKN2B, CDKN2C, CEBPA, CHEK1, CHEK2, CIC, CREBBP, CRKL, CRLF2, CSF1R, CTCF, CTNNA1, CTNNB1, DAXX, DDR2, DNMT3A, DOT1L, EGFR, EMSY (C11orf30), EP300, EPHA3, EPHA5, EPHB1, ERBB2, ERBB3, ERBB4, ERG, ESR1, EZH2, FAM123B (WTX), FAM46C, FANCA, FANCC, FANCD2, FANCE, FANCF, FANCG, FANCL, FBXW7, FGF10, FGF14, FGF19, FGF23, FGF3, FGF4, FGF6, FGFR1, FGFR2, FGFR3, FGFR4, FLT1, FLT3, FLT4, FOXL2, GATA1, GATA2, GATA3, GID4 (C17orf39), GNA11, GNA13, GNAQ, GNAS, GPR124, GRIN2A, GSK3B, HGF, HRAS, IDH1, IDH2, IGF1R, IKBKE, IKZF1, IL7R, INHBA, IRF4, IRS2, JAKE JAK2, JAK3, JUN, KAT6A (MYST3), KDM5A, KDM5C, KDM6A, KDR, KEAP1, KIT, KLHL6, KRAS, LRP1B, MAP2K1, MAP2K2, MAP2K4, MAP3K1, MCL1, MDM2, MDM4, MED12, MEF2B, MEN1, MET, MITF, MLH1, MLL, MLL2, MPL, MRE11A, MSH2, MSH6, MTOR, MUTYH, MYC, MYCL1, MYCN, MYD88, NF1, NF2, NFE2L2, NFKBIA, NKX2-1, NOTCH1, NOTCH2, NPM1, NRAS, NTRK1, NTRK2, NTRK3, NUP93, PAK3, PALB2, PAX5, PBRM1, PDGFRA, PDGFRB, PDK1, PIK3CA, PIK3CG, PIK3R1, PIK3R2, PPP2R1A, PRDM1, PRKAR1A, PRKDC, PTCH1, PTEN, PTPN11, RAD50, RAD51, RAF1, RARA, RB1, RET, RICTOR, RNF43, RPTOR, RUNX1, SETD2, SF3B1, SMAD2, SMAD4, SMARCA4, SMARCB1, SMO, SOCS1, SOX10, SOX2, SPEN, SPOP, SRC, STAG2, STAT4, STK11, SUFU, TET2, TGFBR2, TNFAIP3, TNFRSF14, TOP1, TP53, TSC1, TSC2, TSHR, VHL, WISP3, WT1, XPO1, ZNF217, ZNF703. The mutational analysis may be performed to detect a gene rearrangement, e.g., a rearrangement in 1, 2, 3, 4, 5, 6, 7, 8, 9, 10, 11, 12, 13, 14, 15, 16, 17, 18 or 19 of ALK, BCR, BCL2, BRAF, EGFR, ETV1, ETV4, ETV5, ETV6, EWSR1, MLL, MYC, NTRK1, PDGFRA, RAF1, RARA, RET, ROS1, TMPRSS2.

In an embodiment, the mutational analysis is performed for the v-Ki-ras2 Kirsten rat sarcoma viral oncogene homolog (KRAS) gene. The KRAS gene encodes a protein that is a member of the small GTPase superfamily and is a signaling intermediate involved in various signaling cascades including the EGFR pathway. Once activated, KRAS recruits and activates proteins necessary for the propagation of growth factor and other receptor signals, such as c-Raf and PI 3-kinase.

A single amino acid substitution in KRAS from a single nucleotide substitution can be responsible for an activating mutation. The transforming protein that results is implicated in various malignancies, including lung adenocarcinoma, mucinous adenoma, ductal carcinoma of the pancreas and colorectal carcinoma. Somatic KRAS mutations are found at in various cancers, e.g., leukemias, colon cancer, pancreatic cancer and lung cancer. Mutations at activating hotspots are associated with resistance to EGFR tyrosine kinase inhibitors (erlotinib, gefitinib) and monoclonal antibodies (cetuximab, panitumumab).

In an aspect, the invention provides a method of determining a KRAS nucleotide sequence in a biological sample that comprises one or more microvesicle, comprising: (a) contacting the biological sample with a binding agent to a microvesicle surface antigen; (b) isolating nucleic acids from the microvesicles that formed a complex with the binding agent to the microvesicle surface antigen in step (a); and (c) determining a v-Ki-ras2 Kirsten rat sarcoma viral oncogene homolog (KRAS) sequence within the nucleic acids isolated in step (b). The microvesicle surface antigen can be selected to isolate a desired vesicle population. For example, a general vesicle marker may facilitate isolation of a majority of microvesicles in a sample and also differentiate microvesicles from other cellular debris or the like, a tissue specific marker may facilitate isolation of microvesicles in a sample from a given tissue or cell-specific origin, and a disease marker can facilitate isolation of microvesicles representative of a certain disease, e.g., a cancer. A population of microvesicles can be isolated using a plurality of surface antigens, e.g., to isolate microvesicles indicative of a cancer from a given cancer lineage. The surface antigen can be selected from Table 3, Table 4 or Table 5 herein. In an embodiment, the microvesicle surface antigen comprises Tissue factor, EpCam, B7H3, RAGE and/or CD24. The surface antigen may comprise CD24.

Multiple microvesicle surface antigens can be detected. For example, the method may further comprise contacting the biological sample with a binding agent to a general vesicle marker in step (a) and isolating the nucleic acids from microvesicles that also formed a complex with the binding agent to the general vesicle marker in step (b). In an embodiment, the general vesicle marker is selected from Table 3. The general vesicle marker can be a tetraspanin. The tetraspanin can be CD9, CD63 and/or CD81.

The KRAS sequence may be determined by pyrosequencing, chain-termination (e.g., dye-termination or Sanger sequencing), or Next Generation sequencing. The sequencing can be performed to determine whether the KRAS sequence comprises a mutation. The mutation can be an activating mutation. In an embodiment, the mutation comprises a 38G>A mutation in the nucleotide sequence. This mutation is also referred to as G13D. The G13D mutation results in an amino acid substitution at position 13 in KRAS, from a glycine (G) to an aspartic acid (D). Using similar terminology (i.e., nucleotide substitution (resulting amino acid substitution)), mutations in KRAS that may be detected include without limitation 1, 2, 3, 4, 5, 6, 7, 8, 9, 10, 11, 12, 13, 14, 15, 16, 17, 18, 19, 20, 21 or 22 of 34G>T (G12C), 34G>C (G12R), 34G>A (G12S), 35G>C (G12A), 35G>A (G12D), 35G>T (G12V), 37G>T (G13C), 37G>C (G13R), 37G>A (G13S), 38G>C (G13A), 38G>A (G13D), 38G>T (G13V), 181C>A (Q61K), 182A>T (Q61L), 182A>G (Q61R), 183A>C (Q61H), 183A>T (Q61H), 351A>C (K117N), 351A>T (K117N), 436G>C (A146P), 436G>A (A146T), and 437C>T (A146V).

The nucleic acids isolated in step (b) may comprise DNA or RNA, e.g., mRNA. In an embodiment, mRNAs are isolated from the microvesicle payload and an mRNA sequence is determined.

As described, the determined KRAS sequence may be used to provide a prognosis or a theranosis for a cancer. The theranosis comprises a therapy-related diagnosis or prognosis, e.g. the theranosis may comprise a prediction of whether a cancer is likely to respond or not respond to a chemotherapeutic agent. Accordingly, a treating physician or other caregiver can use such information to help determine whether to treat or not treat a patient with the chemotherapeutic agent.

In embodiments, the chemotherapeutic agent comprises an epidermal growth factor receptor (EGFR) directed therapy. The epidermal growth factor receptor (EGFR) is an important player in cancer initiation and progression. KRAS plays a role as an effector molecule responsible for signal transduction from ligand-bound EGFR to the nucleus. Tumors carrying KRAS mutations are unlikely to respond to EGFR-targeted monoclonal antibodies or experience survival benefit from such treatment. EGFR directed therapy includes without limitation panitumumab, cetuximab, zalutumumab, nimotuzumab, matuzumab, gefitinib, erlotinib, and/or lapatinib.

Mutations in KRAS may also affect the efficacy of treatments directed to other molecular targets. In embodiments, the chemotherapeutic agent comprises a mammalian target of rapamycin (mTOR) directed therapy, a mitogen-activated or extracellular signal-regulated protein kinase kinase (MEK) directed therapy, and/or a v-raf murine sarcoma viral oncogene homolog B1 (BRAF) directed therapy. Such mTOR directed therapies include without limitation everolimus and/or temsirolimus.

The chemotherapeutic agent may comprise a cyclophosphamide or a combination of vincristine+ carmustine (BCNU)+melphalan+cyclophosphamide+prednisone (VBMCP). These agents may be use to treat multiple myeloma (MM).

As described, a mutation in KRAS may be predictive that the cancer is less likely to respond to the chemotherapeutic agent. The cancer can be any appropriate cancer wherein KRAS may play a role in treatment selection. Accordingly, the cancer may include without limitation a solid tumor, a colorectal cancer (CRC), a pancreatic cancer, a non-small cell lung cancer (NSCLC), a bronchioloalveolar carcinoma (BAC) or adenocarcinoma (BAC subtype), a leukemia, or a multiple myeloma (MM).

The biological sample may comprise a cell culture, such that the microvesicles are derived from mitered cells. The biological sample may also comprise a sample from a subject, e.g., a solid tumor sample or a bodily fluid from the subject. Appropriate bodily fluids comprise without limation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, or a derivative of any thereof.

In some embodiments, the biological sample comprises peripheral blood or a derivative thereof, e.g., serum or plasma. In such embodiments, the method may comprise removal of one or more abundant protein, e.g., an abundant blood protein, from the biological sample prior to or during the isolation of the one or more microvesicle. For example, abundant proteins may be removed prior to contacting the microvesicle with the binding agent. Non-limiting examples one or more abundant protein that may be removed include one or more of albumin, IgG, transferrin, fibrinogen, fibrin, IgA, a2-Marcroglobulin, IgM, α1-Antitrypsin, complement C3, haptoglobulin, apolipoprotein A1, A3 and B; α1-Acid Glycoprotein, ceruloplasmin, complement C4, C1q, IgD, prealbumin (transthyretin), plasminogen, a derivative of any thereof, and a combination thereof. Further examples of abundant proteins that may be removed comprise Albumin, Immunoglobulins, Fibrinogen, Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin, Mannose-binding lectin (MBL), a derivative of any thereof, and a combination thereof.

Various methodologies can be used to deplete abundant proteins from the biological sample. In some embodiments, the one or more abundant protein is depleted by immunoaffinity, precipitation, or a combination thereof. Commercially available columns can be used such described herein. Depleting the one or more abundant protein may also comprise contacting the biological sample with thromboplastin to precipitate fibrinogen.

The binding agent used to form a complex with the microvesicle can comprise any useful reagent, including without limitation a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof. Preferable binding agents include without limitation antibodies and/or aptamers.

In an embodiment, the binding agent is tethered to a substrate. The binding agent may also comprise a label. When multiple binding agents are used, e.g., to identify microvesicles bearing a plurality of surface antigens, at least one binding agent can be tethered to a substrate and another binding agent can carry a label. This allows the label to identify microvesicles in complex with the tethered binding agent. In addition, multiple tethered binding agents can be used, e.g., in a series of columns, wells, or precipitations. Multiple labeled binding agents may be used as well. The Examples provide illustration of each of these applications.

As described herein, the one or more microvesicle may be subjected to size exclusion chromatography, density gradient centrifugation, differential centrifugation, nanomembrane ultrafiltration, immunoabsorbent capture, affinity purification, affinity capture, immunoassay, microfluidic separation, flow cytometry or combinations thereof. For example, a large microvesicle population can be isolated by size exclusion chromatography, density gradient centrifugation, differential centrifugation, and/or nanomembrane ultrafiltration, then a subpopulation can be further isolated using immunoabsorbent capture, affinity purification, affinity capture, immunoassay and/or flow cytometry. Microvesicles may be at least partially identified or isolated by size. In an embodiment, the one or more microvesicle has a diameter between 10 nm and 2000 nm. For example, the one or more microvesicle may have a diameter between 20 nm and 200 nm. In other embodiments, microvesicles with a size greater than 800 nm, e.g., >1000 nm, are interrogated.

Also as described herein, the method can include detecting one or more payload biomarker within the one or more microvesicle. For example, the one or more payload biomarker may comprise one or more nucleic acid, peptide, protein, lipid, antigen, carbohydrate, and/or proteoglycan. The nucleic acid may be DNA, mRNA, microRNA, snoRNA, snRNA, rRNA, tRNA, siRNA, hnRNA, or shRNA. In preferred embodiments, the one or more payload biomarker comprises microRNA and/or mRNA. The payload markers can be assessed as part of providing the theranosis.

### Detection System and Kits

Also provided is a detection system configured to determine one or more biosignatures for a vesicle. The detection system can be used to detect a heterogeneous population of vesicles or one or more homogeneous population of vesicles. The detection system can be configured to detect a plurality of vesicles, wherein at least a subset of the plurality of vesicles comprises a different biosignature from another subset of the plurality of vesicles. The detection system detect at least 2, 3, 4, 5, 6, 7, 8, 9, 10, 15, 20, 25, 30, 40, 50, 60, 70, 80, 90, or 100 different subsets of vesicles, wherein each subset of vesicles comprises a different biosignature. For example, a detection system, such as using one or more methods, processes, and compositions disclosed herein, can be used to detect at least 2, 3, 4, 5, 6, 7, 8, 9, 10, 15, 20, 25, 30, 40, 50, 60, 70, 80, 90, or 100 different populations of vesicles.

The detection system can be configured to assess at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different biomarkers for one or more vesicles. In some embodiments, the one or more biomarkers are selected from any of Tables 3-5, or as disclosed herein. The detection system can be configured to assess a specific population of vesicles, such as vesicles from a specific cell-of-origin, or to assess a plurality of specific populations of vesicles, wherein each population of vesicles has a specific biosignature.

The detection system can be a low density detection system or a high density detection system. For example, a low density detection system can detect up to 1, 2, 3, 4, 5, 6, 7, 8, 9, or 10 different vesicle populations, whereas a high density detection system can detect at least about 15, 20, 25, 50, or 100 different vesicle populations In another embodiment, a low density detection system can detect up to about 100, 200, 300, 400, or 500 different biomarkers, whereas a high density detection system can detect at least about 750, 1000, 2000, 3000, 4000, 5000, 6000, 7000, 8000, 9,000, 10,000, 15,000, 20,000, 25,000, 50,000, or 100,000 different biomarkers. In yet another embodiment, a low density detection system can detect up to about 100, 200, 300, 400, or 500 different biosignatures or biomarker combinations, whereas a high density detection system can detect at least about 750, 1000, 2000, 3000, 4000, 5000, 6000, 7000, 8000, 9,000, 10,000, 15,000, 20,000, 25,000, 50,000, or 100,000 biosignatures or biomarker combinations.

The detection system can comprise a probe that selectively hybridizes to a vesicle. The detection system can comprise a plurality of probes to detect a vesicle. In some embodiments, a plurality of probes is used to detect the amount of vesicles in a heterogeneous population of vesicles. In yet other embodiments, a plurality of probes is used to detect a homogeneous population of vesicles. A plurality of probes can be used to isolate or detect at least two different subsets of vesicles, wherein each subset of vesicles comprises a different biosignature.

A detection system, such as using one or more methods, processes, and compositions disclosed herein, can comprise a plurality of probes configured to detect, or isolate, such as using one or more methods, processes, and compositions disclosed herein at least 2, 3, 4, 5, 6, 7, 8, 9, 10, 15, 20, 25, 30, 40, 50, 60, 70, 80, 90, or 100 different subsets of vesicles, wherein each subset of vesicles comprises a different biosignature.

For example, a detection system can comprise a plurality of probes configured to detect at least 2, 3, 4, 5, 6, 7, 8, 9, 10, 15, 20, 25, 30, 40, 50, 60, 70, 80, 90, or 100 different populations of vesicles. The detection system can comprise a plurality of probes configured to selectively hybridize to at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different biomarkers for one or more vesicles. In some embodiments, the one or more biomarkers are selected from any of Tables 3-5, or as disclosed herein. The plurality of probes can be configured to assess a specific population of vesicles, such as vesicles from a specific cell-of-origin, or to assess a plurality of specific populations of vesicles, wherein each population of vesicles has a specific biosignature.

The detection system can be a low density detection system or a high density detection system comprising probes to detect vesicles. For example, a low density detection system can comprise probes to detect up to 1, 2, 3, 4, 5, 6, 7, 8, 9, or 10 different vesicle populations, whereas a high density detection system can comprise probes to detect at least about 15, 20, 25, 50, or 100 different vesicle populations. In another embodiment, a low density detection system can comprise probes to detect up to about 100, 200, 300, 400, or 500 different biomarkers, whereas a high density detection system can comprise probes to detect at least about 750, 1000, 2000, 3000, 4000, 5000, 6000, 7000, 8000, 9,000, 10,000, 15,000, 20,000, 25,000, 50,000, or 100,000 different biomarkers. In yet another embodiment, a low density detection system can comprise probes to detect up to about 100, 200, 300, 400, or 500 different biosignatures or biomarker combinations, whereas a high density detection system can comprise probes to detect at least about 750, 1000, 2000, 3000, 4000, 5000, 6000, 7000, 8000, 9,000, 10,000, 15,000, 20,000, 25,000, 50,000, or 100,000 biosignatures or biomarker combinations.

The probes can be specific for detecting a specific vesicle population, for example a vesicle with a particular biosignature, and as described above. A plurality of probes for detecting prostate specific vesicles is also provided. A plurality of probes can comprise probes for detecting one or more of the biomarkers in Tables 3-5. The plurality of probes can also comprise one or more probes for detecting one or more of the biomarkers in Tables 3-5.

A plurality of probes for detecting one or more miRNAs of a vesicle can comprise probes for detecting one or more of the following miRNAs: miR-9, miR-629, miR-141, miR-671-3p, miR-491, miR-182, miR-125a-3p, miR-324-5p, miR-148b, and miR-222. In another embodiment, the plurality of probes comprises one or more probes for detecting EpCam, CD9, PCSA, CD63, CD81, PSMA, B7H3, PSCA, ICAM, STEAP, and EGFR. In some embodiments, the plurality of probes comprises one or more probes for detecting EpCam, CD9, PCSA, CD63, CD81, PSMA, and B7H3. In other embodiments, the plurality of probes comprises one or more probes for detecting EpCam, CD9, PCSA, CD63, CD81, PSMA, B7H3, PSCA, ICAM, STEAP, and EGFR. In yet another embodiment, a subset of the plurality of probes are capture agents for one or more of EpCam, CD9, PCSA, CD63, CD81, PSMA, B7H3, PSCA, ICAM, STEAP, and EGFR, and another subset are probes for detecting one or more of CD9, CD63, and CD81. A plurality of probes can also comprises one or more probes for detecting r miR-92a-2*, miR-147, miR-574-5p, or a combination thereof. A plurality of probes can also comprise one or more probes for detecting miR-548c-5p, miR-362-3p, miR-422a, miR-597, miR-429, miR-200a, miR-200b or a combination thereof. A plurality of probes can also comprise one or more probes for detecting EpCam, CK, and CD45. In some embodiments, the one or more probes may be capture agents. In another embodiment, the probes may be detection agents. In yet another embodiment, the plurality of probes comprises capture and detection agents.

The probes, such as capture agents, may be attached to a solid substrate, such as an array or bead. Alternatively, the probes, such as detection agents, are not attached. The detection system may be an array based system, a sequencing system, a PCR-based system, or a bead-based system, such as described above. The detection system can also be a microfluidic device as described above.

The detection system may be part of a kit. Alternatively, the kit may comprise the one or more probe sets or plurality of probes, as described herein. The kit may comprise probes for detecting a vesicle or a plurality of vesicles, such as vesicles in a heterogeneous population. The kit may comprise probes for detecting a homogeneous population of vesicles. For example, the kit may comprise probes for detecting a population of specific cell-of-origin vesicles, or vesicles with the same specific biosignature.

In a related aspect, the invention provides a kit comprising one or more reagent to carry out the method of the invention. The one or more reagent can be selected from the group consisting of one or more binding agent specific for a microvesicle surface antigen, a chromatography column, filtration units, membranes, flow reagents, a buffer, equipment to remove a highly abundant protein, one or more population of microvesicles, and a combination thereof. The one or more reagent can be a capture agent and/or a detector agent such as described herein. The kit can contain instructions for performing one or more steps of the methods of the invention.

### Computer Systems

A vesicle can be assayed for molecular features, for example, by determining an amount, presence or absence of one or more biomarkers. The data generated can be used to produce a biosignature, which can be stored and analyzed by a computer system, such as shown in FIG. 3. The assaying or correlating of the biosignature with one or more phenotypes can also be performed by computer systems, such as by using computer executable logic.

A computer system, such as shown in FIG. 3, can be used to transmit data and results following analysis. Accordingly, FIG. 3 is a block diagram showing a representative example logic device through which results from a vesicle can be analyzed and the analysis reported or generated. FIG. 3 shows a computer system (or digital device) 800 to receive and store data generated from a vesicle, analyze of the data to generate one or more biosignatures, and produce a report of the one or more biosignatures or phenotype characterization. The computer system can also perform comparisons and analyses of biosignatures generated, and transmit the results. Alternatively, the computer system can receive raw data of vesicle analysis, such as through transmission of the data over a network, and perform the analysis.

The computer system 800 may be understood as a logical apparatus that can read instructions from media 811 and/or network port 805, which can optionally be connected to server 809 having fixed media 812. The system shown in FIG. 3 includes CPU 801, disk drives 803, optional input devices such as keyboard 815 and/or mouse 816 and optional monitor 807. Data communication can be achieved through the indicated communication medium to a server 809 at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party 822. The receiving party 822 can be but is not limited to an individual, a health care provider or a health care manager. Thus, the information and data on a test result can be produced anywhere in the world and transmitted to a different location. For example, when an assay is conducted in a differing building, city, state, country, continent or offshore, the information and data on a test result may be generated and cast in a transmittable form as described above. The test result in a transmittable form thus can be imported into the U.S. to receiving party 822. Accordingly, the present invention also encompasses a method for producing a transmittable form of information on the diagnosis of one or more samples from an individual. The method comprises the steps of (1) determining a diagnosis, prognosis, theranosis or the like from the samples according to methods of the invention; and (2) embodying the result of the determining step into a transmittable form. The transmittable form is the product of the production method. In one embodiment, a computer-readable medium includes a medium suitable for transmission of a result of an analysis of a biological sample, such as biosignatures. The medium can include a result regarding a vesicle, such as a biosignature of a subject, wherein such a result is derived using the methods described herein.

### Aptamer

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarity, hydrophobic contacts, steric exclusion) that drive affinity and specificity in antibody-antigen complexes.

Aptamers have a number of desirable characteristics for use as therapeutics and diagnostics including high specificity and affinity, biological efficacy, and excellent pharmacokinetic properties. In addition, they offer specific competitive advantages over antibodies and other protein biologics, for example:

Speed and control. Aptamers are produced by an entirely in vitro process, allowing for the rapid generation of initial leads, including therapeutic leads. In vitro selection allows the specificity and affinity of the aptamer to be tightly controlled and allows the generation of leads, including leads against both toxic and non-immunogenic targets.

Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be limited by immune response to antibodies themselves, it is more difficult to elicit host antibodies to aptamers, perhaps because aptamers cannot be presented by T-cells via the MHC and the immune response is generally trained not to recognize nucleic acid fragments.

Administration. Whereas most currently approved antibody therapeutics are administered by intravenous infusion (typically over 2-4 hours), aptamers can be administered by subcutaneous injection (aptamer bioavailability via subcutaneous administration is >80% in monkey studies (Tucker et al., J. Chromatography B. 732: 203-212, 1999)). This difference is primarily due to the comparatively low solubility and thus large volumes necessary for most therapeutic mAbs. With good solubility (>150 mg/mL) and comparatively low molecular weight (aptamer: 10-50 kDa; antibody: 150 kDa), a weekly dose of aptamer may be delivered by injection in a volume of less than 0.5 mL. In addition, the small size of aptamers allows them to penetrate into areas of conformational constrictions that do not allow for antibodies or antibody fragments to penetrate, presenting yet another advantage of aptamer-based therapeutics or prophylaxis.

Scalability and cost. Aptamers are chemically synthesized and are readily scaled as needed to meet production demand for diagnostic or therapeutic applications. Whereas difficulties in scaling production are currently limiting the availability of some biologics and the capital cost of a large-scale protein production plant is enormous, a single large-scale oligonucleotide synthesizer can produce upwards of 100 kg/year and requires a relatively modest initial investment. The current cost of goods for aptamer synthesis at the kilogram scale is estimated at $100/g, comparable to that for highly optimized antibodies.

Stability. Aptamers are chemically robust. They are intrinsically adapted to regain activity following exposure to factors such as heat and denaturants and can be stored for extended periods (>1 yr) at room temperature as lyophilized powders.

SELEX. A suitable method for generating an aptamer is with the process entitled “Systematic Evolution of Ligands by Exponential Enrichment” (“SELEX”) generally described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX-identified nucleic acid ligand, i.e., each aptamer, is a specific ligand of a given target compound or molecule. The SELEX process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

SELEX relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed and/or conserved sequence incorporated within randomized sequence. In other examples, the pool comprises random or partially random oligonucleotides containing at least one fixed and/or conserved sequence at its 5′ and/or 3′ end which may comprise a sequence shared by all the molecules of the oligonucleotide pool. Fixed sequences are sequences such as hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g. U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be for example, RNA, DNA, or RNA/DNA hybrid. In those instances where an RNA library is to be used as the starting library it is typically generated by transcribing a DNA library in vitro using T7 RNA polymerase or modified T7 RNA polymerases and purified. The library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process.

In one embodiment of SELEX, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX method has been modified to achieve a number of specific objectives. For example, U.S. Pat. No. 5,707,796 describes the use of SELEX in conjunction with gel electrophoresis to select nucleic acid molecules with specific structural characteristics, such as bent DNA. U.S. Pat. No. 5,763,177 describes SELEX based methods for selecting nucleic acid ligands containing photoreactive groups capable of binding and/or photocrosslinking to and/or photoinactivating a target molecule. U.S. Pat. No. 5,567,588 and U.S. Pat. No. 5,861,254 describe SELEX based methods which achieve highly efficient partitioning between oligonucleotides having high and low affinity for a target molecule. U.S. Pat. No. 5,496,938 describes methods for obtaining improved nucleic acid ligands after the SELEX process has been performed. U.S. Pat. No. 5,705,337 describes methods for covalently linking a ligand to its target.

SELEX can also be used to obtain nucleic acid ligands that bind to more than one site on the target molecule, and to obtain nucleic acid ligands that include non-nucleic acid species that bind to specific sites on the target. SELEX provides means for isolating and identifying nucleic acid ligands which bind to any envisionable target, including large and small biomolecules such as nucleic acid-binding proteins and proteins not known to bind nucleic acids as part of their biological function as well as cofactors and other small molecules. For example, U.S. Pat. No. 5,580,737 discloses nucleic acid sequences identified through SELEX which are capable of binding with high affinity to caffeine and the closely related analog, theophylline.

Counter-SELEX is a method for improving the specificity of nucleic acid ligands to a target molecule by eliminating nucleic acid ligand sequences with cross-reactivity to one or more non-target molecules. Counter-SELEX is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute intemucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX process modifications or post-SELEX process modifications (modification of previously identified unmodified ligands) or may be made by incorporation into the SELEX process.

Pre-SELEX process modifications or those made by incorporation into the SELEX process yield nucleic acid ligands with both specificity for their SELEX target and improved stability, e.g., in vivo stability. Post-SELEX process modifications made to nucleic acid ligands may result in improved stability, e.g., in vivo stability without adversely affecting the binding capacity of the nucleic acid ligand.

The SELEX method encompasses combining selected oligonucleotides with other selected oligonucleotides and non-oligonucleotide functional units as described in U.S. Pat. No. 5,637,459 and U.S. Pat. No. 5,683,867. The SELEX method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

The aptamers with specificity and binding affinity to the target(s) of the present invention are typically selected by the SELEX N process as described herein. As part of the SELEX process, the sequences selected to bind to the target are then optionally minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

2′ Modified SELEX. In order for an aptamer to be suitable for use as a therapeutic, it is preferably inexpensive to synthesize, safe and stable in vivo. Wild-type RNA and DNA aptamers are typically not stable is vivo because of their susceptibility to degradation by nucleases. Resistance to nuclease degradation can be greatly increased by the incorporation of modifying groups at the 2′-position.

Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis.

Aptamers that contain 2′-O-methyl (“2′-OMe”) nucleotides may overcome many of these drawbacks. Oligonucleotides containing 2′-OMe nucleotides are nuclease-resistant and inexpensive to synthesize. Although 2′-OMe nucleotides are ubiquitous in biological systems, natural polymerases do not accept 2′-OMe NTPs as substrates under physiological conditions, thus there are no safety concerns over the recycling of 2′-OMe nucleotides into host DNA. The SELEX method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-O-methyl substituted Nucleic Acids”, each of which is herein incorporated by reference in its entirety.

### Therapeutics

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent” according to the present invention, also includes a radiation therapy agent or a “chemotherapuetic agent.”

As used herein, the term “diagnostic agent” refers to any chemical used in the imaging of diseased tissue, such as, e.g., a tumor.

As used herein, the term “chemotherapuetic agent” refers to an agent with activity against cancer, neoplastic, and/or proliferative diseases, or that has ability to kill cancerous cells directly.

As used herein, “pharmaceutical formulations” include formulations for human and veterinary use with no significant adverse toxicological effect. “Pharmaceutically acceptable formulation” as used herein refers to a composition or formulation that allows for the effective distribution of the nucleic acid molecules of the instant invention in the physical location most suitable for their desired activity.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

### Therapeutic Aptamers

Previous work has developed the concept of antibody-toxin conjugates (“immunoconjugates”) as potential therapies for a range of indications, mostly directed at the treatment of cancer with a primary focus on hematological tumors. A variety of different payloads for targeted delivery have been tested in pre-clinical and clinical studies, including protein toxins, high potency small molecule cytotoxics, radioisotopes, and liposome-encapsulated drugs. While these efforts have successfully yielded three FDA-approved therapies for hematological tumors, immunoconjugates as a class (especially for solid tumors) have historically yielded disappointing results that have been attributable to multiple different properties of antibodies, including tendencies to develop neutralizing antibody responses to non-humanized antibodies, limited penetration in solid tumors, loss of target binding affinity as a result of toxin conjugation, and imbalances between antibody half-life and toxin conjugate half-life that limit the overall therapeutic index (reviewed by Reff and Heard, Critical Reviews in Oncology/Hematology, 40 (2001):25-35).

Aptamers are functionally similar to antibodies, except their absorption, distribution, metabolism, and excretion (“ADME”) properties are intrinsically different and they generally lack many of the immune effector functions generally associated with antibodies (e.g., antibody-dependent cellular cytotoxicity, complement-dependent cytotoxicity). In comparing many of the properties of aptamers and antibodies previously described, several factors suggest that aptamer therapeutics offers several concrete advantages over antibodies. Several potential advantages of aptamers over antibodies are as follows:

1) Aptamers are entirely chemically synthesized. Chemical synthesis provides more control over the nature of the therapeutic agent. Aptamers are also better able to be chemically modified. For example, stoichiometry (ratio of conjugates per aptamer) and site of attachment of conjugates can be precisely defined. Different linker chemistries can be readily tested. The reversibility of aptamer folding means that loss of activity during conjugation is unlikely and provides more flexibility in adjusting conjugation conditions to maximize yields.

2) Smaller size allows better tumor penetration. Poor penetration of antibodies into solid tumors is often cited as a factor limiting the efficacy of conjugate approaches. See Colcher, D., Goel, A., Pavlinkova, G., Beresford, G., Booth, B., Batra, S. K. (1999) “Effects of genetic engineering on the pharmacokinetics of antibodies,” Q. J. Nucl. Med., 43: 132-139. Studies comparing the properties of unPEGylated anti-tenascin C aptamers with corresponding antibodies demonstrate efficient uptake into tumors (as defined by the tumor:blood ratio) and evidence that aptamer localized to the tumor is unexpectedly long-lived (t₁₁₂>12 hours) (Hicke, B. J., Stephens, A. W., “Escort aptamers: a delivery service for diagnosis and therapy”, J. Clin. Invest., 106:923-928 (2000)).

3) Tunable PK. Aptamer half-life/metabolism can be tuned to match to optimize delivery to the target of interest while minimizing systemic exposure. Appropriate modifications to the aptamer backbone and addition of high molecular weight PEGs should make it possible to modulate the aptamer half-life.

4) Relatively low material requirements. It is likely that dosing levels will be limited by toxicity intrinsic to the cytotoxic payload. As such, a course of treatment will likely entail relatively small (<100 mg) quantities of aptamer, reducing the likelihood that the cost of oligonucleotide synthesis will be a barrier for aptamer-based therapies.

5) Parenteral administration is preferred for this indication. There will be no special need to develop alternative formulations to drive patient/physician acceptance.

To address the problem of immunosuppression resulting from a cancer, the invention further provides compositions and methods for inhibiting immunosuppressive factors produced by cancer cells both at their source and when secreted as microvesicles. Antibody therapies have been tested in animal models and early human trials with limited success. Often the host develops anti-idiotypic antibodies rendering such therapies ineffective. In addition, there can be many immunosuppressive factors related to cancer so blocking a single factor may not be sufficient to re-introduce an effective host immune response against the cancer. Thus, immunosuppressive pathways may compensate for the blocked immunosuppressive factor by such antibodies. The invention can address such multiple tumor-associated immunosuppressive factors secreted by the tumor.

The invention further provides compositions and methods for inhibiting immunosuppressive factor as well as stimulating the interacting host immune cells.

In an aspect, the invention provides therapeutic agents that bind to tumor-derived circulating microvesicles (cMVs). The therapeutic agents can inhibit an immunosuppressive factor on the cMVs and also stimulate the interacting immune cell to resist other immunosuppressive factors and support or induce anti-tumor immunity. Because cMVs may resemble their cell of origin regarding membrane structure, the therapeutic agent may further provide synergistic impact by inhibiting such immunosuppressive factors on the cancer cells themselves.

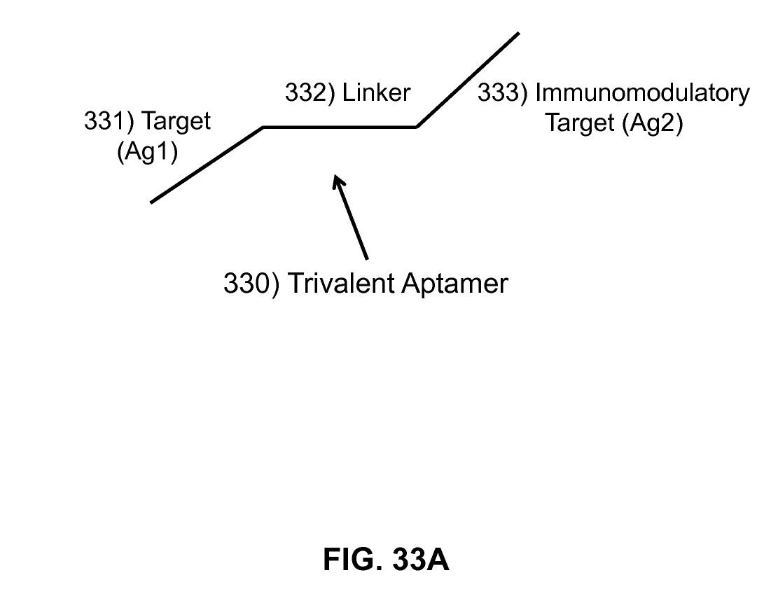

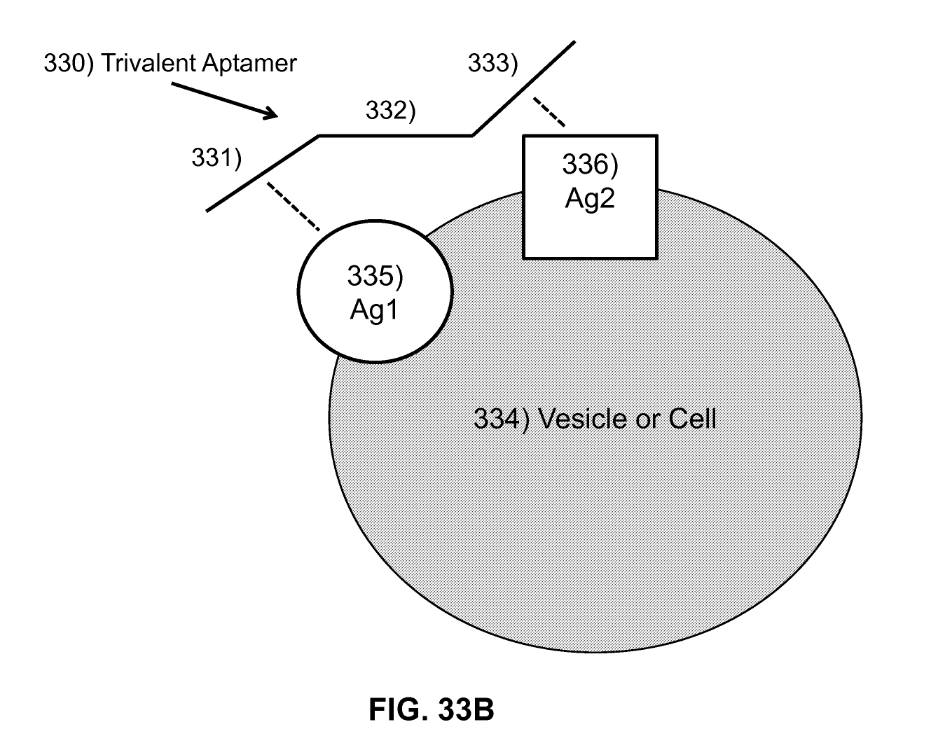

In an aspect, the therapeutic agent comprises a three component synthetic DNA oligonucleotide structure (also referred to herein a trivalent or tripartite aptamer). FIGS. 33A and 33B illustrate such tripartite aptamer 20. Aptamer 20 comprises: 1) a binding site 21 for a target of interest; 2) a binding site 23 for an immunosuppressive target; and 3) linker arm 22 between components 21 and 23. The target of interest 25 for region 21 may comprise a protein, such as a protein associated with cancer. In embodiment, the target protein comprises a membrane-associated protein indicative of a specific cancer type. The immunosuppressive target 26 can be a tumor-derived protein found on cMVs and/or cancer cells, including without limitation TGF-β, CD39, CD73, IL10, FasL and/or TRAIL. The immunosuppressive target 26 can be can be selected from the group consisting of FasL, programmed cell death 1 (PD-1), programmed death ligand-1 (PD-L1; B7-H1), programmed death ligand-2 (PD-L2; B7-DC), B7-H3, and/or B7-H4. The linker arm 22 can be chosen to allow target binding regions 21 and 23 to recognize their target on vesicle or cell 24 while minimizing or eliminating steric hindrance. The linker can be designed to have little to no biological effect or it can also be configured to provide beneficial effect. In an embodiment, the linker arm 22 comprises an immune-modulatory oligonucleotide. For example, the linker can be an oligonucleotide linker sequence including without limitation Toll-Like Receptor (TLR) agonists like CpG sequences which are immunostimulatory and/or polyG sequences which can be anti-proliferative or pro-apoptotic. The trivalent aptamer 20 can be optimized to selectively bind both cMVs and cells 24. For example, the aptamer 20 can bind both tumor-derived cMVs and cancer cells.

An alternate configuration of this invention consists of a chimeric oligonucleotide/fatty acid structure that functions as membrane pore forming complex which is able to integrate and disrupt cMVs and tumor cells in the patient. Such a structure would form a three dimensional structure with a hydrophobic center region flanked by hydrophilic regions. The hydrophobic will take on a ring-shaped structure to allow the passage of ions through the structure.

In an aspect, the invention provides a method of inhibiting or ameliorating a neoplastic growth. In an embodiment, the trivalent aptamer 20 is used to bind cMVs and/or cancer cells 24 in a cancer patient. The target binding region 21 can be configured to recognize a vesicle or cell of interest. The target so-recognized might be a cancer cell target, e.g., EpCam, CD24, Rab or B7H3, or the target may be a target for a cellular origin of interest, e.g., PCSA, PSMA or PBP in the case of prostatic cells. One of skill will immediately appreciate that any such target need not be 100% specific for an intended diseased cell or target to impart a beneficial effect. However, as desired or necessary, the target can be selected to maximize therapeutic effect while minimizing unintended binding. For example, EpCAM is not typically found in the circulation. Thus, EpCAM+ positive vesicles in circulation (EpCAM+cMVs) should primarily be released by diseased or otherwise damaged cells. Thus, an aptamer that binds EpCAM+ vesicles can be used to preferentially bind tumor-derived vesicles in the circulation of a cancer patient. The immunosuppressive target 26 recognized by aptamer region 23 can be selected to inhibit the immunosuppressive effects of the vesicles and therefore provide therapeutic benefit.

In one embodiment, the aptamer comprises an anti-EpCAM aptamer. For example, the target of interest 25 comprises EpCAM. The target of interest 25 can be selected from the vesicle proteins in Tables 3, 4 or 5 herein. In another embodiment, the target is selected from the group of proteins consisting of CD9, PSMA, PCSA, CD63, CD81, B7H3, IL 6, OPG-13, IL6R, PA2G4, EZH2, RUNX2, SERPINB3, and EpCam. In another embodiment, a target is selected from the group of proteins consisting of A33, a33 n15, AFP, ALA, ALIX, ALP, AnnexinV, APC, ASCA, ASPH (246-260), ASPH (666-680), ASPH (A-10), ASPH (D01P), ASPH (D03), ASPH (G-20), ASPH (H-300), AURKA, AURKB, B7H3, B7H4, BCA-225, BCNP1, BDNF, BRCA, CA125 (MUC16), CA-19-9, C-Bir, CD1.1, CD10, CD174 (Lewis y), CD24, CD44, CD46, CD59 (MEM-43), CD63, CD66e CEA, CD73, CD81, CD9, CDA, CDAC1 1a2, CEA, C-Erb2, C-erbB2, CRMP-2, CRP, CXCL12, CYFRA21-1, DLL4, DR3, EGFR, Epcam, EphA2, EphA2 (H-77), ER, ErbB4, EZH2, FASL, FRT, FRT c.f23, GDF15, GPCR, GPR30, Gro-alpha, HAP, HBD 1, HBD2, HER 3 (ErbB3), HSP, HSP70, hVEGFR2, iC3b, IL 6 Unc, IL-1B, IL6 Unc, IL6R, IL8, IL-8, INSIG-2, KLK2, L1CAM, LAMN, LDH, MACC-1, MAPK4, MART-1, MCP-1, M-CSF, MFG-E8, MIC1, MIF, MIS RII, MMG, MMP26, MMP7, MMP9, MS4A1, MUC1, MUC1 seq1, MUC1 seq11A, MUC17, MUC2, Ncam, NGAL, NPGP/NPFF2, OPG, OPN, p53, p53, PA2G4, PBP, PCSA, PDGFRB, PGP9.5, PIM1, PR (B), PRL, PSA, PSMA, PSME3, PTEN, R5-CD9 Tube 1, Reg IV, RUNX2, SCRN1, seprase, SERPINB3, SPARC, SPB, SPDEF, SRVN, STAT 3, STEAP1, TF (FL-295), TFF3, TGM2, TIMP-1, TIMP1, TIMP2, TMEM211, TMPRSS2, TNF-alpha, Trail-R2, Trail-R4, TrKB, TROP2, Tsg 101, TWEAK, UNC93A, VEGF A, and YPSMA-1. The target can be selected from the group of proteins consisting of 5T4, A33, ACTG1, ADAM10, ADAM15, AFP, ALA, ALDOA, ALIX, ALP, ALX4, ANCA, Annexin V, ANXA2, ANXA6, APC, APOA1, ASCA, ASPH, ATP1A1, AURKA, AURKB, B7H3, B7H4, BANK1, BASP1, BCA-225, BCNP1, BDNF, BRCA, C1orf58, C20orf114, C8B, CA125 (MUC16), CA-19-9, CAPZA1, CAV1, C-Bir, CCSA-2, CCSA-3&4, CD1.1, CD10, CD151, CD174 (Lewis y), CD24, CD2AP, CD37, CD44, CD46, CD53, CD59, CD63, CD66 CEA, CD73, CD81, CD82, CD9, CDA, CDAC1 1a2, CEA, C-Erbb2, CFL1, CFP, CHMP4B, CLTC, COTL1, CRMP-2, CRP, CRTN, CTNND1, CTSB, CTSZ, CXCL12, CYCS, CYFRA21-1, DcR3, DLL4, DPP4, DR3, EEF1A1, EGFR, EHD1, ENO1, EpCAM, EphA2, ER, ErbB4, EZH2, F11R, F2, F5, FAM125A, FASL, Ferritin, FNBP1L, FOLH1, FRT, GAL3, GAPDH, GDF15, GLB1, GPCR (GPR110), GPR30, GPX3, GRO-1, Gro-alpha, HAP, HBD 1, HBD2, HER 3 (ErbB3), HIST1H1C, HIST1H2AB, HNP1-3, HSP, HSP70, HSP90AB1, HSPA1B, HSPA8, hVEGFR2, iC3b, ICAM, IGSF8, IL 6, IL-1B, IL6R, IL8, IMP3, INSIG-2, ITGB1, ITIH3, JUP, KLK2, L1CAM, LAMN, LDH, LDHA, LDHB, LUM, LYZ, MACC-1, MAPK4, MART-1, MCP-1, M-CSF, MFGE8, MGAM, MGC20553, MIC1, MIF, MIS RII, MMG, MMP26, MMP7, MMP9, MS4A1, MUC1, MUC17, MUC2, MYH2, MYL6B, Ncam, NGAL, NME1, NME2, NNMT, NPGP/NPFF2, OPG, OPG-13, OPN, p53, PA2G4, PABPC1, PABPC4, PACSIN2, PBP, PCBP2, PCSA, PDCD6IP, PDGFRB, PGP9.5, PIM1, PR (B), PRDX2, PRL, PSA, PSCA, PSMA, PSMA1, PSMA2, PSMA4, PSMA6, PSMA7, PSMB1, PSMB2, PSMB3, PSMB4, PSMB5, PSMB6, PSMB8, PSME3, PTEN, PTGFRN, Rab-5b, Reg IV, RPS27A, RUNX2, SCRN1, SDCBP, seprase, Sept-9, SERINC5, SERPINB3, SERPINB3, SH3GL1, SLC3A2, SMPDL3B, SNX9, SPARC, SPB, SPDEF, SPON2, SPR, SRVN, SSX2, SSX4, STAT 3, STEAP, STEAP1, TACSTD1, TCN2, tetraspanin, TF (FL-295), TFF3, TGM2, THBS1, TIMP, TIMP1, TIMP2, TMEM211, TMPRSS2, TNF-alpha, TPA, TPI1, TPS, Trail-R2, Trail-R4, TrKB, TROP2, TROP2, Tsg 101, TUBB, TWEAK, UNC93A, VDAC2, VEGF A, VPS37B, YPSMA-1, YWHAG, YWHAQ, and YWHAZ. In another embodiment, the target is selected from the group of proteins consisting of 5T4, ACTG1, ADAM10, ADAM15, ALDOA, ANXA2, ANXA6, APOA1, ATP1A1, BASP1, C1orf58, C20orf114, C8B, CAPZA1, CAV1, CD151, CD2AP, CD59, CD9, CD9, CFL1, CFP, CHMP4B, CLTC, COTL1, CTNND1, CTSB, CTSZ, CYCS, DPP4, EEF1A1, EHD1, ENO1, F11R, F2, F5, FAM125A, FNBP1L, FOLH1, GAPDH, GLB1, GPX3, HIST1H1C, HIST1H2AB, HSP90AB1, HSPA1B, HSPA8, IGSF8, ITGB1, ITIH3, JUP, LDHA, LDHB, LUM, LYZ, MFGE8, MGAM, MMP9, MYH2, MYL6B, NME1, NME2, PABPC1, PABPC4, PACSIN2, PCBP2, PDCD6IP, PRDX2, PSA, PSMA, PSMA1, PSMA2, PSMA4, PSMA6, PSMA7, PSMB1, PSMB2, PSMB3, PSMB4, PSMB5, PSMB6, PSMB8, PTGFRN, RPS27A, SDCBP, SERINC5, SH3GL1, SLC3A2, SMPDL3B, SNX9, TACSTD1, TCN2, THBS1, TPI1, TSG101, TUBB, VDAC2, VPS37B, YWHAG, YWHAQ, and YWHAZ. In another embodiment, the target is selected from the group of proteins consisting of CD9, CD63, CD81, PSMA, PCSA, B7H3 and EpCam. CD9, CD63, CD81, PSMA, PCSA, B7H3 and EpCam. In another embodiment, the target is selected from the group of proteins consisting of a tetraspanin, CD9, CD63, CD81, CD63, CD9, CD81, CD82, CD37, CD53, Rab-5b, Annexin V, MFG-E8, Muc1, GPCR 110, TMEM211 and CD24 In another embodiment, the target is selected from the group of proteins consisting of A33, AFP, ALIX, ALX4, ANCA, APC, ASCA, AURKA, AURKB, B7H3, BANK1, BCNP1, BDNF, CA-19-9, CCSA-2, CCSA-3&4, CD10, CD24, CD44, CD63, CD66 CEA, CD66e CEA, CD81, CD9, CDA, C-Erb2, CRMP-2, CRP, CRTN, CXCL12, CYFRA21-1, DcR3, DLL4, DR3, EGFR, Epcam, EphA2, FASL, FRT, GAL3, GDF15, GPCR (GPR110), GPR30, GRO-1, HBD 1, HBD2, HNP1-3, IL-1B, IL8, IMP3, L1CAM, LAMN, MACC-1, MGC20553, MCP-1, M-CSF, MIC1, MIF, MMP7, MMP9, MS4A1, MUC1, MUC17, MUC2, Ncam, NGAL, NNMT, OPN, p53, PCSA, PDGFRB, PRL, PSMA, PSME3, Reg IV, SCRN1, Sept-9, SPARC, SPON2, SPR, SRVN, TFF3, TGM2, TIMP-1, TMEM211, TNF-alpha, TPA, TPS, Trail-R2, Trail-R4, TrKB, TROP2, Tsg 101, TWEAK, UNC93A, and VEGFA. In another embodiment, the target is selected from the group of proteins consisting of CD9, EGFR, NGAL, CD81, STEAP, CD24, A33, CD66E, EPHA2, Ferritin, GPR30, GPR110, MMP9, OPN, p53, TMEM211, TROP2, TGM2, TIMP, EGFR, DR3, UNC93A, MUC17, EpCAM, MUC1, MUC2, TSG101, CD63, B7H3, CD24, and a tetraspanin. The target can be selected from the group of proteins consisting of 5HT2B, 5T4 (trophoblast), ACO2, ACSL3, ACTN4, ADAM10, AGR2, AGR3, ALCAM, ALDH6A1, ANGPTL4, ANO9, AP1G1, APC, APEX1, APLP2, APP (Amyloid precursor protein), ARCN1, ARHGAP35, ARL3, ASAH1, ASPH (A-10), ATP1B1, ATP1B3, ATP5I, ATP5O, ATXN1, B7H3, BACE1, BAI3, BAIAP2, BCA-200, BDNF, BigH3, BIRC2, BLVRB, BRCA, BST2, C1GALT1, C1GALT1C1, C20orf3, CA125, CACYBP, Calmodulin, CAPN1, CAPNS1, CCDC64B, CCL2 (MCP-1), CCT3, CD10(BD), CD127 (IL7R), CD174, CD24, CD44, CD80, CD86, CDH1, CDH5, CEA, CFL2, CHCHD3, CHMP3, CHRDL2, CIB1, CKAP4, COPA, COX5B, CRABP2, CRIP1, CRISPLD1, CRMP-2, CRTAP, CTLA4, CUL3, CXCR3, CXCR4, CXCR6, CYB5B, CYB5R1, CYCS, CYFRA 21, DBI, DDX23, DDX39B, derlin 1, DHCR7, DHX9, DLD, DLL4, DNAJBL DPP6, DSTN, eCadherin, EEF1D, EEF2, EFTUD2, EIF4A2, EIF4A3, EpCaM, EphA2, ER(1) (ESR1), ER(2) (ESR2), Erb B4, Erb2, erb3 (Erb-B3?), ERLIN2, ESD, FARSA, FASN, FEN1, FKBP5, FLNB, FOXP3, FUS, Gal3, GCDPF-15, GCNT2, GNAl2, GNG5, GNPTG, GPC6, GPD2, GPER (GPR30), GSPT1, H3F3B, H3F3C, HADH, HAP1, HER3, HIST1H1C, HIST1H2AB, HIST1H3A, HIST1H3C, HIST1H3D, HIST1H3E, HIST1H3F, HIST1H3G, HIST1H3H, HIST1H3I, HIST1H3J, HIST2H2BF, HIST2H3A, HIST2H3C, HIST2H3D, HIST3H3, HMGB1, HNRNPA2B1, HNRNPAB, HNRNPC, HNRNPD, HNRNPH2, HNRNPK, HNRNPL, HNRNPM, HNRNPU, HPS3, HSP-27, HSP70, HSP90B1, HSPA1A, HSPA2, HSPA9, HSPE1, IC3b, IDE, IDH3B, IDO1, IFI30, IL1RL2, IL7, IL8, ILF2, ILF3, IQCG, ISOC2, IST1, ITGA7, ITGB7, junction plakoglobin, Keratin 15, KRAS, KRT19, KRT2, KRT7, KRT8, KRT9, KTN1, LAMP1, LMNA, LMNB1, LNPEP, LRPPRC, LRRC57, Mammaglobin, MAN1A1, MAN1A2, MART1, MATR3, MBD5, MCT2, MDH2, MFGE8, MFGE8, MGP, MMP9, MRP8, MUC1, MUC17, MUC2, MYO5B, MYOF, NAPA, NCAM, NCL, NG2 (CSPG4), Ngal, NHE-3, NME2, NONO, NPM1, NQO1, NT5E (CD73), ODC1, OPG, OPN (SC), 0S9, p53, PACSIN3, PAICS, PARK7, PARVA, PC, PCNA, PCSA, PD-1, PD-L1, PD-L2, PGP9.5, PHB, PHB2, PIK3C2B, PKP3, PPL, PR(B), PRDX2, PRKCB, PRKCD, PRKDC, PSA, PSAP, PSMA, PSMB7, PSMD2, PSME3, PYCARD, RAB1A, RAB3D, RAB7A, RAGE, RBL2, RNPEP, RPL14, RPL27, RPL36, RPS25, RPS4X, RPS4Y1, RPS4Y2, RUVBL2, SET, SHMT2, SLAIN1, SLC39A14, SLC9A3R2, SMARCA4, SNRPD2, SNRPD3, SNX33, SNX9, SPEN, SPR, SQSTM1, SSBP1, ST3GAL1, STXBP4, SUB1, SUCLG2, Survivin, SYT9, TFF3 (secreted), TGOLN2, THBS1, TIMP1, TIMP2, TMED10, TMED4, TMED9, TMEM211, TOM1, TRAF4 (scaffolding), TRAIL-R2, TRAP1, TrkB, Tsg 101, TXNDC16, U2AF2, UEVLD, UFC1, UNC93a, USP14, VASP, VCP, VDAC1, VEGFA, VEGFR1, VEGFR2, VPS37C, WIZ, XRCC5, XRCC6, YB-1, YWHAZ, or any combination thereof. In other embodiments, the target is selected from the group consisting of p53, p63, p73, mdm-2, procathepsin-D, B23, C23, PLAP, CA125, MUC-1, HER2, NY-ESO-1, SCP1, SSX-1, SSX-2, SSX-4, HSP27, HSP60, HSP90, GRP78, TAG72, HoxA7, HoxB7, EpCAM, ras, mesothelin, survivin, EGFK, MUC-1, or c-myc.

The aptamer of the invention can further comprise additional elements to add desired biological effects. For example, the aptamer may comprise an immunostimulatory moiety. In other embodiments, the aptamer may comprise a membrane disruptive moiety. For example, the aptamer may comprise an oligonucleotide sequence including without limitation Toll-Like Receptor (TLR) agonists like CpG sequences which are immunostimulatory and/or polyG sequences which can be anti-proliferative or pro-apoptotic. The aptamer may also be conjugated to one or more chemical moiety that provides such effects. For example, the aptamer may be conjugated to a detergent like moiety to disrupt the membrane of the target vesicle. Useful ionic detergents include sodium dodecyl sulfate (SDS, sodium lauryl sulfate (SLS)), sodium laureth sulfate (SLS, sodium lauryl ether sulfate (SLES)), ammonium lauryl sulfate (ALS), cetrimonium bromide, cetrimonium chloride, cetrimonium stearate, and the like. Useful non-ionic (zwitterionic) detergents include polyoxyethylene glycols, polysorbate 20 (also known as Tween 20), other polysorbates (e.g., 40, 60, 65, 80, etc), Triton-X (e.g., X100, X114), 3-[(3-cholamidopropyl)dimethylammonio]-1-propanesulfonate (CHAPS), CHAPSO, deoxycholic acid, sodium deoxycholate, NP-40, glycosides, octyl-thio-glucosides, maltosides, and the like. The moiety can be vaccine like moiety or antigen that stimulates an immune response. In an embodiment, the immune stimulating moiety comprises a superantigen. In some embodiments, the superantigen can be selected from the group consisting of staphylococcal enterotoxins (SEs), a Streptococcus pyogenes exotoxin (SPE), a Staphylococcus aureus toxic shock-syndrome toxin (TSST-1), a streptococcal mitogenic exotoxin (SME), a streptococcal superantigen (SSA), a hepatitis surface antigen, or a combination thereof. Other bacterial antigens that can be used with the invention comprise bacterial antigens such as Freund's complete adjuvant, Freund's incomplete adjuvant, monophosphoryl-lipid A/trehalose dicorynomycolate (Ribi's adjuvant), BCG (Calmette-Guerin Bacillus; Mycobacterium bovis), and Corynebacterium parvum. The immune stimulating moiety can also be a non-specific immunostimulant, such as an adjuvant or other non-specific immunostimulator. Useful adjuvants comprise without limitation aluminium salts, alum, aluminium phosphate, aluminium hydroxide, squalene, oils, MF59, and AS03 (“Adjuvant System 03”). The adjuvant can be selected from the group consisting of Cationic liposome-DNA complex JVRS-100, aluminum hydroxide vaccine adjuvant, aluminum phosphate vaccine adjuvant, aluminum potassium sulfate adjuvant, Alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, ASO4, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and Matrix-S. Additional adjuvants that can be used with the aptamers of the invention can be identified using the Vaxjo database. See Sayers S, Ulysse G, Xiang Z, and He Y. Vaxjo: a web-based vaccine adjuvant database and its application for analysis of vaccine adjuvants and their uses in vaccine development. Journal of Biomedicine and Biotechnology. 2012; 2012:831486. Epub 2012 Mar. 13. PMID: 22505817; www.violinet.org/vaxjo/. Other useful non-specific immunostimulators comprise histamine, interferon, transfer factor, tuftsin, interleukin-1, female sex hormones, prolactin, growth hormone vitamin D, deoxycholic acid (DCA), tetrachlorodecaoxide (TCDO), and imiquimod or resiquimod, which are drugs that activate immune cells through the toll-like receptor 7. One of skill will appreciate that functional fragments of the immunomodulating and/or membrance disruptive moieties can be covalently or non-covalently attached to the aptamer.

Pharmaceutical Compositions

In an aspect, the invention provides pharmaceutical compositions comprising the aptamers of the invention, e.g., the aptamers as described above. The invention further provides methods of administering such compositions.

The term “condition,” as used herein means an interruption, cessation, or disorder of a bodily function, system, or organ. Representative conditions include, but are not limited to, diseases such as cancer, inflammation, diabetes, and organ failure.

The phrase “treating,” “treatment of,” and the like include the amelioration or cessation of a specified condition.

The phrase “preventing,” “prevention of,” and the like include the avoidance of the onset of a condition.

The term “salt,” as used herein, means two compounds that are not covalently bound but are chemically bound by ionic interactions.

The term “pharmaceutically acceptable,” as used herein, when referring to a component of a pharmaceutical composition means that the component, when administered to an animal, does not have undue adverse effects such as excessive toxicity, irritation, or allergic response commensurate with a reasonable benefit/risk ratio. Accordingly, the term “pharmaceutically acceptable organic solvent,” as used herein, means an organic solvent that when administered to an animal does not have undue adverse effects such as excessive toxicity, irritation, or allergic response commensurate with a reasonable benefit/risk ratio. Preferably, the pharmaceutically acceptable organic solvent is a solvent that is generally recognized as safe (“GRAS”) by the United States Food and Drug Administration (“FDA”). Similarly, the term “pharmaceutically acceptable organic base,” as used herein, means an organic base that when administered to an animal does not have undue adverse effects such as excessive toxicity, irritation, or allergic response commensurate with a reasonable benefit/risk ratio.

The phrase “injectable” or “injectable composition,” as used herein, means a composition that can be drawn into a syringe and injected subcutaneously, intraperitoneally, or intramuscularly into an animal without causing adverse effects due to the presence of solid material in the composition. Solid materials include, but are not limited to, crystals, gummy masses, and gels. Typically, a formulation or composition is considered to be injectable when no more than about 15%, preferably no more than about 10%, more preferably no more than about 5%, even more preferably no more than about 2%, and most preferably no more than about 1% of the formulation is retained on a 0.22 μm filter when the formulation is filtered through the filter at 98° F. There are, however, some compositions of the invention, which are gels, that can be easily dispensed from a syringe but will be retained on a 0.22 μm filter. In one embodiment, the term “injectable,” as used herein, includes these gel compositions. In one embodiment, the term “injectable,” as used herein, further includes compositions that when warmed to a temperature of up to about 40° C. and then filtered through a 0.22 μm filter, no more than about 15%, preferably no more than about 10%, more preferably no more than about 5%, even more preferably no more than about 2%, and most preferably no more than about 1% of the formulation is retained on the filter. In one embodiment, an example of an injectable pharmaceutical composition is a solution of a pharmaceutically active compound (for example, an aptamer) in a pharmaceutically acceptable solvent. One of skill will appreciate that injectable solutions have inherent properties, e.g., sterility, pharmaceutically acceptable excipients and free of harmful measures of pyrogens or similar contaminants.

The term “solution,” as used herein, means a uniformly dispersed mixture at the molecular or ionic level of one or more substances (solute), in one or more other substances (solvent), typically a liquid.

The term “suspension,” as used herein, means solid particles that are evenly dispersed in a solvent, which can be aqueous or non-aqueous.

The term “animal,” as used herein, includes, but is not limited to, humans, canines, felines, equines, bovines, ovines, porcines, amphibians, reptiles, and avians. Representative animals include, but are not limited to a cow, a horse, a sheep, a pig, an ungulate, a chimpanzee, a monkey, a baboon, a chicken, a turkey, a mouse, a rabbit, a rat, a guinea pig, a dog, a cat, and a human. In one embodiment, the animal is a mammal. In one embodiment, the animal is a human. In one embodiment, the animal is a non-human. In one embodiment, the animal is a canine, a feline, an equine, a bovine, an ovine, or a porcine.

The phrase “drug depot,” as used herein means a precipitate, which includes the aptamer, formed within the body of a treated animal that releases the aptamer over time to provide a pharmaceutically effective amount of the aptamer.

The phrase “substantially free of,” as used herein, means less than about 2 percent by weight. For example, the phrase “a pharmaceutical composition substantially free of water” means that the amount of water in the pharmaceutical composition is less than about 2 percent by weight of the pharmaceutical composition.

The term “effective amount,” as used herein, means an amount sufficient to treat or prevent a condition in an animal.

The nucleotides that make up the aptamer can be modified to, for example, improve their stability, i.e., improve their in vivo half-life, and/or to reduce their rate of excretion when administered to an animal. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or 2′-azido-ribose; carbocyclic sugar analogues; α-anomeric sugars; and epimeric sugars such as arabinose, xyloses or lyxoses, pyranose sugars, furanose sugars, and sedoheptulose.

Modified nucleotides are known in the art and include, but are not limited to, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6-diaminopurine; methylpsuedouracil; 1-methylguanine; and 1-methylcytosine.

The aptamer can also be modified by replacing one or more phosphodiester linkages with alternative linking groups. Alternative linking groups include, but are not limited to embodiments wherein P(O)O is replaced by P(O)S, P(S)S, P(O)NR2, P(O)R, P(O)OR′, CO, or CH2, wherein each R or R′ is independently H or a substituted or unsubstituted C1-C20 alkyl. A preferred set of R substitutions for the P(O)NR2 group are hydrogen and methoxyethyl. Linking groups are typically attached to each adjacent nucleotide through an —O— bond, but may be modified to include —N— or —S— bonds. Not all linkages in an oligomer need to be identical.

The aptamer can also be modified by conjugating the aptamer to a polymer, for example, to reduce the rate of excretion when administered to an animal. For example, the aptamer can be “PEGylated,” i.e., conjugated to polyethylene glycol (“PEG”). In one embodiment, the PEG has an average molecular weight ranging from about 20 kD to 80 kD. Methods to conjugate an aptamer with a polymer, such PEG, are well known to those skilled in the art (See, e.g., Greg T. Hermanson, Bioconjugate Techniques, Academic Press, 1966).

The aptamers of the invention, e.g., such as described above, can be used in the pharmaceutical compositions disclosed herein or known in the art.

In one embodiment, the pharmaceutical composition further comprises a solvent.

In one embodiment, the solvent comprises water.

In one embodiment, the solvent comprises a pharmaceutically acceptable organic solvent. Any useful and pharmaceutically acceptable organic solvents can be used in the compositions of the invention.

In one embodiment, the pharmaceutical composition is a solution of the salt in the pharmaceutically acceptable organic solvent.

In one embodiment, the pharmaceutical composition comprises a pharmaceutically acceptable organic solvent and further comprises a phospholipid, a sphingomyelin, or phosphatidyl choline. Without wishing to be bound by theory, it is believed that the phospholipid, sphingomyelin, or phosphatidyl choline facilitates formation of a precipitate when the pharmaceutical composition is injected into water and can also facilitate controlled release of the aptamer from the resulting precipitate. Typically, the phospholipid, sphingomyelin, or phosphatidyl choline is present in an amount ranging from greater than 0 to 10 percent by weight of the pharmaceutical composition. In one embodiment, the phospholipid, sphingomyelin, or phosphatidyl choline is present in an amount ranging from about 0.1 to 10 percent by weight of the pharmaceutical composition. In one embodiment, the phospholipid, sphingomyelin, or phosphatidyl choline is present in an amount ranging from about 1 to 7.5 percent by weight of the pharmaceutical composition. In one embodiment, the phospholipid, sphingomyelin, or phosphatidyl choline is present in an amount ranging from about 1.5 to 5 percent by weight of the pharmaceutical composition. In one embodiment, the phospholipid, sphingomyelin, or phosphatidyl choline is present in an amount ranging from about 2 to 4 percent by weight of the pharmaceutical composition.

The pharmaceutical compositions can optionally comprise one or more additional excipients or additives to provide a dosage form suitable for administration to an animal. When administered to an animal, the aptamer containing pharmaceutical compositions are typically administered as a component of a composition that comprises a pharmaceutically acceptable carrier or excipient so as to provide the form for proper administration to the animal. Suitable pharmaceutical excipients are described in Remington's Pharmaceutical Sciences 1447-1676 (Alfonso R. Gennaro ed., 19th ed. 1995), incorporated herein by reference. The pharmaceutical compositions can take the form of solutions, suspensions, emulsion, tablets, pills, pellets, capsules, capsules containing liquids, powders, suppositories, emulsions, aerosols, sprays, suspensions, or any other form suitable for use.

In one embodiment, the pharmaceutical compositions are formulated for intravenous or parenteral administration. Typically, compositions for intravenous or parenteral administration comprise a suitable sterile solvent, which may be an isotonic aqueous buffer or pharmaceutically acceptable organic solvent. Where necessary, the compositions can also include a solubilizing agent. Compositions for intravenous administration can optionally include a local anesthetic such as lidocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where aptamer containing pharmaceutical compositions are to be administered by infusion, they can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

In another embodiment, the pharmaceutical compositions are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Oral compositions can include standard excipients such as mannitol, lactose, starch, magnesium stearate, sodium saccharin, cellulose, and magnesium carbonate. Typically, the excipients are of pharmaceutical grade. Orally administered compositions can also contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. A time-delay material such as glycerol monostearate or glycerol stearate can also be used.

The pharmaceutical compositions further comprising a solvent can optionally comprise a suitable amount of a pharmaceutically acceptable preservative, if desired, so as to provide additional protection against microbial growth. Examples of preservatives useful in the pharmaceutical compositions of the invention include, but are not limited to, potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chlorides (e.g., benzethonium chloride).

In one embodiment, the pharmaceutical compositions of the invention optionally contain a suitable amount of a pharmaceutically acceptable polymer. The polymer can increase the viscosity of the pharmaceutical composition. Suitable polymers for use in the compositions and methods of the invention include, but are not limited to, hydroxypropylcellulose, hydoxypropylmethylcellulose (HPMC), chitosan, polyacrylic acid, and polymethacrylic acid.

Typically, the polymer is present in an amount ranging from greater than 0 to 10 percent by weight of the pharmaceutical composition. In one embodiment, the polymer is present in an amount ranging from about 0.1 to 10 percent by weight of the pharmaceutical composition. In one embodiment, the polymer is present in an amount ranging from about 1 to 7.5 percent by weight of the pharmaceutical composition. In one embodiment, the polymer is present in an amount ranging from about 1.5 to 5 percent by weight of the pharmaceutical composition. In one embodiment, the polymer is present in an amount ranging from about 2 to 4 percent by weight of the pharmaceutical composition. In one embodiment, the pharmaceutical compositions of the invention are substantially free of polymers.

In one embodiment, any additional components added to the pharmaceutical compositions of the invention are designated as GRAS by the FDA for use or consumption by animals. In one embodiment, any additional components added to the pharmaceutical compositions of the invention are designated as GRAS by the FDA for use or consumption by humans.

The components of the pharmaceutical composition (the solvents and any other optional components) are preferably biocompatible and non-toxic and, over time, are simply absorbed and/or metabolized by the body.

As described above, the pharmaceutical compositions of the invention can further comprise a solvent.

In one embodiment, the solvent comprises water.

In one embodiment, the solvent comprises a pharmaceutically acceptable organic solvent.

In an embodiment, the aptamers are available as the salt of a metal cation, for example, as the potassium or sodium salt. These salts, however, may have low solubility in aqueous solvents and/or organic solvents, typically, less than about 25 mg/mL. The pharmaceutical compositions of the invention comprising (i) an amino acid ester or amino acid amide and (ii) a protonated aptamer, however, may be significantly more soluble in aqueous solvents and/or organic solvents. Without wishing to be bound by theory, it is believed that the amino acid ester or amino acid amide and the protonated aptamer form a salt, such as illustrated above, and the salt is soluble in aqueous and/or organic solvents.

Similarly, without wishing to be bound by theory, it is believed that the pharmaceutical compositions comprising (i) an aptamer; (ii) a divalent metal cation; and (iii) optionally a carboxylate, a phospholipid, a phosphatidyl choline, or a sphingomyelin form a salt, such as illustrated above, and the salt is soluble in aqueous and/or organic solvents.

In one embodiment, the concentration of the aptamer in the solvent is greater than about 2 percent by weight of the pharmaceutical composition. In one embodiment, the concentration of the aptamer in the solvent is greater than about 5 percent by weight of the pharmaceutical composition. In one embodiment, the concentration of the aptamer in the solvent is greater than about 7.5 percent by weight of the pharmaceutical composition. In one embodiment, the concentration of the aptamer in the solvent is greater than about 10 percent by weight of the pharmaceutical composition. In one embodiment, the concentration of the aptamer in the solvent is greater than about 12 percent by weight of the pharmaceutical composition. In one embodiment, the concentration of the aptamer in the solvent is greater than about 15 percent by weight of the pharmaceutical composition. In one embodiment, the concentration of the aptamer in the solvent is ranges from about 2 percent to 5 percent by weight of the pharmaceutical composition. In one embodiment, the concentration of the aptamer in the solvent is ranges from about 2 percent to 7.5 percent by weight of the pharmaceutical composition. In one embodiment, the concentration of the aptamer in the solvent ranges from about 2 percent to 10 percent by weight of the pharmaceutical composition. In one embodiment, the concentration of the aptamer in the solvent is ranges from about 2 percent to 12 percent by weight of the pharmaceutical composition. In one embodiment, the concentration of the aptamer in the solvent is ranges from about 2 percent to 15 percent by weight of the pharmaceutical composition. In one embodiment, the concentration of the aptamer in the solvent is ranges from about 2 percent to 20 percent by weight of the pharmaceutical composition.

Any pharmaceutically acceptable organic solvent can be used in the pharmaceutical compositions of the invention. Representative, pharmaceutically acceptable organic solvents include, but are not limited to, pyrrolidone, N-methyl-2-pyrrolidone, polyethylene glycol, propylene glycol (i.e., 1,3-propylene glycol), glycerol formal, isosorbid dimethyl ether, ethanol, dimethyl sulfoxide, tetraglycol, tetrahydrofurfuryl alcohol, triacetin, propylene carbonate, dimethyl acetamide, dimethyl formamide, dimethyl sulfoxide, and combinations thereof.

In one embodiment, the pharmaceutically acceptable organic solvent is a water soluble solvent. A representative pharmaceutically acceptable water soluble organic solvents is triacetin.

In one embodiment, the pharmaceutically acceptable organic solvent is a water miscible solvent. Representative pharmaceutically acceptable water miscible organic solvents include, but are not limited to, glycerol formal, polyethylene glycol, and propylene glycol.

In one embodiment, the pharmaceutically acceptable organic solvent comprises pyrrolidone. In one embodiment, the pharmaceutically acceptable organic solvent is pyrrolidone substantially free of another organic solvent.

In one embodiment, the pharmaceutically acceptable organic solvent comprises N-methyl-2-pyrrolidone. In one embodiment, the pharmaceutically acceptable organic solvent is N-methyl-2-pyrrolidone substantially free of another organic solvent.

In one embodiment, the pharmaceutically acceptable organic solvent comprises polyethylene glycol. In one embodiment, the pharmaceutically acceptable organic solvent is polyethylene glycol substantially free of another organic solvent.

In one embodiment, the pharmaceutically acceptable organic solvent comprises propylene glycol. In one embodiment, the pharmaceutically acceptable organic solvent is propylene glycol substantially free of another organic solvent.

In one embodiment, the pharmaceutically acceptable organic solvent comprises glycerol formal. In one embodiment, the pharmaceutically acceptable organic solvent is glycerol formal substantially free of another organic solvent.

In one embodiment, the pharmaceutically acceptable organic solvent comprises isosorbid dimethyl ether. In one embodiment, the pharmaceutically acceptable organic solvent is isosorbid dimethyl ether substantially free of another organic solvent.

In one embodiment, the pharmaceutically acceptable organic solvent comprises ethanol. In one embodiment, the pharmaceutically acceptable organic solvent is ethanol substantially free of another organic solvent.

In one embodiment, the pharmaceutically acceptable organic solvent comprises dimethyl sulfoxide. In one embodiment, the pharmaceutically acceptable organic solvent is dimethyl sulfoxide substantially free of another organic solvent.

In one embodiment, the pharmaceutically acceptable organic solvent comprises tetraglycol. In one embodiment, the pharmaceutically acceptable organic solvent is tetraglycol substantially free of another organic solvent.

In one embodiment, the pharmaceutically acceptable organic solvent comprises tetrahydrofurfuryl alcohol. In one embodiment, the pharmaceutically acceptable organic solvent is tetrahydrofurfuryl alcohol substantially free of another organic solvent.

In one embodiment, the pharmaceutically acceptable organic solvent comprises triacetin. In one embodiment, the pharmaceutically acceptable organic solvent is triacetin substantially free of another organic solvent.

In one embodiment, the pharmaceutically acceptable organic solvent comprises propylene carbonate. In one embodiment, the pharmaceutically acceptable organic solvent is propylene carbonate substantially free of another organic solvent.

In one embodiment, the pharmaceutically acceptable organic solvent comprises dimethyl acetamide. In one embodiment, the pharmaceutically acceptable organic solvent is dimethyl acetamide substantially free of another organic solvent.

In one embodiment, the pharmaceutically acceptable organic solvent comprises dimethyl formamide. In one embodiment, the pharmaceutically acceptable organic solvent is dimethyl formamide substantially free of another organic solvent.

In one embodiment, the pharmaceutically acceptable organic solvent comprises at least two pharmaceutically acceptable organic solvents.

In one embodiment, the pharmaceutically acceptable organic solvent comprises N-methyl-2-pyrrolidone and glycerol formal. In one embodiment, the pharmaceutically acceptable organic solvent is N-methyl-2-pyrrolidone and glycerol formal. In one embodiment, the ratio of N-methyl-2-pyrrolidone to glycerol formal ranges from about 90:10 to 10:90.

In one embodiment, the pharmaceutically acceptable organic solvent comprises propylene glycol and glycerol formal. In one embodiment, the pharmaceutically acceptable organic solvent is propylene glycol and glycerol formal. In one embodiment, the ratio of propylene glycol to glycerol formal ranges from about 90:10 to 10:90.

In one embodiment, the pharmaceutically acceptable organic solvent is a solvent that is recognized as GRAS by the FDA for administration or consumption by animals. In one embodiment, the pharmaceutically acceptable organic solvent is a solvent that is recognized as GRAS by the FDA for administration or consumption by humans.

In one embodiment, the pharmaceutically acceptable organic solvent is substantially free of water. In one embodiment, the pharmaceutically acceptable organic solvent contains less than about 1 percent by weight of water. In one embodiment, the pharmaceutically acceptable organic solvent contains less about 0.5 percent by weight of water. In one embodiment, the pharmaceutically acceptable organic solvent contains less about 0.2 percent by weight of water. Pharmaceutically acceptable organic solvents that are substantially free of water are advantageous since they are not conducive to bacterial growth. Accordingly, it is typically not necessary to include a preservative in pharmaceutical compositions that are substantially free of water. Another advantage of pharmaceutical compositions that use a pharmaceutically acceptable organic solvent, preferably substantially free of water, as the solvent is that hydrolysis of the aptamer is minimized Typically, the more water present in the solvent the more readily the aptamer can be hydrolyzed. Accordingly, aptamer containing pharmaceutical compositions that use a pharmaceutically acceptable organic solvent as the solvent can be more stable than aptamer containing pharmaceutical compositions that use water as the solvent.

In one embodiment, comprising a pharmaceutically acceptable organic solvent, the pharmaceutical composition is injectable.

In one embodiment, the injectable pharmaceutical compositions are of sufficiently low viscosity that they can be easily drawn into a 20 gauge and needle and then easily expelled from the 20 gauge needle. Typically, the viscosity of the injectable pharmaceutical compositions are less than about 1,200 cps. In one embodiment, the viscosity of the injectable pharmaceutical compositions are less than about 1,000 cps. In one embodiment, the viscosity of the injectable pharmaceutical compositions are less than about 800 cps. In one embodiment, the viscosity of the injectable pharmaceutical compositions are less than about 500 cps. Injectable pharmaceutical compositions having a viscosity greater than about 1,200 cps and even greater than about 2,000 cps (for example gels) are also within the scope of the invention provided that the compositions can be expelled through an 18 to 24 gauge needle.

In one embodiment, comprising a pharmaceutically acceptable organic solvent, the pharmaceutical composition is injectable and does not form a precipitate when injected into water.

In one embodiment, comprising a pharmaceutically acceptable organic solvent, the pharmaceutical composition is injectable and forms a precipitate when injected into water. Without wishing to be bound by theory, it is believed, for pharmaceutical compositions that comprise a protonated aptamer and an amino acid ester or amide, that the α-amino group of the amino acid ester or amino acid amide is protonated by the aptamer to form a salt, such as illustrated above, which is soluble in the pharmaceutically acceptable organic solvent but insoluble in water. Similarly, when the pharmaceutical composition comprises (i) an aptamer; (ii) a divalent metal cation; and (iii) optionally a carboxylate, a phospholipid, a phosphatidyl choline, or a sphingomyelin, it is believed that the components of the composition form a salt, such as illustrated above, which is soluble in the pharmaceutically acceptable organic solvent but insoluble in water. Accordingly, when the pharmaceutical compositions are injected into an animal, at least a portion of the pharmaceutical composition precipitates at the injection site to provide a drug depot. Without wishing to be bound by theory, it is believed that when the pharmaceutically compositions are injected into an animal, the pharmaceutically acceptable organic solvent diffuses away from the injection site and aqueous bodily fluids diffuse towards the injection site, resulting in an increase in concentration of water at the injection site, that causes at least a portion of the composition to precipitate and form a drug depot. The precipitate can take the form of a solid, a crystal, a gummy mass, or a gel. The precipitate, however, provides a depot of the aptamer at the injection site that releases the aptamer over time. The components of the pharmaceutical composition, i.e., the amino acid ester or amino acid amide, the pharmaceutically acceptable organic solvent, and any other components are biocompatible and non-toxic and, over time, are simply absorbed and/or metabolized by the body.

In one embodiment, comprising a pharmaceutically acceptable organic solvent, the pharmaceutical composition is injectable and forms liposomal or micellar structures when injected into water (typically about 500 μL are injected into about 4 mL of water). The formation of liposomal or micellar structures are most often formed when the pharmaceutical composition includes a phospholipid. Without wishing to be bound by theory, it is believed that the aptamer in the form of a salt, which can be a salt formed with an amino acid ester or amide or can be a salt with a divalent metal cation and optionally a carboxylate, a phospholipid, a phosphatidyl choline, or a sphingomyelin, that is trapped within the liposomal or micellar structure. Without wishing to be bound by theory, it is believed that when these pharmaceutically compositions are injected into an animal, the liposomal or micellar structures release the aptamer over time.

In one embodiment, the pharmaceutical composition further comprising a pharmaceutically acceptable organic solvent is a suspension of solid particles in the pharmaceutically acceptable organic solvent. Without wishing to be bound by theory, it is believed that the solid particles comprise a salt formed between the amino acid ester or amino acid amide and the protonated aptamer wherein the acidic phosphate groups of the aptamer protonates the amino group of the amino acid ester or amino acid amide, such as illustrated above, or comprises a salt formed between the aptamer; divalent metal cation; and optional carboxylate, phospholipid, phosphatidyl choline, or sphingomyelin, as illustrated above. Pharmaceutical compositions that are suspensions can also form drug depots when injected into an animal.

By varying the lipophilicity and/or molecular weight of the amino acid ester or amino acid amide it is possible to vary the properties of pharmaceutical compositions that include these components and further comprise an organic solvent. The lipophilicity and/or molecular weight of the amino acid ester or amino acid amide can be varied by varying the amino acid and/or the alcohol (or amine) used to form the amino acid ester (or amino acid amide). For example, the lipophilicity and/or molecular weight of the amino acid ester can be varied by varying the R1 hydrocarbon group of the amino acid ester. Typically, increasing the molecular weight of R1 increase the lipophilicity of the amino acid ester. Similarly, the lipophilicity and/or molecular weight of the amino acid amide can be varied by varying the R3 or R4 groups of the amino acid amide.

For example, by varying the lipophilicity and/or molecular weight of the amino acid ester or amino acid amide it is possible to vary the solubility of the aptamer in water, to vary the solubility of the aptamer in the organic solvent, vary the viscosity of the pharmaceutical composition comprising a solvent, and vary the ease at which the pharmaceutical composition can be drawn into a 20 gauge needle and then expelled from the 20 gauge needle.

Furthermore, by varying the lipophilicity and/or molecular weight of the amino acid ester or amino acid amide (i.e., by varying R1 of the amino acid ester or R3 and R4 of the amino acid amide) it is possible to control whether the pharmaceutical composition that further comprises an organic solvent will form a precipitate when injected into water. Although different aptamers exhibit different solubility and behavior, generally the higher the molecular weight of the amino acid ester or amino acid amide, the more likely it is that the salt of the protonated aptamer and the amino acid ester of the amide will form a precipitate when injected into water. Typically, when R1 of the amino acid ester is a hydrocarbon of about C16 or higher the pharmaceutical composition will form a precipitate when injected into water and when R1 of the amino acid ester is a hydrocarbon of about C12 or less the pharmaceutical composition will not form a precipitate when injected into water. Indeed, with amino acid esters wherein R1 is a hydrocarbon of about C12 or less, the salt of the protonated aptamer and the amino acid ester is, in many cases, soluble in water. Similarly, with amino acid amides, if the combined number of carbons in R3 and R4 is 16 or more the pharmaceutical composition will typically form a precipitate when injected into water and if the combined number of carbons in R3 and R4 is 12 or less the pharmaceutical composition will not form a precipitate when injected into water. Whether or not a pharmaceutical composition that further comprises a pharmaceutically acceptable organic solvent will form a precipitate when injected into water can readily be determined by injecting about 0.05 mL of the pharmaceutical composition into about 4 mL of water at about 98° F. and determining how much material is retained on a 0.22 μm filter after the composition is mixed with water and filtered. Typically, a formulation or composition is considered to be injectable when no more than 10% of the formulation is retained on the filter. In one embodiment, no more than 5% of the formulation is retained on the filter. In one embodiment, no more than 2% of the formulation is retained on the filter. In one embodiment, no more than 1% of the formulation is retained on the filter.

Similarly, in pharmaceutical compositions that comprise a protonated aptamer and a diester or diamide of aspartic or glutamic acid, it is possible to vary the properties of pharmaceutical compositions by varying the amount and/or lipophilicity and/or molecular weight of the diester or diamide of aspartic or glutamic acid. Similarly, in pharmaceutical compositions that comprise an aptamer; a divalent metal cation; and a carboxylate, a phospholipid, a phosphatidyl choline, or a sphingomyelin, it is possible to vary the properties of pharmaceutical compositions by varying the amount and/or lipophilicity and/or molecular weight of the carboxylate, phospholipid, phosphatidyl choline, or sphingomyelin.

Further, when the pharmaceutical compositions that further comprises an organic solvent form a depot when administered to an animal, it is also possible to vary the rate at which the aptamer is released from the drug depot by varying the lipophilicity and/or molecular weight of the amino acid ester or amino acid amide. Generally, the more lipophilic the amino acid ester or amino acid amide, the more slowly the aptamer is released from the depot. Similarly, when the pharmaceutical compositions that further comprises an organic solvent and also further comprise a carboxylate, phospholipid, phosphatidyl choline, sphingomyelin, or a diester or diamide of aspartic or glutamic acid and form a depot when administered to an animal, it is possible to vary the rate at which the aptamer is released from the drug depot by varying the amount and/or lipophilicity and/or molecular weight of the carboxylate, phospholipid, phosphatidyl choline, sphingomyelin, or the diester or diamide of aspartic or glutamic acid.

Release rates from a precipitate can be measured injecting about 50 μL of the pharmaceutical composition into about 4 mL of deionized water in a centrifuge tube. The time that the pharmaceutical composition is injected into the water is recorded as T=0. After a specified amount of time, T, the sample is cooled to about −9° C. and spun on a centrifuge at about 13,000 rpm for about 20 min. The resulting supernatant is then analyzed by HPLC to determine the amount of aptamer present in the aqueous solution. The amount of aptamer in the pellet resulting from the centrifugation can also be determined by collecting the pellet, dissolving the pellet in about 10 μL of methanol, and analyzing the methanol solution by HPLC to determine the amount of aptamer in the precipitate. The amount of aptamer in the aqueous solution and the amount of aptamer in the precipitate are determined by comparing the peak area for the HPLC peak corresponding to the aptamer against a standard curve of aptamer peak area against concentration of aptamer. Suitable HPLC conditions can be readily determined by one of ordinary skill in the art.

Methods of Treatment

The pharmaceutical compositions of the invention are useful in human medicine and veterinary medicine. Accordingly, the invention further relates to a method of treating or preventing a condition in an animal comprising administering to the animal an effective amount of the pharmaceutical composition of the invention.

In one embodiment, the invention relates to methods of treating a condition in an animal comprising administering to an animal in need thereof an effective amount of a pharmaceutical composition of the invention.

In one embodiment, the invention relates to methods of preventing a condition in an animal comprising administering to an animal in need thereof an effective amount of a pharmaceutical composition of the invention.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical. The mode of administration is left to the discretion of the practitioner. In some embodiments, administration will result in the release of the aptamer into the bloodstream.

In one embodiment, the method of treating or preventing a condition in an animal comprises administering to the animal in need thereof an effective amount of an aptamer by parenterally administering the pharmaceutical composition of the invention. In one embodiment, the pharmaceutical compositions are administered by infusion or bolus injection. In one embodiment, the pharmaceutical composition is administered subcutaneously.

In one embodiment, the method of treating or preventing a condition in an animal comprises administering to the animal in need thereof an effective amount of an aptamer by orally administering the pharmaceutical composition of the invention. In one embodiment, the composition is in the form of a capsule or tablet.

The pharmaceutical compositions can also be administered by any other convenient route, for example, topically, by absorption through epithelial or mucocutaneous linings (e.g., oral, rectal, and intestinal mucosa, etc.).

The pharmaceutical compositions can be administered systemically or locally.

The pharmaceutical compositions can be administered together with another biologically active agent.

In one embodiment, the animal is a mammal.

In one embodiment the animal is a human.

In one embodiment, the animal is a non-human animal.

In one embodiment, the animal is a canine, a feline, an equine, a bovine, an ovine, or a porcine.

The effective amount administered to the animal depends on a variety of factors including, but not limited to the type of animal being treated, the condition being treated, the severity of the condition, and the specific aptamer being administered. A treating physician can determine an effective amount of the pharmaceutical composition to treat a condition in an animal.

In one embodiment, the aptamer comprises an anti-EpCAM aptamer. For example, the target of interest comprises EpCAM. In another embodiment, the target is selected from the group of proteins consisting of a EGFR, PBP, EpCAM, and KLK2. In another embodiment, the target is selected from the group of proteins consisting of a tetraspanin, EpCam, CD9, PCSA, CD63, CD81, PSMA, B7H3, PSCA, ICAM, STEAP, KLK2, SSX2, SSX4, PBP, SPDEF, and EGFR. In another embodiment, the target is selected from the group of proteins consisting of CD9, PSMA, PCSA, CD63, CD81, B7H3, IL 6, OPG-13, IL6R, PA2G4, EZH2, RUNX2, SERPINB3, and EpCam. In another embodiment, a target is selected from the group of proteins consisting of A33, a33 n15, AFP, ALA, ALIX, ALP, AnnexinV, APC, ASCA, ASPH (246-260), ASPH (666-680), ASPH (A-10), ASPH (D01P), ASPH (D03), ASPH (G-20), ASPH (H-300), AURKA, AURKB, B7H3, B7H4, BCA-225, BCNP1, BDNF, BRCA, CA125 (MUC16), CA-19-9, C-Bir, CD1.1, CD10, CD174 (Lewis y), CD24, CD44, CD46, CD59 (MEM-43), CD63, CD66e CEA, CD73, CD81, CD9, CDA, CDAC1 1a2, CEA, C-Erb2, C-erbB2, CRMP-2, CRP, CXCL12, CYFRA21-1, DLL4, DR3, EGFR, Epcam, EphA2, EphA2 (H-77), ER, ErbB4, EZH2, FASL, FRT, FRT c.f23, GDF15, GPCR, GPR30, Gro-alpha, HAP, HBD 1, HBD2, HER 3 (ErbB3), HSP, HSP70, hVEGFR2, iC3b, IL 6 Unc, IL-1B, IL6 Unc, IL6R, IL8, IL-8, INSIG-2, KLK2, L1CAM, LAMN, LDH, MACC-1, MAPK4, MART-1, MCP-1, M-CSF, MFG-E8, MIC1, MIF, MIS RII, MMG, MMP26, MMP7, MMP9, MS4A1, MUC1, MUC1 seq1, MUC1 seq11A, MUC17, MUC2, Ncam, NGAL, NPGP/NPFF2, OPG, OPN, p53, p53, PA2G4, PBP, PCSA, PDGFRB, PGP9.5, PIM1, PR (B), PRL, PSA, PSMA, PSME3, PTEN, R5-CD9 Tube 1, Reg IV, RUNX2, SCRN1, seprase, SERPINB3, SPARC, SPB, SPDEF, SRVN, STAT 3, STEAP1, TF (FL-295), TFF3, TGM2, TIMP-1, TIMP1, TIMP2, TMEM211, TMPRSS2, TNF-alpha, Trail-R2, Trail-R4, TrKB, TROP2, Tsg 101, TWEAK, UNC93A, VEGF A, and YPSMA-1. In another embodiment, the target is selected from the group of proteins consisting of 5T4, ACTG1, ADAM10, ADAM15, ALDOA, ANXA2, ANXA6, APOA1, ATP1A1, BASP1, C1orf58, C20orf114, C8B, CAPZA1, CAV1, CD151, CD2AP, CD59, CD9, CD9, CFL1, CFP, CHMP4B, CLTC, COTL1, CTNND1, CTSB, CTSZ, CYCS, DPP4, EEF1A1, EHD1, ENO1, F11R, F2, F5, FAM125A, ENBP1L, FOLH1, GAPDH, GLB1, GPX3, HIST1H1C, HIST1H2AB, HSP90AB1, HSPA1B, HSPA8, IGSF8, ITGB1, ITIH3, JUP, LDHA, LDHB, LUM, LYZ, MFGE8, MGAM, MMP9, MYH2, MYL6B, NME1, NME2, PABPC1, PABPC4, PACSIN2, PCBP2, PDCD6IP, PRDX2, PSA, PSMA, PSMA1, PSMA2, PSMA4, PSMA6, PSMA7, PSMB1, PSMB2, PSMB3, PSMB4, PSMB5, PSMB6, PSMB8, PTGFRN, RPS27A, SDCBP, SERINC5, SH3GL1, SLC3A2, SMPDL3B, SNX9, TACSTD1, TCN2, THBS1, TPI1, TSG101, TUBB, VDAC2, VPS37B, YWHAG, YWHAQ, and YWHAZ. In another embodiment, the target is selected from the group of proteins consisting of CD9, CD63, CD81, PSMA, PCSA, B7H3 and EpCam. CD9, CD63, CD81, PSMA, PCSA, B7H3 and EpCam. In another embodiment, the target is selected from the group of proteins consisting of a tetraspanin, CD9, CD63, CD81, CD63, CD9, CD81, CD82, CD37, CD53, Rab-5b, Annexin V, MFG-E8, Muc1, GPCR 110, TMEM211 and CD24 In another embodiment, the target is selected from the group of proteins consisting of A33, AFP, ALIX, ALX4, ANCA, APC, ASCA, AURKA, AURKB, B7H3, BANK1, BCNP1, BDNF, CA-19-9, CCSA-2, CCSA-3&4, CD10, CD24, CD44, CD63, CD66 CEA, CD66e CEA, CD81, CD9, CDA, C-Erb2, CRMP-2, CRP, CRTN, CXCL12, CYFRA21-1, DcR3, DLL4, DR3, EGFR, Epcam, EphA2, FASL, FRT, GAL3, GDF15, GPCR (GPR110), GPR30, GRO-1, HBD 1, HBD2, HNP1-3, IL-1B, IL8, IMP3, L1CAM, LAMN, MACC-1, MGC20553, MCP-1, M-CSF, MIC1, MIF, MMP7, MMP9, MS4A1, MUC1, MUC17, MUC2, Ncam, NGAL, NNMT, OPN, p53, PCSA, PDGFRB, PRL, PSMA, PSME3, Reg IV, SCRN1, Sept-9, SPARC, SPON2, SPR, SRVN, TFF3, TGM2, TIMP-1, TMEM211, TNF-alpha, TPA, TPS, Trail-R2, Trail-R4, TrKB, TROP2, Tsg 101, TWEAK, UNC93A, and VEGFA. In another embodiment, the target is selected from the group of proteins consisting of CD9, EGFR, NGAL, CD81, STEAP, CD24, A33, CD66E, EPHA2, Ferritin, GPR30, GPR110, MMP9, OPN, p53, TMEM211, TROP2, TGM2, TIMP, EGFR, DR3, UNC93A, MUC17, EpCAM, MUC1, MUC2, TSG101, CD63, B7H3, CD24, and a tetraspanin.

The immunosuppressive target 26 can be a tumor-derived protein found on cMVs and/or cancer cells, including without limitation TGF-β, CD39, CD73, IL10, FasL or TRAIL.

In one embodiment, the aptamer is an aptamer that inhibits angiogenesis. In one embodiment, the aptamer is an aptamer that inhibits angiogenesis and the disease being treated is cancer. In one embodiment, the aptamer is an aptamer that inhibits angiogenesis and the disease being treated is a solid tumor.

The aptamer can be an aptamer that inhibits a neoplastic growth or a cancer. In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The compositions and methods of the invention can be used to treat these and other cancers.

Kits

In an aspect of the invention, a kit or package is provided comprising one or more aptamer of the invention. The invention also provides a kit comprising a reagent to carry out the methods of the invention. For example, the reagent can be one or more aptamer, buffer, blocker, enzyme, or combination thereof. In an embodiment, the reagent comprises one or more aptamer of the invention.

In an embodiment, the kit comprises a tripartite aptamer as described herein.

### REFERENCES

Greenberg et al., 1995. Prostate cancer in a transgenic mouse. Proc. Natl. Acad. Sci. USA, 92: 3439-3443.
Nadal et al., 2011. DNA Aptamers against the Lup and 1 Food Allergen, PLoS ONE 7(4)e35253.
Krill et al., 1997. A simple method for the isolation and culture of epithelial and stromal cells from benign and neoplastic prostates. Urology 49:981-8.
Aled Clayton (2012) Cancer cells use exosomes as tools to manipulate immunity and the microenvironment. Oncolmmunity 1(1):78-80.
Zhang et al. (2010) Exosomes and immune surveillance of neoplastic lesions: a review. Biotechnic & Histochemistry Early Online, 1-8.
Clayton et al. (2011) Cancer exosomes express CD39 and CD73, which suppress T cells through adenosine production. J. Immunol. 187:1-8.
Liu et al. (2006) Murine mammary carcinoma exosomes promote tumor growth by suppression of NK cell function. 176:1375-85.
Xiang et al. (2009) Induction of myeloid-derived suppressor cells by tumor exosomes. Int. J. Cancer 124(11):2621-33.
Xie et al. (2009) Tumor apoptotic bodies inhibit CTL responses and antitumor immunity via membrane-bound transforming growth factor-B1 inducing CD8+T-cell anergy and CD4+TR1 cell responses. Cancer Res. 69:7756-66.
Theresa Whiteside (2012) Disarming suppressor cells to improve immunotherapy. Cancer Immunol. Immunother. 61:283-288.
Gonzalez-Reyes et al. (2011) Study of TLR3, TLR4 and TLR9 in prostate carcinoma and their association with biochemical recurrence. Cancer Immunol. Immunother. 60:217-26.
Lipford et al. (2000) Poly-quanosine motifs costimulate antigen-reactive CD8 T cells while bacterial CpG-DNA affect T-cell activation via antigen-presenting cell-derived cytokines. Immunology 101:46-52.
Wang et al. (2010) Antitumor activity of decoy oligodeoxynucleotides targeted to NF-kB in vitro and in vivo. Asian Pacific J. Cancer Prev. 10:193-9.
Nolte-'t Hoen and Wauben (2012) Immune cell-derived vesicles: modulators and mediators of inflammation. Curr Pharmaceutical Design 18(2357-68).
Klinman et al. (1996) CpG motifs present in bacterial DNA rapidly induce lymphocytes to secrete interleukin 6, interleukin 12 and interferon γ. PNAS USA 93:2879-83.
Weiner et al. (1997) Immunostimulatory oligodeoxynucleotides containing CpG motif are effective as immune adjuvants in tumor antigen immunization. PNAS USA 94:10833-37.
Dapic et al, 2003. Biophysical and biological properties of quadruplex oligodeoxyribonucleotides Nucleic Acid Research, 31(8):2097-2107.
Balasubramanian et al. (1998) Interferon-gamma-inhibitory oligodeoxynucleotides alter the conformation of interferon-gamma. Mol. Pharmacol. 53:926-32.
Lee et al. (1996) An oligonucleotide blocks interferon-gamma signal transduction. Transplantation 62:1297-1301.
Bishop et al. (1996) Intramolecular G-quartet motifs canfer nuclease resistance to a potent anti-HIV oligonucleotide. J. Biol. Chem. 271:5698-5703.
Wang et al. (1998) A comparison of guanosine-quartet inhibitory effects versus cytidine homopolymer inhibitory effects on rat neointimal formation. Antisense Nucleic Acid Drug Dev. 8:227-36.
Williamson et al. (1989) Monovalent cation-induced structure of telomeric DNA: the G-quartet model. Cell 59:871-80.
Ballas et al. (2001) Divergent therapeutic and immunologic effects of oligodeoxynucleotides with distinctive CpG motifs. J. Immunol. 167:4878-86.
Shen et al. (2002) Antitumor mechanisms of oligodeoxynucleotides with CpG and PolyG motifs in murine prostate cancer cells: decrease of NF-kB and AP-1 binding activities and induction of apoptosis. Antisense and Nucleic Acid Drug Dev. 12:155-64.
Ciavarra et al., 2000. Flt3-Ligand Induces Transient Tumor Regression in an Ectopic Treatment Model of Major Histocompatibility Complex-negative Prostate Cancer. Cancer Research 60:2081-2084.

### EXAMPLES

### Example 1

### Purification of Vesicles from Prostate Cancer Cell Lines

Prostate cancer cell lines are cultured for 3-4 days in culture media containing 20% FBS (fetal bovine serum) and 1% P/S/G. The cells are then pre-spun for 10 minutes at 400×g at 4° C. The supernatant is kept and centrifuged for 20 minutes at 2000×g at 4. The supernatant containing vesicles can be concentrated using a Millipore Centricon Plus-70 (Cat # UFC710008 Fisher).

The Centricon is pre washed with 30mls of PBS at 1000×g for 3 minutes at room temperature. Next, 15-70 mls of the pre-spun cell culture supernatant is poured into the Concentrate Cup and is centrifuged in a Swing Bucket Adapter (Fisher Cat #75-008-144) for 30 minutes at 1000×g at room temperature.

The flow through in the Collection Cup is poured off. The volume in the Concentrate Cup is brought back up to 60mls with any additional supernatant. The Concentrate Cup is centrifuged for 30 minutes at 1000×g at room temperature to concentrate the cell supernatant.

The Concentrate Cup is washed by adding 70 mls of PBS and centrifuged for 30-60 minutes at 1000×g until approximately 2 mls remains. The vesicles are removed from the filter by inverting the concentrate into the small sample cup and centrifuge for 1 minute at 4° C. The volume is brought up to 25 mls with PBS. The vesicles are now concentrated and are added to a 30% Sucrose Cushion.

To make a cushion, 4 mls of Tris/30% Sucrose/D20 solution (30 g protease-free sucrose, 2.4 g Tris base, 50 ml D20, adjust pH to 7.4 with 10N NCL drops, adjust volume to 100mls with D20, sterilize by passing thru a 0.22-um filter) is loaded to the bottom of a 30 ml V bottom thin walled Ultracentrifuge tube. The diluted 25 mls of concentrated vesicles is gently added above the sucrose cushion without disturbing the interface and is centrifuged for 75 minutes at 100,000×g at 4° C. The ˜25mls above the sucrose cushion is carefully removed with a 10 ml pipet and the ˜3.5mls of vesicles is collected with a fine tip transfer pipet (SAMCO 233) and transferred to a fresh ultracentrifuge tube, where 30 mls PBS is added. The tube is centrifuged for 70 minutes at 100,000×g at 4° C. The supernatant is poured off carefully. The pellet is resuspended in 200 ul PBS and can be stored at 4° C. or used for assays. A BCA assay (1:2) can be used to determine protein content and Western blotting or electron micrography can be used to determine vesicle purification.

### Example 2

### Purification of Vesicles from VCaP and 22Rv1

Vesicles from Vertebral-Cancer of the Prostate (VCaP) and 22Rv1, a human prostate carcinoma cell line, derived from a human prostatic carcinoma xenograft (CWR22R) were collected by ultracentrifugation by first diluting plasma with an equal volume of PBS (1 ml). The diluted fluid was transferred to a 15 ml falcon tube and centrifuged 30 minutes at 2000×g 4° C. The supernatant (˜2 mls) was transferred to an ultracentrifuge tube 5.0 ml PA thinwall tube (Sorvall #03127) and centrifuged at 12,000×g, 4° C. for 45 minutes.

The supernatant (˜2 mls) was transferred to a new 5.0 ml ultracentrifuge tubes and filled to maximum volume with addition of 2.5 mls PBS and centrifuged for 90 minutes at 110,000×g at 4° C. The supernatant was poured off without disturbing the pellet and the pellet resuspended with 1 ml PBS. The tube was filled to maximum volume with addition of 4.5 ml of PBS and centrifuged at 110,000×g, 4° C. for 70 minutes.

The supernatant was poured off without disturbing the pellet and an additional 1 ml of PBS was added to wash the pellet. The volume was increased to maximum volume with the addition of 4.5 mls of PBS and centrifuged at 110,000×g for 70 minutes at 4° C. The supernatant was removed with P-1000 pipette until ˜100 μl of PBS was in the bottom of the tube. The 90 μl remaining was removed with P-200 pipette and the pellet collected with the ˜10 μl of PBS remaining by gently pipetting using a P-20 pipette into the microcentrifuge tube. The residual pellet was washed from the bottom of a dry tube with an additional 5 μl of fresh PBS and collected into microcentrifuge tube and suspended in phosphate buffered saline (PBS) to a concentration of 500 μg/ml.

### Example 3

### Plasma Collection and Vesicle Purification

Blood is collected via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

For storage, approximately 1 ml of the plasma (supernatant) is aliquoted to a cryovials, placed in dry ice to freeze them and stored in −80° C. Before vesicle purification, if samples were stored at −80° C., samples are thawed in a cold water bath for 5 minutes. The samples are mixed end over end by hand to dissipate insoluble material.

In a first prespin, the plasma is diluted with an equal volume of PBS (example, approximately 2 ml of plasma is diluted with 2 ml of PBS). The diluted fluid is transferred to a 15 ml Falcon tube and centrifuged for 30 minutes at 2000×g at 4° C.

For a second prespin, the supernatant (approximately 4 mls) is carefully transferred to a 50 ml Falcon tube and centrifuged at 12,000×g at 4° C. for 45 minutes in a Sorval.

In the isolation step, the supernatant (approximately 2 mls) is carefully transferred to a 5.0 ml ultracentrifuge PA thinwall tube (Sorvall #03127) using a P1000 pipette and filled to maximum volume with an additional 0.5 mls of PBS. The tube is centrifuged for 90 minutes at 110,000×g at 4° C.

In the first wash, the supernatant is poured off without disturbing the pellet. The pellet is resuspended or washed with 1 ml PBS and the tube is filled to maximum volume with an additional 4.5 ml of PBS. The tube is centrifuged at 110,000×g at 4° C. for 70 minutes. A second wash is performed by repeating the same steps.

The vesicles are collected by removing the supernatant with P-1000 pipette until approximately 100 of PBS is in the bottom of the tube. Approximately 90 μl of the PBS is removed and discarded with P-200 pipette. The pellet and remaining PBS is collected by gentle pipetting using a P-20 pipette. The residual pellet is washed from the bottom of the dry tube with an additional 5 μl of fresh PBS and collected into a microcentrifuge tube.

### Example 4

### Analysis of Vesicles Using Antibody-Coupled Microspheres and Directly Conjugated Antibodies

This example demonstrates the use of particles coupled to an antibody, where the antibody captures the vesicles. See, e.g., FIG. 2B. An antibody, the detector antibody, is directly coupled to a label, and is used to detect a biomarker on the captured vesicle.

First, an antibody-coupled microsphere set is selected (Luminex, Austin, Tex.). The microsphere set can comprise various antibodies, and thus allows multiplexing. The microspheres are resuspended by vortex and sonication for approximately 20 seconds. A Working Microsphere Mixture is prepared by diluting the coupled microsphere stocks to a final concentration of 100 microspheres of each set/μL in Startblock (Pierce (37538)). 50 RL of Working Microsphere Mixture is used for each well. Either PBS-1% BSA or PBS-BN (PBS, 1% BSA, 0.05% Azide, pH 7.4) may be used as Assay Buffer.

A 1.2 μm Millipore filter plate is pre-wet with 100 μl/well of PBS-1% BSA (Sigma (P3688-10PAK+0.05% NaAzide (S8032))) and aspirated by vacuum manifold. An aliquot of 50 μl of the Working Microsphere Mixture is dispensed into the appropriate wells of the filter plate (Millipore Multiscreen HTS (MSBVN1250)). A 50 μl aliquot of standard or sample is dispensed into to the appropriate wells. The filter plate is covered and incubated for 60 minutes at room temperature on a plate shaker. The plate is covered with a sealer, placed on the orbital shaker and set to 900 for 15-30 seconds to re-suspend the beads. Following that the speed is set to 550 for the duration of the incubation.

The supernatant is aspirated by vacuum manifold (less than 5 inches Hg in all aspiration steps). Each well is washed twice with 100 μl of PBS-1% BSA (Sigma (P3688-10PAK+0.05% NaAzide (S8032))) and is aspirated by vacuum manifold. The microspheres are resuspended in 50 μL of PBS-1% BSA (Sigma (P3688-10PAK+0.05% NaAzide (S8032))). The phycoerythrin (PE) conjugated detection antibody is diluted to 4 μg/mL (or appropriate concentration) in PBS-1% BSA (Sigma (P3688-10PAK+0.05% NaAzide (S8032))). (Note: 50 RL of diluted detection antibody is required for each reaction.) A 50 μl aliquot of the diluted detection antibody is added to each well. The filter plate is covered and incubated for 60 minutes at room temperature on a plate shaker. The filter plate is covered with a sealer, placed on the orbital shaker and set to 900 for 15-30 seconds to re-suspend the beads. Following that the speed is set to 550 for the duration of the incubation. The supernatant is aspirated by vacuum manifold. The wells are washed twice with 100 μl of PBS-1% BSA (Sigma (P3688-10PAK+0.05% NaAzide (S8032))) and aspirated by vacuum manifold. The microspheres are resuspended in 100 μl of PBS-1% BSA (Sigma (P3688-10PAK+0.05% NaAzide (S8032))). The microspheres are analyzed on a Luminex analyzer according to the system manual.

### Example 5

### Analysis of Vesicles Using Antibody-Coupled Microspheres and Biotinylated Antibody

This example demonstrates the use of particles coupled to an antibody, where the antibody captures the vesicles. An antibody, the detector antibody, is biotinylated. A label coupled to streptavidin is used to detect the biomarker.

First, the appropriate antibody-coupled microsphere set is selected (Luminex, Austin, Tex.). The microspheres are resuspended by vortex and sonication for approximately 20 seconds. A Working Microsphere Mixture is prepared by diluting the coupled microsphere stocks to a final concentration of 50 microspheres of each set/μL in Startblock (Pierce (37538)). (Note: 50 μl of Working Microsphere Mixture is required for each well.) Beads in Start Block should be blocked for 30 minutes and no more than 1 hour.

A 1.2 nm Millipore filter plate is pre-wet with 100 μl/well of PBS-1% BSA+Azide (PBS-BN)((Sigma (P3688-10PAK+0.05% NaAzide (S8032))) and is aspirated by vacuum manifold. A 50 μl aliquot of the Working Microsphere Mixture is dispensed into the appropriate wells of the filter plate (Millipore Multiscreen HTS (MSBVN1250)). A 50 μl aliquot of standard or sample is dispensed to the appropriate wells. The filter plate is covered with a seal and is incubated for 60 minutes at room temperature on a plate shaker. The covered filter plate is placed on the orbital shaker and set to 900 for 15-30 seconds to re-suspend the beads. Following that, the speed is set to 550 for the duration of the incubation.

The supernatant is aspirated by a vacuum manifold (less than 5 inches Hg in all aspiration steps). Aspiration can be done with the Pall vacuum manifold. The valve is place in the full off position when the plate is placed on the manifold. To aspirate slowly, the valve is opened to draw the fluid from the wells, which takes approximately 3 seconds for the 100 μl of sample and beads to be fully aspirated from the well. Once the sample drains, the purge button on the manifold is pressed to release residual vacuum pressure from the plate.

Each well is washed twice with 100 μl of PBS-1% BSA+Azide (PBS-BN)(Sigma (P3688-10PAK+0.05% NaAzide (S8032))) and is aspirates by vacuum manifold. The microspheres are resuspended in 50 μl of PBS-1% BSA+Azide (PBS-BN)((Sigma (P3688-10PAK+0.05% NaAzide (S8032)))

The biotinylated detection antibody is diluted to 4 μg/mL in PBS-1% BSA+Azide (PBS-BN)((Sigma (P3688-10PAK+0.05% NaAzide (S8032))). (Note: 50 μl of diluted detection antibody is required for each reaction.) A 50 μl aliquot of the diluted detection antibody is added to each well.

The filter plate is covered with a sealer and is incubated for 60 minutes at room temperature on a plate shaker. The plate is placed on the orbital shaker and set to 900 for 15-30 seconds to re-suspend the beads. Following that, the speed is set to 550 for the duration of the incubation.

The supernatant is aspirated by vacuum manifold. Aspiration can be done with the Pall vacuum manifold. The valve is place in the full off position when the plate is placed on the manifold. To aspirate slowly, the valve is opened to draw the fluid from the wells, which takes approximately 3 seconds for the 100 ul of sample and beads to be fully aspirated from the well. Once all of the sample is drained, the purge button on the manifold is pressed to release residual vacuum pressure from the plate.

Each well is washed twice with 100 μl of PBS-1% BSA+Azide (PBS-BN)((Sigma (P3688-10PAK+0.05% NaAzide (S8032))) and is aspirated by vacuum manifold. The microspheres are resuspended in 50 μl of PBS-1% BSA (Sigma (P3688-10PAK+0.05% NaAzide (S8032))).

The streptavidin-R-phycoerythrin reporter (Molecular Probes 1 mg/ml) is diluted to 4 μg/mL in PBS-1% BSA+Azide (PBS-BN). 50 μl of diluted streptavidin-R-phycoerythrin was used for each reaction. A 50 μl aliquot of the diluted streptavidin-R-phycoerythrin is added to each well.

The filter plate is covered with a sealer and is incubated for 60 minutes at room temperature on a plate shaker. The plate is placed on the orbital shaker and set to 900 for 15-30 seconds to re-suspend the beads. Following that, the speed is set to 550 for the duration of the incubation.

The supernatant is aspirated by vacuum manifold. Aspiration can be done with the Pall vacuum manifold. The valve is place in the full off position when the plate is placed on the manifold. To aspirate slowly, the valve is opened to draw the fluid from the wells, which takes approximately 3 seconds for the 100 ul of sample and beads to be fully aspirated from the well. Once all of the sample is drained, the purge button on the manifold is pressed to release residual vacuum pressure from the plate.

Each well is washed twice with 100 μl of PBS-1% BSA+Azide (PBS-BN)((Sigma (P3688-10PAK+0.05% NaAzide (S8032))) and is aspirated by vacuum manifold. The microspheres are resuspended in 100 μl of PBS-1% BSA+Azide (PBS-BN)((Sigma (P3688-10PAK+0.05% NaAzide (S8032))) and analyzed on the Luminex analyzer according to the system manual.

### Example 6

### Vesicle Concentration from Plasma

Supplies and Equipment: Pall life sciences Acrodisc, 25 mm syringe filter w/1.2 um, Versapor membrane (sterile) Part number: 4190; Pierce concentrators 7 ml/150 K MWCO (molecular weight cut off), Part number: 89922; BD syringe filter, 10 ml, Part number: 305482; Sorvall Legend RT Plus Series Benchtop Centrifuge w 15 ml swinging bucket rotor; PBS, pH 7.4, Sigma cat#P3813-10PAK prepared in Sterile Molecular grade water; Co-polymer 1.7 ml microfuge tubes, USA Scientific, cat#1415-2500. Water used for reagents is Sterile Filtered Molecular grade water (Sigma, cat#W4502). Handling of patient plasma is done in a biosafety hood.

### Procedure:

1. Filter procedure for plasma samples
1.1. Remove plasma samples from −80° C. (˜65° C. to −85° C.) freezer
1.2. Thaw samples in room temperature water (10-15 minutes).
1.3. Prepare syringe and filter by removing the number necessary from their casing.
1.4. Pull plunger to draw 4 mL of sterile molecular grade water into the syringe. Attach a 1.2 μm filter to the syringe tip and pass contents through the filter onto the 7 ml/150 K MWCO Pierce column.
1.5. Cap the columns and place in the swing bucket centrifuge at spin at 1000×g in Sorvall Legend RT plus centrifuge for 4 minutes at 20° C. (16° C.-24° C.).
1.6. While spinning, disassemble the filter from syringe. Then remove plunger from syringe.
1.7. Discard flow through from the tube and gently tap column on paper towels to remove any residual water.
1.8. Measure and record starting volumes for all plasma samples. Samples with a volume less than 900 μl may not be processed.
1.9. Place open syringe and filter on open Pierce column. Fill open end of syringe with 5.2 mL of 1×PBS and pipette mix plasma into PBS three to four times.
1.10. Replace the plunger of the syringe and slowly depress the plunger until the contents of the syringe have passed through the filter onto the Pierce column. Contents should pass through the filter drop wise.

### 2. Microvesicle Concentration Centrifugation Protocol

2.1. Spin 7 ml/150 K MWCO Pierce columns at 2000×g at 20° C. (16° C.-24° C.) for 60 minutes or until volume is reduced to 250-300 μL. If needed, spin for additional 15 minutes increments to reach required volume.
2.2. At the conclusion of the spin, pipette mix on the column 15× (avoid creating bubbles) and withdraw volume (300 μL or less) and transfer to a new 1.7 mL co-polymer tube.
2.3. The final volume of the plasma concentrate is dependent on the initial volume of plasma. Plasma is concentrated to 300 ul if the original plasma volume is 1 ml. If the original volume of plasma is less than 1 ml, then the volume of concentrate should be consistent with that ratio. For example, if the original volume is 900 ul, then the volume of concentrate is 270 ul. The equation to follow is: x=(y/1000)*300, where x is the final volume of concentrate and y is the initial volume of plasma.
2.4. Record the sample volume and add 1×PBS to the sample to make the final sample volume.
2.5. Store concentrated microvesicle sample at 4° C. (2° C. to 8° C.).

### Calculations:

1. Final volume of concentrated plasma sample
   x=(y/1000)*300, where x is the final volume of concentrate and y is the initial volume of plasma.

### Example 7

### Capture of Vesicles Using Magnetic Beads

Vesicles isolated as described in Example 2 are used. Approximately 40 μl of the vesicles are incubated with approximately 5 ng (˜50 μl) of EpCam antibody coated Dynal beads (Invitrogen, Carlsbad, Calif.) and 50 μl of Starting Block. The vesicles and beads are incubated with shaking for 2 hours at 45° C. in a shaking incubator. The tube containing the Dynal beads is placed on the magnetic separator for 1 minute and the supernatant removed. The beads are washed twice and the supernatant removed each time. Wash beads twice, discarding the supernatant each time.

### Example 8

### Detection of mRNA Transcripts in Vesicles

RNA from the bead-bound vesicles of Example 7 was isolated using the Qiagen miRneasy™ kit, (Cat. No. 217061), according to the manufacturer's instructions.

The vesicles are homogenized in QIAzol™ Lysis Reagent (Qiagen Cat. No. 79306). After addition of chloroform, the homogenate is separated into aqueous and organic phases by centrifugation. RNA partitions to the upper, aqueous phase, while DNA partitions to the interphase and proteins to the lower, organic phase or the interphase. The upper, aqueous phase is extracted, and ethanol is added to provide appropriate binding conditions for all RNA molecules from 18 nucleotides (nt) upwards. The sample is then applied to the RNeasy™ Mini spin column, where the total RNA binds to the membrane and phenol and other contaminants are efficiently washed away. High quality RNA is then eluted in RNase-free water.

RNA from the VCAP bead captured vesicles was measured with the Taqman TMPRSS:ERG fusion transcript assay (Kirsten D. Mertz et al. Neoplasia. 2007 Mar. 9(3): 200-206.). RNA from the 22Rv1 bead captured vesicles was measured with the Taqman SPINK1 transcript assay (Scott A. Tomlins et al. Cancer Cell 2008 Jun. 13(6):519-528). The GAPDH transcript (control transcript) was also measured for both sets of vesicle RNA.

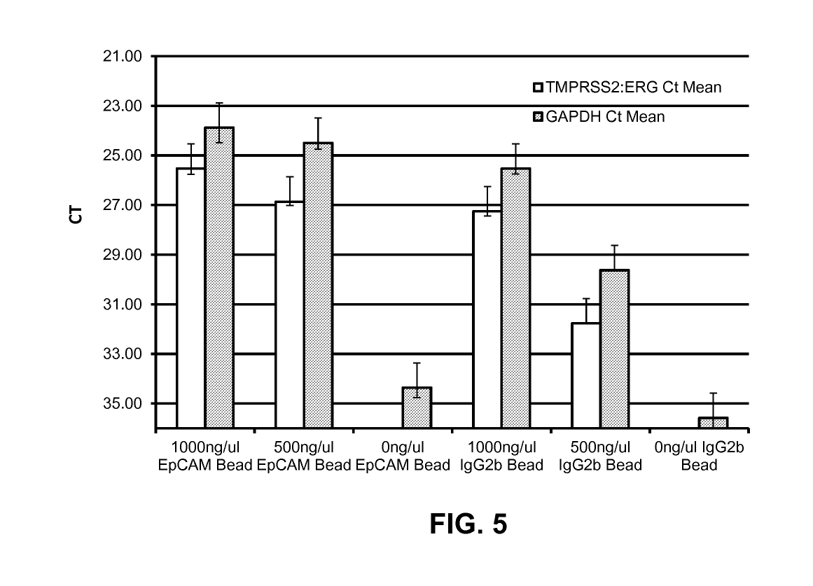

Higher CT values indicate lower transcript expression. One change in cycle threshold (CT) is equivalent to a 2 fold change, 3 CT difference to a 4 fold change, and so forth, which can be calculated with the following: 2̂^{CT1-CT2}. This experiment shows a difference in CT of the expression of the fusion transcript TMPRSS:ERG and the equivalent captured with the IgG2 negative control bead (FIG. 5). The same comparison of the SPINK1 transcript in 22RV1 vesicles showed a CT difference of 6.14 for a fold change of 70.5. Results with GAPDH were similar (not shown).

### Example 9

### Obtaining Serum Samples from Subjects

Blood is collected from subjects (both healthy subjects and subjects with cancer) in EDTA tubes, citrate tubes or in a 10 ml Vacutainer SST plus Blood Collection Tube (BD367985 or BD366643, BD Biosciences). Blood is processed for plasma isolation within 2 h of collection.

Samples are allowed to sit at room temperature for a minimum of 30 min and a max of 2 h. Separation of the clot is accomplished by centrifugation at 1,000-1,300×g at 4° C. for 15-20 min. The serum is removed and dispensed in aliquots of 500 μl into 500 to 750 μl cryotubes. Specimens are stored at −80° C.

At a given sitting, the amount of blood drawn can range from ˜20 to ˜90 ml. Blood from several EDTA tubes is pooled and transferred to RNase/DNase-free 50-ml conical tubes (Greiner), and centrifuged at 1,200×g at room temperature in a Hettich Rotanta 460R benchtop centrifuge for 10 min. Plasma is transferred to a fresh tube, leaving behind a fixed height of 0.5 cm plasma supernatant above the pellet to avoid disturbing the pellet. Plasma is aliquoted, with inversion to mix between each aliquot, and stored at −80° C.

### Example 10

### RNA Isolation from Human Plasma and Serum Samples

Four hundred μl of human plasma or serum is thawed on ice and lysed with an equal volume of 2× Denaturing Solution (Ambion). RNA is isolated using the mirVana PARIS kit following the manufacturer's protocol for liquid samples (Ambion), modified such that samples are extracted twice with an equal volume of acid-phenol chloroform (as supplied by the Ambion kit). RNA is eluted with 105 μl of Ambion elution solution according to the manufacturer's protocol. The average volume of eluate recovered from each column is about 80 μl.

A scaled-up version of the mirVana PARIS (Ambion) protocol is also used: 10 ml of plasma is thawed on ice, two 5-ml aliquots are transferred to 50-ml tubes, diluted with an equal volume of mirVana PARIS 2× Denaturing Solution, mixed thoroughly by vortexing for 30 s and incubated on ice for 5 min. An equal volume (10 ml) of acid/phenol/chloroform (Ambion) is then added to each aliquot. The resulting solutions are vortexed for 1 min and spun for 5 min at 8,000 rpm, 20° C. in a JA17 rotor. The acid/phenol/chloroform extraction is repeated three times. The resulting aqueous volume is mixed thoroughly with 1.25 volumes of 100% molecular-grade ethanol and passed through a mirVana PARIS column in sequential 700-μl aliquots. The column is washed following the manufacturer's protocol, and RNA is eluted in 105 μl of elution buffer (95° C.). A total of 1.5 μl of the eluate is quantified by Nanodrop.

### Example 11

### Measurement of miRNA Levels in RNA from Plasma and Serum Using qRT-PCR

A fixed volume of 1.67 μl of RNA solution from about ˜80 μl -eluate from RNA isolation of a given sample is used as input into the reverse transcription (RT) reaction. For samples in which RNA is isolated from a 400-μl plasma or serum sample, for example, 1.67 μl of RNA solution represents the RNA corresponding to (1.67/80)×400=8.3 μl plasma or serum. For generation of standard curves of chemically synthesized RNA oligonucleotides corresponding to known miRNAs, varying dilutions of each oligonucleotide are made in water such that the final input into the RT reaction has a volume of 1.67 μl. Input RNA is reverse transcribed using the TaqMan miRNA Reverse Transcription Kit and miRNA-specific stem-loop primers (Applied BioSystems) in a small-scale RT reaction comprised of 1.387 μl of H2O, 0.5 μl of 10× Reverse-Transcription Buffer, 0.063 μl of RNase-Inhibitor (20 units/μl), 0.05 μl of 100 mM dNTPs with dTTP, 0.33 μl of Multiscribe Reverse-Transcriptase, and 1.67 μl of input RNA; components other than the input RNA can be prepared as a larger volume master mix, using a Tetrad2 Peltier Thermal Cycler (BioRad) at 16° C. for 30 min, 42° C. for 30 min and 85° C. for 5 min. Real-time PCR is carried out on an Applied BioSystems 7900HT thermocycler at 95° C. for 10 min, followed by 40 cycles of 95° C. for 15 s and 60° C. for 1 min. Data is analyzed with SDS Relative Quantification Software version 2.2.2 (Applied BioSystems.), with the automatic Ct setting for assigning baseline and threshold for Ct determination.

The protocol can also be modified to include a preamplification step, such as for detecting miRNA. A 1.25-μl aliquot of undiluted RT product is combined with 3.75 μl of Preamplification PCR reagents [comprised, per reaction, of 2.5 μl of TaqMan PreAmp Master Mix (2X) and 1.25 μl of 0.2× TaqMan miRNA Assay (diluted in TE)] to generate a 5.0-μl preamplification PCR, which is carried out on a Tetrad2 Peltier Thermal Cycler (BioRad) by heating to 95° C. for 10 min, followed by 14 cycles of 95° C. for 15 s and 60° C. for 4 min. The preamplification PCR product is diluted (by adding 20 μl of H₂O to the 5-μl preamplification reaction product), following which 2.25 μl of the diluted material is introduced into the real-time PCR and carried forward as described.

### Example 12

### Extracting Nucleic Acids from Vesicles

This Example present methods of extracting nucleic acids such as microRNA from vesicles isolated from patient samples as described herein. See, e.g., Example 6. Methods for isolation and concentration of vesicles are presented herein. The methods in this Example can also be used to isolate microRNA directly from patient samples without first isolating vesicles.

Protocol Using Trizol

This protocol uses the QIAzol Lysis Reagent and RNeasy Midi Kit from Qiagen Inc., Valencia Calif. to extract microRNA from concentrated vesicles. The steps of the method comprise:

1. Add 2 μl of RNase A to 50 μl of vesicle concentrate, incubate at 37° C. for 20 min.
2. Add 700 μl of QIAzol Lysis Reagent, vortex 1 minute. Spike samples with 25 fmol/μL of C. elegans microRNA (1 μL) after the addition of QIAzol, making a 75 fmol/μL spike in for each total sample (3 aliquots combined).

### 3. Incubate at 55° C. for 5 min.

4. Add 140 μl chloroform and shake vigorously for 15 sec.

### 5. Cool on ice for 2-3 min.

### 6. Centrifuge @ 12,000×g at 4° C. for 15 min.

7. Transfer aqueous phase (300 μL) to a new tube and add 1.5 volumes of 100% EtOH (i.e., 450 μL).
8. Pipet up to 4 ml of sample into an RNeasy Midi spin column in a 15 ml collection tube (combining lysis from 3 50 μl of concentrate)
9. Spin at 2700×g for 5 min at room temperature.
10. Discard flowthrough from the spin.
11. Add 1 ml of Buffer RWT to column and centrifuge at 2700×g for 5 min at room temperature. Do not use Buffer RW1 supplied in the Midi kit. Buffer RW1 can wash away miRNA. Buffer RWT is supplied in the Mini kit from Qiagen Inc.
12. Discard flowthrough.
13. Add 1 ml of Buffer RPE onto the column and centrifuge at 2700×g for 2 min at room temperature.
14. Repeat steps 12 and 13.
16. Place column into a new 15 ml collection tube and add 150 μl Elution Buffer. Incubate at room temperature for 3 min.
17. Centrifuge at 2700×g for 3 min at room temperature.
18. Vortex the sample and transfer to 1.7 mL tube. Store the extracted sample at −80° C.

Modified Trizol Protocol

1. Add Epicentre RNase A to final concentration of 229 μg/ml (Epicentre®, an Illumina® company, Madison, Wis.). (For example, to 150 ul of concentrate, add 450 μl PBS and 28.8 μl Epicentre Rnase A [5 μg/μl].) Vortex briefly. Incubate for 20 min at 37° C. Aliquot “babies” in increments of 100 μl using reverse pipetting.
2. Set temperature on centrifuge to 4° C.
3. Add 750 μl of Trizol LS to each 100 μl sample and immediately vortex.
5. Incubate on benchtop at room temperature (RT) for 5 mins.
6. Vortex all samples for 30 min. at 1400 rpm at RT in the MixMate. While vortexing, add BCP phase separation agent to the plate.
7. Briefly centrifuge tubes. Transfer the sample to the collection microtube rack.
8. Add 150 μl BCP to the samples in the plate. Cap the plate and shake vigorously for 15 sec.

### 9. Incubate at RT for 3 min.

10. Centrifuge at 6,000×g at 4° C. for 15 min. Reset centrifuge temperature to 24° C. (RT).
11. Add 500 μl 100% EtOH to the appropriate wells of a new S-block. Transfer 200 μl aqueous phase to new S-block, mix the aqueous/EtOH by pipetting 10×.
12. Briefly centrifuge.
13. Place an RNeasy 96 (Qiagen, Inc., Valencia, Calif.) plate on top of a new S-block. Pipette the aqueous/EtOH sample mixture into the wells of the RNeasy 96 plate. Seal the RNeasy 96 plate with AirPore tape.
14. Spin at 6000 rpm (5600×g) for 4 min at RT. Avoid temps below 24° C.
15. Empty the S-block by discarding the flowthrough and remove the AirPore tape.
14. Add 700 μl of Buffer RWT to the plate, seal with AirPore tape, and centrifuge at 6,000 rpm for 4 min at RT. Empty the S-block and remove the AirPore tape.
15. Add 500 μl of Buffer RPE to the plate, seal with AirPore tape, and centrifuge at 6,000 rpm for 4 min at RT. Empty the S-block and remove the AirPore tape.
16. Add another 500 μl of Buffer RPE to the plate, seal with AirPore tape, and centrifuge at 6,000 rpm for 10 min at RT. Empty the S-block and remove the AirPore tape.
17. Place the Rneasy 96 plate on top of a clean elution microtube rack. Pipet 30 μl of RNase-free water onto the columns of the Rneasy 96 plate. Seal with AirPore tape.
18. Allow water to sit on column for 5 min.
19. Centrifuge column for 4 min at 6,000 rpm to elute RNA. Cap the microtubes with elution microtube caps. Pool babies together.

### 20. Store @−80° C.

Protocol Using MagMax

This protocol uses the MagMAX™ RNA Isolation Kit from Applied Biosystems/Ambion, Austin, Tex. to extract microRNA from concentrated vesicles. The steps of the method comprise:

1. Add 700 ml of QIAzol Lysis Reagent and vortex 1 minute.
2. Incubate on benchtop at room temperature for 5 min.
3. Add 140 μl chloroform and shake vigorously for 15 sec.
4. Incubate on benchtop for 2-3 min.

### 5. Centrifuge at 12,000×g at 4° C. for 15 min.

6. Transfer aqueous phase to a deep well plate and add 1.25 volumes of 100% Isopropanol.
7. Shake MagMAX™ binding beads well. Pipet 10 μl of RNA binding beads into each well.
8. Gather two elution plates and two additional deep well plates.
9. Label one elution plate “Elution” and the other “Tip Comb.”
10. Label one deep well as “1st Wash 2” and the other as “2nd Wash 2.”
11. Fill both Wash 2 deep well plates with 150 μl of Wash 2, being sure to add ethanol to wash beforehand. Fill in the same number of wells as there are samples.
12. Select the appropriate collection program on the MagMax Particle Processor.
13. Press start and load each appropriate plate.
14. Transfer samples to microcentrifuge tubes.
15. Vortex and store at −80° C. Residual beads will be seen in sample.

Protocol Using miRNeasy 96 Kit

This modified protocol purifies total RNA 18 to 200 nucleotides in length from vesicles in plasma. An initial phenol:chloroform (BCP) extraction followed by ethanol precipitation is performed prior to washing the samples using the column-based miRNeasy 96 Kit (Qiagen, P/N 217061).

Materials
Proteinase K [50 ug/ul] (Epicentre P/N MPRK092) (optional)
RNase A [5 ug/ul] (Epicentre P/N MRNA092) (optional)
HyClone 1×PBS
Trizol LS
BCP
100% Ethanol
Buffer RWT (Provided in miRNeasy kit (Qiagen))
Buffer RPE (Provided in miRNeasy kit (Qiagen))
Nuclease-free Water (Provided in miRNeasy kit (Qiagen))

Equipment
Heat block set to 55° C. and 37° C.
Vortex
MixMate vortex (holds 24-1.5 ml tubes, not refrigerated, 1400 rpm)
Refrigerated centrifuge with deep-well plate rotor (4° C.-24° C., 6,000 rcf)
Multi-channel pipets (200 μl, 1000 μl)
Single-channel pipets (20 μl, 200 μl, 1000 μl)

Consumables
1.5 mL Seal-rite RNase-free tubes
miRNeasy kit (includes plate-formatted columns, S-block, collection plate)
RNase- and DNase-free barrier tips (20 μl, 200 μl, 1000 μl, 1000 μl extended length)
AirPore Tape (Provided in miRNeasy kit (Qiagen))
Collection Microtube rack and caps (Provided in miRNeasy kit (Qiagen))
Elution Microtube rack and caps (Provided in miRNeasy kit (Qiagen))
RNeasy 96 plate (Provided in miRNeasy kit (Qiagen))
S-block (Provided in miRNeasy kit (Qiagen))
Deep-well, U-bottom plates for flow-thru waste
Clear plate seals
RNase- and DNase-free PCR plate

Methods

Vesicles are first isolated from biological samples as described herein. See, e.g., Example 6, Example 40.

Proteinase K and RNase A Treatment (Optional step to remove protein-bound miRs such as Ago2-bound miRs):
Dilute concentrated plasma with 3× sample volume of 1×PBS.
   For 300 μl concentrated plasma, add 900 μl of 1×PBS.
Add Proteinase K to a final concentration of 833 ug/ml.
   Add 20 μl of Proteinase K [50 ug/ul] to 1200 μl of sample in PBS.
Invert to mix.
Incubate at 55° C. for 60 minutes.
Add RNase A to a final concentration of 229 ug/ml.
   Add 4.8 μl of RNase A [5 ug/ul] to 1200 μl of sample in PBS.
Invert to mix.
Incubate at 37° C. for 20 minutes.

Main Protocol:
1. Prepare 100 μl aliquots of 1:4 sample:PBS (Do not dilute samples further if Proteinase K and RNase A treatments were performed above).
   For 300 μl concentrated plasma, add 900 μl of 1×PBS.
2. Add 750 μl of Trizol LS to each 100 μl aliquot and immediately vortex at high speed for 5 seconds.
3. Incubate samples at room temperature for 5 minutes.
4. Vortex all samples at 1400 rpm for 30 minutes at room temperature.
5. Briefly centrifuge the samples and transfer them from the tubes to the Collection Microtube rack (provided in miRNeasy kit).
6. Carefully add 150 μl of BCP to the samples in the Collection Microtube rack.
7. Securely cap the samples and shake vigorously for 15 seconds.
   Incubate the samples for 3 minutes at room temperature.
8. Centrifuge the samples at 6,000 rcf for 15 minutes at 4° C.
   Reset the centrifuge temperature to 24° C. or room temperature.
   Every subsequent centrifugation steps will be at room temperature.
9. Add 1 ml of 100% EtOH to the wells of a new S-block (provided in miRNeasy kit).
10. Carefully transfer 400 μl (2×200 μl) of sample aqueous phase to the EtOH in the S-block and mix by pipetting up and down 5 times.
   Do not pipet the interphase.
   Adjust the volumes of ethanol and aqueous phase if necessary and maintain a 2.5× volume of ethanol:sample ratio.
11. Cover the S-block with a plate seal and briefly centrifuge.
12. Retrieve a new S-block or deep-well, U-bottom plate (for flow-thru waste) and place a new RNeasy 96 plate on top of it.
13. Transfer the sample in EtOH (˜1400 μl) into the corresponding wells of the RNeasy 96 plate and seal with AirPore tape (provided in miRNeasy kit).
   Centrifuge the RNeasy 96 plate on top of the waste plate at 6000 rpm for 4 minutes at room temperature.
   Replace the waste plate with a new waste plate to prevent well-to-well contamination
   Remove the AirPore tape.
14. Add 700 μl of prepared Buffer RWT (provided in miRNeasy kit) to the RNeasy 96 plate.
   Centrifuge the RNeasy 96 plate on top of the waste plate at 6000 rpm for 4 minutes at room temperature.
   Replace the waste plate with a new waste plate to prevent well-to-well contamination.
   Remove the AirPore tape.
15. Add 500 μl of Buffer prepared RPE (provided in miRNeasy kit) to the RNeasy 96 plate.
   Centrifuge the RNeasy 96 plate on top of the waste plate at 6000 rpm for 4 minutes at room temperature.
   Replace the waste plate with a new waste plate to prevent well-to-well contamination.
   Remove the AirPore tape.
16. Wash the samples again with 500 μl of prepared Buffer RPE to the RNeasy 96 plate.
   Centrifuge the RNeasy 96 plate on top of the waste plate at 6000 rpm for 10 minutes at room temperature.
   Remove the AirPore tape.
17. Place the RNeasy 96 plate on top of a clean Elution Microtube rack (provided in miRNeasy kit) or a new RNase-free PCR plate.
18. Pipet 30 μl of RNase-free water onto the center of the RNeasy 96 plate columns and seal with AirPore tape.
   Allow the water to sit on the column for 5 minutes at room temperature.
   Centrifuge the RNeasy 96 plate on top of the elution plate at 6000 rpm for 4 minutes at room temperature to elute the RNA.
19. Combine RNA extractions from the same initial sample and seal the microtubes or elution plate.

Store RNA samples at −80° C.

### Example 13

### MicroRNA Arrays

MicroRNA levels in a sample can be analyzed using an array format, including both high density and low density arrays. Array analysis can be used to discover differentially expressed in a desired setting, e.g., by analyzing the expression of a plurality of miRs in two samples and performing a statistical analysis to determine which ones are differentially expressed between the samples and can therefore be used in a biosignature. The arrays can also be used to identify a presence or level of one or more microRNAs in a single sample in order to characterize a phenotype by identifying a biosignature in the sample. This Example describes commercially available systems that are used to carry out the methods of the invention.

TaqMan Low Density Array

TaqMan Low Density Array (TLDA) miRNA cards are used to compare expression of miRNA in various sample groups as desired. The miRNA are collected and analyzed using the TaqMan® MicroRNA Assays and Arrays systems from Applied Biosystems, Foster City, Calif. Applied Biosystems TaqMan® Human MicroRNA Arrays are used according to the Megaplex™ Pools Quick Reference Card protocol supplied by the manufacturer.

Exiqon mIRCURY LNA microRNA

The Exiqon miRCURY LNA™ Universal RT microRNA PCR Human Panels I and II (Exiqon, Inc, Woburn, Mass.) are used to compare expression of miRNA in various sample groups as desired. The Exiqon 384 well panels include 750 miRs. Samples are normalized to control primers towards synthetic RNA spike-in from Universal cDNA synthesis kit (UniSp6 CP). Results were normalized to inter-plate calibrator probes.

With either system, quality control standards are implemented. Normalized values for each probe across three data sets for each indication are averaged. Probes with an average CV % higher than 20% are not used for analysis. Results are subjected to a paired t-test to find differentially expressed miRs between two sample groups. P-values are corrected with a Benjamini and Hochberg false-discovery rate test. Results are analyzed using using GeneSpring software (Agilent Technologies, Inc., Santa Clara, Calif.).

### Example 14

### MicroRNA Profiles in Vesicles

Vesicles were collected by ultracentrifugation from 22Rv1, LNCaP, Vcap and normal plasma (pooled from 16 donors) as described in Examples 1-3. RNA was extracted using the Exiqon miR isolation kit (Cat. Nos. 300110, 300111). Equals amounts of vesicles (30 μg) were used as determined by BCA assay.

Equal volumes (5 μl) were put into a reverse-transcription reaction for microRNA. The reverse-transcriptase reactions were diluted in 81 μl of nuclease-free water and then 9 μl of this solution was added to each individual miR assay. MiR-629 was found to only be expressed in PCa (prostate cancer) vesicles and was virtually undetectable in normal plasma vesicles. MiR-9 was found to be highly overexpressed (˜704 fold increase over normal as measured by copy number) in all PCa cell lines, and has very low expression in normal plasma vesicles.

### Example 15

### MicroRNA Profiles of Magnetic EpCam-Captured Vesicles

The bead-bound vesicles of Example 7 were placed in QIAzol™ Lysis Reagent (Qiagen Cat. #79306). An aliquot of 125 fmol of c. elegans miR-39 was added. The RNA was isolated using the Qiagen miRneasy™ kit, (Cat. #217061), according to the manufacturer's instructions, and eluted in 30 ul RNAse free water.

10 μl of the purified RNA was placed into a pre-amplification reaction for miR-9, miR-141 and miR-629 using a Veriti 96-well thermocycler. A 1:5 dilution of the pre-amplification solution was used to set up a qRT-PCR reaction for miR9 (ABI 4373285), miR-141 (ABI 4373137) and miR-629 (ABI 4380969) as well as c. elegans miR-39 (ABI 4373455). The results were normalized to the c. elegans results for each sample.

### Example 16

### MicroRNA Profiles of CD9-Captured Vesicles

CD9 coated Dynal beads (Invitrogen, Carlsbad, Calif.) were used instead of EpCam coated beads as in Example 15. Vesicles from prostate cancer patients, LNCaP, or normal purified vesicles were incubated with the CD9 coated beads and the RNA isolated as described in Example 15. The expression of miR-21 and miR-141 was detected by qRT-PCR and the results depicted in FIG. 6.

### Example 17

### Isolation of Vesicles Using a Filtration Module

Six mL of PBS is added to 1 mL of plasma. Optionally, the sample can be treated with a blocking agent such as StabilGuard®, which may improve downstream processing. The sample is then put through a 1.2 micron (μm) Pall syringe filter directly into a 100 kDa MWCO (Millipore, Billerica, Mass.), 7 ml column with a 150 kDa MWCO (Pierce®, Rockford, Ill.), 15 ml column with a 100 kDa MWCO (Millipore, Billerica, Mass.), or 20 ml column with a 150 kDa MWCO (Pierce®, Rockford, Ill.).

The tube is centrifuged for between 60 to 90 minutes until the volume is about 250 μl. The retentate is collected and PBC added to bring the sample up to 300 μl. Fifty μl of the sample is then used for further vesicle analysis, such as further described in the examples below.

### Example 18

### Multiplex Analysis of Vesicles Isolated with Filters

The vesicle samples obtained using methods as described in Example 17 are used in multiplexing assays as described herein. See, e.g., Examples 23-24 below. The capture antibodies are CD9, CD63, CD81, PSMA, PCSA, B7H3, and EpCam. The detection antibodies are for biomarkers CD9, CD81, and CD63 or B7H3 and EpCam.

### Example 19

### Flow Cytometry Analysis of Vesicles

Purified plasma vesicles are assayed using the MoFlo XDP (Beckman Coulter, Fort Collins, Colo., USA) and the median fluorescent intensity analyzed using the Summit 4.3 Software (Beckman Coulter). Vesicles are labeled directly with antibodies, or beads or microspheres (e.g., magnetic, polystyrene, including BD FACS 7-color setup, catalog no. 335775) can be incorporated. Vesicles can be detected with binding agents against the following vesicle antigens: CD9 (Mouse anti-human CD9, MAB1880, R&D Systems, Minneapolis, Minn., USA), PSM (Mouse anti-human PSM, sc-73651, Santa Cruz, Santa Cruz, Calif., USA), PCSA (Mouse anti-human Prostate Cell Surface Antigen, MAB4089, Millipore, Mass., USA), CD63 (Mouse anti-human CD63, 556019, BD Biosciences, San Jose, Calif., USA), CD81 (Mouse anti-human CD81, 555675, BD Biosciences, San Jose, Calif., USA) B7-H3 (Goat anti-human B7-H3, AF1027, R&D Systems, Minneapolis, Minn., USA), EpCAM (Mouse anti-human EpCAM, MAB9601, R&D Systems, Minneapolis, Minn., USA). Vesicles can be detected with fluorescently labeled antibodies against the desired vesicle antigens. For example, FITC, phycoerythrin (PE) and Cy7 are commonly used to label the antibodies.

To capture the antibodies with multiplex microspheres, the microspheres can be obtained from Luminex (Austin, Tex., USA) and conjugated to the desired antibodies using micros using Sulfo-NHS and EDC obtained from Pierce Thermo (Cat. No. 24510 and 22981, respectively, Rockford, Ill., USA).

Purified vesicles (10 ug/ml) are incubated with 5,000 microspheres for one hour at room temperature with shaking. The samples are washed in FACS buffer (0.5% FBS/PBS) for 10 minutes at 1700 rpms. The detection antibodies are incubated at the manufacturer's recommended concentrations for one hour at room temperature with shaking. Following another wash with FACS buffer for 10 minutes at 1700 rpms, the samples are resuspended in 100 ul FACS buffer and run on the FACS machine.

Further when using microspheres to detect vesicles, the labeled vesicles can be sorted according to their detection antibody content into different tubes. For example, using FITC or PE labeled microspheres, a first tube contains the population of microspheres with no detectors, the second tube contains the population with PE detectors, the third tube contains the population with FITC detectors, and the fourth tube contains the population with both PE and FITC detectors. The sorted vesicle populations can be further analyzed, e.g., by examining payload such as mRNA, microRNA or protein content.

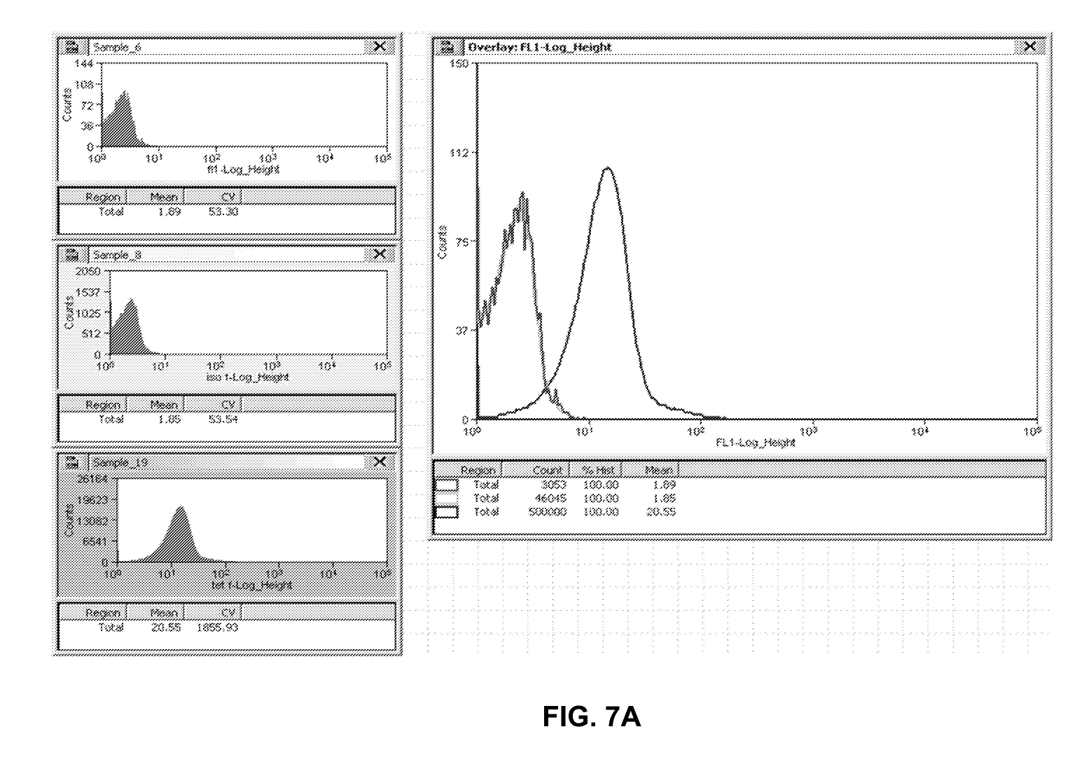

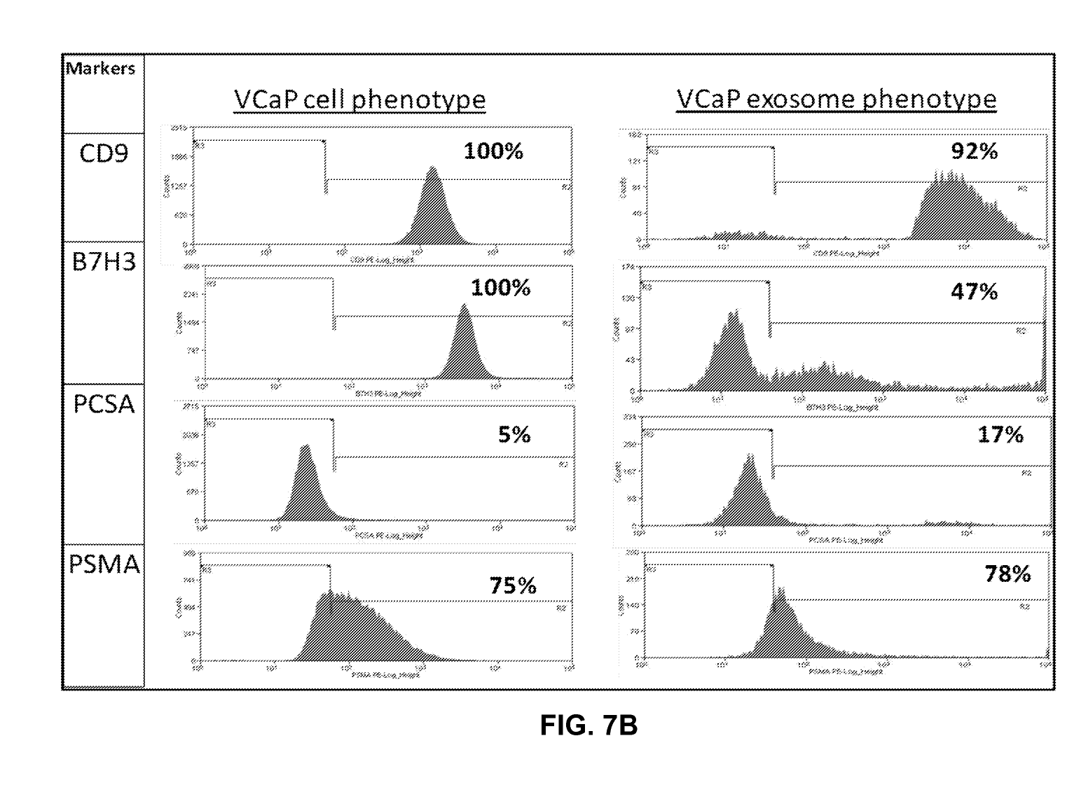

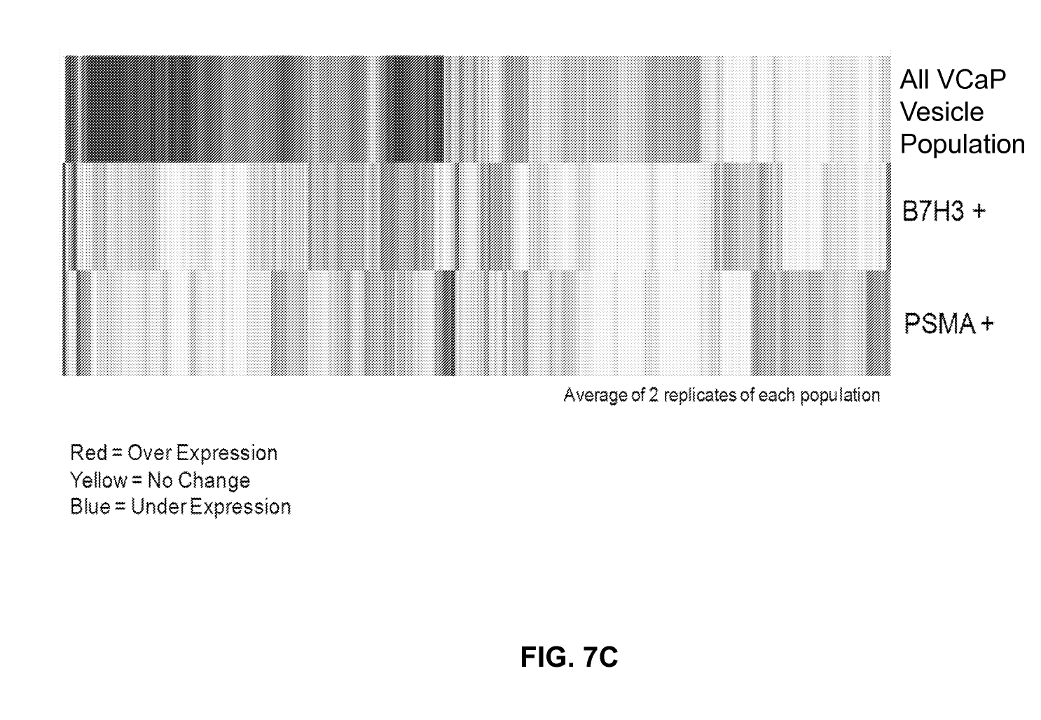

FIG. 7A shows separation and identification of vesicles using the MoFlo XDP. In this set of experiments, there were about 3000 trigger events with just buffer (i.e. particulates about the size of a large vesicle). There were about 46,000 trigger events with unstained vesicles (43,000 vesicles of sufficient size to scatter the laser). There were 500,000 trigger events with stained vesicles. Vesicles were detected using detection agents for tetraspanins CD9, CD63, and CD81 all labeled with FITC. The smaller vesicles can be detected when they are stained with detection agents.

FIG. 7B shows FACS analysis of VCaP cells (left panels) and VCaP exosomes (right panels) for CD9, B7H3, PSMA and PCSA. The analysis demonstrated that both VCaP cells and VCaP-derived exosomes shared similar surface protein markers. Cytofluorometric analysis using flow cytometry revealed that both the VCaP cells and the VCaP-derived vesicles contained CD9, CD63, CD81, PCSA, PSMA and B7-H3 antigens that were accessible to PE-labeled antibodies. Antigens at a lower concentration on the cell surface can be found at a higher concentration on the microvesicle surface (e.g. PCSA).

The microRNA content in flow sorted miRs can differ depending on the marker used to detect the vesicles. VCaP-derived vesicles were sorted using labeled antibodies to B7H3 or PSMA. miR expression patterns in the captured vesicles were determined using Exiqon cards as described herein. FIG. 7C shows that different patterns of expression were obtained in B7H3+ or PSMA+ vesicle populations as compared to overall vesicle population.

Physical isolation by sorting of specific populations of vesicles facilitates additional studies such as microRNA analysis on the partially or wholly purified vesicle populations.

### Example 20

### Antibody Detection of Vesicles

Vesicles in a patient sample are assessed using antibody-coated beads to detect the vesicles in the sample using techniques as described herein. The following general protocol is used:
a. Blood is drawn from a patient at a point of care (e.g., clinic, doctor's office, hospital).
b. The plasma fraction of the blood is used for further analysis.
c. To remove large particles and isolate a vesicle containing fraction, the plasma sample is filtered, e.g., with a 0.8 or 1.2 micron (μm) syringe filter, and then passed through a size exclusion column, e.g., with a 150 kDa molecular weight cut off. A general schematic is shown in FIG. 8A. Filtration may be preferable to ultracentrifugation, as illustrated in FIG. 8B. Without being bound by theory, high-speed centrifugation may remove protein targets weakly anchored in the membrane as opposed to the tetraspanins which are more solidly anchored in the membrane, and may reduce the cell specific targets in the vesicle, which would then not be detected in subsequent analysis of the biosignature of the vesicle.
d. The vesicle fraction is incubated with beads conjugated with a “capture” antibody to a marker of interest. The captured vesicles are then tagged with labeled “detection” antibodies, e.g., phycoerythrin or FITC conjugated antibodies. The beads can be labeled as well.
e. Captured and tagged vesicles in the sample are detected. Fluorescently labeled beads and detection antibodies can be detected as shown in FIG. 8C. Use of the labeled beads and labeled detection antibodies allows assessment of beads with vesicles bound thereto by the capture antibody. Note that the figure is simplified for purposes of illustration. For example, different detectors can be used for each laser.
f. Data is analyzed. A threshold can be set for the median fluorescent intensity (MFI) of a particular capture antibody. A reading for that capture antibody above the threshold can indicate a certain phenotype. As an illustrative example, an MFI above the threshold for a capture antibody directed to a cancer marker can indicate the presense of cancer in the patient sample.

In FIG. 8C, the beads 816 flow through a capillary 811. Use of dual lasers 812 at different wavelengths allows separate detection at detector 813 of both the capture antibody 818 from the fluorescent signal derived from the bead, as well as the median fluorescent intensity (MFI) resulting from the labeled detection antibodies 819. Use of labeled beads conjugated to different capture antibodies of interest, each bead labeled with a different fluor, allows for mulitiplex analysis of different vesicle 817 populations in a single assay as shown. Laser 1 815 allows detection of bead type (i.e., the capture antibody) and Laser 2 814 allows measurement of detector antibodies, which can include general vesicle markers such as tetraspanins including CD9, CD63 and CD81. Use of different populations of beads and lasers allows simultaneous multiplex analysis of many different populations of vesicles in a single assay.

FIG. 8D represents an example of detecting prostate-cancer derived vesicles bound to a substrate using the general protocol in this Example. The microvesicles are captured with capture agents specific to PCSA, PSMA or B7H3 tethered to the substrate (i.e., beads). The so-captured vesicles are labeled with fluorescently labeled detection agents specific to tetraspanins CD9, CD63 and CD81.

The MFI values obtained using the microsphere assay correlate with the levels of the target proteins as determined by alternate methods. Levels of VCap derived vesicles were compared between the microsphere assay, FACS, and BCA protein assay. Analysis of CD9-labeled vesicles demonstrated tight correlation between MFI and number of vesicles as determined by Flow analysis, as shown in FIG. 8E. Analysis using PSMA, PCSA and B7H3 as vesicle markers showed that total protein concentration from VCaP vesicles measured using the BCA protein assay also correlated tightly to the MFI value determined on the microvesicle assay, as shown in FIG. 8F.

The microsphere assay can be used to detect markers in a multiplex format without hinderence in assay performance. For example, we found no competition effect observed by the multiplexing of 6 different capture antibodies (PSMA, PCSA, B7-H3, CD9, CD63, CD81). The MFIs recorded for the multiplexed method were identical to the MFIs recorded for each individual marker when run in a single-plea assay format. Comparison of the distribution of MFI values obtained using the cMV-based assay that used multiplexed antibodies with one that included a single antibody against the biomarker CD81 are shown in FIG. 8G. Frequency is expressed as the normalized number of beads. Singleplex vs multiplex B7H3, CD63, CD9, and EpCam capture antibody comparisons also showed no interference in a multiplex format at two different non-saturating VCaP vesicle concentrations, as shown in FIG. 8H.

### Example 21

### Detection of Prostate Cancer

High quality training set samples were obtained from commercial suppliers. The samples comprised plasma from 42 normal prostate, 42 PCa and 15 BPH patients. The PCa samples included 4 stage III and the remainder state II. The samples were blinded until all laboratory work was completed.

The vesicles from the samples were obtained by filtration to eliminate particles greater than 1.5 microns, followed by column concentration and purification using hollow fiber membrane tubes. The samples were analyzed using a multiplexed bead-based assay system as described above.

Antibodies to the following proteins were analyzed:
a. General Vesicle (MV) markers: CD9, CD81, and CD63
b. Prostate MV markers: PCSA
c. Cancer-Associated MV markers: EpCam and B7H3

Samples were required to pass a quality test as follows: if multiplexed median fluorescence intensity (MFI) PSCA+MFI B7H3+MFI EpCam<200 then sample fails due to lack of signal above background. In the training set, six samples (three normals and three prostate cancers) did not achieve an adequate quality score and were excluded. An upper limit on the MFI was also established as follows: if MFI of EpCam is >6300 then test is over the upper limit score and samples are deemed not cancer (i.e., “negative” for purposes of the test).

The samples were classified according to the result of MFI scores for the six antibodies to the training set proteins, wherein the following conditions must be met for the sample to be classified as PCa positive:
a. Average MFI of General MV markers>1500
b. PCSA MFI>300
c. B7H3 MFI>550
d. EpCam MFI between 550 and 6300

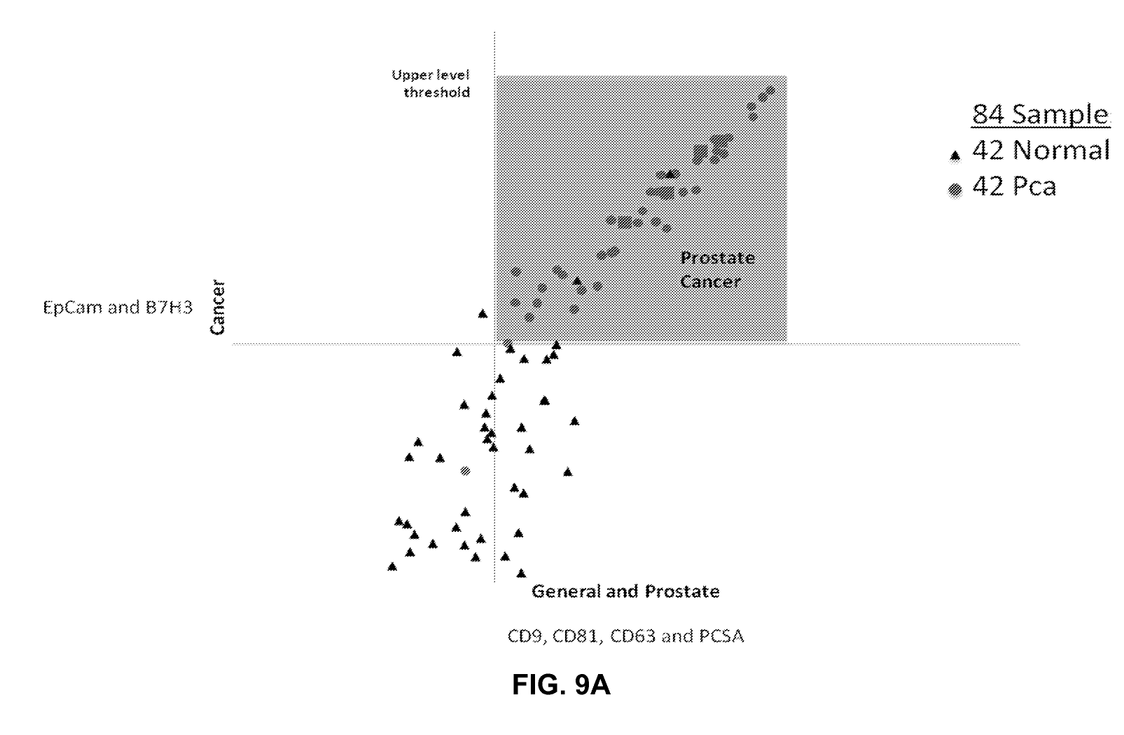

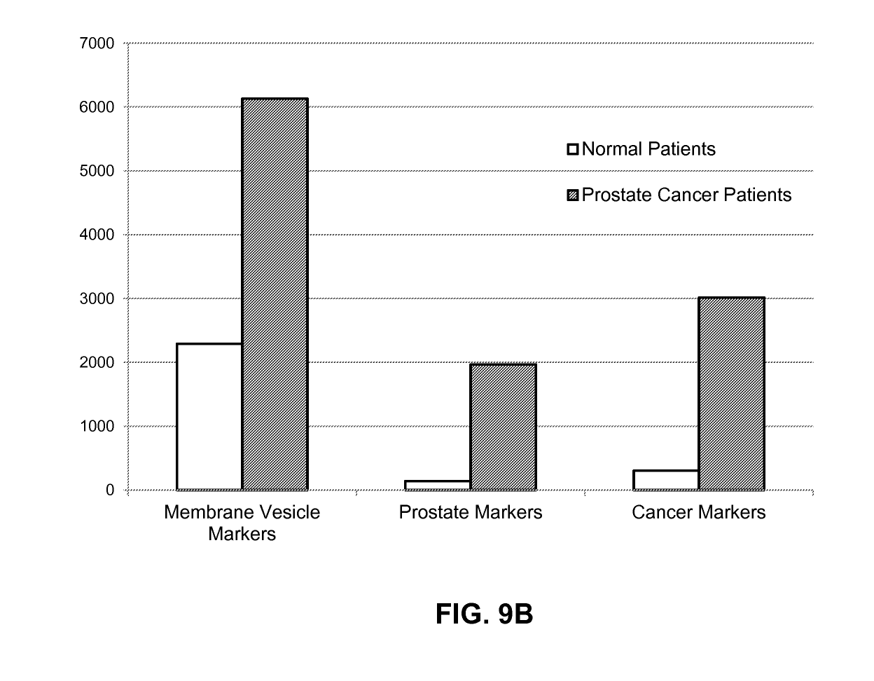

Using the 84 normal and PCa training data samples, the test was found to be 98% sensitive and 95% specific for PCa vs normal samples. See FIG. 9A. The increased MFI of the PCa samples compared to normals is shown in FIG. 9B. Compared to PSA and PCA3 testing, the PCa Test presented in this Example can result in saving ˜220 men without PCa in every 1000 normal men screened from having an unnecessary biopsy.

### Example 22

### Microsphere Vesicle Prostate Cancer Assay Protocol

In this example, the vesicle PCa test is a microsphere based immunoassay for the detection of a set of protein biomarkers present on the vesicles from plasma of patients with prostate cancer. The test employs specific antibodies to the following protein biomarkers: CD9, CD59, CD63, CD81, PSMA, PCSA, B7H3 and EpCAM. After capture of the vesicles by antibody coated microspheres, phycoerythrin-labeled antibodies are used for the detection of vesicle specific biomarkers. Depending on the level of binding of these antibodies to the vesicles from a patient's plasma a determination of the presence or absence of prostate cancer is made.

Vesicles are isolated as described above.

Microspheres

Specific antibodies are conjugated to microspheres (Luminex) after which the microspheres are combined to make a Microsphere Master Mix consisting of L100-C105-01; L100-C115-01; L100-C119-01; L100-C120-01; L100-C122-01; L100-C124-01; L100-C135-01; and L100-C175-01. xMAPO Classification Calibration Microspheres L100-CAL1 (Luminex) are used as instrument calibration reagents for the Luminex LX200 instrument. xMAPO Reporter Calibration Microspheres L100-CAL2 (Luminex) are used as instrument reporter calibration reagents for the Luminex LX200 instrument. xMAPO Classification Control Microspheres L100-CON1 (Luminex) are used as instrument control reagents for the Luminex LX200 instrument. xMAP Reporter Control Microspheres L100-CON2 (Luminex) and are used as reporter control reagents for the Luminex LX200 instrument.

Capture Antibodies

The following antibodies are used to coat Luminex microspheres for use in capturing certain populations of vesicles by binding to their respective protein targets on the vesicles in this Example: a. Mouse anti-human CD9 monoclonal antibody is an IgG2b used to coat microsphere L100-C105 to make *EPCLMACD9-C105; b. Mouse anti-human PSMA monoclonal antibody is an IgG1 used to coat microsphere L100-C115 to make EPCLMAPSMA-C115; c. Mouse anti-human PCSA monoclonal antibody is an IgG1 used to coat microsphere L100-C119 to make EPCLMAPCSA-C119; d. Mouse anti-human CD63monoclonal antibody is an IgG1 used to coat microsphere L100-C120 to make EPCLMACD63-C120; e. Mouse anti-human CD81 monoclonal antibody is an IgG1 used to coat microsphere L100-C124 to make EPCLMACD81-C124; f. Goat anti-human B7-H3 polyclonal antibody is an IgG purified antibody used to coat microsphere L100-C125 to make EPCLGAB7-H3-C125; and g. Mouse anti-human EpCAM monoclonal antibody is an IgG2b purified antibody used to coat microsphere L100-C175 to make EPCLMAEpCAM-C175.

Detection Antibodies

The following phycoerythrin (PE) labeled antibodies are used as detection probes in this assay: a. EPCLMACD81PE: Mouse anti-human CD81 PE labeled antibody is an IgG1 antibody used to detect CD81 on captured vesicles; b. EPCLMACD9PE: Mouse anti-human CD9 PE labeled antibody is an IgG1 antibody used to detect CD9 on captured vesicles; c. EPCLMACD63PE: Mouse anti-human CD63 PE labeled antibody is an IgG1 antibody used to detect CD63 on captured vesicles; d. EPCLMAEpCAMPE: Mouse anti-human EpCAM PE labeled antibody is an IgG1 antibody used to detect EpCAM on captured vesicles; e. EPCLMAPSMAPE: Mouse anti-human PSMA PE labeled antibody is an IgG1 antibody used to detect PSMA on captured vesicles; f. EPCLMACD59PE: Mouse anti-human CD59 PE labeled antibody is an IgG1 antibody used to detect CD59 on captured vesicles; and g. EPCLMAB7-H3PE: Mouse anti-human B7-H3 PE labeled antibody is an IgG1 antibody used to detect B7-H3 on captured vesicles.

Reagent Preparation

Antibody Purification:

The following antibodies in Table 13 are received from vendors and purified and adjusted to the desired working concentrations according to the following protocol.

TABLE 13
Antibodies for PCa Assay

| Antibody | Use |
|---|---|
| EPCLMACD9 | Coating of microspheres for vesicle capture |
| EPCLMACD63 | Coating of microspheres for vesicle capture |
| EPCLMACD81 | Coating of microspheres for vesicle capture |
| EPCLMAPSMA | Coating of microspheres for vesicle capture |
| EPCLGAB7-H3 | Coating of microspheres for vesicle capture |
| EPCLMAEpCAM | Coating of microspheres for vesicle capture |
| EPCLMAPCSA | Coating of microspheres for vesicle capture |
| EPCLMACD81PE | PE coated antibody for vesicle biomarker |
| | detection |
| EPCLMACD9PE | PE coated antibody for vesicle biomarker |
| | detection |
| EPCLMACD63PE | PE coated antibody for vesicle biomarker |
| | detection |
| EPCLMAEpCAMPE | PE coated antibody for vesicle biomarker |
| | detection |
| EPCLMAPSMAPE | PE coated antibody for vesicle biomarker |
| | detection |
| EPCLMACD59PE | PE coated antibody for vesicle biomarker |
| | detection |
| EPCLMAB7-H3PE | PE coated antibody for vesicle biomarker |
| | detection |

Antibody Purification Protocol: Antibodies are purified using Protein G resin from Pierce (Protein G spin kit, prod #89979). Micro-chromatography columns made from filtered P-200 tips are used for purification.

One hundred μl of Protein G resin is loaded with 100 μl buffer from the Pierce kit to each micro column. After waiting a few minutes to allow the resin to settle down, air pressure is applied with a P-200 Pipettman to drain buffer when needed, ensuring the column is not let to dry. The column is equilibrated with 0.6 ml of Binding Buffer (pH 7.4, 100 mM Phosphate Buffer, 150 mM NaCl; (Pierce, Prod #89979). An antibody is applied to the column (<1 mg of antibody is loaded on the column). The column is washed with 1.5 ml of Binding Buffer. Five tubes (1.5 ml micro centrifuge tubes) are prepared and 10 μl of neutralization solution (Pierce, Prod #89979) is applied to each tube. The antibody is eluted with the elution buffer from the kit to each of the five tubes, 100 μl for each tube (for a total of 500 μl). The relative absorbance of each fraction is measured at 280 nm using Nanodrop (Thermo scientific, Nanodrop 1000 spectrophotometer). The fractions with highest OD reading are selected for downstream usage. The samples are dialyzed against 0.25 liters PBS buffer using Pierce Slide-A-Lyzer Dialysis Cassette (Pierce, prod 66333, 3KDa cut off). The buffer is exchanged every 2 hours for minimum three exchanges at 4° C. with continuous stirring. The dialyzed samples are then transferred to 1.5 ml microcentifuge tubes, and can be labeled and stored at 4° C. (short term) or −20° C. (long term).

Microsphere Working Mix Assembly: A microsphere working mix MWM101 includes the first four rows of antibody, microsphere and coated microsphere of Table 14.

TABLE 14
Antibody-Microsphere Combinations

| Antibody | Microsphere | Coated Microsphere |
|---|---|---|
| EPCLMACD9 | L100-C105 | EPCLMACD9-C105 |
| EPCLMACD63 | L100-C120 | EPCLMACD63-C120 |
| EPCLMACD81 | L100-C124 | EPCLMACD81-C124 |
| EPCLMAPSMA | L100-C115 | EPCLMAPSMA-C115 |
| EPCLGAB7-H3 | L100-C125 | EPCLGAB7-H3-C125 |
| bEPCLMAEpCAM | L100-C175 | EPCLMAEpCAM-C175 |
| EPCLMAPCSA | L100-C119 | EPCLMAPCSA-C119 |

Microspheres are coated with their respective antibodies as listed above according to the following protocol.

Protocol for Two-Step Carbodiimide Coupling of Protein to Carboxylated Microspheres: The microspheres should be protected from prolonged exposure to light throughout this procedure. The stock uncoupled microspheres are resuspended according to the instructions described in the Product Information Sheet provided with the microspheres (xMAP technologies, MicroPlex™ Microspheres). Five×106 of the stock microspheres are transferred to a USA Scientific 1.5 ml microcentrifuge tube. The stock microspheres are pelleted by microcentrifugation at ≧8000×g for 1-2 minutes at room temperature. The supernatant is removed and the pelleted microspheres are resuspended in 100 μl of dH2O by vortex and sonication for approximately 20 seconds. The microspheres are pelleted by microcentrifugation at ≧8000×g for 1-2 minutes at room temperature. The supernatant is removed and the washed microspheres are resuspended in 80 μl of 100 mM Monobasic Sodium Phosphate, pH 6.2 by vortex and sonication (Branson 1510, Branson UL Trasonics Corp.) for approximately 20 seconds. Ten μl of 50 mg/ml Sulfo-NHS (Thermo Scientific, Cat#24500) (diluted in dH2O) is added to the microspheres and is mixed gently by vortex. Ten μl of 50 mg/ml EDC (Thermo Scientific, Cat#25952-53-8) (diluted in dH2O) is added to the microspheres and gently mixed by vortexing. The microspheres are incubated for 20 minutes at room temperature with gentle mixing by vortex at 10 minute intervals. The activated microspheres are pelleted by microcentrifugation at ≧8000×g for 1-2 minutes at room temperature. The supernatant is removed and the microspheres are resuspended in 250 μl of 50 mM MES, pH 5.0 (MES, Sigma, Cat# M2933) by vortex and sonication for approximately 20 seconds. (Only PBS-1% BSA+Azide (PBS-BN)((Sigma (P3688-10PAK+0.05% NaAzide (S8032))) should be used as assay buffer as well as wash buffer.). The microspheres are then pelleted by microcentrifugation at ≧8000×g for 1-2 minutes at room temperature.

The supernatant is removed and the microspheres are resuspended in 250 μl of 50 mM MES, pH 5.0 (MES, Sigma, Cat# M2933) by vortex and sonication for approximately 20 seconds. (Only PBS-1% BSA+Azide (PBS-BN) ((Sigma (P3688-10PAK+0.05% NaAzide (S8032))) should be used as assay buffer as well as wash buffer.). The microspheres are then pelleted by microcentrifugation at ≧8000×g for 1-2 minutes at room temperature, thus completing two washes with 50 mM MES, pH 5.0.

The supernatant is removed and the activated and washed microspheres are resuspended in 100 μl of 50 mM MES, pH 5.0 by vortex and sonication for approximately 20 seconds. Protien in the amount of 125, 25, 5 or 1 μg is added to the resuspended microspheres. (Note: Titration in the 1 to 125 μg range can be performed to determine the optimal amount of protein per specific coupling reaction.). The total volume is brought up to 500 μl with 50 mM MES, pH 5.0. The coupling reaction is mixed by vortex and is incubated for 2 hours with mixing (by rotating on Labquake rotator, Barnstead) at room temperature. The coupled microspheres are pelleted by microcentrifugation at ≧8000×g for 1-2 minutes at room temperature. The supernatant is removed and the pelleted microspheres are resuspended in 500 μL of PBS-TBN by vortex and sonication for approximately 20 seconds. (Concentrations can be optimized for specific reagents, assay conditions, level of multiplexing, etc. in use.).

The microspheres are incubated for 30 minutes with mixing (by rotating on Labquake rotator, Barnstead) at room temperature. The coupled microspheres are pelleted by microcentrifugation at ≧8000×g for 1-2 minutes at room temperature. The supernatant is removed and the microspheres are resuspended in 1 ml of PBS-TBN by vortex and sonication for approximately 20 seconds. (Each time there is the addition of samples, detector antibody or SA-PE the plate is covered with a sealer and light blocker (such as aluminum foil), placed on the orbital shaker and set to 900 for 15-30 seconds to re-suspend the beads. Following that the speed should be set to 550 for the duration of the incubation.).

The microspheres are pelleted by microcentrifugation at ≧8000×g for 1-2 minutes. The supernatant is removed and the microspheres are resuspended in 1 ml of PBS-TBN by vortex and sonication for approximately 20 seconds. The microspheres are pelleted by microcentrifugation at ≧8000×g for 1-2 minutes (resulting in a total of two washes with 1 ml PBS-TBN).

Protocol for Microsphere Assay:

The preparation for multiple phycoerythrin detector antibodies is used as described in Example 4. One hundred is analyzed on the Luminex analyzer (Luminex 200, xMAP technologies) according to the system manual (High PMT setting).

Decision Tree:

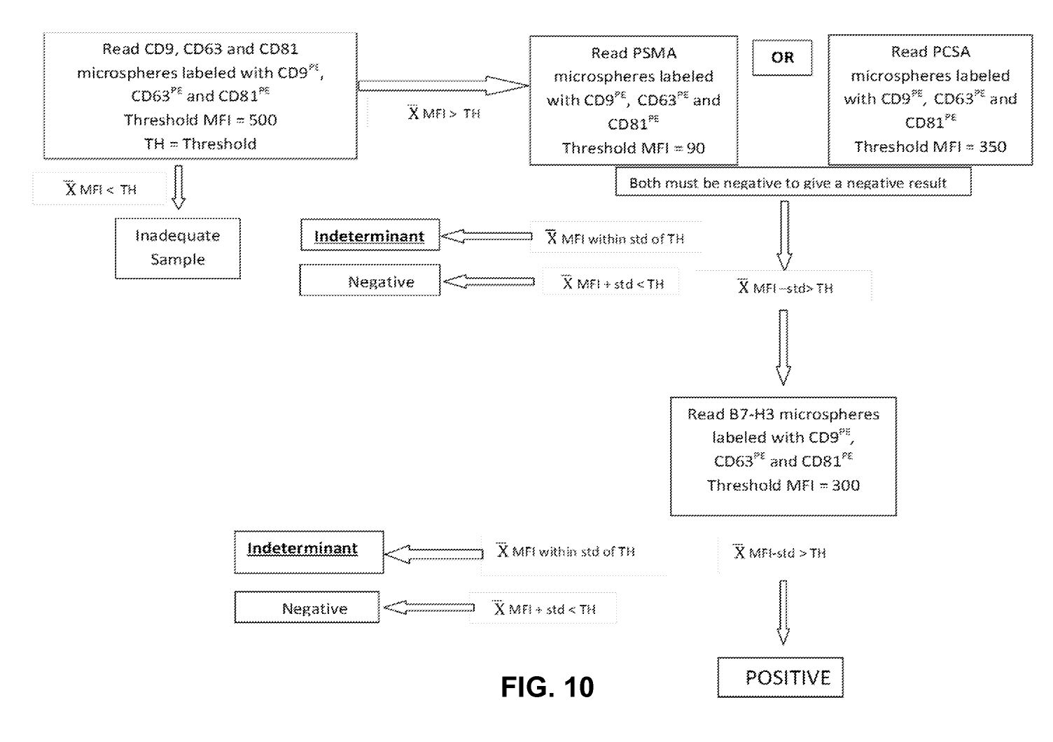

A decision tree as in FIG. 10 is used to assess the results from the microsphere assay to determine if a subject has cancer. Threshold limits on the MFI is established and samples classified according to the result of MFI scores for the antibodies, to determine whether a sample has sufficient signal to perform analysis (e.g., is a valid sample for analysis or an invalid sample for further analysis, in which case a second patient sample may be obtained) and whether the sample is PCa positive. FIG. 10 shows a decision tree using the MFI obtained with PCSA, PSMA, B7-H3, CD9, CD81 and CD63. A sample is classified as indeterminate if the MFI is within the standard deviation of the predetermined threshold (TH). In this case, a second patient sample can be obtained. For validation, the sample must have sufficient signal when capturing vesicles with the individual tetraspanins and labeling with all tetraspanins. A sample that passes validation is called positive if either of the prostate-specific markers (PSMA or PCSA) is considered positive, and the cancer marker (B7-H3) is also considered positive.

Results: See Example 23.

### Example 23

### Microsphere Vesicle PCa Assay Performance

In this example, the vesicle PCa test is a microsphere based immunoassay for the detection of a set of protein biomarkers present on the vesicles from plasma of patients with prostate cancer. The test is performed similarly to that of Example 22 with modifications indicated below.

The test uses a multiplexed immunoassay designed to detect circulating microvesicles. The test uses PCSA, PSMA and B7H3 to capture the microvesicles present in patient samples such as plasma and uses CD9, CD81, and CD63 to detect the captured microvesicles. The output of this assay is the median fluorescent intensity (MFI) that results from the antibody capture and fluorescently labeled antibody detection of microvesicles that contain both the individual capture protein and the detector proteins on the microvesicle. A sample is “POSITIVE” by this test if the MFI levels of PSMA or PCSA, and B7H3 protein-containing microvesicles are above the empirically determined threshold. A method for determining the threshold is presented in Example 33 of International Patent Application Serial No. PCT/US2011/031479, entitled “Circulating Biomarkers for Disease” and filed Apr. 6, 2011, which application is incorporated by reference in its entirety herein. A sample is determined to be “NEGATIVE” if any one of these two microvesicle capture categories exhibit an MFI level that is below the empirically determined threshold. Alternatively, a result of “INDETERMINATE” will be reported if the sample MFI fails to clearly produce a positive or negative result due to MFI values not meeting certain thresholds or the replicate data showed too much statistical variation. A “NON-EVALUABLE” interpretation for this test indicates that this patient sample contained inadequate microvesicle quality for analysis. See Example 33 of International Patent Application Serial No. PCT/US2011/031479 for a method to determine the empirically derived threshold values.

The test employs specific antibodies to the following protein biomarkers: CD9, CD59, CD63, CD81, PSMA, PCSA, and B7H3 as in Example 22. Decision rules are set to determine if a sample is called positive, negative or indeterminate, as outlined in Table 15. See also Example 22. For a sample to be called positive the replicates must exceed all four of the MFI cutoffs determined for the tetraspanin markers (CD9, CD63, CD81), prostate markers (PSMA or PCSA), and B7H3. Samples are called indeterminate if both of the three replicates from PSMA and PCSA or any of the three replicates from B7H3 antibodies span the cutoff MFI value. Samples are called negative if there is at least one of the tetraspanin markers (CD9, CD63, and CD81), prostate markers (PSMA or PCSA), B7H3 that fall below the MFI cutoffs.

TABLE 15
MFI Parameter for Each Capture Antibody

| Tetraspanin Markers | Prostate Markers | | Result |
|---|---|---|---|
| (CD9, CD63, CD81) | (PSMA, PCSA) | B7H3 | Determination |
| Average of all | All replicates from | All replicates from | If all 3 are true, |
| replicates from the | either of the two | B7H3 have a MFI | then the sample is |
| three tetraspanins have | prostate markers have | >300 | called Positive |
| a MFI >500 | a MFI >350 for PCSA | | |
| | and >90 for PSMA | | |
| | Both replicate sets | Any replicates | If either are true, |
| | from either prostate | from B7H3 have | then the sample is |
| | marker have values | values both above | called |
| | both above and below | and below a MFI = | indeterminate |
| | a MFI = 350 for PCSA | 300 | |
| | and = 90 for PSMA | | |
| All replicates from the | All replicates from | All replicates from | If any of the 3 are |
| three tetraspanins have | either of the two | B7H3 have a MFI | true, then the |
| a MFI <500 | prostate markers have | <300 | sample is called |
| | a MFI <350 for PCSA | | Negative, given the |
| | and <90 for PSMA | | sample doesn't |
| | | | qualify as |
| | | | indeterminate |

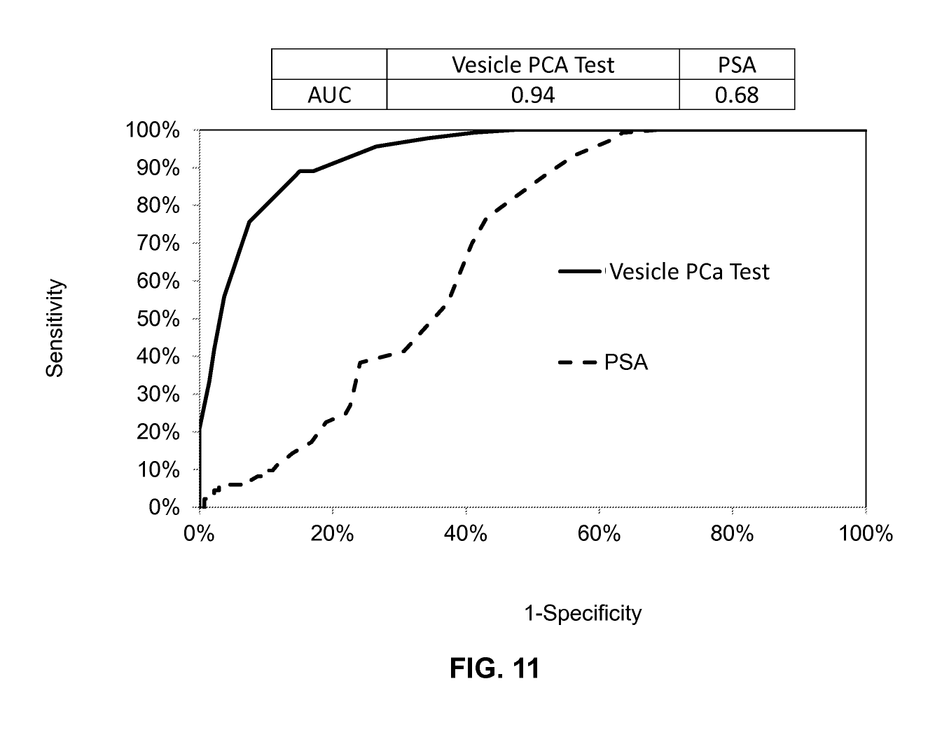

The vesicle PCa test was compared to elevated PSA on a cohort of 296 patients with or without PCa as confirmed by biopsy. An ROC curve of the results is shown in FIG. 11. As shown, the area under the curve (AUC) for the vesicle PCa test was 0.94 whereas the AUC for elevated PSA on the same samples was only 0.68. The PCa samples were likely found due to a high PSA value. Thus this population is skewed in favor of PSA, accounting for the higher AUC than is observed in a true clinical setting.

The vesicle PCa test was further performed on a cohort of 933 patient plasma samples. Results are summarized in Table 16:

TABLE 16
Performance of vesicle PCa test on 933 patient cohort

| | |
|---|---|
| True Positive | 409 |
| True Negative | 307 |
| False Positive | 50 |
| False Negative | 72 |
| Non-evaluable | 63 |
| Indeterminate | 32 |
| Total | 933 |
| Sensitivity | 85% |
| Specificity | 86% |
| Accuracy | 85% |
| Non-evaluable Rate | 8% |
| Indeterminate Rate | 5% |

As shown in Table 16, the vesicle PCa test achieved an 85% sensitivity level at a 86% specificity level, for an accuracy of 85%. In contrast, PSA at a sensitivity of 85% had a specificity of about 55%, and PSA at a specificity of 86% had a sensitivity of about 5%. FIG. 11. About 12% of the 933 samples were non-evaluable or indeterminate. Samples from the patients could be recollected and re-evaluated. The vesicle PCa test had an AUC of 0.92 for the 933 samples.

### Example 24

### Vesicle Protein Array to Detect Prostate Cancer

In this example, the vesicle PCa test is performed using a protein array, more specifically an antibody array, for the detection of a set of protein biomarkers present on the vesicles from plasma of patients with prostate cancer. The array comprises capture antibodies specific to the following protein biomarkers: CD9, CD59, CD63, CD81. Vesicles are isolated as described above, e.g., in Example 20. After filtration and isolation of the vesicles from plasma of men at risk for PCa, such as those over the age of 50, the plasma samples are incubated with an array harboring the various capture antibodies. Depending on the level of binding of fluorescently labeled detection antibodies to PSMA, PCSA, B7H3 and EpCAM that bind to the vesicles from a patient's plasma that hybridize to the array, a determination of the presence or absence of prostate cancer is made.

In a second array format, the vesicles are isolated from plasma and hybridized to an array containing CD9, CD59, CD63, CD81, PSMA, PCSA, B7H3 and EpCam. The captured vesicles are tagged with non-specific vesicle antibodies labeled with Cy3 and/or Cy5. The fluorescence is detected. Depending on the pattern of binding, a determination of the presence or absence of prostate cancer is made.

### Example 25

### Distinguishing BPH and PCa Using miRs

RNA from the plasma derived vesicles of nine normal male individuals and nine individuals with stage 3 prostate cancers were analyzed on the Exiqon mIRCURY LNA microRNA PCR system panel. The Exiqon 384 well panels measure 750 miRs. Samples were normalized to control primers towards synthetic RNA spike-in from Universal cDNA synthesis kit (UniSp6 CP). Normalized values for each probe across three data sets for each indication (BPH or PCa) were averaged. Probes with an average CV % higher than 20% were not used for analysis.

Analysis of the results revealed several microRNAs that were 2 fold or more over-expressed in BPH samples compared to Stage 3 prostate cancer samples. These miRs include: hsa-miR-329, hsa-miR-30a, hsa-miR-335, hsa-miR-152, hsa-miR-151-5p, hsa-miR-200a and hsa-miR-145, as shown in Table 17:

TABLE 17
miRs overexpressed in BPH vs PCa

| Overexpressed in BPH v PCa | Fold Change |
|---|---|
| hsa-miR-329 | 12.32 |
| hsa-miR-30a | 6.16 |
| hsa-miR-335 | 6.00 |
| hsa-miR-152 | 4.73 |
| hsa-miR-151-5p | 3.16 |
| hsa-miR-200a | 3.16 |
| hsa-miR-145 | 2.35 |

### Example 26

### miR-145 in Controls and PCa Samples

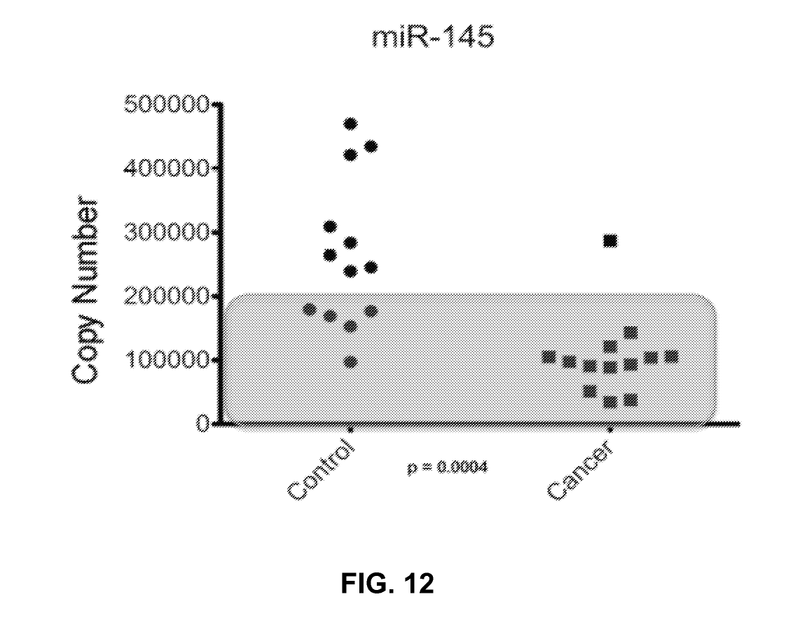

FIG. 12 illustrates a comparison of miR-145 in control and prostate cancer samples. RNA was collected as in Example 12. The controls include Caucasians>75 years old and African Americans>65 years old with PSA<4 ng/ml and a benign digital rectal exam (DRE). As seen in the figure, miR-145 was under expressed in PCa samples. miR-145 is useful for identifying those with early/indolent PCa vs those with benign prostate changes (e.g., BPH).

### Example 27

### miRs to Enhance Vesicle Diagnostic Assay Performance

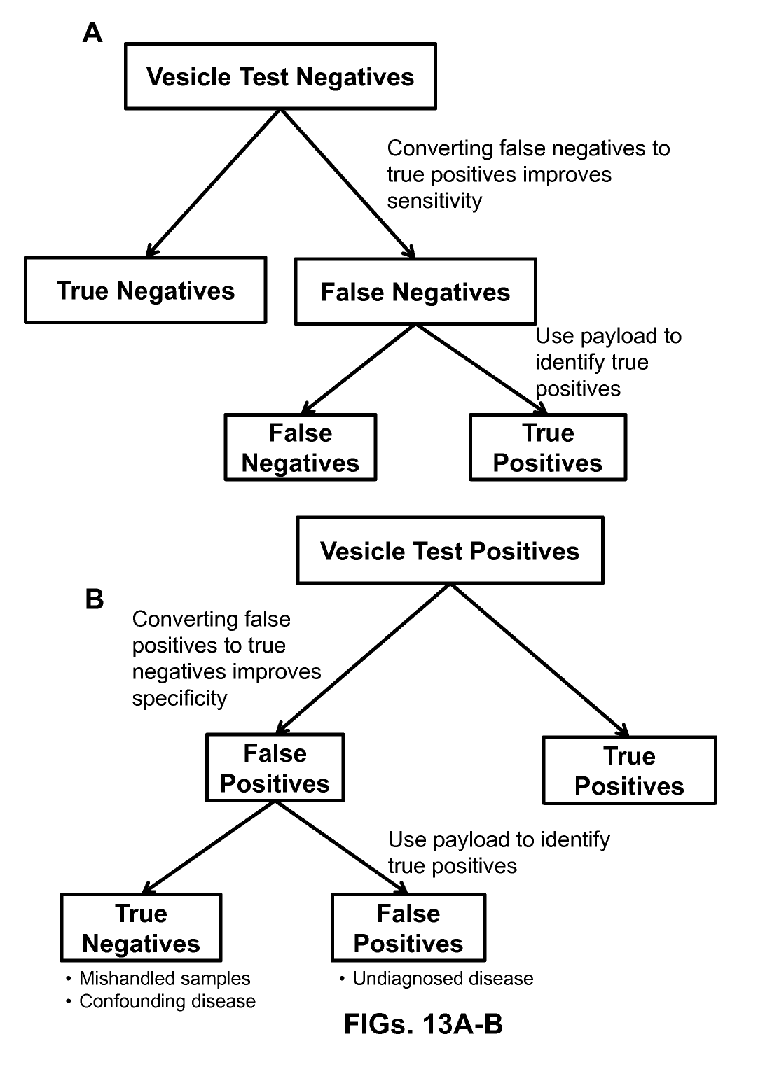

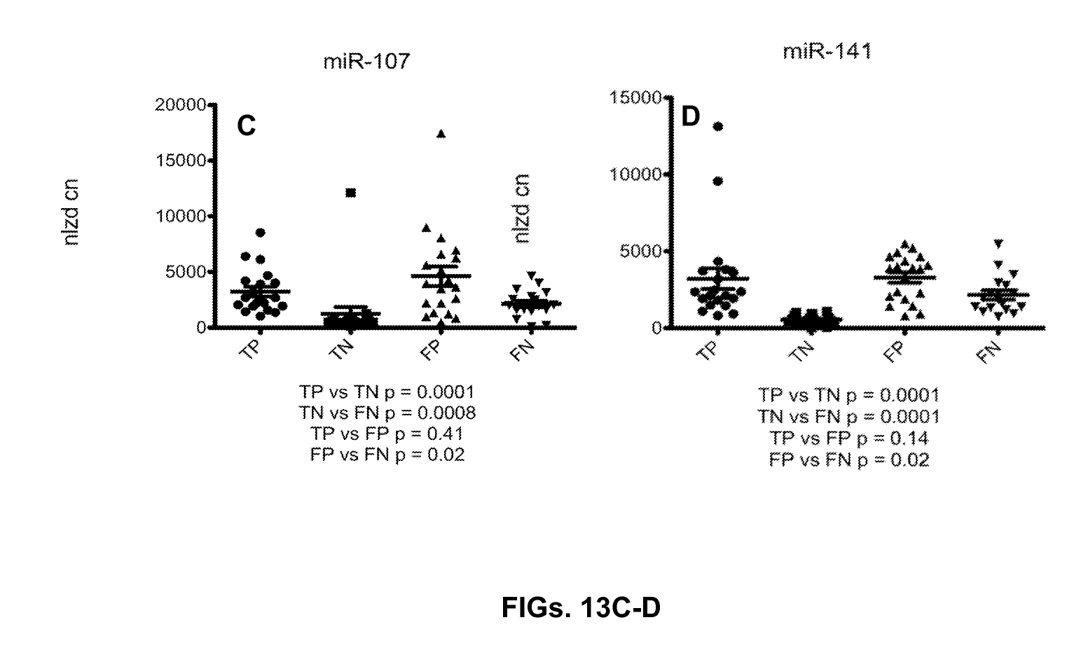

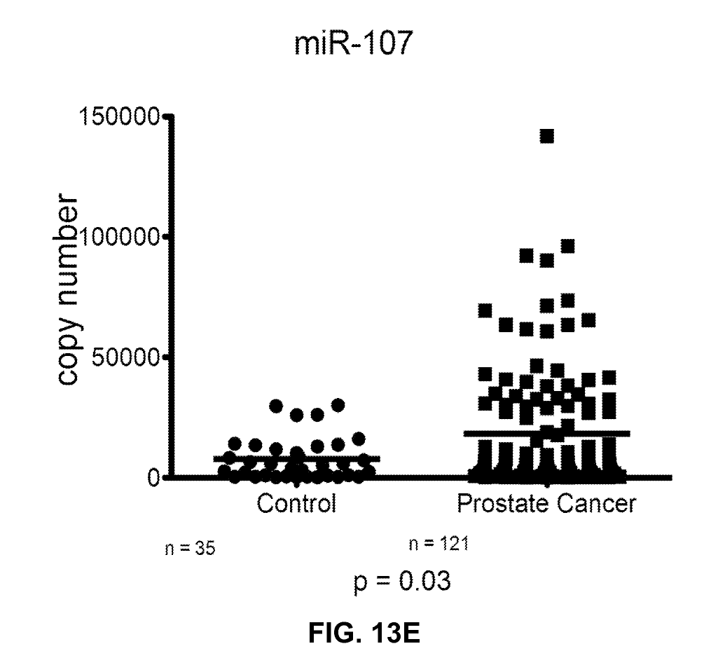

As described herein, vesicles are concentrated in plasma patient samples and assessed to provide a diagnostic, prognostic or theranostic readout. Vesicle analysis of patient samples includes the detection of vesicle surface biomarkers, e.g., surface antigens, and/or vesicle payload, e.g., mRNAs and microRNAs, as described herein. The payload within the vesicles can be assessed to enhance assay performance. For example, FIG. 13A illustrates a scheme for using miR analysis within vesicles to convert false negatives into true positives, thereby improving sensitivity. In this scheme, samples called negative by the vesicle surface antigen analysis are further confirmed as true negatives or true positives by assessing payload with the vesicles. Similarly, FIG. 13B illustrates a scheme for using miR analysis within vesicles to convert false positives into true negatives, thereby improving specificity. In this scheme, samples called positive by the vesicle surface antigen analysis are further confirmed as true negatives or true positives by assessing payload with the vesicles.

A diagnostic test for prostate cancer includes isolating vesicles from a blood sample from a patient to detect vesicles indicative of the presence or absence of prostate cancer. See, e.g., Examples 20-23. The blood can be serum or plasma. The vesicles are isolated by capture with “capture antibodies” that recognize specific vesicle surface antigens. The surface antigens for the prostate cancer diagnostic assay include the tetraspanins CD9, CD63 and CD81, which are generally present on vesicles in the blood and therefore act as general vesicle biomarkers, the prostate specific biomarkers PSMA and PCSA, and the cancer specific biomarker B7H3. The capture antibodies are tethered to fluorescently labeled beads, wherein the beads are differentially labeled for each capture antibody. Captured vesicles are further highlighted using fluorescently labeled “detection antibodies” to the tetraspanins CD9, CD63 and CD81. Fluorescence from the beads and the detection antibodies is used to determine an amount of vesicles in the plasma sample expressing the surface antigens for the prostate cancer diagnostic assay. The fluorescence levels in a sample are compared to a reference level that can distinguish samples having prostate cancer. In this Example, microRNA analysis is used to enhance the performance of the vesicle-based prostate cancer diagnostic assay.

FIG. 13C shows the results of detection of miR-107 in samples assessed by the vesicle-based prostate cancer diagnostic assay. FIG. 13D shows the results of detection of miR-141 in samples assessed by the vesicle-based prostate cancer diagnostic assay. In the figure, normalized levels of the indicated miRs are shown on the Y axis for true positives (TP) called by the vesicle diagnostic assay, true negatives (TN) called by the vesicle diagnostic assay, false positives (FP) called by the vesicle diagnostic assay, and false negatives (FN) called by the vesicle diagnostic assay. As shown in FIG. 13C, the use of miR-107 enhances the sensitivity of the vesicle assay by distinguishing false negatives from true negative (p=0.0008). FIG. 13E shows verification of increased miR-107 in plasma cMVs of prostate cancer patients compared to patients without prostate cancer using a different sample cohort. Similarly, FIG. 13D also shows that the use of miR-141 enhances the sensitivity of the vesicle assay by distinguishing false negatives from true negative (p=0.0001). Results of adding miR-141 are shown in Table 18. miR-574-3p performs similarly.

TABLE 18
Addition of miR-141 to vesicle-based test for PCa

| | Without miR-141 | With miR-141 |
|---|---|---|
| Sensitivity | 85% | 98% |
| Specificity | 86% | 86% |

In this Example, vesicles are detected via surface antigens that are indicative of prostate cancer, and the performance of the signature is further bolstered by examining miRs within the vesicles, i.e., sensitivity is increased without negatively affecting specificity. This general methodology can be extended for any setting in which vesicles are profiled for surface antigens or other informative characteristic, then one or more additional biomarker is used to enhance characterization. Here, the one or more additional biomarkers are miRs. They could also comprise mRNA, soluble protein, lipids, carbohydrates and any other vesicle-associated biological entities that are useful for characterizing the phenotype of interest.

### Example 28

### Vesicle Isolation and Detection Methods

A number of technologies known to those of skill in the art can be used for isolation and detection of vesicles to carry out the methods of the invention in addition to those described above. The following is an illustrative description of several such methods.

Glass Microbeads.

Available as VeraCode/BeadXpress from Illumina, Inc. San Diego, Calif., USA. The steps are as follows:
1. Prepare the beads by direct conjugation of antibodies to available carboxyl groups.
2. Block non specific binding sites on the surface of the beads.
3. Add the beads to the vesicle concentrate sample.
4. Wash the samples so that unbound vesicles are removed.
5. Apply fluorescently labeled antibodies as detection antibodies which will bind specifically to the vesicles.
6. Wash the plate, so that the unbound detection antibodies are removed.
7. Measure the fluorescence of the plate wells to determine the presence the vesicles.

Enzyme Linked Immunosorbent Assay (ELISA). Methods of performing ELISA are well known to those of skill in the art. The steps are generally as follows:
1. Prepare a surface to which a known quantity of capture antibody is bound.
2. Block non specific binding sites on the surface.
3. Apply the vesicle sample to the plate.
4. Wash the plate, so that unbound vesicles are removed.
5. Apply enzyme linked primary antibodies as detection antibodies which also bind specifically to the vesicles.
6. Wash the plate, so that the unbound antibody-enzyme conjugates are removed.
7. Apply a chemical which is converted by the enzyme into a color, fluorescent or electrochemical signal.
8. Measure the absorbency, fluorescence or electrochemical signal (e.g., current) of the plate wells to determine the presence and quantity of vesicles.

Electrochemiluminescence Detection Arrays.

Available from Meso Scale Discovery, Gaithersburg, Md., USA:
1. Prepare plate coating buffer by combining 5 mL buffer of choice (e.g. PBS, TBS, HEPES) and 75 μL of 1% Triton X-100 (0.015% final).
2. Dilute capture antibody to be coated.
3. Prepare 5 μL of diluted a capture antibody per well using plate coating buffer (with Triton).
4. Apply 5 μL of diluted capture antibody directly to the center of the working electrode surface being careful not to breach the dielectric. The droplet should spread over time to the edge of the dielectric barrier but not cross it.
5. Allow plates to sit uncovered and undisturbed overnight.

The vesicle containing sample and a solution containing the labeled detection antibody are added to the plate wells. The detection antibody is an anti-target antibody labeled with an electrochemiluminescent compound, MSD SULFO-TAG label. Vesicles present in the sample bind the capture antibody immobilized on the electrode and the labeled detection antibody binds the target on the vesicle, completing the sandwich. MSD read buffer is added to provide the necessary environment for electrochemiluminescence detection. The plate is inserted into a reader wherein a voltage is applied to the plate electrodes, which causes the label bound to the electrode surface to emit light. The reader detects the intensity of the emitted light to provide a quantitative measure of the amount of vesicles in the sample.

Nanoparticles.

Multiple sets of gold nanoparticles are prepared with a separate antibody bound to each. The concentrated microvesicles are incubated with a single bead type for 4 hours at 37° C. on a glass slide. If sufficient quantities of the target are present, there is a colorimetric shift from red to purple. The assay is performed separately for each target. Gold nanoparticles are available from Nanosphere, Inc. of Northbrook, Ill., USA.

Nanosight.

A diameter of one or more vesicles can be determined using optical particle detection. See U.S. Pat. No. 7,751,053, entitled “Optical Detection and Analysis of Particles” and issued Jul. 6, 2010; and U.S. Pat. No. 7,399,600, entitled “Optical Detection and Analysis of Particles” and issued Jul. 15, 2010. The particles can also be labeled and counted so that an amount of distinct vesicles or vesicle populations can be assessed in a sample.

### Example 29

### KRAS Sequencing in CRC Cell Lines and Patient Samples

KRAS RNA was isolated from vesicles derived from CRC cell lines and sequenced. RNA was converted to cDNA prior to sequencing. Sequencing was performed on the cell lines listed in Table 19:

TABLE 19
CRC cell lines and KRAS sequence

| | DNA or | KRAS Genotype | KRAS Genotype |
|---|---|---|---|
| Cell Line | Vesicle cDNA | Exon 2 | Exon 3 |
| Colo 205 | Vesicle cDNA | Wild type (WT) | WT |
| Colo 205 | DNA | WT | WT |
| HCT 116 | Vesicle cDNA | c.13G > GA | WT |
| HCT 116 | DNA | c.13G > GA | WT |
| HT29 | Vesicle cDNA | WT | WT |
| Lovo | Vesicle cDNA | c.13G > GA | WT |
| Lovo | DNA | c.13G > GA | WT |
| RKO | Vesicle cDNA | WT | WT |
| SW 620 | Vesicle cDNA | c.12G > T | WT |

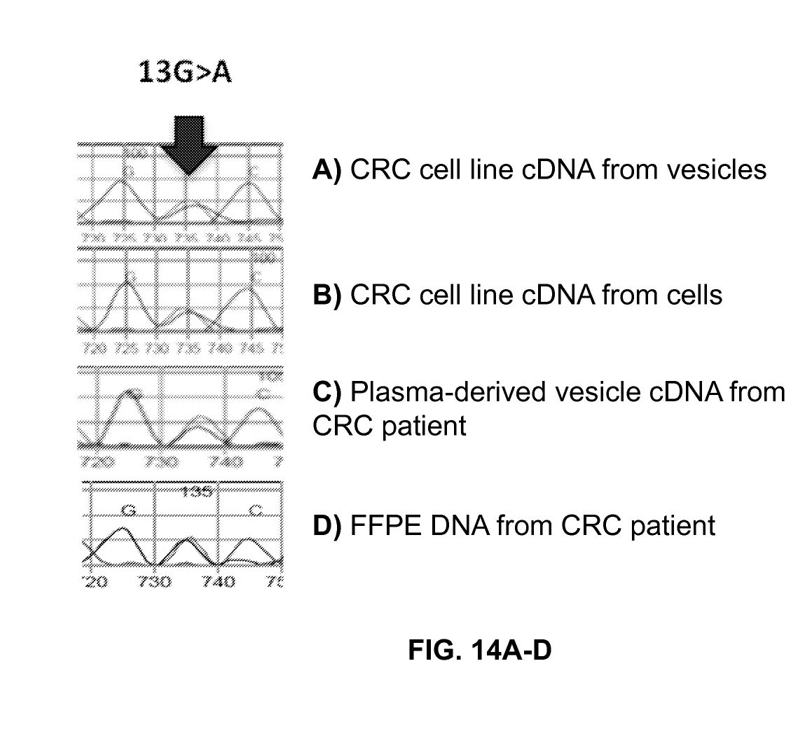

Table 19 and FIG. 14 show that the mutations detected in the genomic DNA from the cell lines was also detected in RNA contained within vesicles derived from the cell lines. FIG. 14 shows the sequence in HCT 116 cells of cDNA derived from vesicle mRNA in (FIG. 14A) and genomic DNA (FIG. 14B).

Twelve CRC patient samples were sequenced for KRAS. As shown in Table 20, all were wild type (WT). All patient samples received a DNase treatment during RNA Extraction. RNA was extracted from isolated vesicles. All 12 patients amplified for GAPDH demonstrating RNA was present in their vesicles.

TABLE 20
CRC patient samples and KRAS sequence

| | Sample | | KRAS Genotype | KRAS Genotype |
|---|---|---|---|---|
| Sample | Type | Stage | Exon 2 | Exon 3 |
| 61473a6 | Colon Ca | 1 | WT | WT |
| 62454a4 | Colon Ca | 1 | WT | WT |
| 110681a4 | Colon Ca | 1 | WT | Failed sequencing |
| 28836a7 | Colon Ca | 1 | WT | Failed sequencing |
| 62025a2 | Colon Ca | 2a | WT | WT |
| 62015a4 | Colon Ca | 2a | WT | WT |
| 110638a3 | Colon Ca | 2a | WT | WT |
| 110775a3 | Colon Ca | 2a | WT | WT |
| 35512a5 | Colon Ca | 3 | WT | WT |
| 73231a1 | Colon Ca | 2a | WT | WT |
| 85823a3 | Colon Ca | 3b | WT | WT |
| 23440a7 | Colon Ca | 3c | WT | WT |
| 145151A2/3 | Normal | | WT | WT |
| 139231A3 | Normal | | WT | Failed sequencing |
| 145155A4 | Normal | | WT | Failed sequencing |
| 145154A4 | Normal | | WT | Failed sequencing |

In a patient sample wherein the patient was found positive for the KRAS 13G>A mutation, the KRAS mutation from the tumor of CRC patient samples could also be identified in plasma-derived vesicles from the same patient. FIG. 14 shows the sequence in this patient of cDNA derived from vesicle mRNA in plasma (FIG. 14C) and also genomic DNA derived from a fresh frozen paraffin embedded (FFPE) tumor sample (FIG. 14D).

### Example 30

### Immunoprecipitation of Protein—Nucleic Acid Complexes

This Example examined the levels of miRNAs in plasma contained in complexes with Ago2, Apolipoprotein AI, and GW182. Specifically, miRNA levels were assessed after co-immunoprecipitation with antibodies to Ago2, Apolipoprotein AI, and GW182.

To carry out the immunoprecipitation, human plasma was incubated with antibodies bound to protein G beads against Ago2, Apolipoprotein AI, GW182, and an IgG control. To prepare the beads, 10 μg of anti-AGO2 (ab57113, lot GR29117-1, Abcam, Cambridge, Mass.), anti-ApoAI (PA1-22558, Thermo Scientific, Waltham, Mass.), anti-GW182 (A302-330A, Bethyl Labs, Montgomery, Tex.) or anti-IgG (sc-2025, Santa Cruz, Santa Cruz, Calif.) were conjugated to Magnabind protein G beads (Cat. #21349, Thermo Scientific) or Dynabead Protein G (Cat. #100.04D, Invitrogen, Carlsbad, Calif.). 200 μl of beads were placed in a 1.5 ml eppendorf tube and placed on a magnetic separator (Cat. # S1509S, New England Biolabs, Ipswich, Mass.) for one minute. The storage buffer was removed and discarded. The beads were washed once with 200 ml of phosphate buffered saline (PBS). The antibodies were allowed to bind the beads in 200 μl PBS for 30 minutes at room temperature (RT) and then for an additional 90 minutes at 4° C. The antibody-bound beads were placed on the magnetic separator for one minute. Unbound antibody was removed and discarded. The beads were washed three times with ice cold PBS.

The antibody conjugated beads were resuspended in 200 μl of PBS and mixed with 200 μl of human plasma from normal subjects (i.e., without cancer). The mixture was allowed to roll overnight on a Thermo Scientific Labquake Shaker/Rotisserie at 4° C. Following the overnight incubation, the beads were placed on the magnetic separator for 1 minute or until the solution turned clear. The beads were washed three times with 200 μl cold PBS and once with 200 μl of an NP-40 wash buffer (1% NP-40, 50 mM Tris-HCl, pH 7.4, 150 mM NaCl and 2 mM EDTA). Following the NP-40 buffer wash, the samples were rinsed one additional time with 200 μl of cold PBS. The beads were placed on the magnetic separator for one minute. The beads were the brought back to the original starting volume in 200 μl of PBS. Three quarters of the sample was used for RNA isolation as described previously (Arroyo et al., 2011). The remaining was stored at −20° C. for Western analysis.

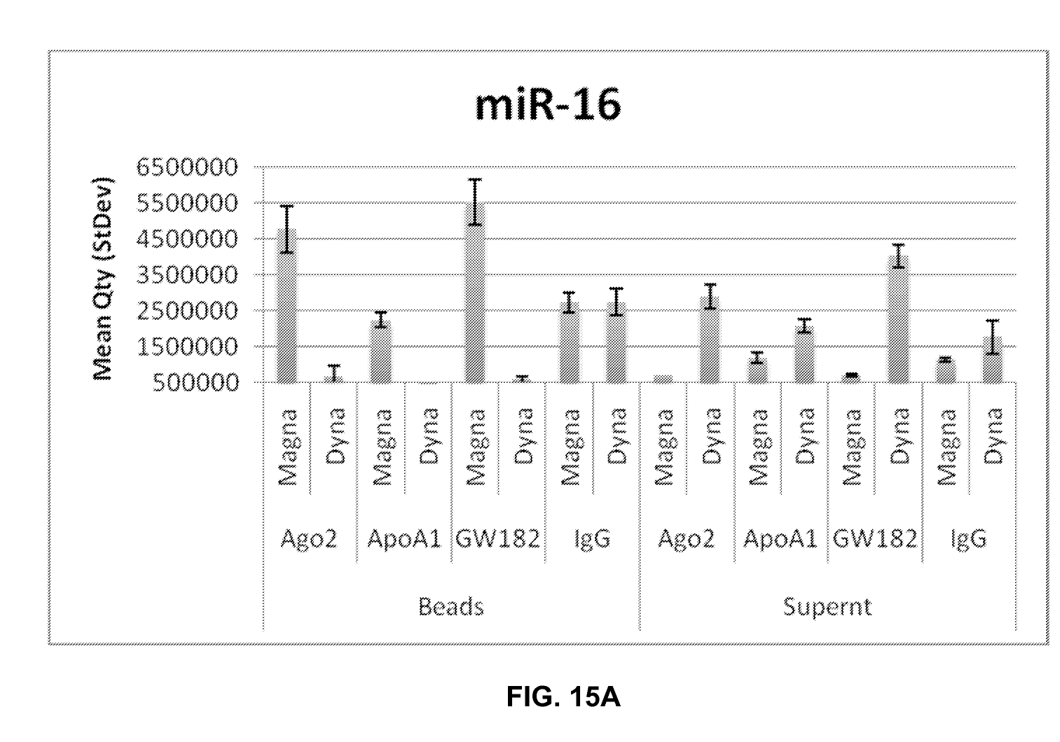

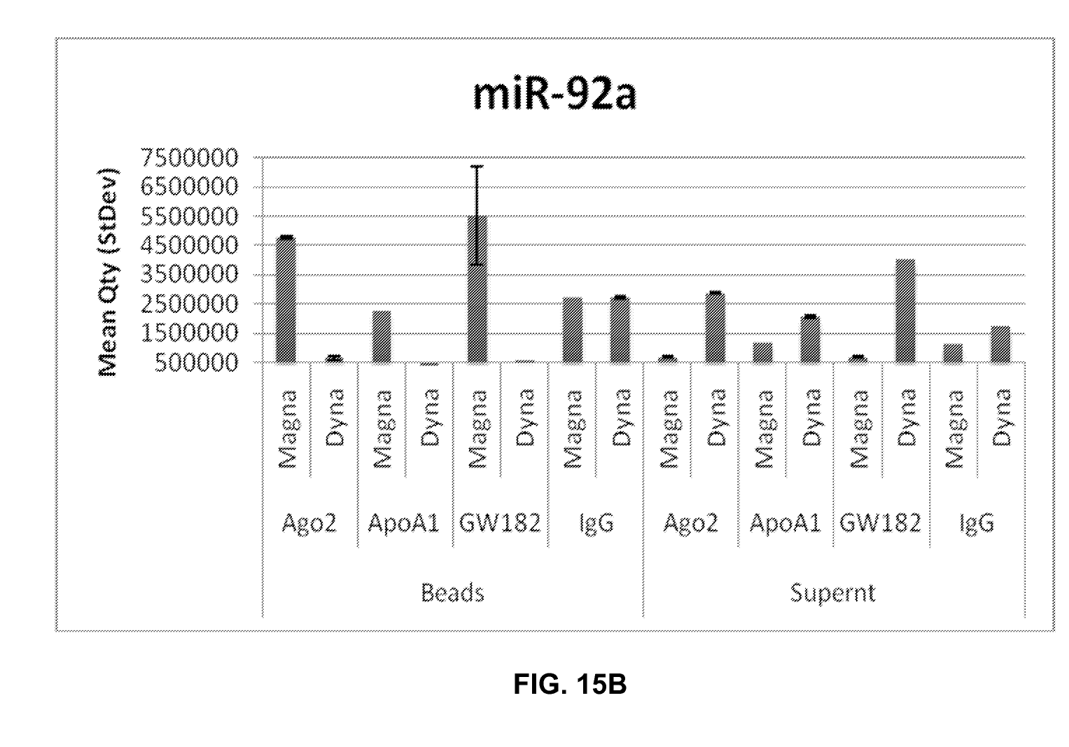

The isolated RNA was screened for miR-16 and miR-92a using ABI Taqman detection kits ABI_{—}391 and ABI_{—}431, respectively (Applied Biosystems, Carlsbad, Calif.). RNA was quantified against synthetic standards. The supernatant was collected and analyzed for selected miRNAs (miR-16 and miR-92a). The levels of miR-16 and miR-92a detected are shown in FIGS. 15A-B. As shown in the FIG. 15A and FIG. 15B, respectively, miR-16 and miR-92a co-immunoprecipitated with Ago2 and GW182 using Magnabeads at much higher levels than the IgG control (compare bars denoted as “Beads”). Co-immunoprecipitation with Dynabeads was unsuccessful for technical reasons which were not explored further.

Potential source(s) of miRNA from human plasma include vesicles and/or circulating Ago2-bound ribonucleoprotein complexes (RNP). miRs can be simultaneously isolated from complexes with AGO1-4 and vesicles using capture of GW182. This Example shows that miR-16 and miR-92a co-immunoprecipitate with AGO2 and GW182 in human plasma.

### Example 31

### Flow Analysis and Sorting of Cells, Vesicles and Protein-Nucleic Acid Complexes

This Example provides protocols for flow analysis and sorting of cells, circulating microvesicles (cMVs), and protein-nucleic acid complexes. Any appropriate antibody can be used that recognizes the markers of interest. The protocols can be applied to different sample sources, such as analysis of cells, vesicles and complexes from cell culture or from various bodily fluids.

1) Flow Sorting microRNA Complexes

Circulating microRNA derived from specific tissues can be isolated using tissue specific biomarkers to isolate the microvesicles and other microRNA complexes. This Example shows that microRNA in a PCSA/Ago2 double positive sub-population in human plasma can distinguish prostate cancer from non-cancer.

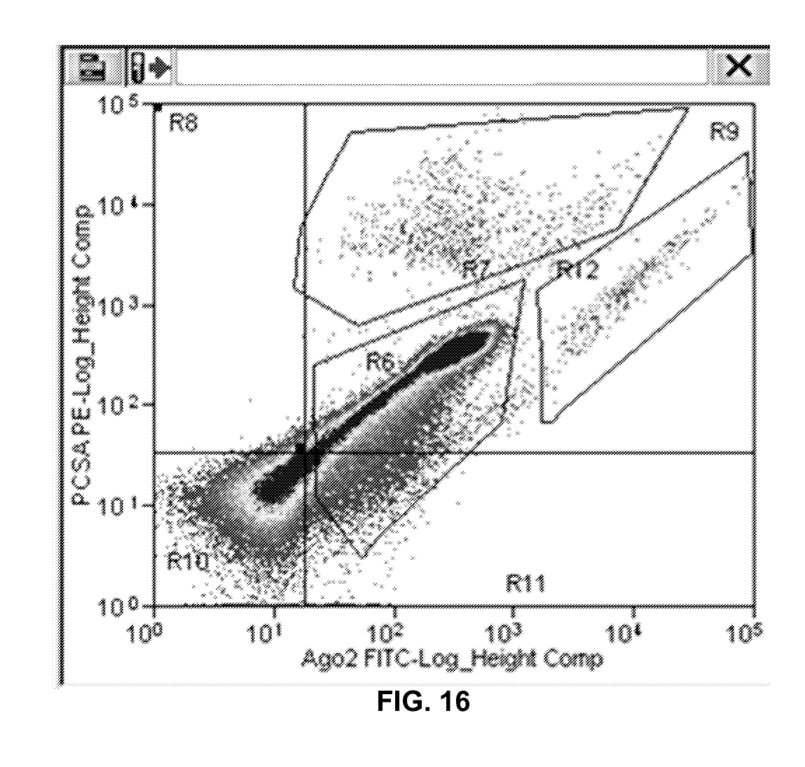

Plasma samples from three subjects with prostate cancer and three male subjects without prostate cancer were treated to concentrate vesicles as in Example 17. The concentrated vesicles were stained using optimized concentrations of antibodies against PCSA, a prostate specific biomarker, and Ago2 (ab57113, lot GR29117-1, Abcam, Cambridge, Mass.). The antibodies used were anti-PCSA labeled with PE and anti-Ago2 labeled with FITC. Positive gates were set using matching isotype control antibodies to define positive and negative regions. Sorted populations were selected based on regions as shown in FIG. 16. The Beckman Coulter MoFlo-XDP cell sorter and flow cytometer was used to isolated positive events using the high-purity sorting mode (i.e., “Purify 1/Drop”) to ensure that sorted events were pure to >90%. The MoFlo-XDP is capable of sorting two populations at rates of up to 50,000 events per second. To ensure purity and efficiency of the particle sort, the rate was between 200-300 events per second on average. Positive events were sorted into three 2 ml tubes and reserved for subsequent miR analysis.

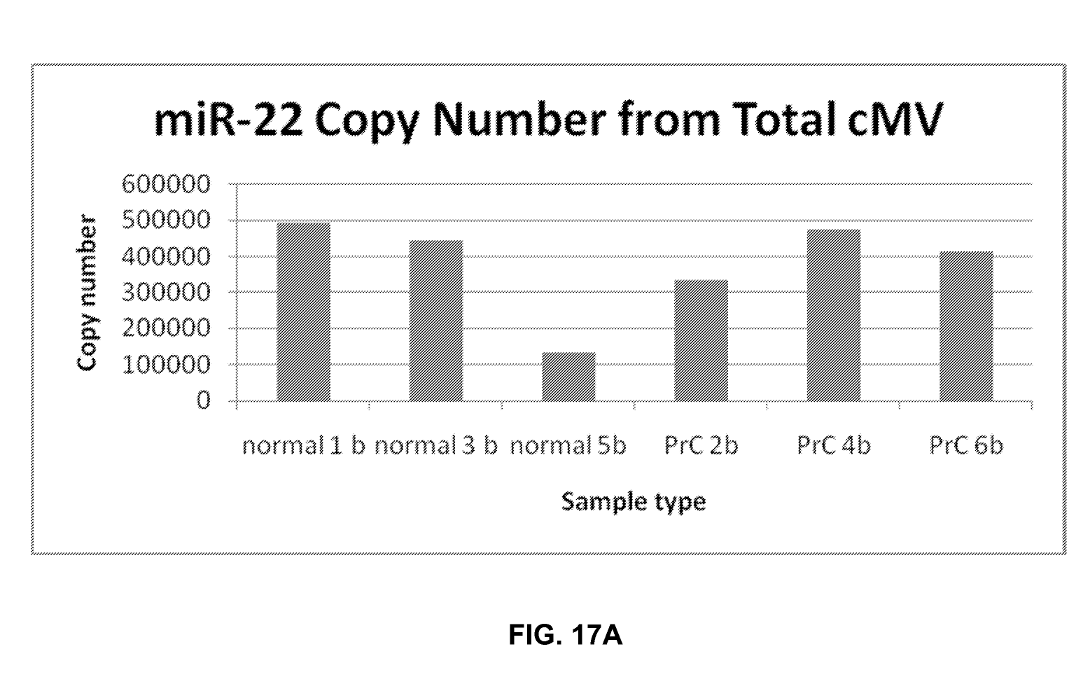

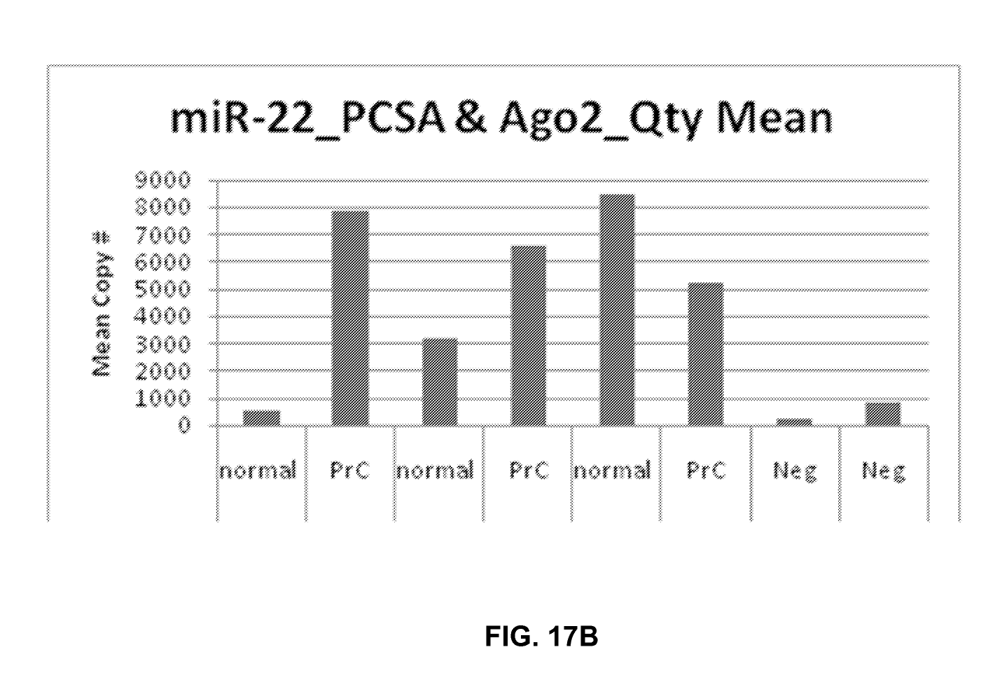

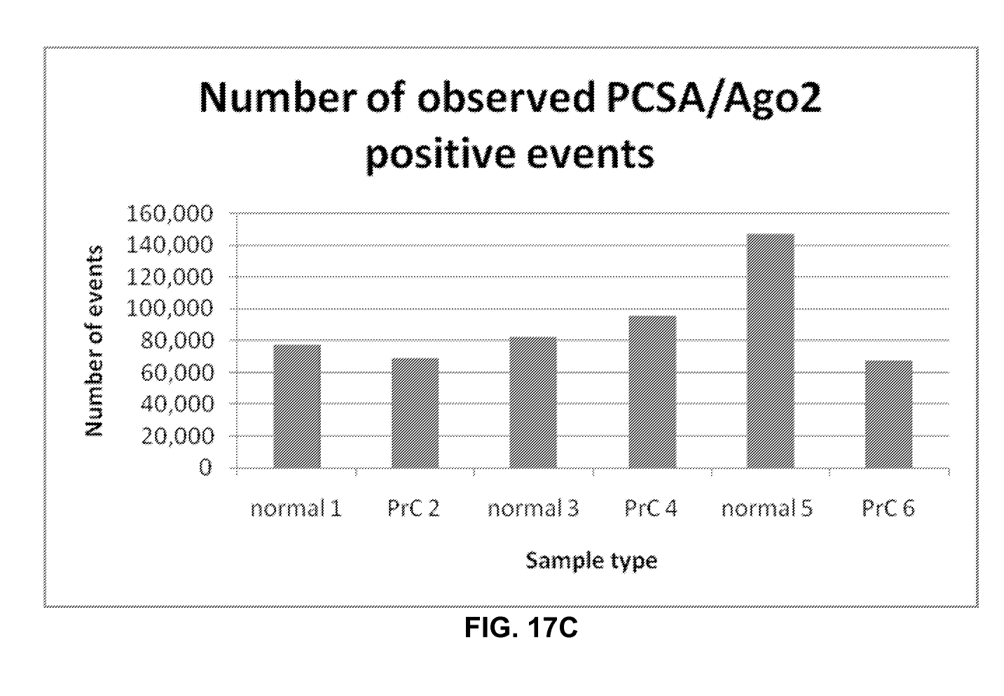

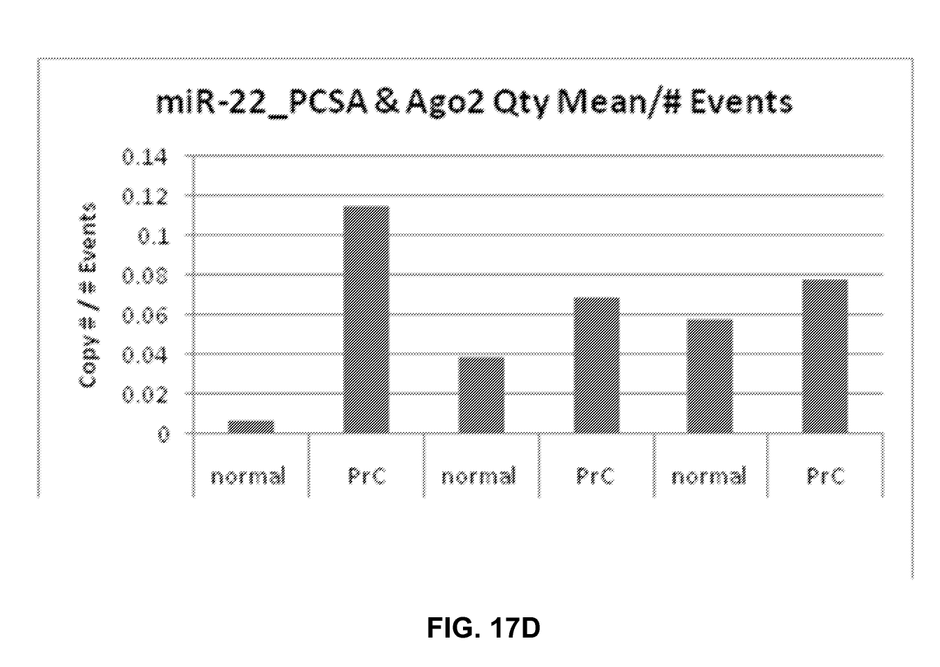

Once sorted, the microRNA content from each prostate specific subpopulation was evaluated. When a comparison of total concentrated plasma-derived microvesicles was made, little differential expression of miR-22 was observed between prostate cancer (PrC) and non-cancer samples (i.e., normals) (FIG. 17A). Similar results were observed with mean copy number levels of miR-22 from total RNA isolated from each PCSA/Ago2 double population (FIG. 17B). Without taking microRNA levels into account, the number of PCSA/Ago2 double positive events from each plasma sample did not significantly distinguish cancer from non-cancer (FIG. 17C). However, a clear separation was observed between prostate cancer and non-cancer when the number of observed copies of miR-22 from each sort was divided by the specific number of events from each sort (FIG. 17D). In this latter case, higher levels of miR-22 per PCSA/Ago2 double positive complexes were observed in all PCa plasma samples as compared to normal.

The protocol can be extended to detect and/or sort cMVs by detecting vesicles with anti-tetraspanin antibodies to first recognize cMVs. For example, the sample can first be sorted after staining with PE mouse anti-human CD9, BD 555372, PE mouse anti-human CD63, BD 556020, and PE mouse anti-human CD81, BD 555676. The sorted vesicles can then be assessed for PCSA/Ago as above.

2) Flow Sorting Cells and Vesicles

A Beckman Coulter MoFlo™ XDP flow cytometer and cell sorter was used to determine the expression of the indicated proteins on VCaP cells and VCaP vesicles. For cell staining, VCaP cells were detatched and washed in PBS. Approximately 3×10⁶ cells were resuspended in 1 ml Fc Block solution (Innovex Biosciences, part #NB309) and incubated at 4° C. for 10 minutes. 100 μl aliquots (3×10⁵ cells) were transferred to staining tubes, washed once in 5000 wash buffer (eBiosciences, cat #00-4222) and resuspended in 80-100 μl PBS-BN (phosphate buffered saline, pH6.4, 1% BSA and 0.05% Na-Azide) and pre-optimized concentration of the indicated antibodies. The antibody/cell solutions were incubated for 30 minutes at 4° C. in the dark, washed once in 100 μl of PBS-BN, resuspended in 250 μl of PBS-BN and analyzed in the MoFlo analyzer.

The cytometer was compensated before evaluation using commercially available compensation beads for FITC and PE with Summit Software integrated compensation software. For cells, the Gain for the linear FSC channel was 2.5 with linear SSC having voltage 491 and gain of 1.0, FL1 with voltage 433 and gain of 1.0 and FL2 with voltage 400 and gain of 1.0. For vesicles, the gain for FSC was increased to 3.5, the voltage for FL1 was increased to 501 and the voltage for FL2 was increased to 432 in order to increase detection of the smaller particles.

The Beckman Coulter MoFlo™ XDP flow cytometer and cell sorter was also used to sort various populations of vesicles in the following manner. Circulating MVs (cMVs) were stained using optimized concentrations of antibodies against the indicated proteins. Positive gates were set using matching isotype control antibodies to define positive and negative regions. The MoFlo sorter was used to isolated positive events using the high-purity sorting mode (i.e., “Purify 1 Drop”) to ensure that sorted events were pure to >90%. The MoFlo is capable of sorting two populations at rates of up to 50,000 events per second. For these sorts however, to ensure purity and efficiency of the particle sort, the rate was between 200-300 events per second on average. Subsequent evaluation using an aliquot of the sorted population rerun in the cytometer confirmed >90% purity of the population. Positive events are sorted into 2 ml tubes. The sorted vesicles can be used for further analysis, e.g., miR content within the sorted vesicles can be assessed.

### Example 32

### Protocol for Immunoprecipitation of Circulating Microvesicles

This Example provides a protocol for immunoprecipitation of circulating microvesicles (cMVs) from using antibodies to two markers. Any appropriate antibody can be used that will capture the desired vesicle markers of interest. The protocol can further be applied to different sample sources, such as analysis of vesicles from various bodily fluids. In this Example, prostate specific vesicles are double immunoprecipitated from plasma using antibodies to PCSA and CD9.
1) Thaw 1 ml plasma from a subject of interest. For example, a subject having prostate cancer or a control, such as a normal male without prostate cancer.
2) Stain the unconcentrated plasma with 40 μl anti-PCSA-PE conjugated antibody and 45 μl of anti-CD9-FITC to the plasma.
3) Mix and incubate for 30 minutes in the dark at room temperature.
4) Concentrate the plasma using 300kD columns from 1 ml to 300 μl to remove unbound antibodies.
5) Remove and set aside 50 μl of concentrated plasma to determine the starting content. Save for flow analysis, store 4° C.
6) Add 20 μl of anti-FITC microbeads to the remaining 250 μl of stained concentrate.
7) Incubate in the dark, refrigerated on a shaker for 30 mins.
8) Prepare MultiSort columns (Miltenyi Biotec Inc., Auburn, Calif.) by washing the columns with 3×100 μl washes with Separation Buffer (Miltenyi) off the magnet.
9) After the 30 minute incubation with anti-FITC microbeads (Miltenyi), dilute the stained and labeled plasma by adding 200 μl buffer to reduce viscosity. Dilute further if still too thick.
10) Add the ˜470 μl plasma solution to the top of a first washed column, column 1, sitting on the magnet.
11) Allow the plasma solution to flow through.
12) Add 2×100 μl washes to the upper reservoir to remove un-magnetized particles.
13) Total flow through for column 1 is ˜670 μl. Save for phenotyping.
14) Remove column 1 from the magnet.
15) Add 300 μl of buffer and plunge firmly to remove magnetized cMVs from column 1.
16) Add 10 μl Multisort Release Reagent (Miltenyi) to the retained volume (300 μl).
17) Mix and incubate 10 mins in the dark at 4° C.
18) An optional wash step can be performed to remove released microbeads as necessary.
19) Add 20 μl MultiSort Stop Reagent (Miltenyi) to the cMV solution.
20) Add 20 μl anti-PE MultiSort Beads (Miltenyi).
21) Mix and incubate 30 mins in the dark at 4° C.
22) Add the solution to the top of a second column, column 2, while on the magnet.
23) Allow to flow through and collect as flow through.
24) Add additional 100 μl to wash any un-magnetized particles off column 2 (˜450 μl).
25) Collect flow through and reserve for flow evaluation.
26) Remove column 2 from the magnet and add 300 μl buffer.
27) Plunge firmly to dislodge retained cells, reserve for flow evaluation.
28) Add 10 μl of Release Reagent to cleave the beads.
29) Incubate 10 mins in the dark at 4° C.
30) Add 20 μl Stop Reagent.
31) Move to flow evaluation.

Vesicles can also be immunoprecipitated in a sample using a single antibody and column step as desired. For example, prostate specific vesicles can be captured performing a single immunoprecipitation with anti-PSCA antibodies.

Flow Analysis.

Five populations collected above are analyzed by flow cytometry: 1) initial unseparated plasma; 2) flow through column 1; 3) retained column 1; 4) flow through column 2; and 5) retained column 2. All populations had CD9-FITC and anti-PCSA-PE added above. Beads were removed but the PE-conjugated antibodies remained on the cMVs and could be evaluated in the flow cytometer.
1) Transfer solutions of cMVs to TruCount tubes for quantification of cMVs/events.
2) Evaluate by flow cytometry using a Beckman Coulter MoFlo-XDP cell sorter. Calculate the number of events based on TruCount tubes (Beckman Coulter).

Other markers, such as listed in Table 5 herein, can be used for vesicle immunoprecipitation using this protocol. For example, vesicles have been immunoprecipitated using one or more of MFG-E8, PCSA, Mammaglobin, SIM2, NK-2R. The immunoprecipitated vesicles can be used for further analysis, e.g., determining vesicle levels or assessing other markers, e.g., surface antigens or payload, associated with the immunoprecipitated vesicles.

### Example 33

### Analysis of Protein, mRNA and microRNA Biomarkers in Circulating Microvesicles (cMVs)

Vesicles protein biomarkers are analyzed using a microsphere-based system. Selected antibodies to the target proteins of interest are conjugated to differentially addressable microspheres. See, e.g., methodology in Example 22. After conjugation, the antibody coated microspheres are washed, blocked by incubation in Starting Block Blocking Buffer in PBS (Catalog #37538, Thermo Scientific, a division of Thermo Fisher Scientific, Waltham, Mass.), washed in PBS and incubated with the concentrated cMVs from plasma as described below. Following capture of cMVs, the microsphere-cMV complexes are washed and incubated with phycoerythrin (PE) labeled detector antibodies to the tetraspanins CD9, CD63 and CD81 (i.e., PE labeled anti-CD9, PE labeled anti-CD63, and PE labeled anti-CD81) and washed prior to being detected on the microsphere reader. The fluorescent signal from 100 microspheres is measured and the median fluorescent intensity (MFI) for each differentially addressable microsphere—each corresponding to a different capture antibody—is calculated. Various combinations of detector and capture antibodies are examined in addition to the tetraspanin detectors described above.

Flow cytometry is used to determine the total number of cMVs in the patient samples. Patient plasma samples are diluted 100 times in PBS then incubated for 15 min at room temperature (RT) in BD Trucount tubes (BD Biosciences, San Jose, Calif.) for quantification of events per sample. Trucount tubes contain a known number of fluorescent beads that can be used to normalize events for each sample by flow cytometry. Sample acquisition by FACS Canto II cytometer (BD Biosciences) and analysis by FlowJo software (Tree Star, Inc., Ashland, Oreg.) are used to determine the number of sample events and number of Trucount beads per tube. Calculation of absolute number per sample is obtained following manufacturer's instructions (BD Biosciences) and adjustment by dilution factor as necessary.

MiRNAs are examined from the payload with cMVs from the plasma samples. cMVs are concentrated and the miRNAs are extracted using a modified Trizol method. Briefly, cMVs are treated with Rnase A (20 μg/ml for 20 min @ 37° C.; Epicentre®, an Illumina® company, Madison, Wis.) followed by Trizol treatment (750 μl of Trizol LS to each 100 μl) and vortexed for 30 min at 1400 rpm at room temperature. After centrifugation, the supernatant is collected and RNA is further purified with the miRNeasy 96 purification kit (Qiagen, Inc., Valencia, Calif.) and stored at −80° C. Forty ng of RNA are reverse transcibed and run on the Exiqon qRT-PCR Human panel I and II on an ABI 7900 (Applied Biosystems, life Technologies, Carlsbad, Calif.). See, e.g., Examples 13-14, 25. C_{T} values are calculated using SDS 2.4 software (Applied Biosystems). All samples are normalized to inter plate calibrator and RT-PCR control.

Messenger RNA (mRNA) is also examined in the cMV payload from the plasma samples. cMVs are isolated and treated with RNase A as above. mRNA is extracted using a modified Trizol method as above and purified with a Qiagen RNeasy mini kit precipitating with 70% ethanol (Qiagen, Inc.). The collected RNA is reverse transcribed and Cy-3 labeled using Agilent's “Low Input Quick Amp Labeling” kit for one-color gene expression analysis according to the manufacturer's instructions (Agilent Technologies, Santa Clara, Calif.). Labeled samples are hybridized to Agilent's Whole Genome 44K v2 arrays and washed according to manufacturer's specifications (Agilent Technologies). Arrays are scanned on an Agilent B scanner (Agilent Technologies) and data is extracted with Feature Extractor (Agilent Technologies) software. Extracted data is normalized with a global normalization method and analyzed with GeneSpring GX software (Agilent Technologies).

Both miRNA and messenger RNA can be examined from specific subpopulations of cMVs from the plasma. For example, cMVs are concentrated then the population that is positive for PCSA is isolated using immunoprecipitation. See Example 3. The PCSA+cMVs are isolated and miRNA and mRNA is isolated and analyzed as described above. The same methodology is used to examine the miRNA and mRNA content of vesicles isolated using different capture agents directed to different vesicle surface antigens of interest. In addition, the vesicles can be isolated that are positive for more than one surface antigen. See Example 32.

Normalized analyte values are imported into either R (available from The R Project for Statistical Computing at www.r-project.org) or SAS software (SAS Institute Inc., Cary, N.C.). The data is filtered using appropriate quality control measures and transformed prior to analysis. Analysis is performed as follows:

Signature Performance Evaluation (for Pre-Specified or Novel Signatures)

The sample sets generated using the methods above (i.e., payload analysis of isolated vesicle populations) can be used to evaluate the performance of a bio signature that is fully specified prior to either the unblinding of clinical outcome or to the unblinding of clinical laboratory testing of samples. In such a case, the signature is considered pre-specified and must be applied, unmodified, to new analyte data on this sample set to obtain predicted outcomes for all samples. Performance of the pre-specified signature is evaluated by comparing predicted and true outcome (for example, in terms of diagnostic sensitivity, specificity, and accuracy). Statistics include performance estimates and confidence intervals.

For signatures that are not pre-specified (i.e. that are derived with foreknowledge of both clinical outcome and laboratory testing results of samples), these samples may still be used to evaluate the performance of the signature. However, to reduce potentially biased estimates of performance, statistical analyses are performed nested within a k-fold cross validation loop that includes marker selection and class prediction steps as described below.

Marker Selection for Novel Signatures

Markers are included in novel signatures if they are statistically informative by testing for their association with disease outcome using a subset of commonly applied techniques known to those of skill in the art. These include: 1) Welch test—robust parametric statistical test for difference between group means when variances are unequal; 2) Wilcoxon signed-rank test—robust non-parametric statistical test that can be interpreted as showing an improvement in ROC AUC (above 0.50); 3) Youden's J—calculated as the maximum combined sensitivity and specificity for a marker, across all possible diagnostic thresholds. Statistical significance is evaluated via permutation tests.

Markers are judged statistically informative if the test is significant in the context of the number statistical tests performed. More specifically, comparison-wise p-values are adjusted for multiple testing—e.g. using false discovery rate thresholds or by control of family-wise error rates.

Formation of Novel Signatures

Once a subset of informative markers is identified in the marker selection stage described above, novel multi-marker models are formed using well-established modeling techniques. Parameters for signatures are estimated by training the models on the full training data set, and performance for the signature is evaluated as described under “Signature performance evaluation” using the approach “for signatures that are not prespecified.” Simple and well-established modeling techniques are used in these steps, including: discriminant analysis, support vector machines, logistic regression, and decision trees. Results for all models will be reported and optimal markers panels are identified accordingly.

Additional a posteriori analyses are performed on the data set for clinical variables of interest as available. Such variables include age, ethnicity, PSA levels, digital rectal exam (DRE) results, number of previous biopsies, indication for biopsy and biopsy result (e.g. HGPIN, ATYPIA, BPH, prostatitis or prostate cancer), and the like. Such analyses are performed by introducing covariates or stratification variables into previously defined models. P-values are corrected for multiple testing.

### Example 34

### Biological Pathway Expression in Circulating Microvesicles (cMVs)

In this Example, expression profiling of mRNA payload in cMVs is performed. Pathway analysis of mRNAs expressed in the cMVs is performed to identify the most significant biological pathways.

To profile mRNAs in whole vesicle populations, cMVs were isolated from 1 ml of plasma from three prostate cancer and three non-cancer control samples using filtration and concentration as described in Example 20. RNA was extracted from 100 μl of plasma concentrate, which was then subdivided into 25 μl aliquots for lysis with Trizol LS (Invitrogen, by life technologies, Carlsbad, Calif.) after treatment with RNASE A. The aqueous phase from each of the four aliquots was precipitated with 70% ethanol, combined on a single Qiagen mini RNA extraction column (Qiagen, Inc., Valencia, Calif.), and eluted in a 30 μl volume. The eluted RNA can be difficult to reliably quantify by standard means. Thus, a 10 μl volume was used for the subsequent labeling reactions. Samples were cy-3 labeled with “Low Input Quick Amp Labeling” kit from Agilent for one-color gene expression analysis according to the manufacturer's instructions (Agilent Technologies, Santa Clara, Calif.), with the following modifications: 1) The spike-in mix for Cy3 labeling was altered so that the third dilution was 1:5 and 1 μl was added to each sample; 2) 10 μl of sample was reduced in volume to 2.5 μl using a vacufuge in duplicate for each sample; 3) Every sample was processed in duplicate throughout the protocol until the purification step of the amplified samples. At the beginning of the purification protocol, the duplicate samples were combined and subsequently passed through the column; 4) The samples were not quantified after purification but rather the full volume of the purified sample was hybridized to the array. Labeled samples were then hybridized to Agilent Whole Genome 44K microarrays according to manufacturer's instructions (Agilent Technologies). Data was extracted with Feature Extractor software (Agilent Technologies) and analyzed with GeneSpring GX (Agilent Technologies). 4291 mRNAs were found to be present in the concentrate. The GeneSpring software was used to identify pathways that correlated with the expression patterns. Following the above analysis, the androgen receptor (AR) and EGFR1 pathways were the most significantly expressed pathways in the vesicle population. The members of the AR and EGFR1 pathways are shown in Table 21:

TABLE 21
Pathway Expression in Total cMVs

| Pathway | Members |
|---|---|
| Androgen | GTF2F1, CTNNB1, PTEN, APPL1, GAPDH, CDC37, |
| Receptor (AR) | PNRC1, AES, UXT, RAN, PA2G4, JUN, BAG1, |
| | UBE2I, HDAC1, COX5B, NCOR2, STUB1, HIPK3, |
| | PXN, NCOA4 |
| EGFR1 | RALBP1, SH3BGRL, RBBP7, REPS1, SNRPD2, |
| | CEBPB, APPL1, MAP3K3, EEF1A1, GRB2, RAC1, |
| | SNCA, MAP2K3, CEBPA, CDC42, SH3KBP1, CBL, |
| | PTPN6, YWHAB, FOXO1, JAK1, KRT8, RALGDS, |
| | SMAD2, VAV1, NDUFA13, PRKCB1, MYC, JUN, |
| | RFXANK, HDAC1, HIST3H3, PEBP1, PXN, TNIP1, |
| | PKN2 |

In a related set of experiments, expression profiling was performed in PCSA+cMVs. PCSA+cMVs were isolated using immunoprecipitation as in Example 32. Expression was performed as above using Agilent Whole Genome 44K microarrays. 2402 mRNAs were found in the PCSA captured samples. The TNF-alpha pathway was the most significantly overexpressed pathway. The members of the TNF-alpha pathway are shown in Table 22.

TABLE 22
Pathway Expression in PCSA+ cMVs

| Pathway | Members |
|---|---|
| TNF- | BCL3, SMARCE1, RPS11, CDC37, RPL6, RPL8, PAPOLA, |
| alpha | PSMC1, CASP3, AKT2, MAP3K7IP2, POLR2L, TRADD, |
| | SMARCA4, HIST3H3, GNB2L1, PSMD1, PEBP1, HSPB1, |
| | TNIP1, RPS13, ZFAND5, YWHAQ, COMMD1, COPS3, |
| | POLR1D, SMARCC2, MAP3K3, BIRC3, UBE2D2, |
| | HDAC2, CASP8, MCM7, PSMD7, YWHAG, NFKBIA, |
| | CAST, YWHAB, G3BP2, PSMD13, FBL, RELB, YWHAZ, |
| | SKP1, UBE2D3, PDCD2, HSP90AA1, HDAC1, KPNA2, |
| | RPL30, GTF2I, PFDN2 |

### Example 35

### Microarray Profiling of mRNA from Circulating Microvesicles (cMVs)

Large scale screening on high density arrays or mRNA levels within cMVs can be hindered by sample quantity and quality. A protocol was developed to allow robust analysis of cMV payload mRNAs that distinguish prostate cancer from normals.

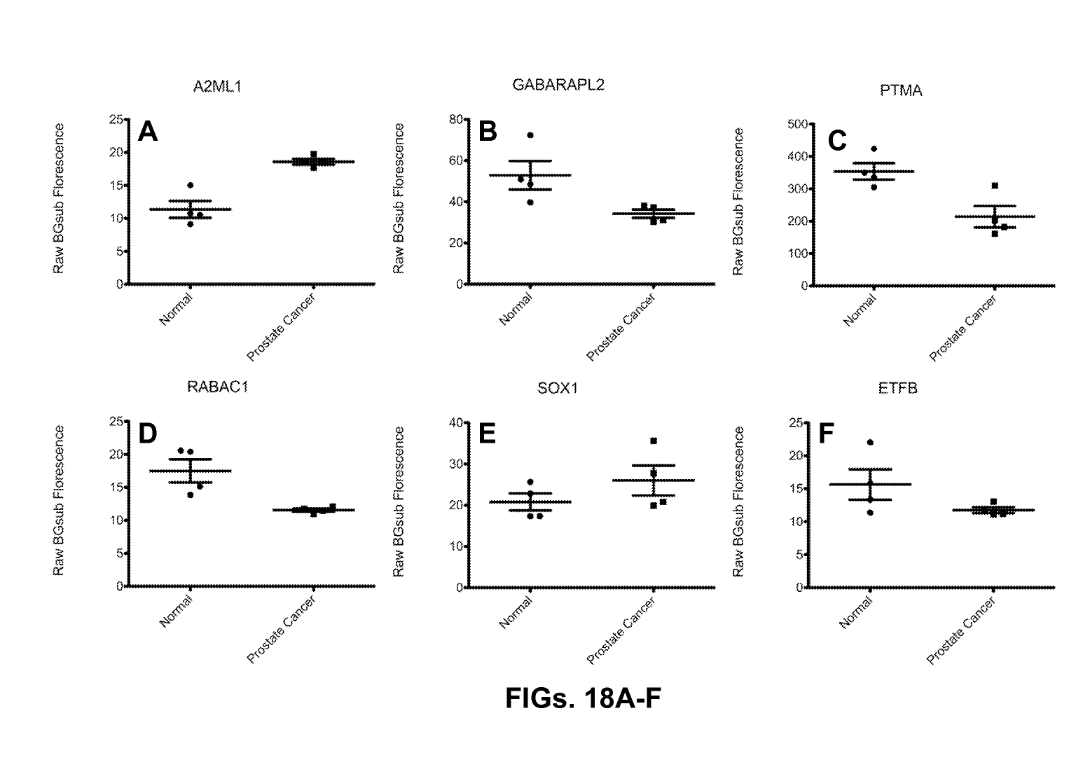

cMVs were isolated from 1 ml of plasma from four prostate cancer and four non-cancer control samples using filtration and concentration as described in Example 20. RNA was extracted from 100 μl of plasma concentrate, which was then subdivided into 25 μl aliquots for lysis with Trizol LS (Invitrogen, by life technologies, Carlsbad, Calif.) after treatment with RNASE A. The aqueous phase from each of the four aliquots was precipitated with 70% ethanol, combined on a single Qiagen mini RNA extraction column (Qiagen, Inc., Valencia, Calif.), and eluted in a 30 μl volume. The eluted RNA can be difficult to reliably quantify by standard means. Thus, a 10 μl volume was used for the subsequent labeling reactions. Samples were cy-3 labeled with “Low Input Quick Amp Labeling” kit from Agilent for one-color gene expression analysis according to the manufacturer's instructions (Agilent Technologies, Santa Clara, Calif.), with the following modifications: 1) The spike-in mix for Cy3 labeling was altered so that the third dilution was 1:5 and 1 μl was added to each sample; 2) 10 μl of sample was reduced in volume to 2.5 μl using a vacufuge in duplicate for each sample; 3) Every sample was processed in duplicate throughout the protocol until the purification step of the amplified samples. At the beginning of the purification protocol, the duplicate samples were combined and subsequently passed through the column; 4) The samples were not quantified after purification but rather the full volume of the purified sample was hybridized to the array. Labeled samples were then hybridized to Agilent Whole Genome 44K microarrays according to manufacturer's instructions (Agilent Technologies). Data was extracted with Feature Extractor software (Agilent Technologies) and analyzed with GeneSpring GX (Agilent Technologies). Genes with expression in at least 50% of the samples were included in the final analysis. 2155 probes were detected that met these criteria. Of these 2155, 24 were found to have significantly different expression (p value<0.05) between the prostate cancer group and the control group. See Table 23 and FIGS. 18A-F. Table 23 shows 24 genes that were significantly differently expressed between the mRNA payload from cMVs in the four prostate cancer patient samples and four healthy control samples. FIG. 18 shows dot plots of raw background subtracted fluorescence values of selected genes from the microarray: FIG. 18A shows A2ML1; FIG. 18B shows GABARAPL2; FIG. 18C shows PTMA; FIG. 18D shows RABAC1; FIG. 18E shows SOX1; FIG. 18F shows ETFB.

TABLE 23
Differentially expressed mRNAs in
cMVs from PCa and healthy samples

| GeneSymbol | p-value | Change in normal | FCAbsolute |
|---|---|---|---|
| A2ML1 | 0.001 | down | 1.88 |
| GABARAPL2 | 0.002 | up | 1.36 |
| PTMA | 0.002 | up | 1.76 |
| ETFB | 0.003 | up | 1.16 |
| RPL22 | 0.008 | down | 1.36 |
| GUK1 | 0.009 | up | 1.28 |
| PRDX5 | 0.011 | up | 1.48 |
| HIST1H3B | 0.014 | up | 1.29 |
| RABAC1 | 0.022 | up | 1.33 |
| PTMA | 0.024 | up | 1.65 |
| C1orf162 | 0.026 | down | 1.35 |
| HLA-A | 0.031 | up | 1.23 |
| SEPW1 | 0.033 | up | 1.31 |
| SOX1 | 0.034 | down | 1.38 |
| EIF3C | 0.034 | down | 1.30 |
| GZMH | 0.037 | up | 1.81 |
| CSDA | 0.040 | up | 1.79 |
| SAP18 | 0.040 | down | 1.36 |
| BAX | 0.043 | up | 1.20 |
| RABGAP1L | 0.045 | up | 2.19 |
| C10orf47 | 0.047 | down | 1.58 |
| HSP90AA1 | 0.047 | up | 1.46 |
| PTMA | 0.048 | up | 1.52 |
| NRGN | 0.049 | up | 2.57 |

Abbreviations in Table 23: “GeneSymbol” references nomenclature available for each gene feature on the array. Details for each gene are available from Agilent (www.chem.agilent.com) or the HUGO database (www.genenames.org). “FCAbsolute” shows absolute fold-change in mRNA levels detected between groups.

### Example 36

### Data Mining to Identify Biomarkers

MicroRNAs are known to regulate the expression of mRNA. An expression database has been created that contains information about the mRNA expression of many tumor types. The database contains data obtained using the Illumina DASL microarray (Illumina, Inc., San Diego, Calif.) for many thousands of patients. Circulating microvesicles (cMVs) contain microRNA as the dominant RNA species and also contain mRNAs. In this Example, an association was made between mRNA differentially expressed in cancer tumors from the expression database and those expressed in cMVs. The mRNAs found differentially expressed in tumor tissue were also used to find microRNA targets in cMVs.

Gene expression data from the expression database was evaluated to find the most statistically significant differentially expressed genes between prostate (PCa+), breast (BrCa), lung (LCa) and colorectal cancers (CRC) and matched normal tissue (PCa−), as well as between the cancer types (Table 24). Expression data (versions HT-12 and REF-8) for cancer samples (prostate, colorectal, breast, and lung) were analyzed to detect genes differentially expressed between cancer types. Similarly, prostate cancer (PCa) samples were compared against prostate normal samples to detect prostate cancer specific probes. To perform the analysis, expression data were normalized prior to analysis by adopting a subset of 20 arbitrarily selected arrays (6 breast cancer, 5 colorectal cancer, 5 lung cancer, and 4 prostate cancer) to generate a quantile reference distribution. All arrays in the data set were then normalized against the reference distribution to ensure that each array shared the same quantile distribution. Next, normalized expression data were analyzed for each probe in the data set. Differentially expressed probes (and their corresponding genes) were detected by comparing each pair of classes (e.g. prostate cancer vs. breast cancer, and prostate cancer vs. prostate normal) using a F-score (a.k.a. Fisher's score) statistic. This statistic, which measures between vs. within class variation, was obtained by calculating the square of the mean group difference over the square of the sum of the group standard deviations. F-scores were set negative where the mean for the PCa+ samples was the lower of the two groups. Lastly, F-scores were sorted into descending sequence using the absolute value of the F-score, and the top up/down regulated markers were chosen from the list.

TABLE 24
Most Statistically Significant Differentially Expressed
Genes Between PCa+ Samples and Indicated Samples

| Rank | PCa− | BrCa | CRC | LCa | |
|---|---|---|---|---|---|
| 1 | SEMG1 | KLK2 | KLK2 | KLK2 | |
| 2 | MAP4K1 | KLK2 | KLK2 | KLK2 | |
| 3 | CXCL13 | MAOA | KLK4 | LRRC26 | |
| 4 | GNAO1 | KLK4 | LRRC26 | LOC389816 | PCA+ |
| | | | | | Lower |
| 5 | DST | PVRL3 | CDX1 | KLK4 | PCA+ |
| | | | | | Higher |
| 6 | AQP2 | SLC45A3 | EEF1A2 | CAB39L | |
| 7 | NELL2 | NLGN4Y | FOXA2 | SPDEF | |
| 8 | TNNT3 | STX19 | SPDEF | SIM2 | |
| 9 | PRSS21 | CYorf14 | BAIAP2L2 | SLC45A3 | |
| 10 | SNAI2 | C22orf32 | FAM110B | PNPLA7 | |
| 11 | BMP5 | PNPLA7 | MIPOL1 | TRIM36 | |
| 12 | PGF | SIM2 | CEACAM6 | GSTP1 | |
| 13 | POU3F1 | FEV | SLC45A3 | TRPV6 | |
| 14 | ERCC1 | TRPM8 | ADRB2 | ASTN2 | |
| 15 | TAF1C | ARG2 | LOC389816 | MUC1 | |
| 16 | KLHL5 | TRIM36 | C19orf33 | MUC1 | |
| 17 | C16orf86 | ADRB2 | ZNF613 | ZNF613 | |
| 18 | SMARCD3 | LRRC26 | TRIM36 | FAM110B | |
| 19 | PENK | EIF1AY | ERN2 | FEV | |
| 20 | SCML1 | SLC30A4 | TRIM31 | CRIP1 | |

For prostate cancer, a list of the most significantly over and under-expressed genes was generated. These genes were compared to a list of mRNA that had been detected in cMVs from prostate cancer patients via microarray. One gene from the tissue list, AQP2, was also found to be expressed in cMVs. The list of up- and down-regulated genes from prostate tumor tissue was then mined using the TargetScan public database for microRNA that may influence the expression of these mRNAs. Matching microRNA was found for 11 of the 20 mRNA examined (Table 25). This list of microRNAs was then compared to a list of microRNAs that we found to be reliably detected in cMVs. This comparison revealed that 10 of the microRNAs that regulate the mRNA of interest in the prostate tumor tissue are also found in cMVs (Table 25).

TABLE 25
microRNA associated with differentially expressed mRNAs

| | TargetScan | Detected | | TargetScan | Detected |
|---|---|---|---|---|---|
| PCa Up | result | in cMV? | PCa Down | result | in cMV? |
| ADCYAP1R1 | no target | n/a | SEMG1 | no target | n/a |
| HECTD3 | miRs-26a + b | yes | MAP4K1 | miR-342-5p | no |
| SLC44A4 | no target | n/a | CXCL13 | miR-186 | yes |
| FASN | miRs-15/16/ | yes | GNAO1 | miR-1271 | no |
| | 195/497/424 | | | | |
| MPG | no target | n/a | DST | miR-600 | no |
| MIR720 | no target | n/a | AQP2 | miR-216b | no |
| PTBP1 | miR-206 | yes | NELL2 | miR-519 | no |
| | | | | family | |
| CPSF1 | no target | n/a | TNNT3 | no target | n/a |
| C2orf56 | no target | n/a | PRSS21 | miR-206 | yes |
| HCRTR1 | no target | n/a | SNAI2 | miR-203 | yes |

Additionally, mRNAs that are found to be differentially expressed are often indicative of differences in the protein level. The results of this mining activity have identified proteins (e.g., KLK2) associated with cMVs that can be used to differentiate prostate cancer from other cancers, including breast, lung, and colon cancer. KLK2 is known to be associated with prostatic tissue.

### Example 37

### Circulating Microvesicles (cMVs) in Prostate Cancer Patient Samples

In this Example, cMVs are profiled in prostate cancer and related diseases. Generally, capture antibodies were tethered to fluorescently labeled microbeads and incubated with cMVs from patient plasma samples. The captured cMVs were detected with fluorescently labeled detector antibodies. Fluorescent signals are then used to compare levels of specific cMV populations in the patient samples. A total of 216 patient samples were included in the study, including 91 cancers and 125 non-cancers. All subjects had either a biopsy result of cancer and any subject with a negative result from a ≧10 core biopsy. Patient blood samples were clarified at 3000×g in a Labofuge centrifuge before cMVs were isolated from 1 mL of plasma by filtration (see Example 20 for more details). Thirty samples that failed to pass quality measures were removed from further data analysis. Characteristics of 175 samples that passed quality controls are shown in Table 26. Eleven additional samples were collected from normals with no known prostate disorders but were not used in the comparisons in this Example.

TABLE 26
Patient Characteristics

| Pathology Type | Number |
|---|---|
| Benign Prostate Disorder | 48 |
| Benign with Inflammation | 27 |
| High Grade Pin (HGPIN) | 15 |
| Prostatic atypia/Atypical small acinar | 8 |
| proliferation (ASAP) | |
| Cancer First Biopsy | 71 |
| Cancer Watchful Waiting | 6 |

Capture and detector binding agents are shown in Table 27:

TABLE 27
Capture and Detector Antibodies

| Binding Agent | Target | Vendor | Catalog# | Lot# |
|---|---|---|---|---|
| Anti filamin A alpha antibody | FLNA | Sigma-Aldrich | WH0002316M1 | 11165-51 |
| Anti TNF-related apoptosis-inducing ligand | Trail-R4 | R&D systems | MAB633 | DQQ0209121 |
| receptor 4 antibody | | | | |
| Anti human Versican antibody | VCAN | R&D systems | MAB3054 | UGW0209061 |
| Anti-cluster of differentiation 9 antibody | CD9 | R&D Systems | MAB1880 | JOK0610081 |
| Anti synovial sarcoma, X breakpoint 4 | SSX4 | Novus | H00006759- | 11237-3E10 |
| antibody | | Biologicals | MO2 | |
| Anti CD3 antibody [OKT3] | CD3 | Abcam | ab86883 | GR52307-1 |
| Anti carbohydrate 19-9 antibody | CA-19-9 | US Biological | C0075-13B | L10122109 |
| Anti membrane spanning 4A1 antibody | MS4A1 | Sigma | WH0000931M1 | 091114-5C11 |
| Anti carcino embryogenic antibody | CD66e CEA | US Biological | C1300-08 | L11081075 |
| Anti Mucin17, cell surface associated | MUC17 | Santa Cruz | sc32602 | I0309 |
| protein antibody | | | | |
| Anti epidermal growth factor antibody | EGFR | BD biosciences | 555996 | 17563 |
| Anti receptor activator of NFκB antibody | RANK | R&D systems | MAB683 | EDV0209071 |
| Anti-Chondroitin sulfate antibody | CSA | abcam | ab11570 | GR18185-5 |
| Anti Prostate specific membrane antibody | PSMA | Biolegend | 342502 | B132497 |
| Anti human inactive complement | iC3b | Thermo | MA1-82814 | MG1439545 |
| component 3b antibody | | | | |
| Anti chicken IgY antibody (NON-HPLC) | Antichicken | Abcam | ab50579 | GR41703-6 |
| | IgY | | | |
| Anti Cluster of differentiation 276 antibody | B7H3 | R&D systems | MAB1027 | HPA0410081 |
| Anti prostate cell surface antibody | PCSA | Inhouse | Inhouse | H10G006b |
| Anti cluster of differentiation 63 antibody | CD63 | BD pharmingen | 556019 | 82575 |
| Anti Mucin 1, cell surface associated protein | MUC1 | Santa Cruz | sc7313 | E2510 |
| antibody | | | | |
| Anti Transglutaminase-2 antibody | TGM2 | Sigma Aldrich | WH0007052M10 | 08309-2F4 |
| Anti cluster of differentiation 81 antibody | CD81 | BD pharmingen | 555675 | 54545 |
| Anti S100 calcium binding protein A4 | S100-A4 | Sigma aldrich | WH0006275M1 | 11222- |
| antibody | | | | S1/11210-S1 |
| Anti Milk fat globule-EGF factor 8 protein | MFG-E8 | R&D systems | MAB27671 | WQK0111031 |
| antibody | | | | |
| Anti-Human granulocyte macrophage | GM-CSF | Invitrogen | AHC2012 | 642599A |
| colony stimulating factor antibody | | | | |
| Anti Integrin a5 (A-11) antibody | Integrin | Santacruz | sc-166665 | H0410 |
| Anti Neurokinin-A antibody | NK-2R(C-21) | Santacruz | sc-14121 | J0103 |
| Anti Prostate specific antibody | PSA | Novus | NB100-66506 | 300611 |
| | | Biologicals | | |
| Anti Cluster of differntiation 24 antibody | CD24 | BD biosciences | bd 555426 | 5483 |
| (Heat Stable antigen) | | | | |
| Anti Human Epidermal growth factor | HER3 (ErbB3) | US Biological | E3451-36A | L11092051 |
| Receptor 3 antibody | | | | |
| Anti Tissue inhibitor of metallo proteinase-1 | TIMP-1 | Sigma-Aldrich | WH0007076M1 | 11025-4D12 |
| antibody | | | | |
| Anti human interleukin 6 unconjugated | IL6 Unc | Invitrogen | AHC0762 | 706056A |
| antibody | | | | |
| Anti Prostatic binding protein antibody | PBP | Novus | H00005037-M01 | 10264- |
| | | Biologicals | | S3/10236-2G2 |
| Anti Apoptotic linked gene product 2 | ALIX | Thermo | MA1-83977 | MG1439546 |
| Interacting Protein X antibody | | scientific | | |
| | | pierce | | |
| Anti Matrixmetallo Proteinase 9 antibody | MMP9 | Novus | NBP1-28617 | K3205-V421 |
| | | biologicals | | |
| Anti prolactin Monoclonal antibody | PRL | Thermo | MA1-10597 | MG1439591 |
| | | Scientific | | |
| | | Pierce | | |
| Anti Ephrin-A receptor 2 antibody | EphA2 | Santa Cruz | sc101377 | K0409 |
| Anti cytidine and dCMP deaminase domain | CDADC1 | Sigma-Aldrich | WH0081602M1 | 11251-1A2 |
| containing 1 antibody | | | | |
| Anti Matrix metallo Proteinase 7 antibody | MMP7 | Novus | NB300-1000 | J10902 |
| | | biologicals | | |
| Anti c-reactive protein antibody | CRP | Abcam | ab13426 | GR15824-6 |
| Anti saccharomyces cerevisiae antibody | ASCA | abcam | ab19731 | 880975 |
| Anti runt-related transcription factor 2 | RUNX2 | Sigma aldrich | WH0000860M1 | 10138-1D8 |
| antibody | | | | |
| Anti Tumor necrosis factor like weak | TWEAK | US biological | T9185-01 | L11081013 |
| inducer of apoptosis | | | | |
| Anti serpin peptidase inhibitor, clade B | SERPINB3 | Sigma aldrich | WH0006317M1 | 10155-2F5 |
| member 3 antibody | | | | |
| Anti cytokeratin 19 fragment antibody | CYFRA21-1 | MedixMab | 102221 | 24594 |
| Anti mammaglobin A(C-16) antibody | Mammaglobin | Santa Cruz | sc-48328 | B2107 |
| Anti Vascular endothelial growth factor A | VEGF A | US Biological | V2110-05D | L10112413 |
| antibody | | | | |
| Anti surfactant protein-C antibody | SPC | US Biological | U2575-03 | L10100604 |
| Anti Interleukin-1B antibody | IL-1B | Sigma Aldrich | WH0003553M1 | 10264-2A8 |
| Anti tumor protein 53 antibody | p53 | BioLegend | 645802 | B136322 |
| Anti glyco protein a33 antibody | A33 | Santa Cruz | sc33014 | I0911 |
| Anti Aurora Bkinase (serine/threonine- | AURKB | Novus | H00009212- | 11223-6A6 |
| protein kinase 6) antibody | | Biologicals | M01A | |
| Anti cluster of differentiation 41 antibody | CD41 | Mybiosource | MBS210248 | n/a |
| Anti Chemokine (C-X-C motif) ligand 12 | CXCL12 | R&D systems | MAB350 | COJ0510101 |
| antibody | | | | |
| Anti X antigen family, member 1 antibody | XAGE | Santa cruz | sc-134820 | B2210 |
| Anti SAM pointed domain containing ets | SPDEF | Novus | H00025803-M01 | 7285-4A5- |
| transcription factor antibody | | Biologicals | | 00LcY6/11081-4A5 |
| Anti Interleukin 8 antibody | IL8 | Thermo | OMA1-03346 | MG1439681 |
| | | scientific | | |
| | | pierce | | |
| Anti B-cell novel protein1 antibody | BCNP | abcam | ab59781 | GR49524-1 |
| Anti Alpha-methylacyl-CoA racemase | AMACR | Novus | H00023600-M02 | 11228-1D8 |
| antibody | | biological | | |
| Anti human decorin antibody | DCRN | R&D systems | MAB143 | EC10209061 |
| Anti GATA binding protein 2 antibody | GATA2 | Sigma-Aldrich | WH0002624M1 | 10271-2D11 |
| Anti seprase antibody | seprase/FAP | R&D | MAB3715 | CCHZ0109071 |
| Anti Neutrophil gelatinase-associated | NGAL | Santa Cruz | sc50350 | F0710 |
| lipocalin antibody | | | | |
| Anti Epithelial cellular adhesion molecule | EpCAM | R&D systems | MAB 9601 | UTT0911061 |
| antibody | | | | |
| Anti Galactose metabolism regulator 3 | GAL3 | Santa Cruz | sc-32790 | D0910 |
| antibody | | | | |
| Anti proviral integration site antibody | PIM1 | Novus | H00005292-M08 | 11020-1C10 |
| | | Biologicals | | |
| Anti tumor susceptibility gene 101 antibody | Tsg 101 | Santacruz | sc-101254 | I1310 |
| Anti single minded protein 2 antibody | SIM2 (C-15) | Santacruz | sc-8715 | G2810 |
| Anti Flagellin antibody | C-Bir (Flagellin) | abcam | ab93713 | GR35089-5 |
| Anti Six Transmembrane Epithelial Antigen | STEAP | Santacruz | sc-25514 | H2707/A0204 |
| of the Prostate 1 antibody | | | | |
| Anti heat shock protein antibody | HSP70 | Biolegend | 648002 | B130984 |
| Anti Vascular Endothelial Growth Factor | hVEGFR2 | R&D systems | MAB3572 | HHV0810011 |
| Receptor 2 antibody | | | | |
| Anti Ets related gene antibody | ERG | sigma aldrich | SAB2500363 | 7081P1 |
| Anti autoimmunogenic cancer/testis antigen | NY-ESO-1 | US biological | N8590-01 | L11080550 |
| Anti Mucin 2, cell surface associated protein | MUC2 | Santa Cruz | sc15334 | B1811/G2111 |
| antibody | | | | |
| Anti disintegrin and metalloproteinase | ADAM10 | R&D systems | MAB1427 | HZR0310021 |
| domain 10 antibody | | | | |
| Anti Aspartyl/asparaginyl β- | ASPH (A-10) | Santa Cruz | sc-271391 | B1411 |
| hydroxylase(A10) antibody | | | | |
| Anti carbohydrate antigen 125 antibody | CA125 | US Biological | C0050-01D | L11060368 |
| (MUC16) | | | | |
| Anti TNF-related apoptosis-inducing ligand | TRAIL R2 | Thermo | PA1-23497 | MC1399147 |
| receptor 2 antibody | | scientific | | |
| | | pierce | | |
| Anti Human gro alpha antibody | Gro alpha | GeneTex | GTX10376 | 26629 |
| Anti kallikrein-related peptidase 2 antibody | KLK2 | Novus | H00003817-M03 | 08130-3C5 |
| | | Biologicals | | |
| Anti synovial sarcoma X break point 2 | SSX2 | Novus | H00006757-M01 | 11223-1A4 |
| antibody | | biologicals | | |

PE-labeled antibodies to five detector agents were used, comprising: 1) EpCam; 2) CD81 alone; 3) PCSA; 4) MUC2; and 5) MFG-E8. Combinations of detector agents along with microbead-tethered capture agents are shown in Table 28. In the table, the capture and/or detector agents comprised antibodies that recognize to the indicated targets unless noted as aptamers. The first row identifies the Detector agents. Beneath each detector is the list of capture agents used with the detector. Chicken IgY was run as a control.

TABLE 28
Capture and Detector Agent Combinations

| EpCam | CD81 | PCSA | MUC2 | MFG-E8 |
|---|---|---|---|---|
| FLNA | FLNA | FLNA | FLNA | FLNA |
| Trail-R4 | Trail-R4 | Trail-R4 | Trail-R4 | Trail-R4 |
| VCAN | VCAN | VCAN | VCAN | VCAN |
| CD9 | CD9 | CD9 | CD9 | CD9 |
| SSX4 | SSX4 | SSX4 | SSX4 | SSX4 |
| CD3 | CD3 | CD3 | CD3 | CD3 |
| CA-19-9 | CA-19-9 | CA-19-9 | CA-19-9 | CA-19-9 |
| MS4A1 | MS4A1 | MS4A1 | MS4A1 | MS4A1 |
| CD66e CEA | CD66e CEA | CD66e CEA | CD66e CEA | CD66e CEA |
| MUC17 | MUC17 | MUC17 | MUC17 | MUC17 |
| EGFR | EGFR | EGFR | EGFR | EGFR |
| RANK | RANK | RANK | RANK | RANK |
| CSA | CSA | CSA | CSA | CSA |
| PSMA | PSMA | PSMA | PSMA | PSMA |
| iC3b | iC3b | iC3b | iC3b | iC3b |
| Chicken IgY | Chicken IgY | Chicken IgY | Chicken IgY | Chicken IgY |
| B7H3 | B7H3 | B7H3 | B7H3 | B7H3 |
| PCSA | PCSA | PCSA | PCSA | PCSA |
| CD63 | CD63 | CD63 | CD63 | CD63 |
| MUC1 | MUC1 | MUC1 | MUC1 | MUC1 |
| TGM2 | TGM2 | TGM2 | TGM2 | TGM2 |
| CD81 | CD81 | CD81 | CD81 | CD81 |
| S100-A4 | S100-A4 | S100-A4 | S100-A4 | S100-A4 |
| MFG-E8 | MFG-E8 | MFG-E8 | MFG-E8 | MFG-E8 |
| GM-CSF | GM-CSF | GM-CSF | GM-CSF | GM-CSF |
| Integrin | Integrin | Integrin | Integrin | Integrin |
| NK-2R(C-21) | NK-2R(C-21) | NK-2R(C-21) | NK-2R(C-21) | NK-2R(C-21) |
| PSA | PSA | PSA | PSA | PSA |
| CD24 | CD24 | CD24 | CD24 | CD24 |
| HER3 (ErbB3) | HER3 (ErbB3) | HER3 (ErbB3) | HER3 (ErbB3) | HER3 (ErbB3) |
| TIMP-1 | TIMP-1 | TIMP-1 | TIMP-1 | TIMP-1 |
| IL6 Unc | IL6 Unc | IL6 Unc | IL6 Unc | IL6 Unc |
| PBP | PBP | PBP | PBP | PBP |
| ALIX | ALIX | ALIX | ALIX | ALIX |
| MMP9 | MMP9 | MMP9 | MMP9 | MMP9 |
| PRL | PRL | PRL | PRL | PRL |
| EphA2 | EphA2 | EphA2 | EphA2 | EphA2 |
| CDADC1 | CDADC1 | CDADC1 | CDADC1 | CDADC1 |
| MMP7 | MMP7 | MMP7 | MMP7 | MMP7 |
| CRP | CRP | CRP | CRP | CRP |
| ASCA | ASCA | ASCA | ASCA | ASCA |
| RUNX2 | RUNX2 | RUNX2 | RUNX2 | RUNX2 |
| TWEAK | TWEAK | TWEAK | TWEAK | TWEAK |
| SERPINB3 | SERPINB3 | SERPINB3 | SERPINB3 | SERPINB3 |
| CYFRA21-1 | CYFRA21-1 | CYFRA21-1 | CYFRA21-1 | CYFRA21-1 |
| Mammaglobin | Mammaglobin | Mammaglobin | Mammaglobin | Mammaglobin |
| VEGF A | VEGF A | VEGF A | VEGF A | VEGF A |
| SPC | SPC | SPC | SPC | SPC |
| IL-1B | IL-1B | IL-1B | IL-1B | IL-1B |
| p53 | p53 | p53 | p53 | p53 |
| A33 | A33 | A33 | A33 | A33 |
| AURKB | AURKB | AURKB | AURKB | AURKB |
| CD41 | CD41 | CD41 | CD41 | CD41 |
| CXCL12 | CXCL12 | CXCL12 | CXCL12 | CXCL12 |
| XAGE | XAGE | XAGE | XAGE | XAGE |
| SPDEF | SPDEF | SPDEF | SPDEF | SPDEF |
| IL8 | IL8 | IL8 | IL8 | IL8 |
| BCNP | BCNP | BCNP | BCNP | BCNP |
| AMACR | AMACR | AMACR | AMACR | AMACR |
| DCRN | DCRN | DCRN | DCRN | DCRN |
| GATA2 | GATA2 | GATA2 | GATA2 | GATA2 |
| seprase/FAP | seprase/FAP | seprase/FAP | seprase/FAP | seprase/FAP |
| NGAL | NGAL | NGAL | NGAL | NGAL |
| EpCAM | EpCAM | EpCAM | EpCAM | EpCAM |
| GAL3 | GAL3 | GAL3 | GAL3 | GAL3 |
| PIM1 | PIM1 | PIM1 | PIM1 | PIM1 |
| Tsg 101 | Tsg 101 | Tsg 101 | Tsg 101 | Tsg 101 |
| SIM2 (C-15) | SIM2 (C-15) | SIM2 (C-15) | SIM2 (C-15) | SIM2 (C-15) |
| C-Bir (Flagellin) | C-Bir (Flagellin) | C-Bir (Flagellin) | C-Bir (Flagellin) | C-Bir (Flagellin) |
| STEAP | STEAP | STEAP | STEAP | STEAP |
| HSP70 | HSP70 | HSP70 | HSP70 | HSP70 |
| hVEGFR2 | hVEGFR2 | hVEGFR2 | hVEGFR2 | hVEGFR2 |
| ERG | ERG | ERG | ERG | ERG |
| NY-ESO-1 | NY-ESO-1 | NY-ESO-1 | NY-ESO-1 | NY-ESO-1 |
| MUC2 | MUC2 | MUC2 | MUC2 | MUC2 |
| ADAM10 | ADAM10 | ADAM10 | ADAM10 | ADAM10 |
| ASPH (A-10) | ASPH (A-10) | ASPH (A-10) | ASPH(A-10) | ASPH (A-10) |
| CA125 | CA125 | CA125 | CA125 | CA125 |
| TRAIL R2 | TRAIL R2 | TRAIL R2 | TRAIL R2 | TRAIL R2 |
| Gro alpha | Gro alpha | Gro alpha | Gro alpha | Gro alpha |
| KLK2 | KLK2 | KLK2 | KLK2 | KLK2 |
| SSX2 | SSX2 | SSX2 | SSX2 | SSX2 |

25 μl of concentrated plasma was incubated with the antibody-conjugated microspheres for each detector/capture combination. In a parallel set of experiments, the anti-PCSA detector was also run with 3 μl of concentrated plasma was used for each capture. All samples were run in duplicate.

A number of different two-group comparisons were done to identify the capture/detector pair of markers (hereinafter “marker pairs”) best able to discriminate the groups as outlined in the following tables. The levels of the detected vesicles were compared between these groups using a non-parametric Kruskal-Wallace test corrected with Benjamini and Hochberg False Discovery Rate (“FDR”) or Bonferroni's correction (“Bonf”). Kruskal-Wallace is similar to analysis of variance with the data replaced by rank and is equivalent to the Mann-Whitney U test/Wilcoxon rank sum test when comparing two groups. Marker pairs with positive control data (PCa positive and negative pooled patient samples) that was indistinguishable from blank negative controls was excluded from further analysis. As another quality control measure, samples were excluded from analysis wherein the fluorescence values of vesicles captured using anti-CD9 antibody fall in the lower 5% of the data obtained using the CD81 detector. As the tetraspanins CD9 and CD81 are generally expressed on vesicles, this measure excludes sample with insufficient levels of vesicles. In the tables, detector “PCSA (25)” indicates samples where 25 μl of concentrated plasma was used with labeled anti-PCSA as a detector. Likewise, detector “PCSA (3)” indicates samples where 3 μl of concentrated plasma was used with labeled anti-PCSA as a detector.

Table 29 shows the top performing detector/capture combinations for distinguishing prostate cancer (PCa+) samples from all other samples (PCA−). In this comparison, PCa+ is defined as any previous (i.e., watchful waiting) or current (i.e., first) positive biopsy and PCA− is defined as all other biopsy outcomes. Raw and corrected p-values are shown in Table 29:

TABLE 29
All Positive Biopsies v All Negative Biopsies

| | | Effect | Wilcoxon | | |
|---|---|---|---|---|---|
| Detector | Capture | size | p-value | FDR | Bonf |
| Epcam | MMP7 | 0.8621 | 0.0000 | 0.0000 | 0.0000 |
| PCSA (25) | MMP7 | 0.7953 | 0.0000 | 0.0000 | 0.0000 |
| Epcam | BCNP | 0.7840 | 0.0000 | 0.0000 | 0.0000 |
| PCSA (25) | ADAM10 | 0.7589 | 0.0000 | 0.0000 | 0.0000 |
| PCSA (25) | KLK2 | 0.7544 | 0.0000 | 0.0000 | 0.0000 |
| PCSA (25) | SPDEF | 0.7471 | 0.0000 | 0.0000 | 0.0000 |
| PCSA (25) | IL-1B | 0.7427 | 0.0000 | 0.0000 | 0.0000 |
| PCSA (25) | EGFR | 0.7361 | 0.0000 | 0.0000 | 0.0001 |
| CD81 | MMP7 | 0.7303 | 0.0000 | 0.0000 | 0.0002 |
| PCSA (25) | CD9 | 0.7242 | 0.0000 | 0.0000 | 0.0003 |
| PCSA (25) | EpCAM | 0.7234 | 0.0000 | 0.0000 | 0.0004 |
| PCSA (25) | PBP | 0.7215 | 0.0000 | 0.0000 | 0.0004 |
| PCSA (25) | p53 | 0.7199 | 0.0000 | 0.0000 | 0.0005 |
| MFGE8 | MMP7 | 0.7181 | 0.0000 | 0.0001 | 0.0013 |
| PCSA (25) | SERPINB3 | 0.7091 | 0.0000 | 0.0001 | 0.0017 |
| PCSA (25) | SSX4 | 0.6985 | 0.0000 | 0.0003 | 0.0052 |
| PCSA (25) | SSX2 | 0.6967 | 0.0000 | 0.0003 | 0.0062 |
| PCSA (25) | HER3 (ErbB3) | 0.6967 | 0.0000 | 0.0003 | 0.0062 |
| PCSA (25) | AURKB | 0.6964 | 0.0000 | 0.0003 | 0.0064 |
| PCSA (25) | BCNP | 0.6934 | 0.0000 | 0.0004 | 0.0087 |
| PCSA (25) | CD24 | 0.6920 | 0.0000 | 0.0005 | 0.0099 |
| PCSA (25) | HSP70 | 0.6890 | 0.0000 | 0.0006 | 0.0133 |
| PCSA (3) | BCNP | 0.6888 | 0.0000 | 0.0006 | 0.0136 |
| PCSA (25) | TGM2 | 0.6881 | 0.0000 | 0.0006 | 0.0146 |
| PCSA (25) | CYFRA21-1 | 0.6862 | 0.0000 | 0.0007 | 0.0176 |

In Table 30, a subset of PCa+ and PCa− samples was compared. The samples met the following criteria: 1) Positive biopsy or negative biopsy with >10 cores; 2) 40≦age≦75; 3) 0≦serum PSA (ng/ml)≦10; and 4) no previous biopsies (either positive or negative). The sample cohort meeting this criteria is referred to as the “restricted sample set.”

TABLE 30
Restricted Positive Biopsies v Negative Biopsies

| | | Effect | Wilcoxon | | |
|---|---|---|---|---|---|
| Detector | Capture | size | p-value | FDR | Bonf |
| Epcam | MMP7 | 0.8947 | 0.0000 | 0.0000 | 0.0000 |
| Epcam | BCNP | 0.8310 | 0.0000 | 0.0000 | 0.0000 |
| PCSA (25) | MMP7 | 0.8125 | 0.0000 | 0.0000 | 0.0000 |
| PCSA (25) | ADAM10 | 0.7647 | 0.0000 | 0.0000 | 0.0002 |
| CD81 | MMP7 | 0.7568 | 0.0000 | 0.0001 | 0.0004 |
| PCSA (25) | SPDEF | 0.7510 | 0.0000 | 0.0001 | 0.0007 |
| PCSA (25) | IL-1B | 0.7505 | 0.0000 | 0.0001 | 0.0007 |
| PCSA (25) | EGFR | 0.7384 | 0.0000 | 0.0003 | 0.0022 |
| PCSA (25) | KLK2 | 0.7358 | 0.0000 | 0.0003 | 0.0027 |
| PCSA (25) | p53 | 0.7244 | 0.0000 | 0.0007 | 0.0074 |
| PCSA (25) | EpCAM | 0.7236 | 0.0000 | 0.0007 | 0.0080 |
| PCSA (25) | CD9 | 0.7227 | 0.0000 | 0.0007 | 0.0087 |
| PCSA (3) | BCNP | 0.7209 | 0.0000 | 0.0007 | 0.0101 |
| MFGE8 | MMP7 | 0.7286 | 0.0000 | 0.0007 | 0.0104 |
| PCSA (25) | AURKB | 0.7174 | 0.0000 | 0.0009 | 0.0136 |
| PCSA (25) | BCNP | 0.7110 | 0.0001 | 0.0014 | 0.0230 |
| PCSA (25) | PBP | 0.7070 | 0.0001 | 0.0019 | 0.0319 |
| PCSA (25) | CSA | 0.7024 | 0.0001 | 0.0025 | 0.0459 |
| CD81 | BCNP | 0.7007 | 0.0001 | 0.0028 | 0.0527 |
| Muc2 | PRL | 0.6986 | 0.0001 | 0.0030 | 0.0617 |
| PCSA (25) | SERPINB3 | 0.6984 | 0.0001 | 0.0030 | 0.0629 |
| PCSA (25) | ASCA | 0.6979 | 0.0001 | 0.0030 | 0.0654 |
| Muc2 | TIMP-1 | 0.6950 | 0.0002 | 0.0036 | 0.0818 |
| PCSA (25) | SSX2 | 0.6926 | 0.0002 | 0.0041 | 0.0981 |
| PCSA (25) | CA-19-9 | 0.6913 | 0.0002 | 0.0043 | 0.1079 |

In Table 31, a second subset of PCa+ and PCa− samples was compared. The samples met the following criteria: 1) Positive biopsy or negative biopsy with ≧10 cores; 2) 40≦age≦75; 3) 0≦serum PSA (ng/ml)≦10; and 4) no previous positive biopsies (but may have had previous negative biopsy). Note the criteria 4) differs from the cohort directly above.

TABLE 31
Restricted Positive Biopsies v Negative Biopsies

| | | Effect | Wilcoxon | | |
|---|---|---|---|---|---|
| Detector | Capture | size | p-value | FDR | Bonf |
| Epcam | MMP7 | 0.8975 | 0.0000 | 0.0000 | 0.0000 |
| Epcam | BCNP | 0.8278 | 0.0000 | 0.0000 | 0.0000 |
| PCSA (25) | MMP7 | 0.8252 | 0.0000 | 0.0000 | 0.0000 |
| PCSA (25) | ADAM10 | 0.7772 | 0.0000 | 0.0000 | 0.0000 |
| PCSA (25) | SPDEF | 0.7656 | 0.0000 | 0.0000 | 0.0001 |
| PCSA (25) | IL-1B | 0.7614 | 0.0000 | 0.0000 | 0.0001 |
| CD81 | MMP7 | 0.7568 | 0.0000 | 0.0000 | 0.0002 |
| PCSA (25) | EGFR | 0.7538 | 0.0000 | 0.0000 | 0.0002 |
| PCSA (25) | KLK2 | 0.7514 | 0.0000 | 0.0000 | 0.0003 |
| PCSA (25) | EpCAM | 0.7403 | 0.0000 | 0.0001 | 0.0008 |
| PCSA (25) | p53 | 0.7398 | 0.0000 | 0.0001 | 0.0009 |
| PCSA (25) | CD9 | 0.7373 | 0.0000 | 0.0001 | 0.0011 |
| PCSA (3) | BCNP | 0.7319 | 0.0000 | 0.0001 | 0.0018 |
| MFGE8 | MMP7 | 0.7385 | 0.0000 | 0.0002 | 0.0022 |
| PCSA (25) | BCNP | 0.7290 | 0.0000 | 0.0002 | 0.0024 |
| PCSA (25) | AURKB | 0.7279 | 0.0000 | 0.0002 | 0.0027 |
| PCSA (25) | PBP | 0.7255 | 0.0000 | 0.0002 | 0.0033 |
| PCSA (25) | ASCA | 0.7168 | 0.0000 | 0.0004 | 0.0074 |
| Muc2 | PRL | 0.7161 | 0.0000 | 0.0004 | 0.0079 |
| PCSA (25) | CSA | 0.7154 | 0.0000 | 0.0004 | 0.0084 |
| PCSA (25) | SERPINB3 | 0.7141 | 0.0000 | 0.0004 | 0.0094 |
| PCSA (25) | SSX2 | 0.7124 | 0.0000 | 0.0005 | 0.0110 |
| PCSA (25) | CYFRA21-1 | 0.7102 | 0.0000 | 0.0006 | 0.0133 |
| PCSA (25) | HER3 (ErbB3) | 0.7093 | 0.0000 | 0.0006 | 0.0143 |
| PCSA (25) | CA-19-9 | 0.7073 | 0.0000 | 0.0007 | 0.0170 |

Table 32 shows the results when comparing newly identified PCa+ versus all PCA− samples. This comparison excludes the watchful waiting samples.

TABLE 32
Newly Identified Positive Biopsies v Negative Biopsies

| | | Effect | Wilcoxon | | |
|---|---|---|---|---|---|
| Detector | Capture | size | p-value | FDR | Bonf |
| Epcam | MMP7 | 0.8767 | 0.0000 | 0.0000 | 0.0000 |
| PCSA (25) | MMP7 | 0.8108 | 0.0000 | 0.0000 | 0.0000 |
| Epcam | BCNP | 0.8018 | 0.0000 | 0.0000 | 0.0000 |
| PCSA (25) | ADAM10 | 0.7764 | 0.0000 | 0.0000 | 0.0000 |
| PCSA (25) | KLK2 | 0.7672 | 0.0000 | 0.0000 | 0.0000 |
| PCSA (25) | SPDEF | 0.7644 | 0.0000 | 0.0000 | 0.0000 |
| PCSA (25) | IL-1B | 0.7576 | 0.0000 | 0.0000 | 0.0000 |
| PCSA (25) | EGFR | 0.7525 | 0.0000 | 0.0000 | 0.0000 |
| PCSA (25) | CD9 | 0.7410 | 0.0000 | 0.0000 | 0.0001 |
| PCSA (25) | EpCAM | 0.7367 | 0.0000 | 0.0000 | 0.0001 |
| PCSA (25) | p53 | 0.7366 | 0.0000 | 0.0000 | 0.0001 |
| PCSA (25) | PBP | 0.7360 | 0.0000 | 0.0000 | 0.0002 |
| CD81 | MMP7 | 0.7350 | 0.0000 | 0.0000 | 0.0002 |
| PCSA (25) | SERPINB3 | 0.7208 | 0.0000 | 0.0001 | 0.0008 |
| MFGE8 | MMP7 | 0.7231 | 0.0000 | 0.0001 | 0.0013 |
| PCSA (25) | SSX2 | 0.7151 | 0.0000 | 0.0001 | 0.0015 |
| PCSA (25) | HER3 (ErbB3) | 0.7139 | 0.0000 | 0.0001 | 0.0017 |
| PCSA (25) | SSX4 | 0.7098 | 0.0000 | 0.0001 | 0.0026 |
| PCSA (25) | AURKB | 0.7091 | 0.0000 | 0.0001 | 0.0028 |
| PCSA (25) | BCNP | 0.7071 | 0.0000 | 0.0002 | 0.0034 |
| PCSA (25) | TGM2 | 0.7052 | 0.0000 | 0.0002 | 0.0041 |
| PCSA (25) | CD24 | 0.7049 | 0.0000 | 0.0002 | 0.0043 |
| PCSA (3) | BCNP | 0.7028 | 0.0000 | 0.0002 | 0.0052 |
| PCSA (25) | HSP70 | 0.7027 | 0.0000 | 0.0002 | 0.0053 |
| PCSA (25) 43 | MMP9 | 0.7022 | 0.0000 | 0.0002 | 0.0056 |

The analysis for the results in Table 33 was high-risk of PCa vs. low-risk of PCa samples. High risk is defined as postive cancer biopsy as well as HGPIN and ATYPIA/ASAP. Low risk samples are the remainder.

TABLE 33
High-risk of PCa vs. Low-risk of PCa

| | | Effect | Wilcoxon | | |
|---|---|---|---|---|---|
| Detector | Capture | size | p-value | FDR | Bonf |
| Epcam | MMP7 | 0.8269 | 0.0000 | 0.0000 | 0.0000 |
| Epcam | BCNP | 0.7399 | 0.0000 | 0.0000 | 0.0000 |
| PCSA (25) | MMP7 | 0.7284 | 0.0000 | 0.0001 | 0.0002 |
| PCSA (25) | KLK2 | 0.7222 | 0.0000 | 0.0001 | 0.0004 |
| PCSA (25) | SPDEF | 0.7025 | 0.0000 | 0.0006 | 0.0031 |
| PCSA (25) | ADAM10 | 0.6988 | 0.0000 | 0.0007 | 0.0046 |
| CD81 | MMP7 | 0.6982 | 0.0000 | 0.0007 | 0.0049 |
| PCSA (25) | SSX2 | 0.6929 | 0.0000 | 0.0010 | 0.0083 |
| PCSA (25) | PBP | 0.6925 | 0.0000 | 0.0010 | 0.0087 |
| PCSA (25) | EpCAM | 0.6914 | 0.0000 | 0.0010 | 0.0096 |
| PCSA (25) | p53 | 0.6857 | 0.0000 | 0.0015 | 0.0169 |
| Muc2 | MMP7 | 0.6847 | 0.0000 | 0.0015 | 0.0186 |
| Muc2 | PRL | 0.6845 | 0.0000 | 0.0015 | 0.0190 |
| PCSA (25) | CD24 | 0.6828 | 0.0001 | 0.0016 | 0.0223 |
| PCSA (25) | MMP9 | 0.6818 | 0.0001 | 0.0016 | 0.0245 |
| PCSA (25) | EGFR | 0.6781 | 0.0001 | 0.0022 | 0.0344 |
| PCSA (25) | IL-1B | 0.6767 | 0.0001 | 0.0023 | 0.0394 |
| PCSA (25) | CD9 | 0.6735 | 0.0001 | 0.0029 | 0.0527 |
| PCSA (25) | SSX4 | 0.6720 | 0.0001 | 0.0030 | 0.0600 |
| MFGE8 | TIMP-1 | 0.6759 | 0.0001 | 0.0030 | 0.0604 |
| PCSA (25) | HER3 (ErbB3) | 0.6713 | 0.0001 | 0.0031 | 0.0641 |
| MFGE8 | MMP7 | 0.6740 | 0.0002 | 0.0032 | 0.0714 |
| PCSA (25) | HSP70 | 0.6613 | 0.0004 | 0.0067 | 0.1534 |
| PCSA (25) | CYFRA21-1 | 0.6600 | 0.0004 | 0.0070 | 0.1713 |
| MFGE8 | BCNP | 0.6633 | 0.0004 | 0.0070 | 0.1761 |

The analysis for the results in Table 34 consisted of all PCA+ samples compared to inflammation positive samples. All other outcomes were excluded.

TABLE 34
Prostate Cancer v Prostate Inflammatory Conditions

| | | Effect | Wilcoxon | | |
|---|---|---|---|---|---|
| Detector | Capture | size | p-value | FDR | Bonf |
| EpCam | MMP7 | 0.8196 | 0.0000 | 0.0007 | 0.0007 |
| EpCam | BCNP | 0.7914 | 0.0000 | 0.0026 | 0.0052 |
| PCSA (25) | IL-1B | 0.7579 | 0.0001 | 0.0130 | 0.0470 |
| PCSA (25) | ADAM10 | 0.7562 | 0.0001 | 0.0130 | 0.0520 |
| PCSA (25) | KLK2 | 0.7319 | 0.0005 | 0.0386 | 0.2177 |
| PCSA (25) | EGFR | 0.7308 | 0.0005 | 0.0386 | 0.2313 |
| PCSA (25) | SPDEF | 0.7256 | 0.0007 | 0.0439 | 0.3076 |
| PCSA (25) | CD9 | 0.7232 | 0.0008 | 0.0439 | 0.3513 |
| MFGE8 | TIMP-1 | 0.7168 | 0.0013 | 0.0619 | 0.5574 |
| PCSA (25) | p53 | 0.7048 | 0.0021 | 0.0921 | 0.9213 |
| PCSA (25) | MMP7 | 0.7021 | 0.0024 | 0.0959 | 1.0000 |
| PCSA (25) | PBP | 0.6966 | 0.0032 | 0.1148 | 1.0000 |

The analysis for the results in Table 35 consisted of all PCA+ samples compared to “benign” prostate conditions, where “benign” is defined as a negative biopsy without inflammatory condition.

TABLE 35
Prostate Cancer v Non-inflammatory Benign Prostate Conditions

| | | Effect | Wilcoxon | | |
|---|---|---|---|---|---|
| Detector | Capture | size | p-value | FDR | Bonf |
| Epcam | MMP7 | 0.9161 | 0.0000 | 0.0000 | 0.0000 |
| PCSA (25) | MMP7 | 0.8465 | 0.0000 | 0.0000 | 0.0000 |
| Epcam | BCNP | 0.7964 | 0.0000 | 0.0000 | 0.0000 |
| PCSA (25) | KLK2 | 0.7864 | 0.0000 | 0.0000 | 0.0001 |
| CD81 | MMP7 | 0.7714 | 0.0000 | 0.0000 | 0.0002 |
| PCSA (25) | SPDEF | 0.7679 | 0.0000 | 0.0001 | 0.0003 |
| PCSA (25) | EpCAM | 0.7648 | 0.0000 | 0.0001 | 0.0004 |
| PCSA (25) | SSX2 | 0.7544 | 0.0000 | 0.0001 | 0.0012 |
| PCSA (25) | ADAM10 | 0.7541 | 0.0000 | 0.0001 | 0.0012 |
| MFGE8 | MMP7 | 0.7605 | 0.0000 | 0.0001 | 0.0013 |
| PCSA (25) | PBP | 0.7474 | 0.0000 | 0.0002 | 0.0022 |
| PCSA (25) | SSX4 | 0.7441 | 0.0000 | 0.0002 | 0.0029 |
| Muc2 | MMP7 | 0.7430 | 0.0000 | 0.0002 | 0.0032 |
| PCSA (25) | p53 | 0.7391 | 0.0000 | 0.0003 | 0.0045 |
| PCSA (25) | EGFR | 0.7344 | 0.0000 | 0.0004 | 0.0067 |
| PCSA (25) | CD24 | 0.7344 | 0.0000 | 0.0004 | 0.0067 |
| PCSA (25) | MMP9 | 0.7324 | 0.0000 | 0.0005 | 0.0079 |
| PCSA (25) | SERPINB3 | 0.7295 | 0.0000 | 0.0005 | 0.0101 |
| PCSA (25) | HSP70 | 0.7295 | 0.0000 | 0.0005 | 0.0101 |
| PCSA (25) | CD3 | 0.7256 | 0.0000 | 0.0007 | 0.0137 |
| PCSA (25) | IL-1B | 0.7245 | 0.0000 | 0.0007 | 0.0151 |
| PCSA (25) | CD9 | 0.7221 | 0.0000 | 0.0008 | 0.0182 |
| PCSA (25) | HER3 (ErbB3) | 0.7198 | 0.0001 | 0.0010 | 0.0220 |
| PCSA (25) | TIMP | 0.7171 | 0.0001 | 0.0011 | 0.0270 |
| PCSA (25) | CYFRA21-1 | 0.7163 | 0.0001 | 0.0012 | 0.0289 |

Table 36 shows the results of comparing all PCA+ samples with all high-grade prostatic intraepithelial neoplasia (HGPIN) samples.

TABLE 36
Prostate Cancer v HGPIN

| | | Effect | Wilcoxon | | |
|---|---|---|---|---|---|
| Detector | Capture | size | p-value | FDR | Bonf |
| Epcam | MMP7 | 0.7945 | 0.0000 | 0.0074 | 0.0143 |
| PCSA (25) | MMP7 | 0.7939 | 0.0000 | 0.0074 | 0.0148 |
| PCSA (25) | ADAM 10 | 0.7727 | 0.0001 | 0.0174 | 0.0523 |
| PCSA (25) | IL-1B | 0.7644 | 0.0002 | 0.0210 | 0.0840 |
| Epcam | BCNP | 0.7484 | 0.0005 | 0.0329 | 0.2009 |
| PCSA (25) | EGFR | 0.7458 | 0.0005 | 0.0329 | 0.2300 |
| PCSA (3) | BCNP | 0.7458 | 0.0005 | 0.0329 | 0.2300 |
| PCSA (25) | CD9 | 0.7298 | 0.0012 | 0.0651 | 0.5206 |
| PCSA (25) | SPDEF | 0.7273 | 0.0014 | 0.0656 | 0.5906 |
| Epcam | TRAIL R2 | 0.7209 | 0.0018 | 0.0732 | 0.8055 |
| PCSA (25) | AURKB | 0.7209 | 0.0018 | 0.0732 | 0.8055 |
| PCSA (25) | SERPINB3 | 0.7164 | 0.0023 | 0.0802 | 0.9963 |
| Epcam | NGAL | 0.7154 | 0.0024 | 0.0802 | 1.0000 |
| PCSA (25) | seprase/FAP | 0.7113 | 0.0029 | 0.0837 | 1.0000 |
| PCSA (25) | KLK2 | 0.7113 | 0.0029 | 0.0837 | 1.0000 |
| PCSA (25) | ERG | 0.7100 | 0.0031 | 0.0837 | 1.0000 |
| PCSA (25) | TRAIL R2 | 0.7087 | 0.0033 | 0.0837 | 1.0000 |
| PCSA (25) | STEAP | 0.7068 | 0.0036 | 0.0862 | 1.0000 |
| PCSA (25) | EpCAM | 0.6997 | 0.0049 | 0.0983 | 1.0000 |
| CD81 | MMP7 | 0.6991 | 0.0050 | 0.0983 | 1.0000 |
| MFGE8 | MMP7 | 0.7042 | 0.0051 | 0.0983 | 1.0000 |
| PCSA (25) | TGM2 | 0.6978 | 0.0053 | 0.0983 | 1.0000 |
| PCSA (25) | CRP | 0.6972 | 0.0054 | 0.0983 | 1.0000 |
| PCSA (25) | CD81 | 0.6959 | 0.0057 | 0.0983 | 1.0000 |
| PCSA (25) | p53 | 0.6959 | 0.0057 | 0.0983 | 1.0000 |

The results in Table 37 were obtained by comparing bins of total Gleason score for subjects with cancer biopsy. Samples were grouped by low Gleason (<5), intermediate Gleason (6-9) and high Gleason (>10). P-values were not corrected due to small sample sizes.

TABLE 37
Gleason Score Comparison

| | | Effect | KW p- |
|---|---|---|---|
| Detector | Capture | size | value |
| CD81 | CD41 | 8.1729 | 0.0043 |
| CD81 | VCAN | 7.3313 | 0.0068 |
| CD81 | MUC1 | 7.1483 | 0.0075 |
| CD81 | Integrin | 7.0934 | 0.0077 |
| Epcam | EpCAM | 6.8867 | 0.0087 |
| CD81 | Gro alpha | 6.8058 | 0.0091 |
| CD81 | PIM1 | 6.4976 | 0.0108 |
| CD81 | GM-CSF | 6.4846 | 0.0109 |
| CD81 | TRAIL R2 | 6.3732 | 0.0116 |
| CD81 | RUNX2 | 5.9838 | 0.0144 |
| CD81 | EpCAM | 5.8523 | 0.0156 |
| CD81 | PSMA | 5.8309 | 0.0157 |
| CD81 | TWEAK | 5.7151 | 0.0168 |
| CD81 | EphA2 | 5.6480 | 0.0175 |
| CD81 | CD24 | 5.5969 | 0.0180 |
| CD81 | S100-A4 | 5.5323 | 0.0187 |
| CD81 | SPC | 5.4956 | 0.0191 |
| Epcam | EphA2 | 5.4743 | 0.0193 |
| CD81 | AURKB | 5.4580 | 0.0195 |
| CD81 | IL-1B | 5.4358 | 0.0197 |
| CD81 | ERG | 5.3871 | 0.0203 |
| CD81 | EGFR | 5.2719 | 0.0217 |
| CD81 | ADAM10 | 5.2376 | 0.0221 |

In Table 38, results were obtained by comparing groups of samples in the following categories: 1) benign; 2) inflammation; 3) ATYPIA/ASAP/HGPIN; 4) PCA+, total Gleason score=6-9.

TABLE 38
Clinical Category Comparison

| | | Effect | KW p- | | |
|---|---|---|---|---|---|
| Detector | Capture | size | value | FDR | Bonf |
| Epcam | MMP7 | 52.6024 | 0.0000 | 0.0000 | 0.0000 |
| PCSA (25) | MMP7 | 34.1291 | 0.0000 | 0.0000 | 0.0000 |
| Epcam | BCNP | 31.4758 | 0.0000 | 0.0000 | 0.0000 |
| CD81 | MMP7 | 26.3295 | 0.0000 | 0.0000 | 0.0001 |
| PCSA (25) | ADAM 10 | 25.7130 | 0.0000 | 0.0000 | 0.0002 |
| PCSA (25) | EpCAM | 25.2041 | 0.0000 | 0.0000 | 0.0002 |
| PCSA (25) | SPDEF | 25.1335 | 0.0000 | 0.0000 | 0.0002 |
| PCSA (25) | IL-1B | 23.5724 | 0.0000 | 0.0001 | 0.0005 |
| PCSA (25) | PBP | 22.2470 | 0.0000 | 0.0001 | 0.0010 |
| PCSA (25) | EGFR | 21.0674 | 0.0000 | 0.0002 | 0.0019 |
| PCSA (25) | SSX4 | 20.1831 | 0.0000 | 0.0003 | 0.0031 |
| PCSA (25) | SSX2 | 19.4117 | 0.0000 | 0.0004 | 0.0046 |
| PCSA (25) | P53 | 19.0491 | 0.0000 | 0.0004 | 0.0056 |
| PCSA (25) | KLK2 | 18.8417 | 0.0000 | 0.0004 | 0.0062 |
| PCSA (25) | MMP9 | 18.3870 | 0.0000 | 0.0005 | 0.0079 |
| PCSA (25) | CD9 | 18.1835 | 0.0000 | 0.0005 | 0.0088 |
| PCSA (25) | SERPINB3 | 17.5771 | 0.0000 | 0.0007 | 0.0121 |
| PCSA (25) | HSP70 | 17.2052 | 0.0000 | 0.0008 | 0.0147 |
| Epcam | p53 | 16.1627 | 0.0001 | 0.0013 | 0.0254 |
| PCSA (25) | CSA | 15.8084 | 0.0001 | 0.0015 | 0.0306 |
| PCSA (25) | HER3 (ErbB3) | 15.5570 | 0.0001 | 0.0016 | 0.0350 |
| Epcam | EpCAM | 15.5062 | 0.0001 | 0.0016 | 0.0359 |
| MFGE8 47 | MMP7 | 15.4614 | 0.0001 | 0.0016 | 0.0368 |
| PCSA (25) 34 | CD24 | 15.1291 | 0.0001 | 0.0018 | 0.0439 |
| PCSA (25) 53 | CYFRA21-1 | 14.9992 | 0.0001 | 0.0019 | 0.0470 |

In Table 39, results are shown for analysis of PCa+ subjects with total Gleason score ≧7 compared to PCa+ subjects with Gleason score of 6 and PCa− subjects.

TABLE 39
High Gleason v Others

| | | Effect | Wilcoxon | | |
|---|---|---|---|---|---|
| Detector | Capture | size | p-value | FDR | Bonf |
| Epcam | EpCAM | 0.7697 | 0.0000 | 0.0004 | 0.0010 |
| Epcam | MMP7 | 0.7688 | 0.0000 | 0.0004 | 0.0010 |
| Epcam | BCNP | 0.7660 | 0.0000 | 0.0004 | 0.0013 |
| Epcam | EGFR | 0.7509 | 0.0000 | 0.0012 | 0.0046 |
| Epcam | TGM2 | 0.7377 | 0.0000 | 0.0026 | 0.0131 |
| Epcam | CD9 | 0.7285 | 0.0001 | 0.0044 | 0.0264 |
| CD81 | MMP7 | 0.7264 | 0.0001 | 0.0044 | 0.0308 |
| Epcam | Integrin | 0.7203 | 0.0001 | 0.0051 | 0.0481 |
| Epcam | PBP | 0.7201 | 0.0001 | 0.0051 | 0.0486 |
| CD81 | BCNP | 0.7194 | 0.0001 | 0.0051 | 0.0510 |
| Epcam | p53 | 0.7150 | 0.0002 | 0.0063 | 0.0698 |
| Epcam | ADAM10 | 0.7138 | 0.0002 | 0.0064 | 0.0763 |
| Epcam | MUC1 | 0.7106 | 0.0002 | 0.0073 | 0.0949 |
| Epcam | CD41 | 0.7074 | 0.0003 | 0.0085 | 0.1185 |
| PCSA (25) | MS4A1 | 0.7058 | 0.0003 | 0.0088 | 0.1323 |
| PCSA (25) | MMP7 | 0.7028 | 0.0004 | 0.0095 | 0.1621 |
| Epcam | TRAIL R2 | 0.7007 | 0.0004 | 0.0095 | 0.1862 |
| Epcam | PSA | 0.6993 | 0.0005 | 0.0095 | 0.2041 |
| Epcam | hVEGFR2 | 0.6993 | 0.0005 | 0.0095 | 0.2041 |
| Epcam | CSA | 0.6986 | 0.0005 | 0.0095 | 0.2136 |
| Epcam | CD3 | 0.6983 | 0.0005 | 0.0095 | 0.2185 |
| PCSA (25) | ADAM10 | 0.6981 | 0.0005 | 0.0095 | 0.2202 |
| CD81 | PIM1 | 0.6979 | 0.0005 | 0.0095 | 0.2235 |
| Epcam | EphA2 | 0.6976 | 0.0005 | 0.0095 | 0.2287 |
| Epcam | DCRN | 0.6968 | 0.0006 | 0.0096 | 0.2411 |

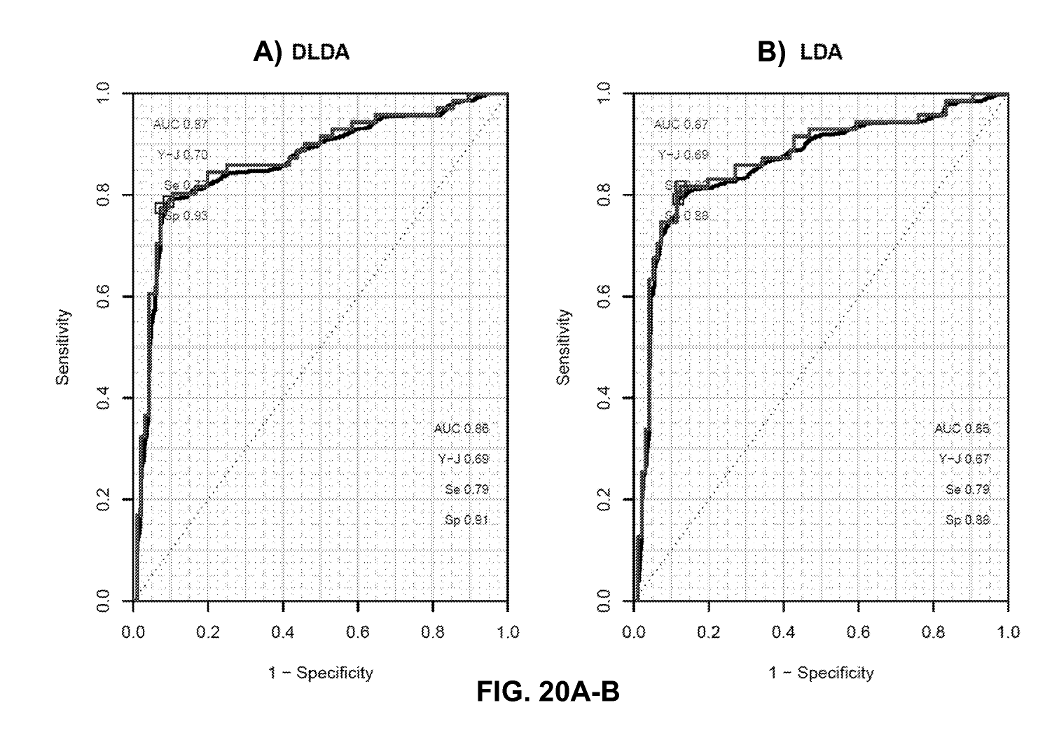

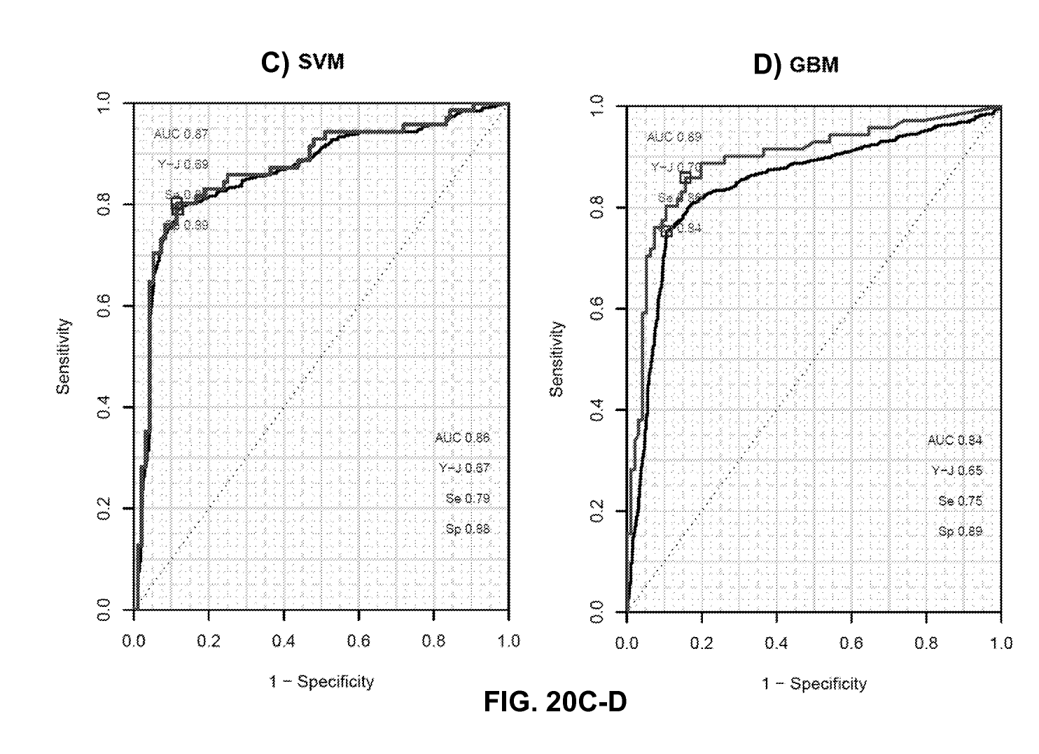

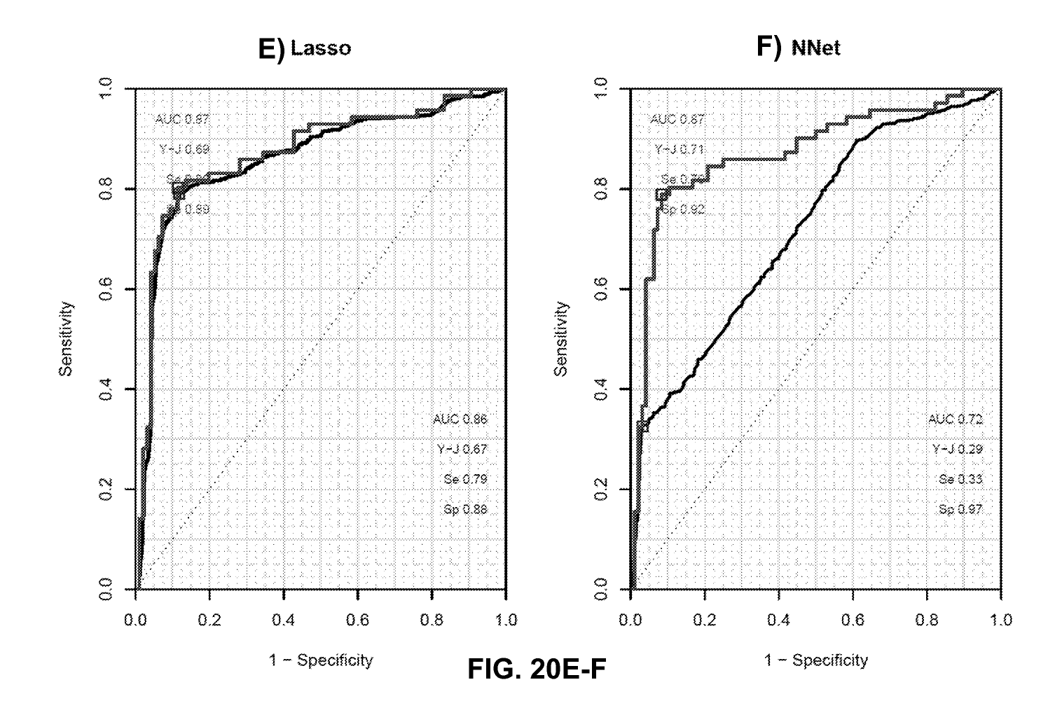

Multi-biomarker panels were constructed from the capture/detector agents in Table 28 on the plasma samples from patients in Table 26. Different multi-analyte class prediction models were compared, including linear discriminant analysis, diagonal linear discriminant analysis, shrunken centroids discriminant analysis, support vector machines, tree-based gradient boosting, lasso and neural network. Panels included 3-marker, 5-marker, 10-marker, 20-marker and 50-markers, where each “marker” refers to a capture-detector pair, such as MMP7 capture-PCSA detector and the like (see Table 28 for all pairs tested). Illustrative results for distinguishing prostate cancer (PCa+) samples from all other samples (PCA−) (see Table 26) using 3-marker combinations are shown in FIGS. 20A-F. In these figures, the dark grey line (more jagged line to the left) corresponds to resubstitution performance and the smoother black line was generated using 10-fold cross-validation. ROC curves are shown generated using diagonal linear discriminant analysis (FIG. 20A; resubstitution AUC=0.87; cross validation AUC=0.86), linear discriminant analysis (FIG. 20B; resubstitution AUC=0.87; cross validation AUC=0.86), support vector machine (FIG. 20C; resubstitution AUC=0.87; cross validation AUC=0.86), tree-based gradient boosting (FIG. 20D; resubstitution AUC=0.89; cross validation AUC=0.84), lasso (FIG. 20E; resubstitution AUC=0.87; cross validation AUC=0.86), and neural network (FIG. 20F; resubstitution AUC=0.87; cross validation AUC=0.72).

Illustrative 3-marker combinations, 5-marker combinations, and 10-marker combinations are shown in Table 40. Table 40 also shows the performance of the models using linear discrimant models in two different settings. Performance is shown as sensitivity and specificity at different threshold values. Results for “All samples” are from a comparison of prostate cancer samples versus all other patient samples. See Table 28 for individual marker combinations. Results for the “Restricted” sample cohort consisted of prostate cancer samples versus all other patients, wherein the cohort was constrained using the following criteria: PSA<10 μg/ml; Age<75; First biopsy cancers. See Table 40 for individual marker combinations. As seen in the table, the threshold can be adjusted to favor sensitivity or specificity as desired for the intended use.

TABLE 40
Multiple-marker Panels

| | Detector/Capture | | Linear Discriminant Analysis | |
|---|---|---|---|---|
| Model | Agents | | Sensitivity/Specificity | |
| size/identifier | Detector | Capture | All samples | Restricted |
| 3-marker | EpCam | MMP7 | 90/50 | 95/52 |
| | PCSA | MMP7 | 86/65 | 90/65 |
| | EpCam | BCNP | 82/70 | 82/80 |
| | | | | 80/88 |
| 5-marker | EpCam | MMP7 | 92/50 | 92/60 |
| | PCSA | MMP7 | 84/70 | 90/70 |
| | EpCam | BCNP | 80/77 | 85/78 |
| | PCSA | ADAM10 | | 80/81 |
| | PCSA | KLK2 | | |
| 10-marker | EpCam | MMP7 | 92/50 | 95/53 |
| | PCSA | MMP7 | 84/70 | 90/65 |
| | EpCam | BCNP | 80/75 | 85/80 |
| | PCSA | ADAM10 | | 80/82 |
| | PCSA | KLK2 | | |
| | PCSA | SPDEF | | |
| | CD81 | MMP7 | | |
| | PCSA | EpCam | | |
| | MFGE8 | MMP7 | | |
| | PCSA | IL-8 | | |

Results of optimal marker panels for various settings are shown in Table 41. Linear discriminant analysis is shown. In the table, “Model A” refers to the complete sample set (see Table 29), “Model B” refers to the restricted sample set (see Table 30), and “Model C” refers to the restricted cohort without watchful waiting samples but with previous negative biopsy (see Table 31).

TABLE 41
Type and Performance of Various Models

| | | Patient Set | |
|---|---|---|---|
| | | All Samples (N = 175) | Restricted (N = 127) |
| Intend- | Optimized | 5-marker linear Model A | 3-marker linear Model B |
| ed | Accuracy | AUC = 0.87 | AUC = 0.90 |
| Use | | Sensitivity = 82 | Sensitivity = 90 |
| | | Specificity = 80 | Specificity = 80 |
| | Optimized | 5-marker linear Model A | 5-marker linear Model C |
| | Sensitivity | AUC = 0.87 | AUC = 0.89 |
| | | Sensitivity = 92 | Sensitivity = 95 |
| | | Specificity = 50 | Specificity = 60 |

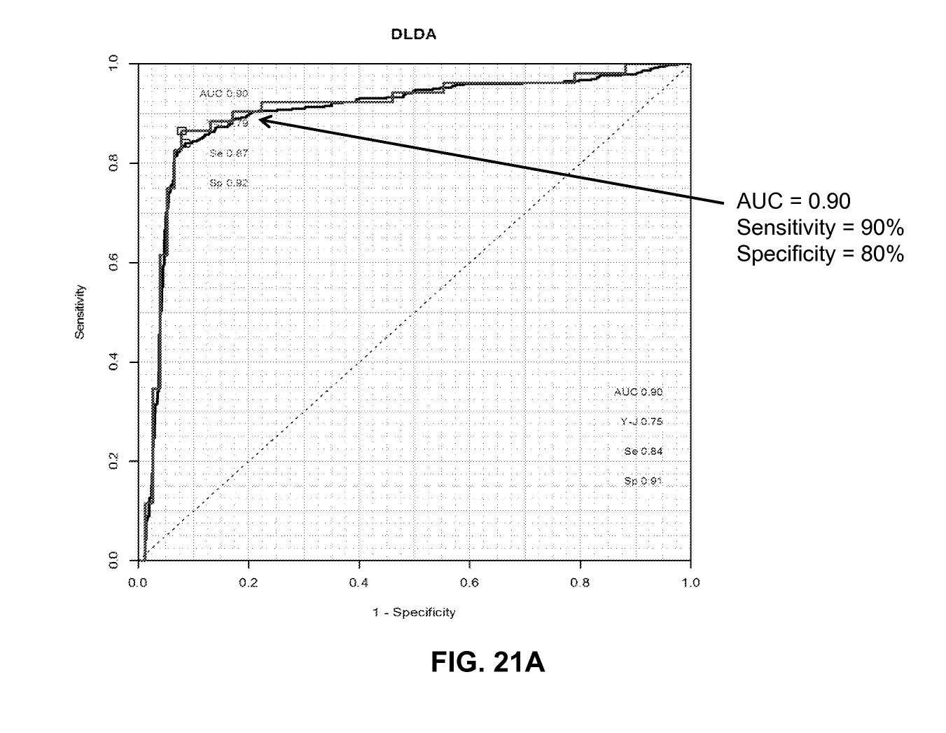

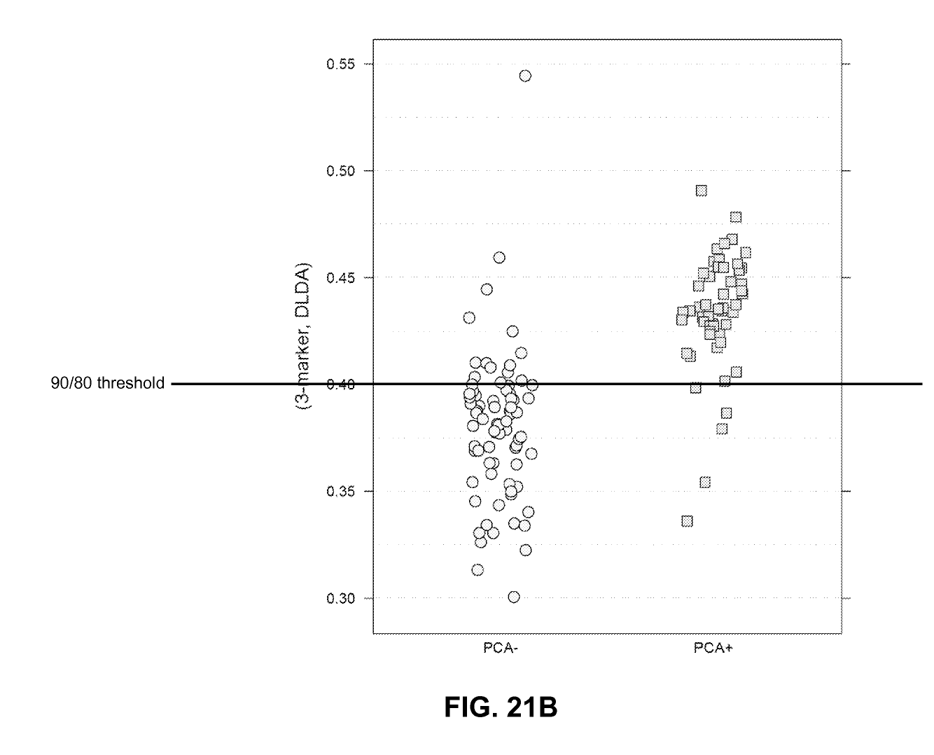

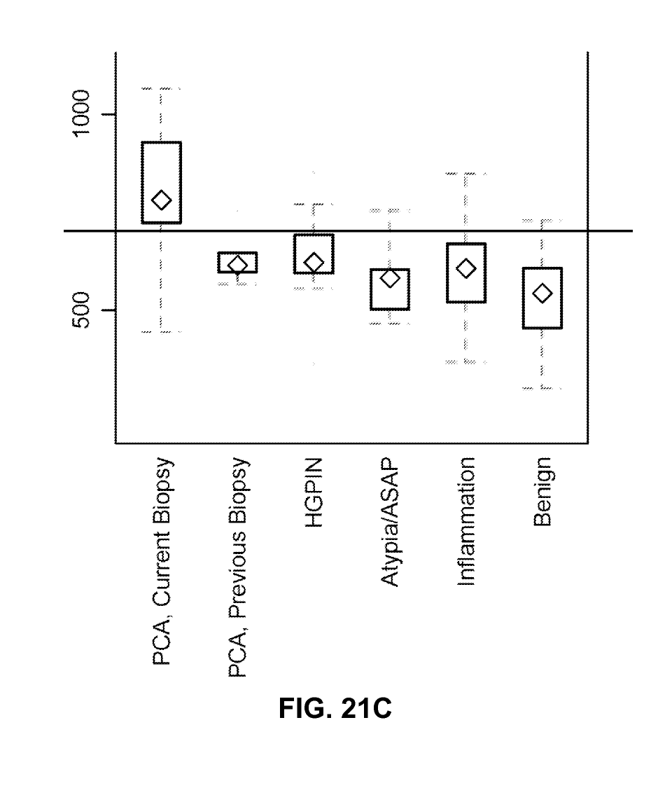

The Model B three marker panel consisted of the following markers: 1) Epcam detector-MMP7 capture; 2) PCSA detector-MMP7 capture; 3) Epcam detector-BCNP capture. An ROC curve generated using a diagonal linear discriminant analysis in this setting is shown in FIG. 21A. In the figure, the arrow indicates the threshold point along the curve where sensitivity equals 90% and specificity equals 80%. Another view of this threshold is shown in FIG. 21B, which shows the distribution of PCA+ and PCA− samples falling on either side of the indicated threshold line. The individual contribution of the Epcam detector-MMP7 capture marker is shown in FIG. 21C. “PCA, Current Biopsy” refers to men who had a first positive biopsy, whereas “PCA, Previous Biopsy” refers to the watchful waiting cohort. The figure shows good separation of the PCA+ first biopsy samples from all other samples using only this marker set.

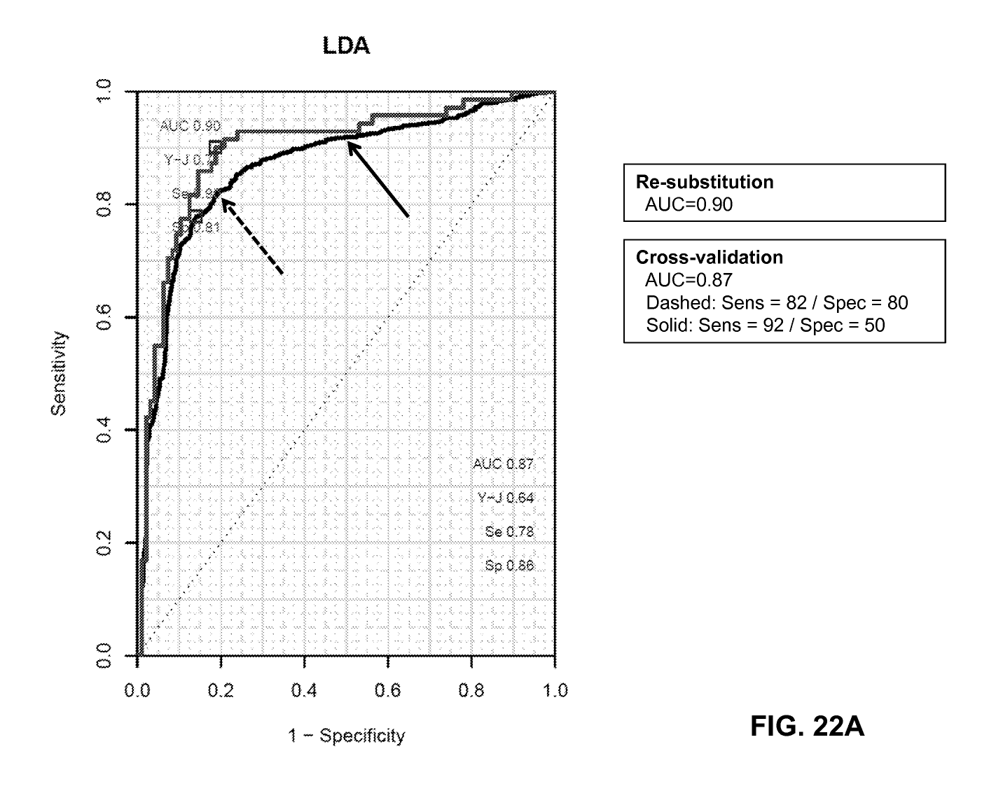

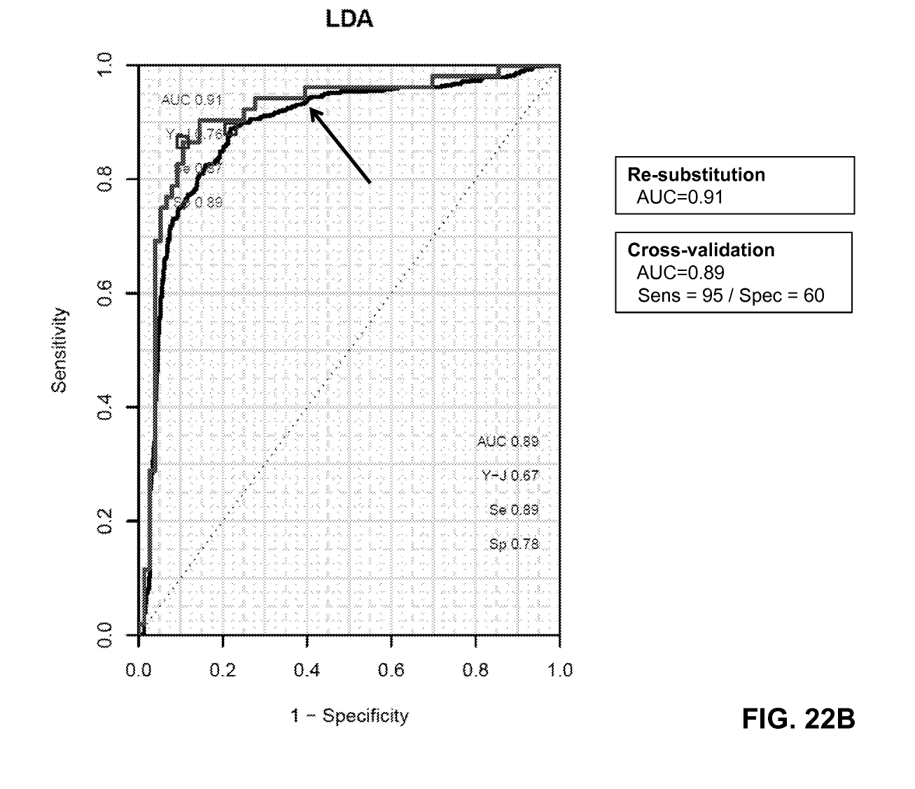

The performance of the 5-marker panel was also determined in the Model A and Model C settings using a linear discriminant analysis. In both settings, AUC was calculated using 10-fold cross-validation or re-substitution methodology. ROC curves for the Model A setting (i.e., all PCa versus all other patient samples) are shown in FIG. 22A. The marker panel in this setting consisted of: 1) Epcam detector-MMP7 capture; 2) PCSA detector-MMP7 capture; 3) Epcam detector-BCNP capture; 4) PCSA detector-Adam10 capture; and 5) PCSA detector-KLK2 capture. In FIG. 22A, the upper more jagged line corresponds to the re-substitution method. The AUC was 0.90. Using cross-validation, the calculated AUC was 0.87. At the point indicated by the solid arrow, the model using cross-validation achieved 92% sensitivity and 50% specificity. At the point indicated by the solid arrow, the model using cross-validation achieved 82% sensitivity and 80% specificity. ROC curves for the Model C setting (i.e., restricted sample set as described above for Table 30) are shown in FIG. 22B. The marker panel in this setting consisted of: 1) Epcam detector-MMP7 capture; 2) PCSA detector-MMP7 capture; 3) Epcam detector-BCNP capture; 4) PCSA detector-Adam10 capture; and 5) CD81 detector-MMP7 capture. In FIG. 22B, the upper more jagged line corresponds to the re-substitution method. The AUC was 0.91. Using cross-validation, the calculated AUC was 0.89. At the point indicated by the arrow, the cross-validation model achieved 95% sensitivity and 60% specificity.

In all settings, the cMV approach was much more accurate than serum PSA testing, which only had an AUC of about 0.60 in these sample cohorts.

### Example 38

### Microfluidic Detection of microRNAs

In this Example, a microfluidic system is used to detect microRNAs using quantitative PCR (qPCR). The starting sample can be microRNAs isolated from a biological sample such as blood, serum or plasma, or from concentrated microvesicles from these or other biological samples. Methods to extract microRNAs are described above or known in the art. In this Example, the Fluidigm BioMark™ System is used (Fluidigm Corporation, South San Francisco, Calif.). The microfluidic system can be used to perform multiplex analysis of miRs (i.e., assay multiple miRs in a single assay run).

Reverse Transcription (RT) of samples—use layout form specific to Fluidigm when performing multiplex reactions:
1. Creation of 20× Multiplex RT pools from individual assays:
   A. Aliquot desired volume of each individual 5×RT primer into a 1.7 ml microcentrifuge tube. Use primers that can be multiplexed together as appropriate.
   B. Make 50 μl aliquots of the RT primer pool and completely dry them down in a speed vacuum at 45° C.
   C. Resuspend the primer pool aliquots in 25% of the individual assay input volume with nuclease free ddH2O (i.e. if 100 μl of each 5× primer was added to the primer pool then resuspend in a final volume of 25 μl). This is now the 20× multiplex RT pool.
2. Reverse Transcription
   A. Create RT plate layout.
   B. From −20° C. freezer, take out 10×RT buffer, 100 mMdNTP mix, Rnase inhibitor, Multiscribe RT enzyme, from −80° C. RNA sample(s), set all on ice.
   C. In the pre-amp hood make up Master Mix for 7.5 μl total RT reaction volume per sample, for both the singleplex and multiplex reactions, by mixing the RT reagents in the order and amount specified in the RT experiment sheet found in the location listed above.
   D. Aliquot the specified volume of RT master mix for singleplex and multiplex reactions into a 96 well PCR plate.
   E. Add the specified RNA input volume for singleplex and multiplex reactions into the appropriate wells containing your aliquoted RT master mix.
   F. Seal the PCR plate with a PCR seal.
   G. Centrifuge plate at 2000 rpm for 30 seconds.
   H. Set up a thermal cycler with the miRNA RT protocol—make sure the program is set to the correct cycling parameters (as seen on RT layout sheet) and reaction volume is set to 10 μl.
   I. Add plate to the machine and start the program (takes about 1 hr 5 minutes if the machine is warm).

Pre-amplification (PreAmp) of samples—use layout form specific to BioMark:
1. Creation of 0.2× Multiplex miR Assay Pool:
   A. Add desired volume in equal amounts of each individual 20×miR assay into a 1.7 ml microcentrifuge tube.
   B. If n=number of assays in the multiplex pool, add n μl of the pooled 20×miR assays to 100-n μl of DNA suspension buffer.
2. Creation of 0.2× singleplex miR assay
   A. Dilute each individual miR assay 1:100 with DNA suspension buffer.
3. PreAmp
   A. Create PreAmp plate layout.
   B. From −4° C. fridge, take out Taqman PreAmp Master Mix.
   C. In the pre-amp hood make up the master mix for 10 μl total singleplex PreAmp reaction volume per sample, and 5 μl total multiplex PreAmp reaction volume per sample by mixing the PreAmp reagents in the order and amount specified in the PreAmp experiment sheet found in the location listed above.
   D. Aliquot the specified volume of PreAmp master mix for singleplex and multiplex reactions into a 96 well PCR plate
   E. Add the specified volume of sample cDNA for singleplex and multiplex reactions into the appropriate wells containing aliquoted PreAmp master mix.
   F. Seal the PCR plate with a PCR seal.
   G. Centrifuge plate at 2000 rpm for 30 seconds.
   H. Set up a thermal cycler with the miRNA PreAmp 12 cycles protocol-check to make sure that the program is set to the correct cycling parameters (as seen on the PreAmp layout sheet) and the reaction volume is set to 10 μl.
   I. Add plate to the machine and start the program (takes about 1 hr 10 minutes if the machine is warm).
   J. After completion of the PreAmp program, dilute the singleplex reactions 1:4 and multiplex reactions 1:5 with DNA suspension buffer.
   K. Samples can be stored at −20° C. for up to one week.

qPCR of samples—use layout form specific to BioMark:
1. Priming the 48.48 and 96.96 dynamic array IFC (integrated fluidic circuit) chips (Fluidigm)
   A. Remove the chip from its package and inject control line fluid into each of the 2 accumulator injection ports on the chip.
   *Use the chip within 24 hrs of opening the package
   *Due to different accumulator volume capacity, only use 48.48 syringes (300 μl of control line fluid) with 48.48 chips, and only use 96.96 syringes (150 μl of control line fluid) with 96.96 chips
   *Control line fluid on the chip or in the inlets makes the chip unusable
   *Load the chip within 60 minutes of priming
   B. Place the chip into the appropriate IFC controller (MX for 48.48 chip; HX for 96.96 chip), then run the Prime (113× for 48.48; 136× for 96.96) script to prime the control line fluid into the chip.
2. Preparing 10× Assays
   A. Create a qPCR plate layout.
   B. From the −20° C. freezer, take out 20× Taqman Assay and 2× Assay loading reagent.
   C. In the pre-amp hood make up 10× Assay mix for 5 μl total volume per chip inlet by mixing the 10× assay reagents in the amount specified in the qPCR experiment sheet found in the location listed above.
   Note: Adjust # Assay replicates field on the qPCR experiment sheet based on the # of replicate reactions desired for each sample. This will depend on the total number of assays and samples tested on a single chip since replicate reactions can be achieved by either adding replicates of a single assay to the assay inlet side of the chip, or by adding replicates of a single sample to the sample inlet side of the chip.
   D. All assay inlets must have assay loading reagent. Prepare enough assay loading reagent and water, in a 1:1 ratio, to fill all unused assay inlets with 5 μl each.
3. Preparing Sample Pre-Mix and Samples
   A. From the −4° C. fridge take out 2×ABI Taqman Universal PCR Master Mix, and from the −20° freezer take out the 20×GE Sample Loading Reagent.
   B. In the pre-amp hood make up enough Sample Pre-Mix to fill an entire chip by mixing the sample pre-mix reagents in the amount specified in the qPCR experiment sheet found in the location listed above.
   C. Aliquot 4.4 μl of Sample Pre-Mix into enough wells of a 96 well PCR plate in order to fill an entire chip (48 or 96).
   D. In the post-amp room add 3.6 μl of diluted PreAmp samples to the appropriate wells of the previously aliquoted 4.4 μl of Sample Pre-Mix.
   E. All sample inlets must have sample loading reagent. For unused sample inlets be sure to add 3.6 μl of water to the previously aliquoted 4.4 μl of Sample Pre-Mix.
4. Loading the Chip
   Vortex thoroughly and centrifuge all assay and sample solutions before pipetting into the chip inlets. Failure to do so may result in a decrease in data quality.
   While pipetting, avoid going past the first stop on the pipette. Doing so may introduce bubbles into the inlets.
   A. When the Prime (113× for 48.48; 136× for 96.96) script has finished, remove the primed chip from the IFC Controller and pipette 5 μl of each assay and each sample into their respective inlets on the chip.
   B. Return the chip to the IFC Controller.
   C. Using the IFC Controller software, run the Load Mix (113× for 48.48; 136× for 96.96) script to load the samples and assays into the chip.
   D. When the Load Mix (113× for 48.48; 136× for 96.96) script has finished, remove the loaded chip from the IFC Controller.
   E. Use clear tape to remove any dust particles from the chip surface.
   F. Remove and discard the blue protective film from the bottom of the chip.
   G. The chip is now ready to run. Start the chip run on the instrument immediately after loading the chip.
5. Using the Data Collection Software
   A. Double-click the Data Collection Software icon on the desktop to launch the software.
   B. Click Start a New Run.
   C. Check the status bar to verify that the camera and lamp are ready. Make sure that both are green before proceeding.
   *Note (when running a 96.96 chip, it is not necessary to have the lamp fully warmed up before proceeding. For the 96.96 chip only, there is a thermal mix step prior to the PCR cycling during which time the lamp will be able to fully warm up.)
   D. Place the chip into the reader with the A1 position matching up with the notched corner of the chip.
   E. Click Load.
   F. Verify the chip barcode and chip type.
      (1) Click Next.
   G. Chip Run file.
      (1) Select New.
      (2) Enter desired chip run name.
      (3) Click Next.
   H. Application, Reference, Probes.
      (1) Select Application Type-Gene Expression.
      (2) Select Passive Reference (ROX).
      (3) Select Assay-Single probe
      (4) Select probe types-FAM-MGB
      (5) Click Next.
   I. Click Browse to find thermal protocol file-No UNG Erase 96×96 (or 48×48) Standard.pc1.
   J. Confirm Auto Exposure is selected
   K. Click Next.
   L. Verify the chip run information.
      *Note (when using a No UNG Erase thermal protocol, the protocol title listed in the run information will still appear as GE 96×96 Standard v1.pc1.)
   M. Click Start Run.
   N. If you are running a 96.96 chip and the lamp is not fully warmed up you may choose to ignore the warning and start the run. As mentioned above, the thermal mix step doesn't require the lamp to be fully warmed up and will give it enough time to reach the required temperature.
   O. The 96.96 chip run time is about 2.25 hrs and the 48.48 chip run time is just under 2 hrs.

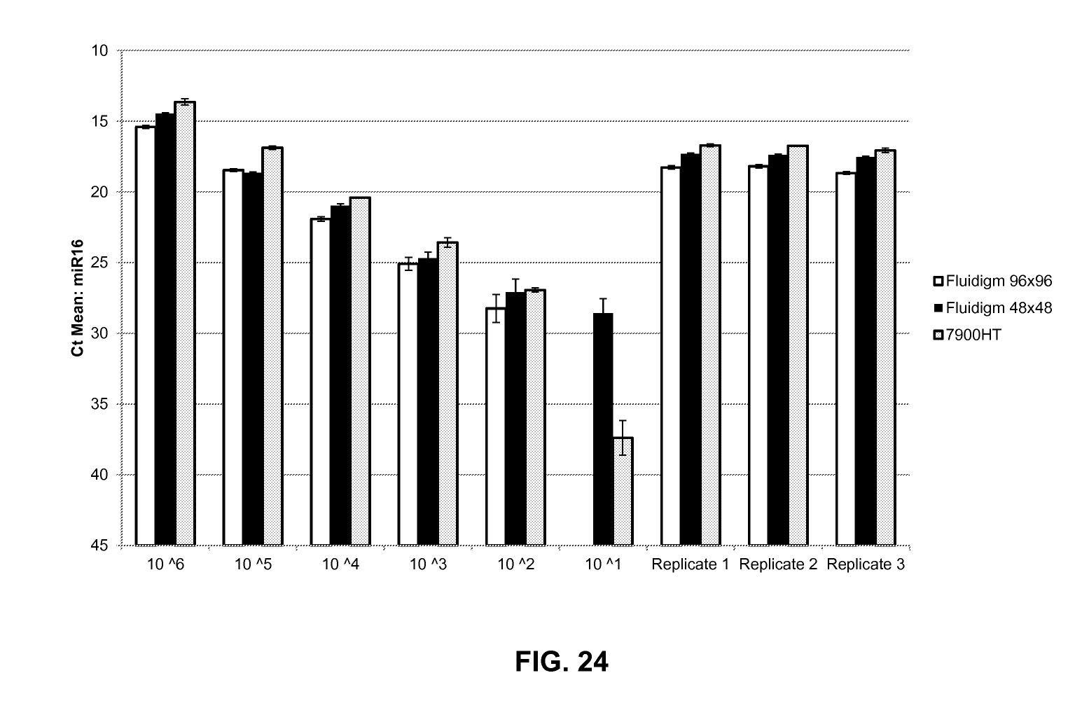

FIG. 24 shows detection of a standard curve for a synthetic miR16 standard (10̂6-10̂1) and detection of miR16 in triplicate from a human plasma sample. As indicated by the legend, the data was taken from a Fluidigm Biomark using 48.48 Dynamic Array™ IFCs, 96.96 Dynamic Array™ IFCs, or with an ABI 7900HT Taqman assay (Applied Biosystems, Foster City, Calif.). All levels were determined under multiplex conditions. Both systems and conditions showed similar performance.

### Example 39

### Comparison of Prostate Cancer (PCa) and Normal Control Profiles Using Antibody Arrays

In this Example, cMV were queried using antibody arrays to identify a cMV protein signature that distinguishes between normal control (i.e., no prostate cancer) and prostate cancer (PCa) patients, and patients with benign prostate conditions (BPH, HGPIN, inflammation). The sample set comprised plasma-derived cMVs from 18 PCa patients and from 10 patients from each of BPH, HGPIN and inflammation. The samples were incubated on a Full Moon BioSystems 649 antibody array (Full Moon BioSystems, Inc., Sunnyvale, Calif.) according to the manufacturer's instructions. Arrays were scanned on an Agilent scanner and data from images was extracted using Feature Extractor software (Agilent Technologies, Inc., Santa Clara, Calif.). Extracted data was normalized to array negative controls and normalized fluorescent values were analyzed with GeneSpring GX software (Agilent).

Fold change comparison of cMVs detected in the PCa samples versus the benign samples identified 18 markers elevated in prostate cancer with a fold-change greater than 1.5, as shown in Table 42. And 27 markers were identified whose expression was significantly different between PCa and the other diagnostic classes, as shown in Table 43. In Table 43, FC refers to fold change. As shown in this table, the greatest fold changes were observed between PCa and inflammation and HGPIN.

TABLE 42
cMV markers elevated in PCa over benign

| Protein | Fold change in cancer |
|---|---|
| Alkaline Phosphatase (AP) | 2.14 |
| CD63 | 1.93 |
| MyoD1 | 1.81 |
| Neuron Specific Enolase | 1.78 |
| MAP1B | 1.76 |
| CNPase | 1.72 |
| Prohibitin | 1.69 |
| CD45RO | 1.63 |
| Heat Shock Protein 27 | 1.60 |
| Collagen II | 1.60 |
| Laminin B1/b1 | 1.59 |
| Gail | 1.59 |
| CDw75 | 1.57 |
| bcl-XL | 1.57 |
| Laminin-s | 1.53 |
| Ferritin | 1.53 |
| CD21 | 1.51 |
| ADP-ribosylation Factor (ARF-6) | 1.51 |

TABLE 43
cMV markers statistically significantly different
between PCa and other diagnostic classes

| | Corrected | FC | FC | FC |
|---|---|---|---|---|
| Name | p-value | benign | inflammation | HGPIN |
| CD56/NCAM-1 | 0.014 | −1.41 | −3.28 | −5.42 |
| Heat Shock Protein | 0.024 | −1.60 | −3.24 | −5.33 |
| 27/hsp27 | | | | |
| CD45RO | 0.024 | −1.63 | −2.66 | −4.46 |
| MAP1B | 0.024 | −1.76 | −2.46 | −2.84 |
| MyoD1 | 0.024 | −1.81 | −3.15 | −4.95 |
| CD45/T200/LCA | 0.028 | −1.48 | −2.07 | −3.07 |
| CD3zeta | 0.028 | −1.42 | −3.08 | −3.51 |
| Laminin-s | 0.028 | −1.53 | −2.46 | −3.26 |
| bcl-XL | 0.028 | −1.57 | −2.40 | −3.45 |
| Rad18 | 0.028 | −1.19 | −2.16 | −2.52 |
| Gai1 | 0.032 | −1.59 | −1.99 | −3.16 |
| Thymidylate Synthase | 0.032 | −1.50 | −2.38 | −2.87 |
| Alkaline Phosphatase | 0.032 | −2.14 | −2.79 | −3.21 |
| (AP) | | | | |
| CD63 | 0.032 | −1.93 | −2.43 | −3.26 |
| MMP-16/MT3-MMP | 0.032 | 1.04 | −1.20 | −1.55 |
| Cyclin C | 0.034 | −1.02 | −1.49 | −1.71 |
| Neuron Specific Enolase | 0.040 | −1.78 | −2.06 | −3.18 |
| SIRP a1 | 0.041 | −1.09 | −1.53 | −1.91 |
| Laminin B1/b1 | 0.042 | −1.59 | −1.99 | −3.23 |
| Amyloid Beta (APP) | 0.043 | −1.20 | −1.65 | −2.41 |
| SODD (Silencer of Death | 0.043 | −1.05 | −1.34 | −1.70 |
| Domain) | | | | |
| CDC37 | 0.047 | −1.37 | −1.67 | −2.28 |
| Gab-1 | 0.047 | −1.05 | −1.16 | −1.33 |
| E2F-2 | 0.047 | −1.19 | −1.97 | −3.36 |
| CD6 | 0.047 | −1.37 | −2.10 | −2.55 |
| Mast Cell Chymase | 0.047 | −1.28 | −2.22 | −3.04 |
| Gamma Glutamylcysteine | 0.047 | −1.17 | −1.70 | −2.32 |
| Synthetase(GCS) | | | | |

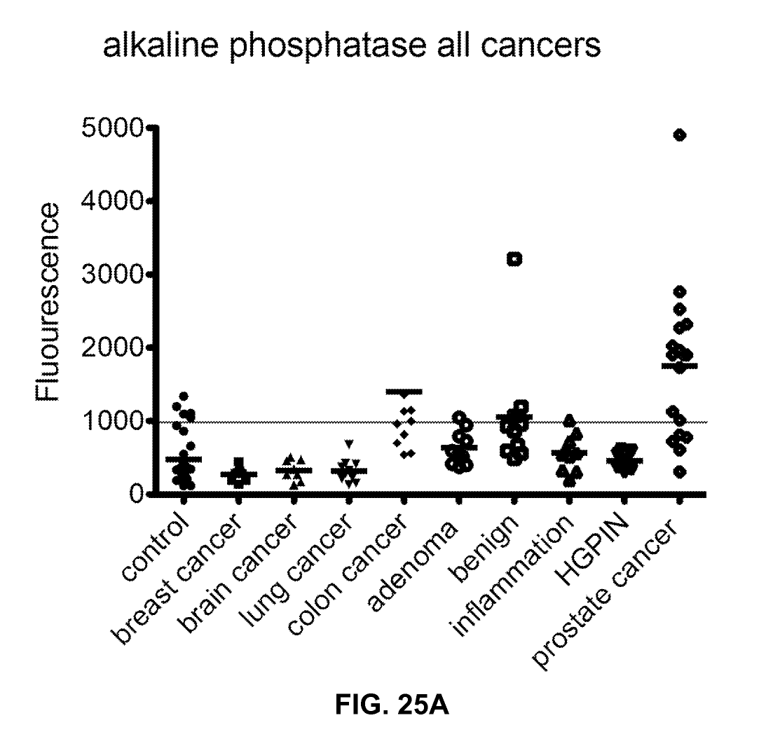

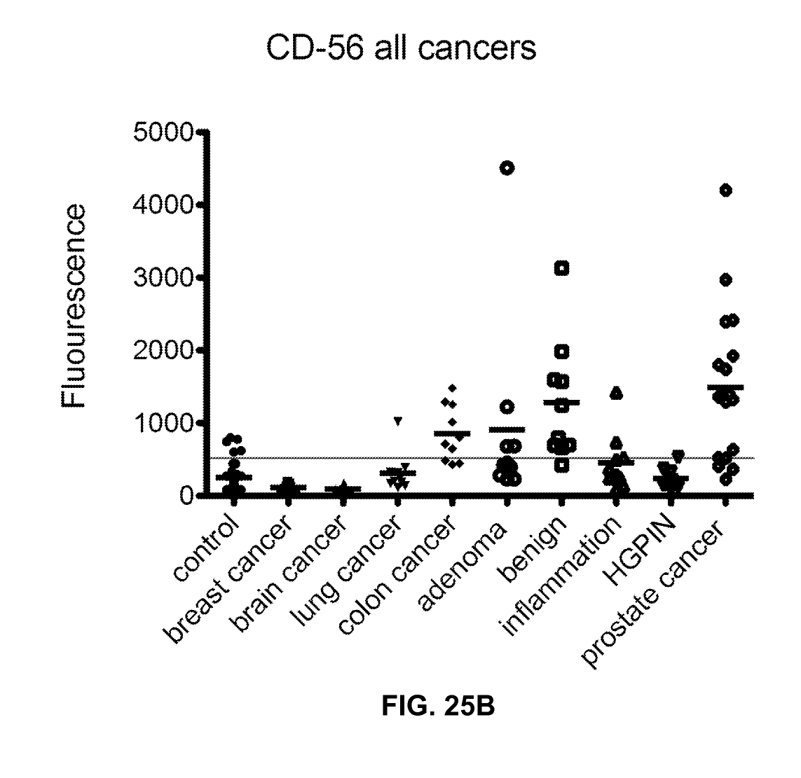

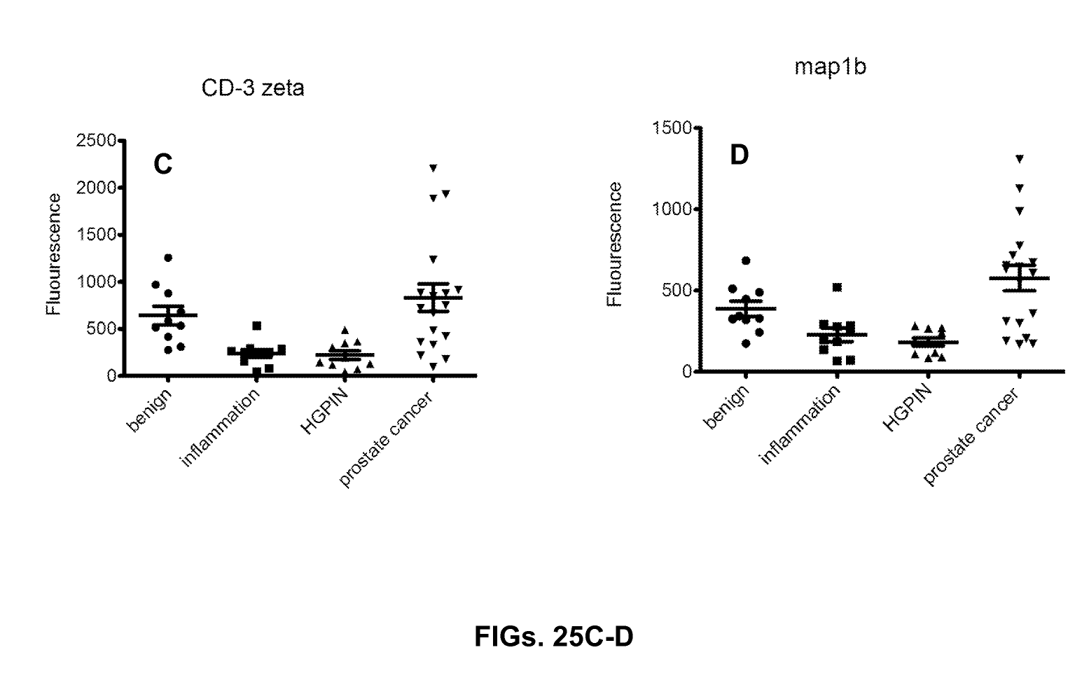

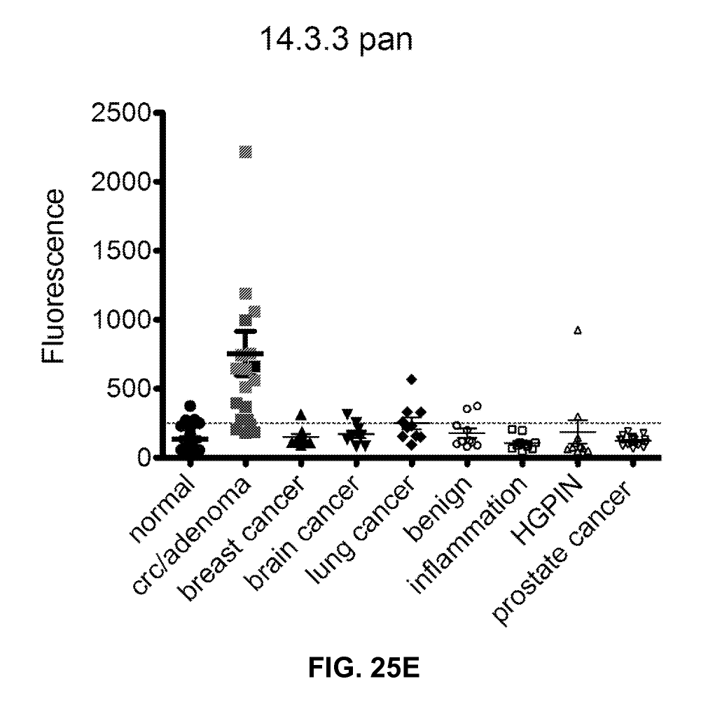

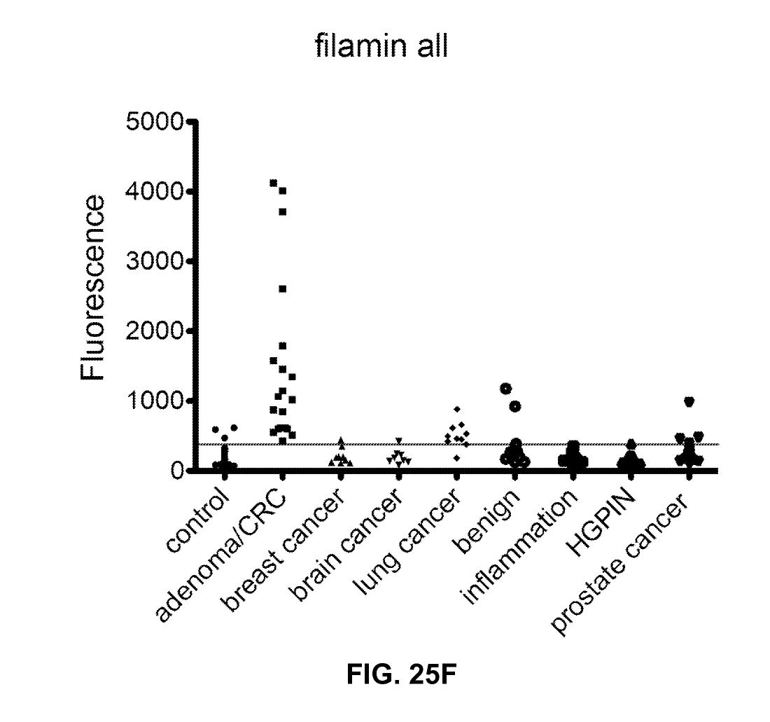

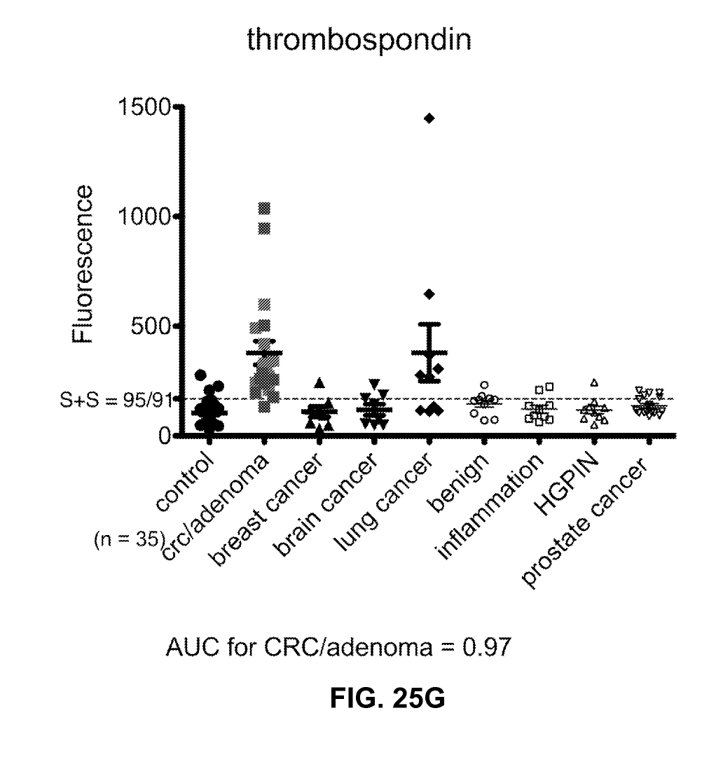

FIGS. 25A-G show levels of alkaline phosphatase (intestinal) (FIG. 25A), CD-56 (FIG. 25B), CD-3 zeta (FIG. 25C), map1b (FIG. 25D), 14.3.3 pan (FIG. 25E), filamin (FIG. 25F), and thrombospondin (FIG. 25G) associated with microvesicles from plasma of normal (non-cancer) control individuals, breast cancer patients, brain cancer patients, lung cancer patients, colorectal cancer patients, colon adenoma patients, BPH patients (benign), inflamed prostate patients (inflammation), HGPIN patients, and prostate cancer patients, as indicated in the figures. All samples were analyzed using antibody arrays as described in this Example.

As shown in FIGS. 25A-B, alkaline phosphatase (intestinal, ALPI) and CD56 biomarkers differentiate PCa from all other samples. The patients in this study include early stage cancers. CD-56 (CD56, NCAM) is related to EpCam. In addition, CD-3 zeta (FIG. 25C) and map1b (FIG. 25D) are effective biomarkers for distinguishing various prostate associated conditions, e.g., inflammation and HGPIN. In another embodiment, biomarkers for colorectal associated conditions include markers 14.3.3 pan (FIG. 25E), filamin (FIG. 25F), and thrombospondin (FIG. 25G), e.g., to differentiate colorectal cancer and adenoma from other cancers.

### Example 40

### Vesicle Sample Processing

This Example presents methods that can be used to analyze vesicles, e.g., cMVs, cell line exosomes, etc., using particle-based, flow cytometry, and other methods. The Example presents processing of plasma samples using depletion of highly abundant proteins prior to downstream analysis.

1.2 μm Plasma Filtration

1. Thaw 1 mL aliquots of plasma from −80C, pool them, and add 10% DMSO

2. Filter plasma through 1.2 μm filter plate

a. Stack 96-well plate on top of 96 well white, round bottom plate (Costar #3789)

b. Pre-wet number of wells needed with 100 μL 0.1 μm filtered PBS

c. Spin at 4,000 RPM in Eppendorf 5430R for 1 min

d. Remove PBS from wells in white plate

e. Add 50 μL plasma per well

f. Spin at 4,000 RPM in Eppendorf 5430R for 2 min

3. Remove plasma from wells into 1.5 mL microcentrifuge tubes

4. Store samples on ice

HSA/IgG Depletion Protocol

This protocol presents a method of human serum albumin (HSA) from a blood sample. The protocol uses the commercially available Pierce Albumin/IgG Removal Kit (#89875). Similar kits from other manufacturers can be employed.

1. Add 170 μL of resuspended resin (vortex 30 sec) to ten spin columns per sample (Cibacron Blue/Protein A)

2. Centrifuge 10,000 g for 1 min to remove storage buffer

3. In a separate tube, add 65 μL binding buffer+10 μL neat plasma×the number of spin columns per sample (715 μl binding buffer+110 μl 1.2 um filtered plasma)

(E8 Prep Requires Pre-Filtering Step)

4. Add 75 μL diluted sample to the resin of each of the 10 columns per sample

5. Vortex lightly to mix

6. Incubate on rotator for 10 min at room temp

7. Centrifuge 10,000 g for 1 min to collect flowthrough

8. Add flowthrough back to resin

9. Vortex lightly to mix

10. Incubate on rotator for 10 min at room temp

11. Centrifuge 10,000 g for 1 min to collect flowthrough

12. To wash, add 75 μl of binding buffer

13. Centrifuge 10,000 g for 1 min to collect wash in the same collection tube as flowthrough to combine (total volume=150 μl)

14. Pool the flowthrough/wash from all 10 of the columns per sample in a separate 1.5 mL microcentrifuge tube (total volume=1500 μl)

15. Concentrate the sample prior to Fc Receptor binding and staining

HSA Depleted Plasma Concentration Protocol

This protocol uses an Amicon Ultra-2 Centrifugal Filter Unit with Ultracel-50 membrane (# UFC205024PL).

1. Insert the Amicon Ultra-2 device into the filtrate collection tube

2. Prewet by adding 2 mL of Apogee 0.1 μm filtered water and centrifuge 2000 g for 2 min

3. Add 1500 μl of HSA depleted plasma and centrifuge @ 2500 g for 15 mins

4. Separate the filter device from the flowthrough collection tube

5. Recover concentrated sample by inverting the filter device and centrifuging @ 1000 g for 1 min

6. Transfer recovered concentrated sample from the collection tube to a separate 1.5 mL microcentrifuge tube

7. Adjust final volume to 1000 with 0.1 μm PBS

8. Store sample on ice

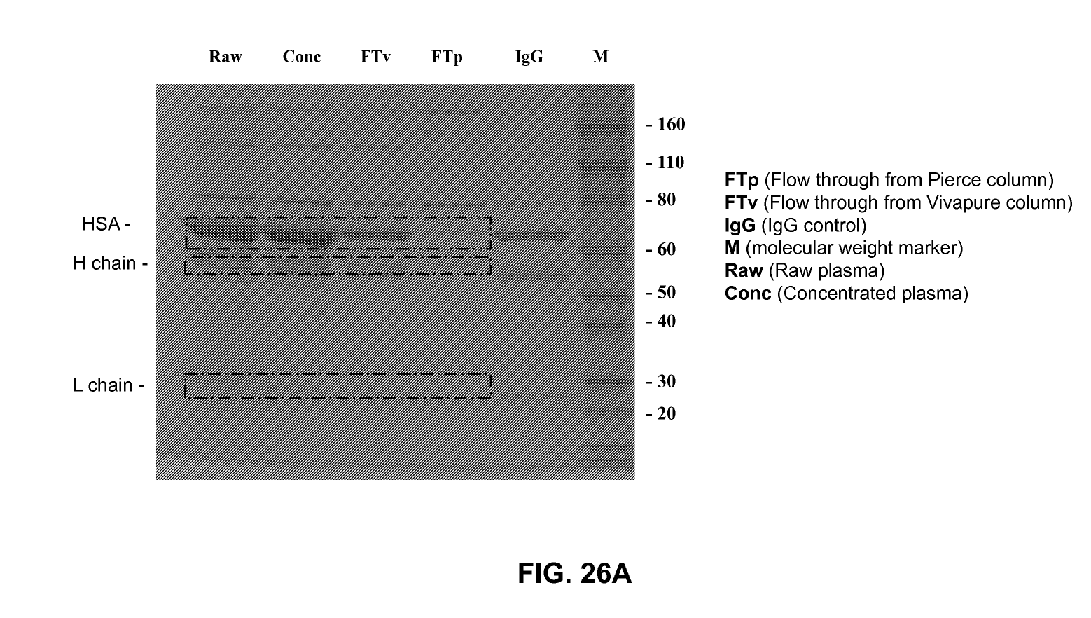

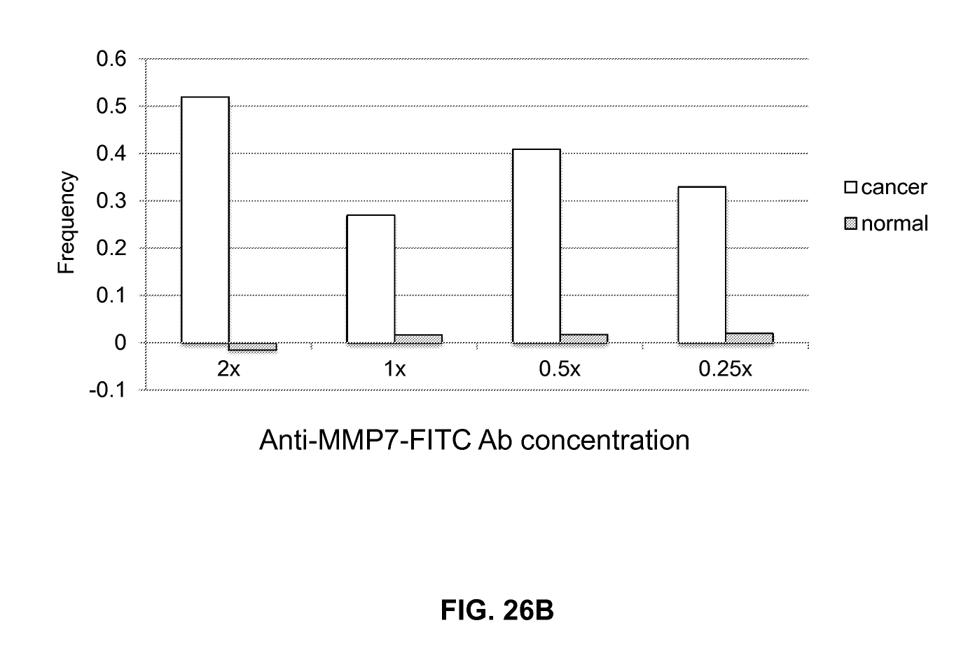

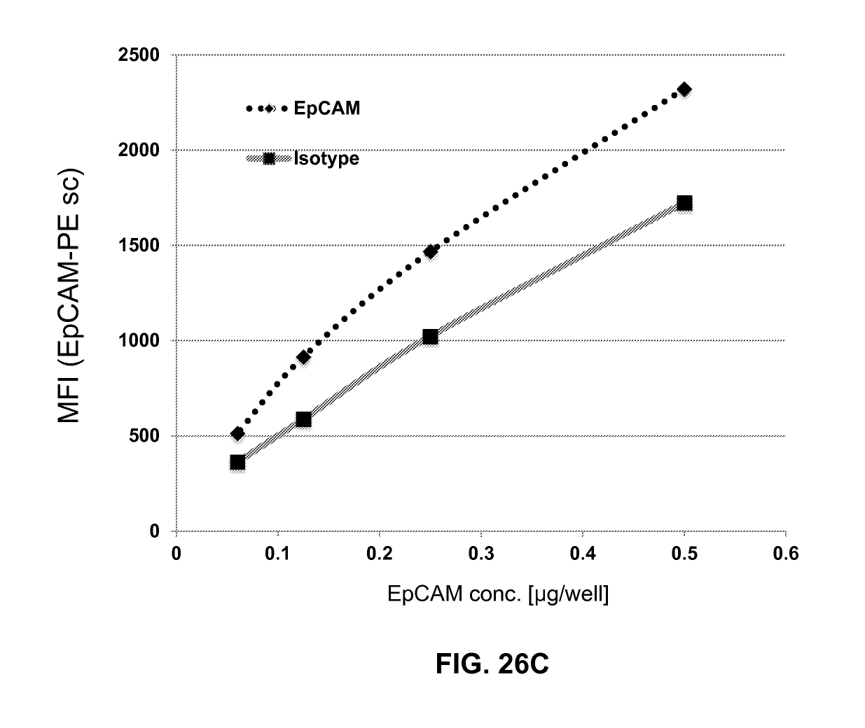

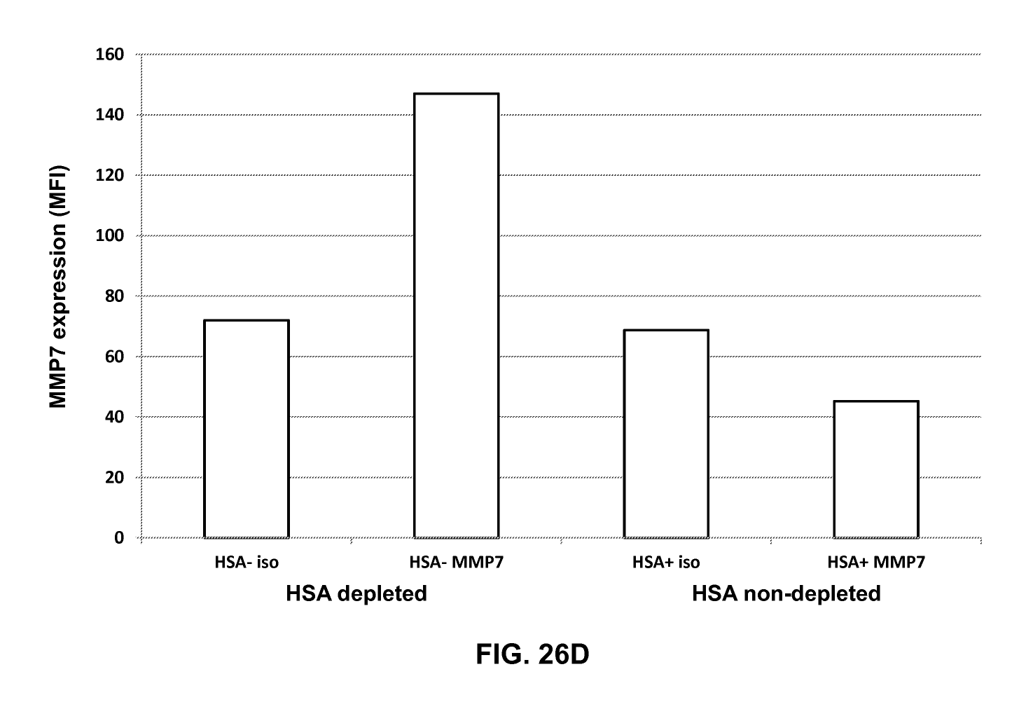

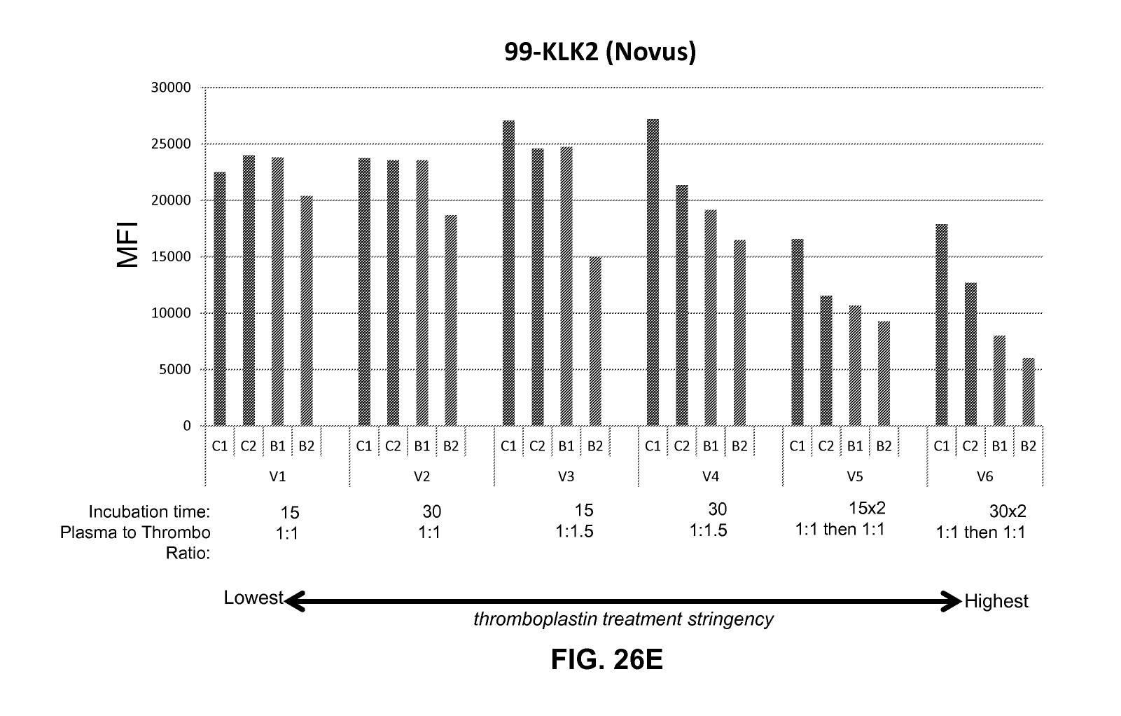

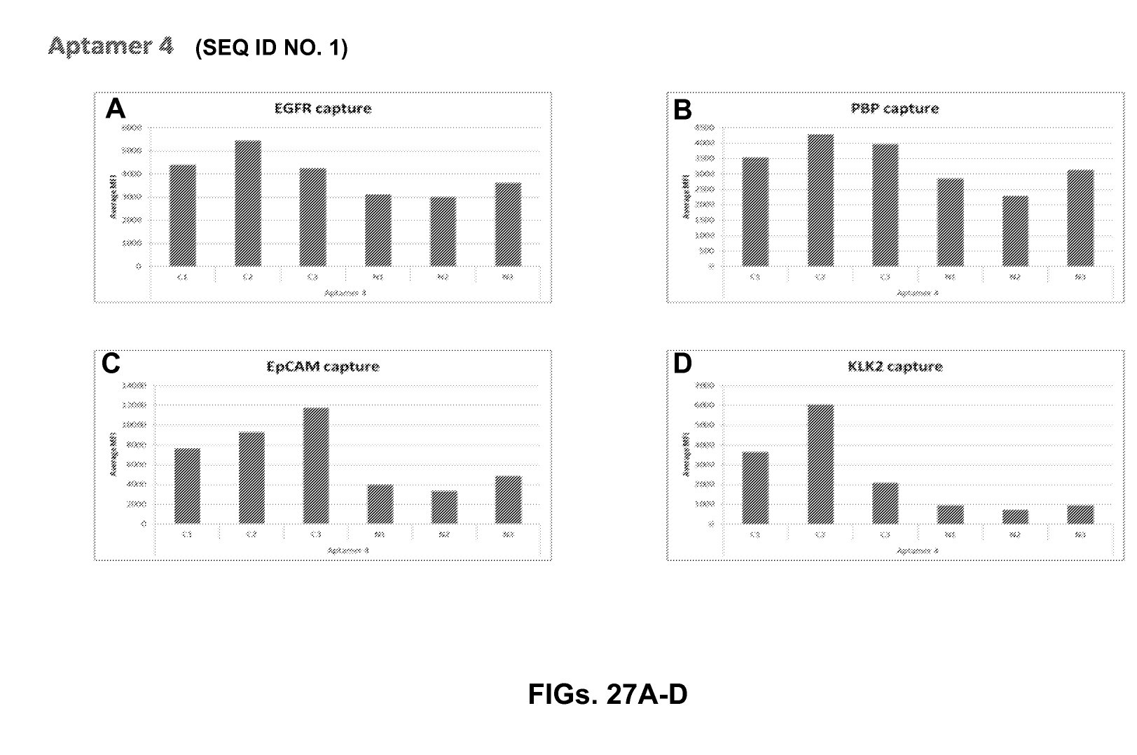

FIG. 26A illustrates a protein gel demonstrating removal of HSA and antibody heavy and light chains in the indicated samples. The columns in the gel are as follows: “Raw” (Plasma without any treatment); “Conc” (Plasma concentrated via nanomembrane filtration); “FTp” (Plasma flow through from treatment with Pierce Albumin and IgG Removal Kit, Thermo Fisher Scientific Inc., Rockford, Ill. USA); “FTv” (Plasma flow through from treatment with Vivapure® Anti-HSA/IgG Kit from Sartorius Stedim North America Inc., Edgewood, N.Y. USA); “IgG” (IgG control); “M” (molecular weight marker).

Fibrinogen Depletion

1. Bring Thromboplastin D (solid stock, Thermo Scientific) to room temperature. Dissolve in 4 ml of distilled water or use stock prepared not later than 1 week

2. Pipet desired volume of plasma and add an equal volume of Thromboplastin D. Mix well, incubate at 37° C. for 15 min

3. Centrifuge at 10,000 rpm at room temperature for 5 min

4. Transfer supernatant into a fresh tube. To recover maximum sample, disturb and squeeze pellet against the walls (it will become more compact once touched)

5. Measure the volume of the collected supernatant

The filtered and protein depleted sample can be used for further analysis. For example, vesicles in the sample can be isolated then assessed using various methods disclosed herein or known in the art. Vesicles can be isolated using a number of methods disclosed herein or known in the art, including without limitation ultracentrifugation (see, e.g., Examples 1-2), filtration (see, e.g., Examples 6, 17, 20), immunoprecipitation (see, e.g., Examples 30, 32), or use of a commercial kit such as the ExoQuick™ kits (System Biosciences, Mountain View, Calif. USA) or Total Exosome Isolation kits from Invitrogen/Life Technologies (Carlsbad, Calif. USA).

ExoQuick Exosome Isolation

1. Mix fibrinogen depleted (serum-like) sample with 0.25 volume of ExoQuick solution.

2. Centrifuge mixture at 1500 g for 30 min at room temperature or 4° C.

3. Vesicles appear in yellowish pellet. Remove supernatant.

4. Centrifuge for additional 5 min at 1500 g.

5. Discard supernatant, do not to disturb the pellet.

6. Add 50 μl of distilled water to the pellet, let sit for 5 min, dissolve precipitate by pipetting.

7. Once the pellet is resuspended, the vesicles are ready for downstream analysis or further purification through affinity methods.

8. Keep isolated vesicles at 2° C. to 8° C. for up to 1 week, or at <20° C. for long-term storage.

Total EXosome ISolation (TEXIS)

1. Mix fibrinogen depleted (serum-like) sample with 0.2 volume of TEXIS solution.

2. Mix the sample/reagent mixture well either by vortexing or pipetting up and down until there is a homogenous solution. Note: The solution should have a cloudy appearance.

3. Incubate the sample at 2° C. to 8° C. for 30 minutes.

4. After incubation, centrifuge the sample at 10,000×g for 10 minutes at room temperature.

5. Aspirate and discard the supernatant. Vesicles are contained in the pellet at the bottom of the tube.

6. Use a pipette tip to completely resuspend the pellet in a convenient volume of distilled water (50 to 100 μl).

7. Once the pellet is resuspended, the vesicles are ready for downstream analysis or further purification through affinity methods.

8. Keep isolated vesicles at 2° C. to 8° C. for up to 1 week, or at <20° C. for long-term storage.

Vesicles isolated by the methods above can be assessed using any number of assays disclosed herein or known in the art, including without limitation immunoassays (see, e.g., Example 28), particle-based assays (see, e.g., Examples 4, 5, 20, 22, 28), immunoprecipation (see, e.g., Examples 30, 32) and flow analysis (see, e.g., below; see also Examples 19, 31, 33).

Flow Cytometry: TruCount Protocol for Filtered Neat Plasma Samples

1. Remove one TruCount tube per sample from 4C storage and verify that there is a small white bead pellet at the bottom of the tube below the metal insert

2. Protect TruCount tubes from light using metal foil and allow them to equilibrate to RT (15 mins)

3. Combine 90 μl of 0.1 μm filtered PBS+10 μl of concentrated HSA depleted plasma in a 1.5 mL microcentrifuge tube

4. Mix by vortexing and add the 100 μl PBS+ sample mixture directly above the metal insert at the bottom of the TruCount tubes

5. Verify after >1 min that the white bead pellet has dissolved, if not, dissolve the pellet by pulse vortexing until the pellet is no longer visible

6. Once the pellet is completely dissolved, protect the TruCount tubes from light with metal foil and incubate for 15 mins @ RT

7. Following the first incubation, adjust the TruCount sample volume from 100 μl up to 300 μl total with 0.1 μm filtered PBS (200 μl) and pulse vortex to mix

8. Protect the TruCount tubes from light with metal foil and incubate for an additional 15 mins @ RT

9. Vortex briefly, immediately prior to analysis on the Apogee

10. Run samples @ 200 μl/min flow rate and 300 μl aspiration volume

Staining Plasma for Flow Analysis

1. Aliquot 0.25x10e6 events per well

2. Add 15 μl of Fc receptor blocking ebiosciences (cat #16-9161-73) store sample overnight 4° C.

3. Add Antibody cocktail per well and incubate for 30 min in dark on ice.

4. Bring up to 300 μl with filtered PBS.

5. Run 300 μl of stained sample on Apogee @ 2000/min flow rate and 300 μl aspiration volume.

6. Flow Jo analysis.

FIG. 26B shows an example of using the HSA/IgG depletion and flow cytometry protocols to detect cMVs from the peripheral blood of prostate cancer and normal patients. The cMVs were detected using Anti-MMP7-FITC antibody conjugate (Millipore anti-MMP7 monoclonal antibody 7B2) and the flow cytometry protocol above. The plot shows the frequency of events detected versus concentration of the detection antibody.

As noted, the methods for sample treatment to remove highly abundant proteins can also be applied to particle-based assays. FIG. 26C shows EpCam expression in human serum albumin (HSA) depleted plasma sample. The x-axis refers to concentration of EpCam+ vesicles in various aliquots. The Y axis illustrates median fluorescent intensity (MFI) detected in a microbead assay using PE labeled anti-EpCAM antibodies to detect the vesicles. “Isotype” refers to detection using PE anti-IgG antibodies as a control. FIG. 26D is similar to FIG. 26C except that PE-labeled anti-MMP7 antibodies were used to detect the microvesicles. Shown are samples that were pre-treated to remove HSA (“HSA depleted”) or not (“HSA non-depleted”). “iso” refers to the anti-IgG antibody controls. As observed in the figure, HSA depletion had no effect on the background MFI observed using the IgG control. However, there was a ˜3.5-fold increase in MFI of MMP7+ vesicles after HSA depleteion. FIG. 26E illustrates detection of vesicles in plasma after treatment with thromboplastin to precipitate fibrin. The Y axis illustrates median fluorescent intensity (MFI) detected in a microbead assay using bead-conjugated anti-KLK2 to capture the vesicles and a PE labeled anti-EpCAM aptamer to detect the vesicles. The X-axis groups 4 plasma samples (cancer sample C1, cancer sample C2, benign sample B1, benign sample B2) into 6 experimental conditions, V1-V6. As indicated by the thromboplastin incubation time and concentration below the plot, the thromboplastin treatment stringency increased from V1-V6. As observed in the figures, the ability to distinguish cancer samples C1-C2 from benign sample B1-B2 improved with the stringency of the thromboplastin treatment.

### Example 41

### Microbead Assay for Detection of Circulating Microvesicles (cMV)

A subset of marker pairs in Example 37 (see Table 28) were used to further assess EpCAM as a detector agent. Methodology was as described in the Examples above. Binding agents to ADAM-10, BCNP, CD9, EGFR, EpCam, IL1B, KLK2, MMP7, p53, PBP, PCSA, SERPINB3, SPDEF, SSX2, and SSX4 were used for capture of the microvesicles and binding agents to PCSA and EpCAM were used as detectors. Briefly, capture agents were conjugated to microbeads and incubated with patient plasma samples. Fluorescently labeled detector agents were used to detect the antibody-captured microvesicles. Binding agents used are those described above except that both EpCAM antibody and aptamer detector agents were used. The samples comprised 5 plasma samples from men with positive biopsy for prostate cancer (PCa) and 5 men with negative biopsy for prostate cancer (i.e., the controls). MFI values were compared between the PCa and control samples to assess the ability of the capture-binding pairs to detect and distinguish microvesicles in the prostate cancer cancers and controls. The performance of individual marker pairs and marker panels was assessed.

PE-labeled binding agents to three detector agents were used, comprising: 1) anti-EpCAM antibody; 2) anti-PCSA antibody; 3) anti-EpCAM aptamer. Combinations of detector agents along with microbead-tethered capture agents are shown in Table 44. In the table, the capture and/or detector agents comprised antibodies that recognize the indicated targets unless noted as aptamers. The first row identifies the Detector agents. Beneath each detector is the list of capture agents used with the detector.

TABLE 44
Capture and Detector Agent Combinations

| EpCAM | EpCAM aptamer | PCSA |
|---|---|---|
| EpCAM | EpCAM | EpCAM |
| KLK2 | KLK2 | KLK2 |
| PBP | PBP | PBP |
| SPDEF | SPDEF | SPDEF |
| SSX2 | SSX2 | SSX2 |
| SSX4 | SSX4 | SSX4 |
| ADAM-10 | ADAM-10 | ADAM-10 |
| SERPINB3 | SERPINB3 | SERPINB3 |
| PCSA | PCSA | PCSA |
| p53 | p53 | p53 |
| MMP7 | MMP7 | MMP7 |
| IL1B | IL1B | IL1B |
| EGFR | EGFR | EGFR |
| CD9 | CD9 | CD9 |
| BCNP | BCNP | BCNP |

ROC curves were constructed for each capture-detector pair. The performance of individual capture agents to EpCAM, KLK2, PBP, SPDEF, SSX2 and SSX4 along with EpCAM antibody detector are shown in Table 45. In the table, AUC is the area under the curve of the ROC curve.

TABLE 45
Capture Agent - EpCAM Detector Performance

| Capture Target | Vendor | Cat. No. | AUC |
|---|---|---|---|
| EpCAM | R&D Systems | MAB9601 | 0.72 |
| KLK2 | Novus Biologicals | H00003817-M03 | 1.00 |
| PBP | Novus Biologicals | H00005037-M01 | 0.64 |
| SPDEF | Novus Biologicals | H00025803-M01 | 0.80 |
| SSX2 | Novus Biologicals | H00006757-M01 | 0.92 |
| SSX4 | Novus Biologicals | H00006759-M02 | 1.00 |

As observed in Table 45, all individual marker pairs demonstrated ability to distinguish PCa and control samples. SERPINB3 capture also had an AUC value of 1.0 (i.e., perfect ability to distinguish cancer and normals) and EGFR capture had an AUC of 0.64.

Table 46 shows the results of several dual pair panels of markers. A multivariate model was used to assess the ability of the panels to distinguish distinguish PCa and control samples using the ROC AUC as a performance metric. In Table 46, the panels comprised Capture Target 1-EpCAM detector, and Capture Target 2-EpCAM detector. There is no significance to the designation of Target 1 or 2 (e.g., Capture Target 1=SSX4 and Capture Target 2=EpCAM is equivalent to Capture Target 2=SSX4 and Capture Target 1=EpCAM). The AUC for the panels should be at least as high as the worst performing individual marker in the panel. Indeed, the panels provided improved performance (i.e., higher AUC value) over the individual markers. Even in cases where some markers showed perfect discrimination as individual capture targets (i.e., AUC=1.0; e.g., SSX4, KLK2, SERPINB3), the panels may still provide real world benefit through reduced assay variance or other factors.

TABLE 46
Dual Capture Agent - EpCAM Detector Performance

| Capture Target 1 | Capture Target 2 | AUC |
|---|---|---|
| SSX4 | EpCAM | 1.00 |
| SSX4 | KLK2 | 1.00 |
| SSX4 | PBP | 1.00 |
| SSX4 | SPDEF | 1.00 |
| SSX4 | SSX2 | 1.00 |
| SSX4 | EGFR | 1.00 |
| SSX4 | MMP7 | 1.00 |
| SSX4 | BCNP1 | 1.00 |
| SSX4 | SERPINB3 | 1.00 |
| SSX4 | Any other marker | 1.00 |
| KLK2 | EpCAM | 1.00 |
| KLK2 | PBP | 1.00 |
| KLK2 | SPDEF | 1.00 |
| KLK2 | SSX2 | 1.00 |
| KLK2 | EGFR | 1.00 |
| KLK2 | MMP7 | 1.00 |
| KLK2 | BCNP1 | 1.00 |
| KLK2 | SERPINB3 | 1.00 |
| KLK2 | Any other marker | 1.00 |
| PBP | EGFR | 0.81 |
| PBP | EpCAM | 0.78 |
| PBP | SPDEF | 0.90 |
| PBP | SSX2 | 0.96 |
| PBP | SERPINB3 | 1.00 |
| PBP | MMP7 | 0.80 |
| PBP | BCNP1 | 0.78 |
| EpCAM | SPDEF | 0.87 |
| EpCAM | SSX2 | 0.95 |
| EpCAM | SERPINB3 | 1.00 |
| EpCAM | EGFR | 0.75 |
| EpCAM | MMP7 | 0.75 |
| EpCAM | BCNP1 | 0.72 |
| SPDEF | SSX2 | 0.98 |
| SPDEF | SERPINB3 | 1.00 |
| SPDEF | EGFR | 0.87 |
| SPDEF | MMP7 | 0.89 |
| SPDEF | BCNP1 | 0.87 |
| SSX2 | EGFR | 0.95 |
| SSX2 | MMP7 | 0.96 |
| SSX2 | BCNP1 | 0.95 |
| SSX2 | SERPINB3 | 1.00 |
| SERPINB3 | EGFR | 1.00 |
| SERPINB3 | MMP7 | 1.00 |
| SERPINB3 | BCNP1 | 1.00 |
| SERPINB3 | Any other marker | 1.00 |
| EGFR | MMP7 | 0.81 |
| EGFR | BCNP1 | 0.75 |
| MMP7 | BCNP1 | 0.78 |

The data in Tables 45 and 46 was obtained using a PE-labeled anti-EpCAM antibody as detector. FIGS. 27A-D illustrates the use of an anti-EpCAM aptamer (i.e., Aptamer 4; 5′-CCC CCC GAA TCA CAT GAC TTG GGC GGG GGT CG (SEQ ID NO. 1)) to detect the microvesicle population captured with antibodies to the indicated microvesicle antigens (FIG. 27A: EGFR; FIG. 27B: PBP; FIG. 27C: EpCAM; FIG. 27D: KLK2). The aptamer was biotin-conjugated then labeled by binding with streptavidin-phycoerytherin (SAPE). The figure shows average median fluorescence values (MFI values) for three illustrative prostate cancer (C1-C3) and three normal samples (N1-N3) in each plot. Similar ability to separate cancers and normals was observed using either antibody or aptamer detector agents.

As seen in Table 45, assays using individual capture targets showed excellent ability to distinguish cancers and normals. Table 46 further demonstrates that panels assessing at least two capture targets can further improve assay performance.

### Example 42

### Identification and Implications of Transcription Factors in Circulating Microvesicles from Cancer Patients

Circulating microvesicles (cMV) are small membrane bound particles that play important roles in the pathogenesis of many human diseases including heart disease, autoimmunity, and cancer. cMV are known to contain proteins and RNA molecules derived from their cell of origin. The transcription factors ATF3 and WT-1 have been detected in urine microvesicles from patients with acute kidney injury. Other transcription factors (TF) identified within cancer-associated cMV including c-Myc, p53, AEBP1, and HNF4a.

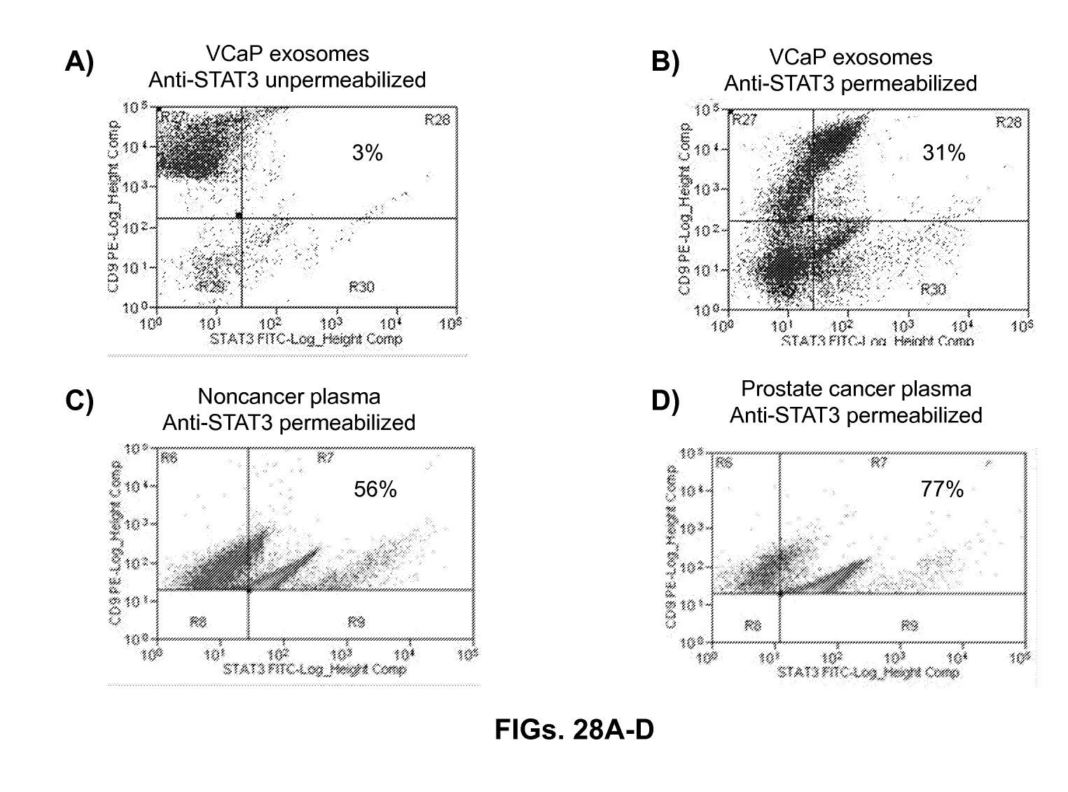

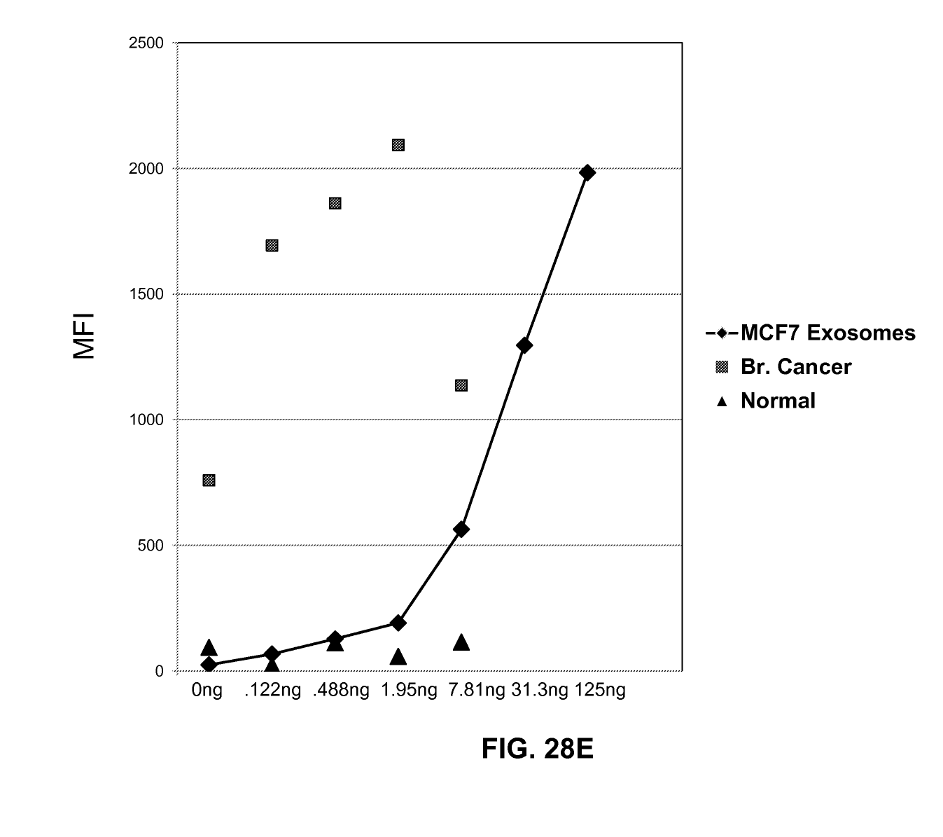

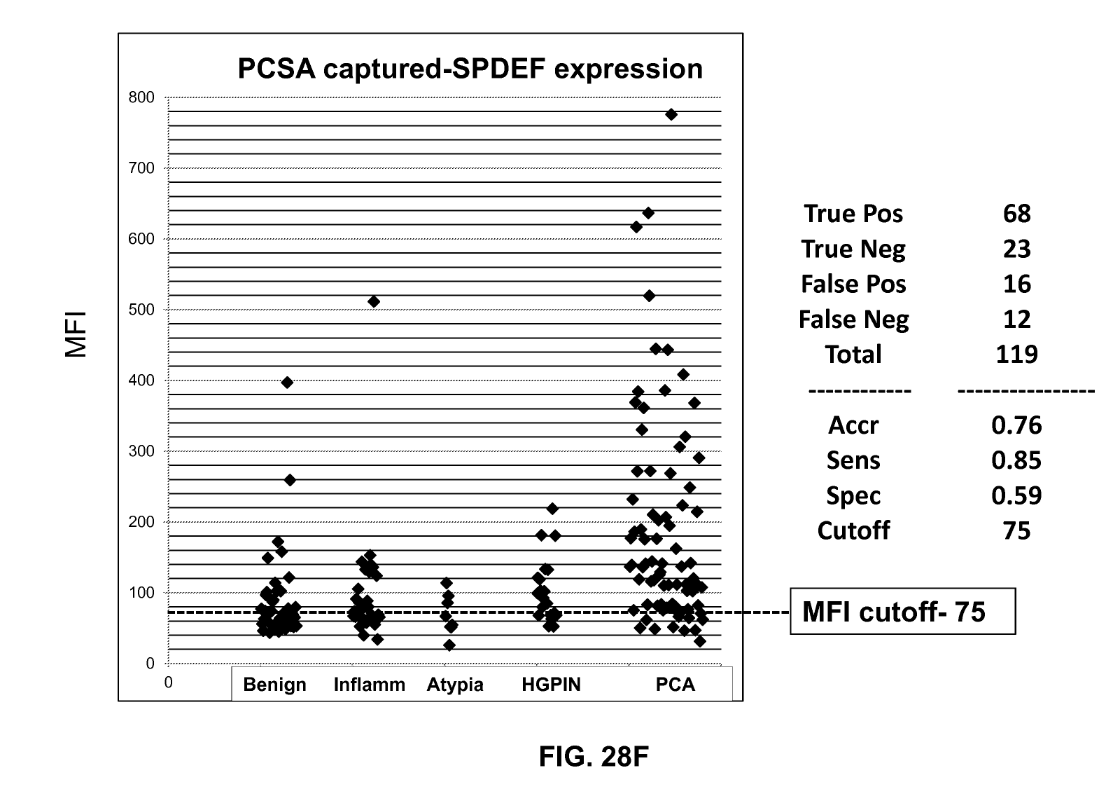

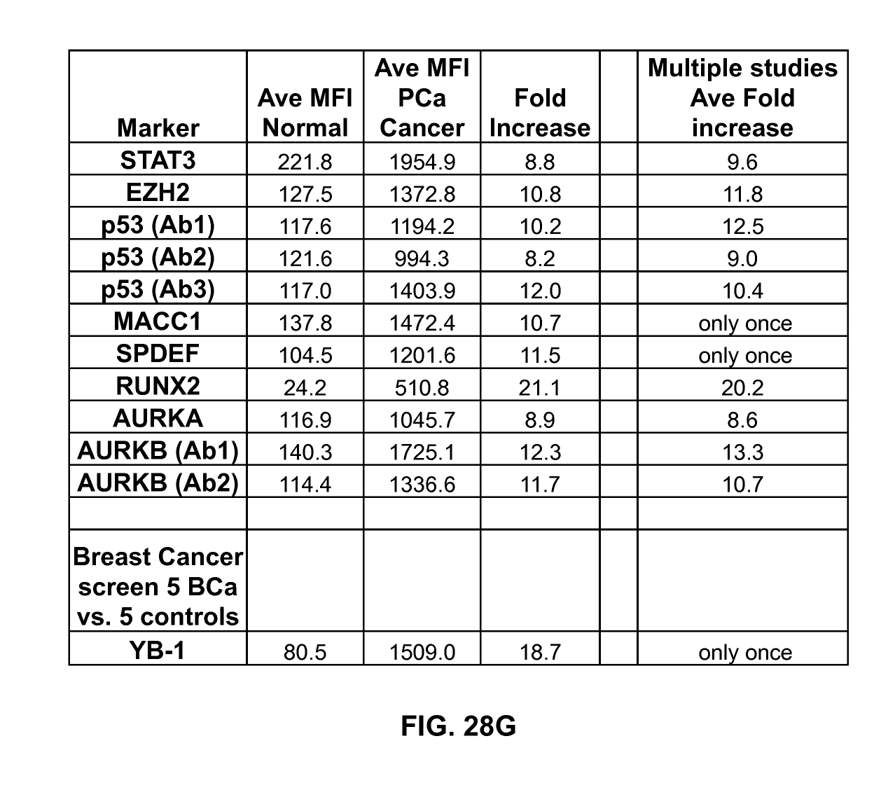

Using multi-parametric flow cytometry and an antibody sandwich assay, several TF and Aurora kinases were identified in prostate cancer (PCA) cMV. STAT3 was identified in permeabilized cMVs from the PCA cell line VCaP, and STAT3+cMVs from PCA patient plasma samples was elevated when compared to plasma samples from non-cancer males. See FIGS. 28A-D. The data in FIGS. 28A-B show that STAT3 is found in VCaP-derived cMVs after permeabilization, implying internal localization of this TF. Additionally, analysis on isolated cMVs from plasma of breast cancer patients and non-cancer female plasma revealed that the signal for a Y-box cell cycle-associated TF, Y box binding protein 1 (YB-1), was higher in breast cancer cMV compared to those from non-cancer female controls. The data in FIG. 28E shows a standard curve for breast cancer cell-derived cMVs for YB-1 and that breast cancer plasma has higher levels of YB-1+cMVs compared with healthy female controls. A prostate tissue-specific ETS-associate transcription factor, SAM pointed domain-containing Ets transcription factor (SPDEF), was elevated in cMVs from biopsy-confirmed PCA plasma compared to plasma from men with non-cancer prostate conditions (obtained from men undergoing prostate biopsies to rule out PCA). FIG. 28F summarizes SPDEF expression on prostate tissue-derived cMVs from men with a range of prostate disease diagnoses. Fluorescently labeled anti-SPDEF antibodies were used to detect cMV-associated SPDEF in the plasma samples. The mean fluorescence of SPDEF in men with benign diagnosis (n=39) was 91, inflammatory prostatic disease (n=29) was 101, cellular atypia (n=8) was 68, and HGPIN (n=21) was 102. In contrast, the mean fluorescence of cMV-associated SPDEF in samples from PCA patients (n=80) was 188. This data reveals a trend for increasing higher SPDEF expression in cMVs with increasing risk of prostate malignancy. Thus, SPDEF in cMV may serve as a target for PCA therapeutics. Lower cellular SPDEF has been associated with more aggressive phenotypes and higher Gleason score. Without being bound by theory, these observations suggest that shedding of SPDEF into cMV may play a role in PCA progression by actively reducing cellular levels of this TF. FIG. 28G shows a table that summarizes TF expression on cMVs from prostate or breast cancer plasma and the ratio compared with non-cancer controls.

Like miRNAs and lncRNAs, transcription factors can influence the expression of multiple proteins and can have a major impact on cell biology. TFs can directly alter the transcription rate of specific genes and have also been shown to interact with other proteins that have significant biologic impacts. These include cancer associated properties such as epigenetics, cell cycle regulation, DNA repair, anti-apoptosis, differentiation, proliferation, angiogenesis and even steroid hormone response. All of the TFs evaluated in this Example (i.e., STAT3, EZH2, p53 (Ab1), p53 (Ab2), p53 (Ab3), MACC1, SPDEF, RUNX2, YB-1) and kinases (AURKA, AURKB (Ab1), AURKB (Ab2)) had MFI levels higher in cancer-associated cMVs than in controls. See FIG. 28G. Without being bound by theory, higher level of TFs in cancer-associated cMVs may contribute to the “field effect” seen in normal tissue surrounding tumors, promote invasion/metastases and contribute to cancer progression in patients.

### Example 43

### The Influence of Bowel Preparation and Colonoscopy on the Secretion of Circulating Microvesicles

Circulating microvesicles (cMV) are small membrane structures that are secreted by multiple cell types and have been found in blood, urine, saliva and other body fluids. cMV transfer information from cell to cell by transporting selected proteins, mRNA and microRNA that correlate to their cell of origin.

The number of cMV shed by cells increases when the cells are biochemically stressed. To determine if the physical stress associated with bowel preparation and colonoscopy would result in an increase in the amount of colon cMV shed into the vascular system, blood was collected prospectively from 27 individuals at different time points and processed into plasma. Five time points were chosen for this study to establish the basal level of colon cMV, the effect of the procedure on cMV levels, and when cMV levels return to baseline. Specifically, the five time points were: 1) before bowel preparation; 2) after bowel preparation and before colonoscopy; 3) one day post colonoscopy; 4) 3-5 days post colonoscopy; and 5) one week post colonoscopy. The cMV levels were profiled using 115 protein markers that have been correlated to colon tissue, or colon cancer in the literature.

Blood was collected in K2-EDTA tubes and centrifuged at room temperature to isolate the plasma layer. Plasma samples were then immediately frozen and stored at or below −20° C. until tested. For each sample the cMVs were enriched by ultrafiltration and microbead immunoassay was used to detect cMVs. This assay is based on the antibody capture of cMVs and subsequent detection of the captured cMV by phycoerythrin labeled anti-tetraspanin antibodies. Capture antibodies included antibodies to tetraspanins CD9, CD63 and CD81, and to CD10, a membrane-bound metalloproteinase.

There was no statistical difference between any of the time points, suggesting that neither bowel preparation nor colonoscopy influence the secretion and composition of cMV; thus, the physical stress generated by the colonoscopy procedure does not appear to influence the secretion of colon cMV.

### Example 44

### Multi-Color Flow Cytometric Analysis of Cancer-Derived Microvesicles Reveals a Unique Subpopulation Ratio in Plasma from Prostate Cancer Patients

Circulating microvesicles (cMV) are cell-derived vesicles that can be isolated from many biofluids and culture media. Previous studies have shown that cMV are released by several cell types including immunocytes, endothelial, embryonic, tumor cells and also platelets. cMV in blood are a source of potential biomarkers of disease diagnosis and progression. The purpose of this study was to determine whether exposed biomarkers on the surface of cMV from processed plasma could distinguish prostate cancer microvesicles from atypia, high grade prostatic intraepithelial neoplasia (HGPIN), benign or prostate inflammation.

Isolated cMV from positive biopsy cancer patient blood were stained with a panel of specific conjugated antibodies to compare phenotype, frequency and marker expression. Plasma samples were collected prospectively prior to biopsy. The distribution of the cohort included 80 men with previously undiagnosed prostate cancer (current biopsy), 13 men with previously diagnosed prostate cancer and under active surveillance (previous biopsy), 6 atypia, 23 HGPIN, 28 inflammation, 49 benign, and 25 normal (no known prostate disorder) plasma samples. The cMV from these patients were analyzed by multi-color flow cytometry. Subpopulations of cMV were determined based on multiple combination of markers expression through proper gating.

Microvesicles from the plasma samples were obtained from patients and healthy donors by a blood draw and ultrafiltration as described in the Examples above. The microvesicles were collected and processed for staining with a cocktail of fluorochrome-conjugated antibodies. Microvesicle surfaces were stained with 1 μg of fluorochrome conjugated monoclonal antibodies cocktail: APC-EpCAM, PE-PCSA, PE-Cy7-Muc2 and PE-Cy7-Adam10 for 30 min on ice before acquisition. BD FACSCanto™ II Flow cytometer was used to acquired all data in this study. Data analysis was performed with Flow Jo v9.4 software (Tree Star, Inc.)

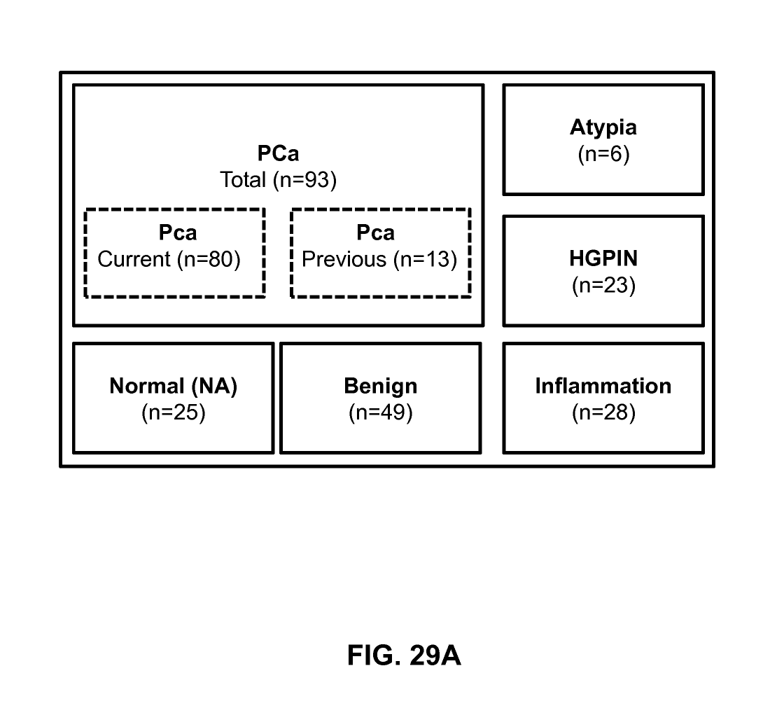

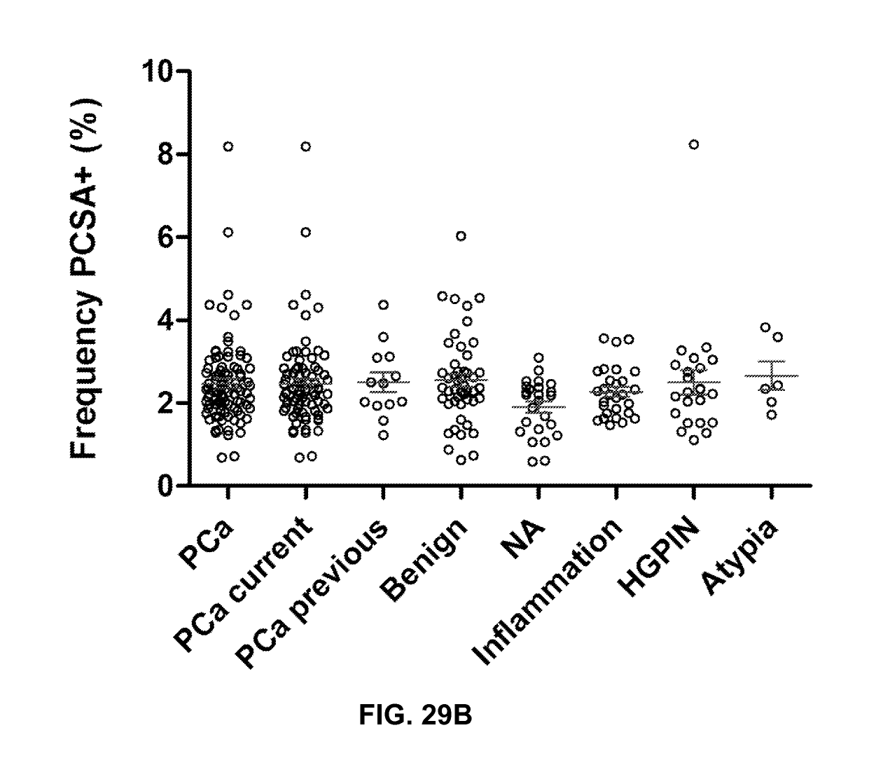

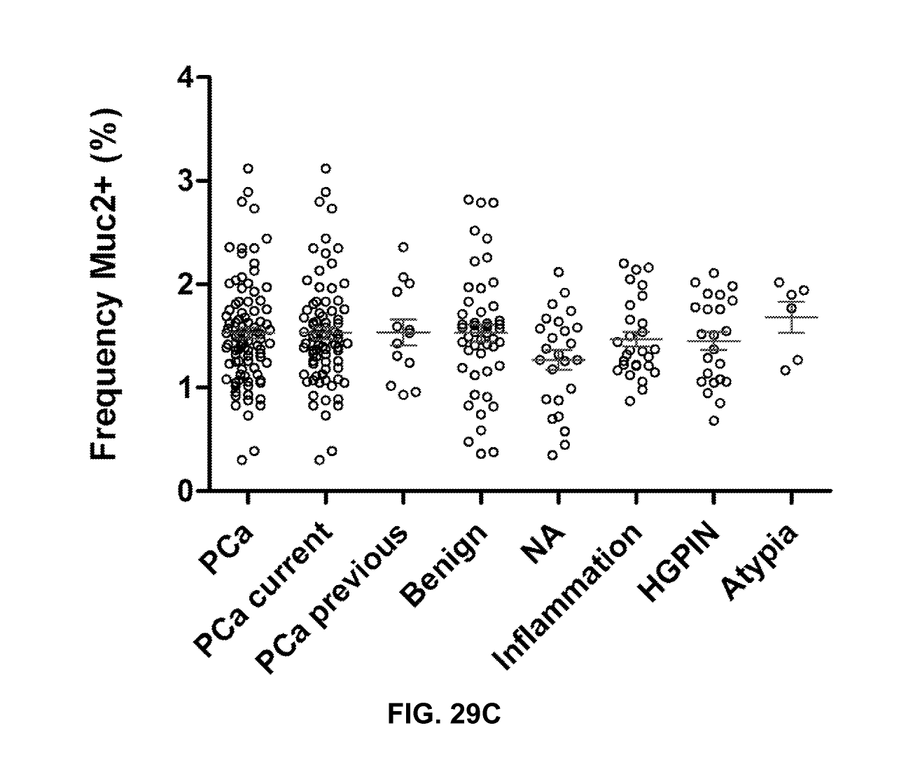

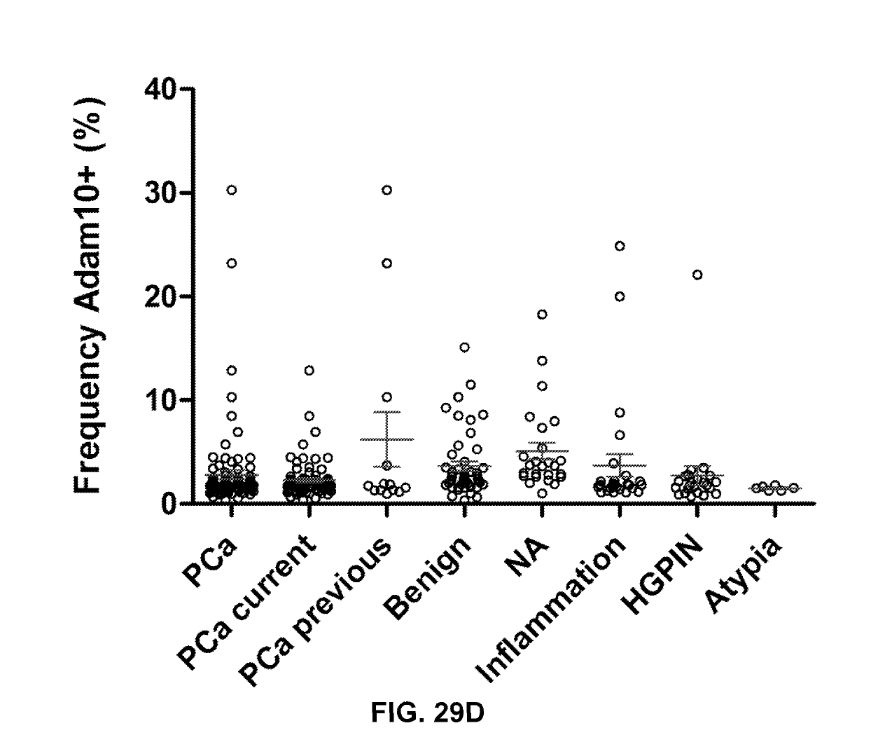

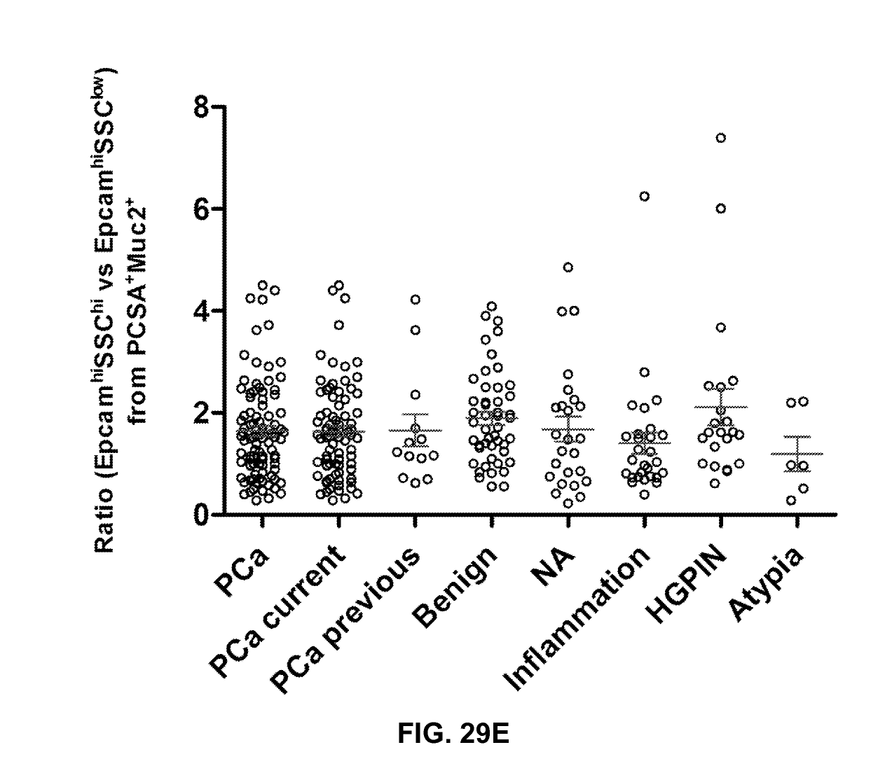

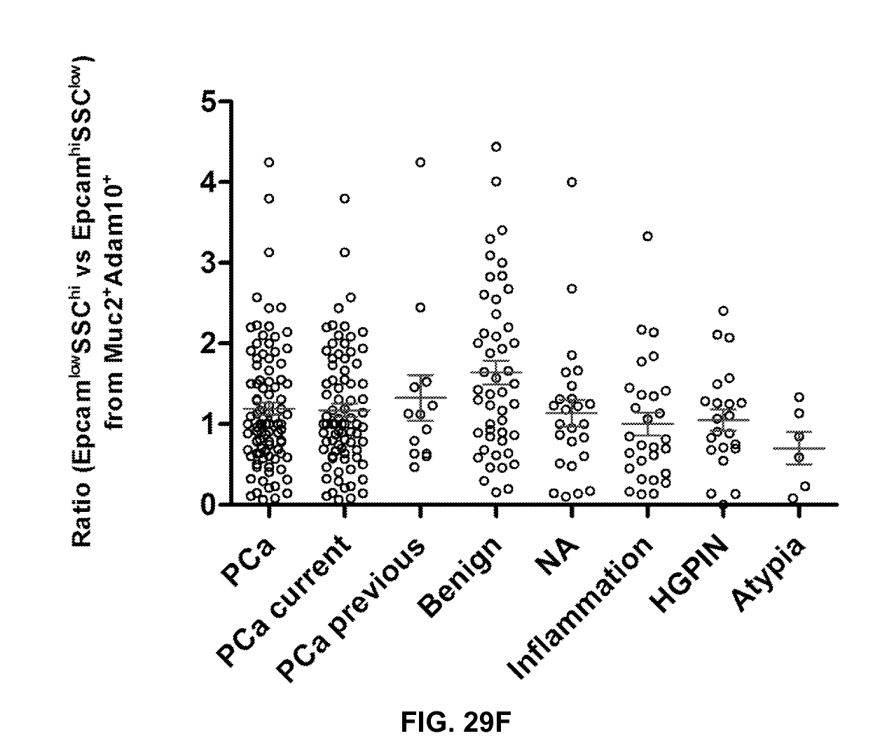

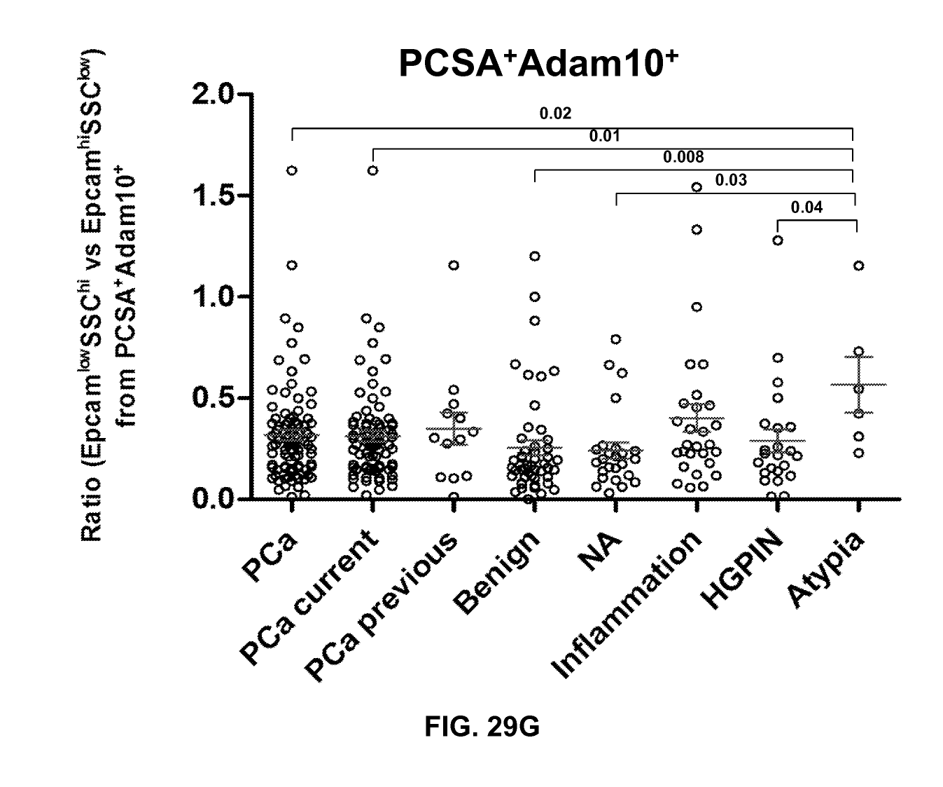

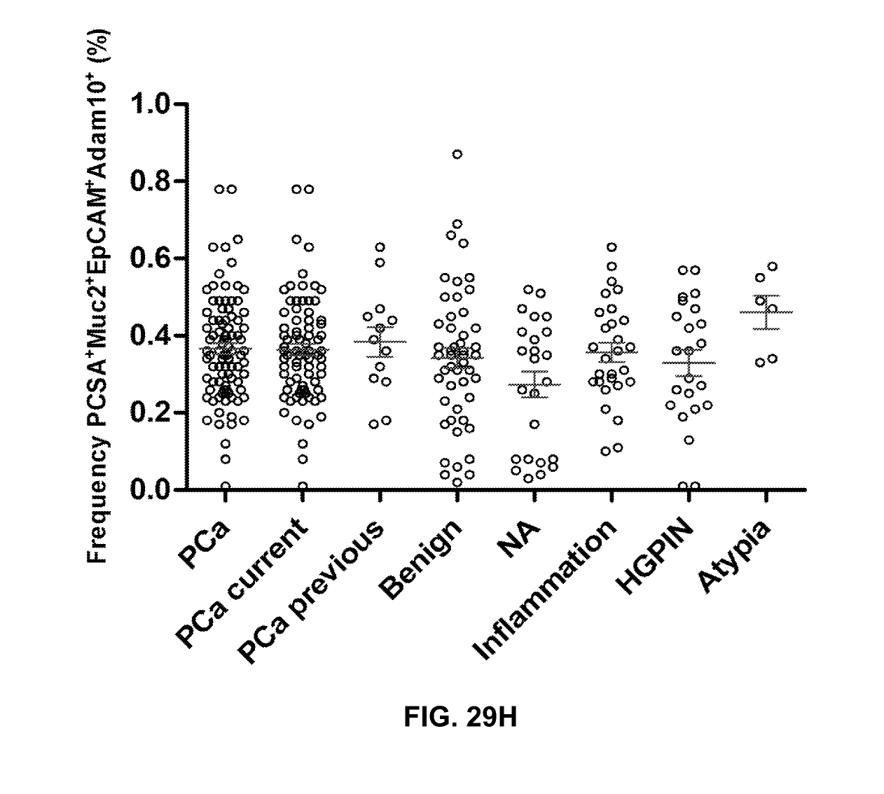

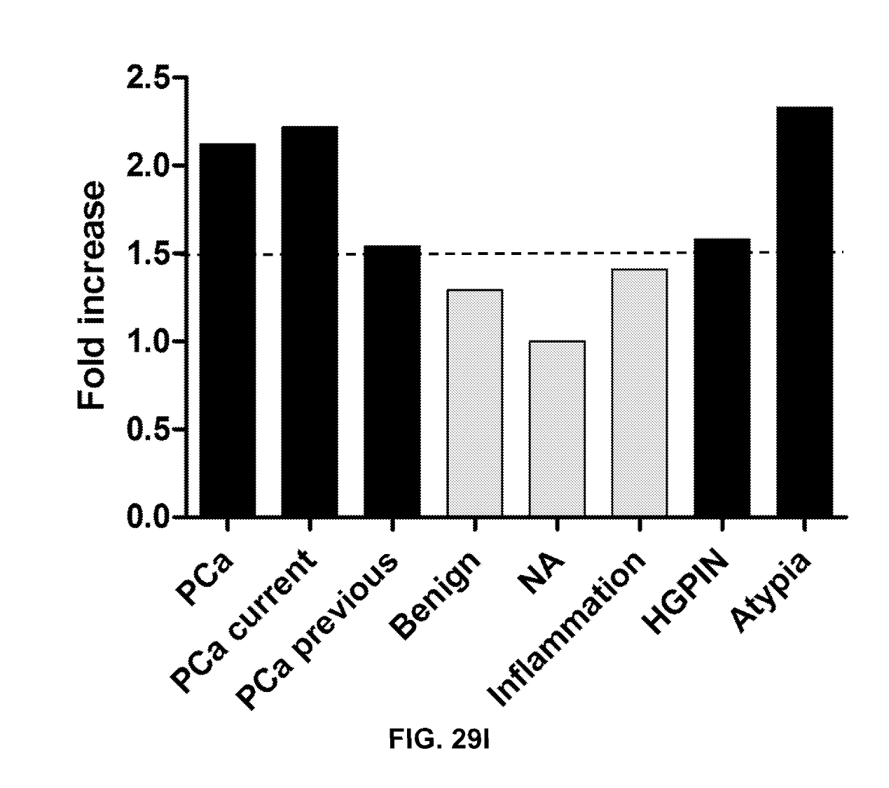

Analysis of microvesicles from plasma samples by a panel of single monoclonal antibodies to EpCAM, PCSA, Muc2 or Adam10 in this cohort showed that biomarkers were expressed with a similar pattern on several types of samples (PCa, Benign, normals, Inflammation, HGPIN, and Atypia). See FIGS. 29A-D. An analysis of different combinations of these four biomarkers co-expressed on microvesicles was also performed. Frequencies of co-expressed markers did not show a significant different between PCa samples and the rest of the cohort, with the exception of Atypia. See FIGS. 29E-H. Atypia samples showed an increased frequency of PCSA+Adam10+ double positive events on EpCAM⁺SSC^{HI}-EpCAM⁺SSC^{LO} ratio (FIG. 29G). Analysis of light side scattering on these microvesicles with EpCAM expression and positive for PCSA-Muc2-Adam10 suggests that cancer samples and HGPIN/Atypia have changed the ratio between these two subpopulations of microvesicles. See FIG. 29I.

Based on previous experiments (see Examples above), four biomarkers, EpCAM, Muc2, Adam10 and PCSA were selected to study the phenotype of plasma microvesicles by flow cytometry. These markers were found to be expressed in similar fashion throughout this cohort. However, analysis of side scatter on triple positive expression of PCSA/Muc2/Adam10 revealed two unique subpopulations based on SSC magnitude and EpCAM expression. These results suggested that different levels of microvesicle complexity could be found in cancer samples with potential prostate cancer diagnosis.

### Example 45

### Differential Protein Expression and miR Content of Sorted Subsets of Circulating Microvesicles from Cancer Patients and Healthy Controls

MicroRNAs (miRs) are small non-coding RNAs that are 20 to 25 nucleotides in length and regulate expression of entire families of genes. Circulating microvesicles (cMV) within biologic fluids are a major source of circulating miRs in cancer patients. The transfer of circulating miRs from diseased cells into the bloodstream and thus remote biological locations can have broad impacts on disease detection, progression and/or prognosis. The goal of these studies was to determine whether there are differences in miR composition within different subpopulations of cMV based on surface protein composition.

We used flow cytometry to phenotype and sort plasma-derived cMV from 20 individuals (3 breast cancer, 2 lung cancer, 6 prostate cancer, 1 bladder cancer and 6 non-cancer controls). cMV were stained for proteins associated with cMV membranes such as tetraspanins (CD9, CD63, and CD81), Ago2 and/or GW182 using a Beckman Coulter MoFlo XDP. For phenotypic analysis, events were gated on tetraspanin expression to distinguish cMV from nano-sized irrelevant debris, and co-expression of GW182 and Ago2 was determined. Quadrant-based sorting was performed for single- and double-positive events. miR content was determined using conventional Taqman probes with the ABI 7900 thermal cycler on extracted RNA from the sorted cMV.

The results of these studies demonstrate that unfractionated cMV were not able to discriminate cancers from non-cancers using miRs-let-7a, -16, -22, -148a or -451 in this population of patients. However, when sorted tetraspanin+, Ago2+ and/or GW182+ populations of cMV were compared between cancer and normal plasma samples, miR expression was generally 5-fold higher in cancer patients than in healthy controls.

These studies demonstrate that cMV can be consistently phenotyped, analyzed and sorted using a flow cytometer and that subpopulations of cMV contain unique miR profiles which can be useful in distinguishing cancer plasma from non-cancer plasma.

### Example 46

### Circulating Microvesicles Contain Elements of the RISC Complex

Circulating microvesicles (cMV) contain microRNAs (miRs), which are short RNA molecules known to regulate gene expression. In cells, miRs bound to an Argonaute (Ago) protein as part of the RNA-Induced Silencing Complex (RISC) are able to regulate mRNA translation. The protein GW182 is a functional partner of Ago, and is another important component of some types of RISC complexes. We investigate here whether microRNA present in cMV are bound to Ago protein as a RISC complex, and whether GW182 is associated with Ago and cMV from human plasma and cultured cells.

Methods:

Whole Microvesicles vs. Lysed Microvesicles Immunoprecipitations (IPs)

Microvesicles were prepared from Vcap, LNcap and 22rv1 cell lines by ultracentrifugation. Microvesicles were measured by BCA and equal total protein amounts were added for both IPs. Magnabind beads with pre-conjugated α-mouse IgG antibody incubated for 1 hour with either α-Ago2 monoclonal antibody (Abcam), α-CD81 monoclonal antibody (BD Biosciences), or α-BrdU monoclonal antibody (Invitrogen) and mouse normal IgG (Santa Cruz) as negative control. Unbound antibodies were washed with PBS+1% BSA. Whole microvesicles or the corresponding microvesicle lysates were added to the beads and incubated for 1 hour at RT.

Whole Microvesicle IP

Beads were washed with mild buffer: PBS pH 7.4+1% BSA.

Lysed Microvesicle IP

Prior to IP reaction, microvesicles were lysed by lysis buffer: 20 mM HEPES pH 7.9, 10 mM NaCl, 1 mM MgCl2, 0.5 M sucrose, 0.2 mM EDTA, 0.5 mM DTT, 0.35% Triton X100 (v/v) and protease inhibitor tablet (1 tablet/50 ml lysis buffer, Roche). After incubation with antibody-bound beads, samples were washed with stringent buffer: Tris-HCl pH 7.5, 1% NP-40, 1% BSA, 1 mM EDTA and 300 mM NaCl.

Ago2 Plasma IP

The microvesicle lysates IP protocol was followed, but neat plasma was used in lieu of lysates. RNA was extracted from all IP methods by TrizolLS (Invitrogen). TaqMan® miR analysis was performed according to the manufacturer (Applied Biosystems).

Results:

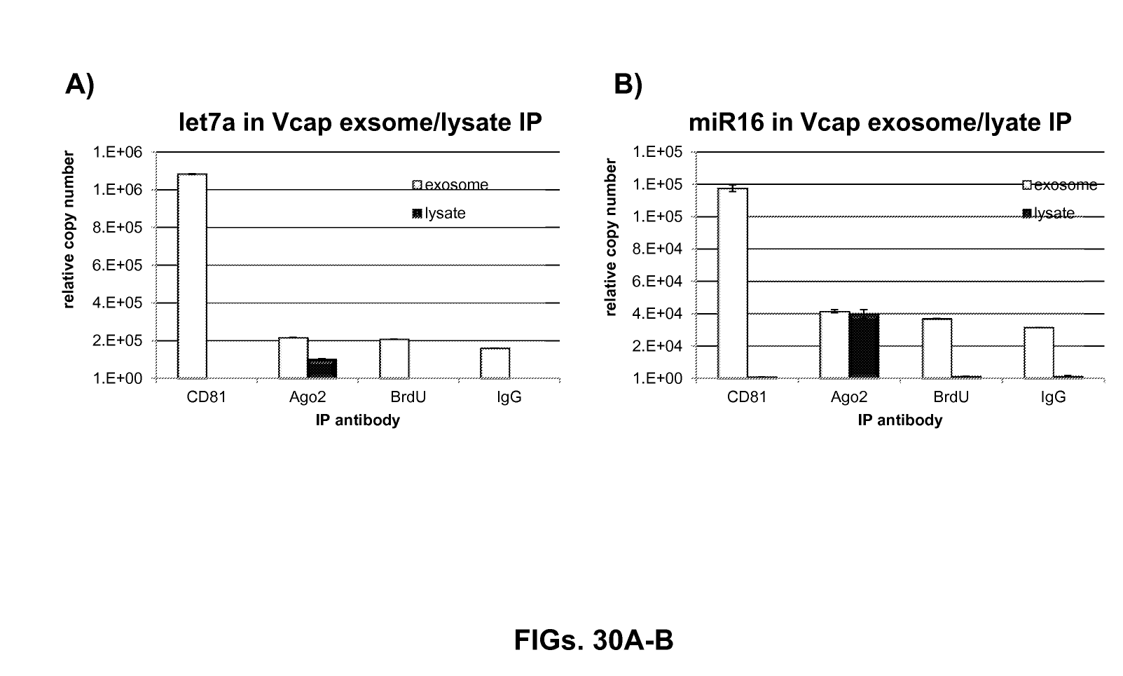

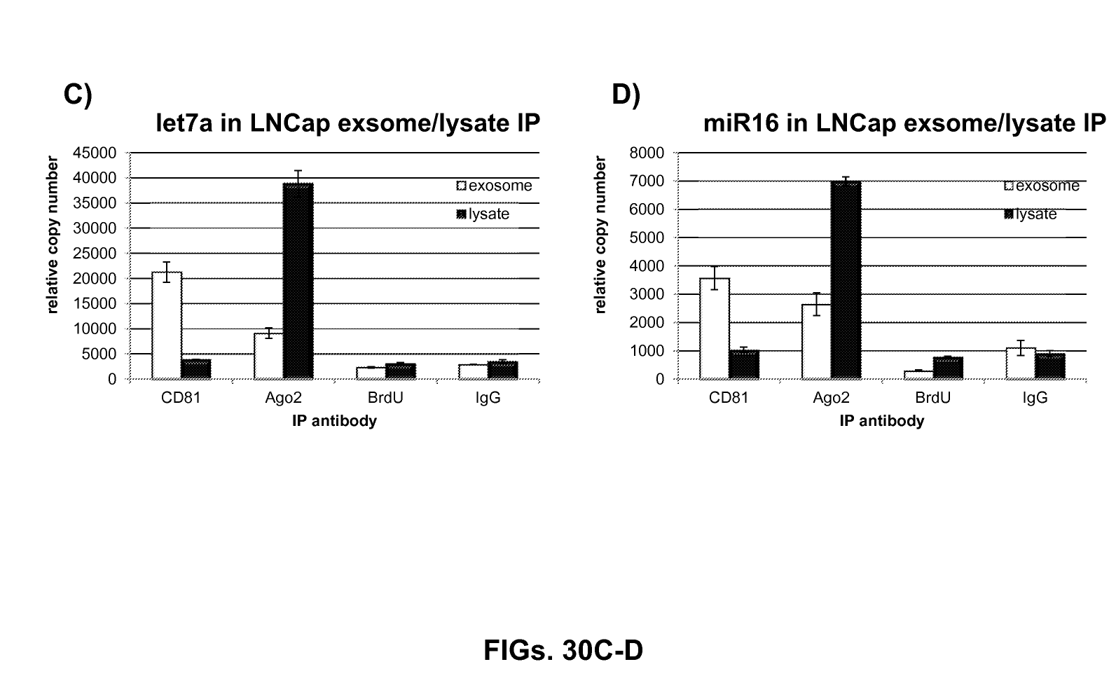

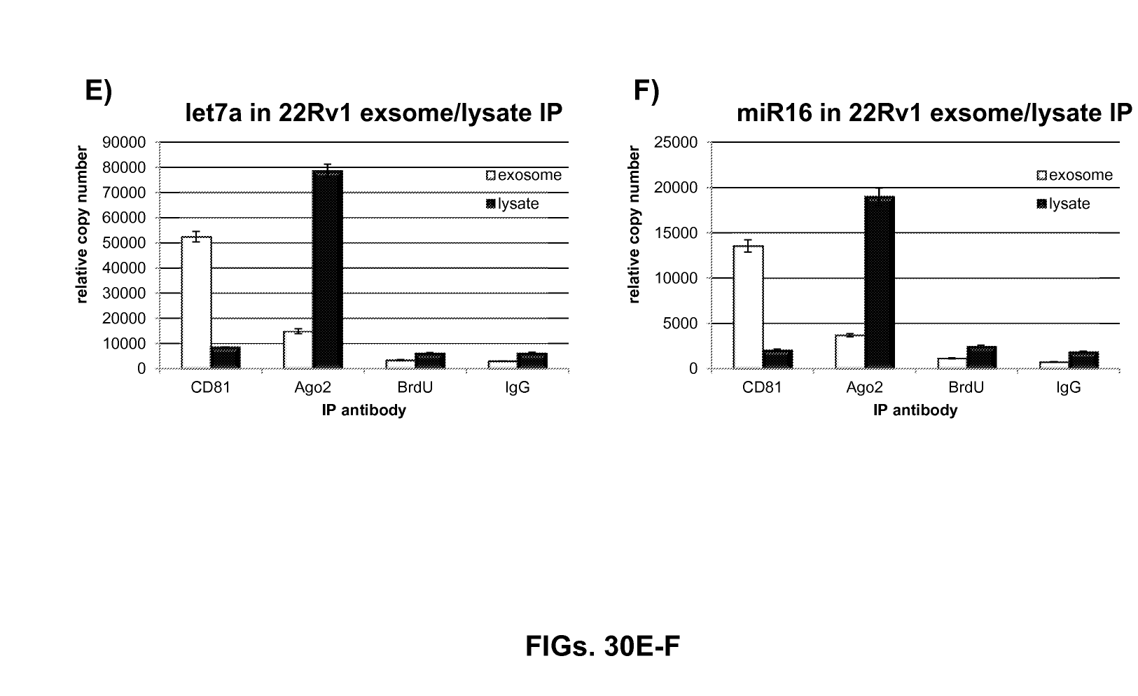

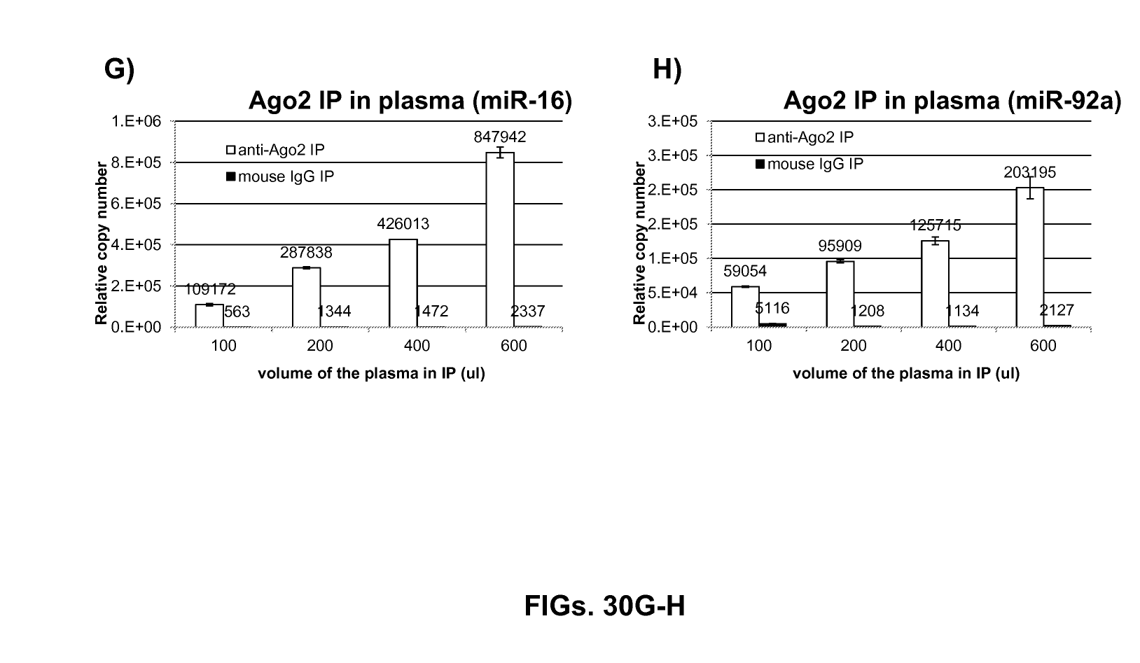

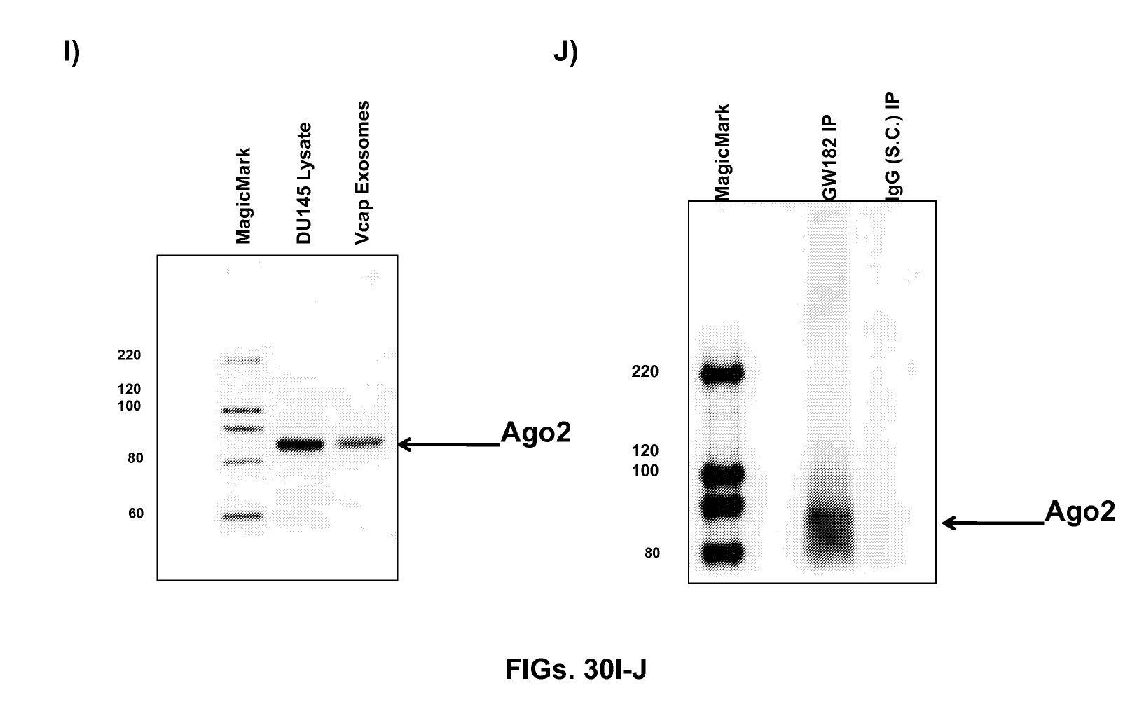

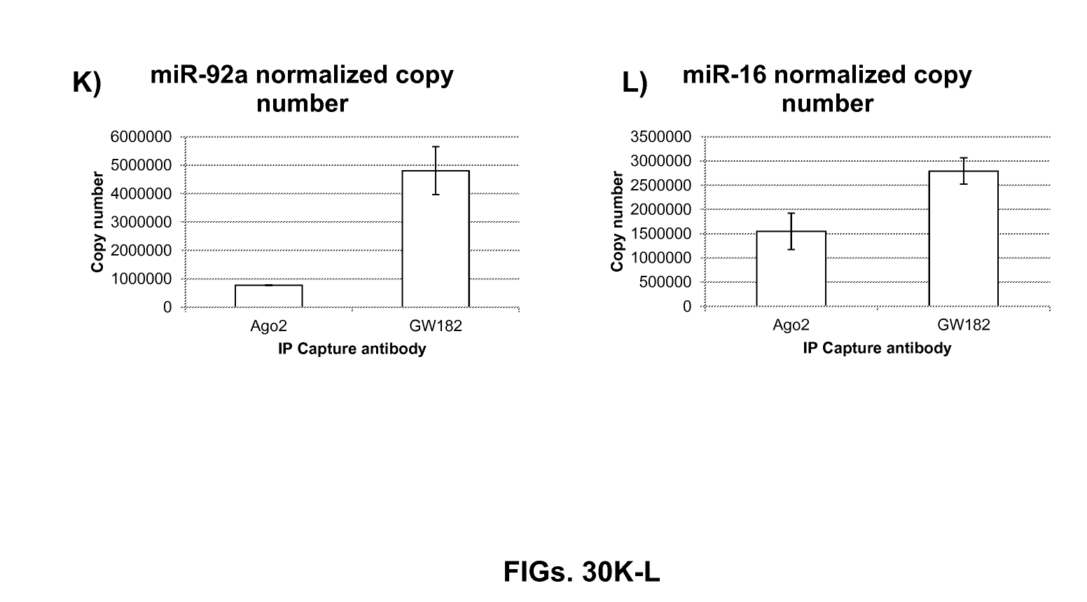

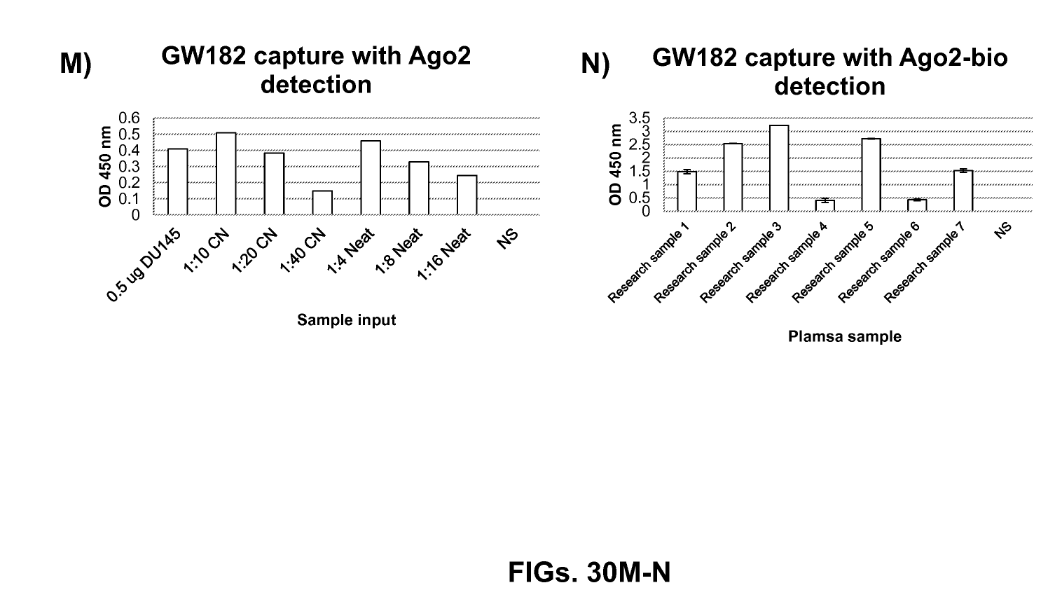

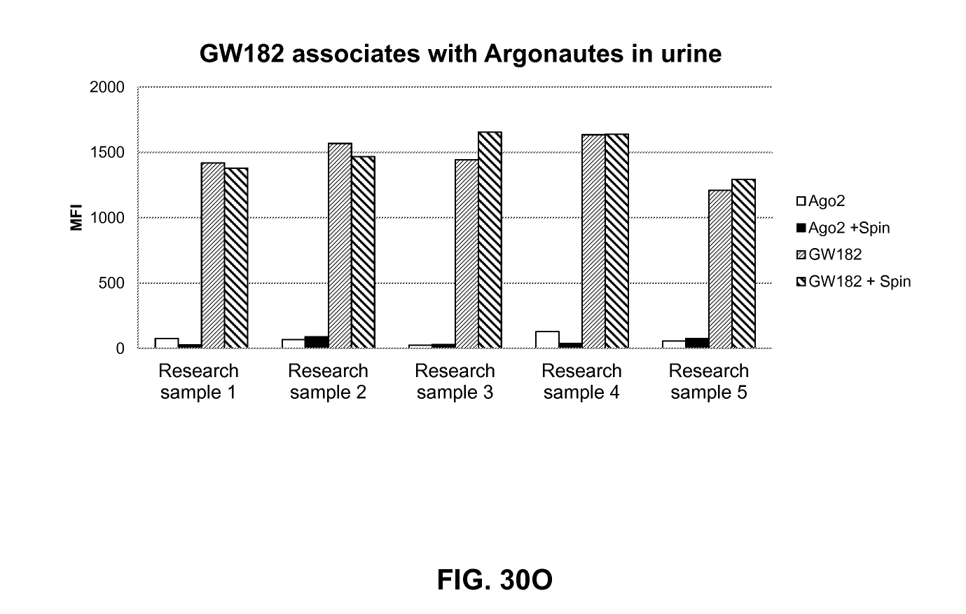

First we investigated whether RISC is present on the outside or inside of cMV. To probe this question, we performed an immunoprecipitation of the proteins Argonaute 2 (Ago2) and CD81 (a cMV-specific marker) from purified cMV from cells in culture. Then, copy numbers for let-7a and miR-16 were determined from anti-Ago2 and anti-CD81 precipitates under both native (i.e., intact cMVs) and lysed cMV conditions. We hypothesize that if Ago2 is bound on the outside of the cMV, then an immunoprecipitation with Ago2 will capture as many miRs as an immunoprecipitation with CD81. However, if Ago2 is bound to miRs on the inside of the cMV, then immunoprecipitation under lysed conditions with Ago2 will capture more miRs than immunoprecipitation under lysed conditions with CD81, and immunoprecipitation under non-lysed conditions with Ago2 will capture fewer miRs than immunoprecipitation under non-lysed conditions with CD81. Microvesicles from three prostate cancer cell lines, VCap, LNCap and 22Rv1, were tested by whole microvesicle IP and microvesicle lysates IP with anti-CD81 (microvesicle surface marker), anti-Ago2, anti-BrdU and mouse normal IgG. Results are shown in FIGS. 30A-F. Anti-CD81 IP with whole microvesicles had greater miR recovery compared to anti-Ago2 IP in all three cell lines; miR recovery using anti-Ago2 antibody is similar to the negative control, indicating the miRs detected were from inside the microvesicles. Anti-CD81 IP with lysed microvesicles showed less miR recovery, while anti-Ago2 IP showed much higher miR recovery compared to anti-CD81, anti-BrdU and mouse normal IgG IPs. Anti-CD81 IP miR recovery is similar to the negative control IP using BrdU antibody and mouse normal IgG, indicating that the microvesicle surface marker CD81 is not a miR-interacting protein, suggesting the Ago2 inside the microvesicle is miR-loaded. These data demonstrates that under non-lysed conditions, the majority of these two miRs were found in the CD81 positive population, with minimal amounts in the Ago2 positive population. However, upon lysis the proportions reversed, and most of the miR was associated with Ago2. These results indicate that these miRs are loaded into Ago2 on the inside of microvesicles. Without being bound by theory, it may be that following exosomal endocytosis, these Ago2-miR complexes will be immediately functional and able to inhibit translation of the complementary mRNA absent any RISC-loading requirements.

The presence of Ago2-miR complexes in plasma was investigated. Ago2 was immunoprecipitated from various volumes of plasma. Mouse normal IgG was used as a negative control. Results are shown in FIGS. 30G-H. Detection of miRs16 (FIG. 30G) and 92a (FIG. 30H) were dependent upon plasma volume input. Large amounts of these miRs were recovered via Ago2 IP, suggesting that Ago2 exists naturally in plasma and is miR-loaded.

Next, we investigated the relationship of GW182 with Ago2 and cMV in human plasma. Antibodies directed toward Ago2 and GW182 were used to immunoprecipitate the proteins from plasma. A Western blot analysis determined that GW182 and Argonaute co-precipitate, suggesting that these two proteins retain their functional relationship in plasma. See FIGS. 30I-J. RNA was then isolated from the immunoprecipitates for miR detection and copy-number analysis. Anti-AGO2 (abcam, ab57113, lot GR29117-1), GW182 (Bethyl Labs, A302-330A) and IgG (Santa Cruz sc-2025) were conjugated to Magnabind protein G beads (Thermo Scientific Cat. #21349). The conjugated beads were incubated with human plasma. RNA was isolated and screened for select microRNAs (miR-16 and miR-92a) using ABI Taqman detection kits (ABI_{—}391 and ABI_{—}431), respectively. RNA was quantified against synthetic standards and normalized to IgG control. Results are shown in FIGS. 30K-L. The GW182-associated miR profile from human plasma contained individual miRs whose abundance either equaled or surpassed that of their matched Ago2 immunoprecipitated miRs. This implies that GW182 maintains an association with the family of Argonaute proteins and a subset of cMV in human plasma.

A sandwich ELISA was used to probe the amount GW182 associated with Ago2 in various human plasma samples. FIG. 30M shows titration of sample input using purified microvesicles (from DU145 cell line) and raw plasma by plate-based ELISA using anti-GW182 as a capture (GW182 (Bethyl Labs, A302-330A) and biotinylated anti-Ago2 (abcam, ab57113, lot GR29117-1) as a detector. In the figure, the signal is normalized to the no sample control. FIG. 30N shows levels of GW182:Ago2 binding in human plasma from seven plasma samples. The signals were normalized to a no sample control. Variable levels of GW182:Ago2 were observed across the plasma samples.

The association of GW182 with Argonautes was then probed in human urine. The relationship between human GW182 and the Argonaute family of proteins was investigated in urine using microbead sandwish assay. GW182 capture was followed by Pan Argonaute detection was tested across five research samples. Results are shown in FIG. 30O. Conditions included raw urine vs cell positive hard spun urine (“+spin” in the figure). As shown, GW182:Ago2 complexes were observed in all samples.

Conclusions:

The presence of Argonaute 2 was confirmed in purified VCaP microvesicles by Western blot. Precipitation of GW182 from human plasma revealed an association with Ago2 by Western analysis. RNA was isolated from samples following IP from human plasma using either anti-Ago2 or anti-GW182. The copy number of known circulating miRNAs was comparable across the IPs.

A plate-based ELISA was developed to evaluate the relationship of GW182 and Argonaute proteins in biological fluids. A signal that titrated with input was observed when GW182 was used as capture followed by Ago2 detection in either raw plasma or concentrated cMV from plasma. Additional research sample were surveyed using the plate ELISA strategy. The levels of GW182:Ago2 positive particles varied dramatically across the sample set. Lastly, an association of GW182 and the Argonaute family of proteins was confirmed across five urine samples using a microbead assay.

GW 182 and Ago2 IP revealed a strong IP of circulating RNA. Both miR-16 and miR-92a were enriched in Ago2 and GW182 IPs. GW182 may be used for the purpose of surveying miRNAs from human plasma and urine. The potential source(s) of miRNA from human plasma and urine include microvesicles/microvesicles and/or circulating Ago2-bound ribonucleoprotein complexes (RNP).

### Example 47

### Lipid Bi-Layer Intercalating Fluorescent Dyes and Expression of Microparticle-Associated Proteins to Detect Microvesicles

Distinguishing true cells from biological debris can be a challenge when performing flow cytometry and may confound analysis. In flow cytometry, laser light is used to evaluate particles in suspension. For cell characterization, the light scattering properties of the particles are evaluated. Forward light scatter is a surrogate for particle size because larger particles refract greater amounts of laser light compared to smaller particles. Side scatter is a surrogate for particle complexity or topography because more complex particles can bounce laser light at more angles. Typically the light scattering properties are forward scatter and side scatter with characteristic properties identified for different cell types. For example, in blood cell characterization, lymphocytes express relatively small forward and side scatter properties compared to monocytes. Cancer and epithelial cells typically express greater forward and side scatter properties than monocytes. In order to evaluate cells and avoid sub-cellular debris, a flow cytometer can be set to analyze particles above a certain size.

Circulating microvesicles (cMVs) in biofluids are smaller than whole cells and have light scattering properties that may indistinguishable from certain biological debris. cMVs are typically considered as a membrane-bound particle between 40-1500 nm in diameter that contains membranous proteins from their cell of origin. These properties can be used together to identify and characterize cMVs using flow cytometry and avoid analyzing debris which is not cMVs.

This Example presents staining and gating strategies to identify and separate cMVs from biological debris using flow cytometry by dual staining with antibodies to cMV proteins and lipid-intercalating dyes to detect membranes. This combination can specifically detect cMV particles as the antibodies and lipids do not have the same non-specific background binding properties.

In this Example, lipid bi-layer intercalating dyes including the long-chain dialkylcarbocyanines DiI and DiO (Invitrogen), and cellular membrane-labeling dyes such as Wheat germ agglutinin-Alexa Fluor 488, were used to identify particles that contain lipid membranes. Lipid intercalating dye FM 1-43 was also evaluated. Similar lipid dyes can be substituted, e.g., if alternate fluorescent properties are required for match multi-parametric analysis (e.g., dialkyl aminostyryl dyes (DiA and its analogs), DiD, DiR). Lipid dyes also may bind to membrane fragments or subcellular organelles such as mitochondria. Thus, the cMVs were also stained for proteins know to be associated with cell plasma membranes; specifically the tetraspanins CD9, CD63 and CD81.

To identify cMVs apart from cellular and other biological debris, a two-stage gating system was used. First, particles detected by the flow cytometer were gated by forward and side light scatter to evaluate particles that are between 40-1500 nm and are relatively small side scatter properties. Second, flourochrome-conjugated protein-specific antibodies and fluorescent lipid-intercalating dyes were used to further characterize the cMVs present.

Fluorescent lipid-intercalating dyes were utilized at the manufacturer's recommended concentration for cells to detect lipid-membranes in cMVs (1 μl of stock dye solution purchased per 200 μl volume). Higher concentrations increased the fluorescent spill-over into other channels which were not able to be compensated for and lower concentrations did not label cMVs efficiently. The concentration of fluorochrome-conjugated antibodies were used as described elsewhere herein. The FITC-labeled anti-tetraspanin antibodies were used at equal concentrations for each component (CD9, CD63, CD81), PE-Cy7-labeled anti-EpCAM antibody was used with a working stock solution of 83.33 μg/ml, and PE-Cy7-labeled anti-EGFR antibody at 92.3 μg/ml.

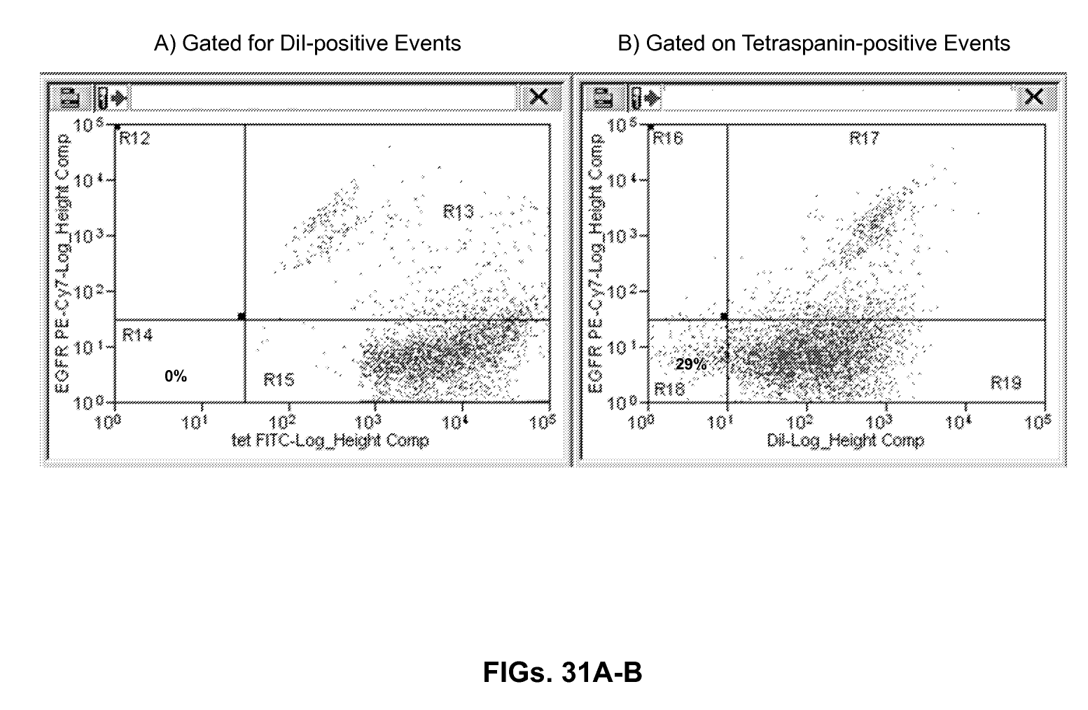

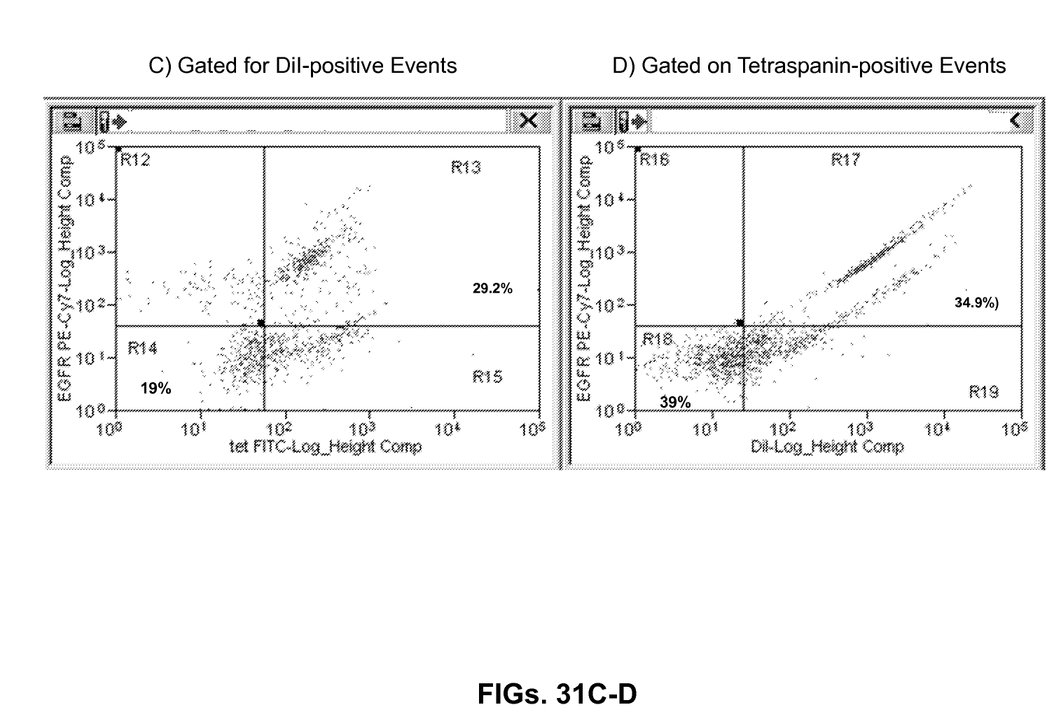

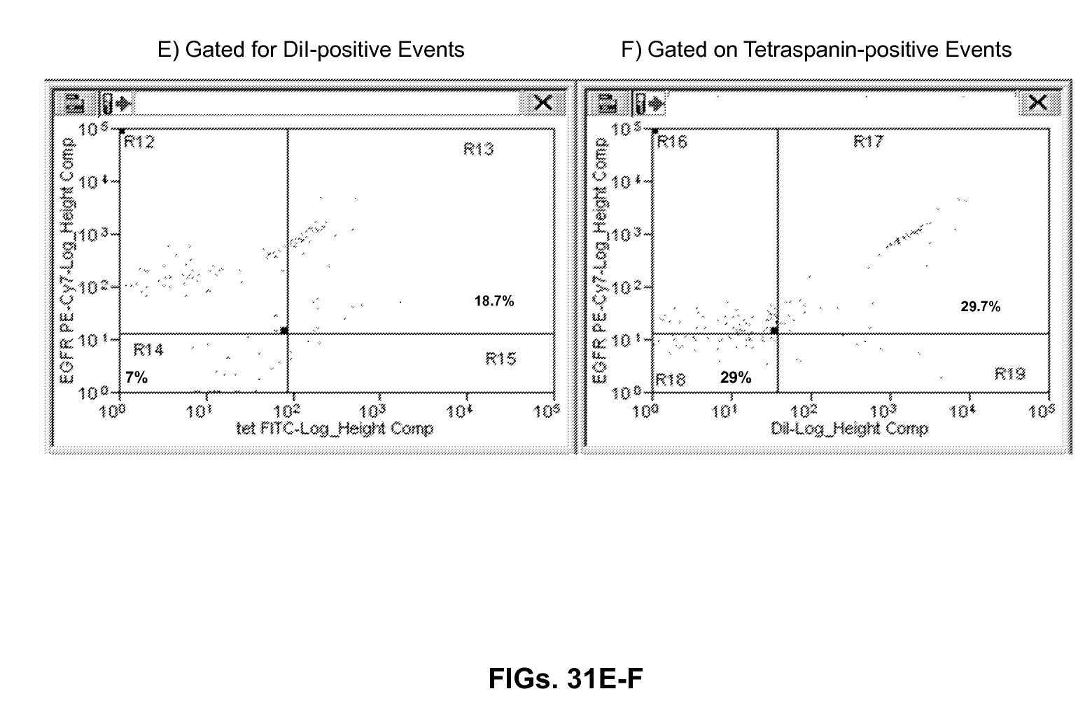

To examine whether the antibodies or the dye may be physically hindering blocking sites of the other components, samples were dyed before, during, or after staining with the various antibodies. Using vesicles isolated from VCaP cell culture using ultrafiltration as described herein, gating on DiI-positive events reduced tetraspanin+/EGFR+ double-negative events (i.e., events considered to correspond to debris) to nearly zero, no matter in what order the cMVs were stained. See, e.g., FIGS. 31A-F. In FIG. 31A, the vesicles were first gated for DiI+ events then EGFR+/tetraspanin+ events were counted. As indicated, 0% double negative events corresponding to cellular debris were observed. In FIG. 31B, the vesicles were first gated for tetraspanin+ events then EGFR+/DiI+ events were counted. As indicated, 29% double negative events corresponding to cellular debris were observed. FIG. 31C and FIG. 31D illustrate staining of vesicles concentrated from plasma of cancer-positive patients. Experimental conditions were otherwise identical to FIG. 31A and FIG. 31B, respectively. FIG. 31E and FIG. 31F illustrate staining of vesicles concentrated from plasma of cancer-negative patients. Experimental conditions were otherwise identical to FIG. 31A and FIG. 31B, respectively. For tetraspanin+ gated events, staining with the anti-tetraspanin antibody cocktail prior to adding dye reduced DiI+/EGFR+ double-negative events to 6%, compared to 30-40% in the other staining conditions. Gating on DiI-positive events similarly reduced tetraspanin+/EpCAM+ double-negative (debris) events to nearly zero. In sum, the particular gating strategies did not significantly alter the results with VCaP vesicles although initial gating on DiI-positive events yielded optimal results.

Circulating microvesicles (cMVs) in biofluids were investigated next. Vesicles from patient plasma samples were isolated using ultrafiltration as described herein. Vesicles concentrated from patient plasma samples have a much higher degree of debris overall that those isolated from cell lines. For vesicles concentrated from a pool of cancer-positive plasma samples, gating on DiI reduced tetraspanin+/EGFR+ double-negative events (i.e., events considered to correspond to debris) when stained with DiI dye and the anti-tetraspanin/anti-EGFR antibodies simultaneously, or when the cMVs were first stained with the antibodies followed by the dye. DiI+ gating also reduced the non-specific events in all staining conditions, compared to gating on tetraspanin+ events. Similar differences in populations were observed when tetraspanin+/EpCAM+ events were first gated for DiI+ events. When examining vesicles concentrated from a pool of cancer-negative plasma samples which have a lower concentration of cMVs that the cancer positive pool, gating on DiI reduced tetraspanin+/EGFR+ double-negative (debris) events except when stained with dye first. A similar reduction was seen in tetraspanin+/EpCAM+ double-negative events. Also, gating on DiI-positive events reduced the non-specific 45° events in all staining conditions, compared to gating on tetraspanin+ events alone.

Various experimental conditions were tested. For example, the above experiments were repeated with 0.01% polysorbate 20 (commercially available as Tween® 20 from various vendors). Results were similar to the above. Increasing concentration of DiI (1×, 2×, 5× concentrations) as well as increased incubtation time (to 2-3 h) with DiI prior to gating were also tested. In both cases, the DiI signal increased at the expense of higher levels of background staining which may results in false positives.

Taking together the results from above, a reliable approach to separate cMVs from biological debris appeared to be staining cMVs simultaneously with the lipid dye and binding agents to vesicle protein markers, followed by gating for the lipid dye positive events to identify lipid-positive particles, then detection of protein+cMVs. Double gating of lipid containing microparticles that also express common cMV antigens (e.g., tetraspanins) is another possibility.

References:

Tsien, R. Y., Ernst, L. and Waggoner, A., Fluorophores for Confocal Microscopy: Photophysics and Photochemistry. Handbook of Biological Confocal Microscopy, 3rd Edition, 2006, James B. Fawley Editor, Springer Science+Business Media, NY. pp. 338-352; Bolte et. al., FM-dyes as experimental probes for dissecting vesicle trafficking in living plant cells. 2004. J. Microscopy, 214(pt2):159-73; Sengupta et. al., Fluorescence resonance energy transfer between lipid probes detects nanoscopic heterogeneity in the plasma membranes of live cells. 2007. Biophysical Journal 92:3564-74.

### Example 48

### Detecting Microvesicles Using an Esterase-Activated Lipophilic Dye

The Example above demonstrated detection of microvesicles using lipid dyes. In this Example, microvesicles are stained with lipophilic dyes and then used to determine microvesicle concentration in a biological sample.

Overview:

A standard curve is created with different concentrations of microvesicles isolated from human plasma samples with concentration obtain by flow cytometry. One ml of one plasma sample is pooled with samples from other patients to create a sample pool. The sample pools and the test samples are subjected to thromoboplastin treatment and the Exoquick kit is used to isolate microvesicles. Five dilutions from 3 to 0.1875 million events per μl are prepared and stained accordingly to the protocol below to create a standard curve. Test samples with unknown microvesicle concentration are then stained with carboxyfluorescein succinimidyl ester (CFDA) dye. Microvesicle associated esterases will convert the CFDA to carboxyfluorescein succinimidyl ester (CFSE), which can be detected using a fluorescence reader. The fluorescence readings are interpolated into the standard curve to obtain their microvesicles concentration. Standard curve and test samples are incubated with CFSE at a final concentration up to 480 μM per well. After 15 min incubation the plate is read on the qRT-PCR instrument model ViiA™ 7 system (Life Technologies Corporation, Carlsbad, Calif.) to record fluorescence intensity. The method allows microvesicle concentration to be quickly determined using a fluorescence reader.

Reagents:

Carboxyfluorescein succinimidyl ester (CFSE). Fluorescent form.

Carboxyfluorescein diacetate succinimidyl ester (CFDA). Non-fluorescent precursor of CFSE which can become fluorescent when esterases remove the acetate groups.

VYBRANT CFDA SE CELL TRACER KIT (Invitrogen/Life Technologies; Catalog Item V12883): This kit contains DMSO and 10 vials of CFSE. Add 90 μl of DMSO to one vial of CFSE and mix. This stock is 10 mM. Prepare a 960 μM dilution from this to use for the experiment. (25 μl needed per well). Keep covered in dark.

ExoQuick Exosome Precipitation Solution (System BioSciences, Inc., Mountain View, Calif.; Catalog Item EXOQ20A-1)

Thromboplastin-D (System BioSciences, Inc., Mountain View, Calif.; Catalog Item 100357)

Phosphate buffered saline (PBS), sterile water

Equipment and Supplies:

Plates—MicroAmp qPCR plates 96 well with barcode (Invitrogen/Life Technologies)

RT-PCR Instrument—ViiA™ 7 (Applied Biosystems/Life Technologies)

Sample Preparation:

Prepare sample pools by mixing several 100 μl aliquots of frozen plasma samples.

Choose 100 μl aliquots of test plasma samples for which the exosome concentrations are to be determined.

For the pooled and test samples, perform double fibrinogen depletion using Thromboplastin-D and perform ExoQuick to isolate vesicles according to manufacturer's instructions.

Resuspend pellet from the Exoquick protocol of sample pool in water (use half of initial pool volume).

Resuspend pellet of test samples in water in initial volume (100 μl).

Flow Cytometry Reading:

Take 1 μl of the sample pool and resuspend in 299 μl of sterile 0.1 μm filtered PBS and run the sample on the flow machine (machine settings 19.5 μl/min, aspiration 150 μl, 90 secs). Run in triplicate.

Obtain Gate 8 events from the data files and multiply by 10 to give events/μl of sample pool. Determine average number of events. (Test samples are not counted).

Standard Curve:

Aliquot required volume of sample pool pellet for 6 million events and bring it to a final volume of 50 μL Pipet into the first well of a clean MicroAmp plate.

Add 25 μl of PBS into 4 wells following the first well. Serially dilute into these wells using 25 μl from the first well, ending up in final volume of 25 μl in all 5 wells.

Add 10 μl of the vesicle pellets of the two test samples to two different wells and bring volume to match with pooled standards (25 μl).

Add 25 μl of 960 μM CFSE dye to all 5 wells of standards and two wells of test samples. Total volume in each well is now 50 μL

Incubate the plate at 37° C., for 15 min in dark.

Read plate on the ViiA7:

a. Open ViiA7 software

b. Create new experiment using the appropriate template.

c. Choose SYBR assay and the 96 well plate (0.1 ml) option

d. Choose how many samples to be read and select the wells by clicking on each sample.

e. Drag across all wells and select appropriate template

f. Select to run cycle for 20 runs, with minimum hold time ˜2 secs

g. Click on “Start run” and wait for 2 minutes until run is finished

h. Export data to spreadsheet.

i. Analyze using “Multicomponent” tab from exported spreadsheet

j. Interpolate numbers for unknown samples from standard curve fluorescence.

Results:

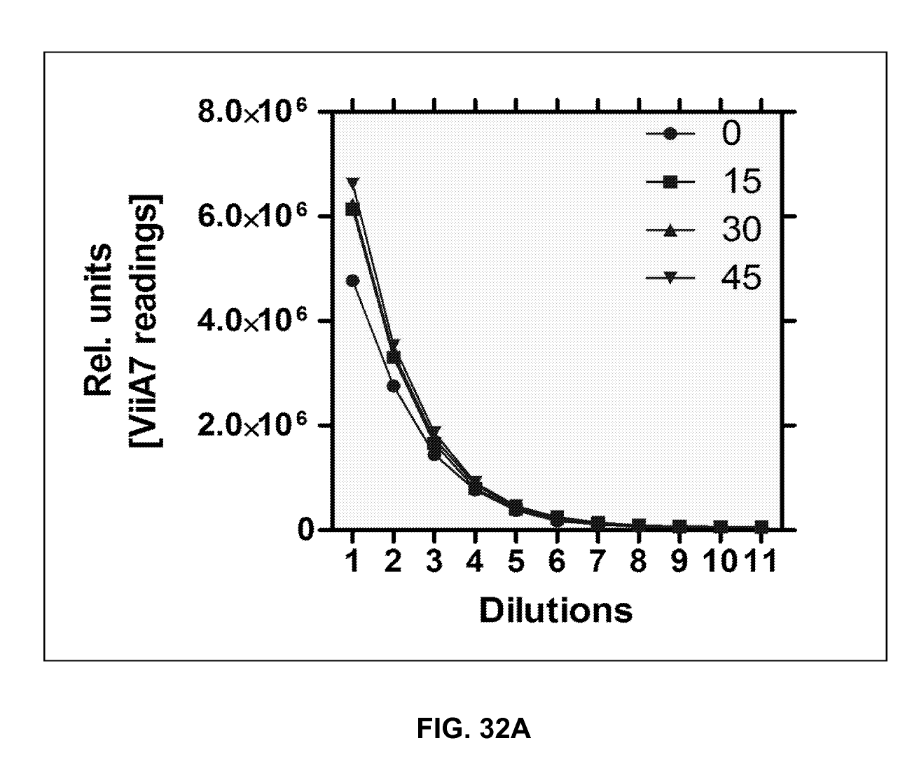

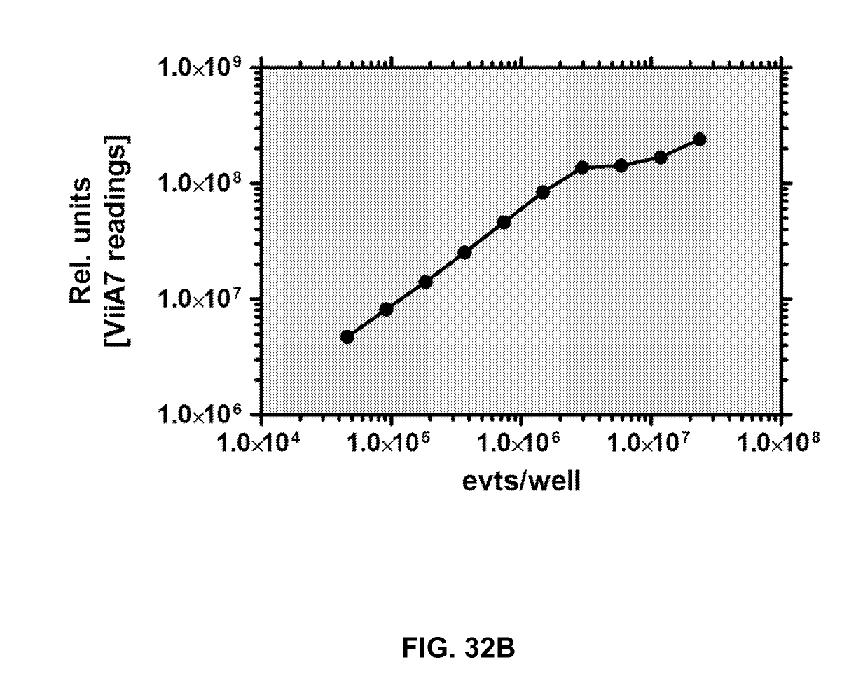

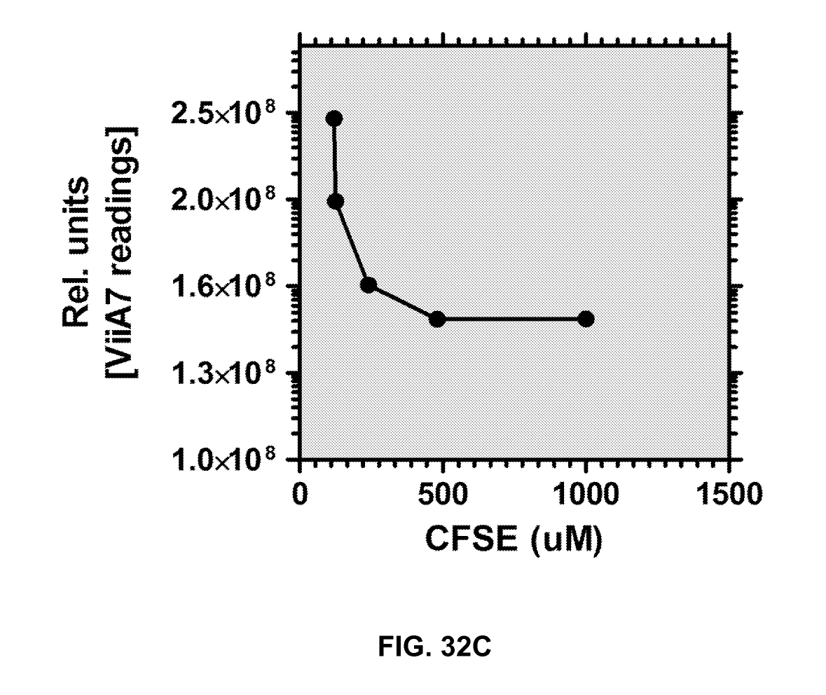

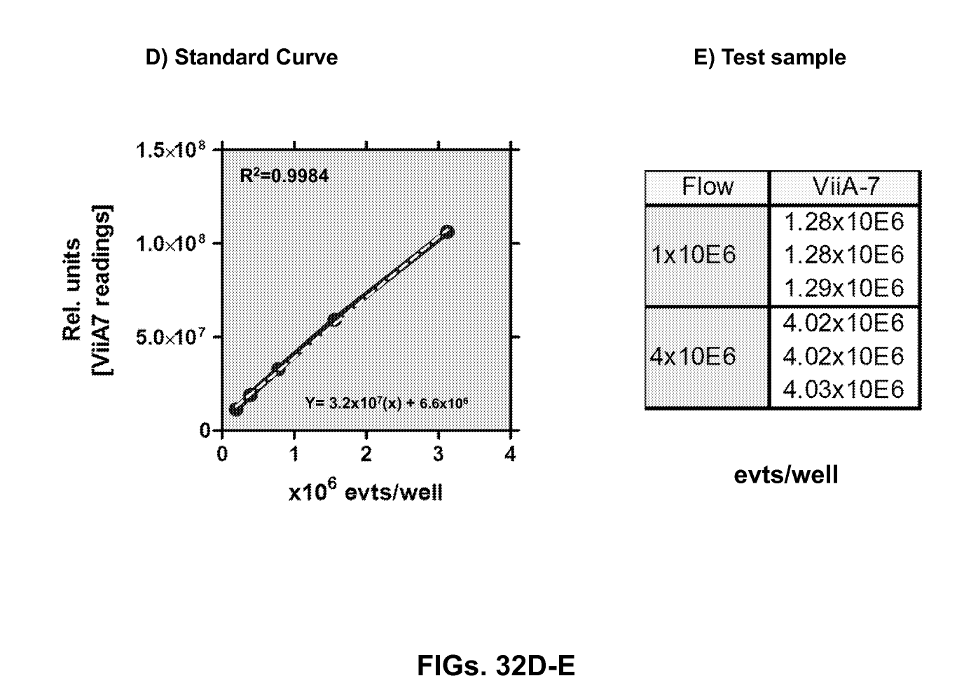

The above protocol was performed to generate a standard curve for estimating a microvesicle concentration in an unknown test sample. FIG. 32A shows serial dilution of vesicles stained with 40 μM of CFSE according to vendor instructions. After staining, the vesicles were serially diluted 11 times (see X axis) and fluorescence was detected coming from the conversion of non-fluorescent dye to its fluorescent ester form after microvesicle esterases remove the acetate groups (see Y axis). CFSE fluorescence was determined at several time-points (0, 15, 30 and 45 min post incubation, as indicated in the figure) to evaluate enzymatic activity over time. The CFSE fluorescent signal was consistent after 15 min of incubation and fluorescence values correleated to microvesicle concentration. Readings from negative control (sample without CFSE) or positive control (CFSE without microvesicles) were very low, indicating that autofluorescence or inactive CFSE does not significantly contribute to the detected fluorescence signal (data not shown).

FIG. 32B shows a standard curve generated using CFSE stained microvesicles. 50×10⁶ microvesicles as determined using flow cytometry were stained with 40 μM in 400 μl to create the standard curve. The curve was generated by detecting fluorescence in a series of dilutions using a Viaa7 RT-PCR machine as described above. FIG. 32C shows the effects of CFSE concentration (μM) on microvesicle staining. The signal plateaued at ˜480 μM, indicating that the test samples and standard curve stained closer to 480 μM should minimize staining variation and signal will be due to cMV concentration.

FIG. 32D and FIG. 32E illustrate determination of microvesicle concentration in a test sample using a standard curve. The protocol is outlined in detail above. In these experiments, the standard curve samples and test samples were stained with 370 μM CFSE then incubated at room temperature before they were loaded on 96-well (MicroAmp) plate. In FIG. 32D, fluorescence relative units (Y-axis, Viia-7 system readings) were plotted against microvesicle concentration (X-axis). Linear regression was used to calculate a standard curve as shown in the plot. Based on the regression, two test samples of known concentration as determined by flow cytometry were stained with 370 μM CFSE and fluorescence was determined using the ViiA-7 system. Fluorescence values were interpolated to the standard curve to determine microvesicle concentration in the test samples. As seen in the table in FIG. 32E, determination of the concentration of microvesicles stained with CFSE dye agreed well with the flow cytometry data. Similar results were obtained using 480 μM CFSE to stain the microvesicles. When test samples were analyzed in triplicate, intersample CV % was lower when the sample was first stained and then aliquoted (CV=2.4%) versus when the sample was first aliquoted then stained (CV=15.33%). However, both methods yielded acceptable results.

Taken together, these data indicate that microvesicles can be reliably stained with CFDA, which will be converted to CFSE, and detected using a fluorescence plate reader. These data further demonstrate that a standard curve can be generated using CFSE stained microvesicles in order to determine a microvesicle concentration in a test sample.

### Example 49

### Identification of DNA Oligonucleotides that Bind a Target

The target is affixed to a solid substrate, such as a glass slide or a magnetic bead. For a magnetic bead preparation, beads are incubated with a concentration of target protein ranging from 0.1 to 1 mg/ml. The target protein is conjugated to the beads according to a chemistry provided by the particular bead manufacturer. Typically, this involves coupling via an N-hydroxysuccinimide (NHS) functional group process. Unoccupied NHS groups are rendered inactive following conjugation with the target.

Randomly generated oligonucleotides (oligos) of a certain length, such as 32 base pairs long, are added to a container holding the stabilized target. Each oligo contains 6 thymine nucleotides (a “thymine tail”) at either the 5 or 3 prime end, along with a single molecule of biotin conjugated to the thymine tail. Additional molecules of biotin could be added. Each oligo is also manufactured with a short stretch of nucleotides on each end (5-10 base pairs long) corresponding to amplification primers for PCR (“primer tails”).

The oligonucleotides are incubated with the target at a specified temperature and time in phosphate-buffered saline (PBS) at 37 degrees Celsius in 500 microliter reaction volume.

The target/oligo combination is washed 1-10 times with buffer to remove unbound oligo. The number of washes increases with each repetition of the process (as noted below).

The oligos bound to the target are eluted using a buffer containing a chaotropic agent such as 7 M urea or 1% SDS and collected using the biotin tag. The oligos are amplified using the polymerase chain reaction using primers specific to 5′ and 3′ sequences added to the randomized region of the oligos. The amplified oligos are added to the target again for another round of selection. This process is repeated as necessary to observe binding enrichment.

### Example 50

### Competitive Assay

The process is performed as in Example 49 above, except that a known ligand to the target, such as an antibody, is used to elute the bound oligo species (as opposed to or in addition to the chaotropic agent). In this case, anti-EpCAM antibody from Santa Cruz Biotechnology, Inc. was used to elute the aptamers from the target EpCAM.

### Example 51

### Tripartite Aptamer and Target Binding Optimization

Cancer may induce immunosuppression in the host as a biologic mechanism to evade immune destruction. The mechanisms of immunosuppression can be highly diverse and impact all arms of the immune system; innate, adaptive, cellular and humoral. Common cellular targets for immunosuppression by cancer include dendritic cells, monocytes, macrophages, NK cells, NKT cells, gammadelta T cells, alphabeta T cells (both CD8 killer cells and CD4 helper cells) and B-cells. Any and sometimes all of these cells have been found to be deficient in various cancers, particularly of an advanced stage.

A common immunosuppression mechanism involves tumor-derived factors that can be either secreted freely into the surrounding tumor microenvironment or in association with microvesicles. Such immunosuppressive factors can include membrane proteins like CD39 or CD73, cytokines like IL-10 and TGF-β or apoptosis-inducing molecules like FasL or TRAIL.

This Example addresses the problem of reducing the immunosuppression of cancer by inhibiting the immunosuppressive factors produced by the cancer cells both at their source and when associated with microvesicles. With antibody therapy, the host often develops anti-idiotypic antibodies rendering the antibody therapy less effective or an alternate immunosuppressive pathway compensates for the blocked factor. This Example provides a therapeutic agent that configured to bind to tumor-derived circulating microvesicles (cMVs), block one or more immunosuppressive factor on the CMVs, and also stimulate the interacting immune cell to resist other immunosuppressive factors and support or induce anti-tumor immunity. Because cMVs closely resemble their cell of origin regarding membrane structure, the therapeutic agent may also bind to the tumor cells which will have a synergistic impact.

The invention is comprised of a three component synthetic DNA oligonucleotide aptamer composed of: 1) a binding site for a cancer cell specific protein, 2) a binding site for an immunosuppressive tumor-derived protein found on cMVs and cancer cells and 3) an immune-modulatory oligonucleotide linker arm between these two components. See FIGS. 33A-33B. The cancer specific target protein may consist of a membrane-associated protein prevalent on vesicles shed by various types of cancer or restricted to a specific cancer type. The immunosuppressive target protein can include without limitation TGF-β, CD39, CD73, IL10, FasL or TRAIL. The oligonucleotide linker sequence might include TLR agonists like CpG sequences which are immunostimulatory and/or polyG sequences which can be anti-proliferative or pro-apoptotic. The trivalent aptamer may bind both tumor-derived cMVs as well as tumor cells in the treated patient for an enhanced effect.

Synthesis and screening of each of the binding components of the trivalent aptamer are determined individually. See, e.g., Example 49 and discussion above, particularly concerning SELEX methodology. Candidate aptamers are confirmed using binding assays for target protein and further for physiological effects on immunomodulation in cell culture. Binding is confirmed using surface plasmon resonance (SPR) or isothermal titration calorimetry (DSC). Selection of the individual DNA oligonucleotide aptamers uses previously published protocols (Nadal et al., 2011).

Exemplary sequences for each region of the trivalent aptamer are shown in Table 47:

TABLE 47
Immunomodulatory and Anti-proliferative Regions of a
Trivalent Aptamer

| | | SEQ ID |
|---|---|---|
| Region | Sequence 5′->3′ | NO. |
| CpG regions | TCCATGACGTTCCTGATCT | 2 |
| | GCTAGACGTTAGCGT | 3 |
| | ATCGACTCTCGAGCGTTCTC | 4 |
| Poly G | GGTTGGTGTGGTTGG | 5 |
| regions | GGGGTTTTGGGGTTTTGGGGTTTTGGGG | 6 |
| | TTGGGGTTGGGGTTGGGGTTGGGG | 7 |
| | GGTTTTGGTTTTGGTTTTGG | 8 |
| | GGGGTTGGGGTGTGGGGTTGGGG | 9 |
| | TTTGGTGGTGGTGGTTGTGGTGGTGGTGG | 10 |
| Hybrid CpG- | GGTTGGTTCCATGACGTTCCTGATCTGTGGTTGG | 11 |
| Poly G | GGGGTTTTGGTCCATGACGTTCCTGATCTGGTTTTGGGGTTTTGGGG | 12 |
| nucleotides | TTGGGGTTGGTCCATGACGTTCCTGATCTGGTTGGGGTTGGGG | 13 |
| | GGTTTTGTCCATGACGTTCCTGATCTGTTTTGGTTTTGG | 14 |
| | GGGGTTGGGGTGTGGTCCATGACGTTCCTGATCTGGTTGGGG | 15 |
| | TTTGGTGGTGTCCATGACGTTCCTGATCTGTGGTTGTGGTGGTGGTGG | 16 |
| | GGTTGGTGCTAGACGTTAGCGTGTGGTTGG | 17 |
| | GGGGTTTTGGGCTAGACGTTAGCGTGGTTTTGGGGTTTTGGGG | 18 |
| | TTGGGGTTGGGCTAGACGTTAGCGTGGTTGGGGTTGGGG | 19 |
| | GGTTTTGGCTAGACGTTAGCGTGTTTTGGTTTTGG | 20 |
| | GGGGTTGGGGTGTGGGCTAGACGTTAGCGTGGTTGGGG | 21 |
| | TTTGGTGGTGGCTAGACGTTAGCGTGTGGTTGTGGTGGTGGTGG | 22 |
| | GGTTGGTATCGACTCTCGAGCGTTCTCGTGGTTGG | 23 |
| | GGGGTTTTGGATCGACTCTCGAGCGTTCTCGGTTTTGGGGTTTTGGGG | 24 |
| | TTGGGGTTGGATCGACTCTCGAGCGTTCTCGGTTGGGGTTGGGG | 25 |
| | GGTTTTGATCGACTCTCGAGCGTTCTCGTTTTGGTTTTGG | 26 |
| | GGGGTTGGGGTGTGGATCGACTCTCGAGCGTTCTCGGTTGGGG | 27 |
| | TTTGGTGGTGATCGACTCTCGAGCGTTCTCGTGGTTGTGGTGGTGGTGG | 28 |

CpG region sequences in Table 47 are gleaned from Klinman et al. 1996. Poly G region sequences in Table 47 are gleaned from Dapic et al, 2003. These references are incorporated by reference herein in their entirety.

Multiple cycles of SELEX protocols are used for oligonucleotide selection from a pool of 10¹⁵ random single stranded DNA oligonucliotide sequences with confirmation of binding using SPR to the target proteins. See Nadal et al., 2011 for further details on methodology.

### Example 52

### Tripartite Aptamer Linker Optimization

The tripartite aptamer above is optimized as follows.

Select the Immunomodulatory Linker

In vitro studies are used to select and optimize the immunomodulatory linker arm taking into consideration the intended target cells (e.g., immune cells) and potential off target cells (e.g., cancer cells). In this Example, the linker is optimized for intended target immune cells and off target prostate cancer cells. Murine prostate cancer cell lines including TRAMP-C1 (transgenic adenocarcinoma of mouse prostate-C1) are used. Syngeneic (C57BL/6) immune cell lines are selected to facilitate a co-culture model system using multi-lineage mouse splenocytes and prostate tumor cells. Oligonucleotides containing various amounts of CpG motifs (generally considered immunostimulatory) and polyG sequences (anti-proliferative and/or pro-apoptotic) are generated and evaluated using in vitro cell culture models. CpG activates mammalian B cells, natural killer (NK) cells, monocytes/dendritic cells (DCs) and possibly certain T cells. PolyG sequences tend to block IFN secretion as well as downstream effects from CpG stimulation. PolyG sequences may further block cell proliferation, cell motility and invasion. These effects may be beneficial if prostate tumor cells are inadvertently stimulated by CpG sequences in the linker arm. PolyG sequences may form complex and stable tertiary structures including G-quartet which may increase cellular uptake independent of Toll-Like Receptors (TLRs) which could stimulate prostate cancer cells to divide or metastasize but also activate beneficial immune cells, e.g., NK cells.

Procedures for Cell Culture of Cell Lines and Primary Cells:

The following procedures are used for propagation of TRAMP-CI and C57BL/6 spleen cells.

Culture media for cells:

ATCC complete growth medium: Dulbecco's modified Eagle's medium with 4 mM L-glutamine adjusted to contain 1.5 g/L sodium bicarbonate and 4.5 g/L glucose supplemented with 0.005 mg/ml bovine insulin and 10 nM dehydroisoandrosterone, 90%; fetal bovine serum, 5%; Nu-Serum IV, 5%. Atmosphere: air, 95%; 5% carbon dioxide (CO2). T=37.0° C.

Subculturing Protocol:

Remove and discard culture medium. Briefly rinse the cell layer with 0.25% (w/v) Trypsin-0.53 mM EDTA solution to remove all traces of serum that contains trypsin inhibitor. Add 2.0 to 3.0 ml of Trypsin-EDTA solution to flask and observe cells under an inverted microscope until cell layer is dispersed (usually within 5 to 15 minutes).

To avoid clumping do not agitate the cells by hitting or shaking the flask while waiting for the cells to detach. Cells that are difficult to detach may be placed at 37° C. to facilitate dispersal. Add 6.0 to 8.0 ml of complete growth medium and aspirate cells by gently pipetting. Add appropriate aliquots of the cell suspension to new culture vessels. Incubate cultures at 37° C.

Subcultivation Ratio: A subcultivation ratio of 1:6 to 1:10 is recommended

Medium Renewal: Two to three times weekly.

Evaluate Presence of TLRs on Target Cells Types

The presence of TLRs is evaluated on cells of the in vitro co-culture model including tumor cells, monocyte, T cell and B cell lines using flow cytometry. TLRs which are expected to interact with the immunomodulatory linker include TLR7, 8 and 9 but other TLRs are evaluated using flow cytometry with labeled antibodies.

Disruption and digestion of mouse spleens for cells for detection of TLRs before and after linker oligonucleotide exposure uses the tissue disruption protocol as described by Krill et al., 1997 and flow cytometry staining for the indicated antigens described below.

Miltenyi Magnetic Bead Separation of Spleen Cell Subtypes

Miltenyi Biotec's MACS System (Miltenyi Biotec Inc., Auburn, Calif., USA) is used according to the manufacturer's protocols for mouse spleen cell subset positive separation of T cells (mouse CD38 microbeads), B cells (CD19 microbeads), monocytes/macrophages (CD11b microbeads), NK cells (CD49b microbeads) and DCs (CD11c microbeads).

Culture Conditions of Linker Sequences with Spleen Cells and Prostate Tumor Cells for Assay Performance

1. After exposure to the linker oligonucleotide TLR 2, 4, 7, 8 and 9, expression is evaluated using flow cytometry and compared to cells not exposed to linker sequence.

2. Commercially available antibodies to mouse TLRs include: (clone mT2.7-TLR2, clone UT41-TLR4, LS-C148755-TLR7 (polyclonal LifeSpan BioSciences), 44C143-TLR8, M9.D6-TLR9.

3. 1×10⁶ cells/well are cultured in 96-well plates with 200 μl of media and oligonucleotide from 0.002 μg, 0.02 μg and 0.2 μg for 6 hrs and 24 hrs in triplicate. The oligonucleotide concentration is expanded if necessary to examine the entire dynamic range of the impact of the indicated oligonucleotides upon mouse spleen cell subsets.

Immunostaining for TLR 2, 4, 7, 8 and 9 by Flow Cytometry on TRAMP-C1, Syngeneic T and B Cell Lines and Splenic T and B Cells

1. Harvest then wash the cells in PBS, 10% FCS, 0.1% sodium azide and adjust cell suspension to a concentration of 1-5×10⁶ cells/ml in ice cold PBS, 10% FCS, 1% sodium azide in polystyrene round bottom 12×75 mm² Falcon tubes. Staining of the indicated cells for flow cytometry uses ice cold reagents/solutions and at 4° C., as low temperature and presence of sodium azide prevent the modulation and internalization of surface antigens.

2. Add 0.1-10 μg/ml of the primary fluorescently labeled anti-TLR7, anti-TLR8 or anti-TLR9 antibodies. Dilutions, if necessary, are made in 3% BSA/PBS (Propidium iodide can also be added at this point for dead cell exclusion). The Ab concentration and staining times/conditions may need to be optimized for each Ab and/or cell type.

3. Incubate for at least 30 min at room temperature or 4° C.

4. Wash the cells 3× by centrifugation at 400 g for 5 minutes and resuspend in 500 μl to 1 ml of ice cold PBS, 10% FCS and 1% sodium azide. Keep the cells in the dark on ice or at 4° C. in a fridge until the scheduled time for analysis.

Evaluate Effects of Immunomodulatory Oligos Upon Relevant Cells

The effect of promising immunomodulatory linker sequences as determined above is assessed using desired cell lines, e.g., mouse immature DC/monocyte cell lines, NK cells and T cells and B cell lines. The assayed immune cells may be chosen to be syngeneic to the tumor cells (e.g., C57B1-6 background). Cells are assessed for function, maturation and phenotype. Assays include cytokine secretion, co-activator molecules and maturation markers on DCs; perforin expression and activation/maturation markers on T/B cells, as further detail below:

Phenotype DC/Monocyte Cells, NK Cells, T Cells and B Cells by Flow Cytometry:

Cell activation is assessed using flow cytometry with the fluorescent antibody staining procedure above. B cell activation is assessed using fluorescent anti-CD86, -CD70, -CD40, and MHC class I and II antibodies. Assessment of T cell activation uses anti-CD28, and anti-CD137 antibodies. Assessment of NK activation uses anti-CD69 and anti-161 antibodies. Macrophage activation is evaluated using anti-CD63, anti-CD64 and anti-CD163.

Cytokine Assays:

Cytokine assays for anti- and pro-inflammatory cytokines use the BD Cytometric Bead Array (CBA) (BD Biosciences, San Jose, Calif., USA) with any desired confirmation of results using conventional ELISA or multiplex flow bead based assays (e.g., systems available from Luminex Corp, Austin Tex.) both with integrated standard curves for quantification. An automated screening process of individual cell lines with simultaneous evaluation of secreted cytokines (ELISA/flow bead cytokine assays), proliferation/apoptosis (Vibrant® MTT Cell Proliferation Assay Kit, Cat #V13154, according to the manufacturer's published protocols (Life Technologies, Carlsbad, Calif. USA)) and maturation/functional phenotyping (flow cytometry). Cell types expected to be impacted by the trivalent aptamer are evaluated including immune and target cancer cells.

Maturation and Function Marker Phenotyping:

Maturation and function marker phenotyping are performed using flow cytometry using commercially available antibodies as described herein.

The immunoregulatory linkers can be designed to induce apoptosis. Apoptosis in the prostate cancer and immune cells are evaluated, as further detailed below:

Apoptosis-Necrosis Studies:

Apoptosis/necrosis in immune cell lines and prostate cancer cells is determined using standard apoptosis assays known in the art. These include propidium iodide (PI) vs. Annexin V staining and the presence of hypodiploid peak in PI labeled cells detected using conventional flow cytometry.

TLR activation of cancer cells may be an unintended consequence of the linker. This is assessed on the target mouse prostate cancer cell lines (e.g., TRAMP-C1 mouse prostate cell line) using proliferation, motility and invasion assays, as further detailed below:

Motility Assays:

Motility assays use Transwell Migration Assay (Life Technologies) with target cells placed in the upper chamber and chemoattractant media in the lower chamber with the migrating cells quatified. Briefly, cells are serum starved, harvested, counted and placed into the upper chamber of the Transwell system. A suitable chemoattractant for each cell type is placed in to the lower chamber and the cells are allowed to traverse the porous membrane for 12-14 hours (depending on inherent motility of each cell type). The percentage of cells that traverse the membrane are counted to provide the motility index (number of cells on lower membrane face/total number of cells added to upper chamber times 100).

Invasion Assays:

For invasion assay a matrigel “barrier” is placed above the mesh so that the cells must digest this artificial extracellular matrix (ECM) to escape which is biologic proxy for invading healthy tissue. Such methods are known in the art. See, e.g., BD Matrigel™ matrix from BD Biosciences (San Jose, Calif.).

### Example 53

### Tripartite Aptamer Assessment

The linker and binding regions described above (see Examples 51-52) are assembled into a tripartite aptamer as shown in FIG. 33A. Based on the results of these studies above, the optimal oligonucleotide segments are synthesized in-line and evaluated as a unit in the same cell subsets/types and same assays as the individual segments were evaluated. See Examples 51-52. The oligonucleotide is synthesized with a biotin tag so that conventional streptavidin-phycoerythrin (SA-PE) assays will confirm binding to TRAMP-C1 associated microvesicles.

The trivalent aptamer is assessed to confirm binding to target cMVs and cells and to confirm that such binding of the trivalent aptamer induces the desired effects on immune cells, as outlined above. Binding of the aptamer to target cMVs and cells is also confirmed in a co-culture in vitro model composed of mouse spleen cells and TRAMP-C1 mouse prostate cell line or the like. Studies are carried out as detailed below:

Binding Studies:

The aptamers incorporate biotin molecules to facilitate strepatavidin-phycoerythrin (SA-PE) labeling in order to visualize binding to relevant immune cell subsets and to tumor cells. Cell binding is observed with fluorescent microscopy or flow cytometry. Luminex bead assays are used to confirm binding on TRAMP-C1-derived microvesicles.

Flow cytometry is also used to confirm that TRAMP-C1 microvesicles also bind the trivalent aptamer structure. Microvesicles are detected using fluorescently labeled anti-tetraspanin antibodies (e.g., anti-CD9, anti-CD63, anti-CD81) or other general vesicle markers. Microvesicles bound by the trivalent aptamer stain positive for the anti-tetraspanin and aptamer labels.

In Vitro Model:

The splenic immune cells will be derived from hyaluronic acid, collagenase and DNase-digested syngeneic mouse spleens which are notable for increased residual splenic DCs and macrophages which are not typically recovered by conventional spleen cell isolation techniques:

a. Disruption and digestion of mouse spleens for immune cells. See Ciavarra et al., 2000.

b. Miltenyi magnetic bead separation of spleen cell subtypes (Miltenyi Biotec's MACS System is used according to manufacturer's protocols for mouse spleen cell subset positive separation of T cells; (mouse CD3∈ microbeads), B cells (CD19 microbeads), monocytes/macrophages (CD11b microbeads), NK cells (CD49b microbeads) and DCs (CD11c microbeads).

c. Culture conditions of trivalent aptamer structure with spleen cells and prostate tumor cells at 5×10⁶ cells/well, 12-24 hours incubation, with the same assays described above for individual aptamer components.

For the in vitro model, a 10:1 ratio of splenic cells to mouse prostate cancer cells in complete RMPI media is used. Initial optimization studies employ a matrix analysis with control media and various concentration of the aptamer molecule and assessment of cell culture characteristics at various time points including 0 hrs, 3 hrs, 6 hrs, 24 hrs, 48 hrs and 72 hrs post-addition of the aptamer with varying concentrations of the trivalent aptamer. Prostate tumor cells are labeled with a non-toxic “cell tracker” dye to facilitate the quantification of the tumor cells. Binding of the trivalent oligonucleotide is determined by multiparametric flow cytometry including cell tracker labeling of TRAMP-C1 cells, fluorescent antibodies for immune cells and SA-PE labeling of aptamer complex to confirm binding.

Once appropriate culture conditions are determined, experiments that assess the effects of the trivalent aptamer on the immunosuppressive environment of tumor-derive cMV and tumor cells are performed. Harvested cultures will be analyzed with multiparametric flow cytometry assays including but not limited to cell sub-type identifier markers, activation and maturation markers, cytokine secretion by cell type with Golgi blocked cells and cell counts. Prostate tumor cells are labeled with a non-toxic “cell tracker” dye to facilitate the quantification of the tumor cells with the aptamers.

Optimization:

The sequence of the aptamer, including both binding regions and the linker region can be modified and assessed to further optimize the sequence.

Pre-Clinical Studies:

Animal models are used to assess aptamer treatment vs. tumor growth and survival studies. In vivo studies in mice are performed to demonstrate that the trivalent aptamers slow the growth of neoplastic cells and/or prolong survival in mice with ectopic TRAMP-C1 (or equivalent) tumors. Various doses of aptamers are assessed to determine the optimal dose vs. carrier vehicle i.p. daily. Because TRAMP-C1 tumors are fairly slow growing when injected ectopically, 5×10⁵ cells are implanted subcutaneously and therapy is started after three days. Tumors are measured in two dimensions (mm²) three times per week during the course of the experiment. Published reports indicate these tumors are expected to be lethal within 60 days if not treated. Growth kinetics of the tumors are monitored. The endpoint of these studies includes survival until the tumors become too large to be humanely born by the mice according to guidelines. Groups of 20 controls and 20 treated mice are used to demonstrate differences in tumor growth kinetics and survival with treatment.

Determine In Vivo Efficacy of Trivalent Aptamer.

Forty C57B1-6 male mice are injected subcutaneously with 5×10⁶ syngeneic TRAMP-C1 cells. Aptamer therapy is initiated three days after the injection. Treatment consists of 60 daily consecutive i.p. injections of either carrier (20 inoculated mice to receive 0.1% normal mouse serum in PBS) or therapeutic agent (20 mice to receive the trivalent aptamer in PBS solution). Tumor volumes are obtained two times a week by the measurement of bisecting tumor diameters (mm²) during the treatment period. Mice whose tumors exceed 10% of body weight or who become moribund because of metastasis of the TRAMP tumors are humanely sacrificed. A significant response to the trivalent aptamer therapy is defined as the reduction of the tumor volume using bisecting tumor diameters greater than 2×S.D using one-tailed Student's t-test or when ANOVA analysis provides a p>0.05.

At the completion of the therapeutic period (days+3 through +63) surviving mice are monitored and plotted for survival using Kaplan-Meier plots as standard in the art.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of detecting a presence or level of one or more microvesicle in a biological sample, comprising:
(a) contacting a biological sample with a lipid staining dye, wherein the biological sample comprises or is suspected to comprise the one or more microvesicle; and
(b) detecting the lipid staining dye in contact with the one or more microvesicle, thereby detecting the presence or level of the one or more microvesicle.

2. The method of claim 1, wherein the lipid staining dye comprises a long-chain dialkylcarbocyanine, an indocarbocyanine (DiI), an oxacarbocyanine (DiO), FM 1-43, FM 1-43FX, FM 4-64, FM 5-95, a dialkyl aminostyryl dye, DiA, a long-wavelength light-excitable carbocyanines (DiD), an infrared light-excitable carbocyanine (DiR), carboxyfluorescein succinimidyl ester (CFDA), carboxyfluorescein succinimidyl ester (CFSE), 4-(4-(Dihexadecylamino)styryl)-N-Methylpyridinium Iodide (DiA; 4-Di-16-ASP), 4-(4-(Didecylamino)styryl)-N-Methylpyridinium Iodide (4-Di-10-ASP), 1,1′-Dioctadecyl-3,3,3′,3′-Tetramethylindodicarbocyanine Perchlorate (‘DiD’ oil; DiIC₁₈(5) oil), 1,1′-Dioctadecyl-3,3,3′,3′-Tetramethylindodicarbocyanine, 4-Chlorobenzenesulfonate Salt (‘DiD’ solid; DiIC₁₈(5) solid), 1,1′-Dioleyl-3,3,3′,3′-Tetramethylindocarbocyanine methanesulfonate (Δ⁹-DiI), Dil Stain (1,1′-Dioctadecyl-3,3,3′,3′-Tetramethylindocarbocyanine Perchlorate (‘DiI’; DiIC₁₈(3))), Dil Stain (1,1′-Dioctadecyl-3,3,3′,3′-Tetramethylindocarbocyanine Perchlorate (‘DiI’; DiIC₁₈(3))), 1,1′-Didodecyl-3,3,3′,3′-Tetramethylindocarbocyanine Perchlorate (DiIC₁₂(3)), 1,1′-Dihexadecyl-3,3,3′,3′-Tetramethylindocarbocyanine Perchlorate (DiIC₁₆(3)), 1,1′-Dioctadecyl-3,3,3′,3′-Tetramethylindocarbocyanine-5,5′-Disulfonic Acid (DiIC₁₈(3)-DS), 1,1′-Dioctadecyl-3,3,3′,3′-Tetramethylindodicarbocyanine-5,5′-Disulfonic Acid (DiIC₁₈(5)-DS), 4-(4-(Dilinoleylamino)styryl)-N-Methylpyridinium 4-Chlorobenzenesulfonate (FAST DiA™ solid; DiΔ^{9,12}-C_{B}ASP, CBS), 3,3′-Dilinoleyloxacarbocyanine Perchlorate (FAST DiO™ Solid; DiOA^{9,12}-C₁₈(3), ClO₄), 1,1′-Dilinoleyl-3,3,3′,3′-Tetramethylindocarbocyanine, 4-Chlorobenzenesulfonate (FAST DiI™ solid; DiIA^{9,12}-C₁₈(3), CBS), 1,1′-Dilinoleyl-3,3,3′,3′-Tetramethylindocarbocyanine Perchlorate (FAST DiI™ oil; DiIA^{9,12}-C₁₈(3), ClO₄), 3,3′-Dioctadecyloxacarbocyanine Perchlorate (‘DiO’; DiOC₁₈(3)), 3,3′-Dihexadecyloxacarbocyanine Perchlorate (DiOC₁₆(3)), 3,3′-Dioctadecyl-5,5′-Di(4-Sulfophenyl)Oxacarbocyanine, Sodium Salt (SP-DiOC₁₈(3)), 1,1′-Dioctadecyl-6,6′-Di(4-Sulfophenyl)-3,3,3′,3′-Tetramethylindocarbocyanine (SP-DiIC₁₈(3)), 1,1′-Dioctadecyl-3,3,3′,3′-Tetramethylindotricarbocyanine Iodide (DiR; DiIC₁₈(7)), 3,3′-Diethylthiacarbocyanine iodide, 3,3′-Diheptylthiacarbocyanine iodide, 3,3′-Dioctylthiacarbocyanine iodide, 3,3′-Dipropylthiadicarbocyanine iodide, 7-(Diethylamino)coumarin-3-carboxylic acid, 7-(Diethylamino)coumarin-3-carboxylic acid N-succinimidyl ester, an analog or variant of any thereof, and a combination of any thereof.

3. The method of claim 1, wherein the lipid staining dye is labeled.

4. The method of claim 1, wherein the lipid staining dye is converted from a non-labeled form to a labeled form upon contact with the microvesicle.

5. The method of claim 4, wherein the lipid staining dye comprises an esterase-activated lipophilic dye.

6. The method of claim 5, wherein the esterase-activated lipophilic dye comprises carboxyfluorescein succinimidyl ester (CFDA).

7. The method of claim 6, wherein the CFDA is converted into carboxyfluorescein succinimidyl ester (CFSE) upon contact with microvesicle esterases.

8. The method of any preceding claim, wherein the biological sample comprises a bodily fluid.

9. The method of claim 8, wherein the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, or a derivative of any thereof.

10. The method of any preceding claim, wherein the biological sample comprises peripheral blood, serum or plasma.

11. The method of any of claims 8-10, further comprising selectively depleting one or more abundant protein from the biological sample prior to step (a).

12. The method of any of claims 8-10, further comprising selectively depleting one or more abundant protein from the biological sample prior to step (b).

13. The method of any of claims 1-7, wherein the biological sample comprises a cell culture sample or a tissue sample.

14. The method of any preceding claim, further comprising detecting one or more microvesicle antigen associated with the one or more microvesicle.

15. The method of claim 14, wherein the one or more microvesicle-associated antigen is selected from Table 3, Table 4, and/or Table 5.

16. The method of claim 14, wherein the one or more microvesicle-associated antigen comprises a protein selected from the group consisting of ADAM 34, ADAM 9, AGR2, ALDOA, ANXA1, ANXA 11, ANXA4, ANXA 7, ANXA2, ARF6, ATP1A1, ATP1A2, ATP1A3, BCHE, BCL2L14 (Bcl G), BDKRB2, CA215, CAV1-Caveolinl, CCR2 (CC chemokine receptor 2, CD192), CCR5 (CC chemokine receptor 5), CCT2 (TCP1-beta), CD166/ALCAM, CD49b (Integrin alpha 2, ITGA4), CD90/THY1, CDH1, CDH2, CDKN1A cyclin-dependent kinase inhibitor (p21), CGA gene (coding for the alpha subunit of glycoprotein hormones), CHMP4B, CLDN3-Claudin3, CLSTN1 (Calsyntenin-1), COX2 (PTGS2), CSE1L (Cellular Apoptosis Susceptibility), Cytokeratin 18, Eag1 (KCNH1) (plasma membrane-K+-voltage gated channel), EDIL3 (del-1), EDNRB—Endothelial Receptor Type B, Endoglin/CD105, ENOX2-Ecto-NOX disulphide Thiol exchanger 2, EPCA-2 Early prostate cancer antigen2, EpoR, EZH2 (enhancer of Zeste Homolog2), EZR, FABP5, Farnesyltransferase/geranylgeranyl diphosphate synthase 1 (GGPS1), Fatty acid synthase (FASN, plasma membrane protein), FTL (light and heavy), GDF15-Growth Differentiation Factor 15, GloI, GSTP1, H3F3A, HGF (hepatocyte growth factor), hK2 (KLK2), HSP90AA1, HSPA1A/HSP70-1, IGFBP-2, IGFBP-3, IL1alpha, IL-6, IQGAP1, ITGAL (Integrin alpha L chain), Ki67, KLK1, KLK10, KLK11, KLK12, KLK13, KLK14, KLK15, KLK4, KLK5, KLK6, KLK7, KLK8, KLK9, Lamp-2, LDH-A, LGALS3BP, LGALS8, MFAP5, MMP 1, MMP 2, MMP 24, MMP 25, MMP 3, MMP10, MMP-14/MT1-MMP, MTA1, nAnS, Nav1.7, NCAM2-Neural cell Adhesion molecule 2, NGEP/D-TMPP/IPCA-5/ANO7, NKX3-1, Notch1, NRP1/CD304, PGP, PAP (ACPP), PCA3-Prostate cancer antigen 3, Pdia3/ERp57, PhIP, phosphatidylethanolamine (PE), PIP3, PKP1 (plakophilin1), PKP3 (plakophilin3), Plasma chromogranin-A (CgA), PRDX2, Prostate secretory protein (PSP94)/β-Microseminoprotein (MSP)/IGBF, PSAP, PSMA1, PTEN, PTGFRN, PTPN13/PTPL1, PKM2, RPL19, SCA-1/ATXN1, SERINC5/TPO1, SET, SLC3A2/CD98, STEAP1, STEAP-3, SRVN, Syndecan/CD138, TGFB, Tissue Polypeptide Specific antigen TPS, TLR4 (CD284), TLR9 (CD289), TMPRSS1/hepsin, TMPRSS2, TNFR1, TNFα, CD283/TLR3, Transferrin receptor/CD71/TRFR, uPA (urokinase plasminoge activator), uPAR (uPA receptor)/CD87, VEGFR1, VEGFR2, and a combination thereof.

17. The method of claim 14, wherein the one or more microvesicle-associated antigen comprises a protein selected from the group consisting of ADAM 9, ADAM10, AGR2, ALDOA, ALIX, ANXA1, ANXA2, ANXA4, ARF6, ATP1A3, B7H3, BCHE, BCL2L14 (Bcl G), BCNP1, BDKRB2, BDNFCAV1-Caveolinl, CCR2 (CC chemokine receptor 2, CD192), CCR5 (CC chemokine receptor 5), CCT2 (TCP1-beta), CD10, CD151, CD166/ALCAM, CD24, CD283/TLR3, CD41, CD46, CD49d (Integrin alpha 4, ITGA4), CD63, CD81, CD9, CD90/THY1, CDH1, CDH2, CDKN1A cyclin-dependent kinase inhibitor (p21), CGA gene (coding for the alpha subunit of glycoprotein hormones), CLDN3-Claudin3, COX2 (PTGS2), CSE1L (Cellular Apoptosis Susceptibility), CXCR3, Cytokeratin 18, Eag1 (KCNH1), EDIL3 (del-1), EDNRB-Endothelial Receptor Type B, EGFR, EpoR, EZH2 (enhancer of Zeste Homolog2), EZR, FABP5, Farnesyltransferase/geranylgeranyl diphosphate synthase 1 (GGPS1), Fatty acid synthase (FASN), FTL (light and heavy), GAL3, GDF15-Growth Differentiation Factor 15, GloI, GM-CSF, GSTP1, H3F3A, HGF (hepatocyte growth factor), hK2/Kif2a, HSP90AA1, HSPA1A/HSP70-1, HSPB1, IGFBP-2, IGFBP-3, IL1alpha, IL-6, IQGAP1, ITGAL (Integrin alpha L chain), Ki67, KLK1, KLK10, KLK11, KLK12, KLK13, KLK14, KLK15, KLK4, KLK5, KLK6, KLK7, KLK8, KLK9, Lamp-2, LDH-A, LGALS3BP, LGALS8, MMP 1, MMP 2, MMP 25, MMP 3, MMP10, MMP-14/MT1-MMP, MMP7, MTA1nAnS, Nav1.7, NKX3-1, Notch1, NRP1/CD304, PAP (ACPP), PGP, PhIP, PIP3/BPNT1, PKM2, PKP1 (plakophilin1), PKP3 (plakophilin3), Plasma chromogranin-A (CgA), PRDX2, Prostate secretory protein (PSP94)/β-Microseminoprotein (MSP)/IGBF, PSAP, PSMA, PSMA1, PTENPTPN13/PTPL1, RPL19, seprase/FAPSET, SLC3A2/CD98, SRVN, STEAP1, Syndecan/CD138, TGFB, TGM2, TIMP-1TLR4 (CD284), TLR9 (CD289), TMPRSS1/hepsin, TMPRSS2, TNFR1, TNFα, Transferrin receptor/CD71/TRFR, Trop2 (TACSTD2), TWEAK uPA (urokinase plasminoge activator) degrades extracellular matrix, uPAR (uPA receptor)/CD87, VEGFR1, VEGFR2, and a combination thereof.

18. The method of claim 14, wherein the one or more microvesicle-associated antigen comprises a protein selected from the group consisting of A33, ABL2, ADAM10, AFP, ALA, ALIX, ALPL, ApoJ/CLU, ASCA, ASPH(A-10), ASPH(D01P), AURKB, B7H3, B7H3, B7H4, BCNP, BDNF, CA125(MUC16), CA-19-9, C-Bir, CD10, CD151, CD24, CD41, CD44, CD46, CD59(MEM-43), CD63, CD63, CD66eCEA, CD81, CD81, CD9, CD9, CDA, CDADC1, CRMP-2, CRP, CXCL12, CXCR3, CYFRA21-1, DDX-1, DLL4, DLL4, EGFR, Epcam, EphA2, ErbB2, ERG, EZH2, FASL, FLNA, FRT, GAL3, GATA2, GM-CSF, Gro-alpha, HAP, HER3(ErbB3), HSP70, HSPB1, hVEGFR2, iC3b, IL-1B, IL6R, IL6Unc, IL7Ralpha/CD127, IL8, INSIG-2, Integrin, KLK2, LAMN, Mammoglobin, M-CSF, MFG-E8, MIF, MISRII, MMP7, MMP9, MUC1, Muc1, MUC17, MUC2, Ncam, NDUFB7, NGAL, NK-2R(C-21), NT5E (CD73), p53, PBP, PCSA, PCSA, PDGFRB, PIM1, PRL, PSA, PSA, PSMA, PSMA, RAGE, RANK, RegIV, RUNX2, S100-A4, seprase/FAP, SERPINB3, SIM2(C-15), SPARC, SPC, SPDEF, SPP1, STEAP, STEAP4, TFF3, TGM2, TIMP-1, TMEM211, Trail-R2, Trail-R4, TrKB(poly), Trop2, Tsg101, TWEAK, UNC93A, VEGFA, wnt-5a(C-16), and a combination thereof.

19. The method of claim 18, wherein the one or more microvesicle-associated antigen further comprises a protein selected from the group consisting of CD9, CD63, CD81, PCSA, MUC2, MFG-E8, and a combination thereof.

20. The method of claim 14, wherein the one or more microvesicle-associated antigen comprises 5HT2B, 5T4 (trophoblast), ACO2, ACSL3, ACTN4, ADAM10, AGR2, AGR3, ALCAM, ALDH6A1, ANGPTL4, ANO9, AP1G1, APC, APEX1, APLP2, APP (Amyloid precursor protein), ARCN1, ARHGAP35, ARL3, ASAH1, ASPH (A-10), ATP1B1, ATP1B3, ATP5I, ATP5O, ATXN1, B7H3, BACE1, BAI3, BAIAP2, BCA-200, BDNF, BigH3, BIRC2, BLVRB, BRCA, BST2, C1GALT1, C1GALT1C1, C20orf3, CA125, CACYBP, Calmodulin, CAPN1, CAPNS1, CCDC64B, CCL2 (MCP-1), CCT3, CD10(BD), CD127 (IL7R), CD174, CD24, CD44, CD80, CD86, CDH1, CDH5, CEA, CFL2, CHCHD3, CHMP3, CHRDL2, CIB1, CKAP4, COPA, COX5B, CRABP2, CRIP1, CRISPLD1, CRMP-2, CRTAP, CTLA4, CUL3, CXCR3, CXCR4, CXCR6, CYB5B, CYB5R1, CYCS, CYFRA 21, DBI, DDX23, DDX39B, derlin 1, DHCR7, DHX9, DLD, DLL4, DNAJBL DPP6, DSTN, eCadherin, EEF1D, EEF2, EFTUD2, EIF4A2, EIF4A3, EpCaM, EphA2, ER(1) (ESR1), ER(2) (ESR2), Erb B4, Erb2, erb3 (Erb-B3), ERLIN2, ESD, FARSA, FASN, FEN1, FKBP5, FLNB, FOXP3, FUS, Gal3, GCDPF-15, GCNT2, GNAl2, GNG5, GNPTG, GPC6, GPD2, GPER (GPR30), GSPT1, H3F3B, H3F3C, HADH, HAP1, HER3, HIST1H1C, HIST1H2AB, HIST1H3A, HIST1H3C, HIST1H3D, HIST1H3E, HIST1H3F, HIST1H3G, HIST1H3H, HIST1H3I, HIST1H3J, HIST2H2BF, HIST2H3A, HIST2H3C, HIST2H3D, HIST3H3, HMGB1, HNRNPA2B1, HNRNPAB, HNRNPC, HNRNPD, HNRNPH2, HNRNPK, HNRNPL, HNRNPM, HNRNPU, HPS3, HSP-27, HSP70, HSP90B1, HSPA1A, HSPA2, HSPA9, HSPE1, IC3b, IDE, IDH3B, IDO1, IFI30, IL1RL2, IL7, IL8, ILF2, ILF3, IQCG, ISOC2, IST1, ITGA7, ITGB7, junction plakoglobin, Keratin 15, KRAS, KRT19, KRT2, KRT7, KRT8, KRT9, KTN1, LAMP1, LMNA, LMNB1, LNPEP, LRPPRC, LRRC57, Mammaglobin, MAN1A1, MAN1A2, MART″, MATR3, MBD5, MCT2, MDH2, MFGE8, MFGE8, MGP, MMP9, MRP8, MUC1, MUC17, MUC2, MYO5B, MYOF, NAPA, NCAM, NCL, NG2 (CSPG4), Ngal, NHE-3, NME2, NONO, NPM1, NQO1, NT5E (CD73), ODC1, OPG, OPN (SC), 0S9, p53, PACSIN3, PAICS, PARK7, PARVA, PC, PCNA, PCSA, PD-1, PD-L1, PD-L2, PGP9.5, PHB, PHB2, PIK3C2B, PKP3, PPL, PR(B), PRDX2, PRKCB, PRKCD, PRKDC, PSA, PSAP, PSMA, PSMB7, PSMD2, PSME3, PYCARD, RAB1A, RAB3D, RAB7A, RAGE, RBL2, RNPEP, RPL14, RPL27, RPL36, RPS25, RPS4X, RPS4Y1, RPS4Y2, RUVBL2, SET, SHMT2, SLAIN″, SLC39A14, SLC9A3R2, SMARCA4, SNRPD2, SNRPD3, SNX33, SNX9, SPEN, SPR, SQSTM1, SSBP1, ST3GAL1, STXBP4, SUB1, SUCLG2, Survivin, SYT9, TFF3 (secreted), TGOLN2, THBS1, TIMP1, TIMP2, TMED10, TMED4, TMED9, TMEM211, TOM1, TRAF4 (scaffolding), TRAIL-R2, TRAP1, TrkB, Tsg 101, TXNDC16, U2AF2, UEVLD, UFC1, UNC93a, USP14, VASP, VCP, VDAC1, VEGFA, VEGFR1, VEGFR2, VPS37C, WIZ, XRCC5, XRCC6, YB-1, YWHAZ, or any combination thereof.

21. The method of any preceding claim, wherein the one or more binding agent comprises a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof.

22. The method of any preceding claim, wherein the one or more binding agent comprises an antibody and/or an aptamer.

23. The method of any preceding claim, wherein the one or more microvesicle is subjected to size exclusion chromatography, density gradient centrifugation, differential centrifugation, nanomembrane ultrafiltration, immunoabsorbent capture, affinity purification, affinity capture, immunoassay, microfluidic separation, flow cytometry or combinations thereof.

24. The method of any preceding claim, further comprising detecting one or more payload biomarker within the one or more microvesicle.

25. The method of claim 24, wherein the one or more payload biomarker comprises one or more nucleic acid, peptide, protein, lipid, antigen, carbohydrate, and/or proteoglycan.

26. The method of claim 25, wherein the nucleic acid comprises one or more DNA, mRNA, microRNA, snoRNA, snRNA, rRNA, tRNA, siRNA, hnRNA, or shRNA.

27. The method of claim 24, wherein the one or more payload biomarker comprises mRNA.

28. The method of any preceding claim, wherein the detected presence or level the one or more microvesicle is used to characterize a cancer.

29. The method of claim 28, wherein the concentration of the detected microvesicles is compared to a reference in order to characterize the cancer.

30. The method of claim 28, wherein the characterizing comprises providing a prognostic, diagnostic or theranostic determination for the cancer, identifying the presence or risk of the cancer, or identifying the cancer as metastatic or aggressive.

31. The method of any of claims 28-30, where the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma;
pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

32. The method of claim 31, wherein the cancer comprises prostate cancer.

33. The method of claim 31, wherein the cancer comprises breast cancer.

34. The method of any preceding claim, wherein the method is performed in vitro.

35. Use of the one or more reagent to carry out the method of any preceding claim.

36. Use of a reagent for the manufacture of a kit or reagent for carrying out the method of any of claims 1-34.

37. A kit comprising one or more reagent to carry out the method of any of claims 1-34.

38. The use of any of claims 35-36 or the kit of claim 37, wherein the one or more reagent is selected from the group consisting of one or more reagent capable of binding to a microvesicle surface antigen, a filtration unit, a dilution buffer, an affinity column to remove one or more abundant protein, one or more lipophilic dye, one or more population of microvesicles, and a combination thereof.

39. An aptamer that comprises a first binding region to a first target, a second binding region to a second target, and a linker region between the first binding region and the second binding region.

40. The aptamer of claim 39, wherein the first target comprises a cancer or cell-of-origin specific protein marker.

41. The aptamer of claim 39, wherein the first target comprises a microvesicle surface antigen.

42. The aptamer of claim 39, wherein the first target is selected from any of Table 3, Table 4 or Table 5.

43. The aptamer of claim 39, wherein the first target is selected from the group consisting of 5T4, A33, ACTG1, ADAM10, ADAM15, AFP, ALA, ALDOA, ALIX, ALP, ALX4, ANCA, Annexin V, ANXA2, ANXA6, APC, APOA1, ASCA, ASPH, ATP1A1, AURKA, AURKB, B7H3, B7H4, BANK1, BASP1, BCA-225, BCNP1, BDNF, BRCA, C1orf58, C20orf114, C8B, CA125 (MUC16), CA-19-9, CAPZA1, CAV1, C-Bir, CCSA-2, CCSA-3&4, CD1.1, CD10, CD151, CD174 (Lewis y), CD24, CD2AP, CD37, CD44, CD46, CD53, CD59, CD63, CD66 CEA, CD73, CD81, CD82, CD9, CDA, CDAC1 1a2, CEA, C-Erbb2, CFL1, CFP, CHMP4B, CLTC, COTL1, CRMP-2, CRP, CRTN, CTNND1, CTSB, CTSZ, CXCL12, CYCS, CYFRA21-1, DcR3, DLL4, DPP4, DR3, EEF1A1, EGFR, EHD1, ENO1, EpCAM, EphA2, ER, ErbB4, EZH2, F11R, F2, F5, FAM125A, FASL, Ferritin, FNBP1L, FOLH1, FRT, GAL3, GAPDH, GDF15, GLB1, GPCR (GPR110), GPR30, GPX3, GRO-1, Gro-alpha, HAP, HBD 1, HBD2, HER 3 (ErbB3), HIST1H1C, HIST1H2AB, HNP1-3, HSP, HSP70, HSP90AB1, HSPA1B, HSPA8, hVEGFR2, iC3b, ICAM, IGSF8, IL 6, IL-1B, IL6R, IL8, IMP3, INSIG-2, ITGB1, ITIH3, JUP, KLK2, L1CAM, LAMN, LDH, LDHA, LDHB, LUM, LYZ, MACC-1, MAPK4, MART-1, MCP-1, M-CSF, MFGE8, MGAM, MGC20553, MIC1, MIF, MIS RII, MMG, MMP26, MMP7, MMP9, MS4A1, MUC1, MUC17, MUC2, MYH2, MYL6B, Ncam, NGAL, NME1, NME2, NNMT, NPGP/NPFF2, OPG, OPG-13, OPN, p53, PA2G4, PABPC1, PABPC4, PACSIN2, PBP, PCBP2, PCSA, PDCD6IP, PDGFRB, PGP9.5, PIM1, PR (B), PRDX2, PRL, PSA, PSCA, PSMA, PSMA1, PSMA2, PSMA4, PSMA6, PSMA7, PSMB1, PSMB2, PSMB3, PSMB4, PSMB5, PSMB6, PSMB8, PSME3, PTEN, PTGFRN, Rab-5b, Reg IV, RPS27A, RUNX2, SCRN1, SDCBP, seprase, Sept-9, SERINC5, SERPINB3, SERPINB3, SH3GL1, SLC3A2, SMPDL3B, SNX9, SPARC, SPB, SPDEF, SPON2, SPR, SRVN, SSX2, SSX4, STAT 3, STEAP, STEAP1, TACSTD1, TCN2, tetraspanin, TF (FL-295), TFF3, TGM2, THBS1, TIMP, TIMP1, TIMP2, TMEM211, TMPRSS2, TNF-alpha, TPA, TPI1, TPS, Trail-R2, Trail-R4, TrKB, TROP2, TROP2, Tsg 101, TUBB, TWEAK, UNC93A, VDAC2, VEGF A, VPS37B, YPSMA-1, YWHAG, YWHAQ, and YWHAZ.

44. The aptamer of claim 39, wherein the first target is selected from the group consisting of 5HT2B, 5T4 (trophoblast), ACO2, ACSL3, ACTN4, ADAM10, AGR2, AGR3, ALCAM, ALDH6A1, ANGPTL4, ANO9, AP1G1, APC, APEX1, APLP2, APP (Amyloid precursor protein), ARCN1, ARHGAP35, ARL3, ASAH1, ASPH (A-10), ATP1B1, ATP1B3, ATP5I, ATP5O, ATXN1, B7H3, BACE1, BAI3, BAIAP2, BCA-200, BDNF, BigH3, BIRC2, BLVRB, BRCA, BST2, C1GALT1, C1GALT1C1, C20orf3, CA125, CACYBP, Calmodulin, CAPN1, CAPNS1, CCDC64B, CCL2 (MCP-1), CCT3, CD10(BD), CD127 (IL7R), CD174, CD24, CD44, CD80, CD86, CDH1, CDH5, CEA, CFL2, CHCHD3, CHMP3, CHRDL2, CIB1, CKAP4, COPA, COX5B, CRABP2, CRIP1, CRISPLD1, CRMP-2, CRTAP, CTLA4, CUL3, CXCR3, CXCR4, CXCR6, CYB5B, CYB5R1, CYCS, CYFRA 21, DBI, DDX23, DDX39B, derlin 1, DHCR7, DHX9, DLD, DLL4, DNAJB1, DPP6, DSTN, eCadherin, EEF1D, EEF2, EFTUD2, EIF4A2, EIF4A3, EpCaM, EphA2, ER(1) (ESR1), ER(2) (ESR2), Erb B4, Erb2, erb3 (Erb-B3?), ERLIN2, ESD, FARSA, FASN, FEN1, FKBP5, FLNB, FOXP3, FUS, Gal3, GCDPF-15, GCNT2, GNAl2, GNG5, GNPTG, GPC6, GPD2, GPER (GPR30), GSPT1, H3F3B, H3F3C, HADH, HAP1, HER3, HIST1H1C, HIST1H2AB, HIST1H3A, HIST1H3C, HIST1H3D, HIST1H3E, HIST1H3F, HIST1H3G, HIST1H3H, HIST1H3I, HIST1H3J, HIST2H2BF, HIST2H3A, HIST2H3C, HIST2H3D, HIST3H3, HMGB1, HNRNPA2B1, HNRNPAB, HNRNPC, HNRNPD, HNRNPH2, HNRNPK, HNRNPL, HNRNPM, HNRNPU, HPS3, HSP-27, HSP70, HSP90B1, HSPA1A, HSPA2, HSPA9, HSPE1, IC3b, IDE, IDH3B, IDO1, IEI30, IL1RL2, IL7, IL8, ILF2, ILF3, IQCG, ISOC2, IST1, ITGA7, ITGB7, junction plakoglobin, Keratin 15, KRAS, KRT19, KRT2, KRT7, KRT8, KRT9, KTN1, LAMP1, LMNA, LMNB1, LNPEP, LRPPRC, LRRC57, Mammaglobin, MAN1A1, MAN1A2, MART1, MATR3, MBD5, MCT2, MDH2, MFGE8, MFGE8, MGP, MMP9, MRP8, MUC1, MUC17, MUC2, MYO5B, MYOF, NAPA, NCAM, NCL, NG2 (CSPG4), Ngal, NHE-3, NME2, NONO, NPM1, NQO1, NT5E (CD73), ODC1, OPG, OPN (SC), 0S9, p53, PACSIN3, PAICS, PARK7, PARVA, PC, PCNA, PCSA, PD-1, PD-L1, PD-L2, PGP9.5, PHB, PHB2, PIK3C2B, PKP3, PPL, PR(B), PRDX2, PRKCB, PRKCD, PRKDC, PSA, PSAP, PSMA, PSMB7, PSMD2, PSME3, PYCARD, RAB1A, RAB3D, RAB7A, RAGE, RBL2, RNPEP, RPL14, RPL27, RPL36, RPS25, RPS4X, RPS4Y1, RPS4Y2, RUVBL2, SET, SHMT2, SLAIN′, SLC39A14, SLC9A3R2, SMARCA4, SNRPD2, SNRPD3, SNX33, SNX9, SPEN, SPR, SQSTM1, SSBP1, ST3GAL1, STXBP4, SUB1, SUCLG2, Survivin, SYT9, TFF3 (secreted), TGOLN2, THBS1, TIMP1, TIMP2, TMED10, TMED4, TMED9, TMEM211, TOM1, TRAF4 (scaffolding), TRAIL-R2, TRAP1, TrkB, Tsg 101, TXNDC16, U2AF2, UEVLD, UFC1, UNC93a, USP14, VASP, VCP, VDAC1, VEGFA, VEGFR1, VEGFR2, VPS37C, WIZ, XRCC5, XRCC6, YB-1, YWHAZ, or any combination thereof.

45. The aptamer of claim 39, wherein the first target is selected from the group consisting of p53, p63, p73, mdm-2, procathepsin-D, B23, C23, PLAP, CA125, MUC-1, HER2, NY-ESO-1, SCP1, SSX-1, SSX-2, SSX-4, HSP27, HSP60, HSP90, GRP78, TAG72, HoxA7, HoxB7, EpCAM, ras, mesothelin, survivin, EGFK, MUC-1, or c-myc.

46. The aptamer of claim 39, wherein the second target comprises an immunosuppressive protein.

47. The aptamer of claim 39, wherein the second target is selected from the group consisting of TGF-β, CD39, CD73, IL10, FasL or TRAIL.

48. The aptamer of claim 39, wherein the second target is selected from the group consisting of FasL, programmed cell death 1 (PD-1), programmed death ligand-1 (PD-L1; B7-H1), programmed death ligand-2 (PD-L2; B7-DC), B7-H3, and B7-H4.

49. The aptamer of claim 39, wherein the linker region comprises an immune-modulatory oligonucleotide sequence.

50. The aptamer of claim 49, wherein the linker region comprises an immunostimulatory sequence.

51. The aptamer of claim 49, wherein the linker region comprises one or more CpG motif.

52. The aptamer of claim 49, wherein the linker region comprises a CpG region that is at least 50, 55, 60, 65, 70, 75, 80, 85, 90, 95, 96, 97, 98, 99 or 100 percent homologous to one or more of SEQ ID NOs. 2-4, or a functional fragment thereof.

53. The aptamer of claim 49, wherein the linker region comprises an anti-proliferative or pro-apoptotic sequence.

54. The aptamer of claim 49, wherein the linker region comprises a polyG sequence.

55. The aptamer of claim 49, wherein the linker region comprises a polyG region that is at least 50, 55, 60, 65, 70, 75, 80, 85, 90, 95, 96, 97, 98, 99 or 100 percent homologous to one or more of SEQ ID NOs. 5-10, or a functional fragment thereof.

56. The aptamer of claim 49, wherein the linker region comprises an immunostimulatory and an anti-proliferative or pro-apoptotic sequence.

57. The aptamer of claim 49, wherein the linker region comprises a hybrid CpG-polyG region that is at least 50, 55, 60, 65, 70, 75, 80, 85, 90, 95, 96, 97, 98, 99 or 100 percent homologous to one or more of SEQ ID NOs. 11-28, or a functional fragment thereof.

58. The aptamer of claim 39, wherein the aptamer is further modified to comprise at least one chemical modification.

59. The aptamer of claim 58, wherein the modification is selected from the group consisting: of a chemical substitution at a sugar position; a chemical substitution at a phosphate position; and a chemical substitution at a base position of the nucleic acid.

60. The aptamer of claim 58, wherein the modification is selected from the group consisting of:
incorporation of a modified nucleotide, 3′ capping, conjugation to an amine linker, conjugation to a high molecular weight, non-immunogenic compound, conjugation to a lipophilic compound, conjugation to a drug, conjugation to a cytotoxic moiety and labeling with a radioisotope.

61. The aptamer of claim 60, wherein the non-immunogenic, high molecular weight compound is polyalkylene glycol.

62. The aptamer of claim 61, wherein the polyalkylene glycol is polyethylene glycol.

63. The aptamer of claim 39, wherein the aptamer comprises an immunostimulating moiety.

64. The aptamer of claim 39, wherein the aptamer comprises a membrane disruptive moiety.

65. A pharmaceutical composition comprising a therapeutically effective amount of the aptamer of any of claims 39-64, or a salt thereof, and a pharmaceutically acceptable carrier or diluent.

66. A method of treating or ameliorating a disease associated with a neoplastic growth, comprising administering the composition of claim 65 to a patient in need thereof.

67. A kit comprising an aptamer of any of claims 39-64, or a pharmaceutical composition of claim 65.

68. A kit comprising a reagent for carrying out the method of claim 66.

69. Use of a reagent for carrying out the method of claim 66.

70. Use of a reagent for the manufacture of a kit or reagent for carrying out the method of claim 66.

71. Use of a reagent for the manufacture of a medicament for carrying out the method of claim 66.

72. The kit of claim 68 or use of any of claims 69-71, wherein the reagent comprises an aptamer of any of claims 39-64, or a pharmaceutical composition of claim 65.


SEQUENCE LISTING (28 sequences)

SEQ ID NO: 1 - DNA, 32 bp, Artificial Sequence
  source 1..32: /organism=“Artificial Sequence” /note=“Description of Artificial Sequence Synthetic oligonucleotide” /mol_type=“unassigned DNA”
ccccccgaatcacatgacttgggcgggggtcg

SEQ ID NO: 2 - DNA, 19 bp, Artificial Sequence
  source 1..19: /organism=“Artificial Sequence” /note=“Description of Artificial Sequence Synthetic oligonucleotide” /mol_type=“unassigned DNA”
tccatgacgttcctgatct

SEQ ID NO: 3 - DNA, 15 bp, Artificial Sequence
  source 1..15: /organism=“Artificial Sequence” /note=“Description of Artificial Sequence Synthetic oligonucleotide” /mol_type=“unassigned DNA”
gctagacgttagcgt

SEQ ID NO: 4 - DNA, 20 bp, Artificial Sequence
  source 1..20: /organism=“Artificial Sequence” /note=“Description of Artificial Sequence Synthetic oligonucleotide” /mol_type=“unassigned DNA”
atcgactctcgagcgttctc

SEQ ID NO: 5 - DNA, 15 bp, Artificial Sequence
  source 1..15: /organism=“Artificial Sequence” /note=“Description of Artificial Sequence Synthetic oligonucleotide” /mol_type=“unassigned DNA”
ggttggtgtggttgg

SEQ ID NO: 6 - DNA, 28 bp, Artificial Sequence
  source 1..28: /organism=“Artificial Sequence” /note=“Description of Artificial Sequence Synthetic oligonucleotide” /mol_type=“unassigned DNA”
ggggttttggggttttggggttttgggg

SEQ ID NO: 7 - DNA, 24 bp, Artificial Sequence
  source 1..24: /organism=“Artificial Sequence” /note=“Description of Artificial Sequence Synthetic oligonucleotide” /mol_type=“unassigned DNA”
ttggggttggggttggggttgggg

SEQ ID NO: 8 - DNA, 20 bp, Artificial Sequence
  source 1..20: /organism=“Artificial Sequence” /note=“Description of Artificial Sequence Synthetic oligonucleotide” /mol_type=“unassigned DNA”
ggttttggttttggttttgg

SEQ ID NO: 9 - DNA, 23 bp, Artificial Sequence
  source 1..23: /organism=“Artificial Sequence” /note=“Description of Artificial Sequence Synthetic oligonucleotide” /mol_type=“unassigned DNA”
ggggttggggtgtggggttgggg

SEQ ID NO: 10 - DNA, 29 bp, Artificial Sequence
  source 1..29: /organism=“Artificial Sequence” /note=“Description of Artificial Sequence Synthetic oligonucleotide” /mol_type=“unassigned DNA”
tttggtggtggtggttgtggtggtggtgg

SEQ ID NO: 11 - DNA, 34 bp, Artificial Sequence
  source 1..34: /organism=“Artificial Sequence” /note=“Description of Artificial Sequence Synthetic oligonucleotide” /mol_type=“unassigned DNA”
ggttggttccatgacgttcctgatctgtggttgg

SEQ ID NO: 12 - DNA, 47 bp, Artificial Sequence
  source 1..47: /organism=“Artificial Sequence” /note=“Description of Artificial Sequence Synthetic oligonucleotide” /mol_type=“unassigned DNA”
ggggttttggtccatgacgttcctgatctggttttggggttttgggg

SEQ ID NO: 13 - DNA, 43 bp, Artificial Sequence
  source 1..43: /organism=“Artificial Sequence” /note=“Description of Artificial Sequence Synthetic oligonucleotide” /mol_type=“unassigned DNA”
ttggggttggtccatgacgttcctgatctggttggggttgggg

SEQ ID NO: 14 - DNA, 39 bp, Artificial Sequence
  source 1..39: /organism=“Artificial Sequence” /note=“Description of Artificial Sequence Synthetic oligonucleotide” /mol_type=“unassigned DNA”
ggttttgtccatgacgttcctgatctgttttggttttgg

SEQ ID NO: 15 - DNA, 42 bp, Artificial Sequence
  source 1..42: /organism=“Artificial Sequence” /note=“Description of Artificial Sequence Synthetic oligonucleotide” /mol_type=“unassigned DNA”
ggggttggggtgtggtccatgacgttcctgatctggttgggg

SEQ ID NO: 16 - DNA, 48 bp, Artificial Sequence
  source 1..48: /organism=“Artificial Sequence” /note=“Description of Artificial Sequence Synthetic oligonucleotide” /mol_type=“unassigned DNA”
tttggtggtgtccatgacgttcctgatctgtggttgtggtggtggtgg

SEQ ID NO: 17 - DNA, 30 bp, Artificial Sequence
  source 1..30: /organism=“Artificial Sequence” /note=“Description of Artificial Sequence Synthetic oligonucleotide” /mol_type=“unassigned DNA”
ggttggtgctagacgttagcgtgtggttgg

SEQ ID NO: 18 - DNA, 43 bp, Artificial Sequence
  source 1..43: /organism=“Artificial Sequence” /note=“Description of Artificial Sequence Synthetic oligonucleotide” /mol_type=“unassigned DNA”
ggggttttgggctagacgttagcgtggttttggggttttgggg

SEQ ID NO: 19 - DNA, 39 bp, Artificial Sequence
  source 1..39: /organism=“Artificial Sequence” /note=“Description of Artificial Sequence Synthetic oligonucleotide” /mol_type=“unassigned DNA”
ttggggttgggctagacgttagcgtggttggggttgggg

SEQ ID NO: 20 - DNA, 35 bp, Artificial Sequence
  source 1..35: /organism=“Artificial Sequence” /note=“Description of Artificial Sequence Synthetic oligonucleotide” /mol_type=“unassigned DNA”
ggttttggctagacgttagcgtgttttggttttgg

SEQ ID NO: 21 - DNA, 38 bp, Artificial Sequence
  source 1..38: /organism=“Artificial Sequence” /note=“Description of Artificial Sequence Synthetic oligonucleotide” /mol_type=“unassigned DNA”
ggggttggggtgtgggctagacgttagcgtggttgggg

SEQ ID NO: 22 - DNA, 44 bp, Artificial Sequence
  source 1..44: /organism=“Artificial Sequence” /note=“Description of Artificial Sequence Synthetic oligonucleotide” /mol_type=“unassigned DNA”
tttggtggtggctagacgttagcgtgtggttgtggtggtggtgg

SEQ ID NO: 23 - DNA, 35 bp, Artificial Sequence
  source 1..35: /organism=“Artificial Sequence” /note=“Description of Artificial Sequence Synthetic oligonucleotide” /mol_type=“unassigned DNA”
ggttggtatcgactctcgagcgttctcgtggttgg

SEQ ID NO: 24 - DNA, 48 bp, Artificial Sequence
  source 1..48: /organism=“Artificial Sequence” /note=“Description of Artificial Sequence Synthetic oligonucleotide” /mol_type=“unassigned DNA”
ggggttttggatcgactctcgagcgttctcggttttggggttttgggg

SEQ ID NO: 25 - DNA, 44 bp, Artificial Sequence
  source 1..44: /organism=“Artificial Sequence” /note=“Description of Artificial Sequence Synthetic oligonucleotide” /mol_type=“unassigned DNA”
ttggggttggatcgactctcgagcgttctcggttggggttgggg

SEQ ID NO: 26 - DNA, 40 bp, Artificial Sequence
  source 1..40: /organism=“Artificial Sequence” /note=“Description of Artificial Sequence Synthetic oligonucleotide” /mol_type=“unassigned DNA”
ggttttgatcgactctcgagcgttctcgttttggttttgg

SEQ ID NO: 27 - DNA, 43 bp, Artificial Sequence
  source 1..43: /organism=“Artificial Sequence” /note=“Description of Artificial Sequence Synthetic oligonucleotide” /mol_type=“unassigned DNA”
ggggttggggtgtggatcgactctcgagcgttctcggttgggg

SEQ ID NO: 28 - DNA, 49 bp, Artificial Sequence
  source 1..49: /organism=“Artificial Sequence” /note=“Description of Artificial Sequence Synthetic oligonucleotide” /mol_type=“unassigned DNA”
tttggtggtgatcgactctcgagcgttctcgtggttgtggtggtggtgg